(19) United States
(12) Patent Application Publication
(10) Pub. No.: US 2022/0315527 A1
(43) Pub. Date: Oct. 6, 2022

(54) CALIXARENE COMPOUND, CURABLE COMPOSITION AND CURED PRODUCT

(71) Applicant: DIC Corporation, Tokyo (JP)

(72) Inventors: Shinya Yamamoto, Sakura-shi (JP); Masanori Miyamoto, Sakura-shi (JP); Hidetomo Kai, Sakura-shi (JP); Tomoyuki Imada, Ichihara-shi (JP); Yutaka Kadomoto, Kita-adachi-gun (JP)

(73) Assignee: DIC Corporation, Tokyo (JP)

(21) Appl. No.: 17/054,607

(22) PCT Filed: May 15, 2019
(86) PCT No.: PCT/JP2019/019365

§ 371 Date: Nov. 11, 2020

(30) Foreign Application Priority Data
May 15, 2018 (JP) 2018-093763
May 15, 2018 (JP) 2018-093764
May 15, 2018 (JP) 2018-093765
May 15, 2018 (JP) 2018-093766
May 15, 2018 (JP) 2018-093767

(51) Int. Cl. C07C 255/13; C07C 69/732; C07C 255/23; C07C 69/593; C07C 69/716; C07C 69/36; C08F 20/34; C08F 20/30

(52) U.S. Cl. CPC C07C 255/13; C07C 69/732; C07C 255/23; C08F 20/30; C07C 69/716; C07C 69/36; C08F 20/34; C07C 69/593

(57) ABSTRACT

Provided is a calixarene compound represented by structural formula (1). In structural formula (1), R¹ and R² each independently represent a structural moiety (A) having a functional group (I), a structural moiety (B) having a functional group (II) having a carbon-carbon unsaturated bond (excluding maleate groups), a structural moiety (C) having both the functional group (I) and the functional group (II), a monovalent organic group (D) that has 1 to 20 carbon atoms and is other than the structural moieties (A), (B) and (C), or a hydrogen atom (E). At least one of a plurality of R²s is the structural moiety (A), the structural moiety (B), the structural moiety (C), or the organic group (D).

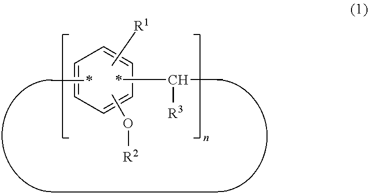

### TECHNICAL FIELD

The present invention relates to a calixarene compound having a novel structure, to a curable composition containing the calixarene compound, and to a cured product of the curable composition.

### BACKGROUND ART

Calixarenes are cyclic oligomers (macrocyclic phenolic resin derivatives) generated by condensation of phenols and formaldehyde. Calixarenes and derivatives thereof have a specific inverted calix-like structure formed of benzene rings and are therefore known to have inclusion properties as do crown ethers and cyclodextrins. Therefore, researches using calixarenes and their derivatives as third host molecules (such as researches aimed at recovery of heavy metals from seawater) have been actively conducted in recent years. However, with a few exceptions, they have not yet been in practical use.

In some products such as semiconductor devices including ICs, LSI devices, etc. and display devices including slim displays etc., photosensitive resin coating films are formed on components included in the products or between components, and the coating films are used as members that remain present in the completed products (members conceptually referred to as permanent films). Specific examples of the permanent films for semiconductor devices include solder resists, packaging materials, underfill materials, package bonding layers for circuit elements etc., and bonding layers for bonding integrated circuit elements to circuit boards. Specific examples of the permanent films for slim displays typified by LCDs and OLEDs include thin-film transistor protective films, liquid crystal color filter protective films, black matrixes, spacers, bank materials, partition forming materials, and cover materials. Negative resists using (meth)acrylate-based polymers are widely used as resists for the permanent films. Specifically, in one commonly used method, silica, a pigment, etc. is dispersed in a photocurable polymer solution. In recent display devices, the distance between their display unit and their light source is decreasing due to their finer design and their reduced thickness, and therefore one task is to achieve a reduction in line width and heat resistance simultaneously. However, with the above method, it is difficult to achieve them simultaneously. Moreover, in general, a polar group is introduced into the resist resin in order to adhere the resist resin to a silicon substrate. However, this causes a problem in that the resist resin can swell with water.

Therefore, in applications that require finer design and higher functionality, there is a strong need for a novel material with which adhesion to a substrate, solubility in a general-purpose solvent, heat resistance of its cured product, and the thermal stability thereof, etc. can be achieved in a balanced manner.

For example, PTL 1 and PTL 2 each disclose a technique in which reactive functional groups are introduced into a calixarene to thereby prepare a curable resin composition. However, these curable resin compositions do not have performance sufficient for the above applications that require finer design and higher functionality.

### CITATION LIST

### Patent Literature

PTL 1: Japanese Unexamined Patent Application Publication No. 9-263560

PTL 2: Japanese Unexamined Patent Application Publication No. 11-72916

### SUMMARY OF INVENTION

### Technical Problem

One object to be achieved by the present invention is to provide a calixarene compound having a novel structure that can provide a cured product excellent not only in properties such as heat resistance and hardness but also in properties such as adhesion to a substrate. Another object to be achieved by the present invention is to provide a curable composition containing the calixarene compound and a cured product thereof.

### Solution to Problem

The present inventors have conducted extensive studies to achieve the above objects and found that a cured product excellent not only in properties such as heat resistance and hardness but also in properties such as adhesion to a substrate can be obtained by a calixarene compound having a specific functional group and a carbon-carbon unsaturated bond. Thus, the present invention has been completed.

Accordingly, the present invention provides a calixarene compound represented by structural formula (1) below, a curable composition containing the calixarene compound, and a cured product of the curable composition.

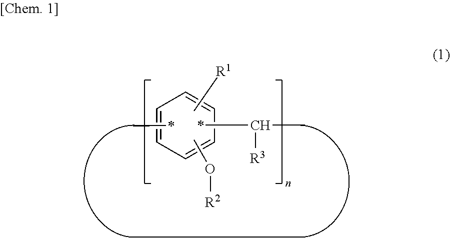

In formula (1),

R¹ and R² each independently represent a structural moiety (A) having a functional group (I) selected from the group consisting of a cyano group, maleate groups, an acetylacetonate group, oxalate groups, and malonate groups, a structural moiety (B) having a functional group (II) having a carbon-carbon unsaturated bond (excluding maleate groups), a structural moiety (C) having both the functional group (I) and the functional group (II), a monovalent organic group (D) that has 1 to 20 carbon atoms and is other than the structural moieties (A), (B) and (C), or a hydrogen atom (E);

R³ represents a hydrogen atom, an aliphatic hydrocarbon group optionally having a substituent, or an aryl group optionally having a substituent;

n is an integer of 2 to 10; and

*s indicate the points of attachment to the aromatic ring.

A plurality of R¹s may be the same or different; a plurality of R²s may be the same or different; and a plurality of R³s may be the same or different,

provided that at least one of the plurality of R²s is the structural moiety (A), the structural moiety (B), the structural moiety (C), or the organic group (D).

When the functional group (I) is a cyano group, an acetylacetonate group, an oxalate group, or a malonate group, at least one of the plurality of R¹s and the plurality of R²s is the structural moiety (C), or at least one of the plurality of R¹s and the plurality of R²s is the structural moiety (A) and at least another one of the plurality of R¹s and the plurality of R²s is the structural moiety (B).

When the functional group (I) is a maleate group, at least one of the plurality of R¹s and the plurality of R²s is the structural moiety (A) or the structural moiety (C).

### Advantageous Effects of Invention

The present invention can provide a calixarene compound having a novel structure that allows good solubility in a general purpose solvent and can provide a cured product excellent not only in properties such as heat resistance and hardness but also in properties such as adhesion to a substrate. Moreover, the present invention can provide a curable composition containing the calixarene compound and a cured product thereof. The calixarene compound of the present invention can be preferably used for various applications such as paints, printing inks, adhesives, resist materials, and interlayer dielectrics.

### BRIEF DESCRIPTION OF DRAWINGS

FIG. 1 is an FD-MS chart of calixarene compound 17-6 obtained in Example 21 in Example group <I>.

FIG. 2 in a ¹H-NMR chart of calixarene compound 17-6 obtained in Example 21 in Example group <I>.

FIG. 3 is a ¹³C-NMR chart of calixarene compound 17-6 obtained in Example 21 in Example group <I>.

FIG. 4 is a ¹H-NMR chart of calixarene compound 19-6 obtained in Example 31 in Example group <I>.

FIG. 5 is a ¹H-NMR chart of calixarene compound 32-18 obtained in Example 44 in Example group <I>.

FIG. 6 is an FD-MS chart of calixarene compound 33-7 obtained in Example 13 in Example group <II>.

FIG. 7 is a ¹H-NMR chart of calixarene compound 33-7 obtained in Example 13 in Example group <II>.

FIG. 8 is a ¹³C-NMR chart of calixarene compound 33-7 obtained in Example 13 in Example group <II>.

FIG. 9 is an FD-MS chart of calixarene compound 17-6 obtained in Example 9 in Example group <III>.

FIG. 10 is a ¹H-NMR chart of calixarene compound 17-6 obtained in Example 9 in Example group <III>.

FIG. 11 is a ¹³C-NMR chart of calixarene compound 17-6 obtained in Example 9 in Example group <III>.

FIG. 12 is a ¹H-NMR chart of calixarene compound 18-18 obtained in Example 12 in Example group <III>.

FIG. 13 is a ¹³C-NMR chart of calixarene compound 18-18 obtained in Example 12 in Example group <III>.

FIG. 14 is an FD-MS chart of calixarene compound 33-7 obtained in Example 13 in Example group <IV>.

FIG. 15 is a ¹H-NMR chart of calixarene compound 33-7 obtained in Example 13 in Example group <IV>.

FIG. 16 is a ¹³C-NMR chart of calixarene compound 33-7 obtained in Example 13 in Example group <IV>.

FIG. 17 is a ¹H-NMR chart of calixarene compound 35-7 obtained in Example 13 in Example group <IV>.

FIG. 18 is an FD-MS chart of calixarene compound 33-6 obtained in Example 13 in Example group <V>.

FIG. 19 is a ¹H-NMR chart of calixarene compound 33-6 obtained in Example 13 in Example group <V>.

FIG. 20 is a ¹³C-NMR chart of calixarene compound 33-6 obtained in Example 13 in Example group <V>.

FIG. 21 is a ¹H-NMR chart of calixarene compound 41-6 obtained in Example 19 in Example group <V>.

FIG. 22 is a ¹H-NMR chart of calixarene compound 42-6 in Example 19 in Example group <V>.

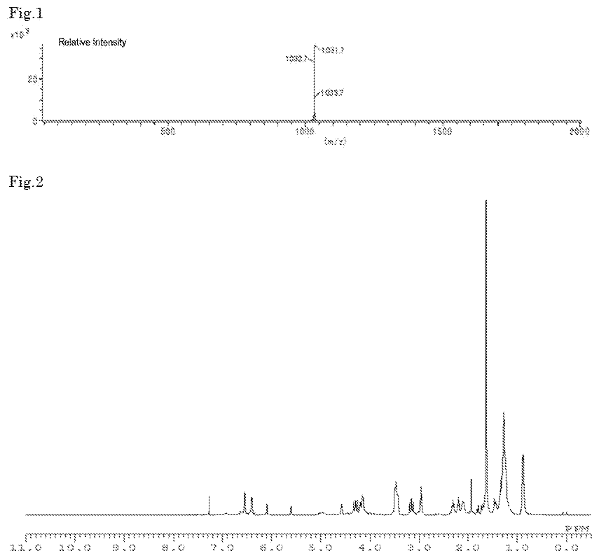

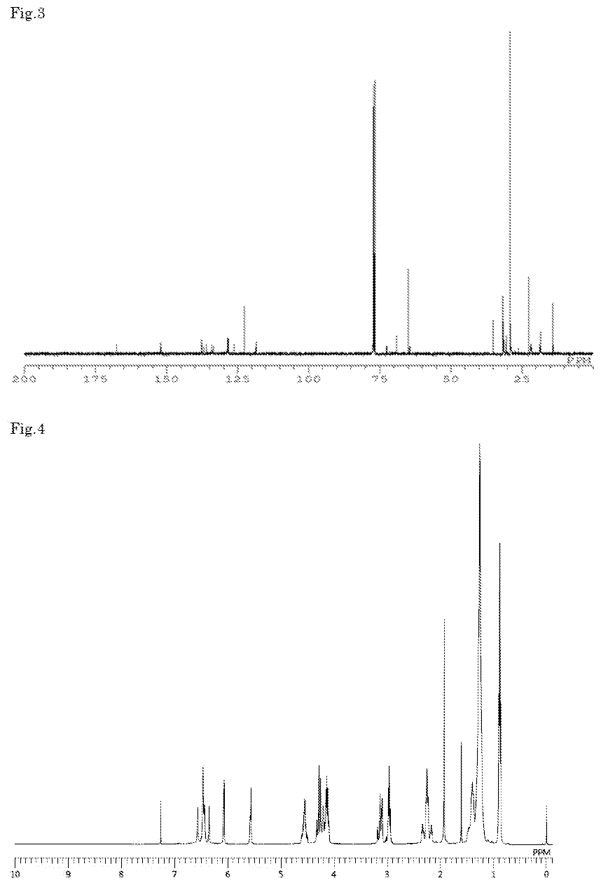

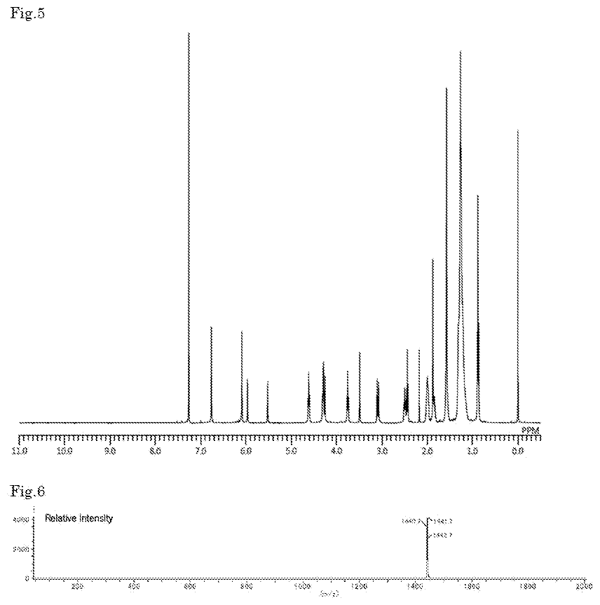

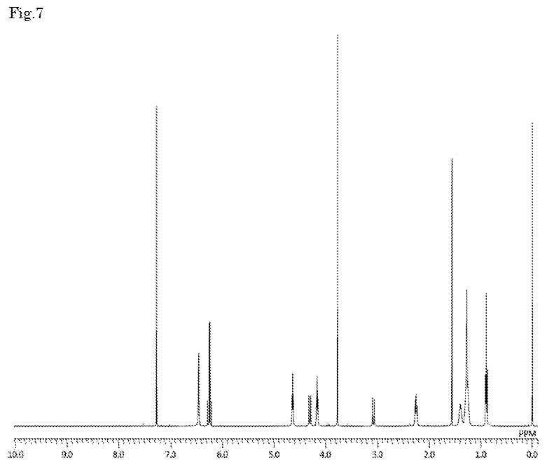

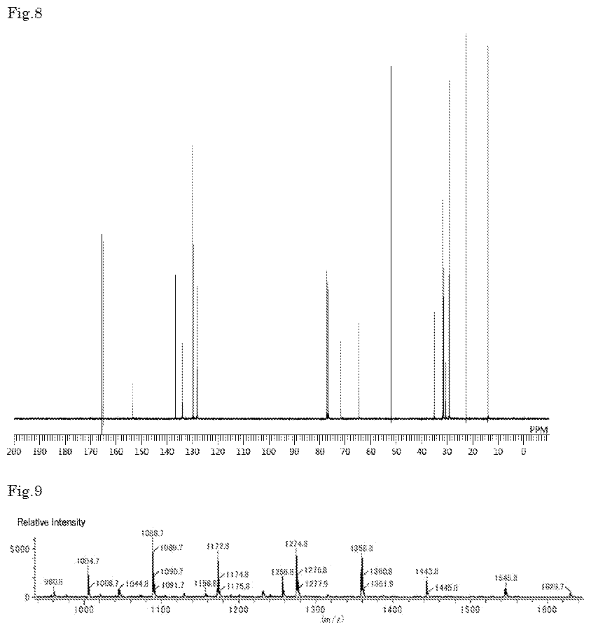

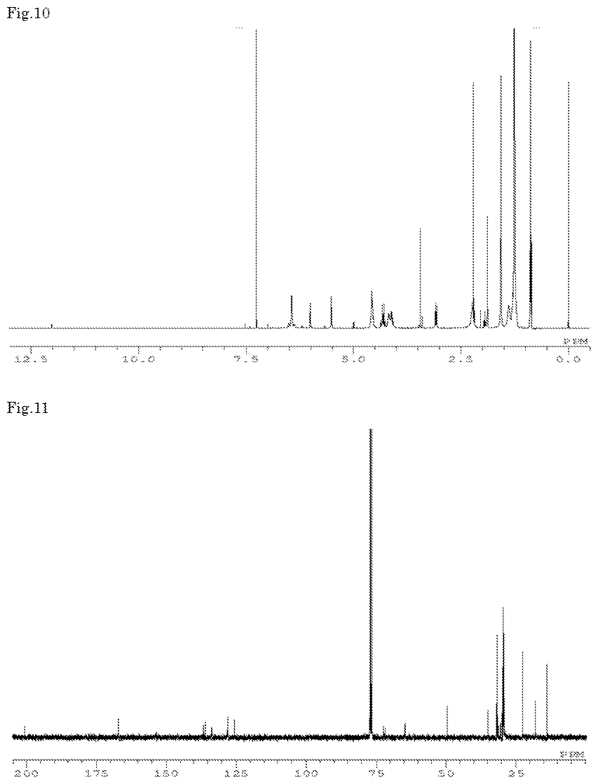

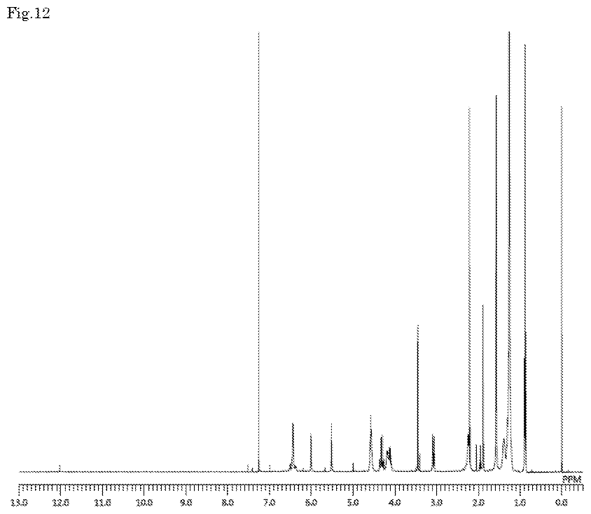

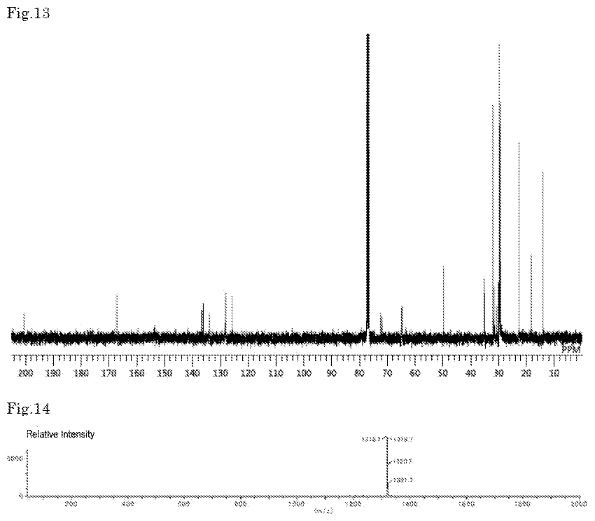

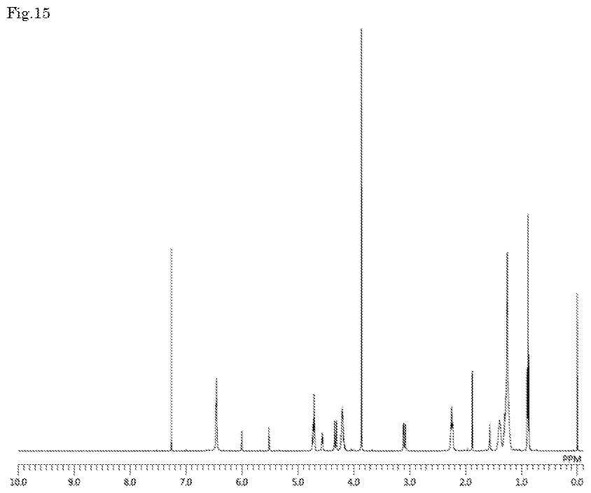

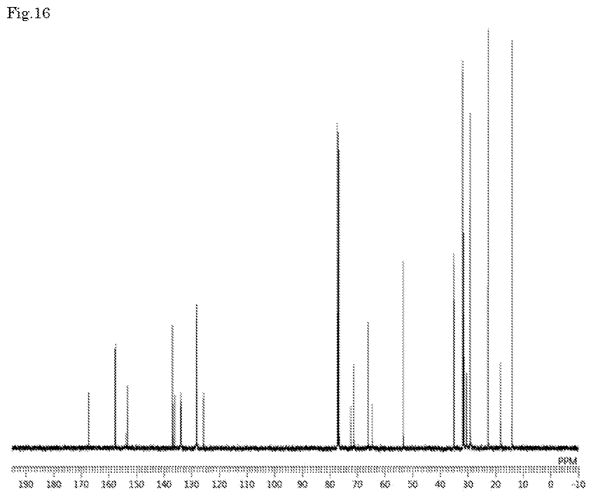

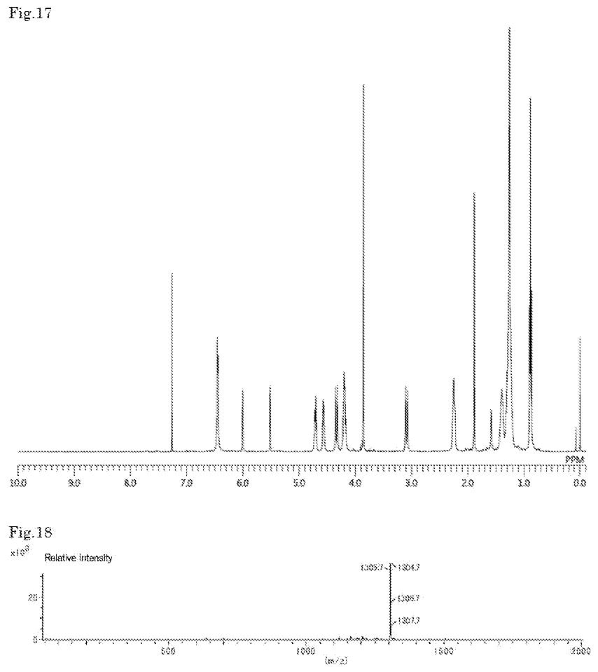

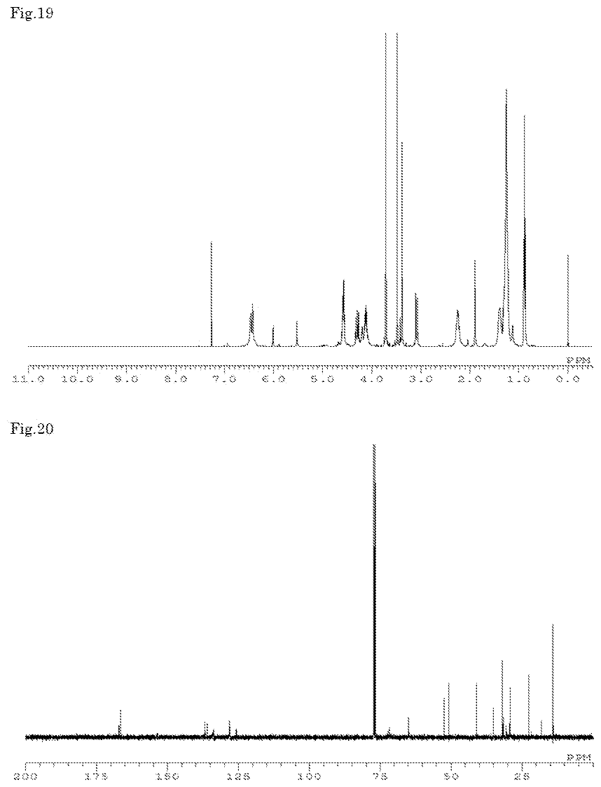

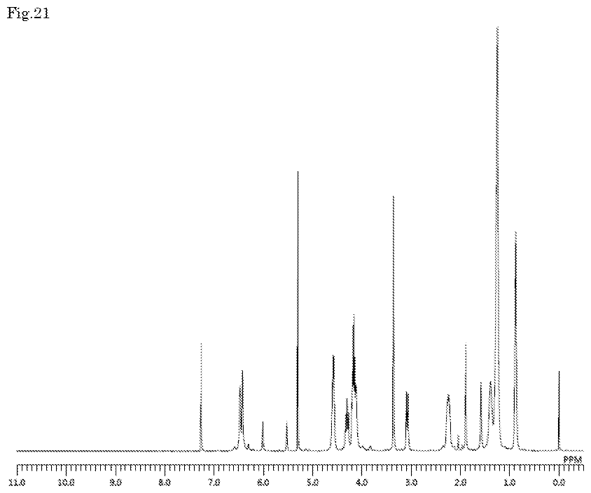

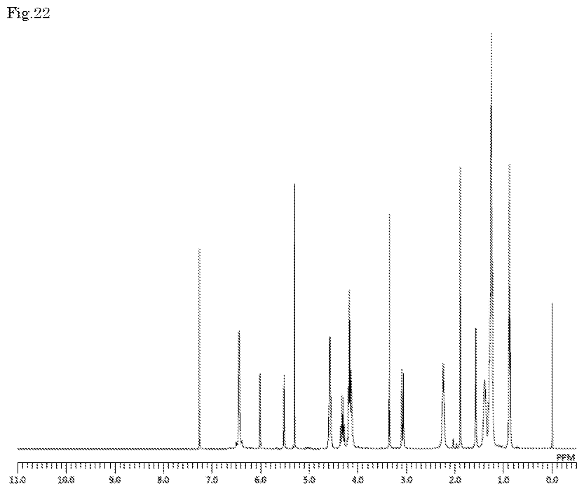

### DESCRIPTION OF EMBODIMENTS

Preferred embodiments of the present invention will be described in detail.

A calixarene compound in an embodiment is a compound represented by structural formula (1) below.

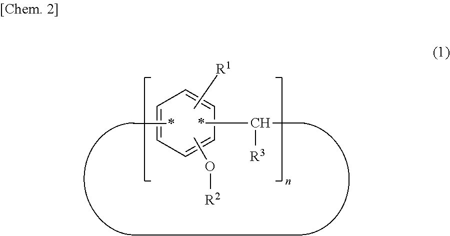

In formula (1),

R¹ and R² each independently represent a structural moiety (A) having a functional group (I) selected from the group consisting of a cyano group, maleate groups, an acetylacetonate group, oxalate groups, and malonate groups, a structural moiety (B) having a functional group (II) having a carbon-carbon unsaturated bond (excluding maleate groups), a structural moiety (C) having both the functional group (I) and the functional group (II), a monovalent organic group (D) that has 1 to 20 carbon atoms and is other than the structural moieties (A), (B) and (C), or a hydrogen atom (E);

R³ represents a hydrogen atom, an aliphatic hydrocarbon group optionally having a substituent, or an aryl group optionally having a substituent;

n is an integer of 2 to 10; and

*s indicate the points of attachment to the aromatic ring.

A plurality of R²s may be the same or different; a plurality of R²s may be the same or different; and a plurality of R³s may be the same or different.

However, at least one of the plurality of R²s is the structural moiety (A), the structural moiety (B), the structural moiety (C), or the organic group (D). Specifically, a compound in which all R²s are hydrogen atoms (E) is excluded from the compound of structural formula (1).

When the functional group (I) is a cyano group, an acetylacetonate group, an oxalate group, or a malonate group, at least one of the plurality of R²s and the plurality of R²s is the structural moiety (C), or at least one of the plurality of R¹s and the plurality of R²s is the structural moiety (A) and at least another one of the plurality of R²s and the plurality of R²s is the structural moiety (B). When the functional group (I) is a maleate group, at least one of the plurality of R¹s and the plurality of R²s is the structural moiety (A) or the structural moiety (C). Specifically, the calixarene compound in the present embodiment has at least one functional group (I) and at least one carbon-carbon unsaturated bond.

In structural formula (1), n is an integer of 2 to 10. In particular, n is preferably 4, 6, or 8 and particularly preferably 4 because a stable structure is obtained and the structural features of the calixarene compound become remarkable.

In structural formula (1), R¹ and R² each represent the structural moiety (A), the structural moiety (B), the structural moiety (C), the organic group (D), or a hydrogen atom (E). The plurality of R¹s present in the molecule may have the same structure or may have different structures, and the plurality of R²s present in the molecule may have the same structure or may have different structures. The structural moieties (A) to (D) will be described in detail.

<Structural Moiety (A)>

(i) In the Case where the Functional Group (I) is a Cyano Group

In the structural moiety (A) having a cyano group, no particular limitation is imposed on the specific structure of the structural moiety (A) excluding the cyano group so long as the structural moiety (A) has one or a plurality of cyano groups. Example of the structural moiety (A) include a (poly)cyanoalkyl group (A-1) and a group represented by structural formula (A-2) below.

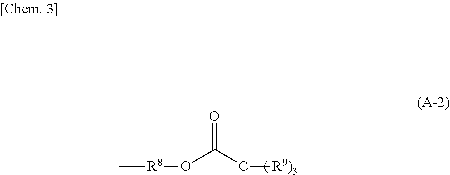

In formula (A-2), R⁸ is an aliphatic hydrocarbon group or a direct bond. R⁹s are each independently a hydrogen atom, a hydroxy group, an alkyl group, or a (poly)cyanoalkyl group, and at least one of R⁹s is a (poly)cyanoalkyl group.

The (poly)cyanoalkyl group (A-1) is an alkyl group substituted with a plurality of cyano groups. In the (poly)cyanoalkyl group (A-1), the alkyl group serving as the main skeleton may be linear or branched, and no particular limitation is imposed on the number of carbon atoms in the alkyl group. In particular, the number of carbon atoms in the alkyl group is preferably in the range of 1 to 20 and more preferably in the range of 1 to 12 because better properties such as adhesion to a substrate are obtained while the heat resistance and robustness of the calixarene compound are maintained. The number of cyano groups is preferably in the range of 1 to 3.

As for the group represented by structural formula (A-2), R⁸ in structural formula (A-2) is an aliphatic hydrocarbon group or a direct bond. The aliphatic hydrocarbon group may be linear or branched. The aliphatic hydrocarbon group may have a cyclic ring structure as a partial structure. In particular, R⁸ is preferably an alkanediyl group and more preferably a liner alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6.

In structural formula (A-2), R⁹s are each independently a hydrogen atom, a hydroxy group, an alkyl group, or a (poly)cyanoalkyl group, and at least one of R⁹s is a (poly)cyanoalkyl group. The alkyl group may be linear or branched, and no particular limitation is imposed on the number of carbon atoms in the alkyl group. In particular, the number of carbon atoms in the alkyl group is preferably in the range of 1 to 12 and more preferably in the range of 1 to 6 because better properties such as adhesion to a substrate are obtained while the heat resistance and robustness of the calixarene compound are maintained. Examples of the (poly)cyanoalkyl group include the same groups as those for the (poly)cyanoalkyl group (A-1). The number of carbon atoms in the alkyl group serving as the main skeleton of the (poly)cyanoalkyl group is preferably in the range of 1 to 12 and more preferably in the range of 1 to 6 because better properties such as adhesion to a substrate are obtained while the heat resistance and robustness of the calixarene compound are maintained. The number of cyano groups is preferably 1 to 3.

(ii) In the Case where the Functional Group (I) is a Maleate Group

In the structural moiety (A) having a maleate group, no particular limitation is imposed on the specific structure of the structural moiety (A) excluding the maleate group so long as the structural moiety (A) has one or a plurality of maleate groups. One example of the structural moiety (A) is a group represented by structural formula (A-1) below.

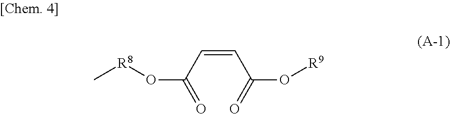

In formula (A-1), R⁸ is an aliphatic hydrocarbon group or a direct bond, and R⁹ is an aliphatic hydrocarbon group.

As for the group represented by structural formula (A-1), R⁸ in structural formula (A-1) is an aliphatic hydrocarbon group or a direct bond. R⁹ is an aliphatic hydrocarbon group. These aliphatic hydrocarbon groups may be linear or branched. The aliphatic hydrocarbon groups may have a cyclic ring structure as a partial structure. R⁸ in structural formula (A-1) is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. R⁹ in structural formula (A-1) is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

(iii) In the Case where the Functional Group (I) is an Acetylacetonate Group

In the structural moiety (A) having an acetylacetonate group, no particular limitation is imposed on the specific structure of the structural moiety (A) excluding the acetylacetonate group so long as the structural moiety (A) has one or a plurality of acetylacetonate groups. One example of the structural moiety (A) is a group represented by structural formula (A-1) below.

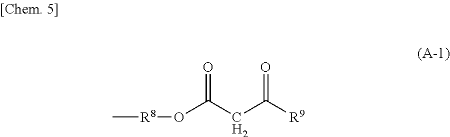

In formula (A-1), R⁸ is an aliphatic hydrocarbon group or a direct bond, and R⁹ is an aliphatic hydrocarbon group.

As for the group represented by structural formula (A-1), R⁸ in structural formula (A-1) is an aliphatic hydrocarbon group or a direct bond. R⁹ is an aliphatic hydrocarbon group. These aliphatic hydrocarbon groups may be linear or branched. The aliphatic hydrocarbon groups may have a cyclic ring structure as a partial structure. R⁸ in structural formula (A-1) is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. R⁹ in structural formula (A-1) is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

(iv) In the Case where the Functional Group (I) is an Oxalate Group

In the structural moiety (A) having an oxalate group, no particular limitation is imposed on the specific structure of the structural moiety (A) excluding the oxalate group so long as the structural moiety (A) has one or a plurality of oxalate groups. One example of the structural moiety (A) is a group represented by structural formula (A-1) below.

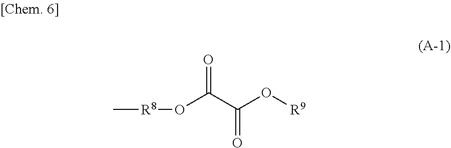

In formula (A-1), R⁸ is an aliphatic hydrocarbon group or a direct bond, and R⁹ is an aliphatic hydrocarbon group.

As for the group represented by structural formula (A-1), R⁸ in structural formula (A-1) is an aliphatic hydrocarbon group or a direct bond. R⁹ is an aliphatic hydrocarbon group. These aliphatic hydrocarbon groups may be linear or branched. The aliphatic hydrocarbon groups may have a cyclic ring structure as a partial structure. R⁸ in structural formula (A-1) is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. R⁹ in structural formula (A-1) is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

(v) In the Case where the Functional Group (I) is a Malonate

In the structural moiety (A) having a malonate group, no particular limitation is imposed on the specific structure of the structural moiety (A) excluding the malonate group so long as the structural moiety (A) has one or a plurality of malonate groups. One example of the structural moiety (A) is a group represented by structural formula (A-1) below.

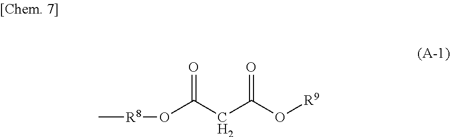

In formula (A-1), R⁸ is an aliphatic hydrocarbon group or a direct bond, and R⁹ is an aliphatic hydrocarbon group.

As for the group represented by structural formula (A-1), R⁸ in structural formula (A-1) is an aliphatic hydrocarbon group or a direct bond. R⁹ is an aliphatic hydrocarbon group. These aliphatic hydrocarbon groups may be linear or branched. The aliphatic hydrocarbon groups may have a cyclic ring structure as a partial structure. R⁸ in structural formula (A-1) is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. R⁹ in structural formula (A-1) is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

<Structural Moiety (B)>

The structural moiety (B) has the functional group (II) having a carbon-carbon unsaturated bond. No particular limitation is imposed on the specific structure of the structural moiety (B) excluding the functional group (II) so long as the structural moiety (B) has one or a plurality of functional groups (II). The functional group (II) has a carbon-carbon unsaturated bond. No particular limitation is imposed on the specific structure of the functional group (II) so long as it has one or a plurality of carbon-carbon unsaturated bonds, but maleate groups are excluded. The carbon-carbon unsaturated bond is specifically an ethylenic double bond or an acetylenic triple bond. In the present description, the carbon-carbon unsaturated bond does not include unsaturated bonds in aromatic rings. Preferably, the structural moiety (B) and the functional group (II) have an ethylenic double bond.

Examples of the structural moiety (B) include a vinyl group, a propargyl group, a (meth)acryloyl group, a (meth)acryloylamino group, a group represented by structural formula (B-1) below, and a group represented by structural formula (B-2) below.

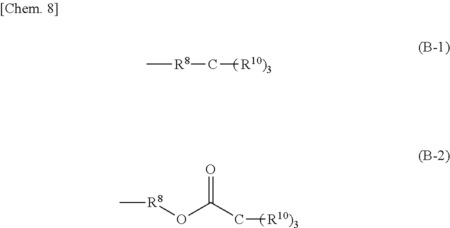

In formulas (B-1) and (B-2), R⁸s are each independently an aliphatic hydrocarbon group or a direct bond. R¹⁰s are each independently a hydrogen atom, an alkyl group, a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group. At least one of the three R¹⁰s in each of the formulas is a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group.

R⁸ in each of structural formulas (B-1) and (B-2) is an aliphatic hydrocarbon group or a direct bond. The aliphatic hydrocarbon group may be linear or branched and may have an unsaturated bond in its structure. The aliphatic hydrocarbon group may have a cyclic ring structure as a partial structure. In particular, R⁸ is preferably a direct bond or an alkanediyl group because better properties such as adhesion to a substrate are obtained while the heat resistance and robustness of the calixarene compound are maintained. The number of carbon atoms in the alkanediyl group is preferably in the range of 1 to 12 and more preferably in the range of 1 to 6.

R¹⁰s in structural formulas (B-1) and (B-2) are each independently a hydrogen atom, an alkyl group, a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group. At least one of the three R¹⁰s in structural formula (B-1) is a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group. At least one of the three R¹⁰s in structural formula (B-2) is a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group.

As for R¹⁰s in structural formulas (B-1) and (B-2), the alkyl group may be linear or branched, and no particular limitation is imposed on the number of carbon atoms in the alkyl group. In particular, the number of carbon atoms in the alkyl group is preferably in the range of 1 to 12 and more preferably in the range of 1 to 6 because better properties such as adhesion to a substrate are obtained while the heat resistance and robustness of the calixarene compound are maintained.

As for R¹⁰s in structural formulas (B-1) and (B-2), the alkyl moieties in the vinyloxyalkyl group, the allyloxyalkyl group, the propargyloxyalkyl group, the (meth)acryloyloxyalkyl group, and the (meth)acryloylaminoalkyl group may be linear or branched, and no particular limitation is imposed on the number of carbon atoms in each of the alkyl moieties. In particular, the number of carbon atoms in each alkyl moiety is preferably in the range of 1 to 12 and more preferably in the range of 1 to 6 because better properties such as adhesion to a substrate are obtained while the heat resistance and robustness of the calixarene compound are maintained.

<Structural Moiety (C)>

(i) In the Case where the Functional Group (I) is a Cyano Group

In the structural moiety (C) having both a cyano group and a carbon-carbon unsaturated bond (the functional group (II)), no particular limitation is imposed on the specific structure of the structural moiety (C) excluding the cyano group and the carbon-carbon unsaturated bond so long as the structural moiety (C) has at least one cyano group and at least one carbon-carbon unsaturated bond. Examples of the specific structure include groups represented by structural formulas (C-1) to (C-3) below.

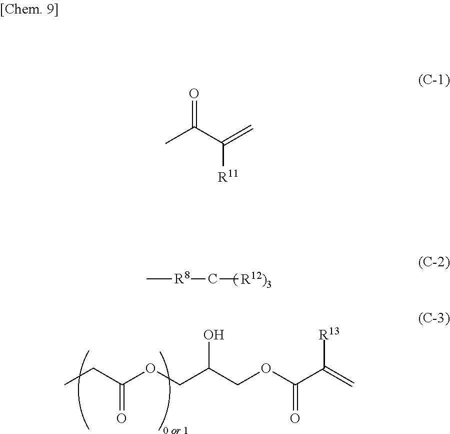

In formulas (C-1) to (C-3), R¹¹ is a (poly)cyanoalkyl group. R⁸ is an aliphatic hydrocarbon group or a direct bond. R¹²s are each independently a hydrogen atom, an alkyl group, a hydroxy group, a (poly)cyanoalkyl group, a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, a (meth)acryloylaminoalkyl group, or a group represented by structural formula (C-2-1) below:

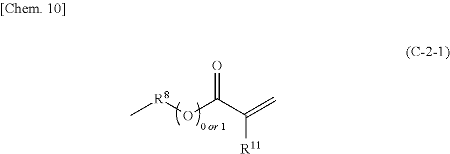

(wherein R⁸ and R¹¹ are the same as described above). R¹³ is a (poly) cyanoalkyl group. At least one of the three R¹²s in formula (C-2) is a group represented by structural formula (C-2-1). Alternatively, at least one of the three R¹²s is a (poly)cyanoalkyl group, and at least another one is a vinyl group, a vinyloxy group, an allyl group, an allyloxy group, a propargyl group, a propargyloxy group, a (meth)acryloyl group, a (meth)acryloyloxy group, a (meth)acryloyloxyalkylene group, a (meth)acryloylamino group, or a (meth)acryloylaminoalkylene group.

As for R¹¹ in each of structural formulas (C-1) and (C-2-1), examples of the (poly)cyanoalkyl group include the same groups as those for the (poly)cyanoalkyl group (A-1). In particular, the number of carbon atoms in the alkyl group serving as the main skeleton of the (poly)cyanoalkyl group is preferably in the range of 1 to 12 and more preferably in the range of 1 to 6 because better properties such as adhesion to a substrate are obtained while the heat resistance and robustness of the calixarene compound are maintained. The number of cyano groups is preferably in the range of 1 to 3.

R⁸ in each of structural formulas (C-2) and (C-2-1) is an aliphatic hydrocarbon group or a direct bond. The aliphatic hydrocarbon group may be linear or branched and may have an unsaturated bond in its structure. The aliphatic hydrocarbon group may have a cyclic ring structure as a partial structure. In particular, R⁸ is preferably an alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6.

R¹²s in structural formula (C-2) are each independently a hydrogen atom, an alkyl group, a (poly)cyanoalkyl group, a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, a (meth)acryloylaminoalkyl group, or a group represented by structural formula (C-2-1) above. The alkyl group in each R¹² may be linear or branched, and no particular limitation is imposed on the number of carbon atoms in the alkyl group. In particular, the number of carbon atoms in the alkyl group in each R¹² is preferably in the range of 1 to 12 and more preferably in the range of 1 to 6 because good properties such as adhesion to a substrate are obtained while the heat resistance and robustness of the calixarene compound are maintained.

As for R¹³ in structural formula (C-3), examples of the (poly)cyanoalkyl group include the same groups as those for the (poly)cyanoalkyl group (A-1). In particular, the number of carbon atoms in the alkyl group serving as the main skeleton of the (poly)cyanoalkyl group is preferably in the range of 1 to 12 and more preferably in the range of 1 to 6 because better properties such as adhesion to a substrate are obtained while the heat resistance and robustness of the calixarene compound are maintained. The number of cyano groups is preferably in the range of 1 to 3.

(ii) In the Case where the Functional Group (I) is a Maleate Group

In the structural moiety (C) having both a maleate group and a carbon-carbon unsaturated bond other than maleate groups (the functional group (II)), no particular limitation is imposed on the specific structure of the structural moiety (C) excluding the maleate group and the carbon-carbon unsaturated bond so long as the structural moiety (C) has at least one maleate group and at least one carbon-carbon unsaturated bond other than maleate groups. One example of the specific structure is a group represented by structural formula (C-1) below.

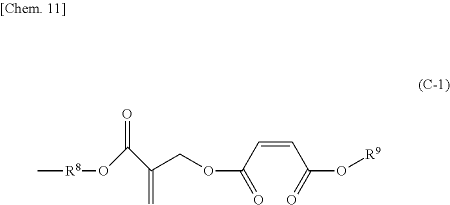

In formula (C-1), R⁸ is an aliphatic hydrocarbon group or a direct bond, and R⁹ is an aliphatic hydrocarbon group.

In structural formula (C-1), R⁸ is an aliphatic hydrocarbon group or a direct bond. R⁹ is an aliphatic hydrocarbon group. These aliphatic hydrocarbon groups may be linear or branched and may have an unsaturated bond in their structure. The aliphatic hydrocarbons groups may have a cyclic ring structure as a partial structure. In structural formula (C-1), R⁸ is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. In structural formula (C-1), R⁹ is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

(iii) In the Case where the Functional Group (I) is an Acetylacetonate Group

In the structural moiety (C) having both an acetylacetonate group and a carbon-carbon unsaturated bond (the functional group (II)), no particular limitation is imposed on the specific structure of the structural moiety (C) excluding the acetylacetonate group and the carbon-carbon unsaturated bond so long as the structural moiety (C) has at least one acetylacetonate group and at least one carbon-carbon unsaturated bond. One example of the specific structure is a group represented by structural formula (C-1) below.

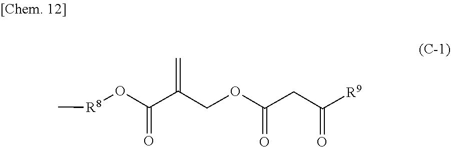

In formula (C-1), R⁸ is an aliphatic hydrocarbon group or a direct bond, and R⁹ is an aliphatic hydrocarbon group.

In structural formula (C-1), R⁸ is an aliphatic hydrocarbon group or a direct bond. R⁹ is an aliphatic hydrocarbon group. These aliphatic hydrocarbon groups may be linear or branched and may have an unsaturated bond in their structure. The aliphatic hydrocarbon groups may have a cyclic ring structure as a partial structure. In structural formula (C-1), R⁸ is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. In structural formula (C-1), R⁹ is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

(iv) In the Case where the Functional Group (I) is an Oxalate Group

In the structural moiety (C) having both an oxalate group and a carbon-carbon unsaturated bond (the functional group (II)), no particular limitation is imposed on the specific structure of the structural moiety (C) excluding the oxalate group and the carbon-carbon unsaturated bond so long as the structural moiety (C) has at least one oxalate group and at least one carbon-carbon unsaturated bond. One example of the specific structure is a group represented by structural formula (C-1) below.

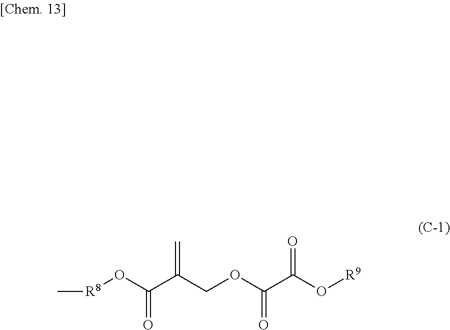

In formula (C-1), R⁸ is an aliphatic hydrocarbon group or a direct bond, and R⁹ is an aliphatic hydrocarbon group.

In structural formula (C-1), R⁹ is an aliphatic hydrocarbon group or a direct bond. R⁹ is an aliphatic hydrocarbon group. These aliphatic hydrocarbon groups may be linear or branched and may have an unsaturated bond in their structure. The aliphatic hydrocarbon groups may have a cyclic ring structure as a partial structure. In structural formula (C-1), R⁸ is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. In structural formula (C-1), R⁹ is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

(v) In the Case where the Functional Group (I) is a Malonate

In the structural moiety (C) having both a malonate group and a carbon-carbon unsaturated bond (the functional group (II)), no particular limitation is imposed on the specific structure of the structural moiety (C) excluding the malonate group and the carbon-carbon unsaturated bond so long as the structural moiety (C) has at least one malonate group and at least one carbon-carbon unsaturated bond. One example of the specific structure is a group represented by structural formula (C-1) below.

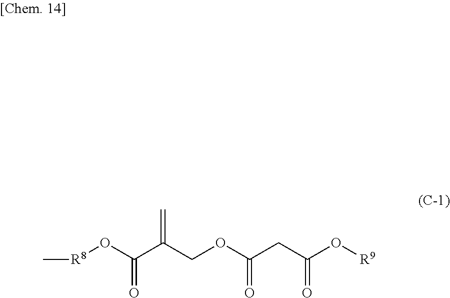

In formula (C-1), R⁸ is an aliphatic hydrocarbon group or a direct bond, and R⁹ is an aliphatic hydrocarbon group.

In structural formula (C-1), R⁹ is an aliphatic hydrocarbon group or a direct bond. R⁹ is an aliphatic hydrocarbon group. These aliphatic hydrocarbon groups may be linear or branched and may have an unsaturated bond in their structure. The aliphatic hydrocarbon groups may have a cyclic ring structure as a partial structure. In structural formula (C-1), R⁸ is preferably an alkanediyl group and more preferably a linear alkanediyl group because better properties such as adhesion to a substrate are obtained while the heat resistance and robustness of the calixarene compound are maintained. The number of carbon atoms in R⁸ is preferably in the range of 1 to 12 and more preferably in the range of 1 to 6. In structural formula (C-1), R⁹ is preferably an alkyl group and more preferably a linear alkyl group because better properties such as adhesion to a substrate are obtained while the heat resistance and robustness of the calixarene compound are maintained. The number of carbon atoms in R⁹ is preferably in the range of 1 to 12 and more preferably in the range of 1 to 6.

<Organic Group (D)>

No particular limitation is imposed on the monovalent organic group (D) that has 1 to 20 carbon atoms and is other than the structural moieties (A), (B), and (C). Examples of the monovalent organic group (D) include aliphatic hydrocarbon groups and groups obtained by substituting one or a plurality of hydrogen atoms in aliphatic hydrocarbon groups with one or a plurality of halogen atoms. The aliphatic hydrocarbon groups may be linear or branched. The aliphatic hydrocarbon groups may have a cyclic ring structure as a partial structure. In particular, the organic group (D) is preferably an aliphatic hydrocarbon group, more preferably an alkyl group, and particularly preferably a linear alkyl group because better properties such as adhesion to a substrate are obtained while the heat resistance and robustness of the calixarene compound are maintained. The number of carbon atoms in the organic group (D) is more preferably in the range of 4 to 20 and particularly preferably in the range of 5 to 20.

In the calixarene compound in the present embodiment, no particular limitation is imposed on the combination of R¹s and R²s so long as at least one functional group (I) and at least one carbon-carbon unsaturated bond are present in one molecule. Specifically, for example, in the case where the functional group (I) is a cyano group, an acetylacetonate group, an oxalate group, or a malonate group, when at least one of R¹s and R²s in one molecule is the structural moiety (C), no particular limitation is imposed on the rest of R¹s and R²s. Moreover, in the case where the functional group (I) is a cyano group, an acetylacetonate group, an oxalate group, or a malonate group, when at least one of R¹s and R²s in one molecule is the structural moiety (A) and at least another one is the structural moiety (B), no particular limitation is imposed on the rest of R²s and R²s. For example, in the case where the functional group (I) is a maleate group, when at least one of R¹s and R²s in one molecule is the structural moiety (A) or the structural moiety (C), no particular limitation is imposed on the rest of R¹s and R²s.

A compound in which all R²s in one molecule are hydrogen atoms (E) is not included in the calixarene compound in the present embodiment.

In structural formula (1), R³s are each independently a hydrogen atom, an aliphatic hydrocarbon group optionally having a substituent, or an aryl group optionally having a substituent. Some specific examples of R³ include: aliphatic hydrocarbon groups such as alkyl groups (e.g., a methyl group, an ethyl group, a propyl group, an isopropyl group, a butyl group, a t-butyl group, a pentyl group, a hexyl group, a cyclohexyl group, a heptyl group, an octyl group, and a nonyl group); groups obtained by substituting one or a plurality of hydrogen atoms in aliphatic hydrocarbon groups with one or a plurality of hydroxy groups, alkoxy groups, halogen atoms, etc.; aromatic ring-containing hydrocarbon groups such as a phenyl group, a tolyl group, a xylyl group, a naphthyl group, and an anthryl group; and aromatic ring-containing hydrocarbon groups with their aromatic ring substituted with a hydroxy group, an alkyl group, an alkoxy group, a halogen atom, etc. In particular, R³s are each preferably a hydrogen atom.

In structural formula (1), no particular limitation is imposed on the positions of the points of attachment indicated by *s. In particular, a compound represented by structural formula (1-1) or (1-2) below is preferred from the viewpoint of production advantages and because better properties such as adhesion to a substrate are obtained while the heat resistance and robustness of the calixarene compound are maintained. In these compounds represented by these structural formulas, functional groups having conflicting properties such as a hydrophobic functional group and a hydrophilic functional group or a reactive functional group and a nonreactive functional group are attached to the benzene ring so as to be arranged in opposite directions. The compound having this configuration is industrially more advantageous because the surface functionality of a cured product to be obtained can be improved significantly while adhesion to a substrate is maintained.

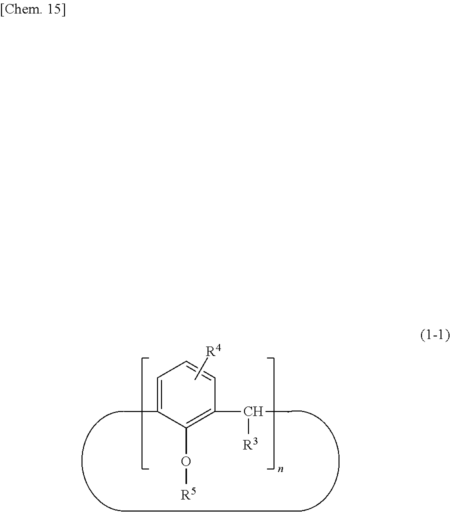

In formula (1-1),

R³ and n are the same as described above;

R⁴ represents a monovalent organic group (d1) having 1 to 20 carbon atoms and represented by —X—R (X is a direct bond or a carbonyl group, and R is a hydrogen atom or an aliphatic hydrocarbon group having 1 to 20 carbon atoms); and

R⁵ represents the structural moiety (A), the structural moiety (B), the structural moiety (C), or a hydrogen atom (E) (a compound in which all R⁵s are each a hydrogen atom (E) is excluded).

A plurality of R³s may be the same or different; a plurality of R⁴s may be the same or different; and a plurality of R⁵s may be the same or different.

When the functional group (I) is a cyano group, an acetylacetonate group, an oxalate group, or a malonate group, at least one of the plurality of R⁵s is the structural moiety (C), or at least one of the plurality of R⁵s is the structural moiety (A) and at least another one of the plurality of R⁵s is the structural moiety (B).

When the functional group (I) is a maleate group, at least one of the plurality of R⁵s is the structural moiety (A) or the structural moiety (C).

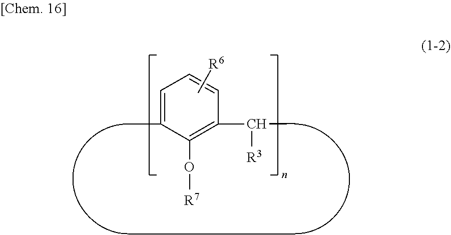

In formula (1-2),

R³ and n are the same as described above;

R⁶ represents the structural moiety (A), the structural moiety (B), or the structural moiety (C); and

R⁷ represents an aliphatic hydrocarbon group having 1 to 20 carbon atoms (d2).

When the functional group (I) is a cyano group, an acetylacetonate group, an oxalate group, or a malonate group, at least one of a plurality of R⁶s is the structural moiety (C), or at least one of the plurality of R⁶s is the structural moiety (A) and at least another one of the plurality of R⁶s is the structural moiety (B).

When the functional group (I) is a maleate group, at least one of the plurality of R⁶s is the structural moiety (A) or the structural moiety (C).

The compound represented by structural formula (1-1) has R⁴s that are relatively hydrophobic functional groups and are located in an upper portion in the structural formula and reactive functional groups in its lower portion. When all R²s in the compound are hydrogen atoms, properties such as adhesion to a substrate are insufficient. It is therefore necessary that at least part of R⁵s be each the structural moiety (A), the structural moiety (B), or the structural moiety (C).

R⁴ in structural formula (1-1) represents the monovalent organic group (d1) represented by —X—R (X is a direct bond or a carbonyl group, and R is a hydrogen atom or an aliphatic hydrocarbon group having 1 to 20 carbon atoms), and the number of carbon atoms in the organic group (d1) is 1 to 20. The aliphatic hydrocarbon in R in the organic group (d1) may be linear or branched and may have a cyclic ring structure as a partial structure. R is preferably a linear alkyl group, and the number of carbon atoms in R is preferably in the range of 4 to 20 and more preferably in the range of 5 to 20. No particular limitation is imposed on the bonding position of R⁴ on the aromatic ring. However, from the point of view that the effects of the invention are more easily obtained and from the point of view of advantages in a production process, the bonding position of R⁴ is particularly preferably the para position relative to the bonding position of —O—R⁵.

R⁵ in structural formula (1-1) is the same as R² described above, and preferred examples of R⁵ are the same as those of R².

The compound represented by structural formula (1-2) has R⁷s that are hydrophobic functional groups and are located in a lower portion in the structural formula and R⁶s that are reactive functional groups and are located in its upper portion.

R⁷ in structural formula (1-2) represents the aliphatic hydrocarbon group (d2) having 1 to 20 carbon atoms. R⁷ may be linear or branched and may have a cyclic ring structure as a partial structure. R⁷ is preferably a linear alkyl group, and the number of carbon atoms in R⁷ is preferably in the range of 4 to 20 and more preferably in the range of 5 to 20.

In structural formula (1-2), R⁶ is the same as R¹ described above, and preferred examples of R⁶ are the same as those of R¹. No particular limitation is imposed on the bonding position of R⁶ on the aromatic ring. However, from the point of view that the effects of the invention are more easily obtained and from the point of view of advantages in a production process, the bonding position of R⁶ is particularly preferably the para position relative to the bonding position of —O—R⁷.

The calixarene compound in the present embodiment may be produced by any method. Examples of the method for producing the calixarene compound in the present embodiment will be described.

To introduce R¹s and R²s in structural formula (1) as substituents, the following method, for example, may be used. Structural moieties corresponding to R¹s are introduced into an intermediate (α) represented by structural formula (2) below:

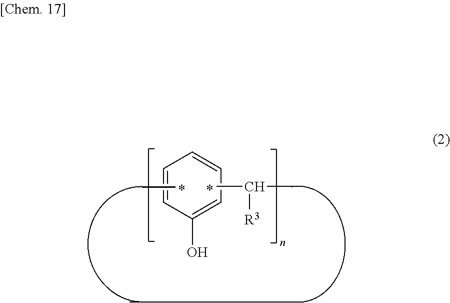

(in formula (2), R³, n, and * are the same as described above). Then part or all of the hydrogen atoms in the phenolic hydroxy groups are each substituted by at least one of the structural moieties (A), (B), (C), and (D) to thereby introduce structural moieties corresponding to R²s. Alternatively, the phenolic hydroxy groups may be first modified to introduce the structural moieties corresponding to R²s, and then the structural moieties corresponding to R¹s may be introduced.

The intermediate (α) represented by structural formula (2) can be produced, for example, by a method using a phenol and an aldehyde compound to directly produce the intermediate (α) or a method including reacting a p-alkylphenol with an aldehyde compound to obtain an intermediate (a) having a calixarene structure and then subjecting the intermediate (a) to a dealkylation reaction in the presence of a phenol and aluminum chloride. In particular, it is preferable to produce the intermediate (α) by the method including reacting a p-alkylphenol with an aldehyde compound to obtain the intermediate (a) having a calixarene structure and then subjecting the intermediate (α) to a dealkylation reaction in the presence of a phenol and aluminum chloride, because the intermediate (α) can be produced with a higher yield.

To introduce the organic groups (D) (e.g., the organic groups (d1)) into the intermediate (α) as R¹s, a method using a Friedel-Crafts alkylation reaction or a method using a Friedel-Crafts acylation reaction to introduce acyl groups may be used. The carbonyl groups in the acyl groups may be reduced to obtain aliphatic hydrocarbon groups. The Friedel-Crafts reactions can be carried out by routine methods. Examples thereof include a method in which the intermediate (α) is reacted with the corresponding halide in the presence of a Lewis acid catalyst such as aluminum chloride. The carbonyl groups can be reduced by a routine method such as a Wolf-Kishner reduction reaction.

To introduce the structural moieties (A), (B) or (C) as R¹s that are substituents on the aromatic rings, the following method, for example, can be used. First, an intermediate (β) represented by the following structural formula (3):

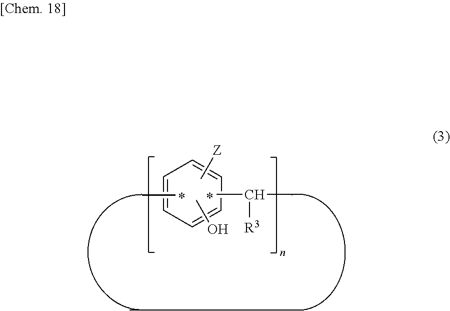

(in formula (3), R³, n, and * are the same as described above, and Z represents a functional group for introduction of R¹) is obtained, and then Z is modified to the structural moiety (A), (B), or (C).

No particular limitation is imposed on Z in the intermediate (β) so long as Z is a functional group that can be converted to the structural moiety (A), (B), or (C). For example, when Z is an allyl group, it is known that an allyl-etherified product of the intermediate (α) undergoes the following rearrangement reaction in the presence of a large excess of an amine compound, and the target intermediate (β) can be obtained with high efficiency.

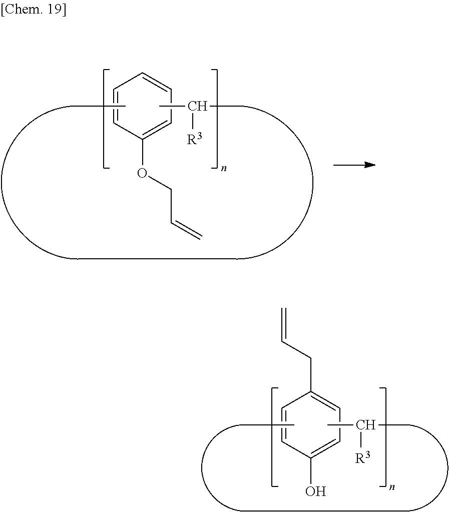

The intermediate (α) can be allyl-etherified by reacting the intermediate (α) with allyl halide under basic catalytic conditions in the same manner as in so-called Williamson ether synthesis. No particular limitation is imposed on the amine compound used for the rearrangement reaction, and examples of the amine compound include: tertiary amines such as N,N-dimethylaniline, N,N-diethylaniline, N,N,N-trimethylamine, N,N,N-triethylamine, and diisopropylethylamine; and secondary amines such as N,N-dimethylamine and N,N-diethylamine. One of them may be used alone, or two or more of them may be used in combination.

No particular limitation is imposed on the method for modifying the allyl groups in the intermediate (β) to the structural moieties (A), (B), or (C). The simplest specific example of the method is a method including epoxidizing the allyl groups and reacting the resulting compound with a carbon-carbon unsaturated bond-containing carboxylic acid compound such as (meth)acrylic acid. Many methods can be used to epoxidize the allyl groups. One example is a method using a peracid such as meta-chloroperoxybenzoic acid or trifluoroperacetic acid.

When Z in the intermediate (β) is a group having a hydroxy group, the intermediate (β) is highly useful because Z can be easily modified to the structural moiety (A), (B), or (C). To obtain the intermediate (β) having hydroxymethyl groups as Zs with a high yield, the following methods may be used. In a method represented by a formula below, the intermediate (α) is halomethylated, and the halomethylated intermediate (α) is reacted with a metal salt of organic carboxylic acid in the presence of a quaternary ammonium salt to thereby subject the halomethylated intermediate (α) to acyloxylation. Then the resulting product is hydroxymethylated through hydrolysis using a metal hydroxide. In another method, the intermediate (α) is formylated, and a reducing agent is used to obtain hydroxymethyl groups.

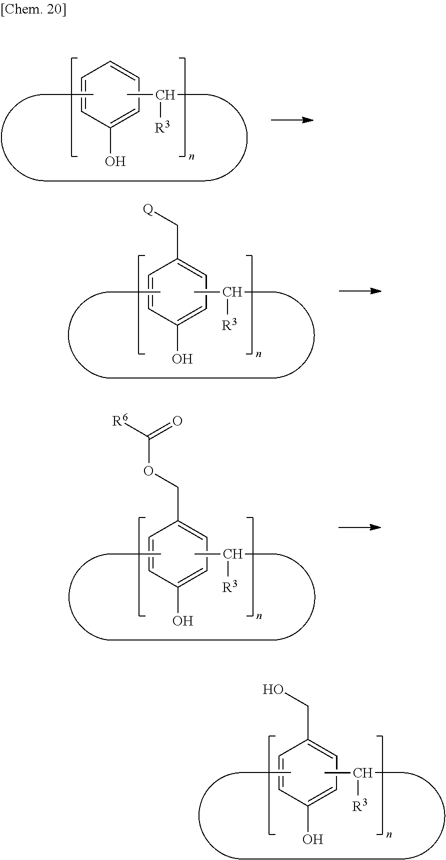

In the above formula, Q represents a halogen atom such as a chlorine atom, a bromine atom, or an iodine atom, and R⁶ represents an alkyl group having 1 to 4 carbon atoms or an alkylene group.

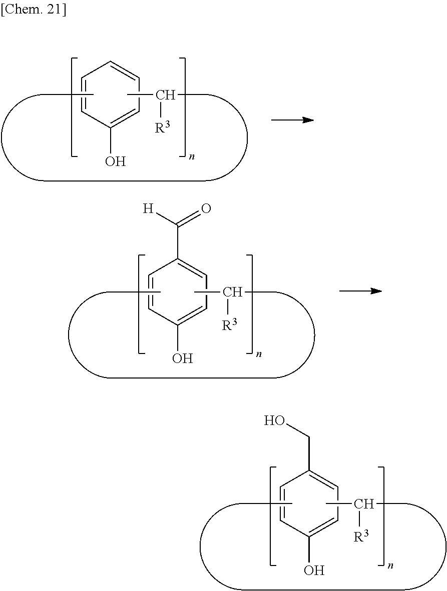

No particular limitation is imposed on the halomethylation method. Examples thereof include a method in which the intermediate (α) is reacted with paraformaldehyde and hydrogen chloride in an acetic acid solvent to chloromethylate the intermediate (α) and a method in which the intermediate (α) is reacted with hydrogen bromide instead of hydrogen chloride under the same conditions to bromomethylate the intermediate (α). No particular limitation is imposed on the quaternary ammonium salt used for the acyloxylation, and examples thereof include tetrabutylammonium bromide, benzyltributylammonium bromide, benzyltrimethylammonium bromide, benzyltributylammonium bromide, tetraethylammonium bromide, benzyltriethylammonium chloride, benzyltrimethylammonium chloride, benzyltributylammonium chloride, tetraethylammonium chloride, methyltributylammonium chloride, and tetrabutylammonium chloride. No particular limitation is imposed on the organic carboxylic acid, and examples thereof include sodium acetate, potassium acetate, sodium propionate, potassium propionate, sodium acrylate, potassium acrylate, sodium methacrylate, and potassium methacrylate.

No particular limitation is imposed on the formylation method. For example, a routine method such as the Vilsmeier-Haack reaction in which the intermediate (α) is reacted with N,N-dimethylformamide and phosphorus oxychloride or the Duff reaction in which the intermediate (α) is formylated using hexamethylenetetramine activated by acid can be used. No particular limitation is imposed on the method for reducing the formylated product obtained. For example, a routine method such as a catalytic reduction method using hydrogen in the presence of a metal hydride such as sodium borohydride or lithium aluminum hydride or a metal catalyst such as palladium can be used.

When Z in the intermediate (β) is a group having a hydroxy group, no particular limitation is imposed on the method for modifying the above group to the structural moiety (A), (B), or (C). Simplest specific examples of the method that can be used include: a method in which a carbon-carbon unsaturated bond-containing carboxylic acid compound such as (meth)acrylic acid is subjected to an esterification reaction with the above hydroxy group under neutral conditions using N,N′-dicyclohexylcarbodiimide or a Mitsunobu reagent containing diethyl azodicarboxylate and triphenylphosphine; and a method in which a carbon-carbon unsaturated bond-containing carboxylic acid halide such as (meth)acrylic acid chloride is subjected to an esterification reaction with the above hydroxy group in the presence of a base.

Examples of the method for converting the hydroxy group in Z to a cyano group include a method that uses acetone cyanohydrin and the Mitsunobu reagent.

Examples of the method that can be used to convert the hydroxy group in Z to a maleate group include: a method in which a carboxylic acid-containing maleic acid monoester compound such as maleic acid monomethyl ester is subjected to an esterification reaction with the hydroxy group under neutral conditions using N,N′-dicyclohexylcarbodiimide or the Mitsunobu reagent containing diethyl azodicarboxylate and triphenylphosphine; and a method in which maleate-containing carboxylic acid halide such as methyl maleinyl chloride is subjected to an esterification reaction with the hydroxy group in the presence of a base.

Examples of the method for converting the hydroxy group in Z to an acetyliacetonate group include a method in which the intermediate (β) is reacted with a diketene-acetone adduct (2,2,6-trimethyl-1,3-dioxin-4-one) under heating conditions.

Examples of the method that can be used to convert the hydroxy group in Z to an oxalate group include a method in which oxalate-containing carboxylic acid halide such as methyl oxal chloride is subjected to an esterification reaction with the hydroxy group in the presence of a base.

Examples of the method that can be used to convert the hydroxy group in Z to a malonate group include: a method in which a carboxylic acid-containing malonic acid monoester compound such as malonic acid monomethyl ester is subjected to an esterification reaction with the hydroxy group under neutral conditions using N,N′-dicyclohexylcarbodiimide or the Mitsunobu reagent containing diethyl azodicarboxylate and triphenylphosphine; and a method in which a malonate-containing carboxylic acid halide such as methyl malonyl chloride is subjected to an esterification reaction with the hydroxy group in the presence of a base.

When the Z group in the intermediate (s) is a group having a halogenated alkyl group, the intermediate (β) is highly usable because the Z group can be easily substituted with the structural moiety (A). In particular, when Z is a halomethyl group, Z can be easily converted to the structural moiety (A) having a cyano group using a routine method including halomethylating the intermediate (α) by the halomethylation method described above and then reacting the resulting intermediate (α) with sodium cyanide.

No particular limitation is imposed on the method for modifying part or all of the phenolic hydroxy groups to structural moieties corresponding to R²s, and a well-known reaction such as a Mitsunobu reaction for a general phenolic hydroxy group or Williamson ether synthesis can be appropriately used.

The calixarene compound in the present embodiment has, in its molecule, at least one functional group (I) and at least one carbon-carbon unsaturated bond. Examples of the method for obtaining the above compound include: a method including introducing the structural moiety (B) into each of at least one of the phenolic hydroxy groups in the intermediate (α), the intermediate (β), or a compound obtained by introducing R¹ onto each aromatic ring in the intermediate (α) or (β) and then introducing the structural moiety (A) into each of the rest of the phenolic hydroxy groups; a method including introducing a structural moiety having an alcoholic hydroxy group into each of the phenolic hydroxy groups, converting at least one of the alcoholic hydroxy groups to the structural moiety (A), and converting at least another one of the alcoholic hydroxy groups to the structural moiety (B).

Examples of the method for introducing cyano group-containing structural moieties (A) into phenolic hydroxy groups include: a method including reacting the phenolic hydroxy groups with the corresponding cyano group-containing halogenated alkylated compound in the same manner as in the Williamson ether synthesis; a method including converting one of a plurality of halogenated alkylated compounds to a phenol ether in the same manner as in the Williamson ether synthesis and reacting a cyanide of an alkali metal with the halogenated moiety of another one of the halogenated alkylated compounds in the presence of a quaternary ammonium salt; and a method including reacting a halogenated silyl-etherified compound with each phenolic hydroxy group to obtain a phenol ether, followed by subjecting the phenol ether to desilylation in the presence of tetrabutylammonium fluoride, or reacting an appropriate halide with each phenolic hydroxy group to introduce a ketone structure or an ester structure, followed by reducing the resulting group to form an alcoholic hydroxy group, and then cyanating the alcoholic hydroxy group moiety using acetone cyanohydrin and the Mitsunobu reagent.

Examples of the method for introducing maleate group-containing structural moieties (A) into phenolic hydroxy groups include: a method including reacting the phenolic hydroxy groups with the corresponding maleate group-containing halogenated alkylated compound in the same manner as in the Williamson ether synthesis; a method including reacting a halogenated silyl-etherified compound with each phenolic hydroxy group to obtain a phenol ether, followed subjecting the phenol ether to desilylation in the presence of tetrabutylammonium fluoride, or reacting an appropriate halide with each phenolic hydroxy group to introduce a ketone structure or an ester structure, followed by reducing the resulting group to form a hydroxy group, and then subjecting the hydroxy group moiety and a carboxylic acid-containing maleic acid monoester compound such as maleic acid monomethyl ester to an esterification reaction under neutral conditions using N,N′-dicyclohexylcarbodiimide or the Mitsunobu reagent containing diethyl azodicarboxylate and triphenylphosphine; and a method including subjecting the hydroxy groups and a maleate-containing carboxylic acid halide such as methyl maleinyl chloride in the presence of a base.

Examples of the method for introducing acetylacetonate group-containing structural moieties (A) into phenolic hydroxy groups include: a method including reacting the phenolic hydroxy groups with the corresponding acetylacetonate group-containing halogenated alkylated compound in the same manner as in the Williamson ether synthesis; and a method including reacting a halogenated silyl-etherified compound with each phenolic hydroxy group to obtain a phenol ether, followed by subjecting the phenol ether to desilylation in the presence of tetrabutylammonium fluoride, or reacting an appropriate halide with each phenolic hydroxy group to introduce a ketone structure or an ester structure, followed by reducing the resulting group to form an alcoholic hydroxy group, and then reacting the alcoholic hydroxy group moiety with the diketene-acetone adduct (2,2,6-trimethyl-1,3-dioxin-4-one) under heating conditions.

Example of the method for introducing oxalate group-containing structural moieties (A) into phenolic hydroxy groups include: a method including reacting the phenolic hydroxy groups with the corresponding oxalate group-containing halogenated alkylated compound in the same manner as in the Williamson ether synthesis; and a method including reacting a halogenated silyl-etherified compound with each phenolic hydroxy group to obtain a phenol ether, followed by subjecting the phenol ether to desilylation in the presence of tetrabutylammonium fluoride, or reacting an appropriate halide with each phenolic hydroxy group to introduce a ketone structure or an ester structure, followed by reducing the resulting group to form a hydroxy group, and then subjecting the hydroxy group moiety and an oxalate-containing carboxylic acid halide such as methyl oxal chloride to an esterification reaction in the presence of a base.

Examples of the method for introducing malonate group-containing structural moieties (A) into phenolic hydroxy groups include: a method including reacting the phenolic hydroxy groups with the corresponding malonate group-containing halogenated alkylated compound in the same manner as in the Williamson ether synthesis; a method including reacting a halogenated silyl-etherified compound with each phenolic hydroxy group to obtain a phenol ether, followed by subjecting the phenol ether to desilylation in the presence of tetrabutylammonium fluoride, or reacting an appropriate halide with each phenolic hydroxy group to introduce a ketone structure or an ester structure, followed by reducing the resulting group to form a hydroxy group, and then subjecting the hydroxy group moiety and a carboxylic acid-containing malonic acid monoester compound such as malonic acid monomethyl ester to an esterification reaction under neutral conditions using N,N′-dicyclohexylcarbodiimide or the Mitsunobu reagent containing diethyl azodicarboxylate and triphenylphosphine; and a method including subjecting a malonate-containing carboxylic acid halide such as methyl malonyl chloride to an esterification reaction with the hydroxy group in the presence of a base.

When a phenolic hydroxy group is modified to the structural moiety (B), the following methods can be used. In one method, a Mitsunobu reaction using a compound that has both an alcoholic hydroxy group and a carbon-carbon unsaturated bond and corresponds to the structural moiety (B) is used. In another method, a halogenated silyl etherified compound is reacted with the phenolic hydroxy group to obtain a phenol ether, and then the phenol ether is subjected to desilylation in the presence of tetrabutylammonium fluoride. Alternatively, an appropriate halide is reacted with the phenolic hydroxy group to introduce a ketone structure or an ester structure, and the resulting group is reduced to form an alcoholic hydroxy group. Then the hydroxy group and a carbon-carbon unsaturated bond-containing carboxylic acid compound such as (meth)acrylic acid are subjected to an esterification reaction.

Examples of the alcoholic hydroxy group-containing compound include hydroxyethyl (meth)acrylate, hydroxypropyl (meth)acrylate, glycerin di(meth)acrylate, trimethylolpropane dimethacrylate, pentaerythritol tri(meth)acrylate, dipentaerythritol penta(meth)acrylate, hydroxyethyl (meth)acrylamide, hydroxypropyl (meth)acrylamide, hydroxyethyl vinyl ether, and hydroxypropyl vinyl ether. In R²s in structural formula (1), the ratio of the structural moieties (B) and hydrogen atoms (E) can be appropriately controlled by adjusting a reaction molar ratio.

No particular limitation is imposed on the esterification reaction of the alcoholic hydroxy group with a carbon-carbon unsaturated bond-containing carboxylic acid compound such as (meth)acrylic acid. Examples of the method for the esterification reaction include: a method including subjecting the carbon-carbon unsaturated bond-containing carboxylic acid compound such as (meth)acrylic acid to an esterification reaction with the alcoholic hydroxy group formed by the reduction described above under neutral conditions using N,N′-dicyclohexylcarbodiimide or the Mitsunobu reagent containing diethyl azodicarboxylate and triphenylphosphine; and a method including subjecting a carbon-carbon unsaturated bond-containing carboxylic acid halide such as (meth)acrylic acid chloride to an esterification reaction with the alcoholic hydroxy group formed by the reduction described above in the presence of a base.

When R²s in structural formula (1) are structural moieties (C) each having both a cyano group and a carbon-carbon unsaturated bond, the following methods, for example, can be used. In one method, a halide corresponding to the structural moieties (C) is reacted with part or all of the phenolic hydroxy groups in the intermediate (α), the intermediate (β), or a compound obtained by introducing R¹s into the aromatic rings in one of these intermediates. In another method, structural moieties each having a carbon-carbon unsaturated bond and a silyl ether group are introduced into part or all of the phenolic hydroxy groups, and the resulting product is subjected to desilylation. Then the hydroxy groups generated are cyanated using the acetone cyanohydrin described above and the Mitsunobu reagent described above.

When R²s in structural formula (1) are structural moieties (C) each having both an acetylacetonate group and a carbon-carbon unsaturated bond, the following methods, for example, can be used. In one method, a halide corresponding to the structural moieties (C) is reacted with part or all of the phenolic hydroxy groups in the intermediate (α), the intermediate (β), or a compound obtained by introducing R¹s into the aromatic rings in one of these intermediates. In another method, structural moieties each having a carbon-carbon unsaturated bond and a silyl ether group are introduced into part or all of the phenolic hydroxy groups, and the resulting product is subjected to desilylation. Then the alcoholic hydroxy groups generated are reacted with the diketene-acetone adduct (2,2,6-trimethyl-1,3-dioxin-4-one) described above under heating conditions.

When R²s in structural formula (1) are structural moieties (C) each having both an oxalate group and a carbon-carbon unsaturated bond, the following methods, for example, can be used. In one method, a halide corresponding to the structural moieties (C) is reacted with part or all of the phenolic hydroxy groups in the intermediate (α), the intermediate (β), or a compound obtained by introducing R¹s into the aromatic rings in one of these intermediates. In another method, structural moieties each having a carbon-carbon unsaturated bond and a silyl ether group are introduced into part or all of the phenolic hydroxy groups, and the resulting product is subjected to desilylation. Then the alcoholic hydroxy groups generated and the above-described oxalate-containing carboxylic acid halide such as methyl oxal chloride are subjected to an esterification reaction in the presence of a base.

When R²s in structural formula (1) are structural moieties (C) each having both a malonate group and a carbon-carbon unsaturated bond, the following methods, for example, can be used. In one method, a halide corresponding to the structural moieties (C) is reacted with part or all of the phenolic hydroxy groups in the intermediate (α), the intermediate (β), or a compound obtained by introducing R¹s into the aromatic rings in one of these intermediates. In another method, structural moieties each having a carbon-carbon unsaturated bond and a silyl ether group are introduced into part or all of the phenolic hydroxy groups, and the resulting product is subjected to desilylation. Then the alcoholic hydroxy groups generated and the above-describe carboxylic acid-containing malonic acid monoester compound such as malonic acid monomethyl ester are subjected to an esterification reaction under neutral conditions using N,N′-dicyclohexylcarbodiimide or the Mitsunobu reagent containing diethyl azodicarboxylate and triphenylphosphine. In another method, a malonate-containing carboxylic acid halide such as methyl malonyl chloride is subjected to an esterification reaction in the presence of a base.

Examples of the method for introducing an aliphatic hydrocarbon group (d2) having 1 to 20 carbon atoms and serving as the organic group (D) into a phenolic hydroxy group include a method including reacting the phenolic hydroxy group with a halide of the corresponding aliphatic hydrocarbon under basic catalysis conditions in the same manner as in the so-called Williamson ether synthesis.

The method for producing the calixarene compound in the present embodiment has been described by way of some specific examples. However, the calixarene compound in the present embodiment is not limited to those obtained by the above specific production methods. For example, by combining the above-exemplified elementary reactions appropriately or using some of them repeatedly, calixarene compounds having various and complex structures can be obtained.

The calixarene compound in the present embodiment is excellent in properties such as adhesion to a substrate and toughness, which are problems in conventional calixarene compounds, while the characteristic properties of the calixarene compound such as high heat resistance and high hardness are maintained. No particular limitation is imposed on the applications of the calixarene compound in the present embodiment, and the calixarene compound can be used for a wide variety of applications. Part of the applications are exemplified below.

The calixarene compound in the present embodiment contains, in its molecule, at least one carbon-carbon unsaturated bond. Therefore, the calixarene compound can be used as a curable resin material with the carbon-carbon unsaturated bond serving as a polymerizable group. The mode of curing may be photocurable or thermosetting. In the following description, the calixarene compound is used as a photocurable compound.

When the calixarene compound in the present embodiment is used as a photocurable resin material, it is preferable that a photopolymerization initiator described later, an additional photocurable composition, various additives, etc. are added to prepare a curable composition. Examples of the additional photocurable compound include compounds having a (meth)acryloyl group. Examples of the compound having a (meth)acryloyl group include: mono(meth)acrylate compounds and modified products (R1) thereof; aliphatic hydrocarbon poly(meth)acrylate compounds and modified products (R2) thereof; alicyclic poly(meth)acrylate compounds and modified products (R3) thereof; aromatic poly(meth)acrylate compounds and modified products (R4) thereof; silicone chain-containing (meth)acrylate resins and modified products (R5) thereof; epoxy (meth)acrylate resins and modified products (R6) thereof; urethane (meth)acrylate resins and modified products (R7) thereof; acrylic (meth)acrylate resins and modified products (R8) thereof; and dendrimer-type (meth)acrylate resins and modified products (R9) thereof.

Examples of the mono(meth)acrylate compounds and modified products (R1) thereof include: aliphatic mono(meth)acrylate compounds such as methyl (meth)acrylate, ethyl (meth)acrylate, hydroxyethyl (meth)acrylate, propyl (meth)acrylate, hydroxypropyl (meth)acrylate, butyl (meth)acrylate, and 2-ethylhexyl (meth)acrylate; alicyclic mono(meth)acrylate compounds such as cyclohexyl (meth)acrylate, isobornyl (meth)acrylate, and adamantyl mono(meth)acrylate; heterocyclic mono(meth)acrylate compounds such as glycidyl (meth)acrylate and tetrahydrofurfuryl acrylate; aromatic mono(meth)acrylate compounds such as phenyl (meth)acrylate, benzyl (meth)acrylate, phenoxy (meth)acrylate, phenoxyethyl (meth)acrylate, phenoxyethoxyethyl (meth)acrylate, 2-hydroxy-3-phenoxypropyl (meth)acrylate, phenylphenol (meth)acrylate, phenylbenzyl (meth)acrylate, phenoxybenzyl (meth)acrylate, benzylbenzyl (meth)acrylate, phenylphenoxyethyl (meth)acrylate, and p-cumylphenol (meth)acrylate; mono(meth)acrylate compounds such as a compound represented by the following structural formula (5):

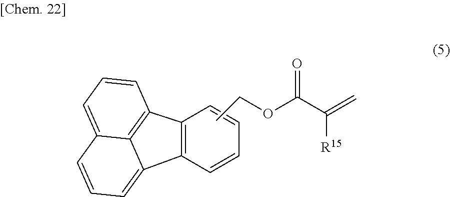

(in formula (5), R¹⁵ is a hydrogen atom or a methyl group); (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various mono(meth)acrylate compounds; and lactone-modified products obtained by introducing a (poly)lactone structure into the molecular structures of the above various mono(meth)acrylate compounds.

Examples of the aliphatic hydrocarbon poly(meth)acrylate compounds and modified products (R2) thereof include: aliphatic di(meth)acrylate compounds such as ethylene glycol di(meth)acrylate, propylene glycol di(meth)acrylate, butanediol di(meth)acrylate, hexanediol di(meth)acrylate, and neopentyl glycol di(meth)acrylate; aliphatic tri(meth)acrylate compounds such as trimethylolpropane tri(meth)acrylate, glycerin tri(meth)acrylate, pentaerythritol tri(meth)acrylate, ditrimethylolpropane tri(meth)acrylate, and dipentaerythritol tri(meth)acrylate; tetrafunctional and higher functional aliphatic poly(meth)acrylate compounds such as pentaerythritol tetra(meth)acrylate, ditrimethylolpropane tetra(meth)acrylate, dipentaerythritol tetra(meth)acrylate, dipentaerythritol penta(meth)acrylate, and dipentaerythritol hexa(meth)acrylate; (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various aliphatic hydrocarbon poly(meth)acrylate compounds; and lactone-modified products obtained by introducing a (poly)lactone structure into the molecular structures of the above various aliphatic hydrocarbon poly(meth)acrylate compounds.

Examples of the alicyclic poly(meth)acrylate compounds and modified products (R3) thereof include: alicyclic di(meth)acrylate compounds such as 1,4-cyclohexanedimethanol di(meth)acrylate, norbornane di(meth)acrylate, norbornane dimethanol di(meth)acrylate, dicyclopentanyl di(meth)acrylate, and tricyclodecane dimethanol di(meth)acrylate; (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various alicyclic poly(meth)acrylate compounds; and lactone-modified products obtained by introducing a (poly)lactone structure into the molecular structures of the above various alicyclic poly(meth)acrylate compounds.

Examples of the aromatic poly(meth)acrylate compounds and modified products (R4) thereof include: aromatic di(meth)acrylate compounds such as biphenol di(meth)acrylate, bisphenol di(meth)acrylate, a bicarbazole compound represented by structural formula (9) below:

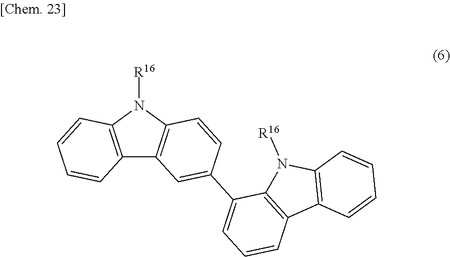

(in formula (6), R¹⁶s are each independently a (meth)acryloyl group, a (meth)acryloyloxy group, or a (meth)acryloyloxyalkyl group), fluorene compounds represented by structural formulas (7-1) and (7-2):

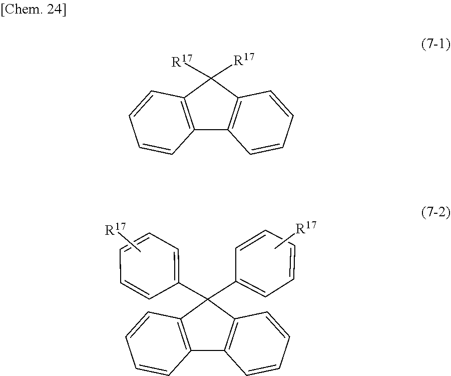

(in formulas (7-1) and (7-2), R¹⁷s are each independently a (meth)acryloyl group, a (meth)acryloyloxy group, or a (meth)acryloyloxyalkyl group); (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various aromatic poly(meth)acrylate compounds; and lactone-modified products obtained by introducing a (poly)lactone structure into the molecular structures of the above various aromatic poly(meth)acrylate compounds.

No particular limitation is imposed on the silicone chain-containing (meth)acrylate resins and modified products (R5) thereof so long as they are compounds each having a silicone chain and a (meth)acryloyl group in their molecule, and various compounds can be used. No particular limitation is imposed on the method for producing these resins. Specific examples of the silicone chain-containing (meth)acrylate resins and modified products (R5) thereof include a reaction product of a silicone compound having an alkoxysilane group and a hydroxy group-containing (meth)acrylate compound.

Examples of commercial products of the alkoxysilane group-containing silicone compound include “X-40-9246” (alkoxy group content: 12% by weight), “KR-9218” (alkoxy group content: 15% by weight), “X-40-9227” (alkoxy group content: 15% by weight), “KR-510” (alkoxy group content: 17% by weight), “KR-213” (alkoxy group content: 20% by weight), “X-40-9225” (alkoxy group content: 24% by weight), “X-40-9250” (alkoxy group content: 25% by weight), “KR-500” (alkoxy group content: 28% by weight), “KR-401N” (alkoxy group content: 33% by weight), “KR-515” (alkoxy group content: 40% by weight), and “KC-89S” (alkoxy group content: 45% by weight) that are manufactured by Shin-Etsu Chemical Co., Ltd. One of them may be used alone, or two or more of them may be used in combination. In particular, the alkoxy group content is preferably in the range of 15 to 40% by weight. When two or more silicone compounds are used in combination, it is preferable that the average of their alkoxy group contents is in the range of 15 to 40% by weight.

Examples of the hydroxy group-containing (meth)acrylate compound include: hydroxy group-containing (meth)acrylate compounds such as hydroxyethyl (meth)acrylate, hydroxypropyl (meth)acrylate, trimethylolpropane di(meth)acrylate, pentaerythritol tri(meth)acrylate, ditrimethylolpropane tri(meth)acrylate, and dipentaerythritol penta(meth)acrylate; (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various hydroxy group-containing (meth)acrylate compounds; and lactone-modified products obtained by introducing a (poly)lactone structure into the molecular structures of the above various hydroxy group-containing (meth)acrylate compounds.

Commercial products of the silicone chain-containing (meth)acrylate resins and modified products (R5) thereof may be used, and examples of the commercial products include: silicone oils each having a (meth)acryloyl group at one end such as “X-22-174ASX” (methacryloyl group equivalent: 900 g/eq), “X-22-174BX” (methacryloyl group equivalent: 2,300 g/eq), “X-22-174DX” (methacryloyl group equivalent: 4,600 g/eq), “KF-2012” (methacryloyl group equivalent: 4,600 g/eq), “X-22-2426” (methacryloyl group equivalent: 12,000 g/eq), “X-22-2404” (methacryloyl group equivalent: 420 g/eq), and “X-22-2475” (methacryloyl group equivalent: 420 g/eq) that are manufactured by Shin-Etsu Chemical Co., Ltd.; silicone oils each having (meth)acryloyl groups at both ends such as “X-22-164” (methacryloyl group equivalent: 190 g/eq), “X-22-164AS” (methacryloyl group equivalent: 450 g/eq), “X-22-164A” (methacryloyl group equivalent: 860 g/eq), “X-22-164B” (methacryloyl group equivalent: 1,600 g/eq), “X-22-164C” (methacryloyl group equivalent: 2,400 g/eq), “X-22-164E” (methacryloyl group equivalent: 3,900 g/eq), and “X-22-2445” (acryloyl group equivalent: 1,600 g/eq) that are manufactured by Shin-Etsu Chemical Co., Ltd.; oligomer-type silicone compounds having a plurality of (meth)acryloyl groups in their molecule such as “KR-513” (methacryloyl group equivalent: 210 g/eq), “−40-9296” (methacryloyl group equivalent: 230 g/eq) that are manufactured by Shin-Etsu Chemical Co., Ltd.; and “AC-SQ TA-100” (acryloyl group equivalent: 165 g/eq), “AC-SQ SI-20” (acryloyl group equivalent: 207 g/eq), “MAC-SQ TM-100” (methacryloyl group equivalent: 179 g/eq), “MAC-SQ SI-20” (methacryloyl group equivalent: 224 g/eq), and “MAC-SQ HDM” (methacryloyl group equivalent: 239 g/eq) that are manufactured by TOAGOSEI Co., Ltd.

The weight average molecular weights (Mw) of the silicone chain-containing (meth)acrylate resins and modified products (R5) thereof are preferably in the range of 1,000 to 10,000 and more preferably in the range of 1,000 to 5,000. Their (meth)acryloyl group equivalent is preferably in the range of 150 to 5,000 g/eq and more preferably in the range of 150 to 2,500 g/eq.

Examples of the epoxy (meth)acrylate resins and modified products (R6) thereof include a compound obtained by reacting (meth)acrylic acid or an anhydride thereof with an epoxy resin. Examples of the epoxy resin include: diglycidyl ethers of dihydric phenols such as hydroquinone and catechol; diglycidyl ethers of biphenol compounds such as 3,3′-biphenyldiol and 4,4′-biphenyldiol; bisphenol-type epoxy resins such as bisphenol A-type epoxy resins, bisphenol B-type epoxy resins, bisphenol F-type epoxy resins, and bisphenol S-type epoxy resins; polyglycidyl ethers of naphthol compounds such as 1,4-naphthalenediol, 1,5-naphthalenediol, 1,6-naphthalenediol, 2,6-naphthalenediol, 2,7-naphthalenediol, binaphthol, and bis(2,7-dihydroxynaphthyl)methane; triglycidyl ethers such as 4,4′,4″-methylidynetrisphenol; novolac-type epoxy resins such as phenol novolac-type epoxy resins and cresol novolac resins; (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various epoxy resins; and lactone-modified products obtained by introducing a (poly)lactone structure into the molecular structures of the above various epoxy resins.

Examples of the urethane (meth)acrylate resins and modified products (R⁷) thereof include compounds obtained by reacting various polyisocyanate compounds with various hydroxy group-containing (meth)acrylate compounds and various optional polyol compounds. Examples of the polyisocyanate compounds include: aliphatic diisocyanate compounds such as butane diisocyanate, hexamethylene diisocyanate, 2,2,4-trimethylhexamethylene diisocyanate, and 2,4,4-trimethylhexamethylene diisocyanate; alicyclic diisocyanate compounds such as norbornane diisocyanate, isophorone diisocyanate, hydrogenated xylylene diisocyanate, and hydrogenated diphenylmethane diisocyanate; aromatic diisocyanate compounds such as tolylene diisocyanate, xylylene diisocyanate, tetramethylxylylene diisocyanate, diphenylmethane diisocyanate, and 1,5-naphthalene diisocyanate; a polymethylene polyphenyl polyisocyanate having a repeating structure represented by structural formula (8) below:

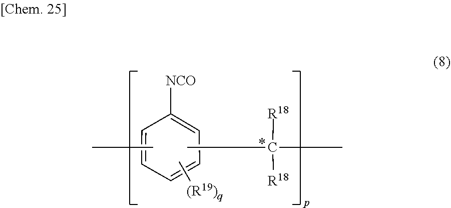

(in formula (8), R¹⁸s are each independently a hydrogen atom or a hydrocarbon group having 1 to E carbon atoms; R¹⁹s are each independently an alkyl group having 1 to 4 carbon atoms or the point of attachment to the structural moiety represented by structural formula (8) through a methylene group marked with an *; q is an integer of 0 or 1 to 3, and p is an integer of 1 or more); isocyanurate-modified products thereof; biuret-modified products thereof; and allophanate modified products thereof.

Examples of the hydroxy group-containing (meth)acrylate compound include: hydroxy group-containing (meth)acrylate compounds such as hydroxyethyl (meth)acrylate, hydroxypropyl (meth)acrylate, trimethylolpropane di(meth)acrylate, pentaerythritol tri(meth)acrylate, ditrimethylolpropane tri(meth)acrylate, and dipentaerythritol penta(meth)acrylate; (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various hydroxy group-containing (meth)acrylate compounds; and lactone-modified products obtained by introducing a (poly)lactone structure into the molecular structures of the above various hydroxy group-containing (meth)acrylate compounds.

Examples of the polyol compound include: aliphatic polyol compounds such as ethylene glycol, proplene glycol, butanediol, hexanediol, glycerin, trimethylolpropane, ditrimethylolpropane, pentaerythritol, and dipentaerythritol; aromatic polyol compounds such as biphenol and bisphenol; (poly)oxyalkylene-modified products obtained by introducing a (poly)oxyalkylene chain such as a (poly)oxyethylene chain, a (poly)oxypropylene chain, or a (poly)oxytetramethylene chain into the molecular structures of the above various polyol compounds; and lactone-modified products obtained by introducing a (poly)lactone structure into the molecular structures of the above various polyol compounds.

Examples of the acrylic (meth)acrylate resins and modified products (R8) thereof include: a compound obtained by reacting an acrylic resin intermediate obtained by polymerizing a (meth)acrylate monomer (a) used as an essential component and having a reactive functional group such as a hydroxy group, a carboxy group, an isocyanate group, or a glycidyl group with a (meth)acrylate monomer (β) having a reactive functional group that can react with the above functional group to thereby introduce a (meth)acryloyl group.

Examples of the (meth)acrylate monomer (a) having a reactive functional group include: hydroxy group-containing (meth)acrylate monomers such as hydroxyethyl (meth)acrylate and hydroxypropyl (meth)acrylate; carboxy group-containing (meth)acrylate monomers such as (meth)acrylic acid; isocyanate group-containing (meth)acrylate monomers such as 2-acryloyloxyethyl isocyanate, 2-methacryloyloxyethyl isocyanate, and 1,1-bis(acryloyloxymethyl)ethyl isocyanate; and glycidyl group-containing (meth)acrylate monomers such as glycidyl (meth)acrylate and 4-hydroxybutyl acrylate glycidyl ether. One of them may be used alone, or two or more may be used in combination.

The acrylic resin intermediate may be prepared by copolymerizing the (meth)acrylate monomer (a) and an optional additional polymerizable unsaturated group-containing compound. Examples of the additional polymerizable unsaturated group-containing compound include: (meth)acrylic acid alkyl esters such as methyl (meth)acrylate, ethyl (meth)acrylate, propyl (meth)acrylate, butyl (meth)acrylate, and 2-ethylhexyl (meth)acrylate; cyclic ring-containing (meth)acrylates such as cyclohexyl (meth)acrylate, isobornyl (meth)acrylate, and dicyclopentanyl(meth)acrylate; aromatic ring-containing (meth)acrylates such as phenyl (meth)acrylate, benzyl (meth)acrylate, and phenoxyethyl acrylate; silyl group-containing (meth)acrylates such as 3-methacryloxypropyltrimethoxysilane; and styrene derivatives such as styrene, α-methylstyrene, and chlorostyrene. One of them may be used alone, or two or more may be used in combination.

No particular limitation is imposed on the (meth)acrylate monomer (β) so long as it can react with the reactive functional group included in the (meth)acrylate monomer (α). However, from the viewpoint of reactivity, the following combinations are preferred. When the hydroxy group-containing (meth)acrylate is used as the (meth)acrylate monomer (α), it is preferable to use the isocyanate group-containing (meth)acrylate as the (meth)acrylate monomer (β). When the carboxy group-containing (meth)acrylate is used as the (meth)acrylate monomer (α), it is preferable to use the glycidyl group-containing (meth)acrylate as the (meth)acrylate monomer (β). When the isocyanate group-containing (meth)acrylate is used as the (meth)acrylate monomer (α), it is preferable to use the hydroxy group-containing (meth)acrylate as the (meth)acrylate monomer (β). When the glycidyl group-containing (meth)acrylate is used as the (meth)acrylate monomer (α), it is preferable to use the carboxy group-containing (meth)acrylate as the (meth)acrylate monomer (β).

The weight average molecular weights (Mw) of the acrylic (meth)acrylate resins and modified products (R8) thereof are preferably in the range of 5,000 to 50,000. Their (meth)acryloyl group equivalent is preferably in the range of 200 to 300 g/eq.

The dendrimer-type (meth)acrylate resins and modified products (R9) thereof are resins having a highly branched regular structure and having (meth)acryloyl groups at ends of the branched chains. The dendrimer-type resins are referred to also as hyperbranched-type resins and star polymers. Examples of such compounds include, but not limited to, compounds represented by structural formulas (9-1) to (9-8) below. Any compound having a highly branched regular structure and having (meth)acryloyl group at ends of the branched chains can be used.

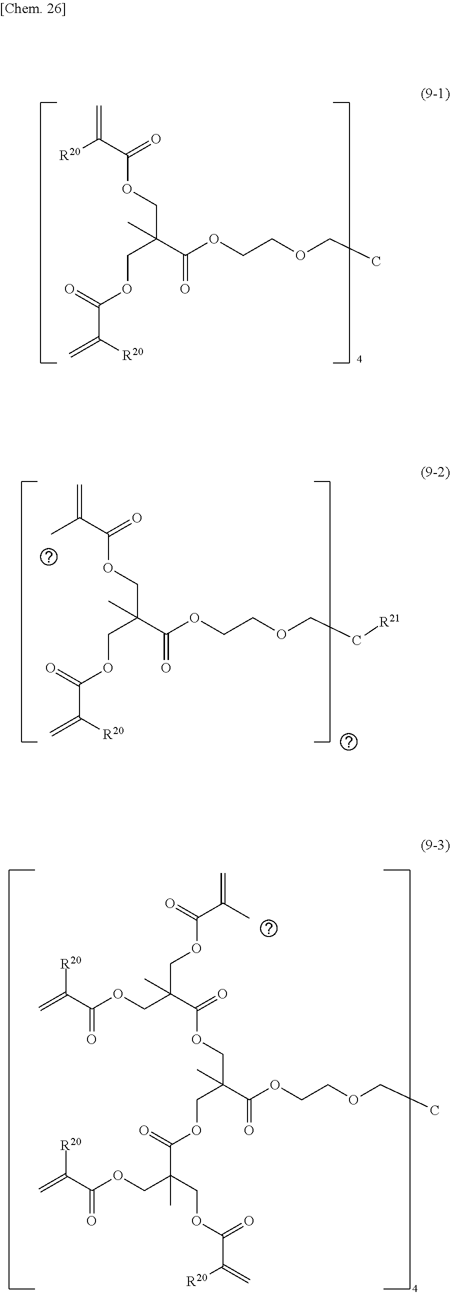

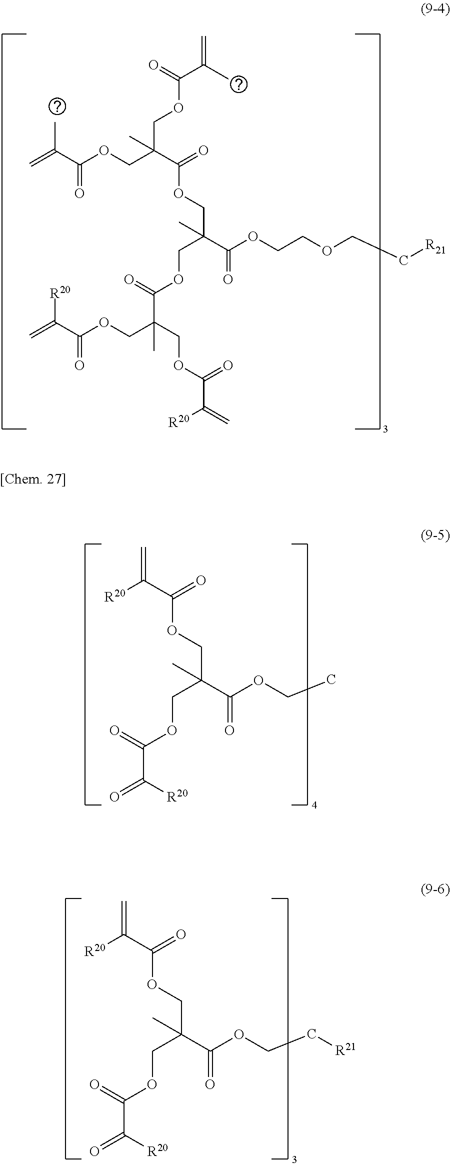

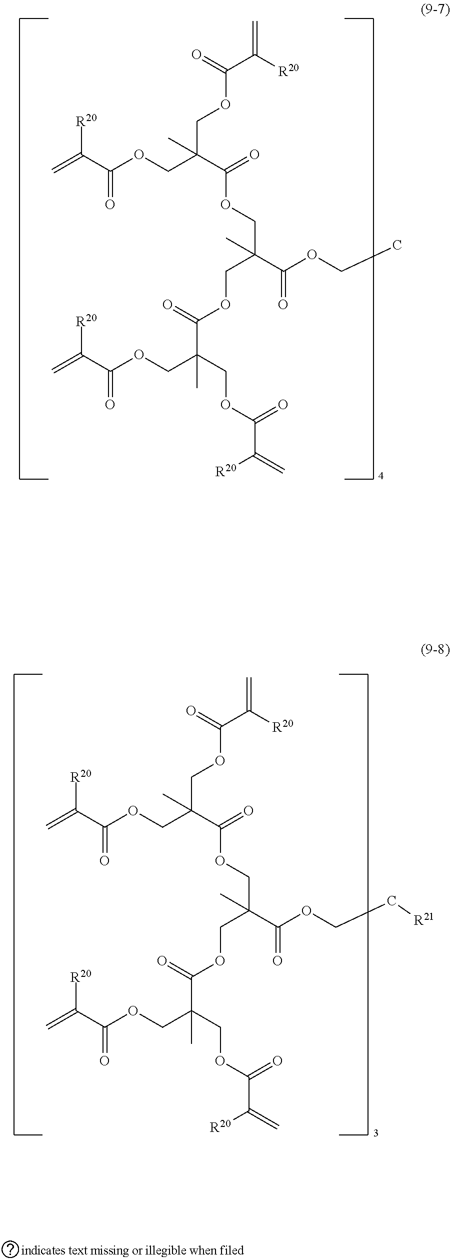

In formulas (9-1) to (9-8), R²⁰ represents a hydrogen atom or a methyl group, and R² represents a hydrocarbon group having 1 to 4 carbon atoms.

Commercial products may be used as the dendrimer-type (meth)acrylate resins and modified products (R9) thereof, and examples of the commercial products include: “Viscoat #1000” [weight average molecular weight (Mw): 1,500 to 2,000, average number of (meth)acryloyl groups per molecule: 14], “Viscoat 1020” [weight average molecular weight (Mw): 1,000 to 3,000], and “SIRIUS 501” [weight average molecular weight (Mw): 15,000 to 23,000] that are manufactured by OSAKA ORGANIC CHEMICAL INDUSTRY LTD.; “SP-1106” [weight average molecular weight (Mw): 1,630, average number of (meth)acryloyl groups per molecule: 18] manufactured by MIWON; “CN2301,” “CN2302” [average number of (meth)acryloyl groups per molecule: 16], “CN2303” [average number of (meth)acryloyl groups per molecule: 6], and “CN2304” [average number of (meth)acryloyl groups per molecule: 18] that are manufactured by SARTOMER; “ESDRIMER HU-22” manufactured by NIPPON STEEL & SUMIKIN CHEMICAL Co., Ltd.; “A-HBR-5” manufactured by Shin Nakamura Chemical Co., Ltd.; “NEW FRONTIER R-1150” manufactured by DAI-ICHI KOGYO SEIYAKU Co., Ltd.; and “HYPERTECH UR-101” manufactured by Nissan Chemical Corporation.

The weight average molecular weights (Mw) of the dendrimer-type (meth)acrylate resins and modified products (R9) thereof are preferably in the range of 1,000 to 30,000. The average number of (meth)acryloyl groups per molecule is preferably in the range of 5 to 30.

When the calixarene compound in the present embodiment is used as a photocurable resin material, it is preferable to add a photopolymerization initiator to the calixarene compound used. A suitable photopolymerization initiator may be selected according to the type of active energy rays used for irradiation etc. Specific examples of the photopolymerization initiator include: alkylphenone-based photopolymerization initiators such as 1-hydroxy-cyclohexyl-phenyl-ketone, 2-benzyl-2-dimethylamino-1-(4-morpholinophenyl)-butanone-1 and 2-(dimethylamino)-2-[(4-methylphenyl)methyl]-1-[4-(4-morpholinyl)phenyl]-1-butanone; acylphosphine oxide-based photopolymerization initiators such as 2,4,6-trimethylbenzoyl-diphenyl-phosphine oxide; and intramolecular hydrogen abstraction-type photopolymerization initiators such as benzophenone compounds. One of them may be used alone, or two or more may be used in combination.

Examples of commercial products of the photopolymerization initiator include “IRGACURE 127,” “IRGACURE 184,” “IRGACURE 250,” “IRGACURE 270,” “IRGACURE 290,” “IRGACURE 369E,” “IRGACURE 379EG,” “IRGACURE 500,” “IRGACURE 651,” “IRGACURE 754,” “IRGACURE 819,” “IRGACURE 907,” “IRGACURE 1173,” “IRGACURE 2959,” “IRGACURE MBF,” “IRGACURE TPO,” “IRGACURE OXE 01,” and “IRGACURE OXE 02” that are manufactured by BASF.

The amount of the photopolymerization initiator used is preferably in the range of 0.05 to 20 parts by mass and more preferably in the range of 0.1 to 10 parts by mass based on 100 parts by mass of the curable composition excluding an organic solvent.

The curable composition may be diluted with an organic solvent. Examples of the organic solvent include: alkylene glycol monoalkyl ethers such as ethylene glycol monomethyl ether, ethylene glycol monoethyl ether, ethylene glycol monopropyl ether, ethylene glycol monobutyl ether, and propylene glycol monomethyl ether; dialkylene glycol dialkyl ethers such as diethylene glycol dimethyl ether, diethylene glycol diethyl ether, diethylene glycol dipropyl ether, and diethylene glycol dibutyl ether; alkylene glycol alkyl ether acetates such as ethylene glycol monomethyl ether acetate, ethylene glycol monoethyl ether acetate, and propylene glycol monomethyl ether acetate; ketone compounds such as acetone, methyl ethyl ketone, cyclohexanone, and methyl amyl ketone; cyclic ethers such as dioxane; and ester compounds such as methyl 2-hydroxypropionate, ethyl 2-hydroxypropionate, ethyl 2-hydroxy-2-methylpropionate, ethyl ethoxyacetate, ethyl oxyacetate, methyl 2-hydroxy-3-methylbutanoate, 3-methoxybutyl acetate, 3-methyl-3-methoxybutyl acetate, ethyl formate, ethyl acetate, butyl acetate, methyl acetoacetate, and ethyl acetoacetate. One of them may be used alone, or two or more may be used in combination. The amount of the organic solvent added is appropriately controlled according to the desired viscosity of the composition.

The curable composition in the present embodiment may contain various additives according to its desired performance. Examples of the additives include an ultraviolet absorber, an antioxidant, a photosensitizer, a silicone-based additive, a silane coupling agent, a fluorine-based additive, a rheology controlling agent, a defoaming agent, an antistatic agent, an antifogging agent, an adhesion aid, an organic pigment, an inorganic pigment, an extender, an organic filler, and an inorganic filler.

The preferred embodiments of the present invention have been described. However, the present invention is not limited to the above-described embodiments.

### EXAMPLES

The present invention will be described more specifically by way of Production Examples and Examples. However, the present invention is not limited to these Examples. In the Examples, parts and % are based on mass unless otherwise specified.

The structure of a product (calixarene compound) was identified using ¹H-NMR, ¹³C-NMR, and FD-MS measurement performed under the following conditions.

The ¹H-NMR measurement was performed using “JNM-ECM400S” manufactured by JEOL RESONANCE Inc. under the following conditions.

Magnetic field intensity: 400 MHz

Number of scans: 16

Solvent: deuterated chloroform

Sample concentration: 2 mg/0.5 mL

The ¹³C-NMR measurement was performed using “JNM-ECM400S” manufactured by JEOL RESONANCE Inc. under the following conditions.

Magnetic field intensity: 100 MHz

Number of scans: 1000

Solvent: deuterated chloroform

Sample concentration: 2 mg/0.5 mL

The FD-MS measurement was performed using “JMS-T100GC AccuTOF” manufactured by JEOL Ltd. under the following conditions.

Measurement range: m/z=50.00 to 2000.00

Rate of change: 25.6 mA/min

Final current value: 40 mA

Cathode voltage: −10 kV

In the following description, Examples etc. in which the functional group (I) is a cyano group are classified into Example group <I>, and Examples etc. in which the functional group (I) is a maleate group are classified into Example group <II>. Examples etc. in which the functional group (I) is an acetylacetonate group are classified into Example group <III>, and Examples etc. in which the functional group (I) is an oxalate group are classified into Example group <IV>. Examples etc. in which the functional group (I) is a malonate group are classified into Example group <V>.

### Example Group <I>

### Synthesis Example 1

A 20 L separable four-necked flask equipped with a stirrer, a thermometer, and a reflux condenser tube was quickly charged with 1000 g (1.54 mol) of t-butyl-calix[4]arene, 1159 g (12.32 mol) of phenol, and 9375 mL of dehydrated toluene, and the mixture was stirred at 300 rpm under nitrogen flow. The t-butyl-calix[4]arene used as a raw material was not dissolved but was suspended. Then, while the flask was cooled in an ice bath, 1643 g (12.32 mol) of anhydrous aluminum chloride (III) was added in several portions. The solution turned into a light orange transparent solution, and the anhydrous aluminum chloride (III) precipitated on the bottom. The mixture was allowed to react at room temperature for 5 hours. Then the contents were transferred to a 1 L beaker, and 20 kg of ice, 10 L of 1N hydrochloric acid, and 20 L of chloroform were added to quench the reaction. The mixture turned into a light yellow transparent solution. This reaction mixture was transferred to a separatory funnel, and the organic layer was separated. Then the aqueous layer was extracted with 5 L of chloroform three times, and the extract was combined with the organic layer. The organic layer was pre-dried over anhydrous magnesium sulfate and filtered. The solvent was removed by evaporation using an evaporator, and a mixture of white crystals and a colorless transparent solution was thereby obtained. Methanol was added slowly to the mixture under stirring to reprecipitate the crystals. The white crystals were filtered on a Kiriyama funnel and washed with methanol. The obtained white crystals were vacuum dried (at 50° C. for 6 hours or longer) to thereby obtain 597 g of the target intermediate (A). The yield was 91%.

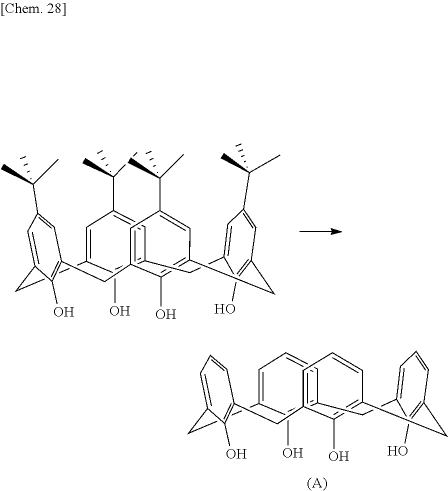

### Synthesis Example 2

A 2 L four-necked flask equipped with a stirrer, a thermometer, and a reflux condenser tube was charged with 205 g (1.52 mol) of n-hexanoyl chloride and 709 g (9.44 mol) of nitroethane, and the mixture was stirred. Then, while the flask was cooled in an ice bath, 243 g (1.82 mol) of anhydrous aluminum chloride (III) was added in several portions. The solution turned into a light orange transparent solution. The resulting solution was stirred at room temperature for 30 minutes, and 100 g (0.236 mol) of intermediate (α-1) was added in several portions. The mixture foamed, and the reaction proceeded to give an orange transparent solution. The solution was allowed to react at room temperature for 5 hours. Then the contents were slowly transferred to a 2 L beaker containing 450 mL of chloroform and 956 g of ice water to stop the reaction. Then 1N hydrochloric acid was added until the pH was 1. The reaction mixture was transferred to a separatory funnel, and the organic layer was separated. Next, the aqueous layer was extracted with 400 mL of chloroform three times, and the extract was combined with the organic layer. The organic layer was pre-dried over anhydrous magnesium sulfate and filtered. The solvent was removed by evaporation using an evaporator, and a yellow transparent solution was thereby obtained. While the solution was cooled in an ice bath, methanol was added to reprecipitate crystals. The white crystals were filtered on a Kiriyama funnel and recrystallized with chloroform and methanol. The obtained white crystals were vacuum dried (at 60° C. for 6 hours or longer) to thereby obtain 122 g of a compound represented by the following structural formula. The yield was 63%.

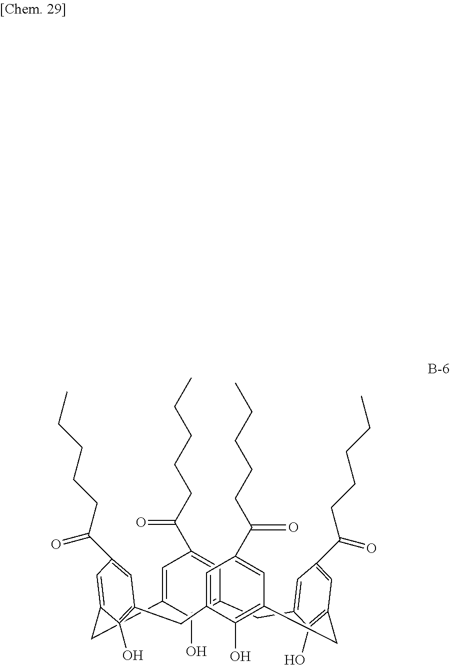 [SMILES: CCCCCC(=O)c1cc2c(O)c(c1)Cc1cc(C(=O)CCCCC)cc(c1O)Cc1cc(C(=O)CCCCC)cc(c1O)Cc1cc(C(=O)CCCCC)cc(c1O)C2]

### Synthesis Example 3

The same procedure as in Synthesis Example 2 was repeated except that butyl chloride was used instead of n-hexanoyl chloride, to thereby obtain 106 g of compound B-4 represented by the following structural formula. The yield was 64%.

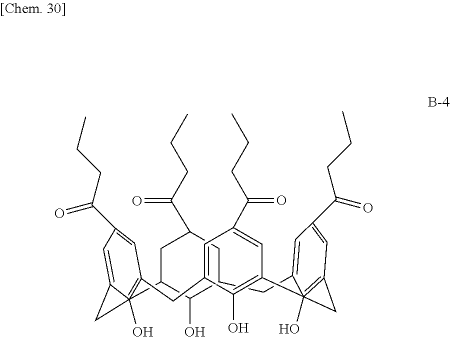

### Synthesis Example 4

The same procedure as in Synthesis Example 2 was repeated except that n-heptanoyl chloride was used instead of n-hexanoyl chloride, to thereby obtain 134 g of compound B-7 represented by the following structural formula. The yield was 65%.

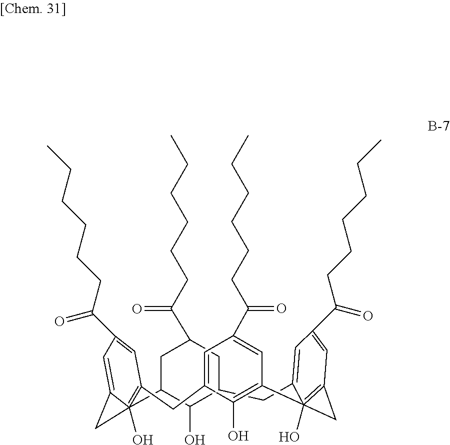

### Synthesis Example 5

The same procedure as in Synthesis Example 2 was repeated except that stearoyl chloride was used instead of n-hexanoyl chloride, to thereby obtain 228 g of compound B-18 represented by the following structural formula. The yield was 65%.

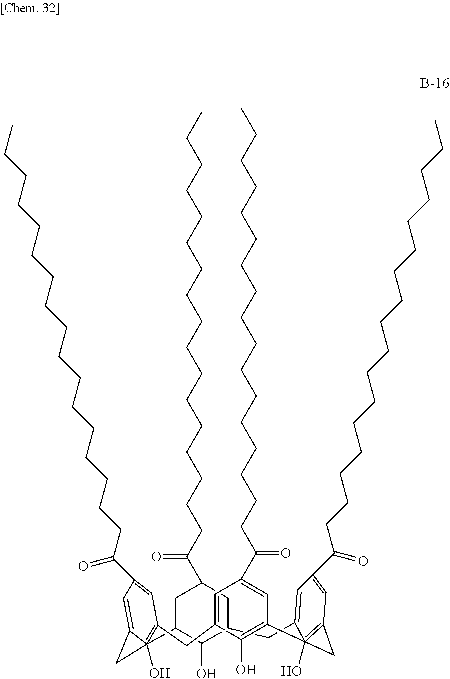

### Synthesis Example 6

A 500 mL four-necked flask equipped with a stirrer, a thermometer, and a reflux condenser tube was charged with 10.00 g (12.24 mmol) of B-6, 44.13 g (611.9 mmol) of tetrahydrofuran, 14.12 g (53.85 mmol) of triphenylphosphine, and 7.01 g (53.85 mmol) of hydroxyethyl methacrylate, and the mixture was stirred. The obtained ocher suspension solution was cooled with ice, and 12.10 g (53.85 mmol) of diisopropyl azodicarboxylate was added dropwise over 30 minutes. The reaction solution turned into an orange transparent solution, and the orange transparent solution was stirred at room temperature for 5 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:acetone=95:5) to thereby obtain a light yellow transparent liquid. The solvent was concentrated, and chloroform/methanol were added to reprecipitate crystals. The obtained white crystals were vacuum dried (at 60° C. for 6 hours or longer) to thereby obtain 2.65 g of the target compound C-6 with a yield of 23.3% and 4.98 g of the target compound D-6 with a yield of 39.1%.

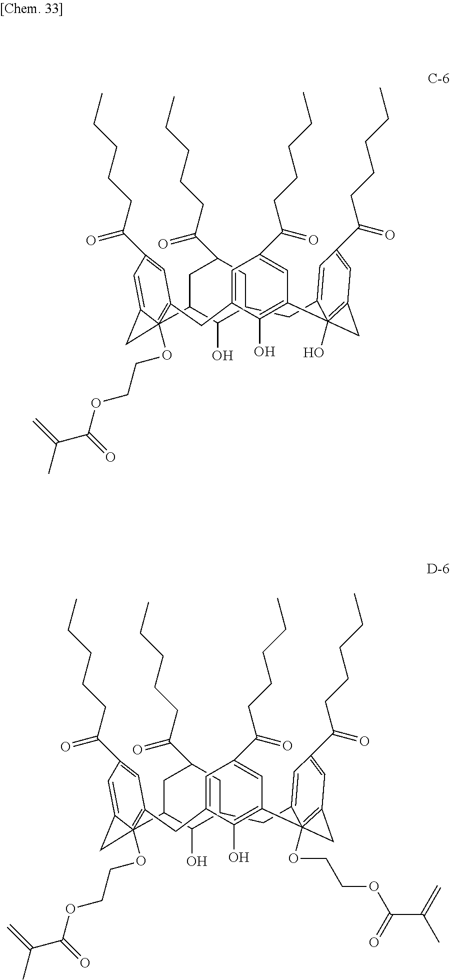

### Synthesis Example 7

The same procedure as in Synthesis Example 6 was repeated except that B-4 was used instead of B-6, to thereby obtain 1.89 g of the target compound C-4. The yield was 16.3%. 4.71 g of D-4 was obtained. The yield was 35.8%.

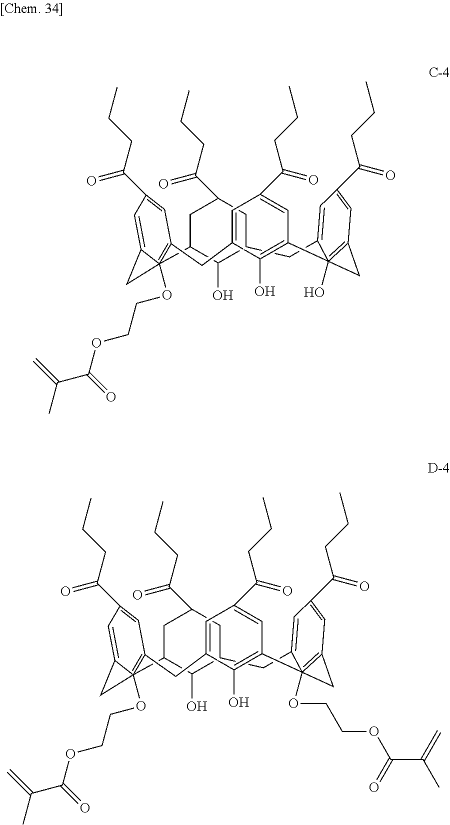

### Synthesis Example 8

The same procedure as in Synthesis Example 6 was repeated except that B-7 was used instead of B-6, to thereby obtain 2.32 g of the target compound C-7. The yield was 20.6%. 4.12 g of D-7 was obtained. The yield was 32.8%.

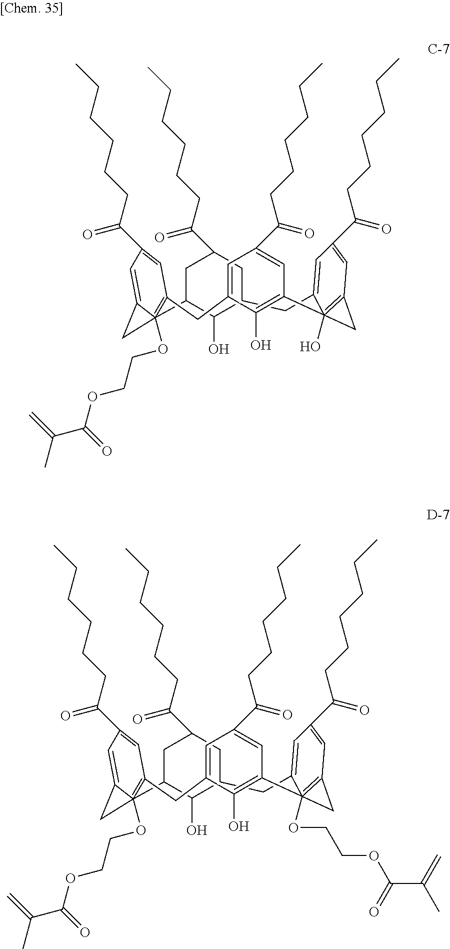

### Example 1

A 100 mL four-necked flask equipped with a stirrer, a thermometer, and a reflux condenser tube was charged with 1.00 g (1.076 mmol) of C-6, 15.73 g of anhydrous DMF, 0.155 g (3.874 mmol) of sodium hydride (60%, liquid paraffin dispersion), and 0.519 g (3.874 mmol) of 3-bromopropionitrile, and the mixture was stirred at room temperature for 16 hours. Ion exchanged water was added to stop the reaction, and 30 g of chloroform was added to extract the product. The mixture was washed twice with ion exchanged water, and the organic layer was pre-dried over anhydrous magnesium sulfate. The solvent was removed by evaporation using an evaporator under reduced pressure, and the obtained orange viscus liquid was purified by column chromatography (eluent: n-hexane:ethyl acetate=85:15) to thereby obtain 0.482 g of the target compound 1-6. The yield was 41.1%.

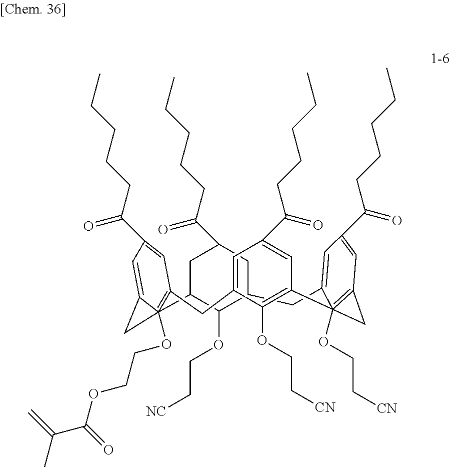

### Example 2

The same procedure as in Example 1 was repeated except that c-4 was used instead of C-6, to thereby obtain 0.369 g of the target compound 1-4. The yield was 30.9%.

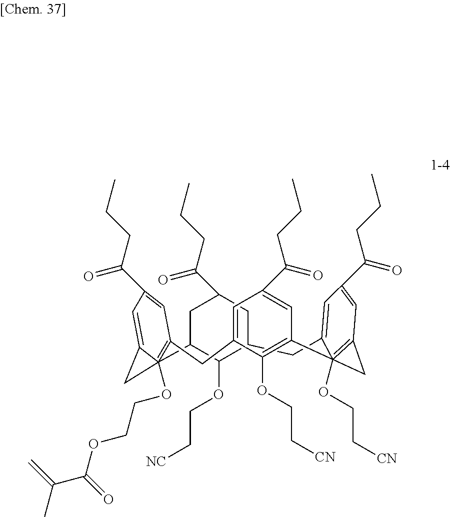

### Example 3

The same procedure as in Example 1 was repeated except that C-7 was used instead of C-6, to thereby obtain 0.684 g of the target compound 1-7. The yield was 58.9%.

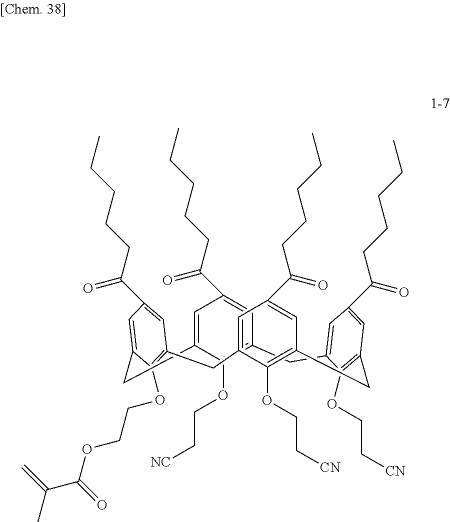

### Example 4

The same procedure as in Example 1 was repeated except that 4-bromobutyronitrile was used instead of 3-bromopropionitrile, to thereby obtain 0.539 g of the target compound 2-6. The yield was 44.3%.

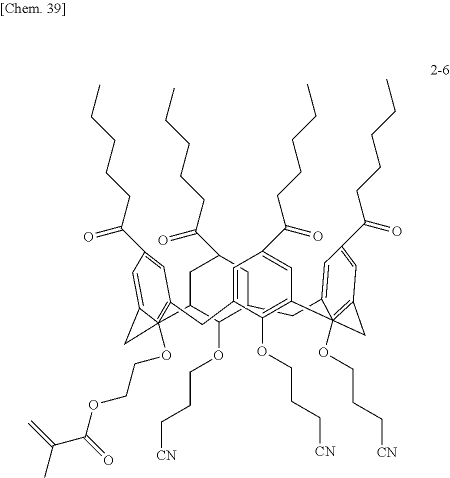

### Example 5

The same procedure as in Example 4 was repeated except that C-4 was used instead of C-6, to thereby obtain 0.476 g of the target compound 2-4. The yield was 38.2%.

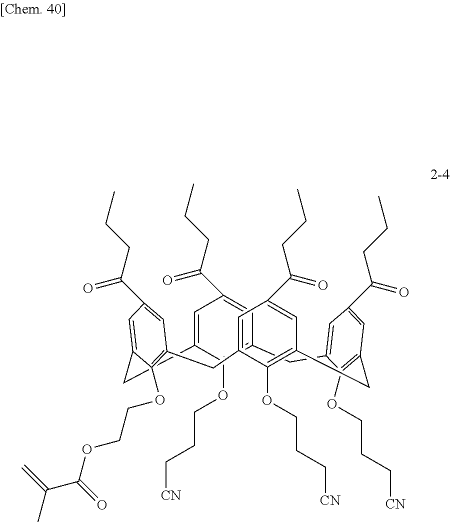

### Example 6

The same procedure as in Example 4 was repeated except that C-7 was used instead of C-6, to thereby obtain 0.567 g of the target compound 2-7. The yield was 47.1%.

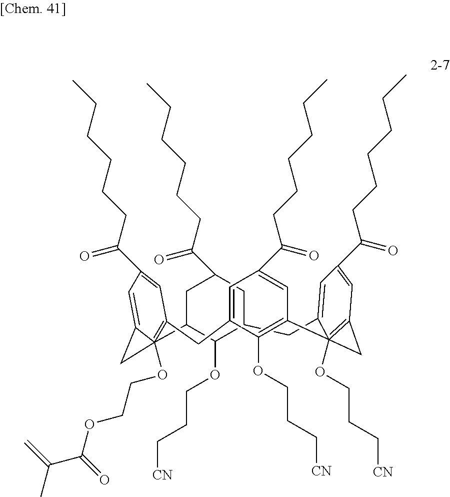

The same procedure as in Example 1 was repeated except that D-6 was used instead of C-6, to thereby obtain 0.524 g of the target compound 3-6. The yield was 47.6%.

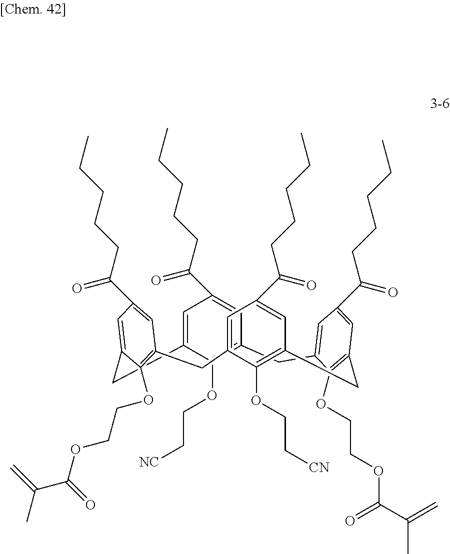

### Example 8

The same procedure as in Example 7 was repeated except that 4-bromobutyronitrile was used instead of 3-bromopropionitrile, to thereby obtain 0.518 g of the target compound 4-6. The yield was 47.0%.

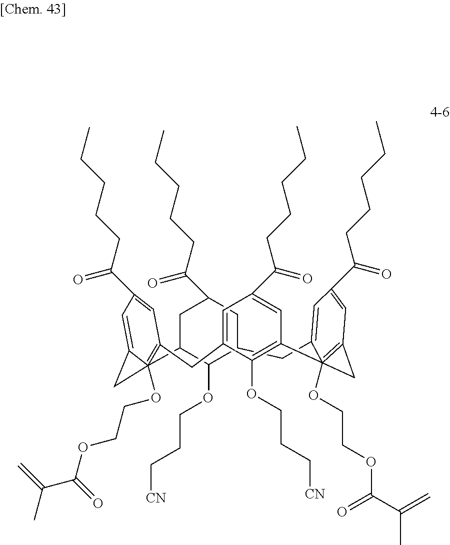

### Synthesis Example 9

The same procedure as in Synthesis Example 6 was repeated except that hydroxyethyl acrylate was used instead of hydroxyethyl methacrylate, to thereby obtain 2.91 g of the target compound E-6. The yield was 26.0%. 4.83 g of F-6 was obtained. The yield was 39.0%.

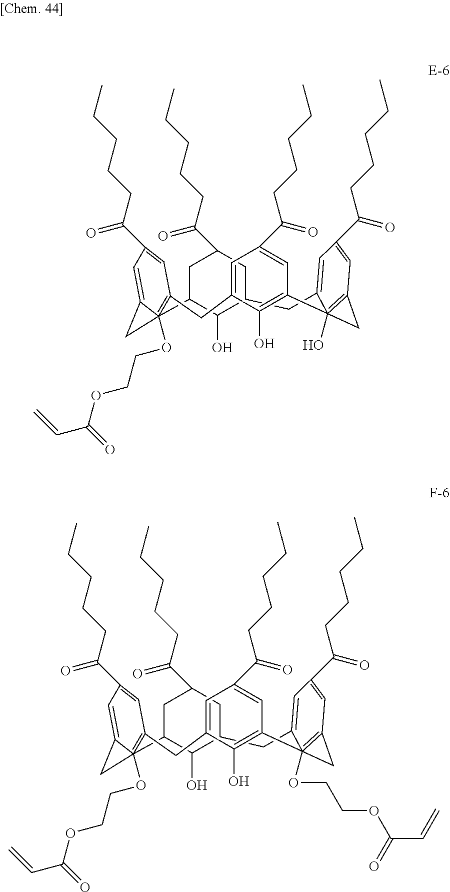

### Example 9

The same procedure as in Synthesis Example 9 was repeated except that E-6 was used instead of C-6, to thereby obtain 0.461 g of the target compound 5-6. The yield was 39.3%.

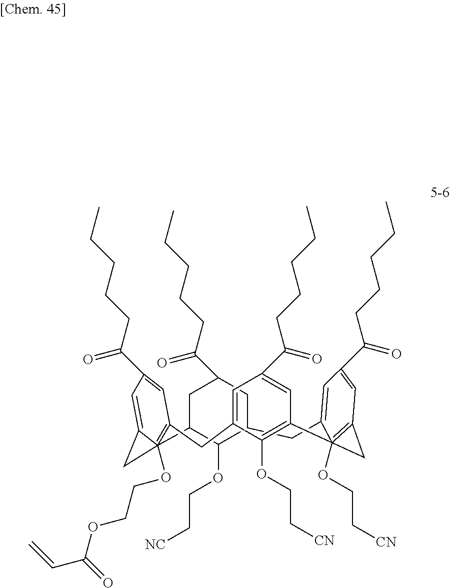

### Example 10

The same procedure as in Example 9 was repeated except that 4-bromobutyronitrile was used instead of 3-bromopropionitrile, to thereby obtain 0.399 g of the target compound 6-6. The yield was 34.0%.

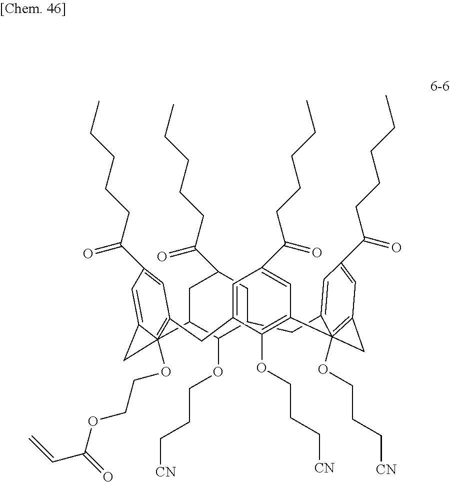

### Example 11

The same procedure as in Example 1 was repeated except that E-6 was used instead of C-6, to thereby obtain 0.483 g of the target compound 7-6. The yield was 43.8%.

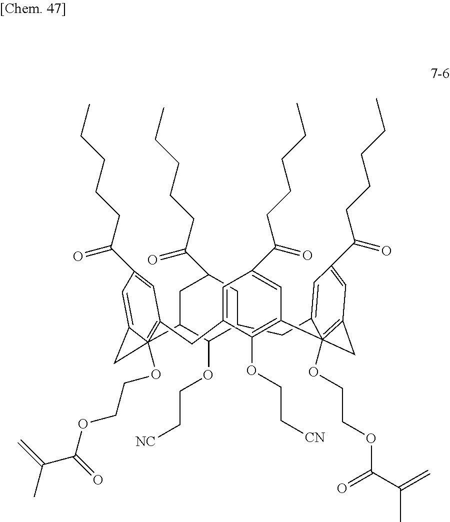

### Example 12

The same procedure as in Example 11 was repeated except that 4-bromobutyronitrile was used instead of 3-bromopropionitrile, to thereby obtain 0.367 g of the target compound 8-6. The yield was 33.3%.

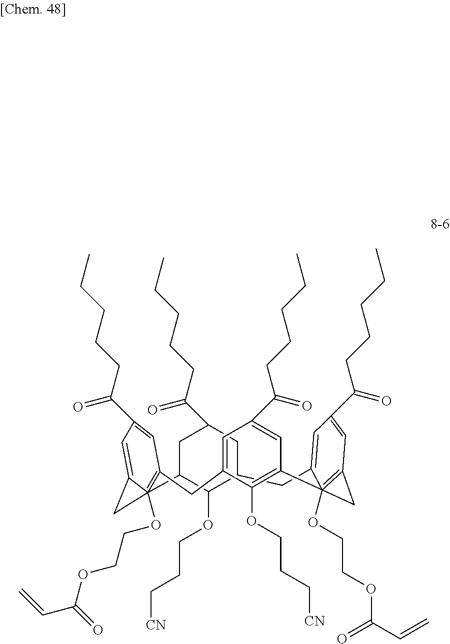

### Synthesis Example 10

The same procedure as in Synthesis Example 6 was repeated except that hydroxypropyl methacrylate was used instead of hydroxyethyl methacrylate, to thereby obtain 2.67 g of the target compound G-6. The yield was 23.1%. 4.44 g of H-6 was obtained. The yield was 33.9%.

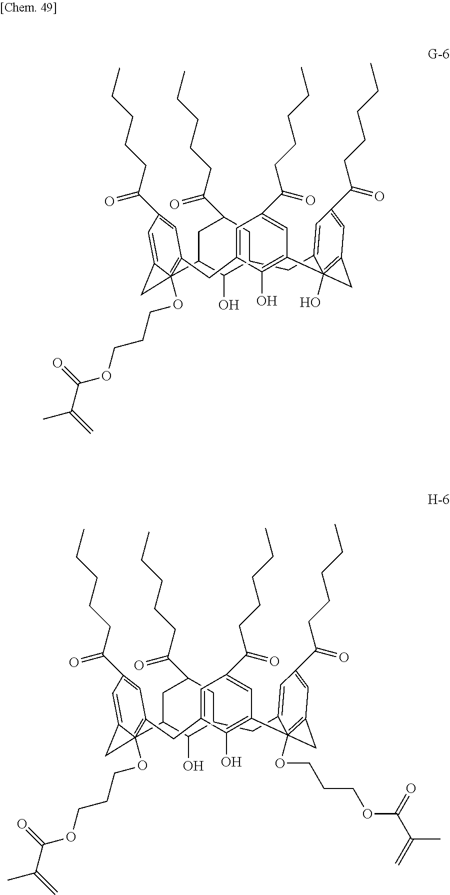

### Example 13

The same procedure as in Example 1 was repeated except that G-6 was used instead of C-6, to thereby obtain 0.312 g of the target compound 9-6. The yield was 26.7%.

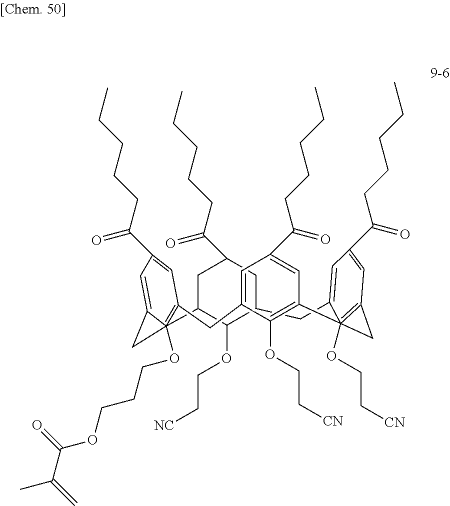

### Example 14

The same procedure as in Example 13 was repeated except that 4-bromobutyronitrile was used instead of 3-bromopropionitrile, to thereby obtain 0.313 g of the target compound 10-6. The yield was 26.8%.

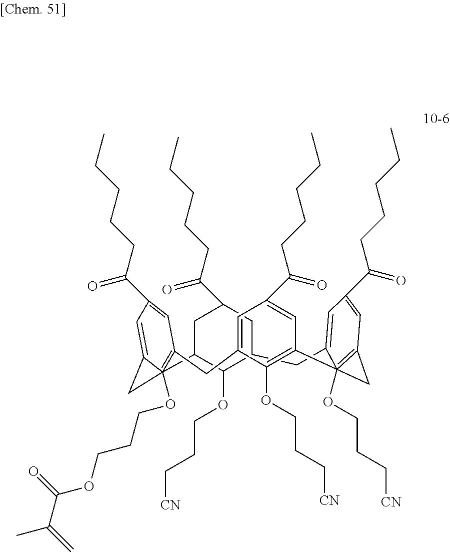

### Example 15

The same procedure as in Example 1 was repeated except that H-6 was used instead of C-6, to thereby obtain 0.387 g of the target compound 11-6. The yield was 35.2%.

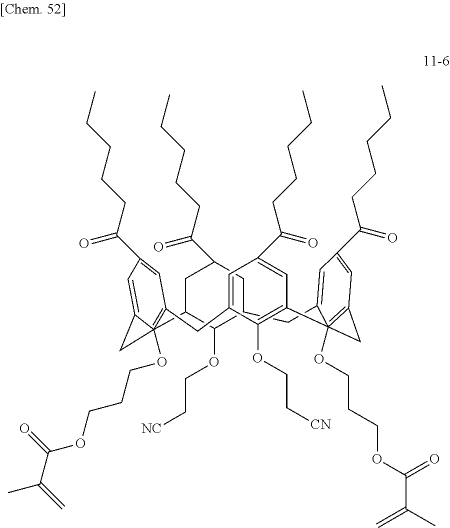

The same procedure as in Synthesis Example 25 was repeated except that 4-bromobutyronitrile was used instead of 3-bromopropionitrile, to thereby obtain 0.369 g of the target compound 12-6. The yield was 33.6%.

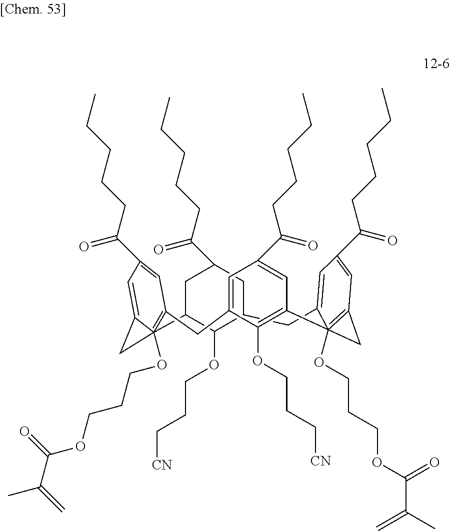

### Synthesis Example 10

The same procedure as in Synthesis Example 6 was repeated except that 4-hydroxybutyl methacrylate was used instead of hydroxyethyl methacrylate, to thereby obtain 2.23 g of the target compound I-6. The yield was 19.3%. 6.11 g of J-6 was obtained. The yield was 46.7%.

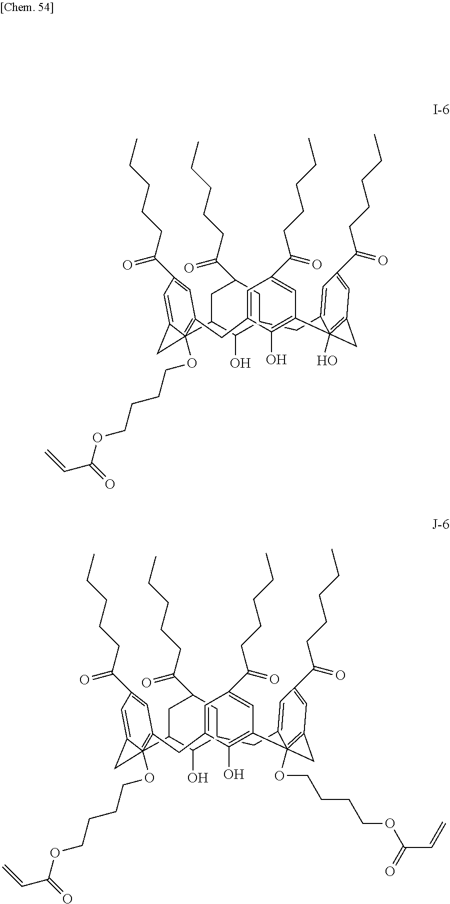

### Example 17

The same procedure as in Example 1 was repeated except that I-6 was used instead of C-6, to thereby obtain 0.339 g of the target compound 13-6. The yield was 29.0%.

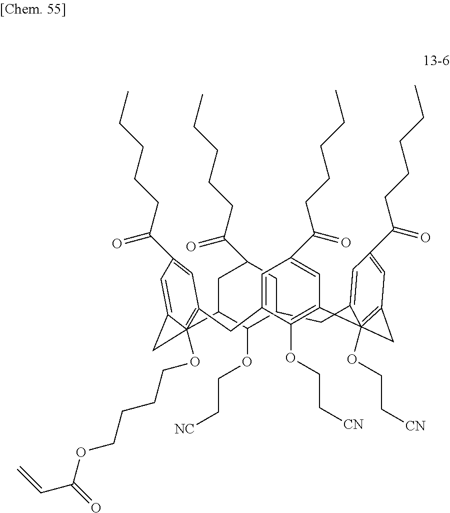

### Example 18

The same procedure as in Example 17 was repeated except that 4-bromobutyronitrile was used instead of 3-bromopropionitrile, to thereby obtain 0.376 g of the target compound 14-6. The yield was 32.2%.

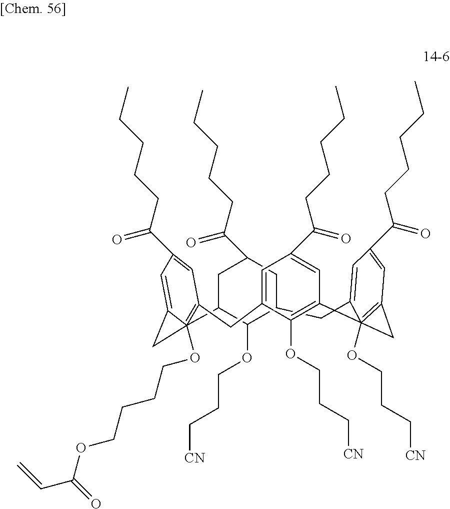

### Example 19

The same procedure as in Example 1 was repeated except that J-6 was used instead of C-6, to thereby obtain 0.342 g of the target compound 15-6. The yield was 31.1%.

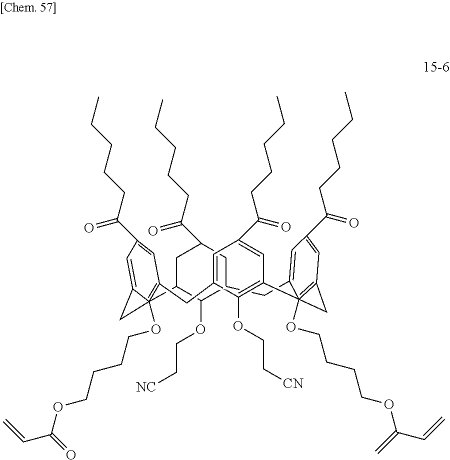

### Example 20

The same procedure as in Example 19 was repeated except that 4-bromobutyronitrile was used instead of 3-bromopropionitrile, to thereby obtain 0.281 g of the target compound 12-6. The yield was 25.6%.

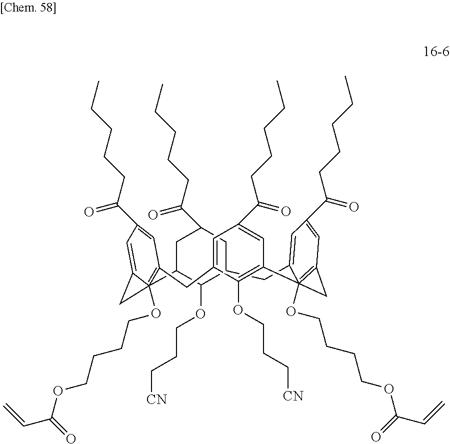

### Synthesis Example 11

A 500 mL four-necked flask equipped with a stirrer, a thermometer, and a reflux condenser tube was charged with 92.6 g (113.33 mmol) of B-6 and 944.52 g of diethylene glycol monomethyl ether, and the mixture was stirred. Next, 46.4 mL (906.64 mmol) of hydrazine monohydrate was added to the resulting white suspension solution, and 50.9 g (906.64 mmol) of potassium hydroxide pellets were added. The mixture was stirred at 100° C. for 30 minutes and heat-refluxed for 8 hours. A yellow transparent solution. After the reaction, the resulting solution was cooled to 90° C. 92.6 mL of ion exchanged water was added, and the mixture was stirred for 30 minutes. The mixture was cooled to room temperature, and 6N hydrochloric acid was added until the pH was 1. Then 300 g of chloroform was added, and the organic layer was separated. Next, the aqueous layer was extracted with 300 g of chloroform three times, and the extract was combined with the organic layer. The organic layer was pre-dried over anhydrous magnesium sulfate and filtered. The solvent was removed by evaporation using an evaporator, and an orange viscus liquid was thereby obtained. Methanol was added to reprecipitate crystals. The white crystals were filtered on a Kiriyama funnel, and the obtained milky white crystals were vacuum dried (at 60° C. for 6 hours or longer) to thereby obtain 54.34 g of the target compound K-6. The yield was 63.0%.

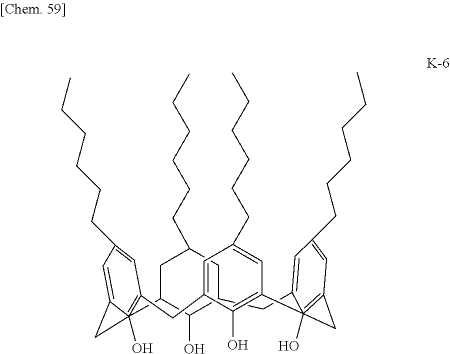

### Synthesis Example 12

The same procedure as in Synthesis Example 11 was repeated except that B-4 was used instead of B-6, to thereby obtain 72.45 g of the target compound K-4. The yield was 833.1%.

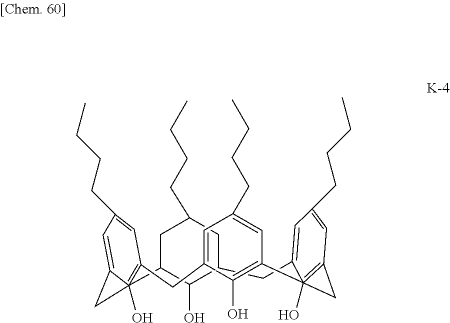

### Synthesis Example 13

The same procedure as in Synthesis Example 11 was repeated except that B-7 was used instead of B-6, to thereby obtain 78.4 g of the target compound K-7. The yield was 82.7%.

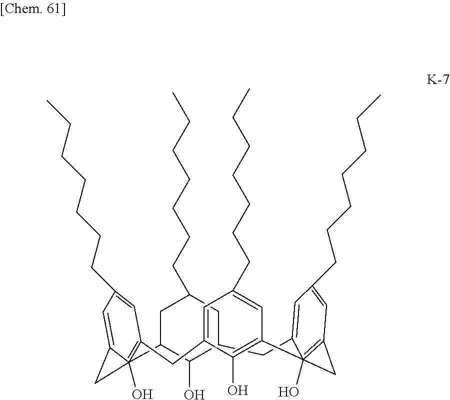

### Synthesis Example 14

The same procedure as in Synthesis Example 11 was repeated except that B-18 was used instead of B-6, to thereby obtain 37.9 g of the target compound K-18. The yield was 96.0%.

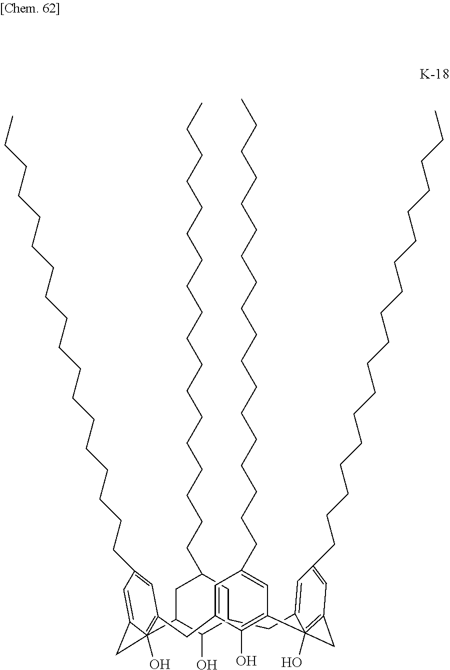

### Synthesis Example 15

Referring to known documents (Tetrahedron Letters, 43(43), 7691-7693; 2002, and Tetrahedron Letters, 48(5), 905-12; 1992), K-1 was synthesized according to the following scheme (yield amount 75 g, yield 66.6%).

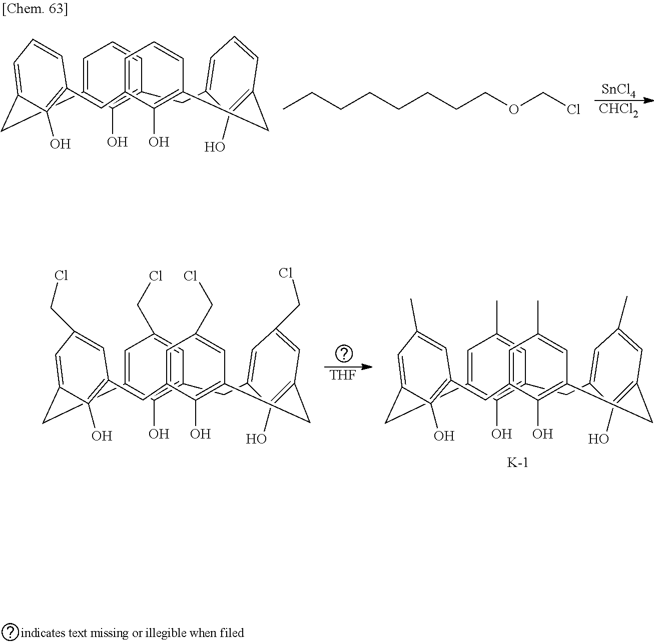

### Synthesis Example 16

The same procedure as in Synthesis Example 6 was repeated except that K-6 was used instead of B-6, to thereby obtain 2.65 g of the target compound L-6. The yield was 23.1%. 6.11 g of M-6 was obtained. The yield was 47.2%.

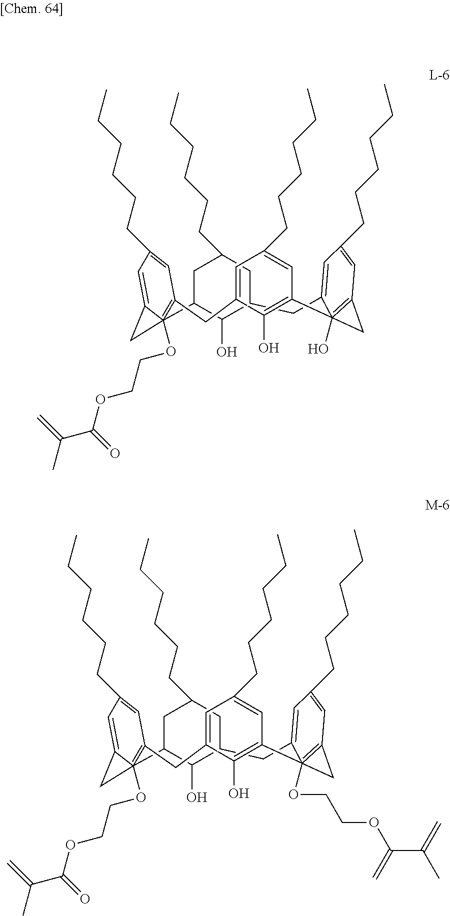

### Synthesis Example 17

The same procedure as in Synthesis Example 16 was repeated except that K-4 was used instead of K-6, to thereby obtain 2.19 g of the target compound L-4. The yield was 18.7%. 4.88 g of M-4 was obtained. The yield was 36.3%.

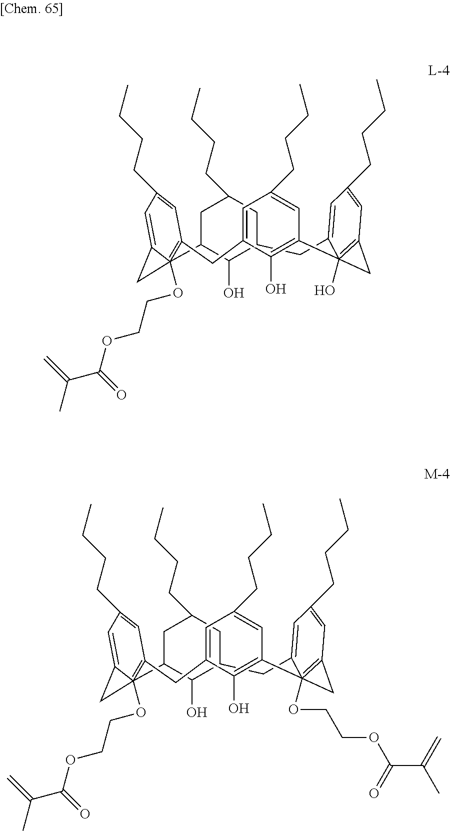

### Synthesis Example 18

The same procedure as in Synthesis Example 16 was repeated except that K-7 was used instead of K-6, to thereby obtain 2.32 g of the target compound L-7. The yield was 20.4%. 3.98 g of M-7 was obtained. The yield was 31.2%.

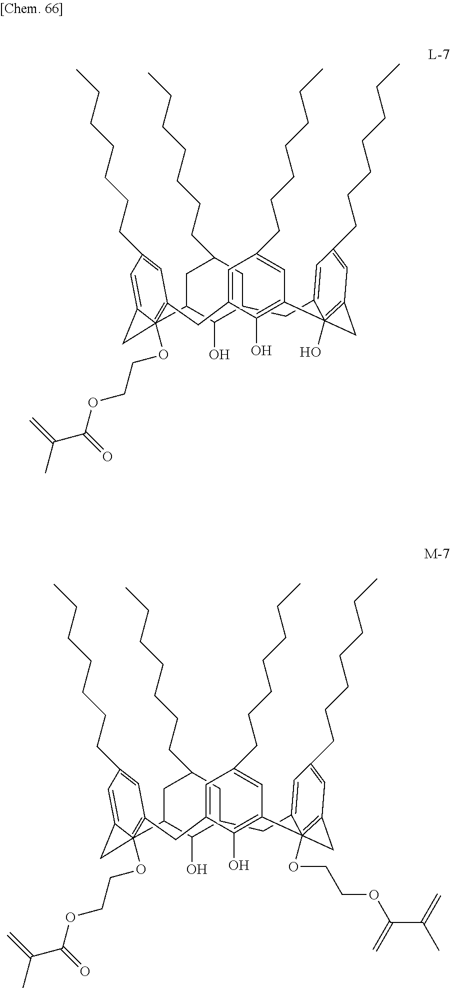

### Synthesis Example 19

The same procedure as in Synthesis Example 16 was repeated except that K-18 was used instead of K-6, to thereby obtain 2.29 g of the target compound L-18. The yield was 21.4%. 7.48 g of M-18 was obtained. The yield was 65.8%.

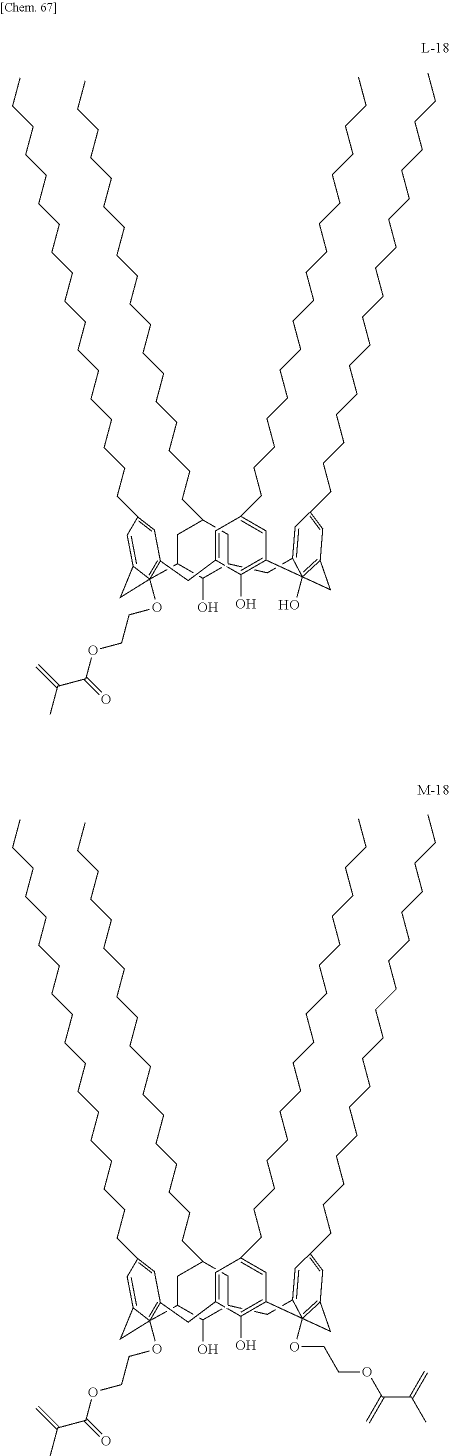

### Synthesis Example 20

The same procedure as in Synthesis Example 16 was repeated except that G-1 was used instead of G-6, to thereby obtain 1.34 g of the target compound L-1. The yield was 10.9%. 2.98 g of M-1 was obtained. The yield was 20.3%.

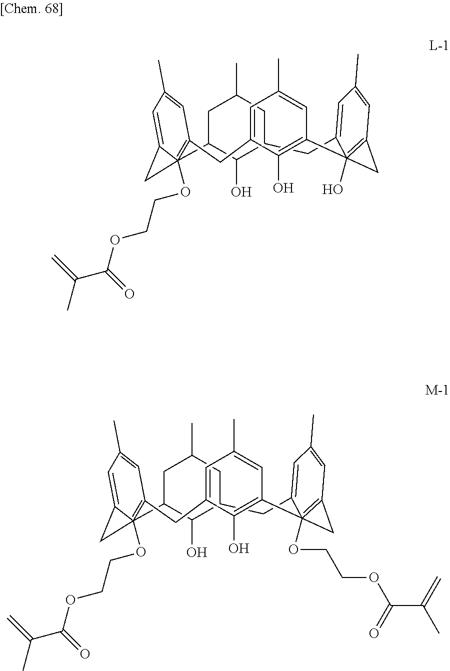

### Example 21

The same procedure as in Example 1 was repeated except that L-6 was used instead of C-6, to thereby obtain 0.567 g of the target compound 17-6. The yield was 48.0%.

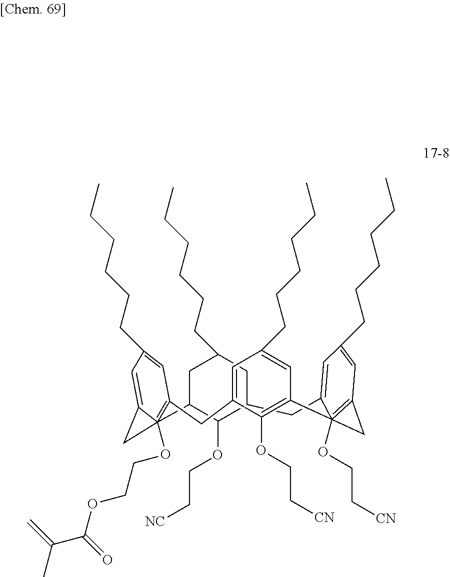

### Example 22

The same procedure as in Example 21 was repeated except that L-4 was used instead of L-6, to thereby obtain 0.498 g of the target compound 17-4. The yield was 41.2%.

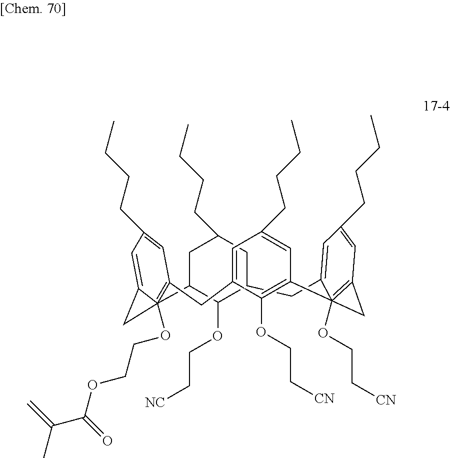

### Example 23

The same procedure as in Example 21 was repeated except that L-7 was used instead of L-6, to thereby obtain 0.500 g of the target compound 17-7. The yield was 42.7%.

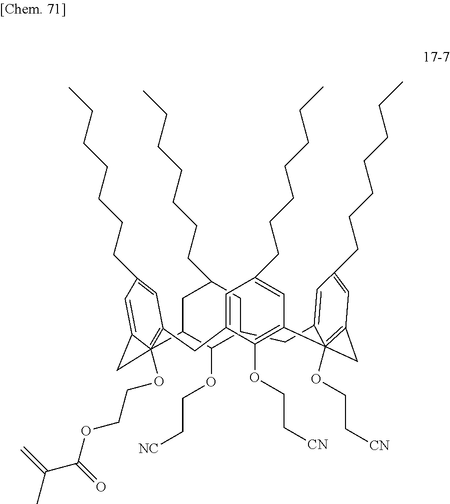

### Example 24

The same procedure as in Example 21 was repeated except that L-18 was used instead of L-6, to thereby obtain 0.621 g of the target compound 17-18. The yield was 56.3%.

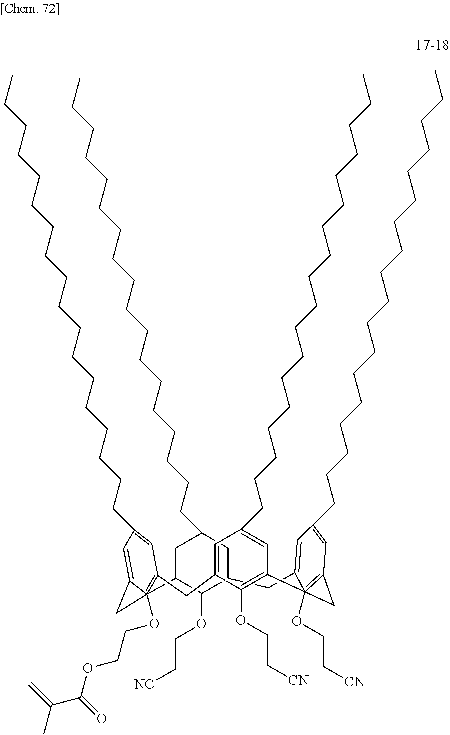

### Example 25

The same procedure as in Example 21 was repeated except that L-1 was used instead of L-6, to thereby obtain 0.329 g of the target compound 17-1. The yield was 25.9%.

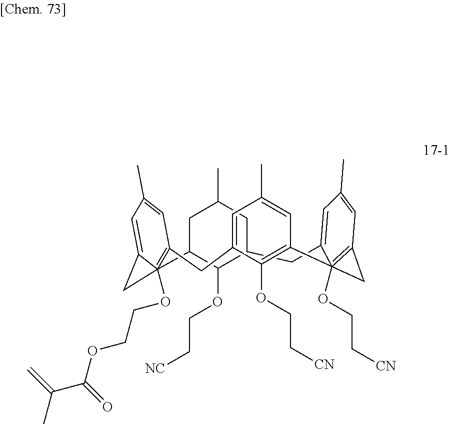

### Example 26

The same procedure as in Example 21 was repeated except that 4-bromobutyronitrile was used instead of 3-bromopropionitrile, to thereby obtain 0.529 g of the target compound 18-6. The yield was 43.0%.

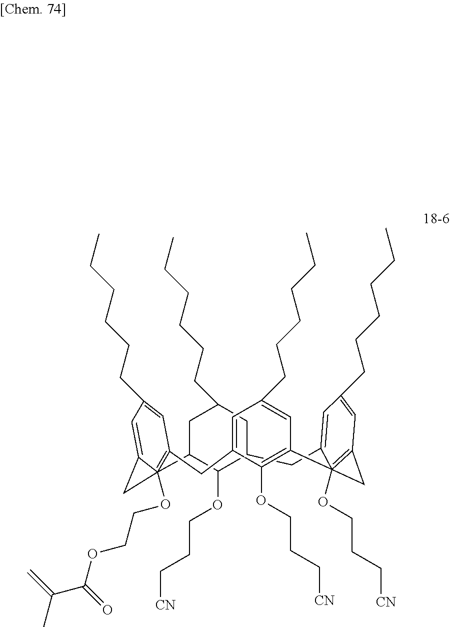

### Example 27

The same procedure as in Example 26 was repeated except that L-4 was used instead of L-6, to thereby obtain 0.551 g of the target compound 18-4. The yield was 43.6%.

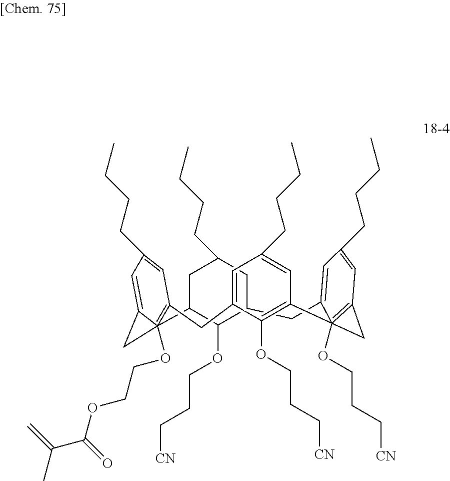

### Example 28

The same procedure as in Example 26 was repeated except that L-7 was used instead of L-6, to thereby obtain 0.572 g of the target compound 18-7. The yield was 47.0%.

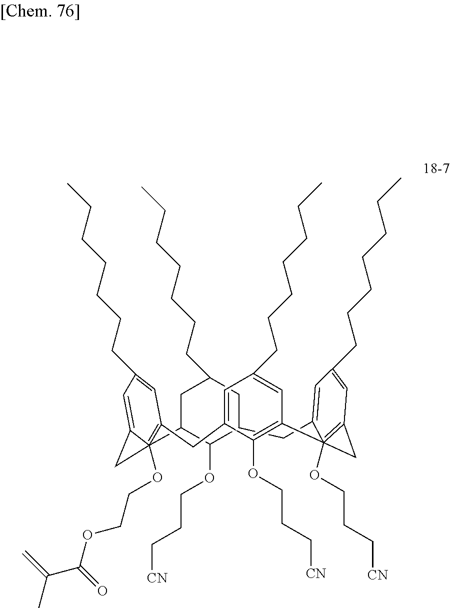

### Example 29

The same procedure as in Example 26 was repeated except that L-18 was used instead of L-6, to thereby obtain 0.711 g of the target compound 18-18. The yield was 62.9%.

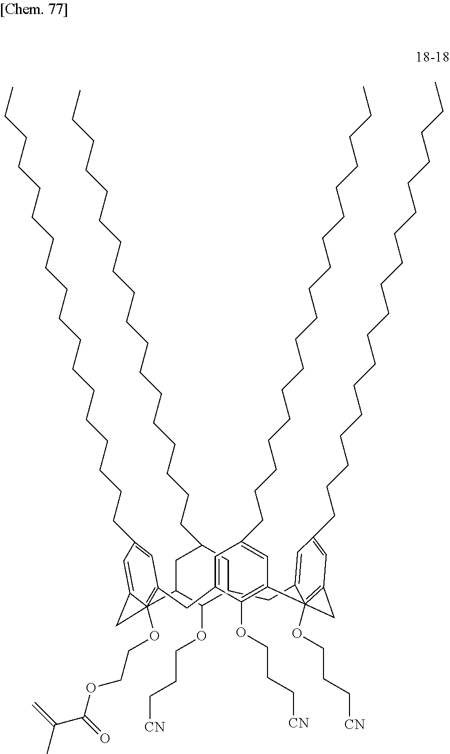

### Example 30

The same procedure as in Example 26 was repeated except that L-1 was used instead of L-6, to thereby obtain 0.343 g of the target compound 18-1. The yield was 25.6%.

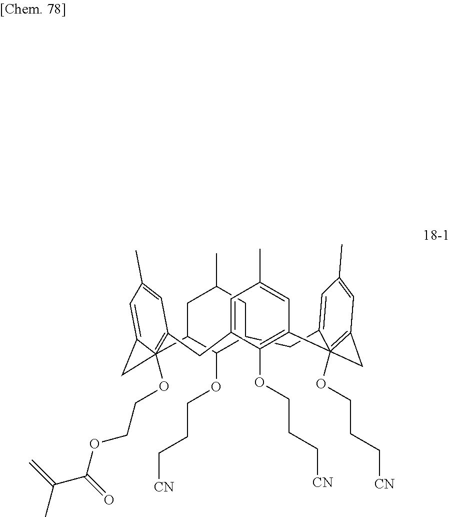

### Example 31

The same procedure as in Example 1 was repeated except that M-6 was used instead of L-6, to thereby obtain 0.609 g of the target compound 19-6. The yield was 55.0%.

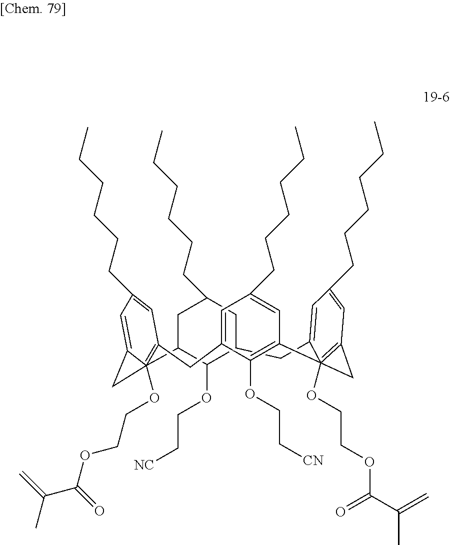

### Example 32

The same procedure as in Example 31 was repeated except that 4-bromobutyronitrile was used instead of 3-bromopropionitrile, to thereby obtain 0.587 g of the target compound 20-6. The yield was 51.7%.

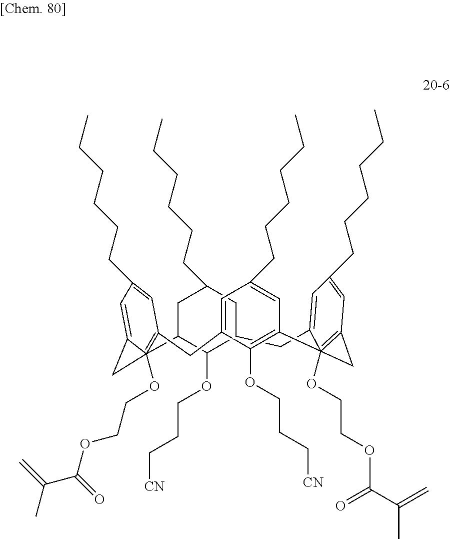

### Synthesis Example 21

The same procedure as in Synthesis Example 18 was repeated except that hydroxyethyl acrylate was used instead of hydroxyethyl methacrylate, to thereby obtain 2.89 g of the target compound N-6. The yield was 25.6%. 4.80 g of O-6 was obtained. The yield was 38.1%.

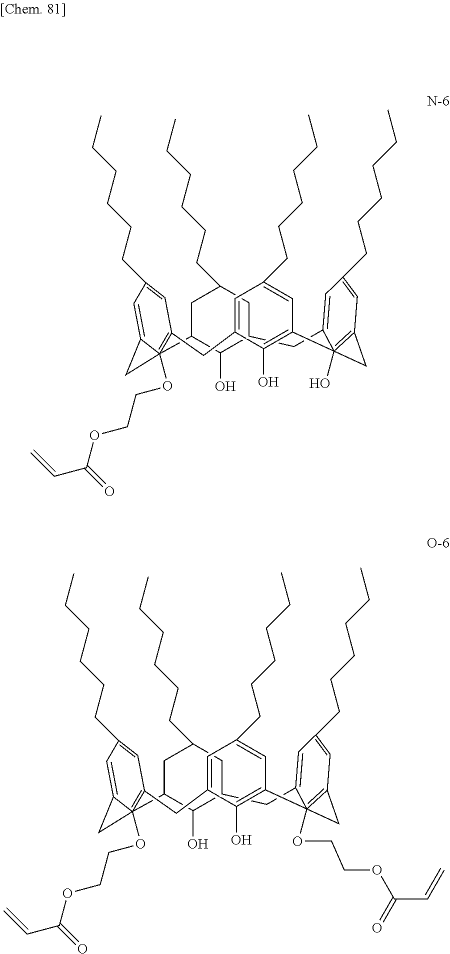

### Example 33

The same procedure as in Example 1 was repeated except that N-6 was used instead of C-6, to thereby obtain 0.0.519 g of the target compound 21-6. The yield was 43.8%.

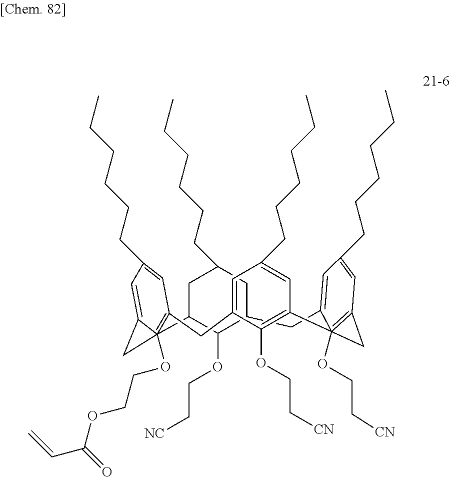

### Example 34

The same procedure as in Example 33 was repeated except that 4-bromobutyronitrile was used instead of 3-bromopropionitrile, to thereby obtain 0.507 g of the target compound 22-6. The yield was 41.1%.

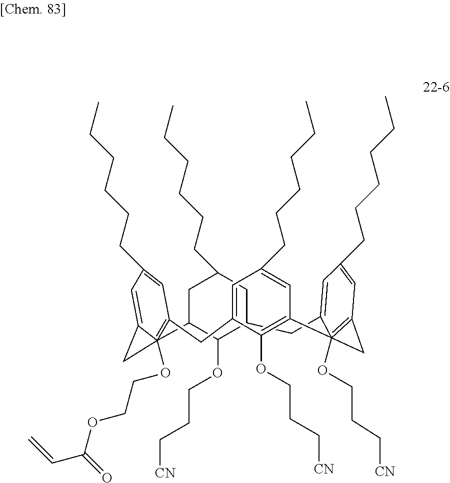

### Example 35

The same procedure as in Example 1 was repeated except that O-6 was used instead of C-6, to thereby obtain 0.635 g of the target compound 23-6. The yield was 57.3%.

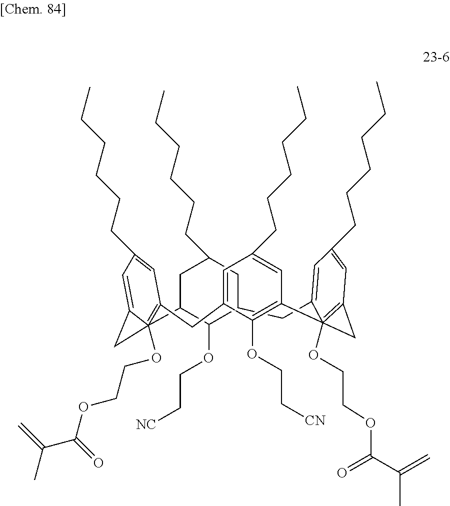

### Example 36

The same procedure as in Example 35 was repeated except that 4-bromobutyronitrile was used instead of 3-bromopropionitrile, to thereby obtain 0.599 g of the target compound 24-6. The yield was 52.5%.

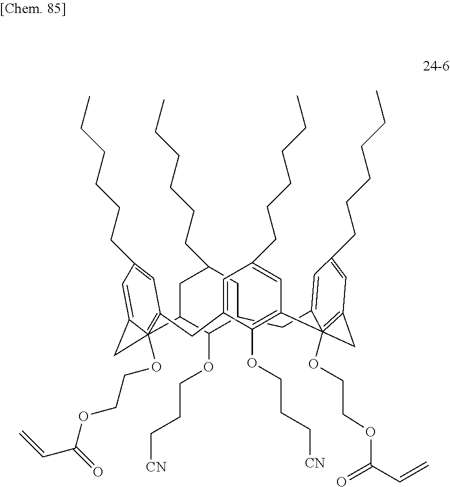

### Synthesis Example 22

The same procedure as in Example 16 was repeated except that hydroxypropyl methacrylate was used instead of hydroxyethyl methacrylate, to thereby obtain 2.33 g of the target compound P-6. The yield was 20.0%. 4.44 g of Q-6 was obtained. The yield was 33.3%.

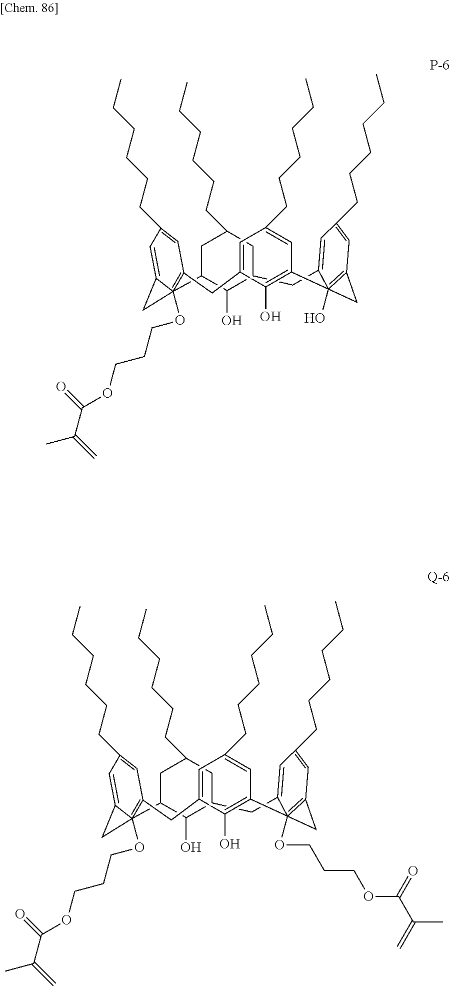

### Example 37

The same procedure as in Example 1 was repeated except that P-6 was used instead of C-6, to thereby obtain 0.0.484 g of the target compound 25-6. The yield was 41.0%.

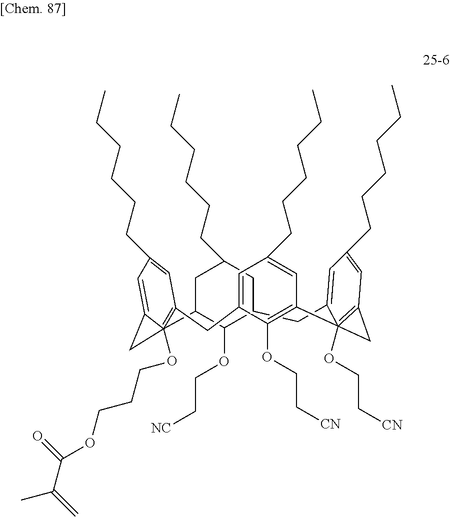

### Example 38

The same procedure as in Example 37 was repeated except that 4-bromobutyronitrile was used instead of 3-bromopropionitrile, to thereby obtain 0.556 g of the target compound 26-6. The yield was 45.3%.

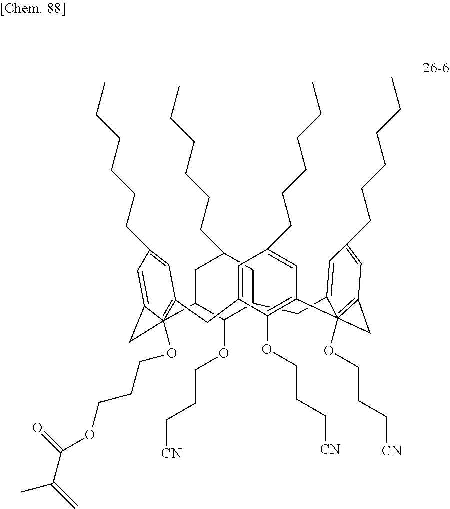

### Example 39

The same procedure as in Example 1 was repeated except that Q-6 was used instead of C-6, to thereby obtain 0.0.499 g of the target compound 27-6. The yield was 45.1%.

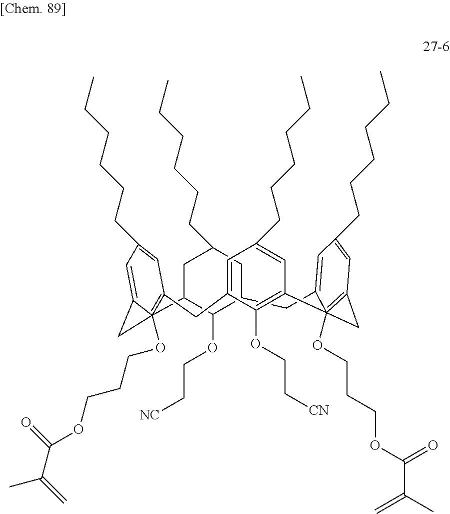

### Example 40

The same procedure as in Example 39 was repeated except that 4-bromobutyronitrile was used instead of 3-bromopropionitrile, to thereby obtain 0.482 g of the target compound 28-6. The yield was 42.6%.

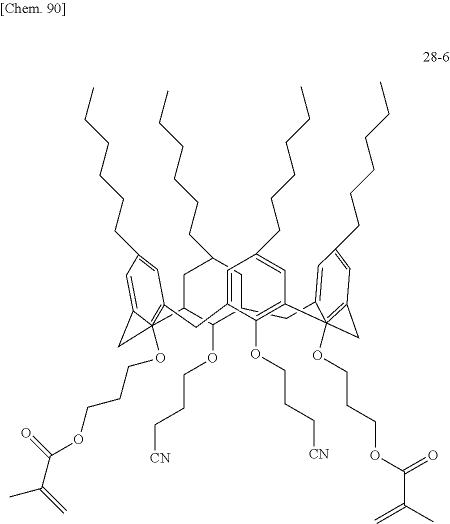

### Synthesis Example 23

The same procedure as in Synthesis Example 16 was repeated except that 4-hydroxybutyl acrylate was used instead of hydroxyethyl methacrylate, to thereby obtain 3.63 g of the target compound R-6. The yield was 31.1%. 5.48 g of S-6 was obtained. The yield was 41.1%.

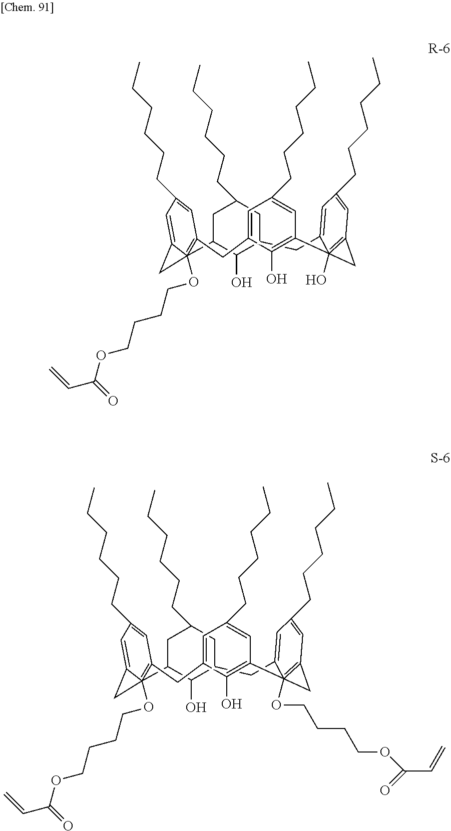

### Example 41

The same procedure as in Example 1 was repeated except that R-6 was used instead of C-6, to thereby obtain 0.513 g of the target compound 29-6. The yield was 43.5%.

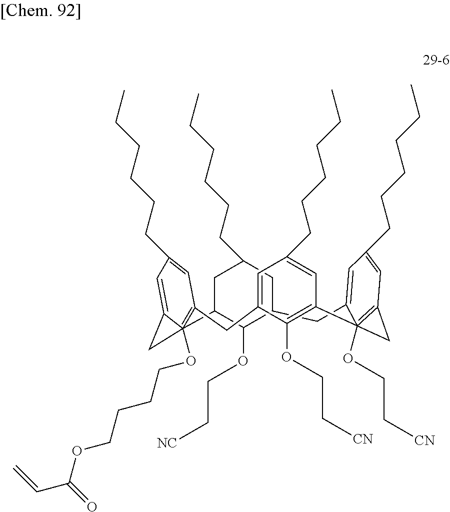

### Example 42

The same procedure as in Example 41 was repeated except that 4-bromobutyronitrile was used instead of 3-bromopropionitrile, to thereby obtain 0.497 g of the target compound 30-6. The yield was 40.5%.

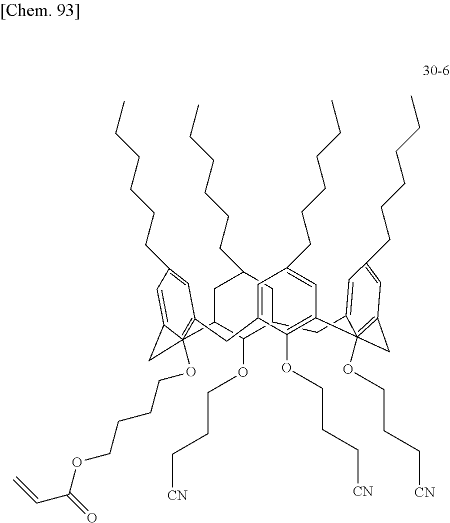

### Example 43

The same procedure as in Example 1 was repeated except that S-6 was used instead of C-6, to thereby obtain 0.527 g of the target compound 31-6. The yield was 47.7%.

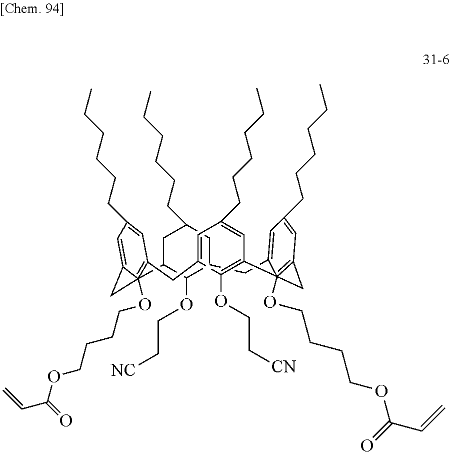

### Example 44

The same procedure as in Example 1 was repeated except that M-18 was used instead of C-6 and that valeronitrile was used instead of 3-bromopropionitrile, to thereby obtain 0.519 g of the target compound 32-18. The yield was 45.8%.

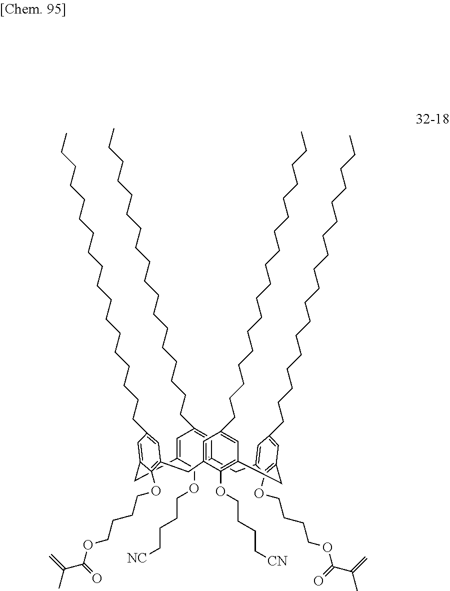

### Synthesis Example 24

A 1 L four-necked flask equipped with a stirrer, a thermometer, and a reflux condenser tube was charged with 20.00 g (26.276 mmol) of K-6, 400 g of anhydrous acetonitrile, 15.29 g (105.11 mmol) of potassium carbonate, 10.511 g (10.511 mmol) of potassium iodide, and 32.158 g (210.21 mmol) of methyl 2-bromoacetate, and the mixture was stirred at 70° C. for 6 hours. The mixture was cooled to room temperature, and ion exchanged water and 1N hydrochloric acid were added until the pH was 6. Then 500 g of chloroform was added. The reaction mixture was transferred to a separatory funnel, and the organic layer was separated. Then the aqueous layer was extracted with 100 g of chloroform three times, and the extract was combined with the organic layer. The organic layer was pre-dried over anhydrous magnesium sulfate and filtered. The solvent was removed by evaporation using an evaporator, and a red waxy solid was thereby obtained. The obtained red waxy solid was vacuum dried (at 60° C. for 6 hours or longer) to thereby obtain 21.67 g of the target compound T-6. The yield was 78.6%.

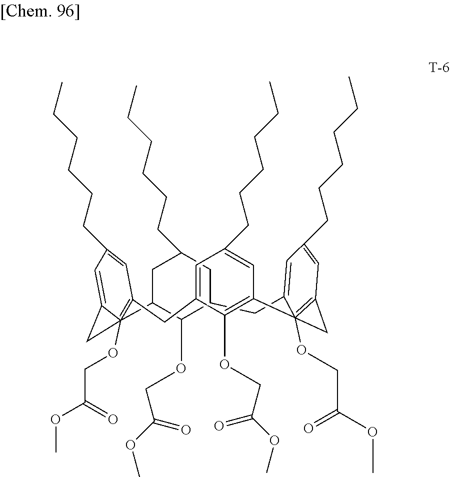

### Synthesis Example 25

The same procedure as in Synthesis Example 24 was repeated except that K-4 was used instead of K-6, to thereby obtain 21.81 g of the target compound T-4. The yield was 75.5%.

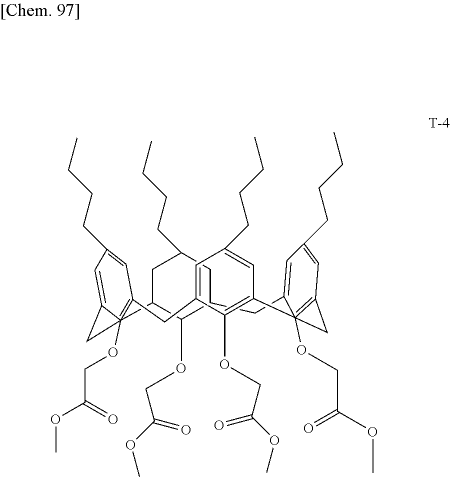

### Synthesis Example 26

The same procedure as in Synthesis Example 24 was repeated except that K-7 was used instead of K-6, to thereby obtain 20.98 g of the target compound T-7. The yield was 77.5%.

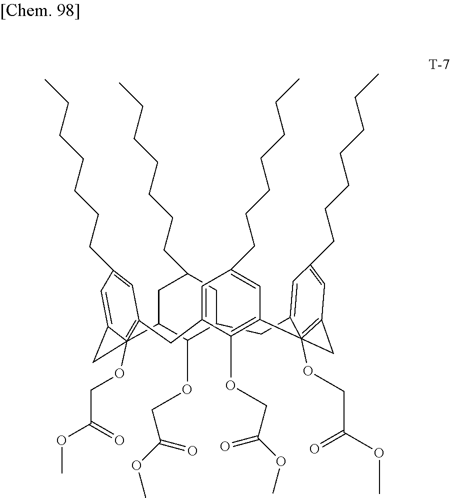

### Synthesis Example 27

The same procedure as in Synthesis Example 24 was repeated except that K-18 was used instead of K-6, to thereby obtain 19.32 g of the target compound T-18. The yield was 80.4%.

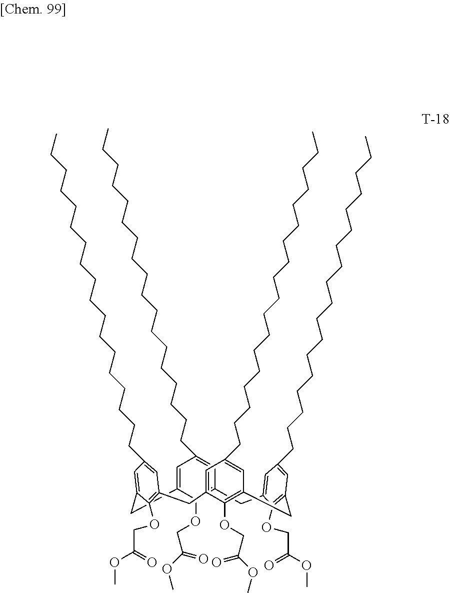

### Synthesis Example 28

The same procedure as in Synthesis Example 24 was repeated except that K-1 was used instead of K-6, to thereby obtain 18.32 g of the target compound T-1. The yield was 57.3%.

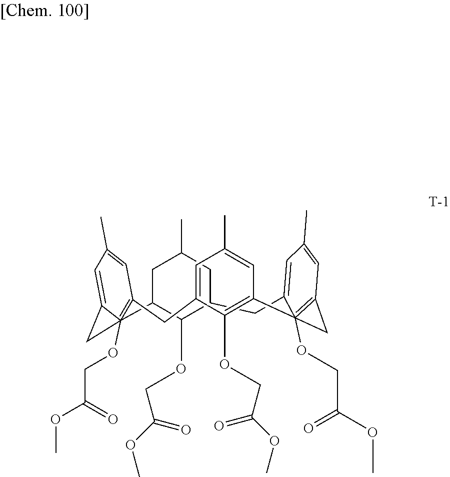

### Synthesis Example 29

A 500 mL four-necked flask equipped with a stirrer, a thermometer, and a reflux condenser tube and placed in an ice bath was charged with 116 mL of dehydrated tetrahydrofuran, and 2.89 g (76.23 mmol) of lithium aluminum hydride was slowly added. 10.00 g (9.529 mmol) of T-6 diluted with 38.6 mL of dehydrated tetrahydrofuran was added from a dropping funnel such that the temperature did not exceed 10° C. The resulting gray suspension reaction solution was allowed to react at room temperature for 6 hours. 100 g of chloroform was added to the resulting solution placed in an ice bath, and 5N hydrochloric acid was added dropwise until the pH was 1 to stop the reaction. Then diatomaceous earth was used to filter the reaction solution, and the filtrate was transferred to a separatory funnel to separate the organic layer. Next, the aqueous layer was extracted with 50 g of chloroform three times, and the extract was combined with the organic layer. The organic layer was pre-dried over anhydrous magnesium sulfate, and the solvent was removed by evaporation using an evaporator. The obtained light yellow liquid was subjected to column chromatography (eluent: n-hexane:ethyl acetate=1:1) to remove by-products and then purified by column chromatography using chloroform:isopropyl alcohol=5:1) to thereby obtain 6.12 g of the target compound U-6 as white crystals. The yield was 68.5%.

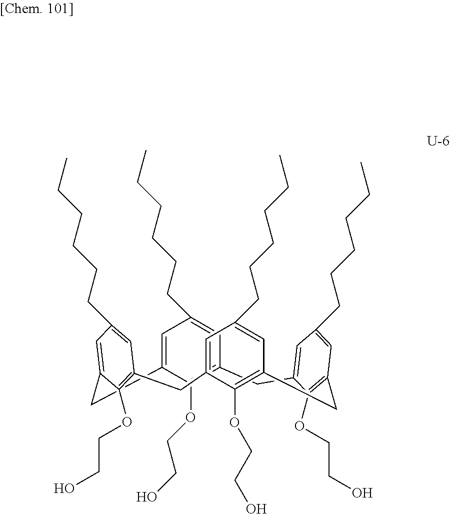

### Synthesis Example 30

The same procedure as in Synthesis Example 29 was repeated except that T-4 was used instead of T-6, to thereby obtain 4.21 g of the target compound U-4. The yield was 81.4%.

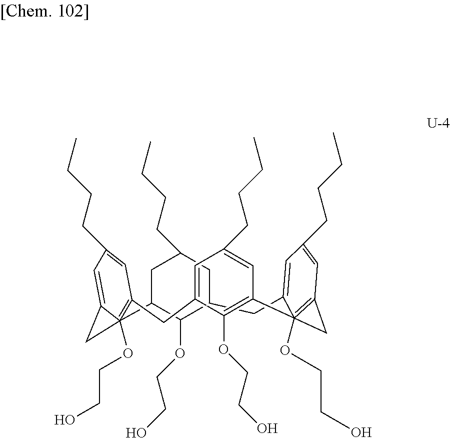

### Synthesis Example 31

The same procedure as in Synthesis Example 29 was repeated except that T-7 was used instead of T-6, to thereby obtain 3.89 g of the target compound U-7. The yield was 84.5%.

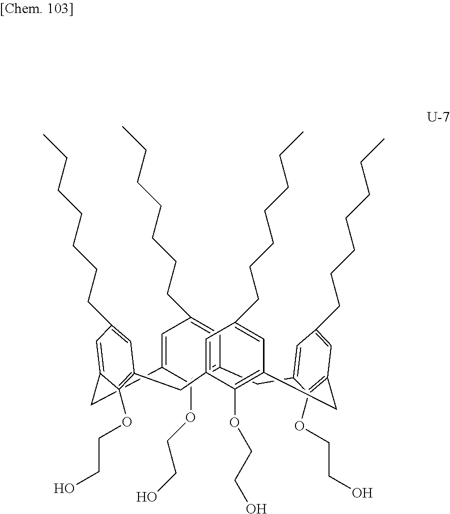

### Synthesis Example 32

The same procedure as in Synthesis Example 29 was repeated except that T-18 was used instead of T-6, to thereby obtain 4.31 g of the target compound U-18. The yield was 81.7%.

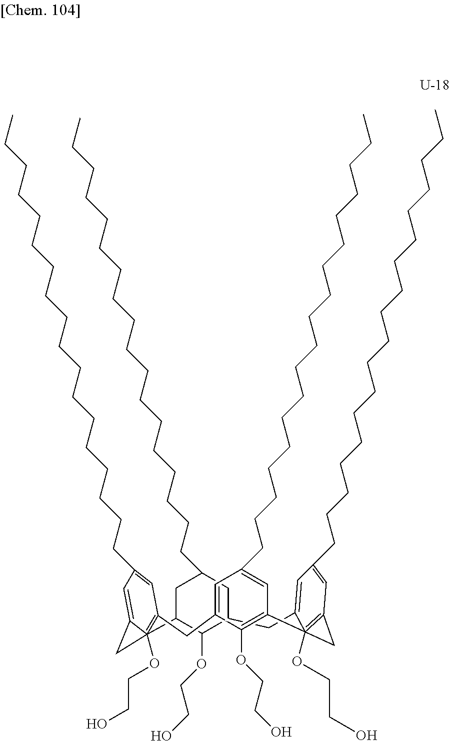

### Synthesis Example 33

The same procedure as in Synthesis Example 29 was repeated except that T-1 was used instead of T-6, to thereby obtain 3.43 g of the target compound U-1. The yield was 85.1%.

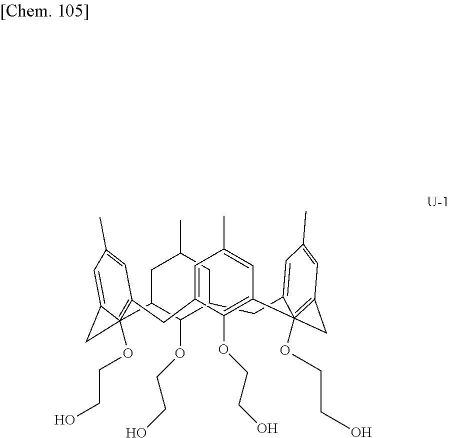

### Synthesis Example 34

A 50 mL four-necked flask equipped with a stirrer, a thermometer, and a reflux condenser tube was charged with 2.00 g (2.424 mmol) of U-6, 10.00 g of tetrahydrofuran, 1.272 g (4.848 mmol) of triphenylphosphine, and 1.024 g (4.732 mmol) of 2-[[[(1,1-dimethylethyl)dimethylsilyl]oxy]-2-propenoic acid, and the mixture was stirred. A light yellow transparent solution. Next, 0.9803 g (4.848 mmol) of diisopropyl azodicarboxylate was added dropwise to the resulting solution placed in an ice bath over 30 minutes. A light yellow transparent solution. The solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the obtained red viscous liquid was purified by column chromatography (eluent: n-hexane:acetone=95:5) to thereby obtain a light yellow transparent liquid. Chloroform/methanol were added to reprecipitate crystals, and the generated white crystals were filtered and vacuum dried (at 60° C. for 6 hours or longer) to thereby obtain 1.891 g of the target compound V-6. The yield was 48.2%.

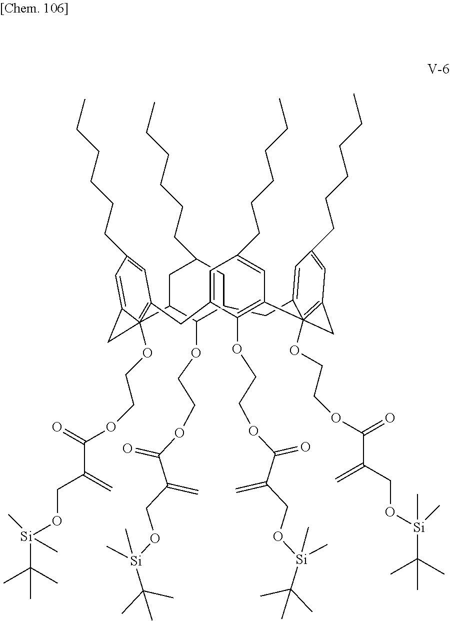

### Synthesis Example 35

The same procedure as in Synthesis Example 34 was repeated except that U-4 was used instead of U-6, to thereby obtain 1.641 g of the target compound V-4. The yield was 57.3%.

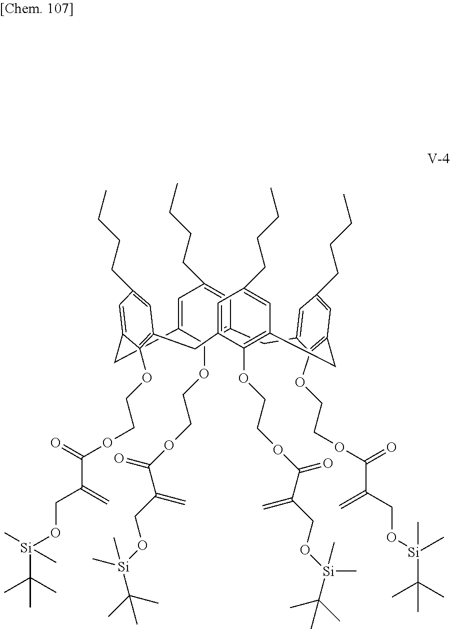

### Synthesis Example 36

The same procedure as in Synthesis Example 34 was repeated except that U-7 was used instead of U-6, to thereby obtain 1.880 g of the target compound V-7. The yield was 79.0%.

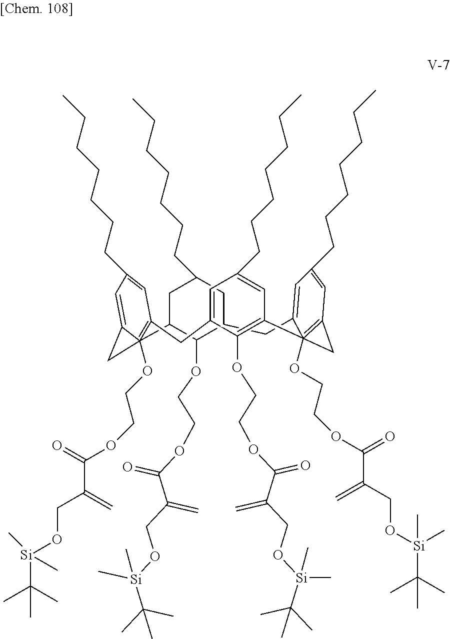

### Synthesis Example 37

The same procedure as in Synthesis Example 34 was repeated except that U-18 was used instead of U-6, to thereby obtain 2.132 g of the target compound V-18. The yield was 71.4%.

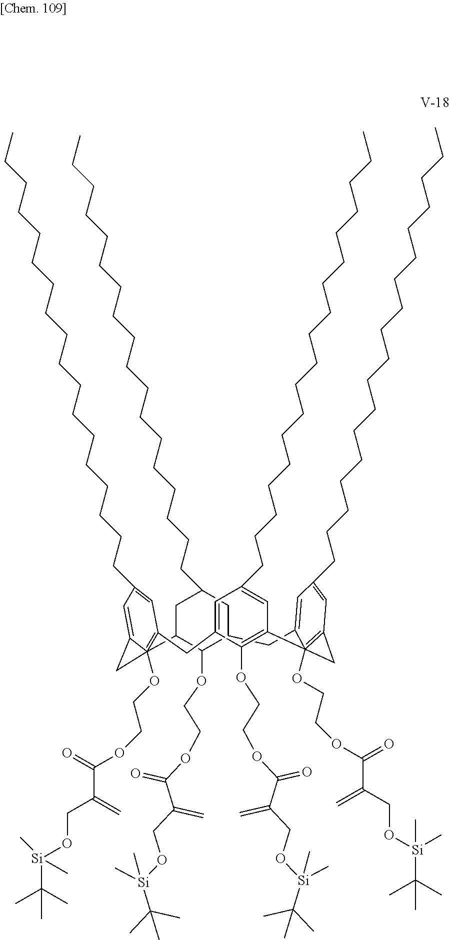

### Synthesis Example 38

The same procedure as in Synthesis Example 34 was repeated except that U-1 was used instead of U-6, to thereby obtain 1.762 g of the target compound V-1. The yield was 39.9%.

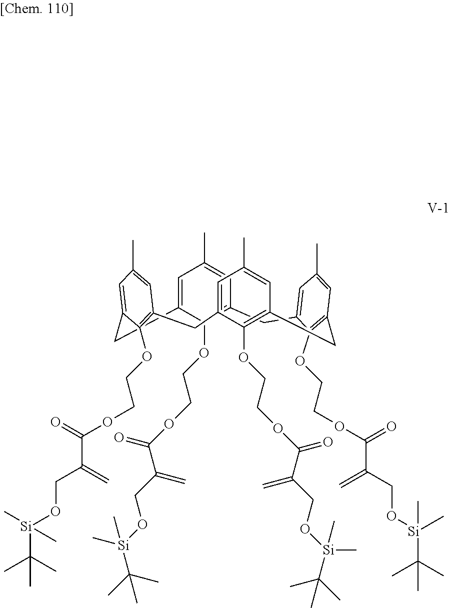

### Synthesis Example 39

A 100 mL four-necked flask equipped with a stirrer, a thermometer, and a reflux condenser tube was charged with 1.891 g (1.168 mmol) of V-6, 50.00 g of tetrahydrofuran, and 0.3367 g (5.606 mmol) of acetic acid, and the mixture was stirred. A colorless transparent solution. Then tetrabutylammonium fluoride (an about 1 mol/L tetrahydrofuran solution 5.61 mL (5.61 mmol) was added dropwise slowly to the resulting solution placed in an ice bath under stirring. The resulting light yellow transparent reaction solution was stirred at room temperature for 6 hours. Ion exchanged water was added to the resulting solution placed in an ice bath, and 30 g of chloroform was added. The reaction mixture was transferred to a separatory funnel, and the organic layer was separated. Next, the aqueous layer was extracted with 30 g of chloroform three times, and the extract was combined with the organic layer. The organic layer was pre-dried over anhydrous magnesium sulfate and filtered. The solvent was removed by evaporation using an evaporator, and the obtained red transparent liquid was purified by column chromatography (eluent: n-hexane:acetone=95:5) to thereby obtain a light yellow transparent liquid. Chloroform/methanol were added to reprecipitate crystals, and the obtained white crystals were vacuum dried (at 60° C. for 6 hours or longer) to thereby obtain 0.8451 g of the target compound W-6. The yield was 62.3%.

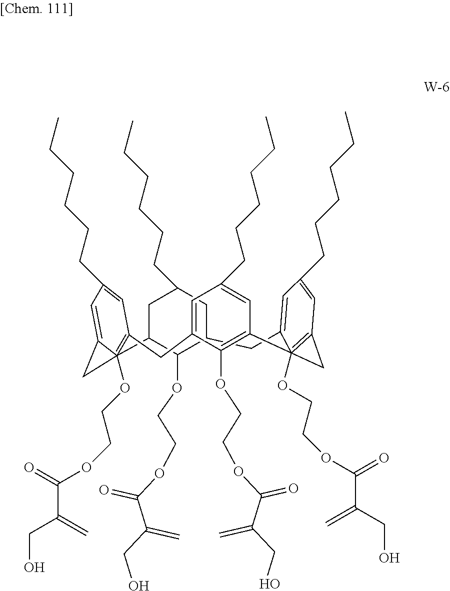

### Synthesis Example 40

The same procedure as in Synthesis Example 39 was repeated except that V-4 was used instead of V-6, to thereby obtain 0.639 g of the target compound W-4. The yield was 54.3%.

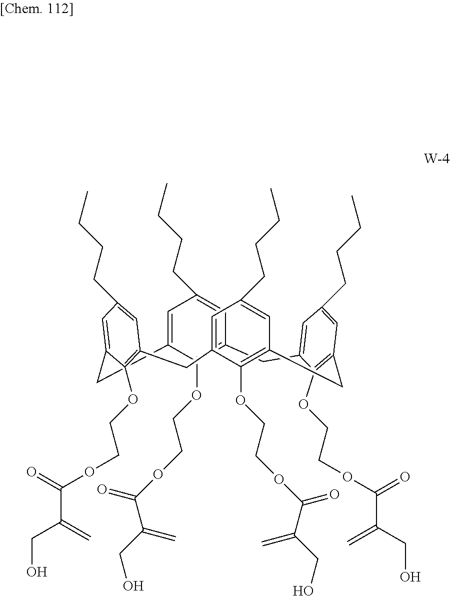

### Synthesis Example 41

The same procedure as in Synthesis Example 39 was repeated except that V-7 was used instead of V-6, to thereby obtain 0.873 g of the target compound W-7. The yield was 62.4%.

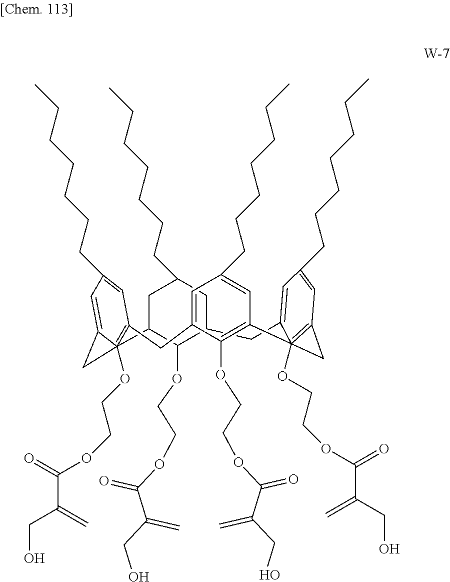

### Synthesis Example 42

The same procedure as in Synthesis Example 39 was repeated except that V-18 was used instead of V-6, to thereby obtain 1.092 g of the target compound W-18. The yield was 63.2%.

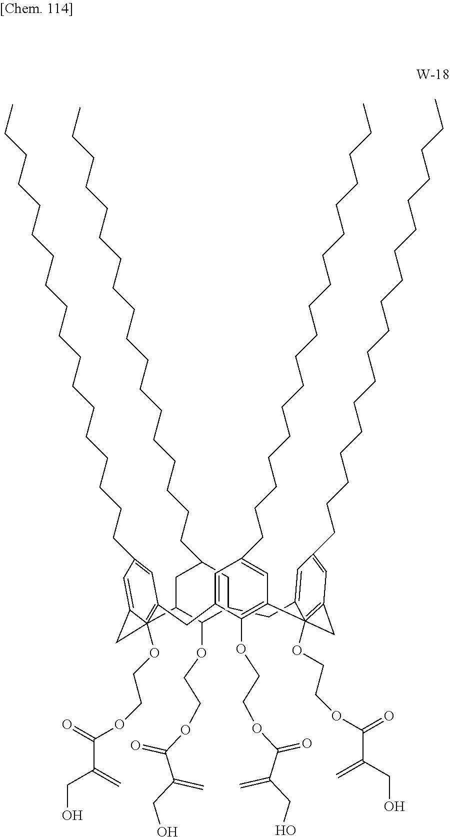

### Synthesis Example 43

The same procedure as in Synthesis Example 39 was repeated except that V-1 was used instead of V-6, to thereby obtain 0.654 g of the target compound W-1. The yield was 54.2%.

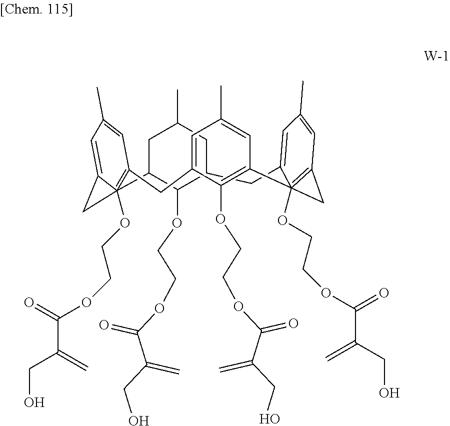

### Example 45

A 50 mL four-necked flask equipped with a stirrer, a thermometer, and a reflux condenser tube was charged with 0.845 g (0.6634 mmol) of W-6, 2.4 g of tetrahydrofuran, 0.766 g (2.919 mmol) of triphenylphosphine, and 0.248 g (2.919 mmol) of acetone cyanohydrin, and the mixture was stirred. Then 0.656 g (2.919 mmol) of diisopropyl azodicarboxylate was added dropwise to the mixture placed in an ice bath over 30 minutes. The resulting light yellow transparent reaction solution was stirred at room temperature for 48 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the obtained red viscous liquid was purified by column chromatography (eluent: n-hexane:acetone=90:10) to thereby obtain a light yellow transparent liquid. Chloroform/methanol were added to reprecipitate crystals, and the obtained white crystals were vacuum dried (at 60° C. for 6 hours or longer) to thereby obtain 0.398 g of the target compound 33-6. The yield was 45.8%.

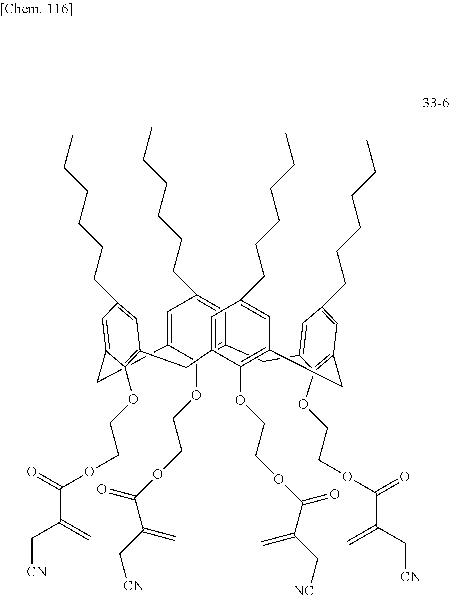

### Example 46

The same procedure as in Example 45 was repeated except that W-4 was used instead of W-6, to thereby obtain 0.265 g of the target compound 33-4. The yield was 40.2%.

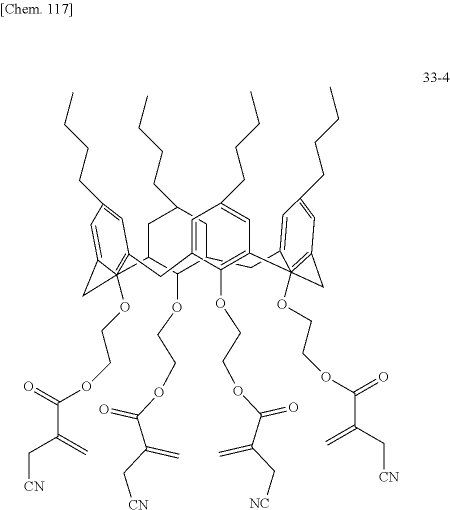

### Example 47

The same procedure as in Example 45 was repeated except that W-7 was used instead of W-6, to thereby obtain 0.465 g of the target compound 33-7. The yield was 51.9%.

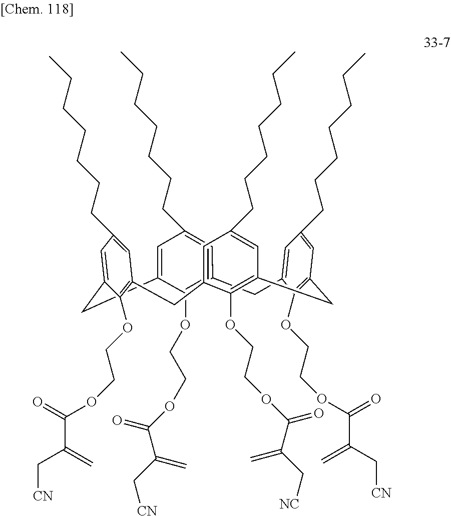 [SMILES: C=C(CC#N)C(=O)OCCOc1c2cc(CCCCCCC)cc1Cc1cc(CCCCCCC)cc(c1OCCOC(=O)C(=C)CC#N)Cc1cc(CCCCCCC)cc(c1OCCOC(=O)C(=C)CC#N)Cc1cc(CCCCCCC)cc(c1OCCOC(=O)C(=C)CC#N)C2]

### Example 48

The same procedure as in Example 45 was repeated except that W-18 was used instead of W-6, to thereby obtain 0.669 g of the target compound 33-7. The yield was 60.2%.

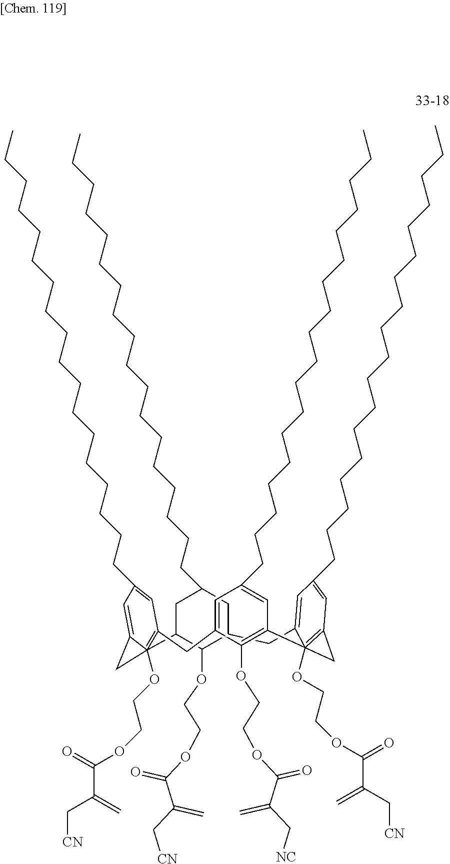

### Example 49

The same procedure as in Example 45 was repeated except that W-7 was used instead of W-6, to thereby obtain 0.257 g of the target compound 33-1. The yield was 37.9%.

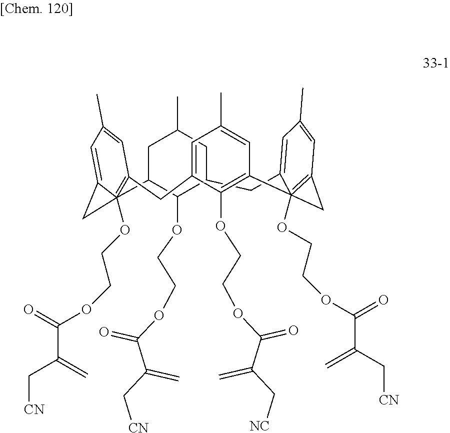

### Synthesis Example 44

A 100 mL four-necked flask equipped with a stirrer, a thermometer, and a reflux condenser tube was charged with 2.00 g (1.570 mmol) of U-6, 6.8 g of tetrahydrofuran, 0.824 g (3.141 mmol) of triphenylphosphine, and 0.706 g (3.065 mmol) of 4-[[[(1,1-dimethylethyl)dimethylsilyl]oxy]-2-methylenebutanoic acid, and the mixture was stirred. A light yellow transparent solution. 0.635 g (3.140 mmol) of diisopropyl azodicarboxylate was added dropwise to the solution placed in an ice bath over 30 minutes. The resulting light yellow transparent reaction solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:acetone=95:5) to thereby obtain a light yellow transparent liquid.

Chloroform/methanol were added to reprecipitate crystals, and the obtained white crystals were vacuum dried (at 60° C. for 6 hours or longer) to thereby obtain 2.420 g of the target compound X-6. The yield was 72.6%.

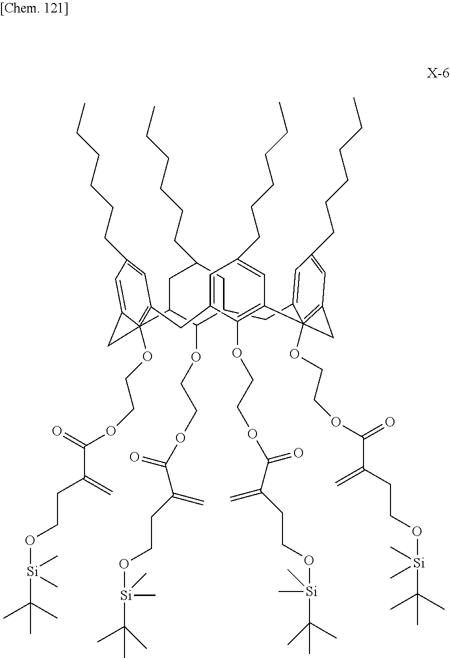

### Synthesis Example 45

The same procedure as in Synthesis Example 39 was repeated except that X-1 was used instead of V-6, to thereby obtain 1.07 g of the target compound Y-6. The yield was 59.4%.

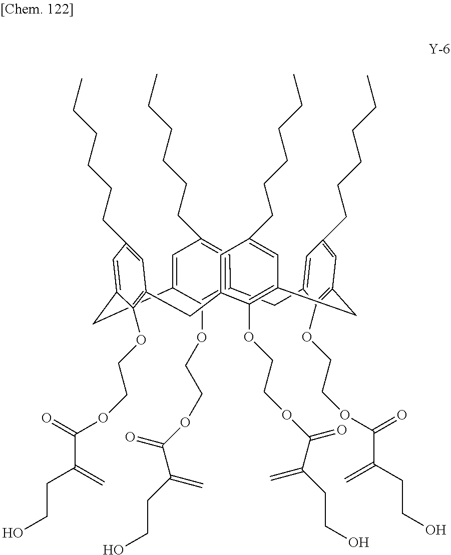 [SMILES: C=C(CCO)C(=O)OCCOc1c2cc(CCCCCC)cc1Cc1cc(CCCCCC)cc(c1OCCOC(=O)C(=C)CCO)Cc1cc(CCCCCC)cc(c1OCCOC(=O)C(=C)CCO)Cc1cc(CCCCCC)cc(c1OCCOC(=O)C(=C)CCO)C2]

### Example 50

The same procedure as in Example 45 was repeated except that Y-6 was used instead of W-6, to thereby obtain 0.577 g of the target compound 34-6. The yield was 52.5%.

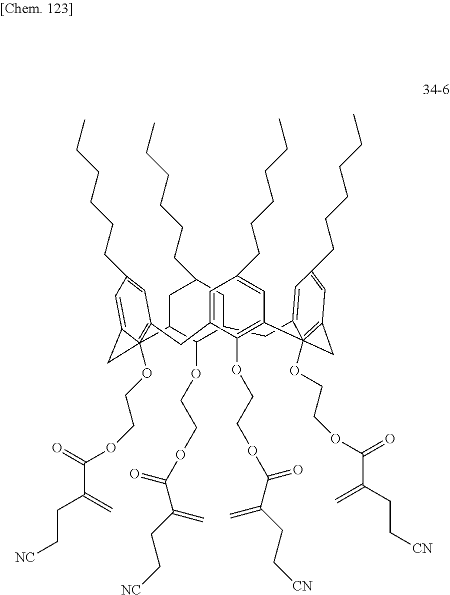

### Synthesis Example 46

A 50 mL four-necked flask equipped with a stirrer, a thermometer, and a reflux condenser tube was charged with 2.00 g (1.570 mmol) of G-6, 6.8 g of tetrahydrofuran, 0.905 g (3.454 mmol) of triphenyiphosphine, and 0.304 g (3.454 mmol) of hydroxyethyl vinyl ether, and the mixture was stirred. A light yellow transparent solution. Then 0.698 g (3.454 mmol) of diisopropyl azodicarboxylate was added dropwise to the solution placed in an ice bath over 30 minutes. The resulting light yellow transparent reaction solution was stirred at room temperature for 6 hours. Hexane was added to the reaction solution to precipitate and remove by-products such as triphenylphosphine. Then the resulting solution was extracted with chloroform, washed with water and saturated brine, and dried over magnesium sulfate. The solvent was removed by evaporation using an evaporator, and the obtained orange viscus liquid was purified by column chromatography (eluent: n-hexane:acetone=90:10) to thereby obtain 0.756 g of the target compound Z-6. The yield was 38.9%.

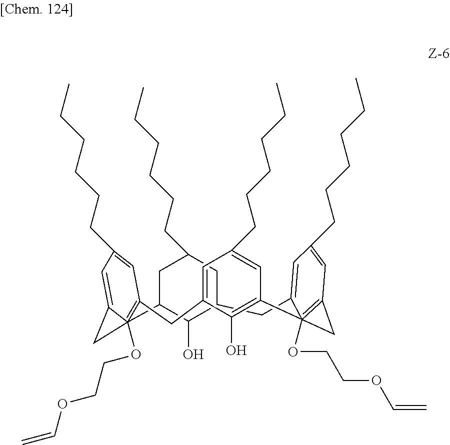

### Example 51

The same procedure as in Example 1 was repeated except that Z-6 was used instead of B-6, to thereby obtain 0.442 g of the target compound 35-6. The yield was 52.3%.

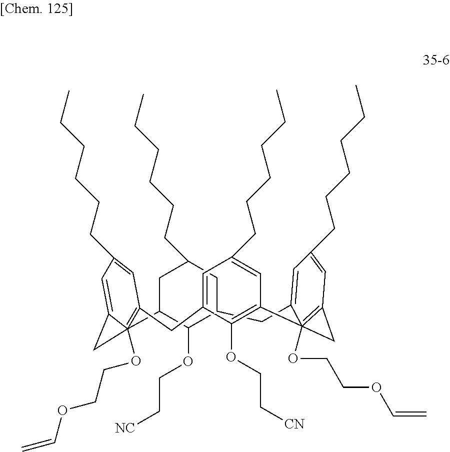

### Synthesis Example 47

A 1 L four-necked flask equipped with a stirrer, a dropping funnel, a thermometer, and a reflux condenser tube was charged with sodium hydride (7.54 g, 188.4 mmol) in a nitrogen atmosphere, and mineral oil was removed by washing with hexane. Then DMF (160 mL) and 37.2 g of hexyl bromide (207.4 mmol) were added, and the mixture was heated to 70° C. under stirring. A solution prepared by dissolving intermediate A (10 g, 23.6 mmol) obtained in Synthesis Example 1 in DMF (80 mL) was added to the resulting solution from a dropping funnel, and the mixture was stirred for 2 hours. After cooled to room temperature, the reaction mixture was poured onto ice (300 g). Concentrated hydrochloric acid was added to acidify the mixture, and the resulting mixture was extracted with chloroform (200 mL) twice. The chloroform solution was washed with water until the pH was 5 or higher, further washed with saturated brine, and dried over anhydrous magnesium sulfate. The solvent was removed using an evaporator to thereby obtain a yellow liquid. Methanol was added to the mixture under stirring to precipitate solids. The solids were collected by filtration and recrystallized with isopropyl alcohol. The obtained white crystals were vacuum dried to thereby obtain a compound represented by the following formula (11.6 g, yield 65%).

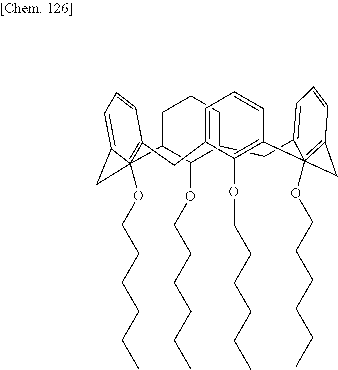

### Synthesis Example 48

The same procedure as in Synthesis Example 47 was repeated except that methyl iodide was used instead of hexyl bromide and that the reaction was performed at room temperature for 24 hours, to thereby obtain a compound represented by the following formula (6.8 g, yield 60%).

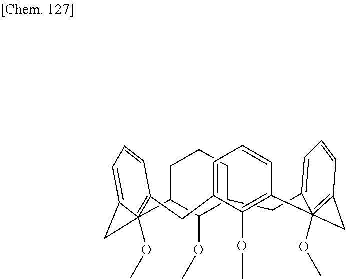

### Synthesis Example 49

The same procedure as in Synthesis Example 47 was repeated except that butyl bromide was used instead of hexyl bromide, to thereby obtain a compound represented by the following formula (11.0 g, yield 72%).

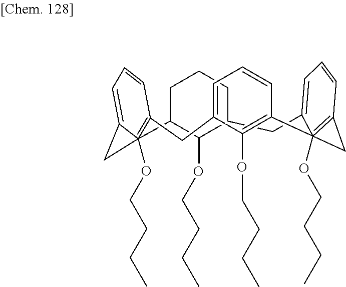

### Synthesis Example 50

The same procedure as in Synthesis Example 47 was repeated except that heptyl bromide was used instead of hexyl bromide, to thereby obtain a compound represented by the following formula (14.4 g, yield 75%).

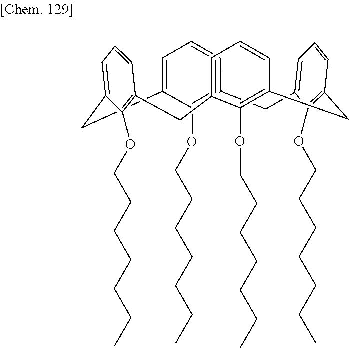 [SMILES: CCCCCCCOc1c2cccc1Cc1cccc(c1OCCCCCCC)Cc1cccc(c1OCCCCCCC)Cc1cccc(c1OCCCCCCC)C2]

### Synthesis Example 51

The same procedure as in Synthesis Example 47 was repeated except that octadecyl bromide was used instead of hexyl bromide, to thereby obtain a compound represented by the following formula (23.6 g, yield 70%).

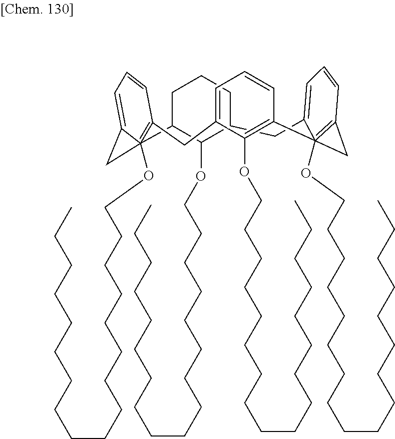

### Synthesis Example 52

Referring to a known document (Organic & Biomolecular Chemistry, 13, 1708-1723; 2015), a compound represented by the following formula was synthesized in two steps using the compound obtained in Synthesis Example 47 (5.0 g, 6.57 mmol) (yield amount 3.3 g, yield 67%)

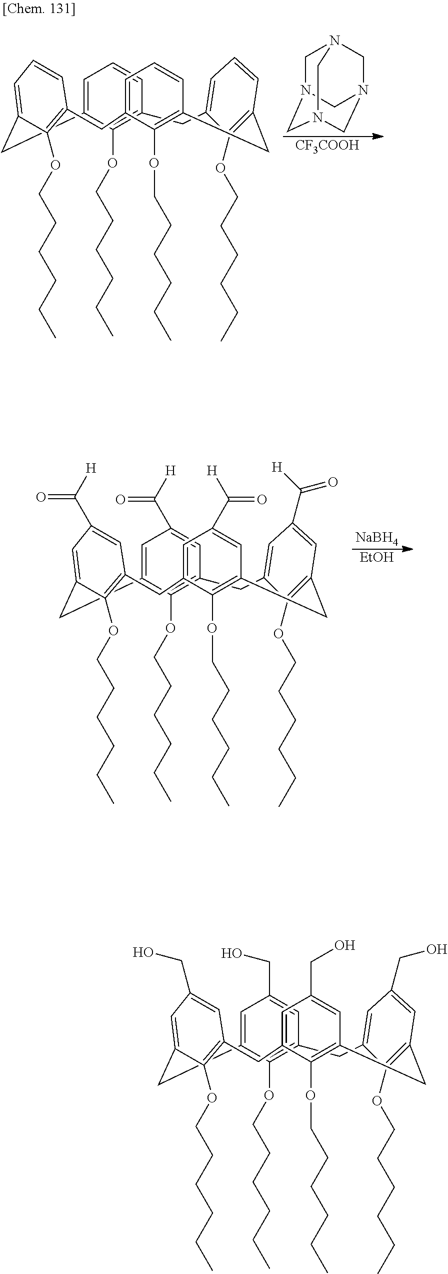

### Synthesis Example 53

The same procedure as in Synthesis Example 52 was repeated except that the compound obtained in Example 48 (5.0 g, 10.4 mmol) was used instead of the compound obtained in Synthesis Example 47, to synthesize a compound represented by the following formula in two steps (3.75 g, yield 60%).

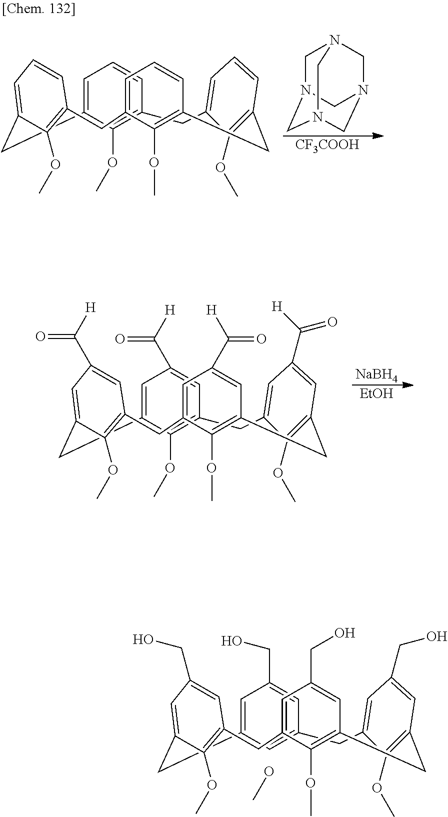

### Synthesis Example 54

The same procedure as in Synthesis Example 52 was repeated except that the compound obtained in Synthesis Example 49 (5.0 g, 7.7 mmol) was used instead of the compound obtained in Synthesis Example 47, to synthesize a compound represented by the following formula in two steps (3.73 g, yield 63%).

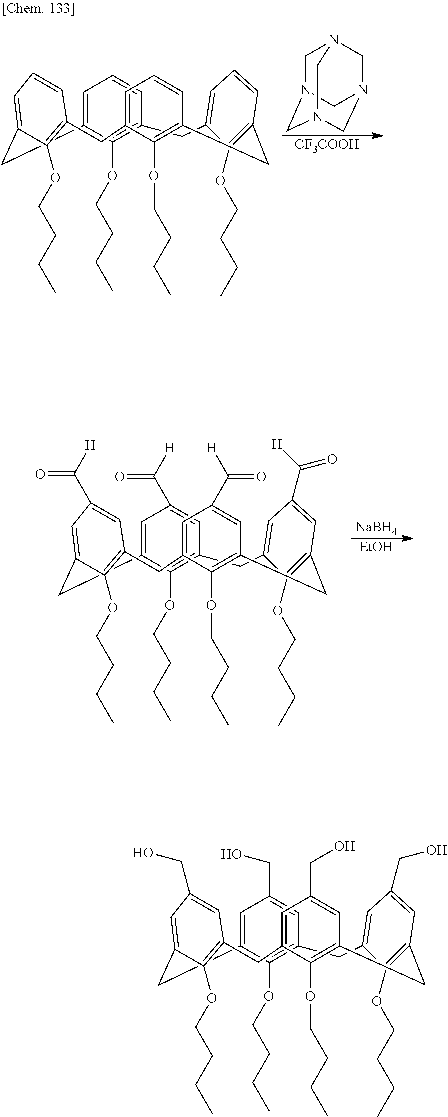

### Synthesis Example 55

The same procedure as in Synthesis Example 52 was repeated except that the compound obtained in Synthesis Example 50 (5.0 g, 6.1 mmol) was used instead of the compound obtained in Synthesis Example 47, to synthesize a compound represented by the following formula in two steps (4.01 g, yield 70%).

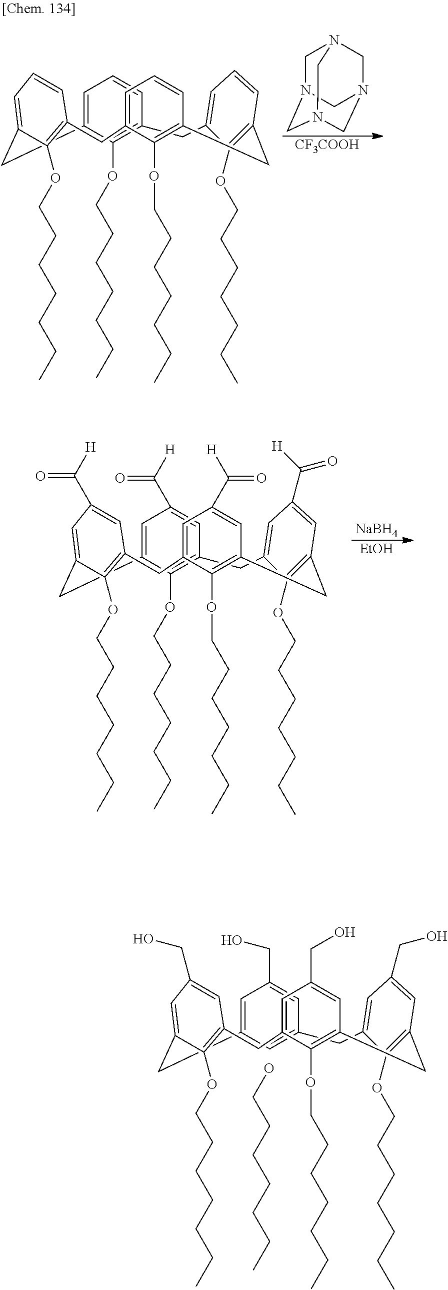

### Synthesis Example 56

The same procedure as in Synthesis Example 52 was repeated except that the compound obtained in Synthesis Example 51 (10.0 g, 7.0 mmol) was used instead of the compound obtained in Synthesis Example 47, to synthesize a compound represented by the following formula in two steps (5.96 g, yield 55%).

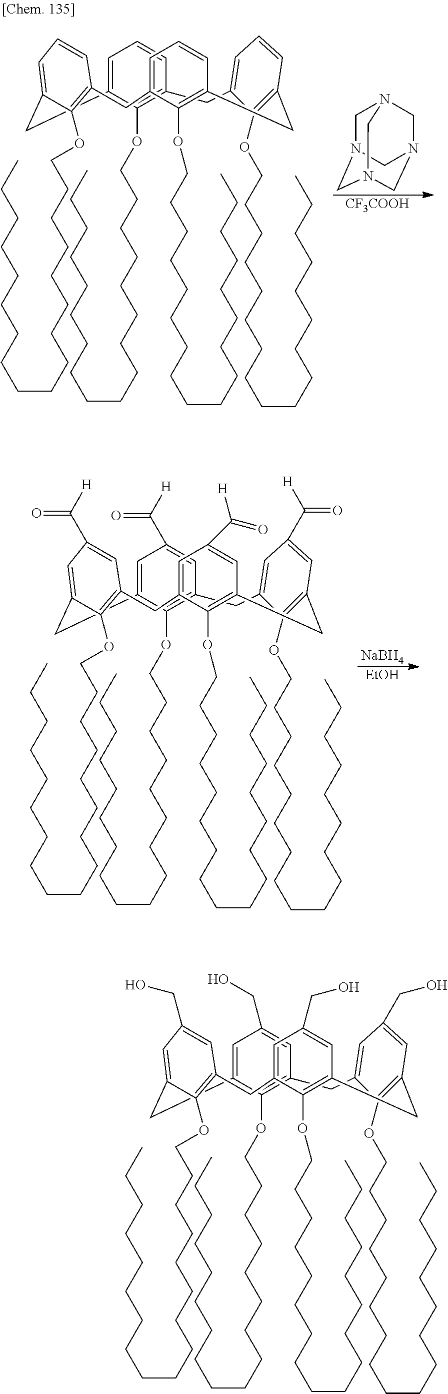

### Synthesis Example 57

A 500 mL four-necked flask equipped with a stirrer, a dropping funnel, a thermometer, and a reflux condenser tube was charged with sodium hydride (3.28 g, 82.1 mmol) in a nitrogen atmosphere, and mineral oil was removed by washing with hexane. Next, dry DMF (100 mL) and hexyl bromide (16.2 g, 90.3 mmol) were added, and the mixture was heated to 70° C. under stirring. Then a solution prepared by dissolving in dry DMF (40 mL) 5,11,17,23-tetraallyl-25,26,27,28-tetrahydroxycalix[4]arene (6.0 g, 10.3 mmol) synthesized by a method described in a known document (The Journal of Organic Chemistry 50, 5802-58061; 1985) was added from a dropping funnel. After completion of the addition, the mixture was continuously stirred for additional 2 hours. The reaction mixture was cooled to room temperature and then poured onto ice (200 g). Concentrated hydrochloric acid was added to acidify the aqueous solution, and the resulting solution was extracted with chloroform (150 mL) twice. The chloroform solution was washed with water until the pH was 5 or higher, further washed with saturated brine, and dried over anhydrous magnesium sulfate. The solvent was removed using an evaporator, and a yellow liquid was thereby obtained. The yellow liquid was purified by silica gel column chromatography to thereby obtain a colorless transparent solution, and a compound represented by the following formula was obtained as a colorless transparent solution by recrystallization (6.6 g, yield 70%).

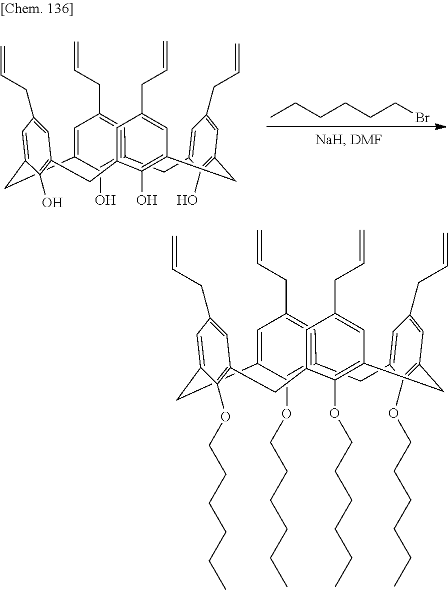

### Synthesis Example 58

The same procedure as in Synthesis Example 57 was repeated except that methyl iodide was used instead of hexyl bromide and that the reaction was performed at room temperature for 24 hours, to thereby obtain a compound represented by the following formula (4.27 g, yield 65%).

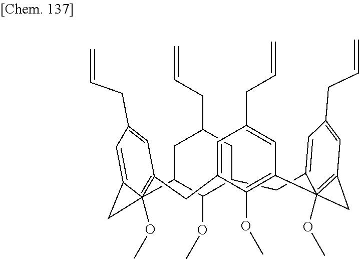

### Synthesis Example 59

The same procedure as in Synthesis Example 57 was repeated except that butyl bromide was used instead of hexyl bromide, to thereby obtain a compound represented by the following formula (6.23 g, yield 75%).

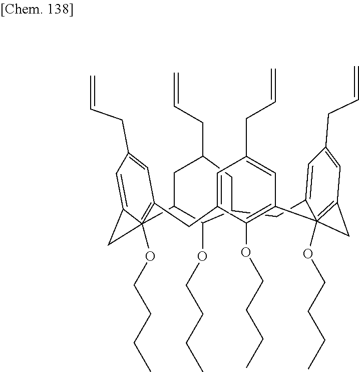

### Synthesis Example 60

The same procedure as in Synthesis Example 57 was repeated except that heptyl bromide was used instead of hexyl bromide, to thereby obtain a compound represented by the following formula (8.02 g, yield 80%).

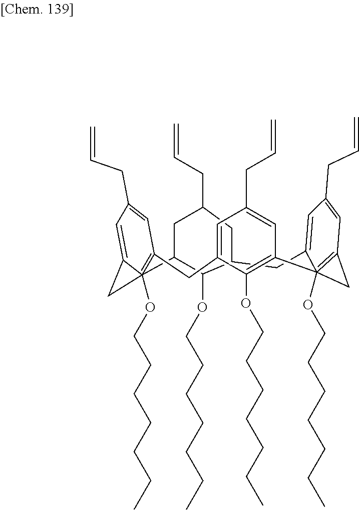

### Synthesis Example 61

The same procedure as in Synthesis Example 57 was repeated except that octadecyl bromide was used instead of hexyl bromide, to thereby obtain a compound represented by the following formula (12.8 g, yield 75%).

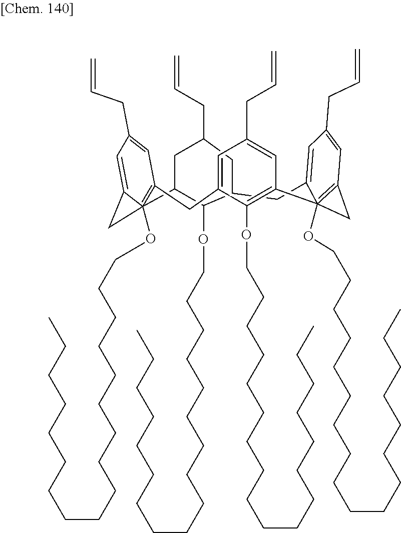

### Synthesis Example 62

Referring to a known document (The Journal of Organic Chemistry, 67, 4722-4733; 2002), a compound represented by the following formula was synthesized using the compound obtained in Synthesis Example 57 (4 g, 4.34 mmol) (yield amount 2.93 g, yield 68%).

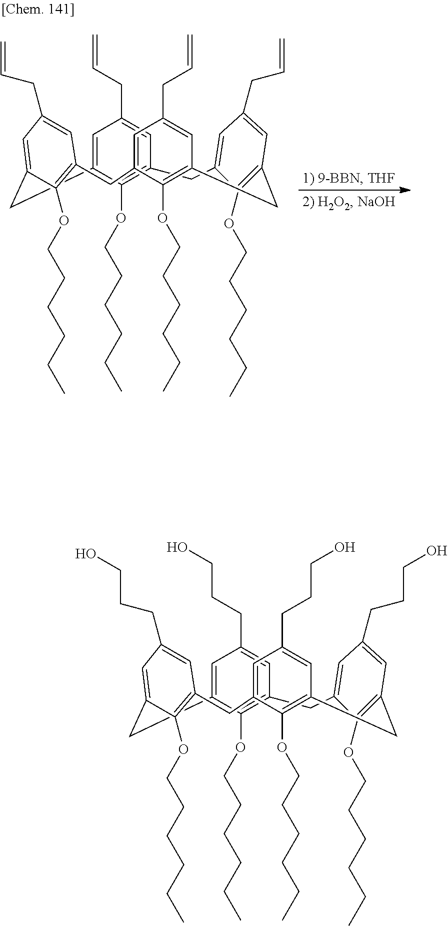 [SMILES: C=CCc1cc2c(OCCCCCC)c(c1)Cc1cc(CC=C)cc(c1OCCCCCC)Cc1cc(CC=C)cc(c1OCCCCCC)Cc1cc(CC=C)cc(c1OCCCCCC)C2 ; CCCCCCOc1c2cc(CCCO)cc1Cc1cc(CCCO)cc(c1OCCCCCC)Cc1cc(CCCO)cc(c1OCCCCCC)Cc1cc(CCCO)cc(c1OCCCCCC)C2]

### Synthesis Example 63

The same procedure as in Synthesis Example 62 was repeated except that the compound obtained in Synthesis Example 58 (4.0 g, 6.24 mmol) was used instead of the compound obtained in Synthesis Example 57, to thereby obtain a compound represented by the following formula (4.5 g, yield 72%).

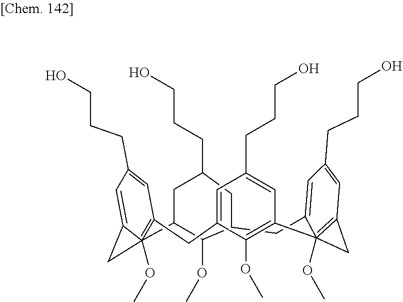

### Synthesis Example 64

The same procedure as in Synthesis Example 62 was repeated except that the compound obtained in Synthesis Example 59 (4.0 g, 4.94 mmol) was used instead of the compound obtained in Synthesis Example 57, to thereby obtain a compound represented by the following formula (2.59 g, yield 65%).

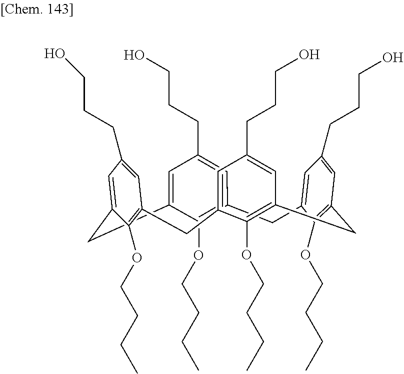 [SMILES: CCCCOc1c2cc(CCCO)cc1Cc1cc(CCCO)cc(c1OCCCC)Cc1cc(CCCO)cc(c1OCCCC)Cc1cc(CCCO)cc(c1OCCCC)C2]

### Synthesis Example 65

The same procedure as in Synthesis Example 62 was repeated except that the compound obtained in Synthesis Example 60 (4.0 g, 4.11 mmol) was used instead of the compound obtained in Synthesis Example 57, to thereby obtain a compound represented by the following formula (3.23 g, yield 75%).

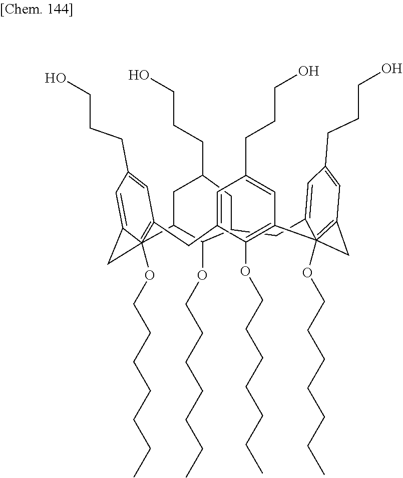

### Synthesis Example 66

The same procedure as in Synthesis Example 62 was repeated except that the compound obtained in Synthesis Example 61 (8.0 g, 5.02 mmol) was used instead of the compound obtained in Synthesis Example 57, to thereby obtain a compound represented by the following formula (5.1 g, yield 61%).

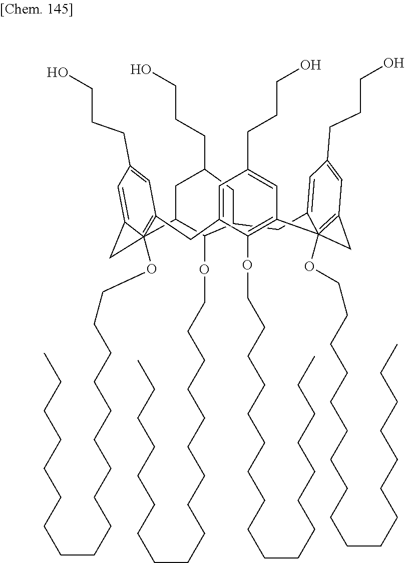

### Example 52

A 100 mL four-necked flask equipped with a stirrer, a dropping funnel, and a thermometer was charged, in a nitrogen atmosphere, with the compound obtained in Synthesis Example 52 (3.0 g, 3.94 mmol), 3.10 g (11.82 mmol) of triphenylphosphine, 1.006 g (11.82 mmol) of acetone cyanohydrin, and 32 mL of tetrahydrofuran, and the mixture was stirred. Next, 2.39 g (11.82 mmol) of diisopropyl azodicarboxylate was added dropwise to the mixture placed in an ice bath over 30 minutes, and the resulting mixture was further stirred at room temperature for 48 hours. The reaction solution was concentrated in an evaporator, and hexane was added to precipitate and remove by-products such as triphenylphosphine. The obtained yellow viscous liquid was used for the next reaction without purification. A 100 mL four-necked flask equipped with a stirrer, a dropping funnel, and a thermometer was charged, in a nitrogen atmosphere, with the crude product obtained above, triethylamine (2.392 g, 23.64 mmol), and methylene chloride (30.0 mL), and the mixture was stirred under cooling with ice. Acrylic acid chloride (1.426 g, 15.76 mmol) was slowly added dropwise. After completion of the dropwise addition, the mixture was stirred at room temperature for 8 hours. Water was added to the reaction mixture, and the resulting mixture was extracted with chloroform (50 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator, and a yellow liquid was thereby obtained. The yellow liquid was purified by silica gel column chromatography to thereby obtain the target compounds 01-6, 02-6, 03-6, and 04-6. 01-6 (0.360 g, yield 9.5%). A mixture of 02-6 and 03-6 (1.925 g, yield 48.5%). 04-6 (0.469 g, yield 11.3%).

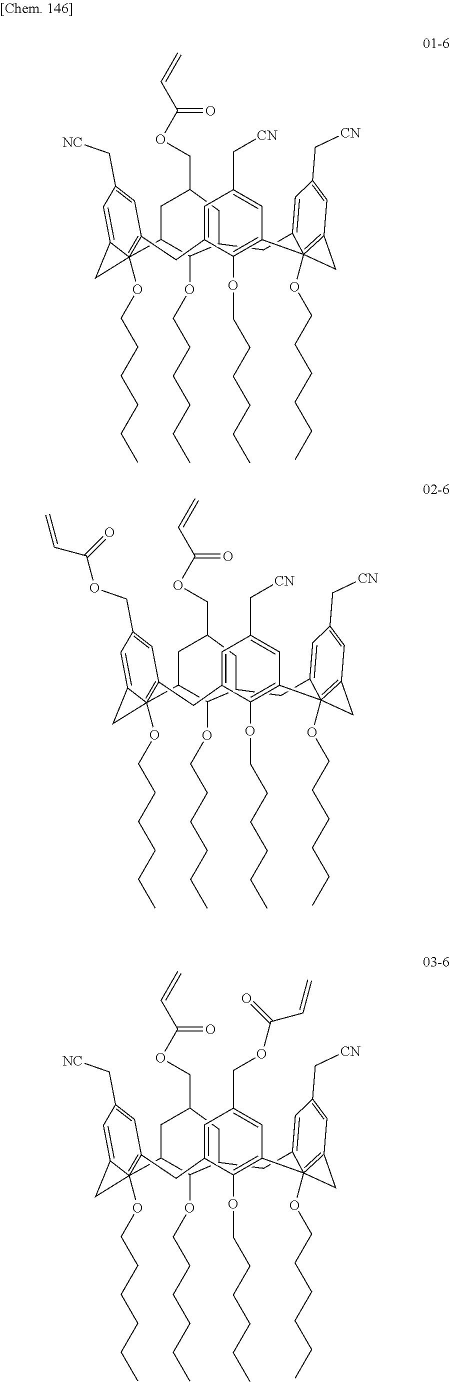

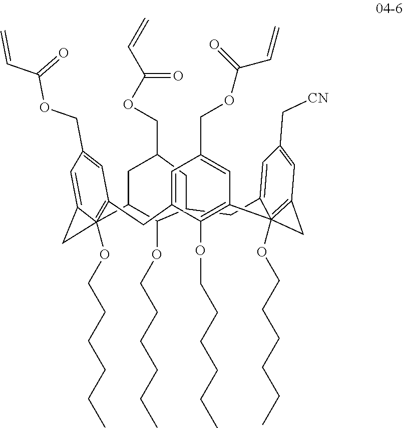

### Example 53

The same procedure as in Example 52 was repeated except that the compound obtained in Synthesis Example 53 (3.0 g, 4.99 mmol) was used instead of the compound obtained in Synthesis Example 52, to thereby obtain the target compounds 01-1, 02-1, 03-1, and 04-1. 01-1 (0.334 g, yield 9.8%). A mixture of 02-1 and 03-1 (1.641 g, yield 45.2%). 04-1 (0.397 g, yield 10.3%).

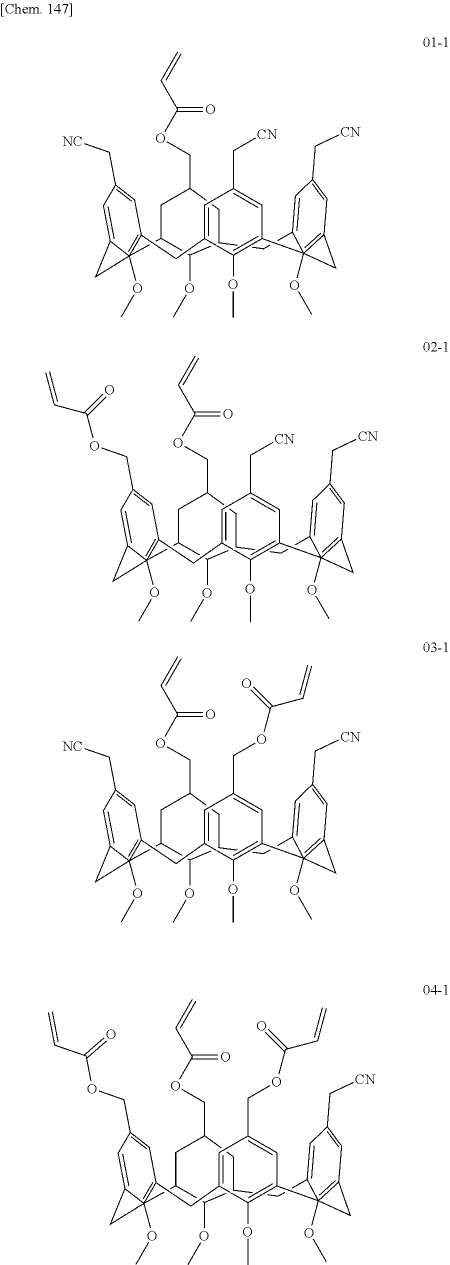

### Example 54

The same procedure as in Example 52 was repeated except that the compound obtained in Synthesis Example 54 (3.0 g, 3.9 mmol) was used instead of the compound obtained in Synthesis Example 52, to thereby obtain the target compounds 01-4, 02-4, 03-4, and 04-4. 01-4 (0.358 g, yield 10.8%). A mixture of 02-4 and 03-4 (1.624 g, yield 46.5%). 04-4 (0.374 g, yield 10.2%).

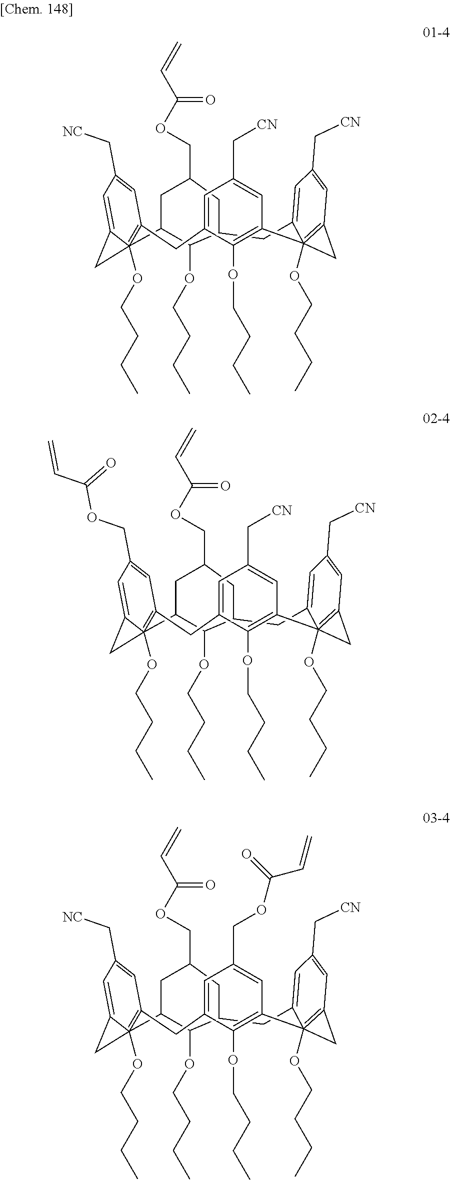

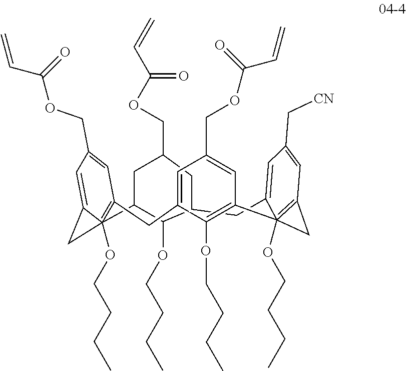

### Example 55

The same procedure as in Example 52 was repeated except that the compound obtained in Synthesis Example 55 (3.0 g, 3.2 mmol) was used instead of the compound obtained in Synthesis Example 52, to thereby obtain the target compounds 01-7, 02-7, 03-7, and 04-7. 01-7 (0.407 g, yield 12.5%). A mixture of 02-7 and 03-7 (1.685 g, yield 49.5%). 04-7 (0.401 g, yield 11.3%).

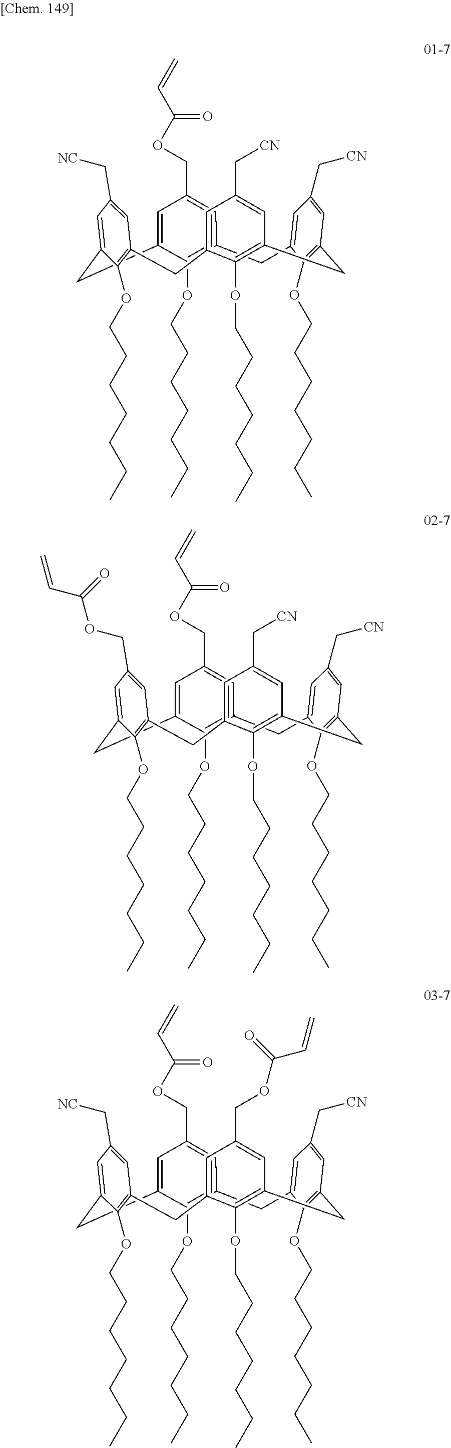

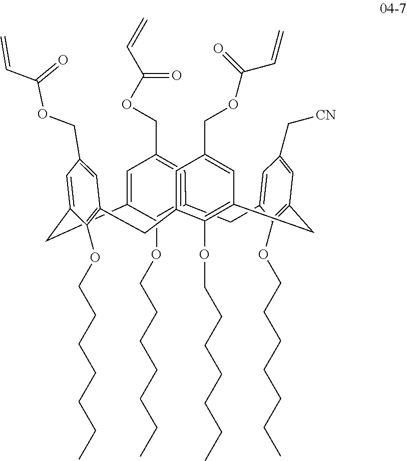 [SMILES: C=CC(=O)OCc1cc2c(OCCCCCCC)c(c1)Cc1cc(COC(=O)C=C)cc(c1OCCCCCCC)Cc1cc(COC(=O)C=C)cc(c1OCCCCCCC)Cc1cc(CC#N)cc(c1OCCCCCCC)C2]

### Example 56

The same procedure as in Example 01 was repeated except that b-18 (3.0 g, 1.93 mmol) was used instead of b-6, to thereby obtain the target compounds 01-18, 02-18, 03-18, and 04-18. 01-18 (0.271 g, yield 8.6%). A mixture of 02-18 and 03-18 (1.55 g, yield 47.8%). 04-18 (0.383 g, yield 11.5%).

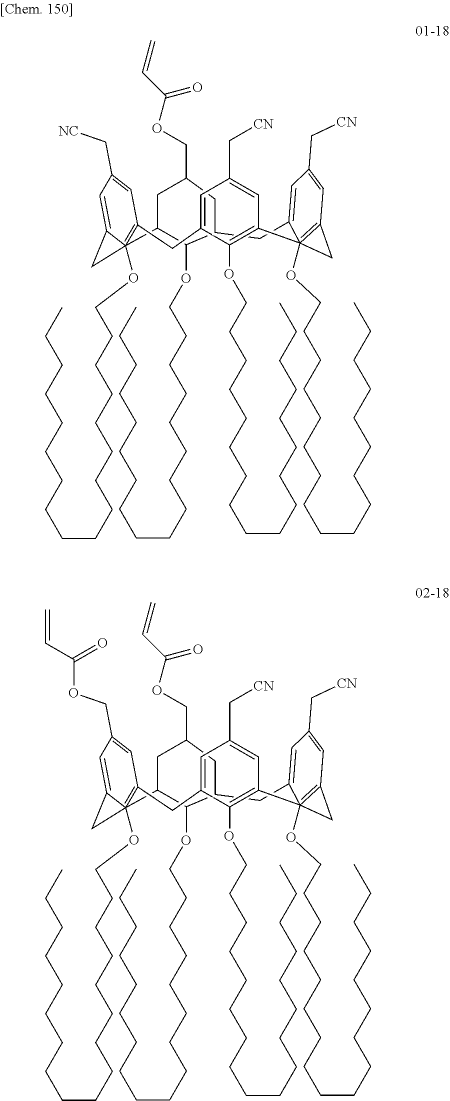

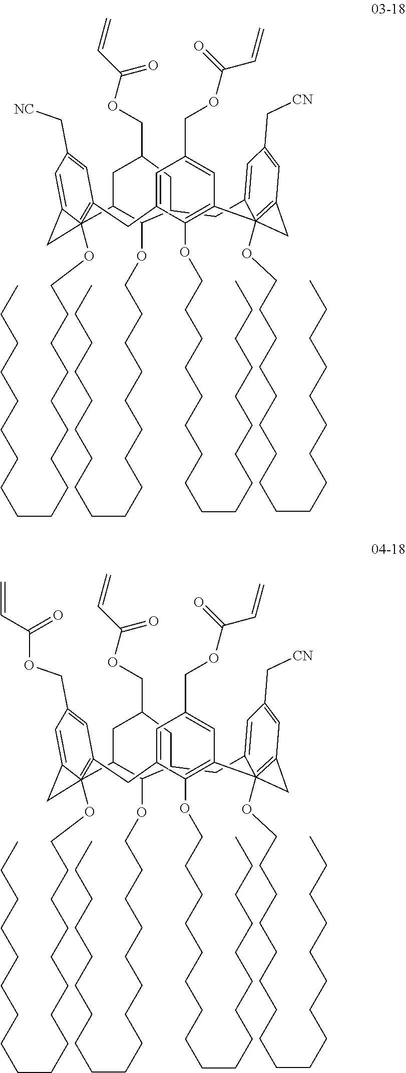

### Synthesis Example 67

A 100 mL four-necked flask equipped with a stirrer, a thermometer, and a reflux condenser tube was charged with 2.00 g (2.27 mmol) of the compound obtained in Synthesis Example 52, 3.57 g (13.62 mmol) of triphenylphosphine, 2.95 g (13.62 mmol) of 2-[([(1,1-dimethylethyl)dimethylsilyl]oxy]2-propenoic acid, and 38 mL of tetrahydrofuran, and the mixture was stirred. Then 2.75 g (13.62 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 2.85 g, yield 75.0%).

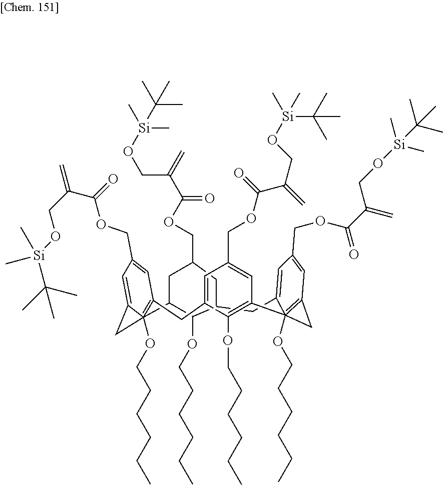

### Synthesis Example 68

The same procedure as in Synthesis Example 67 was repeated except that the compound obtained in Synthesis Example 53 (2.00 g, 3.33 mmol) was used instead of the compound obtained in Synthesis Example 52, to thereby obtain a compound represented by the following formula (3.26 g, yield 70.2%).

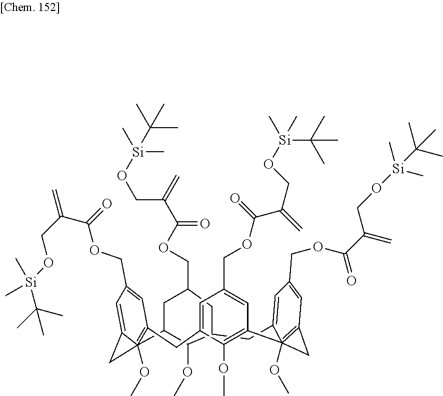

### Synthesis Example 69

The same procedure as in Synthesis Example 67 was repeated except that the compound obtained in Synthesis Example 54 (2.00 g, 2.60 mmol) was used instead of the compound obtained in Synthesis Example 52, to thereby obtain a compound represented by the following formula (3.12 g, yield 76.8%).

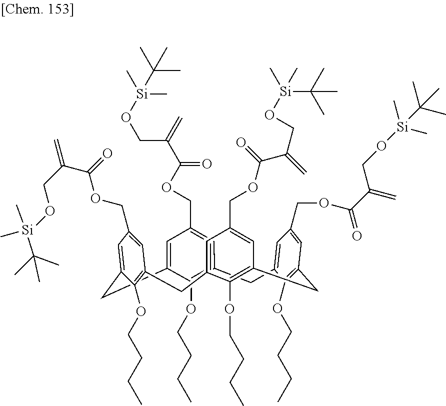 [SMILES: C=C(CO[Si](C)(C)C(C)(C)C)C(=O)OCc1cc2c(OCCCC)c(c1)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)C2]

### Synthesis Example 70

The same procedure as in Synthesis Example 67 was repeated except that the compound obtained in Synthesis Example 55 (2.00 g, 2.13 mmol) was used instead of the compound obtained in Synthesis Example 52, to thereby obtain a compound represented by the following formula (2.74 g, yield 74.2%).

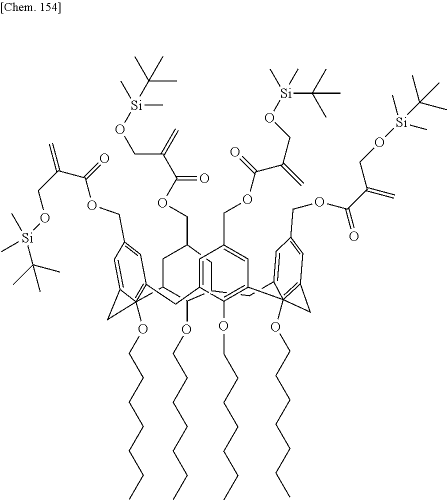

### Synthesis Example 71

The same procedure as in Synthesis Example 67 was repeated except that the compound obtained in Synthesis Example 56 (2.00 g, 1.29 mmol) was used instead of the compound obtained in Synthesis Example 52, to thereby obtain a compound represented by the following formula (2.58 g, yield 85.3%).

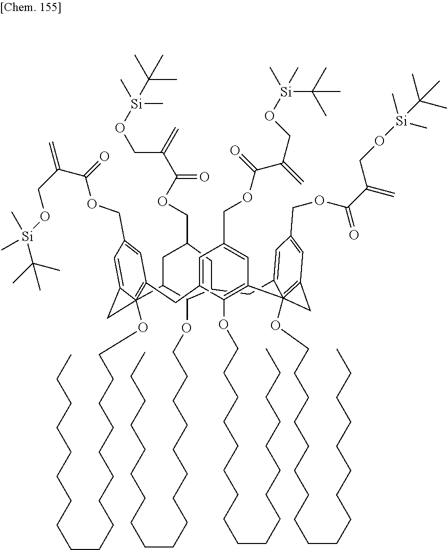

### Synthesis Example 72

A 100 mL four-necked flask equipped with a stirrer, a thermometer, and a reflux condenser tube was charged with 2.50 g (1.49 mmol) of the compound obtained in Synthesis Example 67, 0.538 g (8.96 mmol) of acetic acid, and 60 mL of tetrahydrofuran, and the mixture was stirred. A colorless transparent solution. Then tetrabutylammonium fluoride (an about 1 mol/L tetrahydrofuran solution 8.96 mL (8.96 mmol) was slowly added dropwise to the mixture placed in an ice bath under stirring, and the resulting mixture was further stirred at room temperature for 12 hours. A saturated aqueous ammonium chloride solution was added to the reaction mixture, and then 30 mL of chloroform was added. The reaction mixture was transferred to a separatory funnel, and the organic layer was separated. The aqueous layer was extracted with 30 mL of chloroform twice. The combined organic layer was washed with saturated brine and dried over anhydrous magnesium sulfate. The solvent was removed by evaporation using an evaporator, and a yellow transparent liquid was thereby obtained. The yellow transparent liquid was purified by silica gel column chromatography to thereby obtain a compound represented by the following formula as a white solid (yield amount 1.663 g, yield 91.5%).

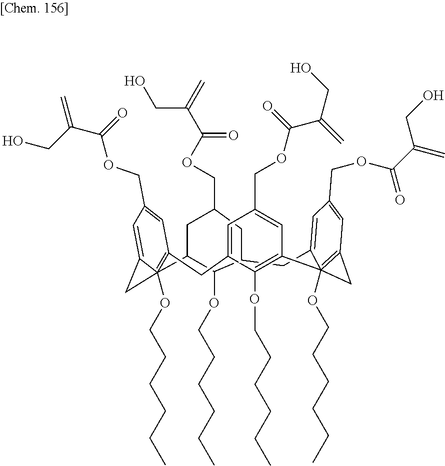

### Synthesis Example 73

The same procedure as in Synthesis Example 72 was repeated except that the compound obtained in Synthesis Example 68 (2.5 g, 1.79 mmol) was used instead of the compound obtained in Synthesis Example 67, to thereby obtain a compound represented by the following formula (1.551 g, yield 92.3%).

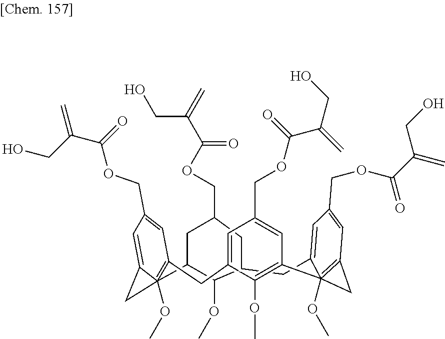

### Synthesis Example 74

The same procedure as in Synthesis Example 72 was repeated except that the compound obtained in Synthesis Example 69 (2.5 g, 1.60 mmol) was used instead of the compound obtained in Synthesis Example 67, to thereby obtain a compound represented by the following formula (1.671 g, yield 94.5%).

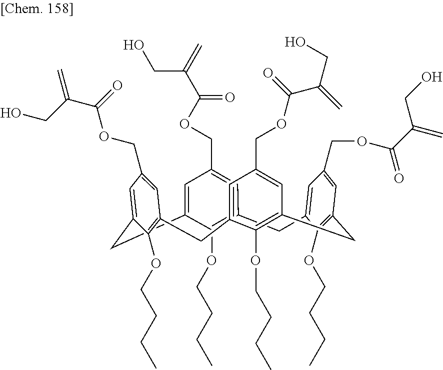 [SMILES: C=C(CO)C(=O)OCc1cc2c(OCCCC)c(c1)Cc1cc(COC(=O)C(=C)CO)cc(c1OCCCC)Cc1cc(COC(=O)C(=C)CO)cc(c1OCCCC)Cc1cc(COC(=O)C(=C)CO)cc(c1OCCCC)C2]

### Synthesis Example 75

The same procedure as in Synthesis Example 72 was repeated except that the compound obtained in Synthesis Example 70 (2.5 g, 1.44 mmol) was used instead of the compound obtained in Synthesis Example 67, to thereby obtain a compound (55-1) represented by the following formula (1.759 g, yield 95.6%).

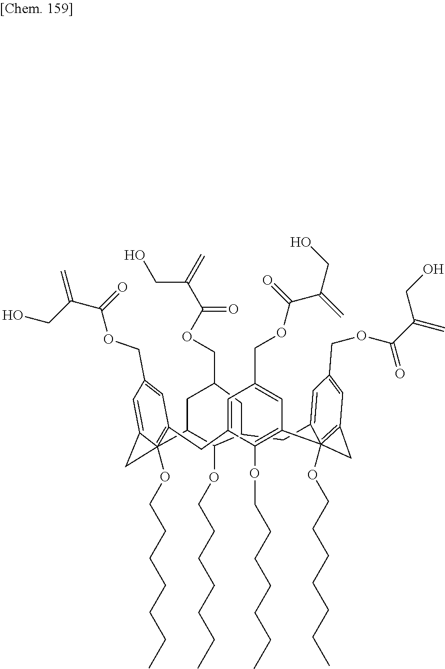

### Synthesis Example 76

The same procedure as in Synthesis Example 72 was repeated except that the compound obtained in Synthesis Example 71 (2.50 g, 1.06 mmol) was used instead of the compound obtained in Synthesis Example 67, to thereby obtain a compound (represented by the following formula (1.90 g, yield 94.8%).

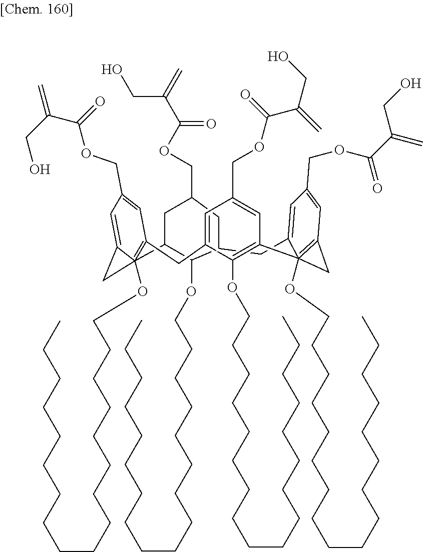

### Example 57

A 100 mL four-necked flask equipped with a stirrer, a thermometer, and a reflux condenser tube was charged with 1.50 g (1.23 mmol) of the compound obtained in Synthesis Example 72, 1.939 g (7.39 mmol) of triphenylphosphine, 0.629 g (7.39 mmol) of acetone cyanohydrin, and 19 mL of tetrahydrofuran, and the mixture was stirred. Then 1.495 g (7.39 mmol) of diisopropyl azodicarboxylate was added dropwise to the mixture placed in an ice bath over 30 minutes, and the mixture was further stirred at room temperature for 48 hours. The reaction solution was concentrated in an evaporator, and hexane was added to precipitate and remove by-products such as triphenylphosphine. The obtained yellow viscous liquid was purified by silica gel column chromatography to thereby obtain the target compound 05-6 (yield amount 0.962 g, yield 62.3%).

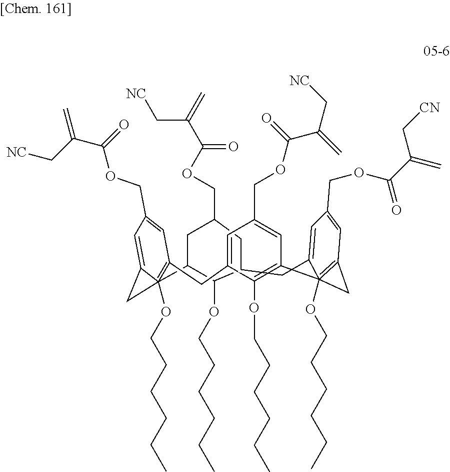

The same procedure as in Example 57 was repeated except that the compound obtained in Synthesis Example 73 (1.50 g, 1.60 mmol) was used instead of the compound obtained in Synthesis Example 72, to thereby obtain the target compound 05-1 (0.784 g, yield 50.3%).

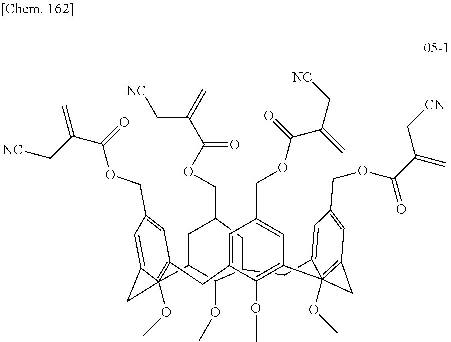

### Example 59

The same procedure as in Example 57 was repeated except that the compound obtained in Synthesis Example 74 (1.50 g, 1.36 mmol) was used instead of the compound obtained in Synthesis Example 72, to thereby obtain the target compound 05-4 (0.861 g, yield 55.6%).

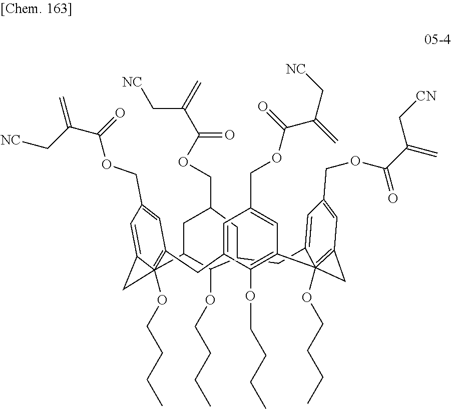

### Example 60

The same procedure as in Example 57 was repeated except that the compound obtained in Synthesis Example 75 (1.50 g, 1.18 mmol) was used instead of the compound obtained in Synthesis Example 72, to thereby obtain the target compound 05-7 (0.984 g, yield 63.8%).

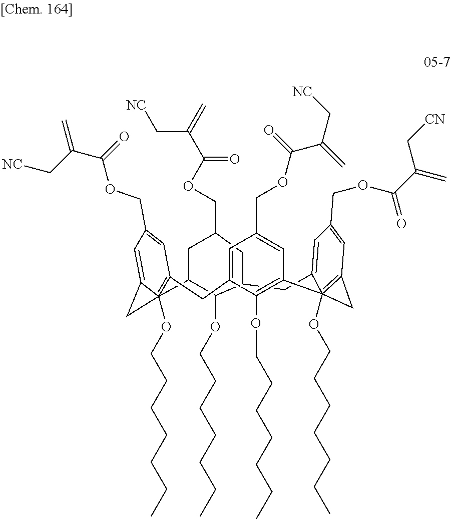

### Example 61

The same procedure as in Example 57 was repeated except that the compound obtained in Synthesis Example 76 (1.5 g, 0.79 mmol) was used instead of the compound obtained in Synthesis Example 72, to thereby obtain the target compound 05-18 (0.940 g, yield 61.5%).

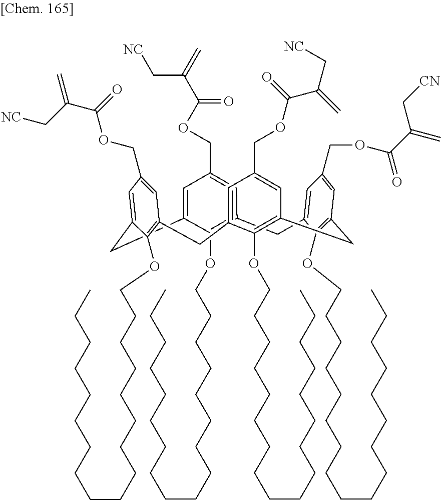 [SMILES: C=C(CC#N)C(=O)OCc1cc2c(OCCCCCCCCCCCCCCCCCC)c(c1)Cc1cc(COC(=O)C(=C)CC#N)cc(c1OCCCCCCCCCCCCCCCCCC)Cc1cc(COC(=O)C(=C)CC#N)cc(c1OCCCCCCCCCCCCCCCCCC)Cc1cc(COC(=O)C(=C)CC#N)cc(c1OCCCCCCCCCCCCCCCCCC)C2]

### Example 62

A 100 mL four-necked flask equipped with a stirrer, a dropping funnel, and a thermometer was charged, in a nitrogen atmosphere, with the compound obtained in Synthesis Example 62 (3.00 g, 3.02 mmol), 2.376 g (9.06 mmol) of triphenylphosphine, 0.771 g (9.06 mmol) of acetone cyanohydrin, and 27 mL of tetrahydrofuran, and the mixture was stirred. Then 1.832 g (9.06 mmol) of diisopropyl azodicarboxylate was added dropwise to the mixture placed in an ice bath over 30 minutes, and the mixture was further stirred at room temperature for 48 hours. The reaction solution was concentrated in an evaporator, and hexane was added to precipitate and remove by-products such as triphenylphosphine. The obtained yellow viscous liquid was used for the next reaction without purification. A 100 mL four-necked flask equipped with a stirrer, a dropping funnel, and a thermometer was charged, in a nitrogen atmosphere, with the crude product obtained above, triethylamine (1.833 g, 18.12 mmol), and methylene chloride (25.3 mL), and the mixture was stirred under cooling with ice. Acrylic acid chloride (1.093 g, 12.08 mmol) was slowly added dropwise. After completion of the dropwise addition, the mixture was stirred at room temperature for 8 hours. Water was added to the reaction mixture, and the mixture was extracted with chloroform (40 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator, and a yellow liquid was thereby obtained. The yellow liquid was purified by silica gel column chromatography to thereby obtain the target compounds 06-6, 07-6, 08-6, and 09-6. 06-6 (0.344 g, yield 10.6%). A mixture of 07-6 and 08-6 (1.606 g, yield 47.5%). 09-6 (0.433 g, yield 12.3%).

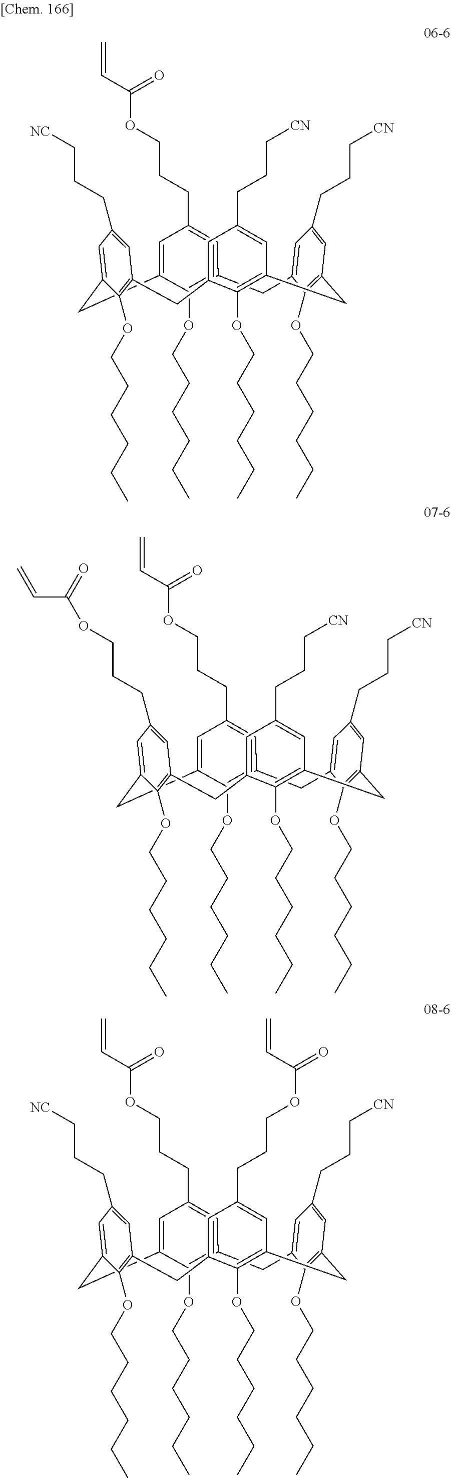

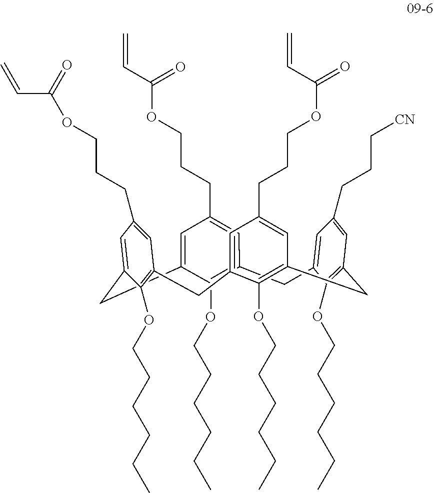 [SMILES: C=CC(=O)OCCCc1cc2c(OCCCCCC)c(c1)Cc1cc(CCCOC(=O)C=C)cc(c1OCCCCCC)Cc1cc(CCCOC(=O)C=C)cc(c1OCCCCCC)Cc1cc(CCCC#N)cc(c1OCCCCCC)C2]

### Example 63

The same procedure as in Example 62 was repeated except that the compound obtained in Synthesis Example 63 (3.00 g, 4.21 mmol) was used instead of the compound obtained in Synthesis Example 62, to thereby obtain the target compounds 06-1, 07-1, 08-1, and 09-1. 06-1 (0.461 g, yield 1 3.8%). A mixture of 07-1 and 08-1 (1.546 g, yield 43.8%). 09-1 (0.391 g, yield 10.5%).

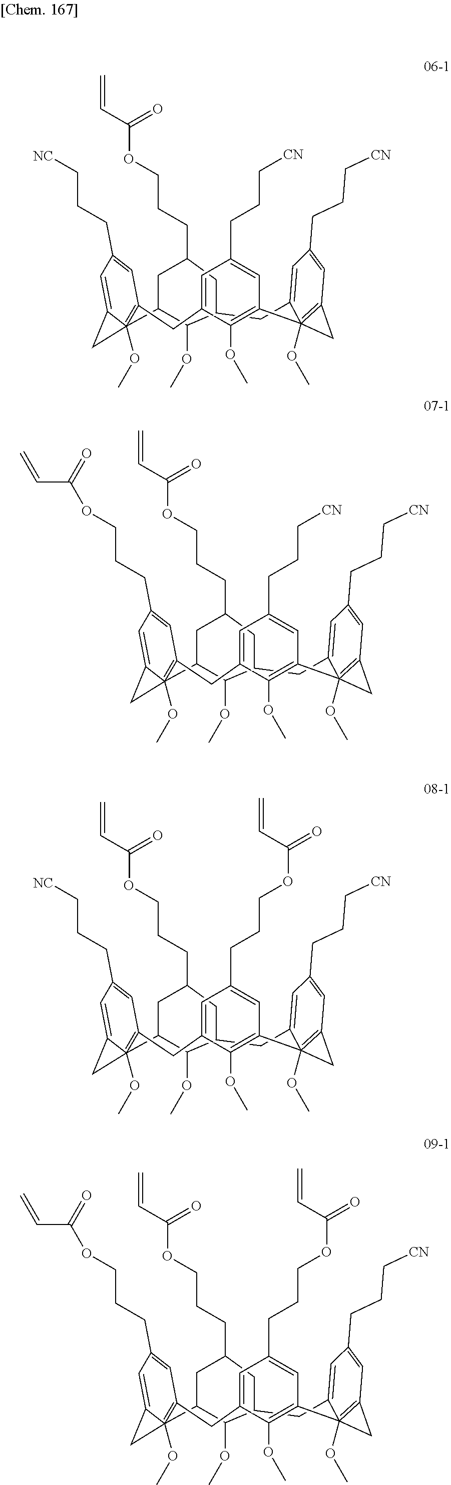

### Example 64

The same procedure as in Example 62 was repeated except that the compound obtained in Synthesis Example 63 (3.00 g, 3.40 mmol) was used instead of the compound obtained in Synthesis Example 62 to thereby obtain the target compounds 06-4, 07-4, 08-4, and 09-4. 06-4 (0.410 g, yield 12.5%). A mixture of 07-1 and 08-1 (1.605 g, yield 46.8%). 09-4 (0.405 g, yield 11.3%).

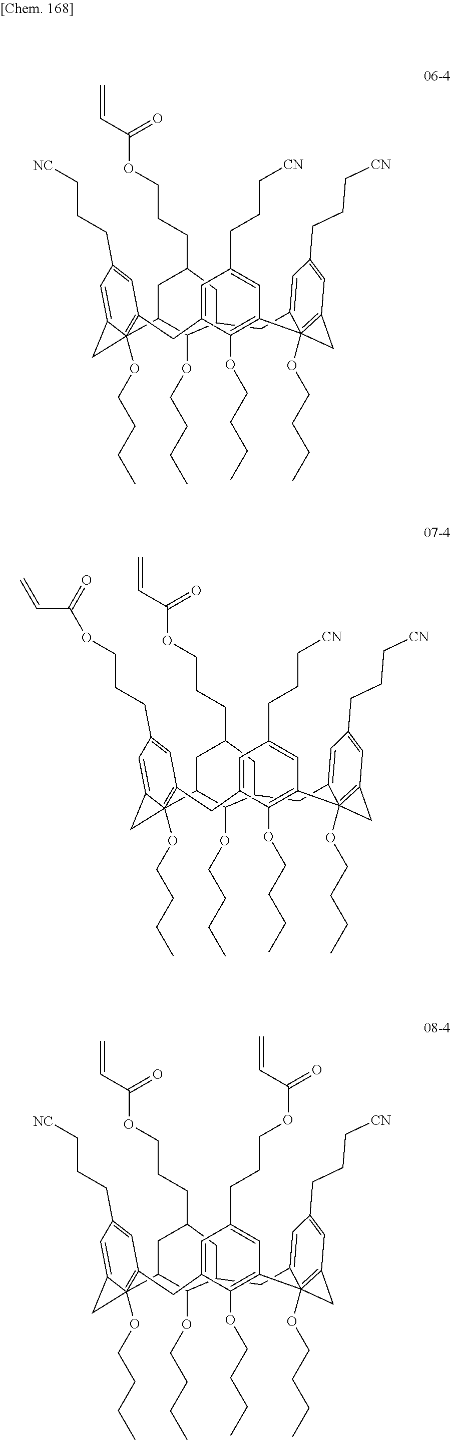

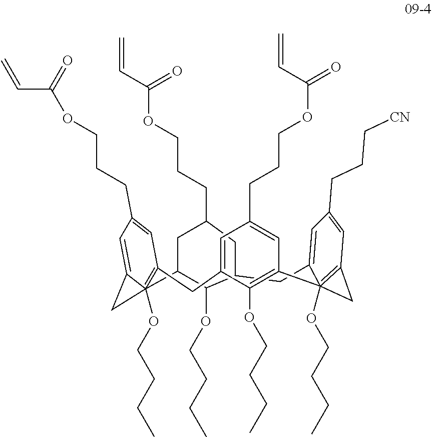

### Example 65

The same procedure as in Example 62 was repeated except that the compound obtained in Synthesis Example 64 (3.00 g, 2.86 mmol) was used instead of the compound obtained in Synthesis Example 62, to thereby obtain the target compounds 06-7, 07-7, 08-7, and 09-7. 06-7 (0.362 g, yield 11.2%). A mixture of 07-7 and 08-7 (1.657 g, yield 49.3%). 09-7 (0.370 g, yield 10.6%).

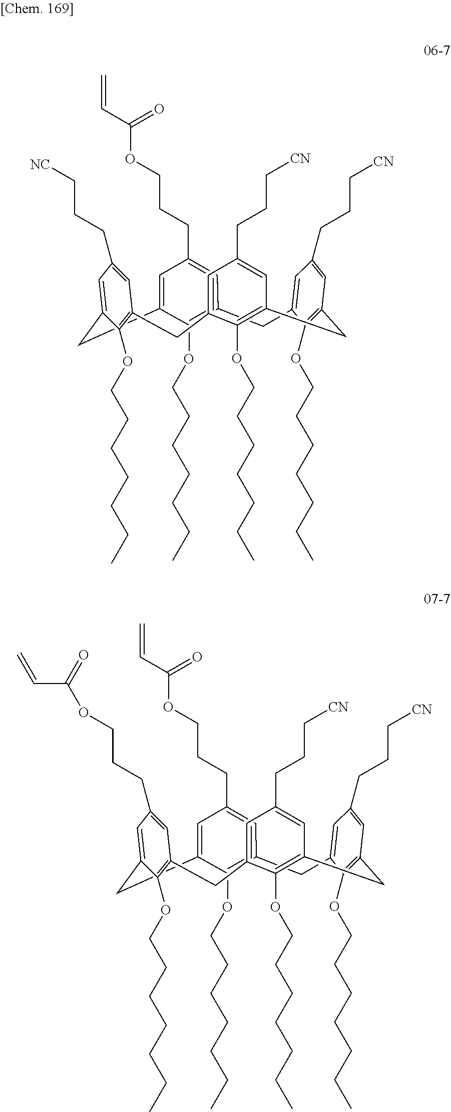 [SMILES: C=CC(=O)OCCCc1cc2c(OCCCCCCC)c(c1)Cc1cc(CCCC#N)cc(c1OCCCCCCC)Cc1cc(CCCC#N)cc(c1OCCCCCCC)Cc1cc(CCCC#N)cc(c1OCCCCCCC)C2 ; C=CC(=O)OCCCc1cc2c(OCCCCCCC)c(c1)Cc1cc(CCCOC(=O)C=C)cc(c1OCCCCCCC)Cc1cc(CCCC#N)cc(c1OCCCCCCC)Cc1cc(CCCC#N)cc(c1OCCCCCCC)C2]

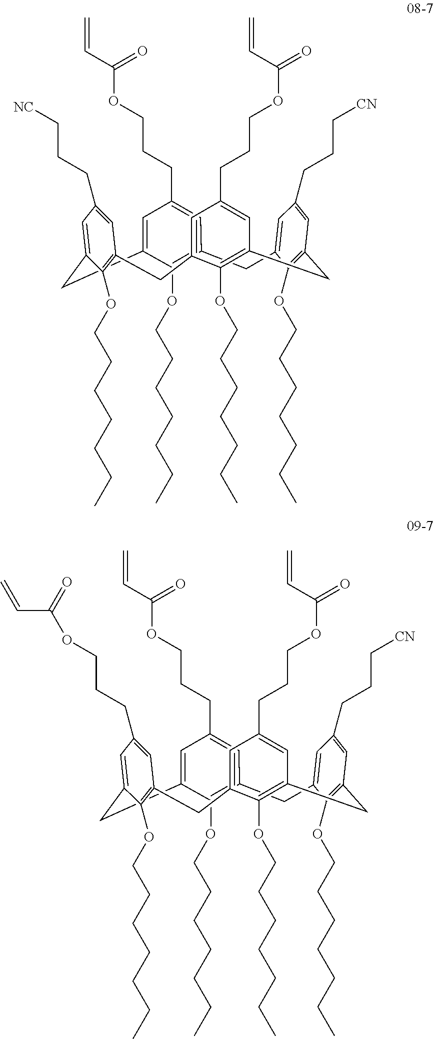 [SMILES: C=CC(=O)OCCCc1cc2c(OCCCCCCC)c(c1)Cc1cc(CCCC#N)cc(c1OCCCCCCC)Cc1cc(CCCOC(=O)C=C)cc(c1OCCCCCCC)Cc1cc(CCCC#N)cc(c1OCCCCCCC)C2 ; C=CC(=O)OCCCc1cc2c(OCCCCCCC)c(c1)Cc1cc(CCCOC(=O)C=C)cc(c1OCCCCCCC)Cc1cc(CCCOC(=O)C=C)cc(c1OCCCCCCC)Cc1cc(CCCC#N)cc(c1OCCCCCCC)C2]

### Example 66

The same procedure as in Example 62 was repeated except that the compound obtained in Synthesis Example 65 (3.00 g, 1.80 mmol) was used instead of the compound obtained in Synthesis Example 62, to thereby obtain the target compounds 06-18, 07-18, 08-18, 09-18. 06-18 (0.308 g, yield 9.8%). A mixture of 07-18 and 08-18 (1.413 g, yield 43.8%). 09-18 (0.400 g, yield 12.1%).

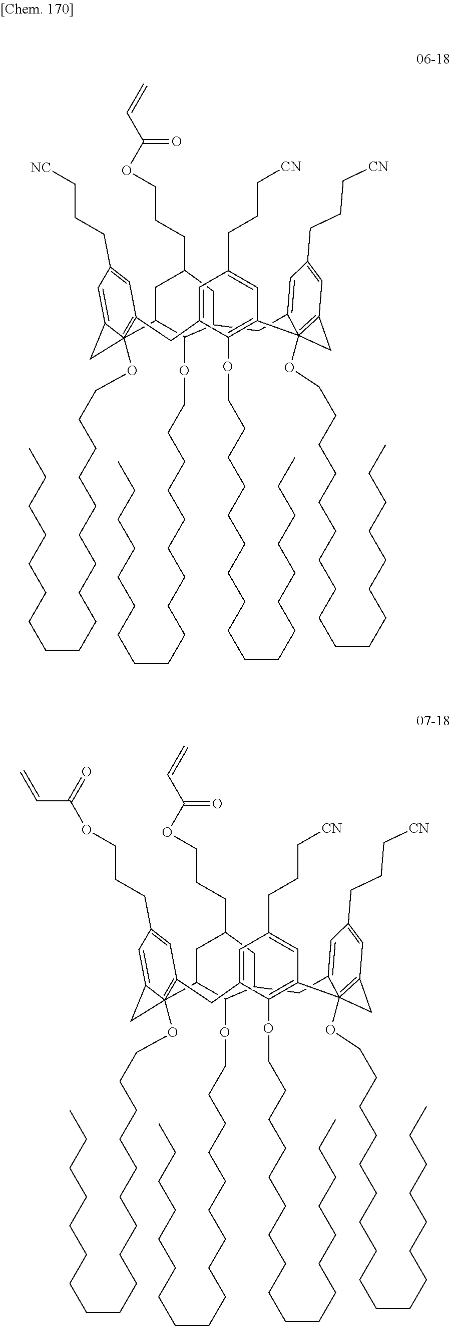

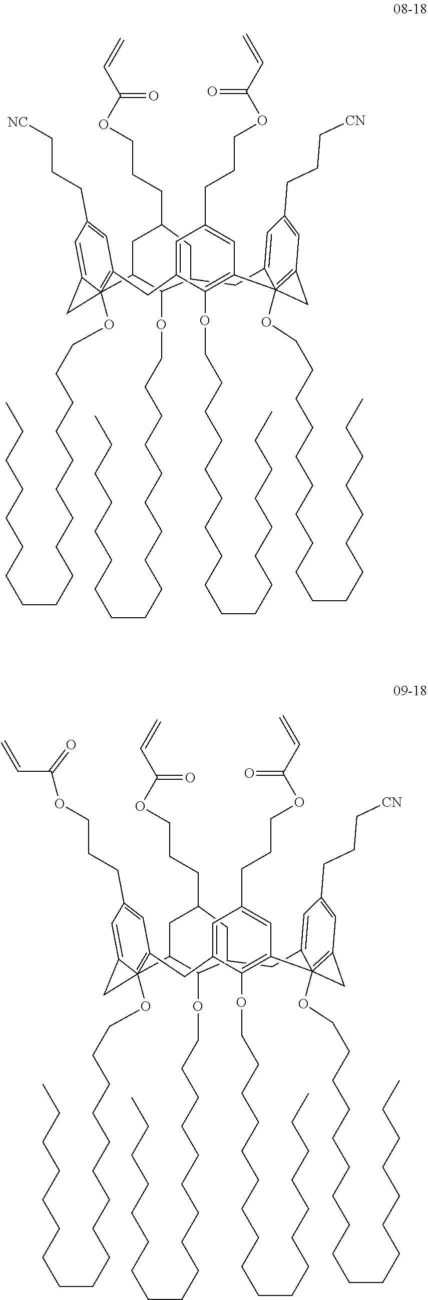

### Synthesis Example 77

A 100 mL four-necked flask equipped with a stirrer, a thermometer, and a reflux condenser tube was charged with 2.50 g (2.52 mmol) of the compound obtained in Synthesis Example 62, 3.96 g (15.10 mmol) of triphenylphosphine, 3.267 g (15.10 mmol) of 2-[[[(1,1-dimethylethyl)dimethylsilyl]oxy]2-propenoic acid, and 43 mL of tetrahydrofuran, and the mixture was stirred. Then 3.053 g (15.10 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography, to thereby obtain a compound represented by the following formula as a light yellow solid (3.251 g, yield 72.3%).

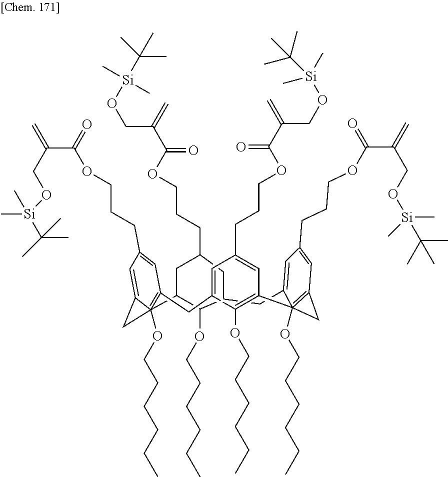

### Synthesis Example 78

The same procedure as in Synthesis Example 77 was repeated except that the compound obtained in Synthesis Example 63 (2.50 g, 3.33 mmol) was used instead of the compound obtained in Synthesis Example 62, to thereby obtain a compound represented by the following formula (3.782 g, yield 71.6%).

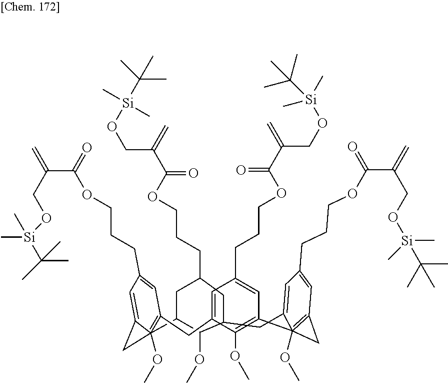

### Synthesis Example 79

The same procedure as in Synthesis Example 77 was repeated except that the compound obtained in Synthesis Example 64 (2.50 g, 2.84 mmol) was used instead of the compound obtained in Synthesis Example 62, to thereby obtain a compound represented by the following formula (3.553 g, yield 74.8%).

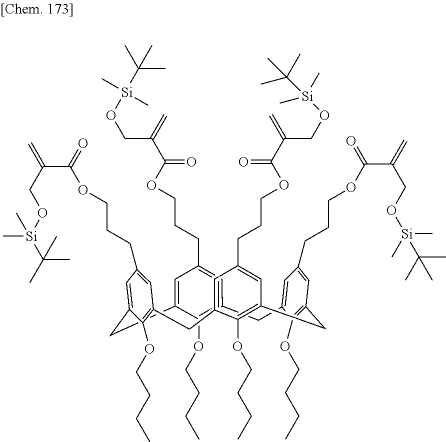 [SMILES: C=C(CO[Si](C)(C)C(C)(C)C)C(=O)OCCCc1cc2c(OCCCC)c(c1)Cc1cc(CCCOC(=O)C(=C)CO[Si](C)(C)C(C)(C)C)cc(c1OCCCC)Cc1cc(CCCOC(=O)C(=C)CO[Si](C)(C)C(C)(C)C)cc(c1OCCCC)Cc1cc(CCCOC(=O)C(=C)CO[Si](C)(C)C(C)(C)C)cc(c1OCCCC)C2]

### Synthesis Example 80

The same procedure as in Synthesis Example 77 was repeated except that the compound obtained in Synthesis Example 65 (2.50 g, 2.38 mmol) was used instead of the compound obtained in Synthesis Example 62, to thereby obtain a compound represented by the following formula (3.305 g, yield 75.3%).

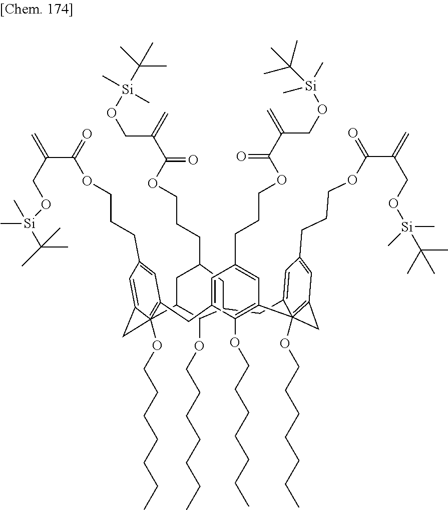

### Synthesis Example 81

The same procedure as in Synthesis Example 77 was repeated except that the compound obtained in Synthesis Example 66 (2.50 g, 1.50 mmol) was used instead of the compound obtained in Synthesis Example 62, to thereby obtain a compound represented by the following formula (3.011 g, yield 81.6%).

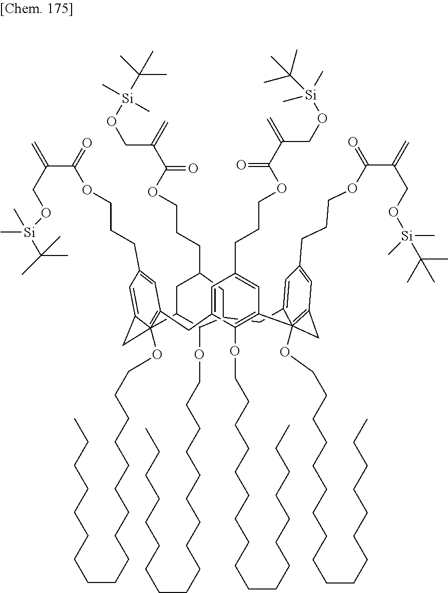

### Synthesis Example 82

A 200 mL four-necked flask equipped with a stirrer, a thermometer, and a reflux condenser tube was charged with 3.50 g (1.96 mmol) of the compound obtained in Synthesis Example 77, 0.706 g (11.75 mmol) of acetic acid, and 78.4 mL of tetrahydrofuran, and the mixture was stirred. A colorless transparent solution. Then tetrabutylammonium fluoride (an about 1 mol/L tetrahydrofuran solution 11.75 mL (11.75 mmol) was slowly added dropwise to the mixture placed in an ice bath under stirring. The mixture was stirred at room temperature for 12 hours. A saturated aqueous ammonium chloride solution was added to the reaction mixture, and then 50 mL of chloroform was added. The reaction mixture was transferred to a separatory funnel, and the organic layer was separated. Then the aqueous layer was extracted with 50 mL of chloroform twice. The combined organic layer was washed with saturated brine and dried over anhydrous magnesium sulfate. The solvent was removed by evaporation using an evaporator, and a yellow transparent liquid was thereby obtained. The yellow transparent liquid was purified by silica gel column chromatography to thereby obtain a compound represented by the following formula (yield amount 2.417 g, yield 92.8%).

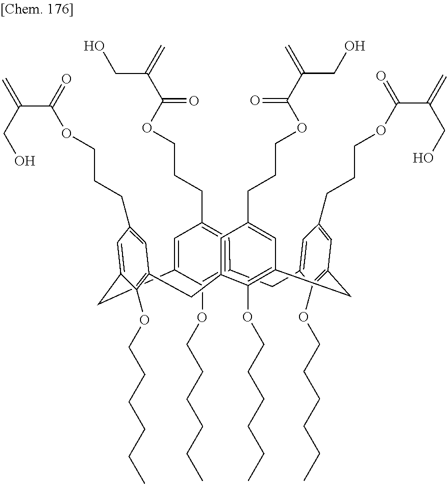 [SMILES: C=C(CO)C(=O)OCCCc1cc2c(OCCCCCC)c(c1)Cc1cc(CCCOC(=O)C(=C)CO)cc(c1OCCCCCC)Cc1cc(CCCOC(=O)C(=C)CO)cc(c1OCCCCCC)Cc1cc(CCCOC(=O)C(=C)CO)cc(c1OCCCCCC)C2]

### Synthesis Example 83

The same procedure as in Synthesis Example 82 was repeated except that the compound obtained in Synthesis Example 78 (3.50 g, 2.32 mmol) was used instead of the compound obtained in Synthesis Example 77, to thereby obtain a compound represented by the following formula (2.214 g, yield 90.8%).

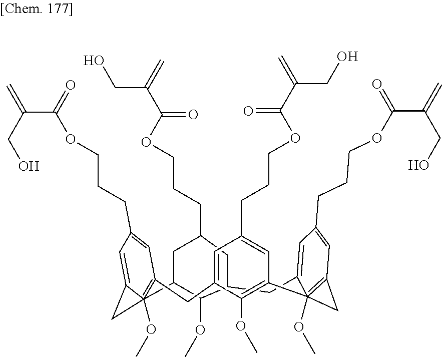

### Synthesis Example 84

The same procedure as in Synthesis Example 82 was repeated except that the compound obtained in Synthesis Example 79 (3.50 g, 2.32 mmol) was used instead of the compound obtained in Synthesis Example 77, to thereby obtain a compound represented by the following formula (2.344 g, yield 92.1%).

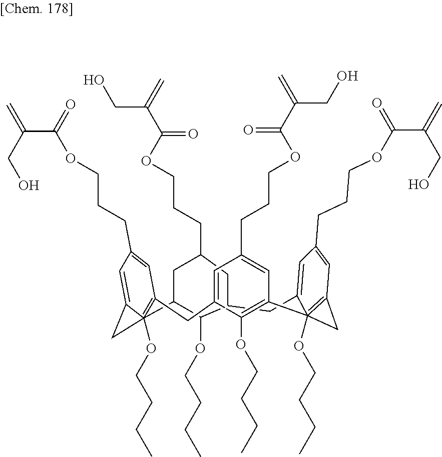

### Synthesis Example 85

The same procedure as in Synthesis Example 82 was repeated except that the compound obtained in Synthesis Example 80 (3.50 g, 2.32 mmol) was used instead of the compound obtained in Synthesis Example 77, to thereby obtain a compound represented by the following formula (2.466 g, yield 93.7%).

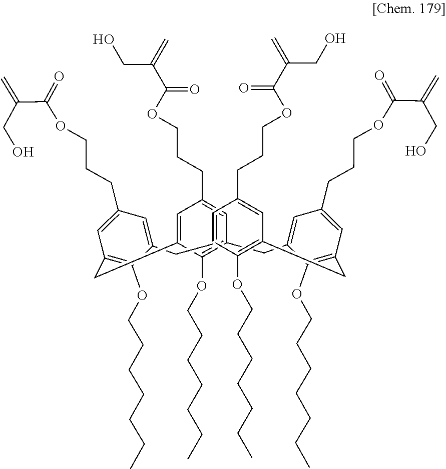

### Synthesis Example 86

The same procedure as in Synthesis Example 82 was repeated except that the compound obtained in Synthesis Example 81 (3.50 g, 1.42 mmol) was used instead of the compound obtained in Synthesis Example 77, to thereby obtain a compound represented by the following formula (2.608 g, yield 91.5%).

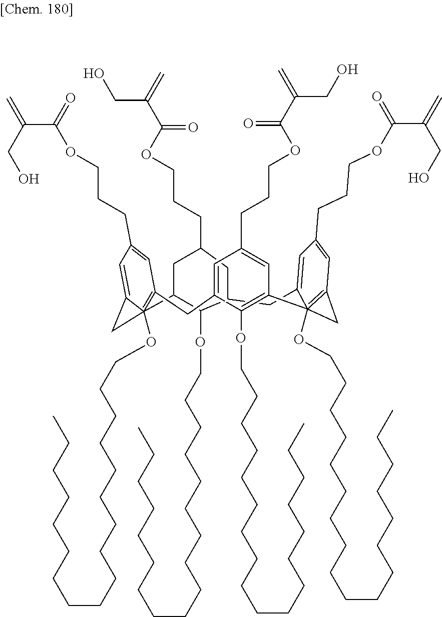

### Example 67

A 100 mL four-necked flask equipped with a stirrer, a thermometer, and a reflux condenser tube was charged with 2.00 g (1.50 mmol) of the compound obtained in Synthesis Example 82, 2.367 g (9.00 mmol) of triphenylphosphine, 0.768 g (9.00 mmol) of acetone cyanohydrin, and 24 mL of tetrahydrofuran, and the mixture was stirred. Then 1.825 g (9.00 mmol) of diisopropyl azodicarboxylate was added dropwise to the mixture placed in an ice bath over 30 minutes, and the mixture was further stirred at room temperature for 48 hours. The reaction solution was concentrated in an evaporator, and hexane was added to precipitate and remove by-products such as triphenylphosphine. The obtained yellow viscous liquid was purified by silica gel column chromatography to thereby obtain the target compound 010-6 (yield amount 1.28 g, yield 62.3%).

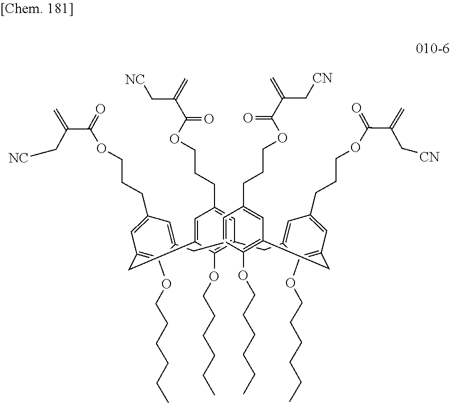

### Example 68

The same procedure as in Example 67 was repeated except that the compound obtained in Synthesis Example 83 (2.00 g, 1.91 mmol) was used instead of the compound obtained in Synthesis Example 82, to thereby obtain the target compound 010-1 (1.065 g, yield 51.5%).

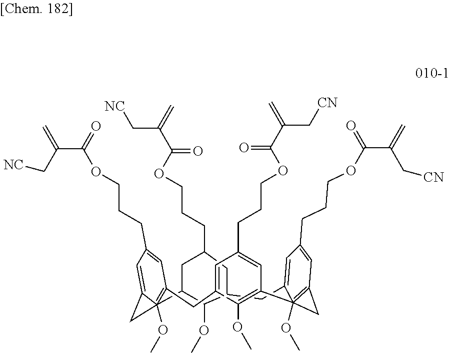

### Example 69

The same procedure as in Example 67 was repeated except that the compound obtained in Synthesis Example 84 (2.00 g, 1.64 mmol) was used instead of the compound obtained in Synthesis Example 82, to thereby obtain the target compound 010-4 (1.182 g, yield 57.4%).

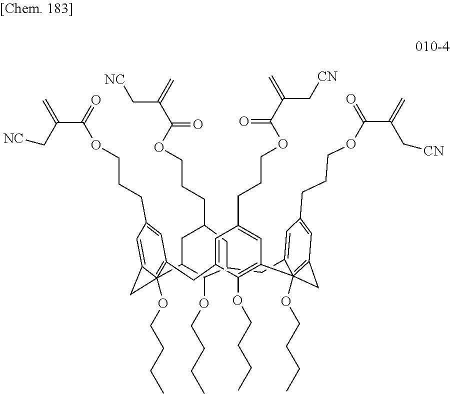

### Example 70

The same procedure as in Synthesis Example 67 was repeated except that the compound obtained in Synthesis Example 85 (2.00 g, 1.44 mmol) was used instead of the compound obtained in Synthesis Example 82, to thereby obtain the target compound 010-7 (1.248 g, yield 60.8%).

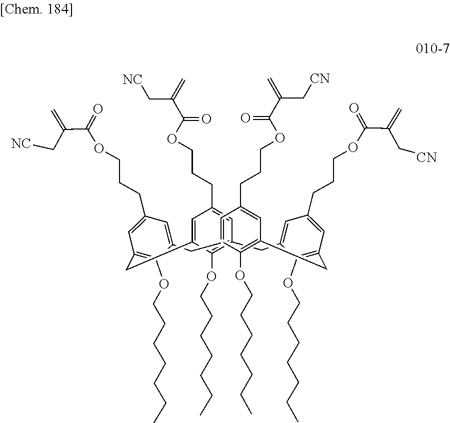

The same procedure as in Synthesis Example 67 was repeated except that the compound obtained in Synthesis Example 86 (2.00 g, 1.00 mmol) was used instead of the compound obtained in Synthesis Example 82, to thereby obtain the target compound 010-18 (1.189 g, yield 58.4%).

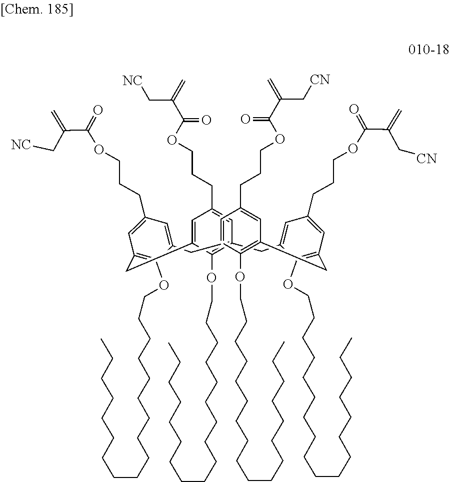

### Comparative Example

A 100 mL four-necked flask equipped with a stirrer, a thermometer, and a reflux condenser tube was charged with 1.00 g (1.212 mmol) of the compound obtained in Synthesis Example 20, 10.00 g (138.7 mmol) of tetrahydrofuran, 1.907 g (7.271 mmol) of triphenylphosphine, and 0.6260 g (7.271 mmol) of methacrylic acid, and the mixture was stirred. A light yellow transparent solution. Then 1.470 g (7.271 mmol) of diisopropyl azodicarboxylate was added dropwise to the mixture placed in an ice bath over 30 minutes. A light yellow transparent solution. The solution was stirred at room temperature for 6 hours. Hexane was added to the reaction solution to precipitate and remove by-products such as triphenyiphosphine, and the resulting solution was extracted with chloroform, washed with water and saturated brine, and dried over magnesium sulfate. The solvent was removed by evaporation using an evaporator, and the resulting orange viscus liquid was subjected to column chromatography (eluent: n-hexane:acetone=90:10) to thereby obtain compound (1′) represented by the following formula. Compound (1′) was vacuum dried (at 60° C. for 6 hours or longer). 0.9058 g, and the yield was 68.1%.

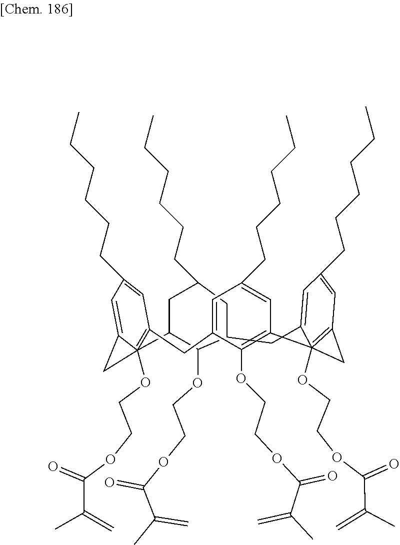

<Production of Curable Compositions>

0.25 g of one of the obtained calixarene compounds, 0.25 g of dipentaerythritol hexaacrylate (“A-DPH” manufactured by Shin Nakamura Chemical Co., Ltd.), 0.005 g of a polymerization initiator (“Irgacure 369” manufactured by BASF), and 9.5 g of propylene glycol monomethyl ether acetate were mixed to obtain a curable composition.

<Production of Layered Bodies>

The curable composition was applied to substrates 1 to 4 below by a spin coating method such that the thickness of the coating after curing was about 0.5 μm and then dried on a hot plate at 100° C. for 2 minutes. A high-pressure mercury lamp was used to irradiate the curable composition with UV rays at 500 mJ/cm² in a nitrogen atmosphere to cure the curable composition, and layered bodies were thereby obtained.

Substrate 1: polymethyl methacrylate resin plate

Substrate 2: aluminum plate

Substrate 3: polyethylene terephthalate film having a SiO₂ thin layer (thickness 100 nm) (the curable composition was applied to the SiO₂ thin film)

<Evaluation of Adhesion>

A layered body stored in a 23° C. and 50% RH environment for 24 hours was used, and the adhesion was evaluated according to JIS K6500-5-6 (adhesive strength: a cross-cut method). A cellophane tape used was “CT-24” manufactured by Nichiban Co., Ltd. The criteria for the evaluation are as follows.

A: 80 or more out of 100 squares were not detached and remained present.

B: 50 to 79 out of 100 squares were not detached and remained present.

C: The number of squares that were not detached and remained present was 49 or less out of 100 squares.

<Evaluation of Moist Heat Resistance>

One of the curable compositions was applied to a 5 inch SiO substrate to a film thickness of about 50 μm using an applicator and dried on a hot plate at 100° C. for 2 minutes. A mask having an LS pattern with L/S=50 μm/50 μm was brought into tight contact with the coating obtained, and a high-pressure mercury lamp was used to irradiate the composition with UV rays at 1000 mJ/cm² in a nitrogen atmosphere to cure the composition. The substrate exposed to the light was developed using ethyl acetate to obtain an evaluation substrate. The obtained substrate was stored in a thermo-hygrostat at 85° C. and 85% RH for 100 hours, and a laser microscope (“VK-X200” manufactured by KEYENCE CORPORATION) was used to check the state of the pattern after a lapse of 100 hours. The criteria for the evaluation are as follows.

A: The entire pattern was well modified and maintained.

B: Cracking or chipping was observed in part of the pattern.

C: Cracking or chipping was observed in the pattern, and delamination of the pattern was also observed.

TABLE 1

| Calixarene compound | | 1-6 | 1-4 | 1-7 | 2-6 | 2-4 | 2-7 |
|---|---|---|---|---|---|---|---|
| Adhesion | Substrate 1 | A | A | A | A | A | A |
| | Substrate 2 | A | A | A | A | A | A |
| | Substrate 3 | A | A | A | A | A | A |
| Moist heat resistance | | A | A | A | A | A | A |
| Calixarene compound | | 3-6 | 4-6 | 5-6 | 6-6 | 7-6 | 8-6 |
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 9-6 | 10-6 | 11-6 | 12-6 | 13-6 | 14-6 |
|---|---|---|---|---|---|---|---|
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 2

| Calixarene compound | | 15-6 | 16-6 | 17-6 | 17-4 | 17-7 | 17-18 |
|---|---|---|---|---|---|---|---|
| Adhesion | Substrate 1 | A | A | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 17-1 | 18-6 | 18-4 | 18-7 | 18-18 | 18-1 |
| Adhesion | Substrate 1 | A | A | A | A | B | A |
| | Substrate 2 | A | A | A | A | A | A |
| | Substrate 3 | A | A | A | A | A | A |
| Moist heat resistance | | A | A | A | A | A | B |
| Calixarene compound | | 19-6 | 20-6 | 21-6 | 22-6 | 23-6 | 24-6 |
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 3

| Calixarene compound | | 25-6 | 26-6 | 27-6 | 28-6 | 29-6 | 30-6 |
|---|---|---|---|---|---|---|---|
| Adhesion | Substrate 1 | A | A | A | A | A | A |
| | Substrate 2 | A | A | A | A | A | A |
| | Substrate 3 | A | A | A | A | A | A |
| Moist heat resistance | | A | A | A | A | A | A |
| Calixarene compound | | 31-6 | 32-18 | 33-6 | 33-4 | 33-7 | 33-18 |
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 33-1 | 34-6 | 35-6 | 01-6 | 02&03-6 | 04-6 |
|---|---|---|---|---|---|---|---|
| Adhesion | Substrate 1 | A | A | A | A | A | B |
| | Substrate 2 | A | A | B | A | A | A |
| | Substrate 3 | A | A | A | A | A | A |

| | | | | | | |
|---|---|---|---|---|---|---|
| Moist heat resistance | A | A | A | A | A | A |

TABLE 4

| Calixarene compound | | 01-1 | 02&03-1 | 04-1 | 01-4 | 02&03-4 | 04-4 |
|---|---|---|---|---|---|---|---|
| Adhesion | Substrate 1 | A | A | B | A | A | 8 |
| | Substrate 2 | A | A | A | A | A | A |
| | Substrate 3 | A | A | A | A | A | A |
| Moist heat resistance | | A | A | A | A | A | A |
| Calixarene compound | | 01-7 | 02&03-7 | 04-7 | 01-18 | 02&03-18 | 04-18 |
| Adhesion | Substrate 1 | A | A | B | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 05-6 | 05-1 | 05-4 | 05-7 | 05-18 | 06-6 |
|---|---|---|---|---|---|---|---|
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 5

| Calixarene compound | | 07&08-6 | 09-6 | 06-1 | 07&08-1 | 09-1 | 06-4 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |
| Moist heat resistance | | A | A | A | A | A | A |
| Calixarene compound | | 07&08-4 | 09-4 | 06-7 | 07&08-7 | 09-7 | 06-18 |
| Adhesion | Substrate 1 | A | 8 | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 07&08-18 | 09-18 | 010-6 | 010-1 | 010-4 | 010-7 |
|---|---|---|---|---|---|---|---|
| Adhesion | Substrate 1 | A | B | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 6

| | | |
|---|---|---|
| Calixarene compound | 010-18 | 1′ |

| | | | |
|---|---|---|---|
| Adhesion | Substrate 1 | A | C |
| | Substrate 2 | A | C |
| | Substrate 3 | A | C |

| | | |
|---|---|---|
| Moist heat resistance | A | B |

### Example Group <II>

### Synthesis Example 1

A 20 L separable four-necked flask equipped with a stirrer, a thermometer, and a reflux condenser tube was quickly charged with 1000 g (1.54 mol) of t-butyl-calix[4]arene, 1159 g (12.32 mol) of phenol, and 9375 mL of dehydrated toluene, and the mixture was stirred at 300 rpm under nitrogen flow. The t-butyl-calix[4]arene used as a raw material was not dissolved but was suspended. Then, while the flask was cooled in an ice bath, 1643 g (12.32 mol) of anhydrous aluminum chloride (III) was added in several portions. The solution turned into a light orange transparent solution, and the anhydrous aluminum chloride (III) precipitated on the bottom. The mixture was allowed to react at room temperature for 5 hours. Then the contents were transferred to a 1 L beaker, and 20 kg of ice, 10 L of 1N hydrochloric acid, and 20 L of chloroform were added to stop the reaction. The mixture turned into a light yellow transparent solution. This reaction mixture was transferred to a separatory funnel, and the organic layer was separated. Then the aqueous layer was extracted with 5 L of chloroform three times, and the extract was combined with the organic layer. The organic layer was pre-dried over anhydrous magnesium sulfate and filtered. The solvent was removed by evaporation using an evaporator, and a mixture of white crystals and a colorless transparent solution was thereby obtained. Methanol was added slowly to the mixture under stirring to reprecipitate the crystals. The white crystals were filtered on a Kiriyama funnel and washed with methanol. The obtained white crystals were vacuum dried (at 50° C. for 6 hours or longer) to thereby obtain 597 g of the target intermediate A. The yield was 91%.

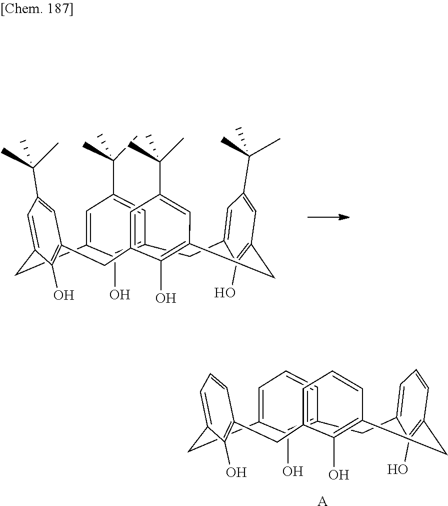

### Synthesis Example 2

A 2 L four-necked flask equipped with a stirrer, a thermometer, and a reflux condenser tube was charged with 205 g (1.52 mol) of n-hexanoyl chloride and 709 g of nitroethane, and the mixture was stirred. Then, while the flask was cooled in an ice bath, 243 g (1.82 mol) of anhydrous aluminum chloride (III) was added in several portions. The solution turned into a light orange transparent solution. The resulting solution was stirred at room temperature for 30 minutes, and 100 g (0.236 mol) of intermediate A was added in several portions. The mixture foamed and turned into an orange transparent solution. The solution was allowed to react at room temperature for 5 hours. Then the contents were slowly transferred to a 2 L beaker containing 450 mL of chloroform and 956 g of ice water to stop the reaction. Then 1N hydrochloric acid was added until the pH was 1. The reaction mixture was transferred to a separatory funnel, and the organic layer was separated. Next, the aqueous layer was extracted with 400 mL of chloroform three times, and the extract was combined with the organic layer. The organic layer was pre-dried over anhydrous magnesium sulfate. The solvent was removed by evaporation using an evaporator, and a yellow transparent solution was thereby obtained. While the solution was cooled in an ice bath, methanol was added to reprecipitate crystals. The white crystals were filtered on a Kiriyama funnel and recrystallized with chloroform and methanol. The obtained white crystals were vacuum dried (at 60° C. for 6 hours or longer) to thereby obtain 122 g of compound B-6 represented by the following structural formula. The yield was 63%.

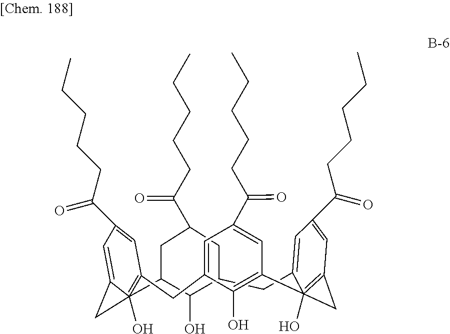

### Synthesis Example 3

The same procedure as in Synthesis Example 2 was repeated except that butyl chloride was used instead of n-hexanoyl chloride, to thereby obtain 106 g of compound B-4 represented by the following structural formula. The yield was 64%.

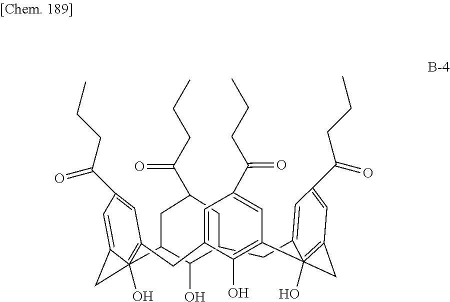

### Synthesis Example 4

The same procedure as in Synthesis Example 2 was repeated except that n-heptanoyl chloride was used instead of n-hexanoyl chloride, to thereby obtain 134 g of compound B-7 represented by the following structural formula. The yield was 65%.

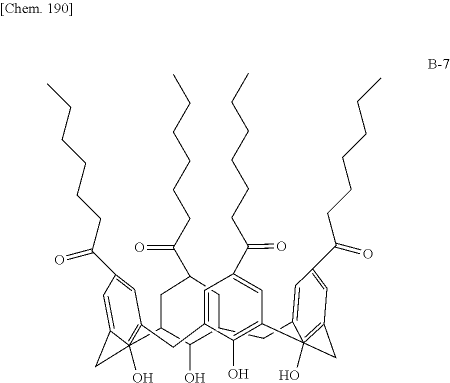

### Synthesis Example 5

The same procedure as in Synthesis Example 2 was repeated except that stearoyl chloride was used instead of n-hexanoyl chloride, to thereby obtain 228 g of compound B-18 represented by the following structural formula. The yield was 65%.

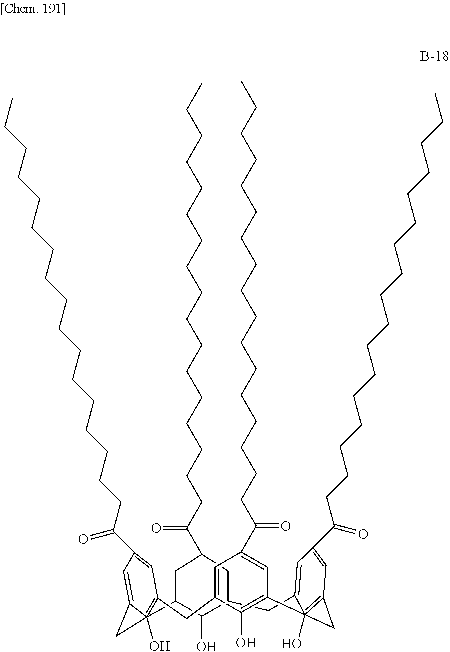

### Synthesis Example 6

A 100 mL four-necked flask equipped with a stirrer, a thermometer, and a reflux condenser tube was charged with 5.00 g (6.119 mmol) of B-6, 17.0 g of acetonitrile, 11.28 g (48.95 mmol) of potassium carbonate, 0.813 g (4.896 mmol) of potassium iodide, and 7.489 g (48.95 mmol) of methyl 2-bromoacetate, and the mixture was allowed to react at 70° C. for 24 hours. The resulting mixture was cooled to room temperature, and ion exchanged water and 0.3N hydrochloric acid were added until the pH was 6. 50 g of chloroform was added. The reaction mixture was transferred to a separatory funnel, and the organic layer was separated. Next, the aqueous layer was extracted with 50 g of chloroform three times, and the extract was combined with the organic layer. The organic layer was pre-dried over anhydrous magnesium sulfate. The solvent was removed by evaporation using an evaporator, and a red waxy solid was thereby obtained. The obtained red waxy solid was vacuum dried (at 60° C. for 6 hours or longer) to thereby obtain 5.04 g of compound C-6 represented by the following structural formula. The yield was 74.5%.

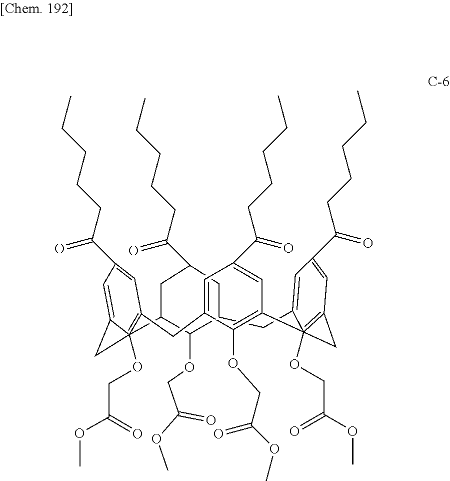

### Synthesis Example 7

The same procedure as in Synthesis Example 6 was repeated except that B-4 was used instead of B-6, to thereby obtain 4.88 g of compound C-4 represented by the following structural formula with a yield of 69.3%.

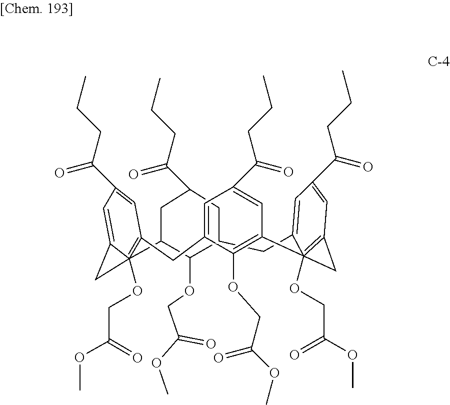

### Synthesis Example 8

The same procedure as in Synthesis Example 6 was repeated except that B-7 was used instead of B-6, to thereby obtain 5.12 g of compound C-7 represented by the following structural formula with a yield of 77.0%.

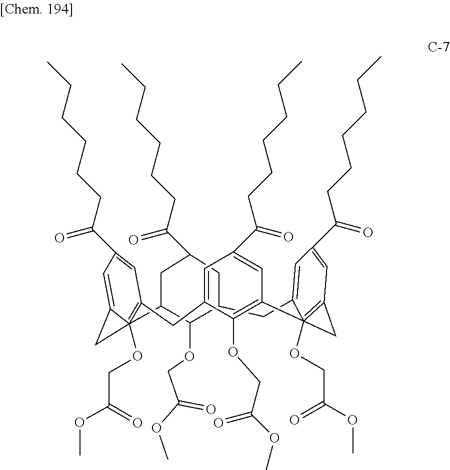

### Synthesis Example 9

The same procedure as in Synthesis Example 6 was repeated except that B-18 was used instead of B-6, to thereby obtain 5.34 g of compound C-18 represented by the following structural formula with a yield of 89.5%.

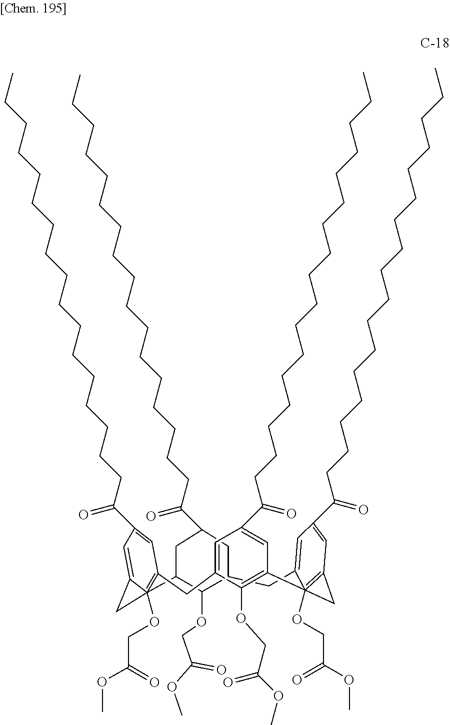

### Synthesis Example 10

A 500 mL four-necked flask equipped with a stirrer, a thermometer, and a reflux condenser tube was charged with 16.44 g of tetrahydrofuran in an ice bath, and 1.038 g (27.35 mmol) of lithium aluminum hydride was slowly added. 5.04 g (4.559 mmol) of C-6 diluted with 49.31 g of tetrahydrofuran was added dropwise from a dropping funnel such that the temperature did not exceed 10° C. The resulting gray suspension reaction solution was allowed to react at room temperature for 6 hours. 30 g of chloroform was added to the resulting solution placed in an ice bath, and 30 g of 5N hydrochloric acid was added dropwise to stop the reaction. Then diatomaceous earth was used to filter the reaction solution, and the filtrate was transferred to a separatory funnel to separate the organic layer. Next, the aqueous layer was extracted with 30 g of chloroform three times, and the extract was combined with the organic layer. The organic layer was pre-dried over anhydrous magnesium sulfate. The solvent was removed by evaporation using an evaporator, and a light yellow liquid was thereby obtained. By-products were removed by column chromatography using an eluent: n-hexane:ethyl acetate=1:1, and then the target compound was eluted with an eluent: chloroform:isopropyl alcohol=5:1. The eluent was removed by evaporation under reduced pressure to thereby obtain 2.857 g of white solid compound D-6 represented by the following structural formula. The yield was 63.1%.

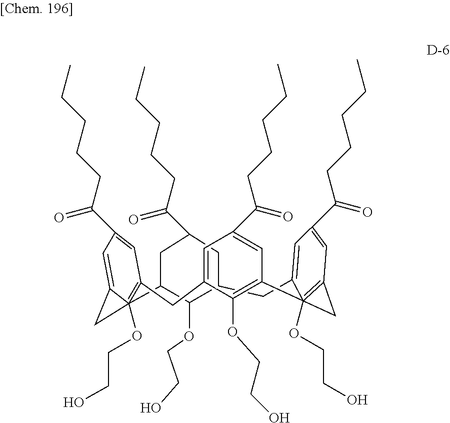

### Synthesis Example 11

The same procedure as in Synthesis Example 10 was repeated except that C-4 was used instead of C-6, to thereby obtain 3.06 g of compound D-4 represented by the following structural formula with a yield of 69.0%.

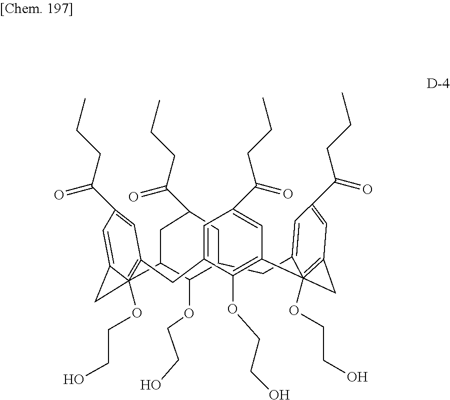

### Synthesis Example 12

The same procedure as in Synthesis Example 10 was repeated except that C-7 was used instead of C-6, to thereby obtain 3.11 g of compound D-7 represented by the following structural formula with a yield of 68.2%.

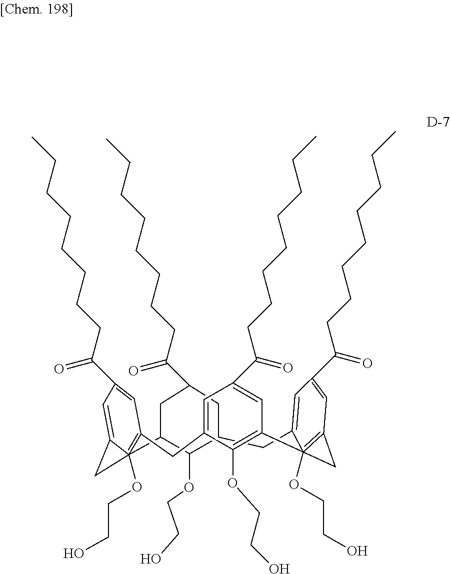

### Example 1

A 50 mL four-necked flask equipped with a stirrer, a thermometer, and a reflux condenser tube was charged with 1.00 g (1.007 mmol) of D-6, 2.904 g of tetrahydrofuran, 2.112 g (8.054 mmol) of triphenylphosphine, 0.173 g (2.014 mmol) of methacrylic acid, and 0.786 g (6.041 mmol) of monomethyl maleate, and the mixture was stirred. An ocher suspension solution was thereby obtained. Then 1.810 g (8.054 mmol) of diisopropyl azodicarboxylate diluted with 1.452 g of tetrahydrofuran was added dropwise to the suspension solution placed in an ice bath over 30 minutes. The resulting orange transparent reaction solution was stirred at room temperature for 10 hours. Hexane was added to the reaction solution to precipitate and remove by-products such as triphenylphosphine, and then the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:ethyl acetate=85:15) to thereby obtain 0.402 g of the target compound 1-6 with a yield of 28.6%, 0.181 g of the target compound 2-6 with a yield of 13.3%, 0.184 g of the target compound 3-6 with a yield of 13.5%, and 0.113 g of the target compound 4-6 with a yield of 8.57%.

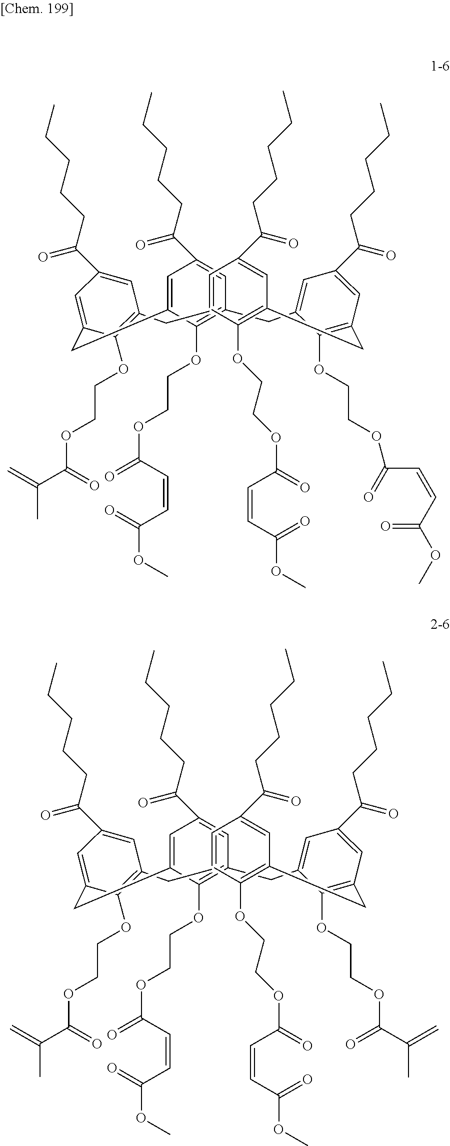

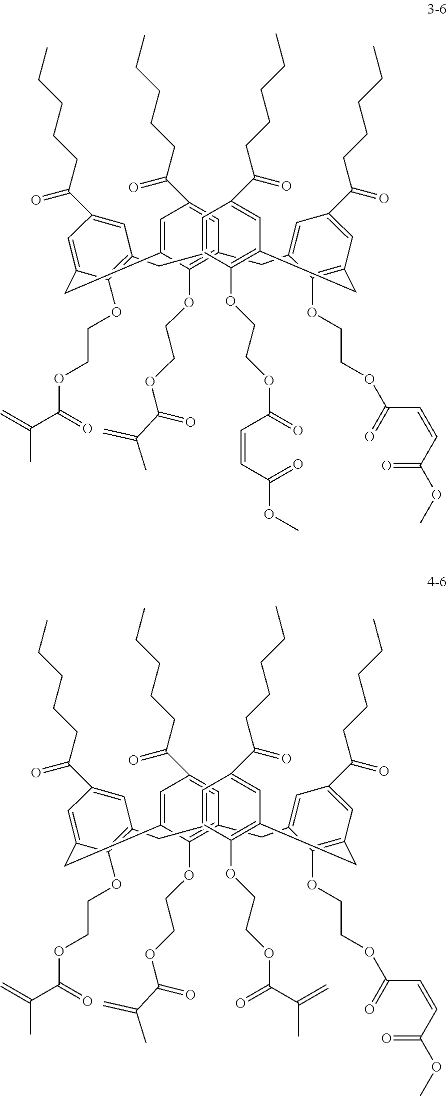

### Example 2

The same procedure as in Example 1 was repeated except that D-4 was used instead of D-6, to thereby obtain 0.392 g of the target compound 1-4 with a yield of 26.3%, 0.180 g of the target compound 2-4 with a yield of 12.5%, 0.176 g of the target compound 3-4 with a yield of 12.2%, and 0.111 g of the target compound 4-4 with a yield of 7.98%.

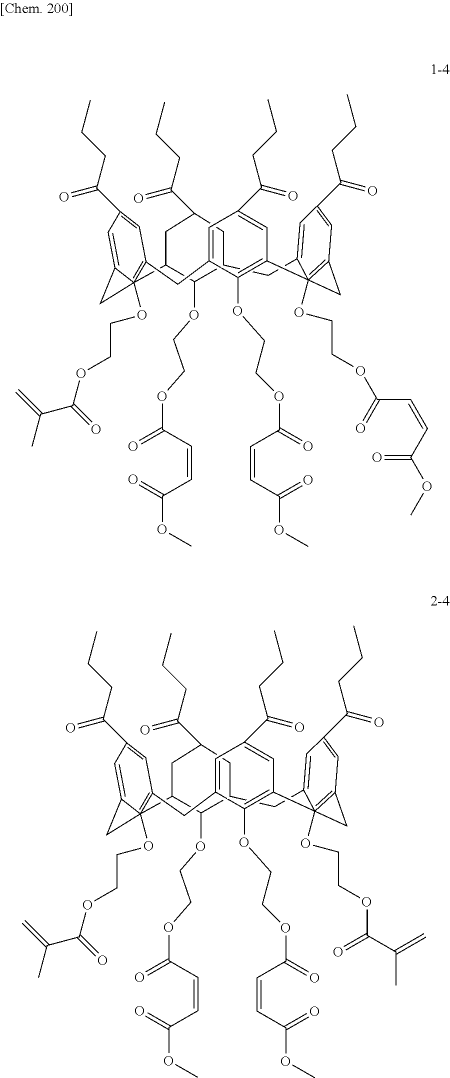

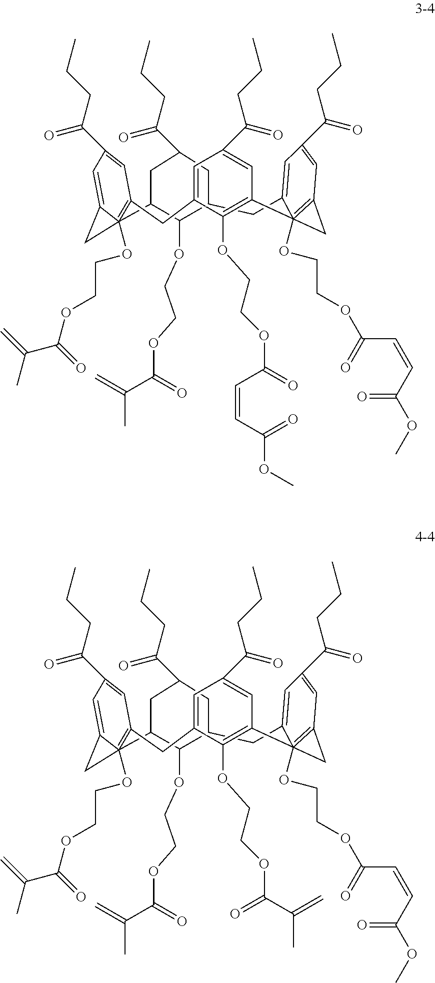

### Example 3

The same procedure as in Example 1 was repeated except that D-7 was used instead of D-6, to thereby obtain 0.410 g of the target compound 1-7 with a yield of 29.6%, 0.201 g of the target compound 2-7 with a yield of 15.0%, 0.196 g of the target compound 3-7 with a yield of 14.6%, and 0.131 g of the target compound 4-7 with a yield of 10.1%.

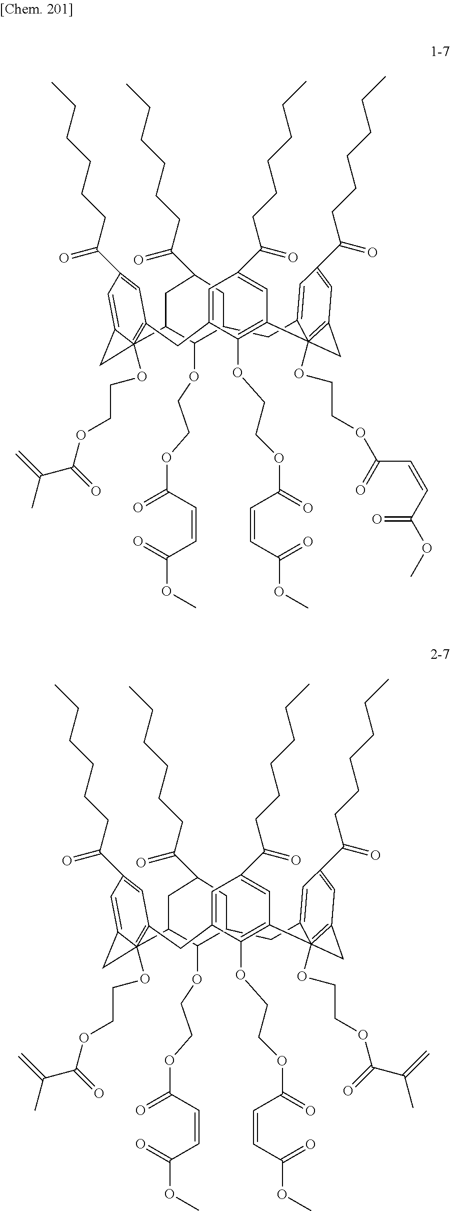

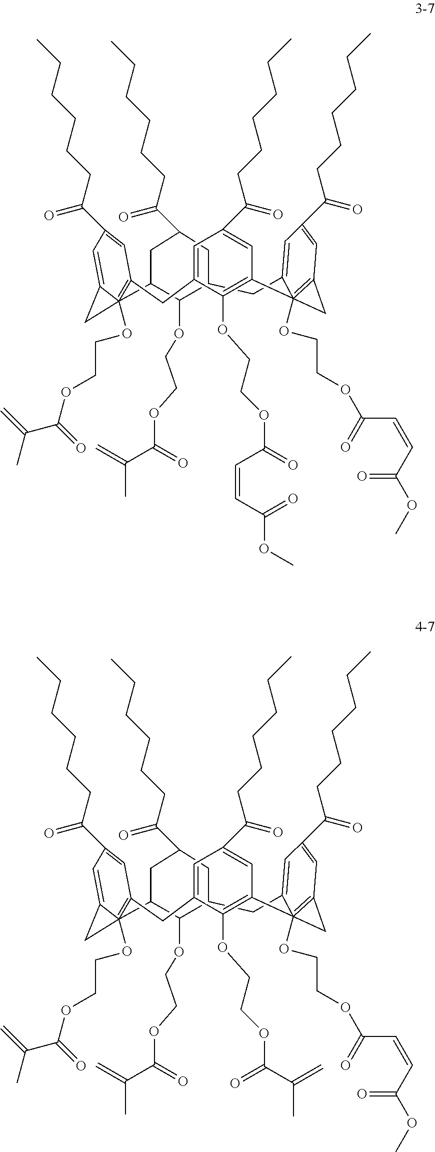

### Example 4

The same procedure as in Example 1 was repeated except that acrylic acid was used instead of methacrylic acid, to thereby obtain 0.401 g of the target compound 5-6 with a yield of 28.8%, 0.195 g of the target compound 6-6 with a yield of 14.6%, 0.189 g of the target compound 7-6 with a yield of 14.1%, and 0.118 g of the target compound 8-6 with a yield of 9.25%.

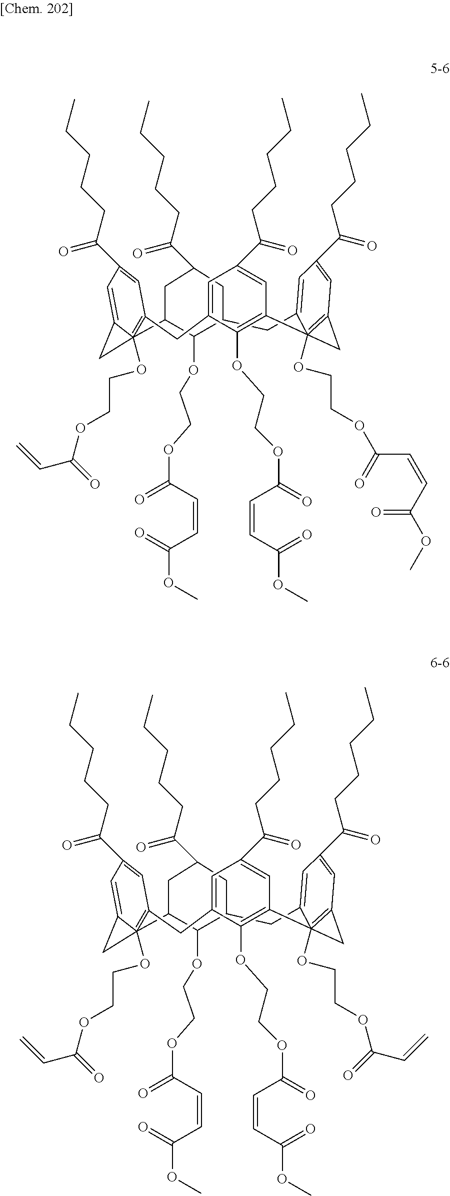

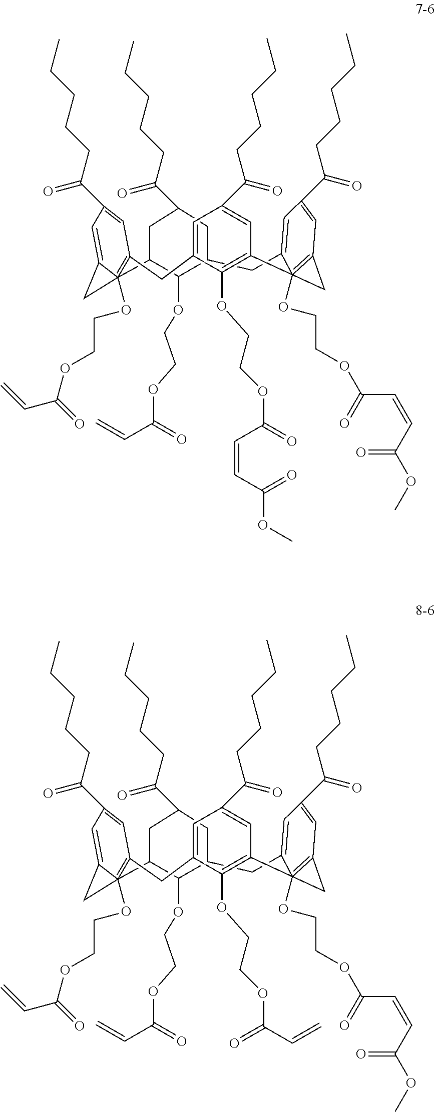

### Example 5

The same procedure as in Example 1 was repeated except that monoethyl maleate was used instead of monomethyl maleate, to thereby obtain 0.389 g of the target compound 9-6 with a yield of 26.8%, 0.181 g of the target compound 10-6 with a yield of 13.1%, 0.179 g of the target compound 11-6 with a yield of 12.9%, and 0.115 g of the target compound 12-6 with a yield of 8.63%.

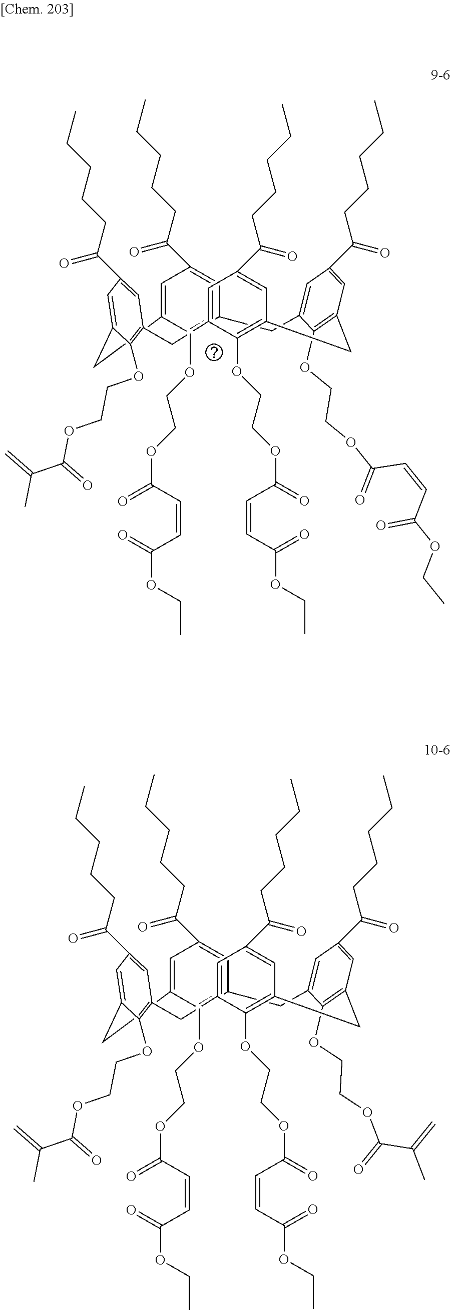

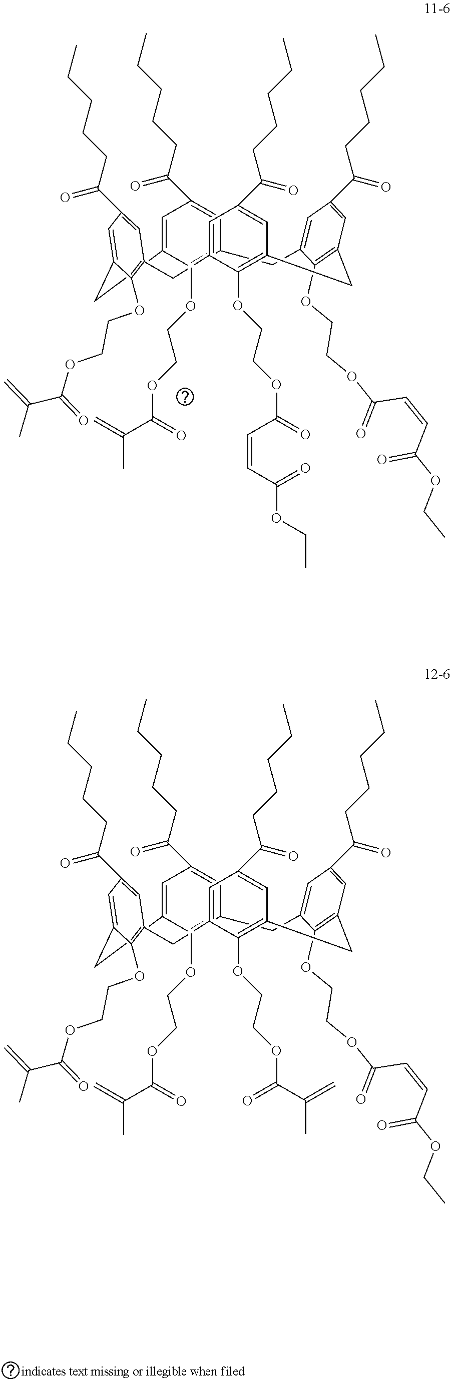

### Example 6

The same procedure as in Example 5 was repeated except that acrylic acid was used instead of methacrylic acid, to thereby obtain 0.389 g of the target compound 9-6 with a yield of 27.1%, 0.178 g of the target compound 10-6 with a yield of 13.1%, 0.176 g of the target compound 11-6 with a yield of 12.9%, and 0.104 g of the target compound 12-6 with a yield of 8.06%.

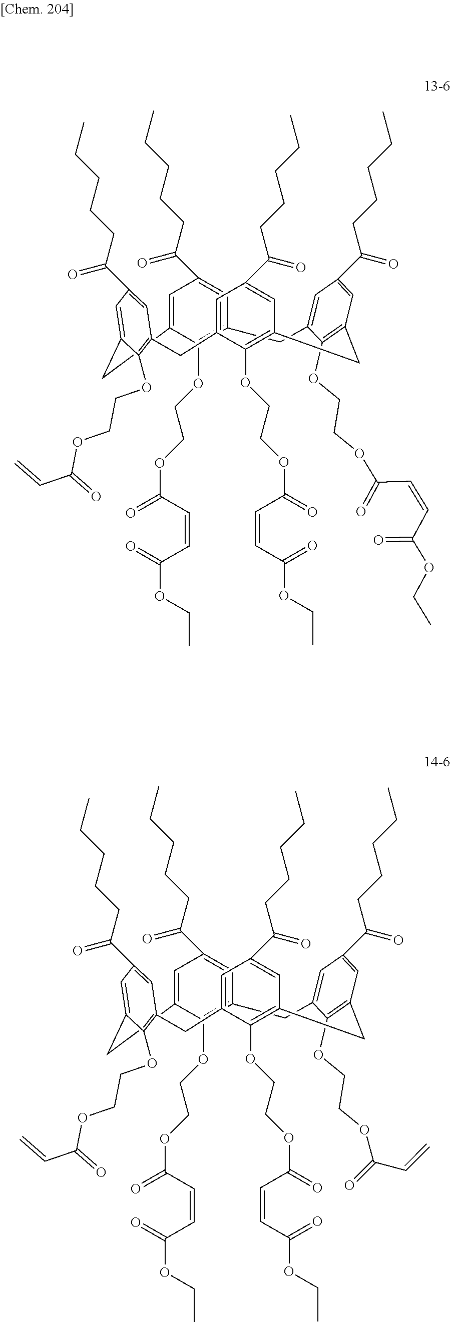

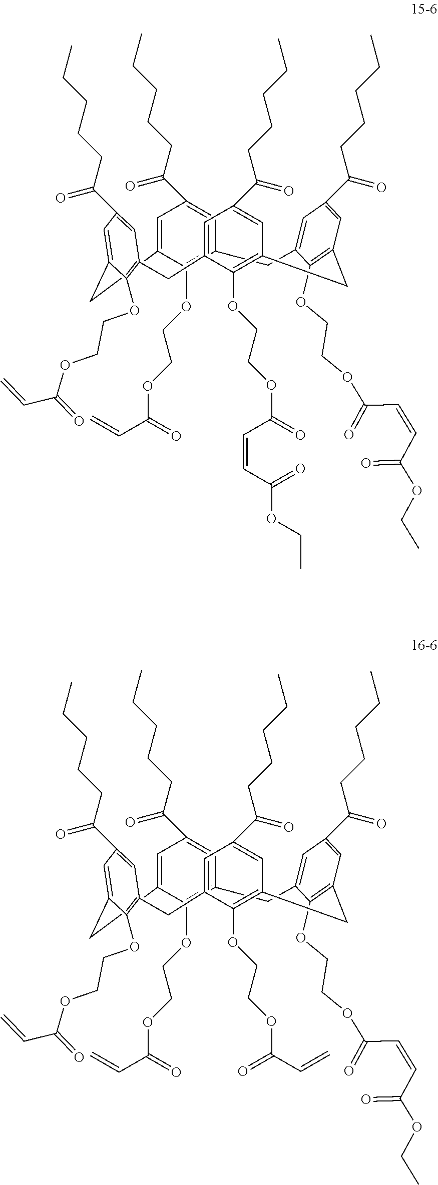

### Synthesis Example 13

The same procedure as in Synthesis Example 6 was repeated except that methyl bromopyopionate was used instead of methyl bromoacetate, to thereby obtain 4.307 g of compound E-6 represented by the following structural formula. The yield was 60.6%.

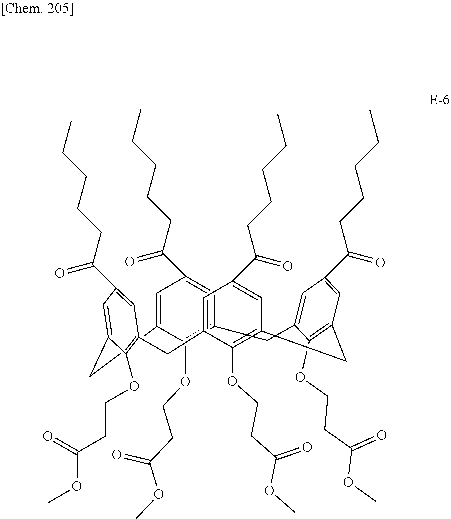 [SMILES: CCCCCC(=O)c1cc2c(OCCC(=O)OC)c(c1)Cc1cc(C(=O)CCCCC)cc(c1OCCC(=O)OC)Cc1cc(C(=O)CCCCC)cc(c1OCCC(=O)OC)Cc1cc(C(=O)CCCCC)cc(c1OCCC(=O)OC)C2]

### Synthesis Example 14

The same procedure as in Synthesis Example 10 was repeated except that E-6 was used instead of C-6, to thereby obtain 2.989 g of compound F-6 represented by the following structural formula. The yield was 80.6%.

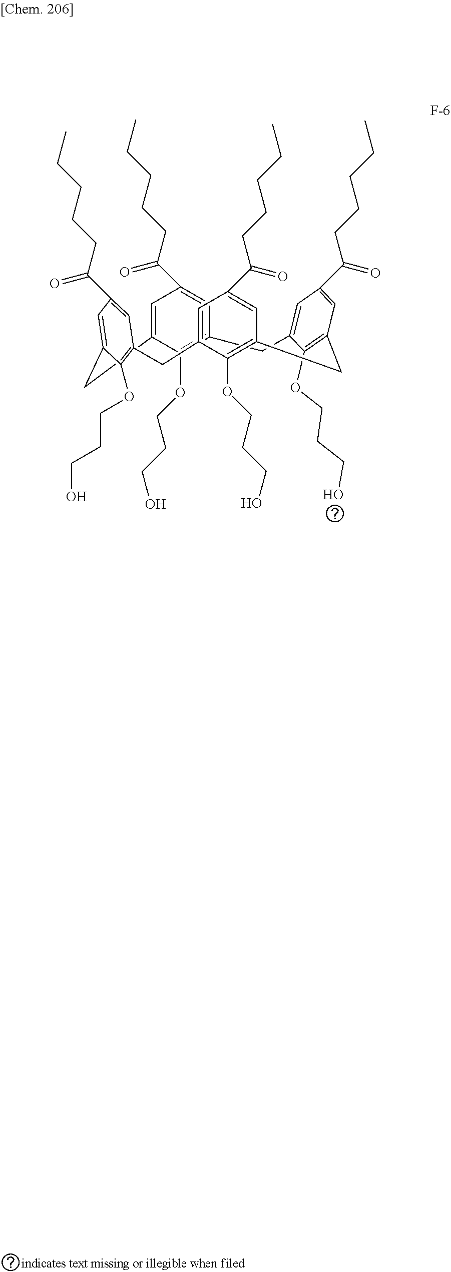 [SMILES: CCCCCC(=O)c1cc2c(OCCCO)c(c1)Cc1cc(C(=O)CCCCC)cc(c1OCCCO)Cc1cc(C(=O)CCCCC)cc(c1OCCCO)Cc1cc(C(=O)CCCCC)cc(c1OCCCO)C2]

### Example 7

The same procedure as in Example 1 was repeated except that F-6 was used instead of D-6, to thereby obtain 0.408 g of the target compound 17-6 with a yield of 29.4%, 0.201 g of the target compound 18-6 with a yield of 15.0%, 0.199 g of the target compound 19-6 with a yield of 14.8%, and 0.113 g of the target compound 20-6 with a yield of 8.68%.

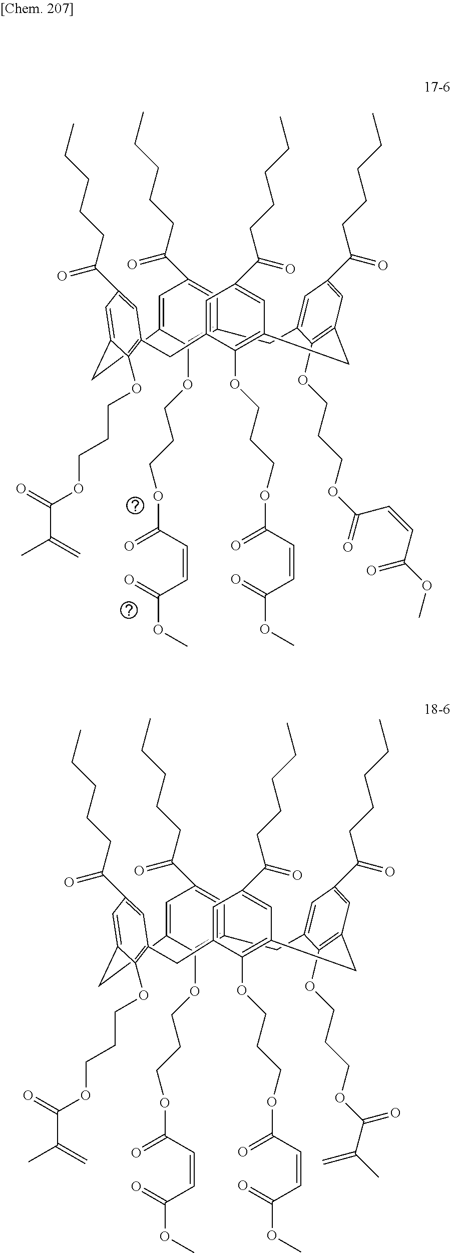 [SMILES: C=C(C)C(=O)OCCCOc1c2cc(C(=O)CCCCC)cc1Cc1cc(C(=O)CCCCC)cc(c1OCCCOC(=O)/C=C\C(=O)OC)Cc1cc(C(=O)CCCCC)cc(c1OCCCOC(=O)/C=C\C(=O)OC)Cc1cc(C(=O)CCCCC)cc(c1OCCCOC(=O)/C=C\C(=O)OC)C2 ; C=C(C)C(=O)OCCCOc1c2cc(C(=O)CCCCC)cc1Cc1cc(C(=O)CCCCC)cc(c1OCCCOC(=O)/C=C\C(=O)OC)Cc1cc(C(=O)CCCCC)cc(c1OCCCOC(=O)C(=C)C)Cc1cc(C(=O)CCCCC)cc(c1OCCCOC(=O)/C=C\C(=O)OC)C2]

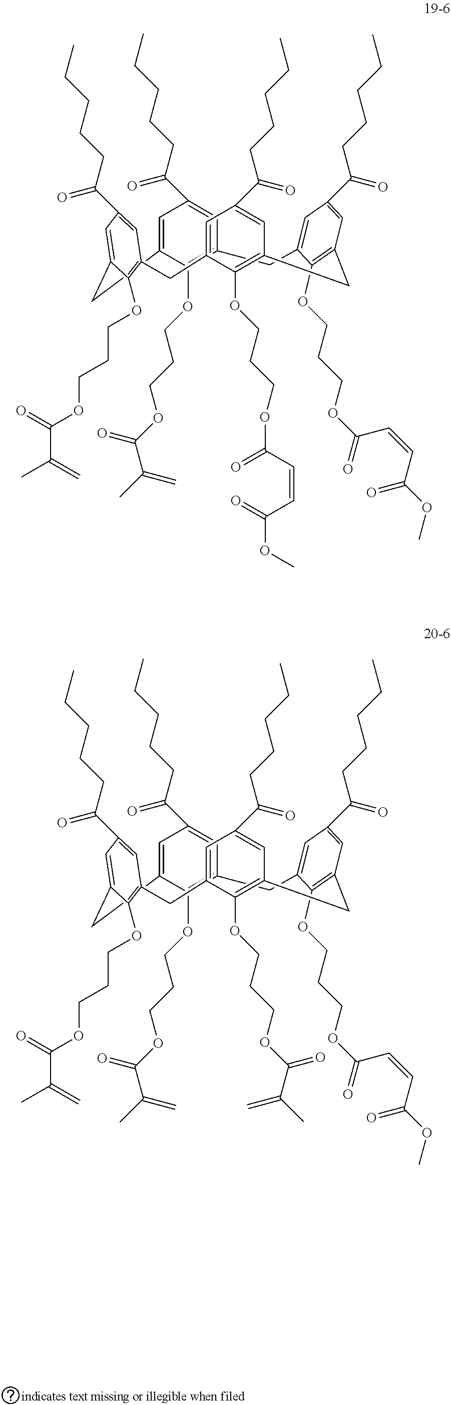 [SMILES: C=C(C)C(=O)OCCCOc1c2cc(C(=O)CCCCC)cc1Cc1cc(C(=O)CCCCC)cc(c1OCCCOC(=O)C(=C)C)Cc1cc(C(=O)CCCCC)cc(c1OCCCOC(=O)/C=C\C(=O)OC)Cc1cc(C(=O)CCCCC)cc(c1OCCCOC(=O)/C=C\C(=O)OC)C2 ; C=C(C)C(=O)OCCCOc1c2cc(C(=O)CCCCC)cc1Cc1cc(C(=O)CCCCC)cc(c1OCCCOC(=O)C(=C)C)Cc1cc(C(=O)CCCCC)cc(c1OCCCOC(=O)C(=C)C)Cc1cc(C(=O)CCCCC)cc(c1OCCCOC(=O)/C=C\C(=O)OC)C2]

### Example 8

The same procedure as in Example 7 was repeated except that acrylic acid was used instead of methacrylic acid, to thereby obtain 0.389 g of the target compound 21-6 with a yield of 28.4%, 0.178 g of the target compound 22-6 with a yield of 13.5%, 0.167 g of the target compound 23-6 with a yield of 12.7%, and 0.106 g of the target compound 24-6 with a yield of 8.40%.

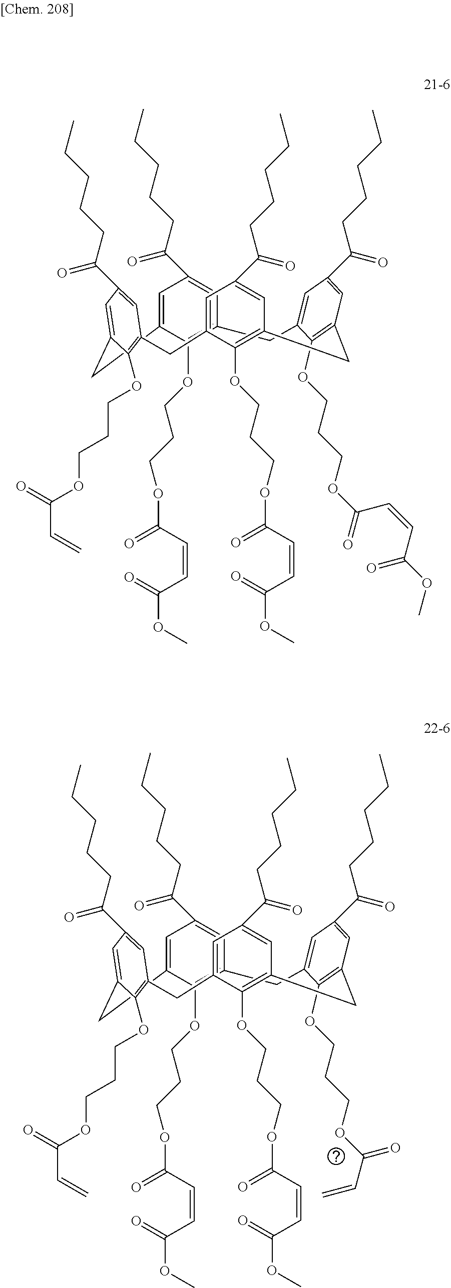 [SMILES: C=CC(=O)OCCCOc1c2cc(C(=O)CCCCC)cc1Cc1cc(C(=O)CCCCC)cc(c1OCCCOC(=O)/C=C\C(=O)OC)Cc1cc(C(=O)CCCCC)cc(c1OCCCOC(=O)/C=C\C(=O)OC)Cc1cc(C(=O)CCCCC)cc(c1OCCCOC(=O)/C=C\C(=O)OC)C2 ; C=CC(=O)OCCCOc1c2cc(C(=O)CCCCC)cc1Cc1cc(C(=O)CCCCC)cc(c1OCCCOC(=O)/C=C\C(=O)OC)Cc1cc(C(=O)CCCCC)cc(c1OCCCOC(=O)C=C)Cc1cc(C(=O)CCCCC)cc(c1OCCCOC(=O)/C=C\C(=O)OC)C2]

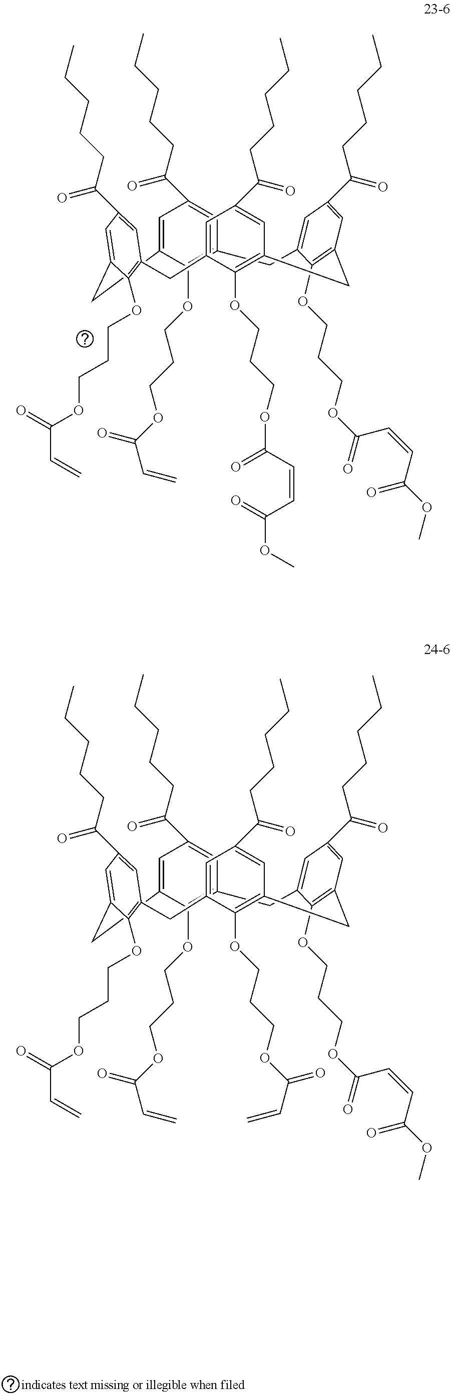 [SMILES: C=CC(=O)OCCCOc1c2cc(C(=O)CCCCC)cc1Cc1cc(C(=O)CCCCC)cc(c1OCCCOC(=O)/C=C\C(=O)OC)Cc1cc(C(=O)CCCCC)cc(c1OCCCOC(=O)/C=C\C(=O)OC)Cc1cc(C(=O)CCCCC)cc(c1OCCCOC(=O)C=C)C2 ; C=CC(=O)OCCCOc1c2cc(C(=O)CCCCC)cc1Cc1cc(C(=O)CCCCC)cc(c1OCCCOC(=O)C=C)Cc1cc(C(=O)CCCCC)cc(c1OCCCOC(=O)/C=C\C(=O)OC)Cc1cc(C(=O)CCCCC)cc(c1OCCCOC(=O)C=C)C2]

### Example 9

The same procedure as in Example 7 was repeated except that monoethyl maleate was used instead of monomethyl maleate, to thereby obtain 0.401 g of the target compound 25-6 with a yield of 28.4%, 0.201 g of the target compound 26-6 with a yield of 14.7%, 0.178 g of the target compound 27-6 with a yield of 13.0%, and 0.111 g of the target compound 28-6 with a yield of 8.44%.

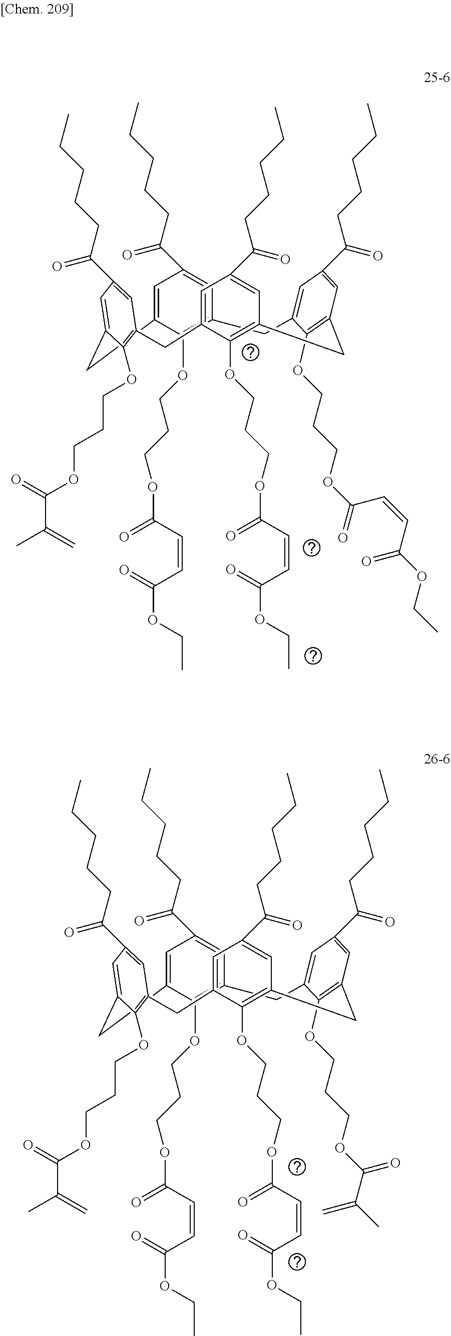 [SMILES: C=C(C)C(=O)OCCCOc1c2cc(C(=O)CCCCC)cc1Cc1cc(C(=O)CCCCC)cc(c1OCCCOC(=O)/C=C\C(=O)OCC)Cc1cc(C(=O)CCCCC)cc(c1OCCCOC(=O)/C=C\C(=O)OCC)Cc1cc(C(=O)CCCCC)cc(c1OCCCOC(=O)/C=C\C(=O)OCC)C2 ; C=C(C)C(=O)OCCCOc1c2cc(C(=O)CCCCC)cc1Cc1cc(C(=O)CCCCC)cc(c1OCCCOC(=O)/C=C\C(=O)OCC)Cc1cc(C(=O)CCCCC)cc(c1OCCCOC(=O)C(=C)C)Cc1cc(C(=O)CCCCC)cc(c1OCCCOC(=O)/C=C\C(=O)OCC)C2]

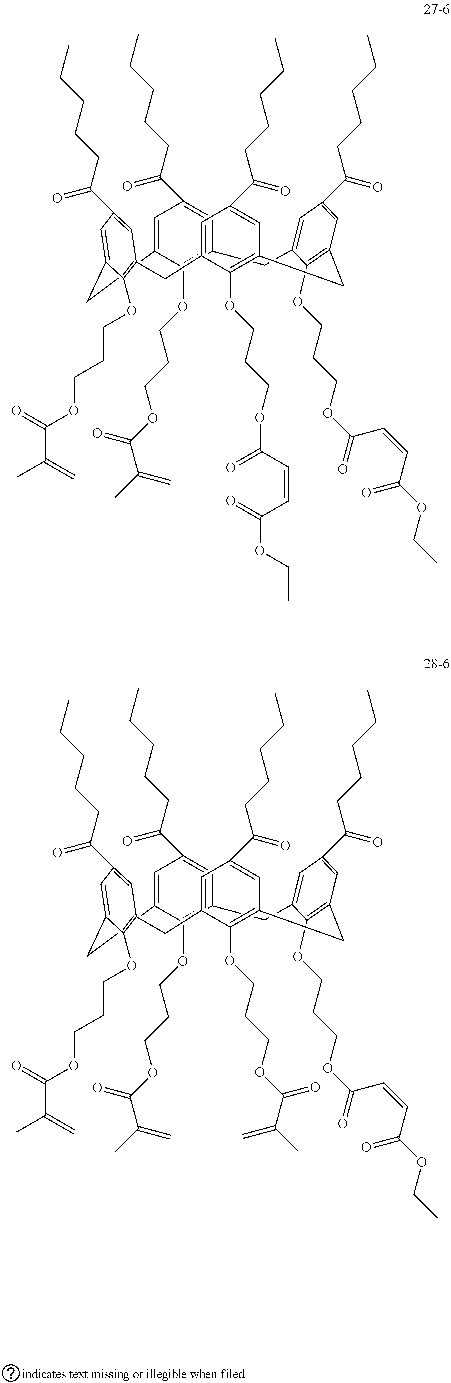 [SMILES: C=C(C)C(=O)OCCCOc1c2cc(C(=O)CCCCC)cc1Cc1cc(C(=O)CCCCC)cc(c1OCCCOC(=O)C(=C)C)Cc1cc(C(=O)CCCCC)cc(c1OCCCOC(=O)/C=C\C(=O)OCC)Cc1cc(C(=O)CCCCC)cc(c1OCCCOC(=O)/C=C\C(=O)OCC)C2 ; C=C(C)C(=O)OCCCOc1c2cc(C(=O)CCCCC)cc1Cc1cc(C(=O)CCCCC)cc(c1OCCCOC(=O)C(=C)C)Cc1cc(C(=O)CCCCC)cc(c1OCCCOC(=O)C(=C)C)Cc1cc(C(=O)CCCCC)cc(c1OCCCOC(=O)/C=C\C(=O)OCC)C2]

### Example 10

The same procedure as in Example 9 was repeated except that acrylic acid was used instead of methacrylic acid, to thereby obtain 0.391 g of the target compound 29-6 with a yield of 28.0%, 0.188 g of the target compound 30-6 with a yield of 14.0%, 0.189 g of the target compound 31-6 with a yield of 14.1%, and 0.101 g of the target compound 32-6 with a yield of 7.92%.

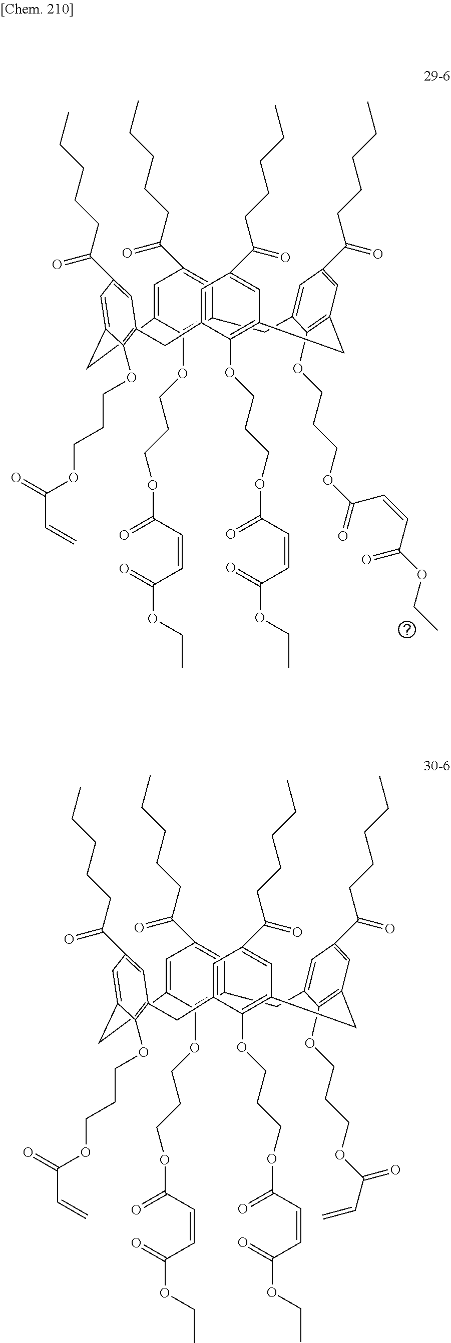 [SMILES: C=CC(=O)OCCCOc1c2cc(C(=O)CCCCC)cc1Cc1cc(C(=O)CCCCC)cc(c1OCCCOC(=O)/C=C\C(=O)OCC)Cc1cc(C(=O)CCCCC)cc(c1OCCCOC(=O)/C=C\C(=O)OCC)Cc1cc(C(=O)CCCCC)cc(c1OCCCOC(=O)/C=C\C(=O)OCC)C2 ; C=CC(=O)OCCCOc1c2cc(C(=O)CCCCC)cc1Cc1cc(C(=O)CCCCC)cc(c1OCCCOC(=O)/C=C\C(=O)OCC)Cc1cc(C(=O)CCCCC)cc(c1OCCCOC(=O)C=C)Cc1cc(C(=O)CCCCC)cc(c1OCCCOC(=O)/C=C\C(=O)OCC)C2]

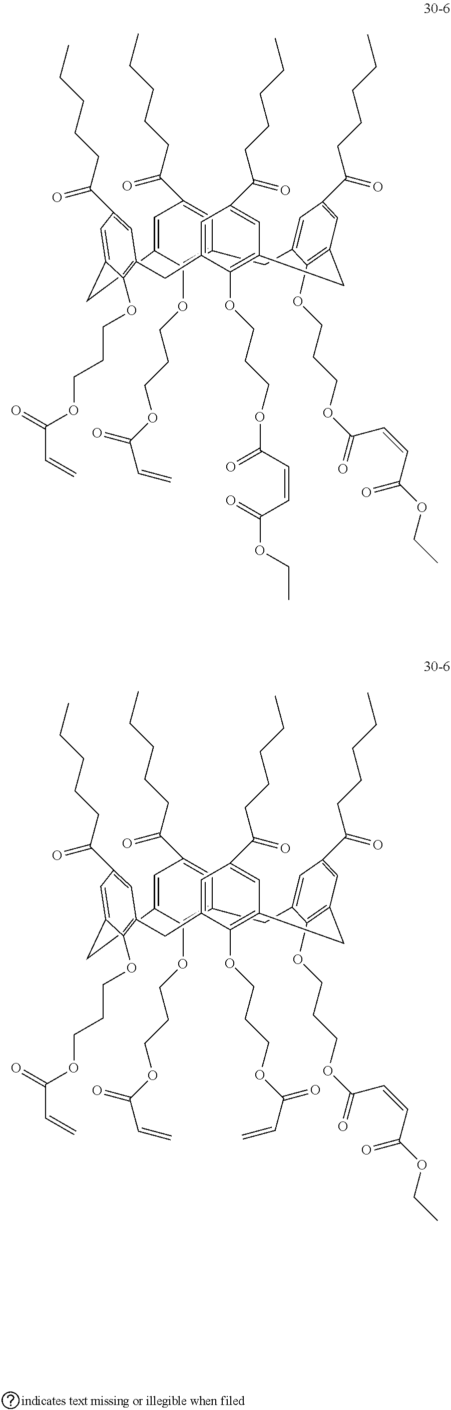 [SMILES: C=CC(=O)OCCCOc1c2cc(C(=O)CCCCC)cc1Cc1cc(C(=O)CCCCC)cc(c1OCCCOC(=O)/C=C\C(=O)OCC)Cc1cc(C(=O)CCCCC)cc(c1OCCCOC(=O)/C=C\C(=O)OCC)Cc1cc(C(=O)CCCCC)cc(c1OCCCOC(=O)C=C)C2 ; C=CC(=O)OCCCOc1c2cc(C(=O)CCCCC)cc1Cc1cc(C(=O)CCCCC)cc(c1OCCCOC(=O)C=C)Cc1cc(C(=O)CCCCC)cc(c1OCCCOC(=O)/C=C\C(=O)OCC)Cc1cc(C(=O)CCCCC)cc(c1OCCCOC(=O)C=C)C2]

### Synthesis Example 15

A 500 mL four-necked flask equipped with a stirrer, a thermometer, and a reflux condenser tube was charged with 92.6 g (113.33 mmol) of B-6 and 944.52 g of diethylene glycol monomethyl ether, and the mixture was stirred. Then 46.4 mL (906.64 mmol) of hydrazine monohydrate and 50.9 g (906.64 mmol) of potassium hydroxide pellets were added. The mixture was stirred at 100° C. for 30 minutes and further heat-refluxed for 8 hours. After completion of the reaction, the mixture was cooled to 90° C. Then 92.6 mL of ion exchanged water was added, and the resulting mixture was cooled to room temperature. The solution mixture was transferred to a beaker. 6N hydrochloric acid was added until the pH was 1, and 300 g of chloroform was added. The reaction mixture was transferred to a separatory funnel, and the organic layer was separated. Next, the aqueous layer was extracted with 300 g of chloroform three times, and the extract was combined with the organic layer. The organic layer was pre-dried over anhydrous magnesium sulfate, and the solvent was removed by evaporation using an evaporator to thereby obtain an orange viscus liquid. Methanol was added to reprecipitate crystals, and the generated white crystals were filtered and vacuum dried (at 60° C. for 6 hours or longer) to thereby obtain 54.34 g of compound G-6 represented by the following structural formula. The yield was 63.0%.

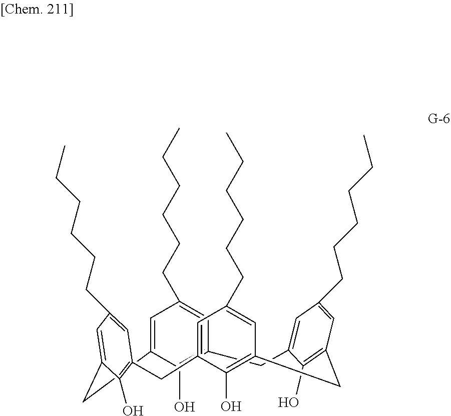 [SMILES: CCCCCCc1cc2c(O)c(c1)Cc1cc(CCCCCC)cc(c1O)Cc1cc(CCCCCC)cc(c1O)Cc1cc(CCCCCC)cc(c1O)C2]

### Synthesis Example 16

The same procedure as in Synthesis Example 15 was repeated except that B-4 was used instead of B-6, to thereby obtain 72.45 g of compound G-4 represented by the following structural formula. The yield was 83.1%.

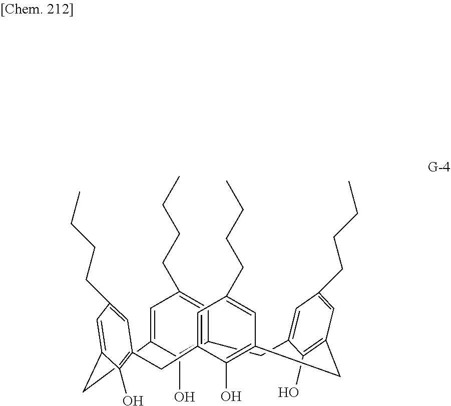 [SMILES: CCCCc1cc2c(O)c(c1)Cc1cc(CCCC)cc(c1O)Cc1cc(CCCC)cc(c1O)Cc1cc(CCCC)cc(c1O)C2]

### Synthesis Example 17

The same procedure as in Synthesis Example 15 was repeated except that B-7 was used instead of B-6, to thereby obtain 78.4 g of compound G-7 represented by the following structural formula. The yield was 82.7%.

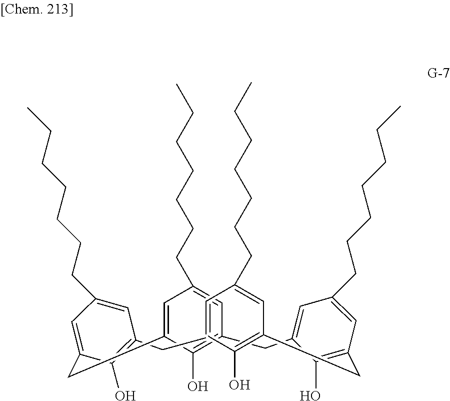

### Synthesis Example 18

The same procedure as in Synthesis Example 15 was repeated except that B-18 was used instead of B-6, to thereby obtain 37.9 g of compound G-18 represented by the following structural formula. The yield was 96.0%.

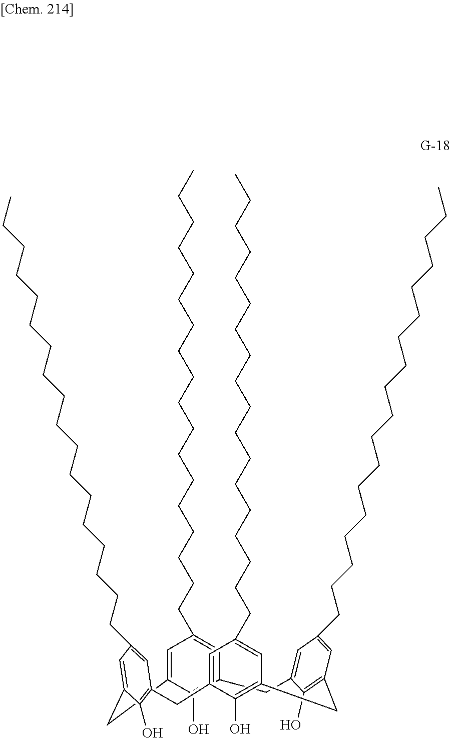 [SMILES: CCCCCCCCCCCCCCCCCCc1cc2c(O)c(c1)Cc1cc(CCCCCCCCCCCCCCCCCC)cc(c1O)Cc1cc(CCCCCCCCCCCCCCCCCC)cc(c1O)Cc1cc(CCCCCCCCCCCCCCCCCC)cc(c1O)C2]

### Synthesis Example 19

Referring to known documents (Tetrahedron Letters, 43(43), 7691-7693; 2002 and Tetrahedron Letters, 48(5), 905-12; 1992), compound G-1 represented by the following structural formula was synthesized according to the following two-step scheme (yield amount 75 g, yield 66.6%).

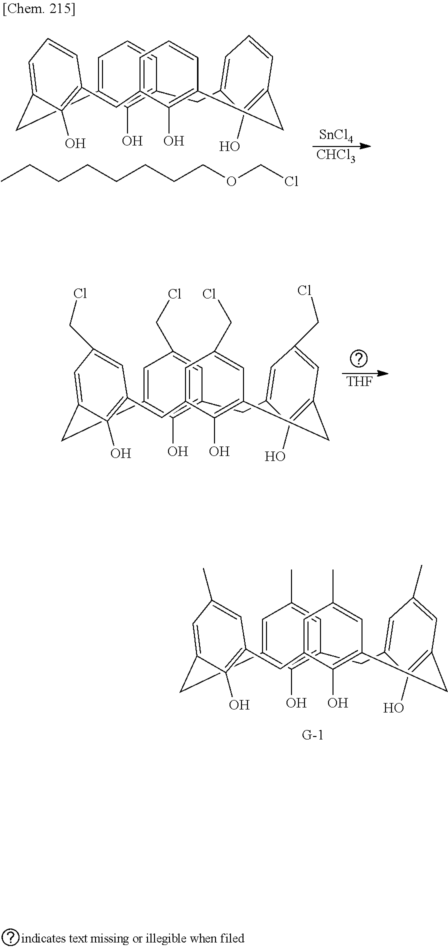

### Synthesis Example 20

A 1 L four-necked flask equipped with a stirrer, a thermometer, and a reflux condenser tube was charged with 20.00 g (26.276 mmol) of G-6, 400 g of acetonitrile, 15.29 g (105.11 mmol) of potassium carbonate, 10.511 g (10.511 mmol) of potassium iodide, and 32.158 g (210.21 mmol) of methyl 2-bromoacetate, and the mixture was allowed to react at 70° C. for 6 hours. The resulting mixture was cooled to room temperature, and ion exchanged water and 1N hydrochloric acid were added until the pH was 6. After 500 g of chloroform was added, the reaction mixture was transferred to a separatory funnel, and the organic layer was separated. The aqueous layer was extracted with 100 g of chloroform three times, and the extract was combined with the organic layer. The organic layer was pre-dried over anhydrous magnesium sulfate, and the solvent was removed by evaporation using an evaporator to thereby obtain a red waxy solid. The obtained red waxy solid was vacuum dried (at 60° C. for 6 hours or longer) to thereby obtain 21.67 g of compound H-6 represented by the following structural formula. The yield was 78.6%.

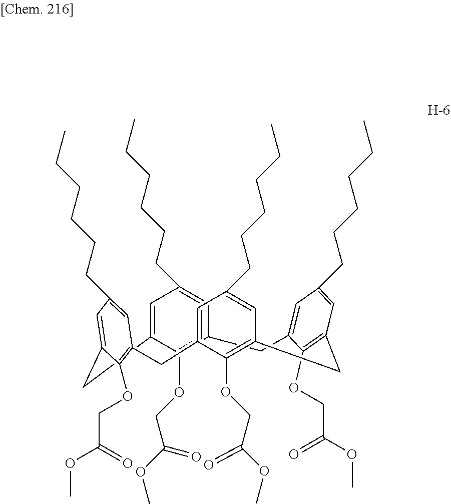 [SMILES: CCCCCCc1cc2c(OCC(=O)OC)c(c1)Cc1cc(CCCCCC)cc(c1OCC(=O)OC)Cc1cc(CCCCCC)cc(c1OCC(=O)OC)Cc1cc(CCCCCC)cc(c1OCC(=O)OC)C2]

### Synthesis Example 21

The same procedure as in Synthesis Example 20 was repeated except that G-4 was used instead of G-6, to thereby obtain 21.81 g of compound H-4 represented by the following structural formula. The yield was 75.5%.

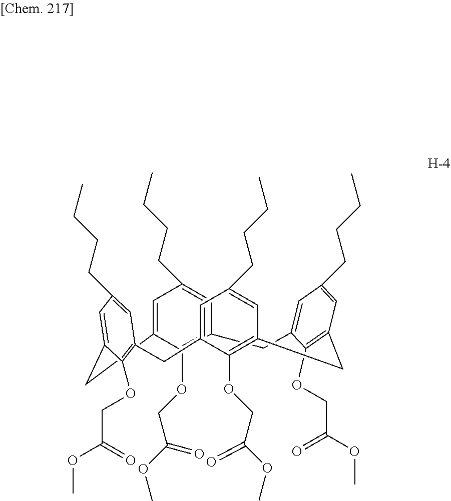 [SMILES: CCCCc1cc2c(OCC(=O)OC)c(c1)Cc1cc(CCCC)cc(c1OCC(=O)OC)Cc1cc(CCCC)cc(c1OCC(=O)OC)Cc1cc(CCCC)cc(c1OCC(=O)OC)C2]

### Synthesis Example 22

The same procedure as in Synthesis Example 20 was repeated except that G-7 was used instead of G-6, to thereby obtain 20.98 g of compound H-7 represented by the following structural formula. The yield was 77.5%.

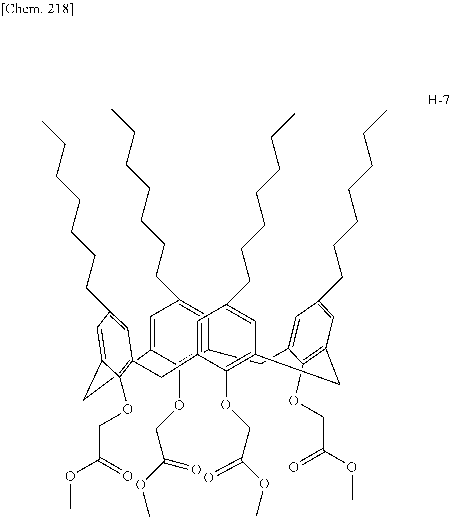 [SMILES: CCCCCCCc1cc2c(OCC(=O)OC)c(c1)Cc1cc(CCCCCCC)cc(c1OCC(=O)OC)Cc1cc(CCCCCCC)cc(c1OCC(=O)OC)Cc1cc(CCCCCCC)cc(c1OCC(=O)OC)C2]

### Synthesis Example 23

The same procedure as in Synthesis Example 20 was repeated except that G-18 was used instead of G-6, to thereby obtain 19.32 g of compound H-18 represented by the following structural formula. The yield was 80.4%.

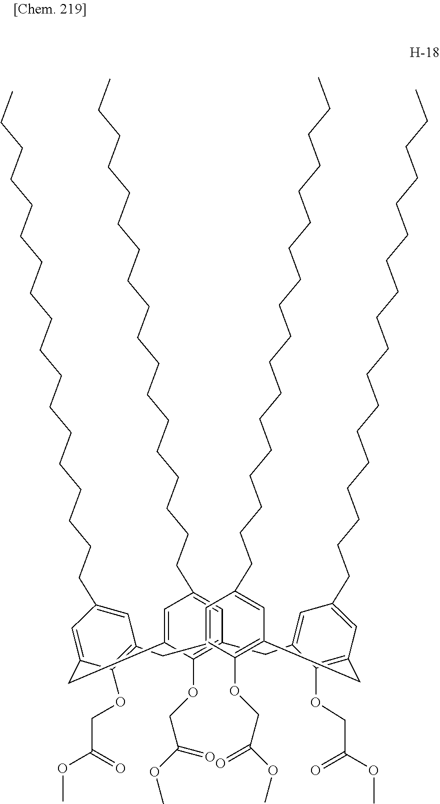

### Synthesis Example 24

The same procedure as in Synthesis Example 20 was repeated except that G-1 was used instead of G-6, to thereby obtain 18.32 g of compound H-1 represented by the following structural formula. The yield was 57.3%.

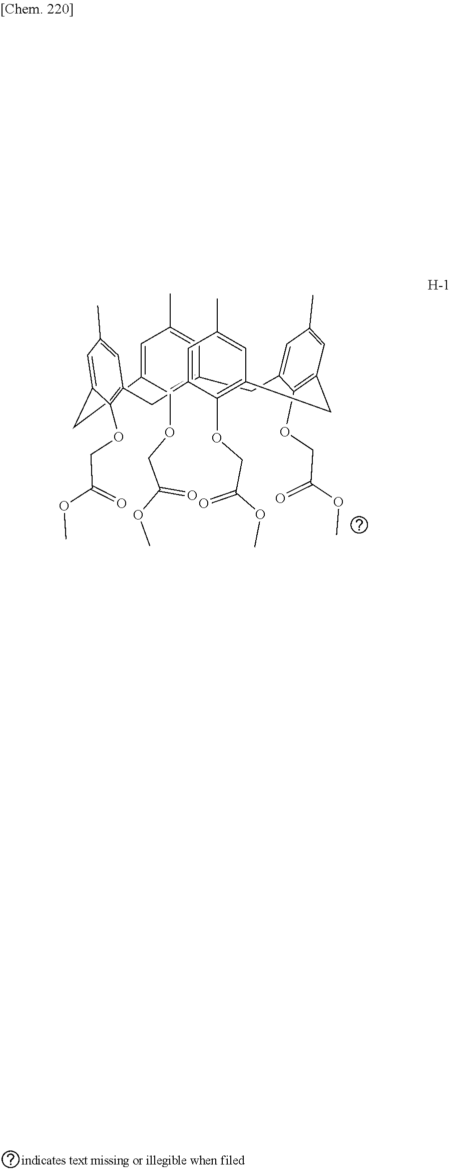 [SMILES: COC(=O)COc1c2cc(C)cc1Cc1cc(C)cc(c1OCC(=O)OC)Cc1cc(C)cc(c1OCC(=O)OC)Cc1cc(C)cc(c1OCC(=O)OC)C2]

### Synthesis Example 25

The same procedure as in Synthesis Example 10 was repeated except that H-6 was used instead of C-6, to thereby obtain 6.12 g of compound 1-6 represented by the following structural formula. The yield was 68.5%.

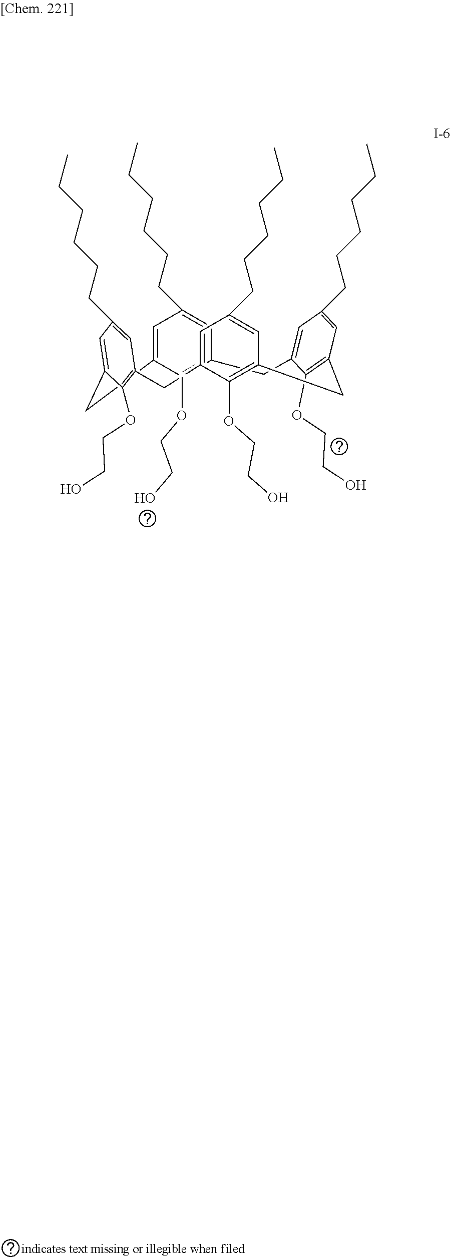 [SMILES: CCCCCCc1cc2c(OCCO)c(c1)Cc1cc(CCCCCC)cc(c1OCCO)Cc1cc(CCCCCC)cc(c1OCCO)Cc1cc(CCCCCC)cc(c1OCCO)C2]

### Synthesis Example 26

The same procedure as in Synthesis Example 25 was repeated except that H-4 was used instead of H-6, to thereby obtain 4.21 g of compound I-4 represented by the following structural formula. The yield was 81.4%.

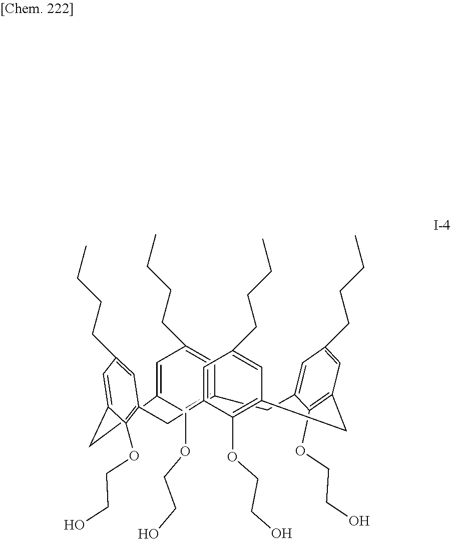 [SMILES: CCCCc1cc2c(OCCO)c(c1)Cc1cc(CCCC)cc(c1OCCO)Cc1cc(CCCC)cc(c1OCCO)Cc1cc(CCCC)cc(c1OCCO)C2]

### Synthesis Example 27

The same procedure as in Synthesis Example 25 was repeated except that H-7 was used instead of H-6, to thereby obtain 3.89 g of compound I-7 represented by the following structural formula. The yield was 84.5%.

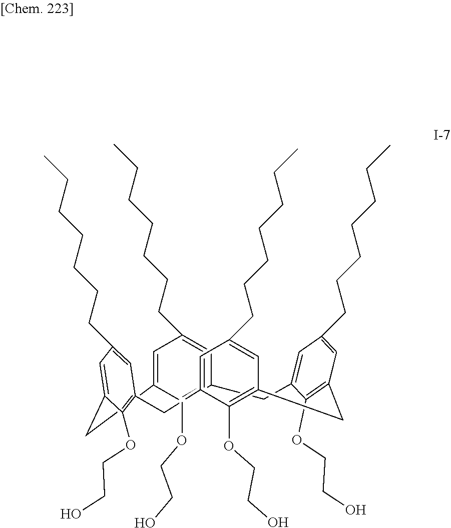 [SMILES: CCCCCCCc1cc2c(OCCO)c(c1)Cc1cc(CCCCCCC)cc(c1OCCO)Cc1cc(CCCCCCC)cc(c1OCCO)Cc1cc(CCCCCCC)cc(c1OCCO)C2]

### Synthesis Example 28

The same procedure as in Synthesis Example 25 was repeated except that H-18 was used instead of H-6, to thereby obtain 4.31 g of compound 1-18 represented by the following structural formula. The yield was 81.7%.

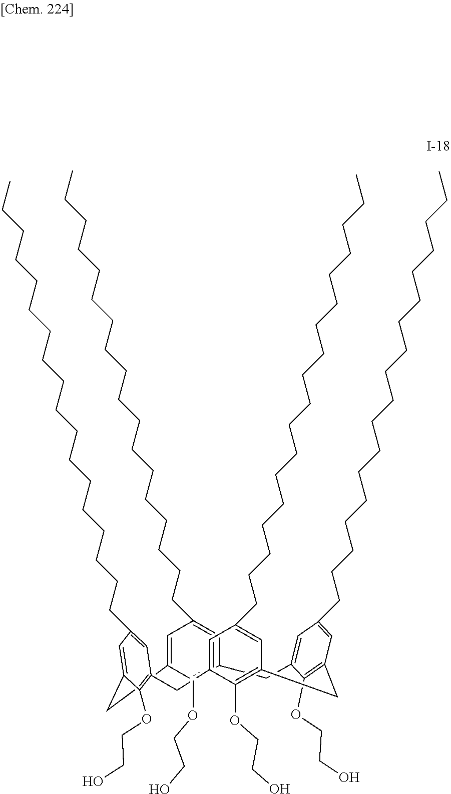 [SMILES: CCCCCCCCCCCCCCCCCCc1cc2c(OCCO)c(c1)Cc1cc(CCCCCCCCCCCCCCCCCC)cc(c1OCCO)Cc1cc(CCCCCCCCCCCCCCCCCC)cc(c1OCCO)Cc1cc(CCCCCCCCCCCCCCCCCC)cc(c1OCCO)C2]

### Synthesis Example 29

The same procedure as in Synthesis Example 25 was repeated except that H-1 was used instead of H-6, to thereby obtain 3.43 g of compound I-1 represented by the following structural formula. The yield was 85.1%.

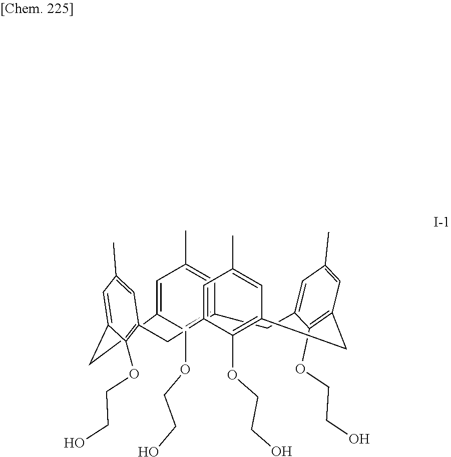 [SMILES: Cc1cc2c(OCCO)c(c1)Cc1cc(C)cc(c1OCCO)Cc1cc(C)cc(c1OCCO)Cc1cc(C)cc(c1OCCO)C2]

### Example 11

A 50 mL four-necked flask equipped with a stirrer, a thermometer, and a reflux condenser tube was charged with 1.00 g (1.067 mmol) of I-6, 3.077 g of tetrahydrofuran, 2.239 g (8.535 mmol) of triphenylphosphine, and 1.11 g (8.535 mmol) of monomethyl maleate, and the mixture was stirred. Next, 1.918 g (8.535 mmol) of diisopropyl azodicarboxylate diluted with 1.539 g of tetrahydrofuran was added dropwise to the mixture placed in an ice bath over 30 minutes. The resulting orange transparent reaction solution was stirred at room temperature for 10 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting red viscous liquid was subjected to column chromatography (eluent: n-hexane:ethyl acetate=85:15), and a light yellow transparent liquid was thereby obtained. The solvent was concentrated, and the concentrate was purified with methanol. The obtained viscous solid was vacuum dried (at 60° C. for 6 hours or longer) to thereby obtain 1.14 g of the target compound 33-6 with a yield of 77.1%.

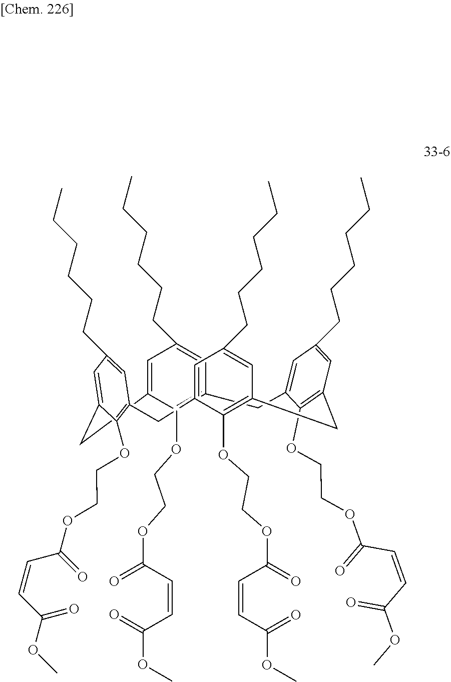 [SMILES: CCCCCCc1cc2c(OCCOC(=O)/C=C\C(=O)OC)c(c1)Cc1cc(CCCCCC)cc(c1OCCOC(=O)/C=C\C(=O)OC)Cc1cc(CCCCCC)cc(c1OCCOC(=O)/C=C\C(=O)OC)Cc1cc(CCCCCC)cc(c1OCCOC(=O)/C=C\C(=O)OC)C2]

### Example 12

The same procedure as in Example 11 was repeated except that I-4 was used instead of I-6, to thereby obtain 1.01 g of the target compound 33-7. The yield was 65.4%.

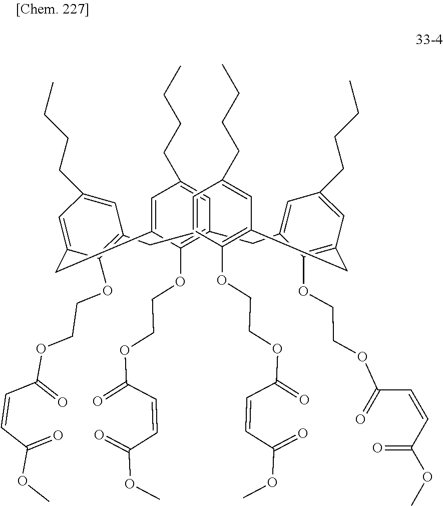

### Example 13

The same procedure as in Example 11 was repeated except that I-7 was used instead of I-6, to thereby obtain 1.14 g of the target compound 33-7. The yield was 78.6%.

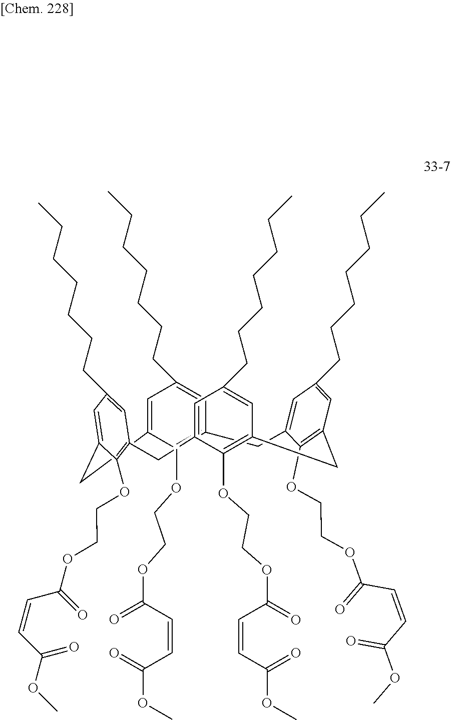 [SMILES: CCCCCCCc1cc2c(OCCOC(=O)/C=C\C(=O)OC)c(c1)Cc1cc(CCCCCCC)cc(c1OCCOC(=O)/C=C\C(=O)OC)Cc1cc(CCCCCCC)cc(c1OCCOC(=O)/C=C\C(=O)OC)Cc1cc(CCCCCCC)cc(c1OCCOC(=O)/C=C\C(=O)OC)C2]

### Example 14

The same procedure as in Example 11 was repeated except that I-18 was used instead of I-6, to thereby obtain 0.971 g of the target compound 33-18. The yield was 76.0%.

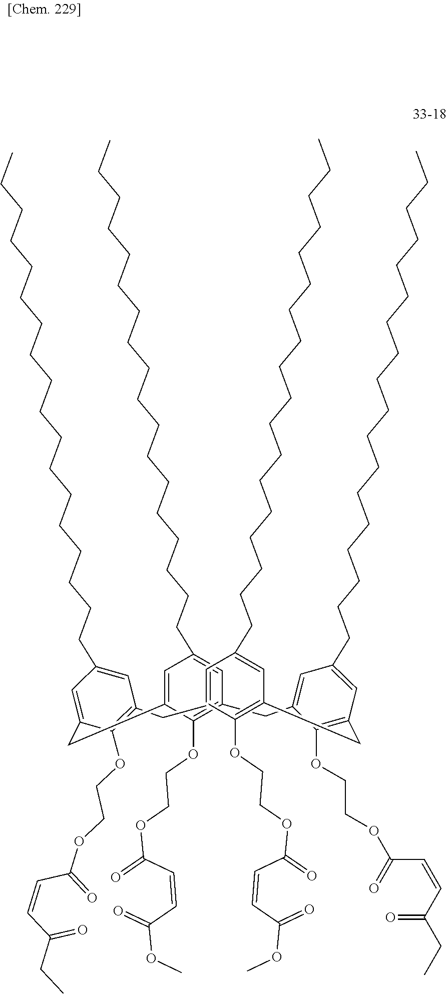

### Example 15

The same procedure as in Example 11 was repeated except that I-1 was used instead of I-6, to thereby obtain 0.871 g of the target compound 33-1. The yield was 51.8%.

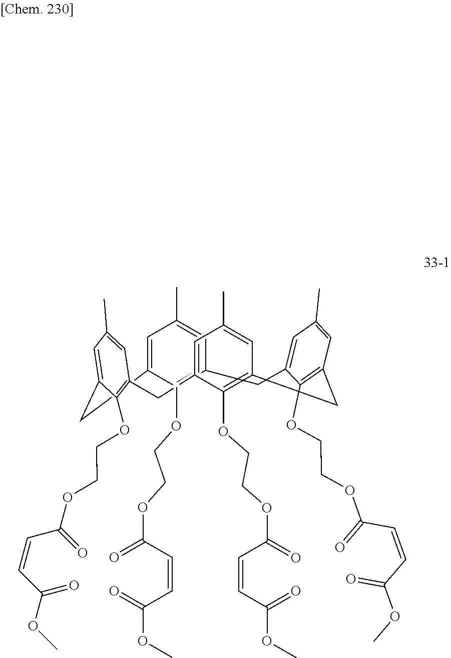 [SMILES: COC(=O)/C=C\C(=O)OCCOc1c2cc(C)cc1Cc1cc(C)cc(c1OCCOC(=O)/C=C\C(=O)OC)Cc1cc(C)cc(c1OCCOC(=O)/C=C\C(=O)OC)Cc1cc(C)cc(c1OCCOC(=O)/C=C\C(=O)OC)C2]

### Example 16

The same procedure as in Example 1 was repeated except that I-6 was used instead of D-6, to thereby obtain 0.433 g of the target compound 34-6 with a yield of 30.3%, 0.221 g of the target compound 35-6 with a yield of 16.0%, 0.218 g of the target compound 36-6 with a yield of 15.7%, and 0.151 g of the target compound 37-6 with a yield of 73.3%.

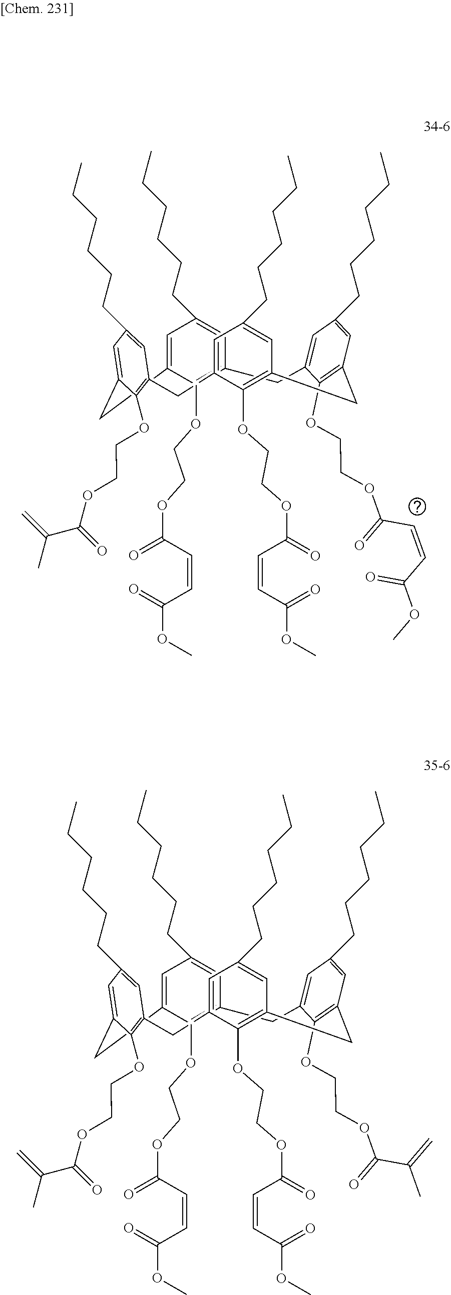 [SMILES: C=C(C)C(=O)OCCOc1c2cc(CCCCCC)cc1Cc1cc(CCCCCC)cc(c1OCCOC(=O)/C=C\C(=O)OC)Cc1cc(CCCCCC)cc(c1OCCOC(=O)/C=C\C(=O)OC)Cc1cc(CCCCCC)cc(c1OCCOC(=O)/C=C\C(=O)OC)C2 ; C=C(C)C(=O)OCCOc1c2cc(CCCCCC)cc1Cc1cc(CCCCCC)cc(c1OCCOC(=O)/C=C\C(=O)OC)Cc1cc(CCCCCC)cc(c1OCCOC(=O)C(=C)C)Cc1cc(CCCCCC)cc(c1OCCOC(=O)/C=C\C(=O)OC)C2]

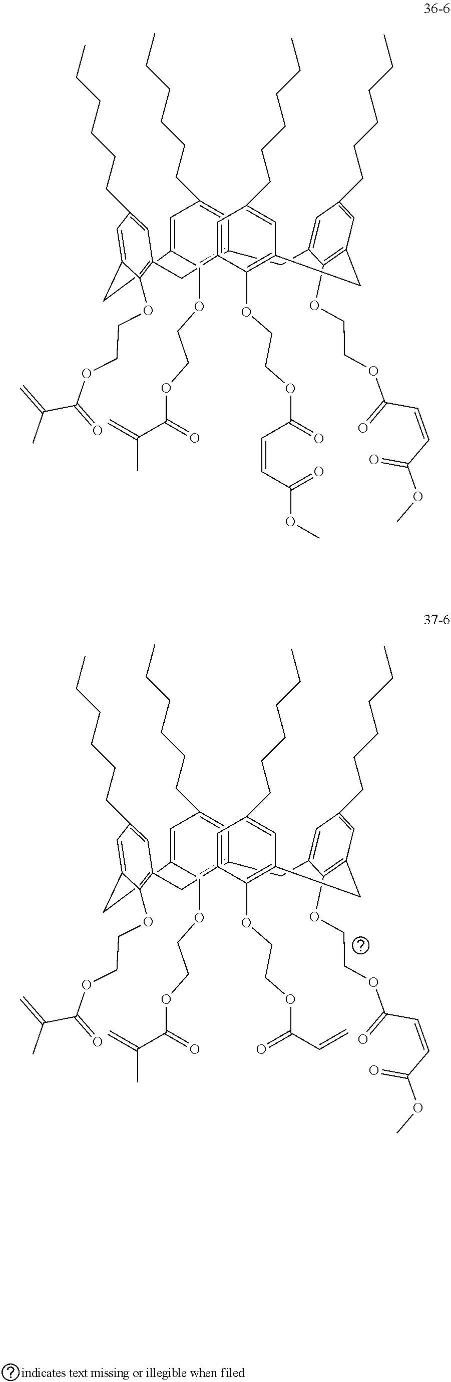 [SMILES: C=C(C)C(=O)OCCOc1c2cc(CCCCCC)cc1Cc1cc(CCCCCC)cc(c1OCCOC(=O)C(=C)C)Cc1cc(CCCCCC)cc(c1OCCOC(=O)/C=C\C(=O)OC)Cc1cc(CCCCCC)cc(c1OCCOC(=O)/C=C\C(=O)OC)C2 ; C=CC(=O)OCCOc1c2cc(CCCCCC)cc1Cc1cc(CCCCCC)cc(c1OCCOC(=O)C(=C)C)Cc1cc(CCCCCC)cc(c1OCCOC(=O)C(=C)C)Cc1cc(CCCCCC)cc(c1OCCOC(=O)/C=C\C(=O)OC)C2]

### Example 17

The same procedure as in Example 16 was repeated except that I-4 was used instead of I-6, to thereby obtain 0.425 g of the target compound 34-4 with a yield of 28.5%, 0.216 g of the target compound 35-4 with a yield of 15.0%, 0.221 g of the target compound 36-4 with a yield of 15.4%, and 0.123 g of the target compound 37-4 with a yield of 8.89%.

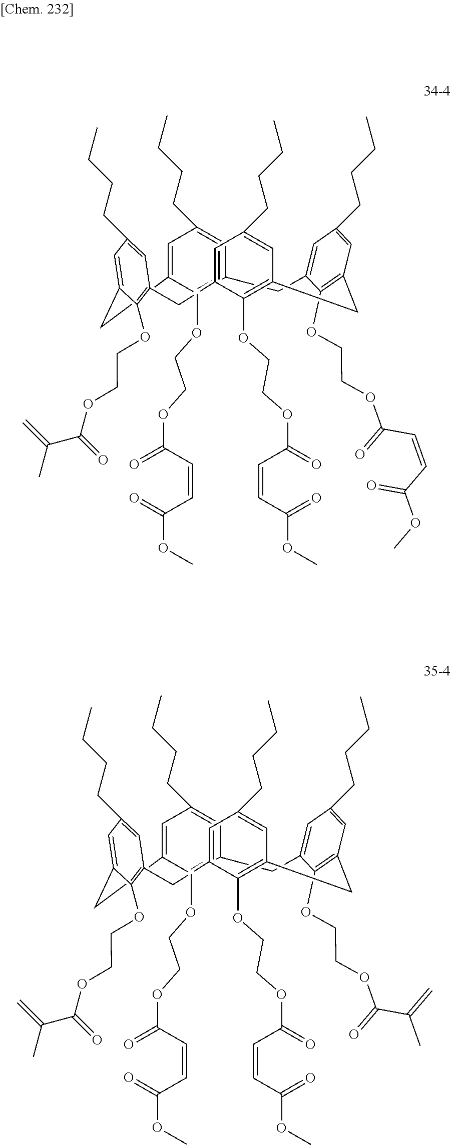 [SMILES: C=C(C)C(=O)OCCOc1c2cc(CCCC)cc1Cc1cc(CCCC)cc(c1OCCOC(=O)/C=C\C(=O)OC)Cc1cc(CCCC)cc(c1OCCOC(=O)/C=C\C(=O)OC)Cc1cc(CCCC)cc(c1OCCOC(=O)/C=C\C(=O)OC)C2 ; C=C(C)C(=O)OCCOc1c2cc(CCCC)cc1Cc1cc(CCCC)cc(c1OCCOC(=O)/C=C\C(=O)OC)Cc1cc(CCCC)cc(c1OCCOC(=O)C(=C)C)Cc1cc(CCCC)cc(c1OCCOC(=O)/C=C\C(=O)OC)C2]

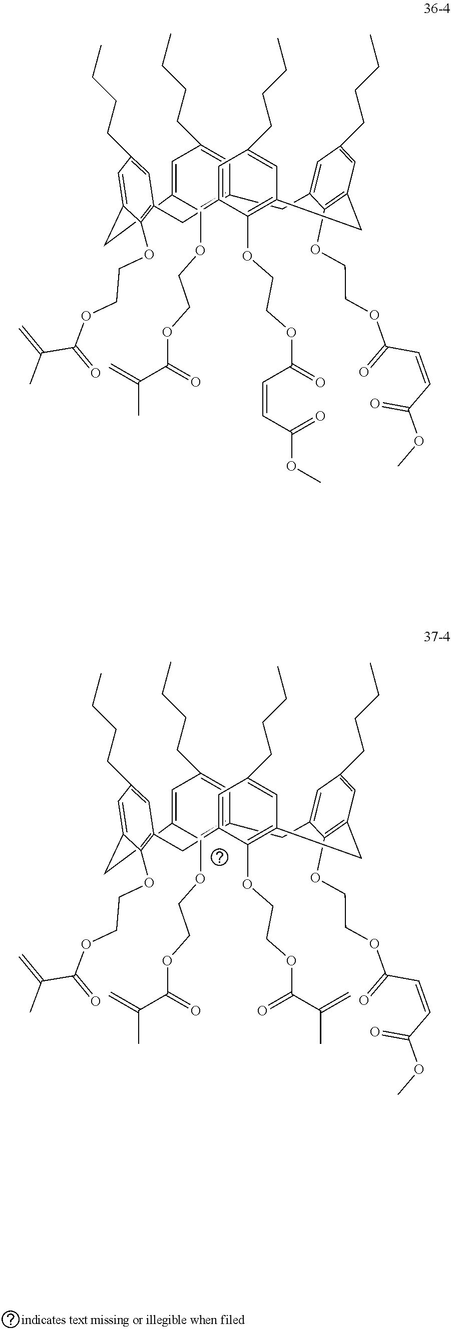 [SMILES: C=C(C)C(=O)OCCOc1c2cc(CCCC)cc1Cc1cc(CCCC)cc(c1OCCOC(=O)C(=C)C)Cc1cc(CCCC)cc(c1OCCOC(=O)/C=C\C(=O)OC)Cc1cc(CCCC)cc(c1OCCOC(=O)/C=C\C(=O)OC)C2 ; C=C(C)C(=O)OCCOc1c2cc(CCCC)cc1Cc1cc(CCCC)cc(c1OCCOC(=O)C(=C)C)Cc1cc(CCCC)cc(c1OCCOC(=O)C(=C)C)Cc1cc(CCCC)cc(c1OCCOC(=O)/C=C\C(=O)OC)C2]

### Example 18

The same procedure as in Example 16 was repeated except that I-7 was used instead of I-6, to thereby obtain 0.451 g of the target compound 34-7 with a yield of 32.1%, 0.228 g of the target compound 35-7 with a yield of 16.7%, 0.224 g of the target compound 36-7 with a yield of 16.4%, and 0.151 g of the target compound 37-7 with a yield of 11.5%.

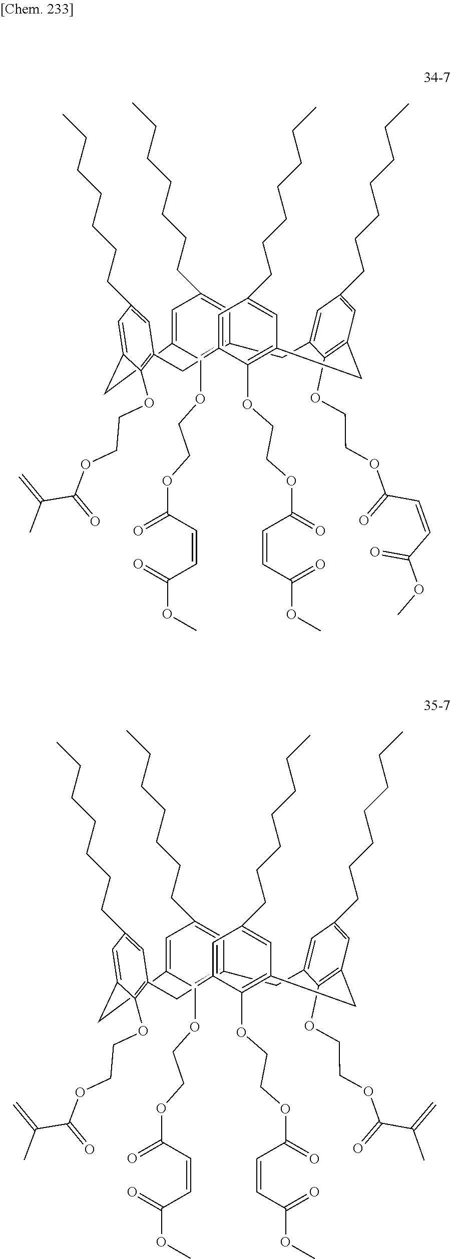 [SMILES: C=C(C)C(=O)OCCOc1c2cc(CCCCCCC)cc1Cc1cc(CCCCCCC)cc(c1OCCOC(=O)/C=C\C(=O)OC)Cc1cc(CCCCCCC)cc(c1OCCOC(=O)/C=C\C(=O)OC)Cc1cc(CCCCCCC)cc(c1OCCOC(=O)/C=C\C(=O)OC)C2 ; C=C(C)C(=O)OCCOc1c2cc(CCCCCCC)cc1Cc1cc(CCCCCCC)cc(c1OCCOC(=O)/C=C\C(=O)OC)Cc1cc(CCCCCCC)cc(c1OCCOC(=O)C(=C)C)Cc1cc(CCCCCCC)cc(c1OCCOC(=O)/C=C\C(=O)OC)C2]

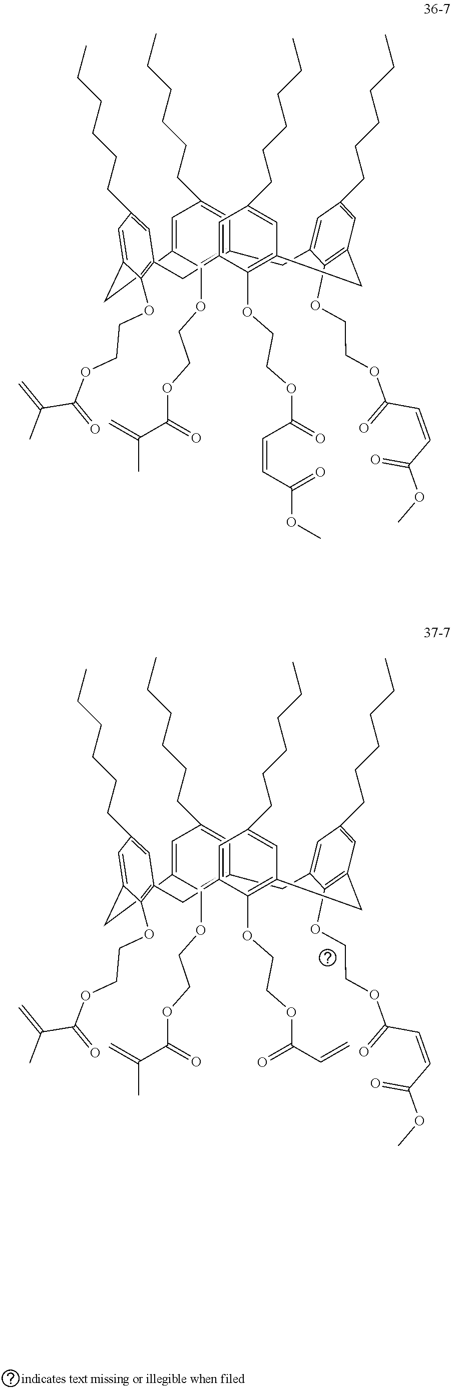 [SMILES: C=C(C)C(=O)OCCOc1c2cc(CCCCCC)cc1Cc1cc(CCCCCC)cc(c1OCCOC(=O)C(=C)C)Cc1cc(CCCCCC)cc(c1OCCOC(=O)/C=C\C(=O)OC)Cc1cc(CCCCCC)cc(c1OCCOC(=O)/C=C\C(=O)OC)C2 ; C=CC(=O)OCCOc1c2cc(CCCCCC)cc1Cc1cc(CCCCCC)cc(c1OCCOC(=O)C(=C)C)Cc1cc(CCCCCC)cc(c1OCCOC(=O)C(=C)C)Cc1cc(CCCCCC)cc(c1OCCOC(=O)/C=C\C(=O)OC)C2]

### Example 19

The same procedure as in Example 16 was repeated except that I-18 was used instead of I-6, to thereby obtain 0.421 g of the target compound 34-18 with a yield of 33.7%, 0.210 g of the target compound 35-18 with a yield of 17.1%, 0.195 g of the target compound 36-18 with a yield of 15.9%, and 0.124 g of the target compound 37-18 with a yield of 10.4%.

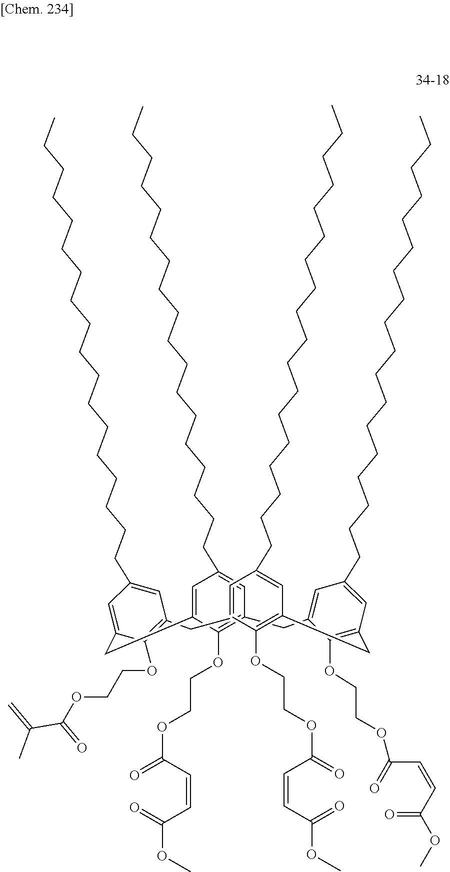

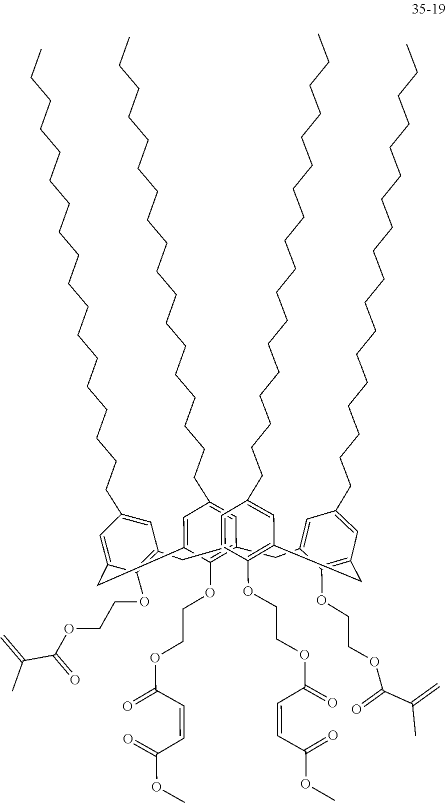

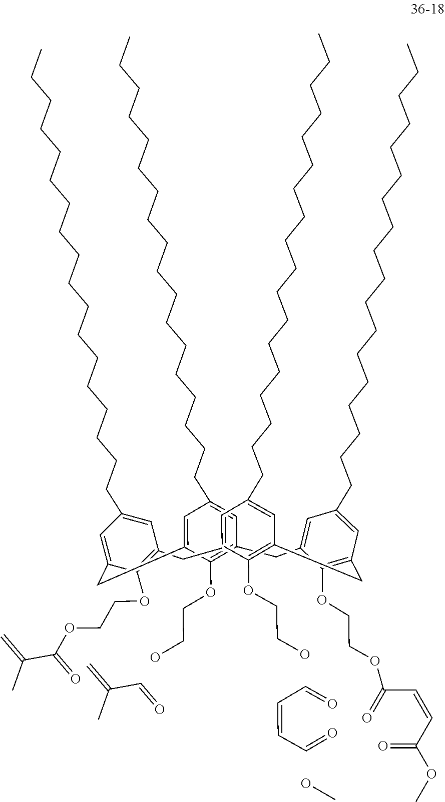

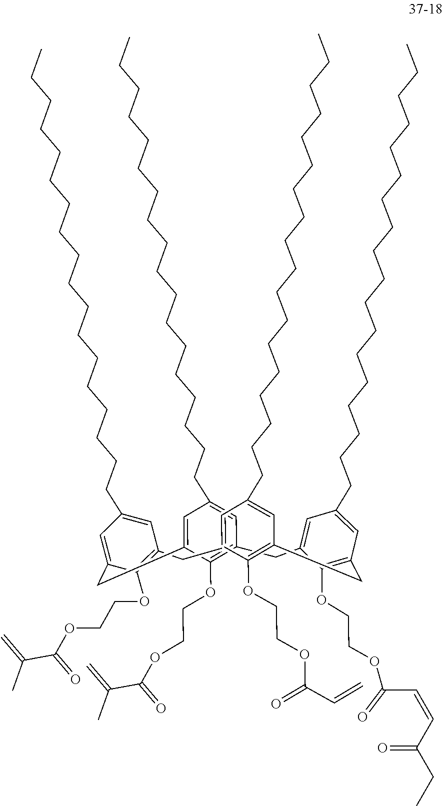

### Example 20

The same procedure as in Example 16 was repeated except that I-1 was used instead of I-6, to thereby obtain 0.381 g of the target compound 34-1 with a yield of 23.6%, 0.222 g of the target compound 35-1 with a yield of 14.3%, 0.231 g of the target compound 36-1 with a yield of 14.9%, and 0.129 g of the target compound 37-1 with a yield of 8.71%.

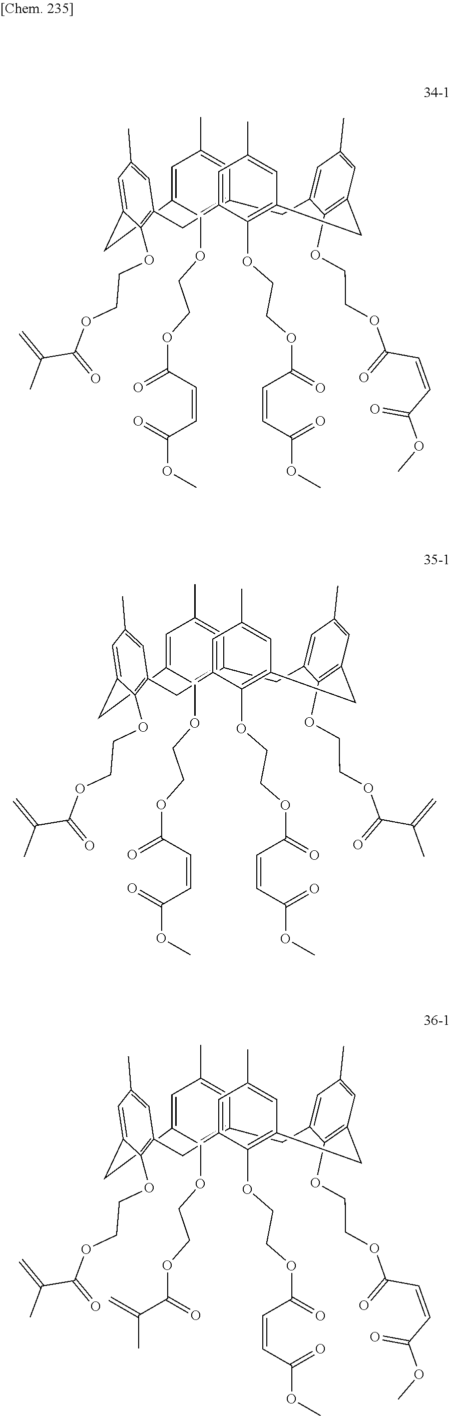

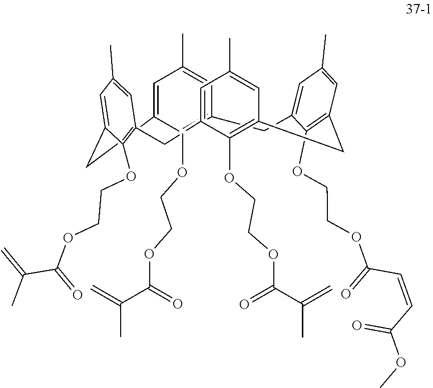 [SMILES: C=C(C)C(=O)OCCOc1c2cc(C)cc1Cc1cc(C)cc(c1OCCOC(=O)C(=C)C)Cc1cc(C)cc(c1OCCOC(=O)C(=C)C)Cc1cc(C)cc(c1OCCOC(=O)/C=C\C(=O)OC)C2]

### Example 21

The same procedure as in Example 16 was repeated except that acrylic acid was used instead of methacrylic acid, to thereby obtain 0.421 g of the target compound 38-6 with a yield of 29.7%, 0.237 g of the target compound 39-6 with a yield of 17.5%, 0.221 g of the target compound 40-6 with a yield of 16.3%, and 0.146 g of the target compound 41-6 with a yield of 11.3%.

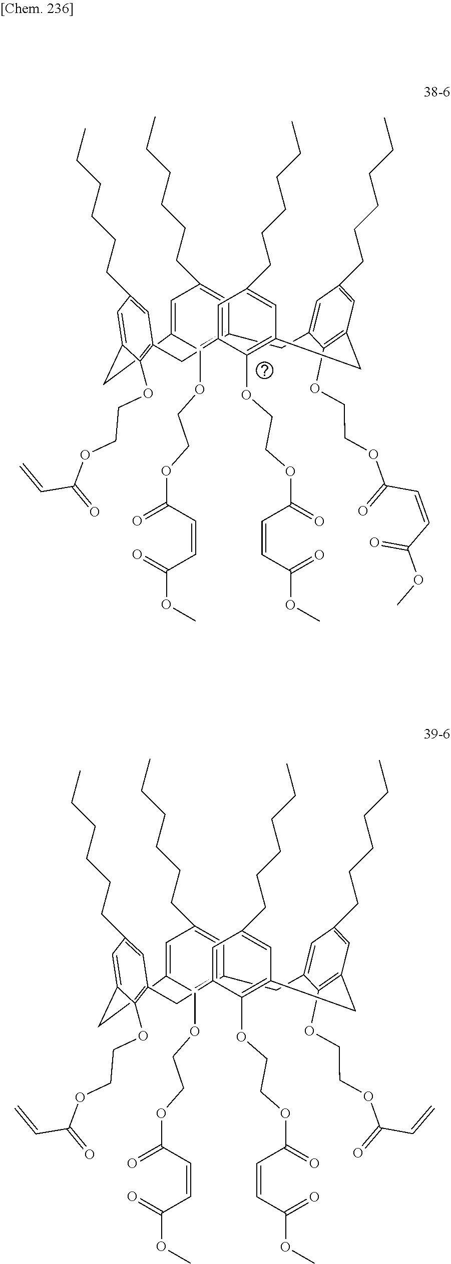 [SMILES: C=CC(=O)OCCOc1c2cc(CCCCCC)cc1Cc1cc(CCCCCC)cc(c1OCCOC(=O)/C=C\C(=O)OC)Cc1cc(CCCCCC)cc(c1OCCOC(=O)/C=C\C(=O)OC)Cc1cc(CCCCCC)cc(c1OCCOC(=O)/C=C\C(=O)OC)C2 ; C=CC(=O)OCCOc1c2cc(CCCCCC)cc1Cc1cc(CCCCCC)cc(c1OCCOC(=O)/C=C\C(=O)OC)Cc1cc(CCCCCC)cc(c1OCCOC(=O)C=C)Cc1cc(CCCCCC)cc(c1OCCOC(=O)/C=C\C(=O)OC)C2]

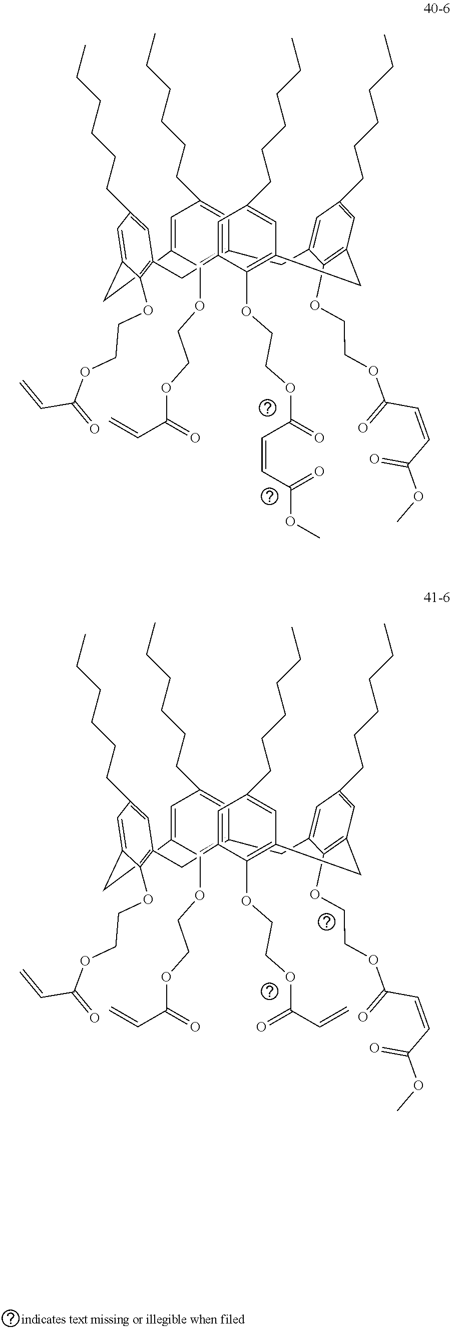 [SMILES: C=CC(=O)OCCOc1c2cc(CCCCCC)cc1Cc1cc(CCCCCC)cc(c1OCCOC(=O)/C=C\C(=O)OC)Cc1cc(CCCCCC)cc(c1OCCOC(=O)/C=C\C(=O)OC)Cc1cc(CCCCCC)cc(c1OCCOC(=O)C=C)C2 ; C=CC(=O)OCCOc1c2cc(CCCCCC)cc1Cc1cc(CCCCCC)cc(c1OCCOC(=O)C=C)Cc1cc(CCCCCC)cc(c1OCCOC(=O)/C=C\C(=O)OC)Cc1cc(CCCCCC)cc(c1OCCOC(=O)C=C)C2]

### Example 22

The same procedure as in Example 16 was repeated except that monoethyl maleate was used instead of monomethyl maleate, to thereby obtain 0.421 g of the target compound 42-6 with a yield of 28.5%, 0.237 g of the target compound 43-6 with a yield of 16.8%, 0.221 g of the target compound 44-6 with a yield of 15.6%, and 0.146 g of the target compound 45-6 with a yield of 10.8%.

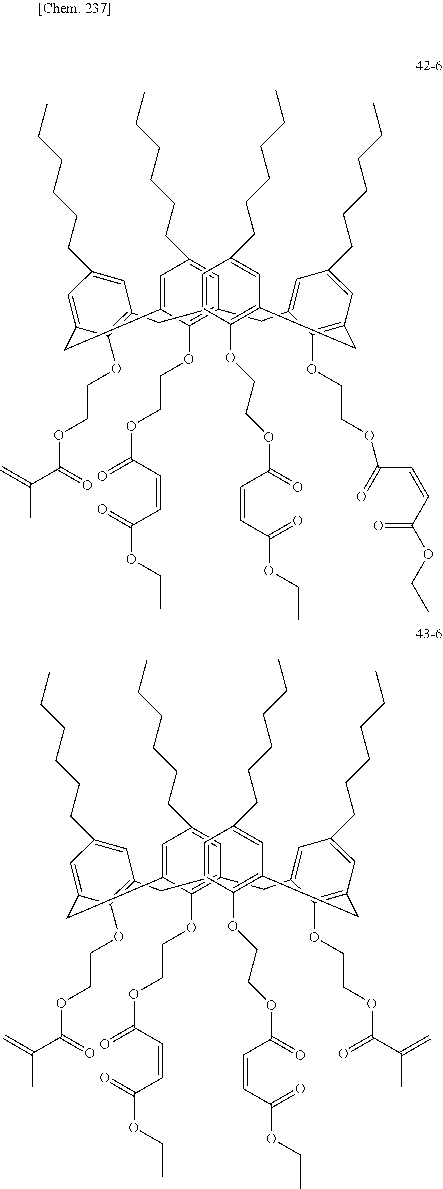

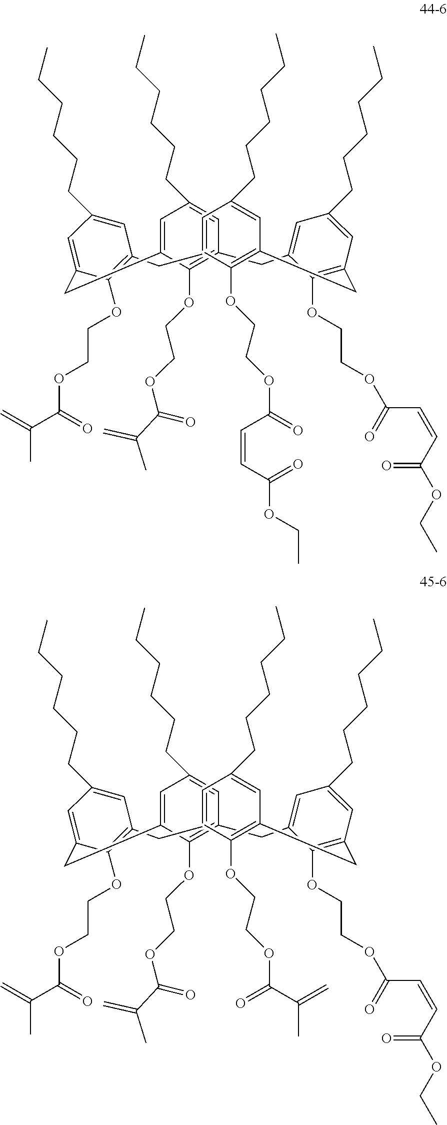

### Example 23

The same procedure as in Example 22 was repeated except that acrylic acid was used instead of methacrylic acid, to thereby obtain 0.418 g of the target compound 46-6 with a yield of 28.6%, 0.219 g of the target compound 47-6 with a yield of 15.8%, 0.207 g of the target compound 48-6 with a yield of 15.0%, and 0.138 g of the target compound 49-6 with a yield of 10.6%.

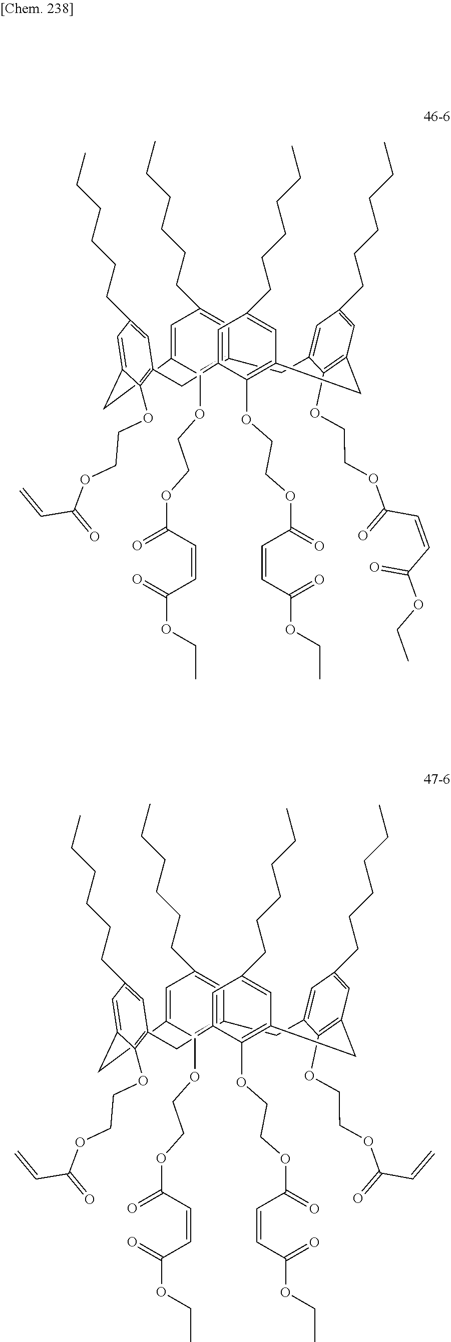 [SMILES: C=CC(=O)OCCOc1c2cc(CCCCCC)cc1Cc1cc(CCCCCC)cc(c1OCCOC(=O)/C=C\C(=O)OCC)Cc1cc(CCCCCC)cc(c1OCCOC(=O)/C=C\C(=O)OCC)Cc1cc(CCCCCC)cc(c1OCCOC(=O)/C=C\C(=O)OCC)C2 ; C=CC(=O)OCCOc1c2cc(CCCCCC)cc1Cc1cc(CCCCCC)cc(c1OCCOC(=O)/C=C\C(=O)OCC)Cc1cc(CCCCCC)cc(c1OCCOC(=O)C=C)Cc1cc(CCCCCC)cc(c1OCCOC(=O)/C=C\C(=O)OCC)C2]

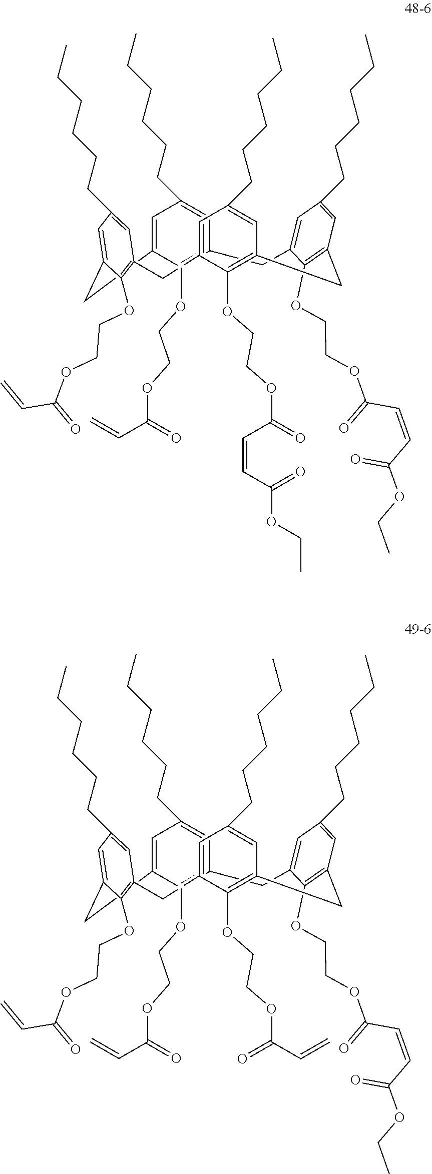 [SMILES: C=CC(=O)OCCOc1c2cc(CCCCCC)cc1Cc1cc(CCCCCC)cc(c1OCCOC(=O)/C=C\C(=O)OCC)Cc1cc(CCCCCC)cc(c1OCCOC(=O)/C=C\C(=O)OCC)Cc1cc(CCCCCC)cc(c1OCCOC(=O)C=C)C2 ; C=CC(=O)OCCOc1c2cc(CCCCCC)cc1Cc1cc(CCCCCC)cc(c1OCCOC(=O)C=C)Cc1cc(CCCCCC)cc(c1OCCOC(=O)/C=C\C(=O)OCC)Cc1cc(CCCCCC)cc(c1OCCOC(=O)C=C)C2]

### Synthesis Example 30

The same procedure as in Synthesis Example 20 was repeated except that methyl bromopyopionate was used instead of methyl bromoacetate, to thereby obtain 4.89 g of compound J-6 represented by the following structural formula. The yield was 67.3%.

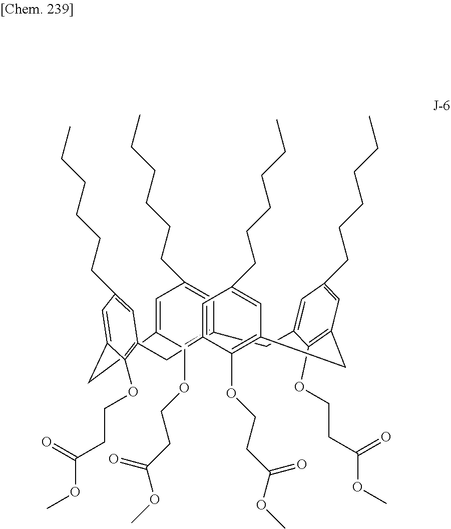 [SMILES: CCCCCCc1cc2c(OCCC(=O)OC)c(c1)Cc1cc(CCCCCC)cc(c1OCCC(=O)OC)Cc1cc(CCCCCC)cc(c1OCCC(=O)OC)Cc1cc(CCCCCC)cc(c1OCCC(=O)OC)C2]

### Synthesis Example 31

The same procedure as in Synthesis Example 10 was repeated except that J-6 was used instead of C-6, to thereby obtain 3.88 g of compound K-6 represented by the following structural formula. The yield was 88.3%.

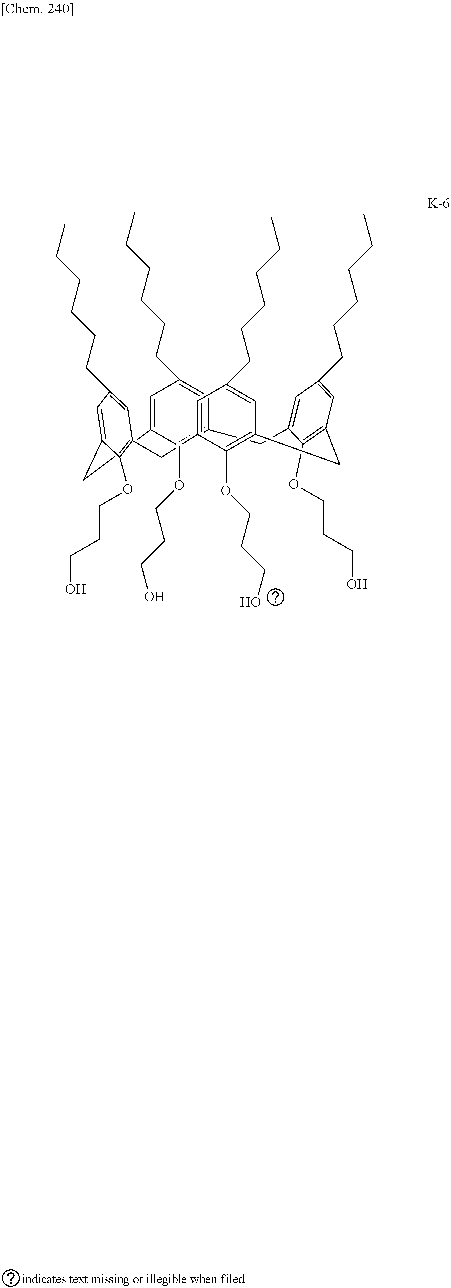 [SMILES: CCCCCCc1cc2c(OCCCO)c(c1)Cc1cc(CCCCCC)cc(c1OCCCO)Cc1cc(CCCCCC)cc(c1OCCCO)Cc1cc(CCCCCC)cc(c1OCCCO)C2]

### Example 24

The same procedure as in Example 1 was repeated except that K-6 was used instead of D-6, to thereby obtain 0.420 g of the target compound 50-6 with a yield of 29.9%, 0.208 g of the target compound 51-6 with a yield of 15.3%, 0.199 g of the target compound 52-6 with a yield of 14.6%, and 0.124 g of the target compound 53-6 with a yield of 9.41%.

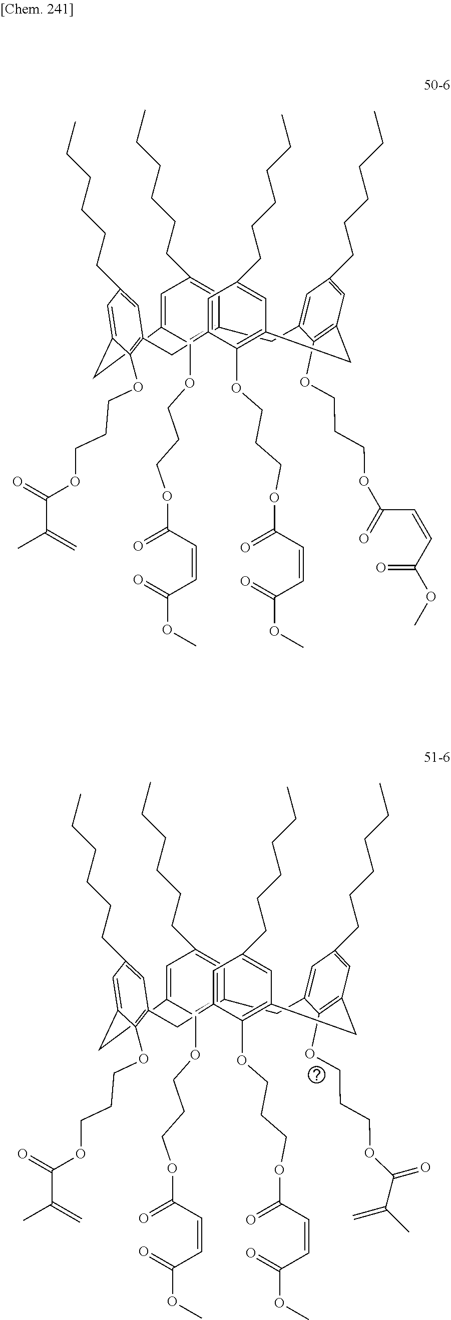 [SMILES: C=C(C)C(=O)OCCCOc1c2cc(CCCCCC)cc1Cc1cc(CCCCCC)cc(c1OCCCOC(=O)/C=C\C(=O)OC)Cc1cc(CCCCCC)cc(c1OCCCOC(=O)/C=C\C(=O)OC)Cc1cc(CCCCCC)cc(c1OCCCOC(=O)/C=C\C(=O)OC)C2 ; C=C(C)C(=O)OCCCOc1c2cc(CCCCCC)cc1Cc1cc(CCCCCC)cc(c1OCCCOC(=O)/C=C\C(=O)OC)Cc1cc(CCCCCC)cc(c1OCCCOC(=O)C(=C)C)Cc1cc(CCCCCC)cc(c1OCCCOC(=O)/C=C\C(=O)OC)C2]

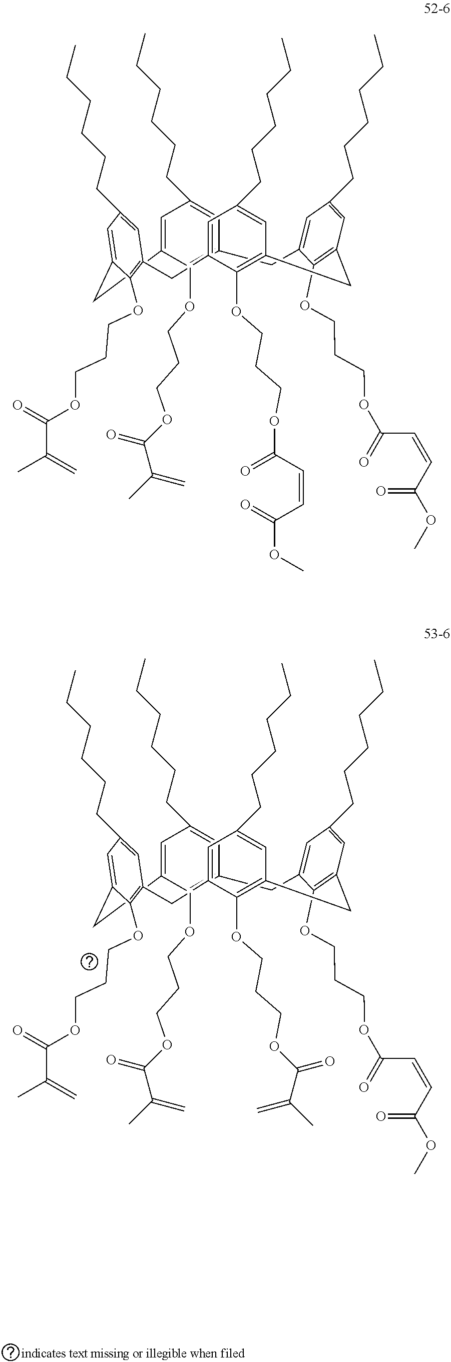 [SMILES: C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)/C=C\C(=O)OC)Cc1cc(CCCCCC)cc(c1OCCCOC(=O)/C=C\C(=O)OC)C2 ; C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)C(=C)C)Cc1cc(CCCCCC)cc(c1OCCCOC(=O)/C=C\C(=O)OC)C2]

### Example 25

The same procedure as in Example 21 was repeated except that acrylic acid was used instead of methacrylic acid, to thereby obtain 0.399 g of the target compound 54-6 with a yield of 28.6%, 0.212 g of the target compound 55-6 with a yield of 15.9%, 0.219 g of the target compound 56-6 with a yield of 16.4%, and 0.134 g of the target compound 57-6 with a yield of 10.1%.

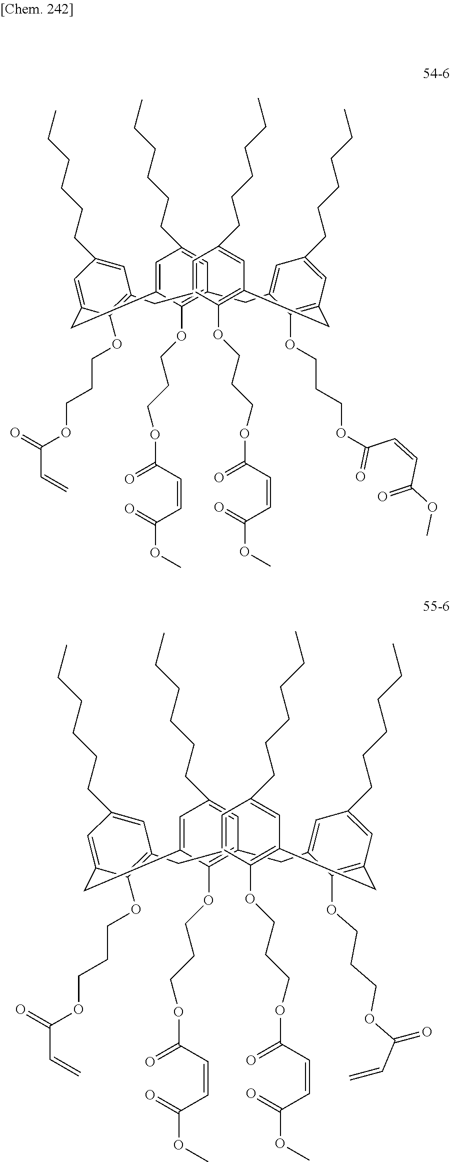

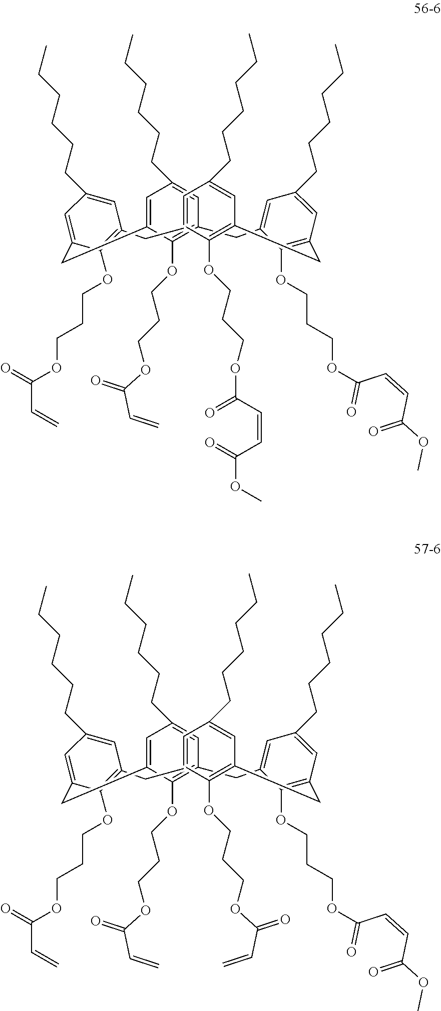

### Example 26

The same procedure as in Example 21 was repeated except that monoethyl maleate was used instead of monomethyl maleate, to thereby obtain 0.421 g of the target compound 58-6 with a yield of 29.0%, 0.222 g of the target compound 59-6 with a yield of 16.0%, 0.217 g of the target compound 60-6 with a yield of 15.6%, and 0.141 g of the target compound 61-6 with a yield of 10.6%.

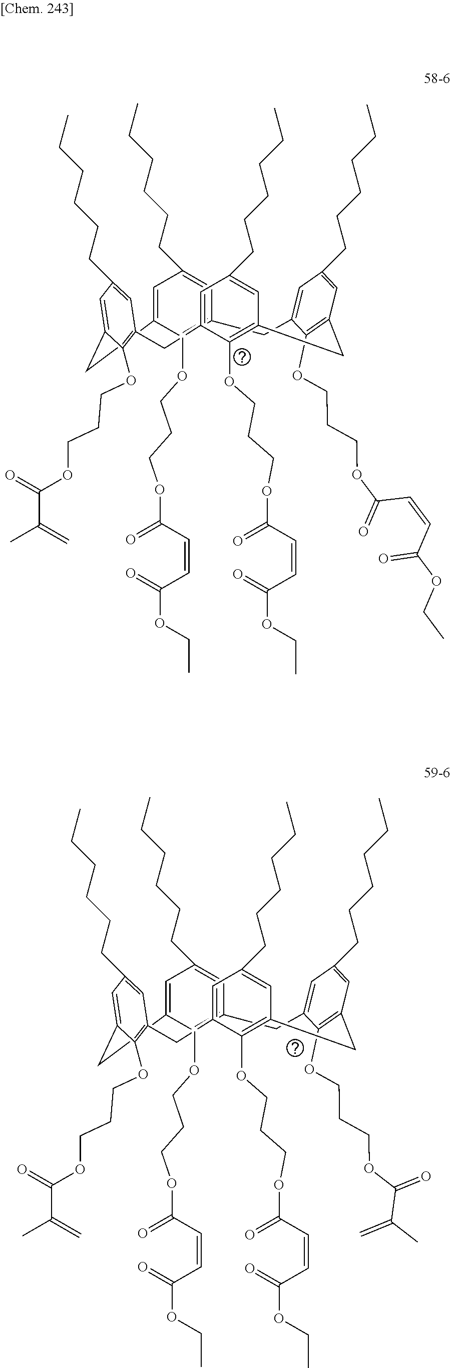 [SMILES: C=C(C)C(=O)OCCCOc1c2cc(CCCCCC)cc1Cc1cc(CCCCCC)cc(c1OCCCOC(=O)/C=C\C(=O)OCC)Cc1cc(CCCCCC)cc(c1OCCCOC(=O)/C=C\C(=O)OCC)Cc1cc(CCCCCC)cc(c1OCCCOC(=O)/C=C\C(=O)OCC)C2 ; C=C(C)C(=O)OCCCOc1c2cc(CCCCCC)cc1Cc1cc(CCCCCC)cc(c1OCCCOC(=O)/C=C\C(=O)OCC)Cc1cc(CCCCCC)cc(c1OCCCOC(=O)C(=C)C)Cc1cc(CCCCCC)cc(c1OCCCOC(=O)/C=C\C(=O)OCC)C2]

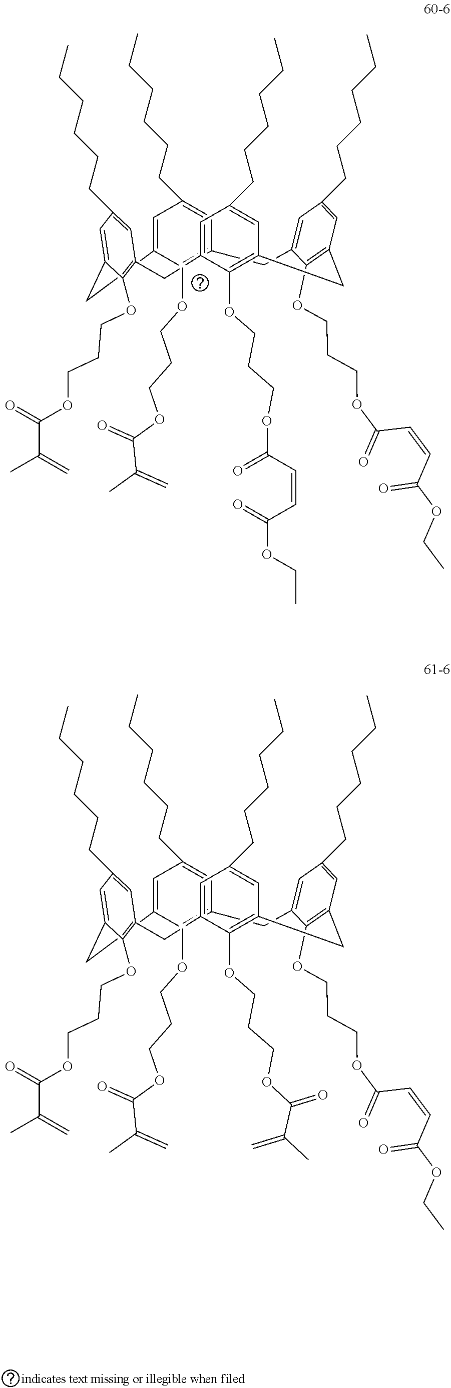 [SMILES: C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)/C=C\C(=O)OCC)Cc1cc(CCCCCC)cc(c1OCCCOC(=O)/C=C\C(=O)OCC)C2 ; C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)C(=C)C)Cc1cc(CCCCCC)cc(c1OCCCOC(=O)/C=C\C(=O)OCC)C2]

### Example 27

The same procedure as in Example 23 was repeated except that acrylic acid was used instead of methacrylic acid, to thereby obtain 0.408 g of the target compound 62-6 with a yield of 28.4%, 0.21 g of the target compound 63-6 with a yield of 15.4%, 0.206 g of the target compound 64-6 with a yield of 15.1%, and 0.127 g of the target compound 65-6 with a yield of 9.84%.

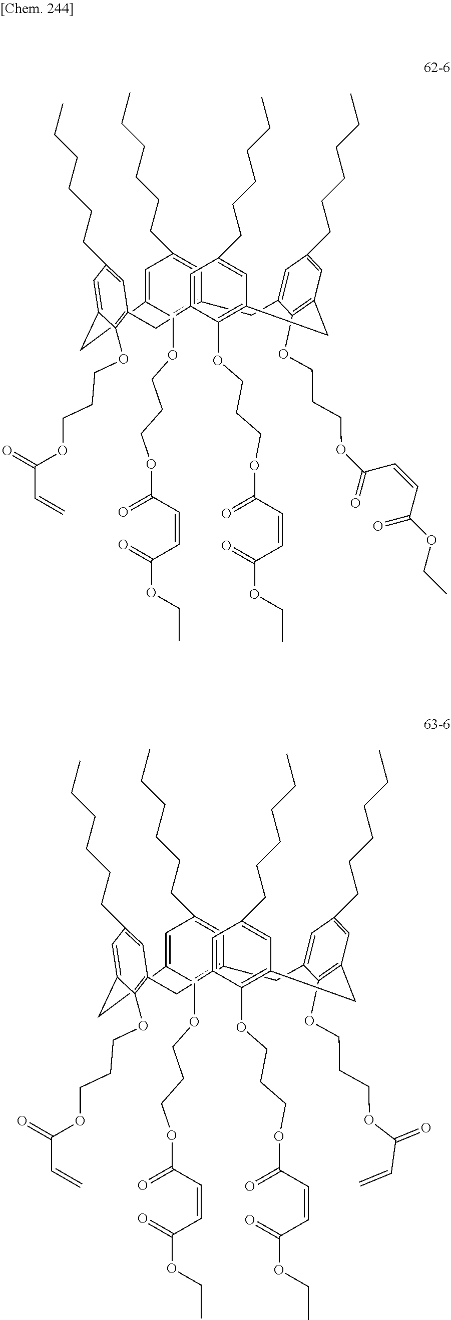 [SMILES: C=CC(=O)OCCCOc1c2cc(CCCCCC)cc1Cc1cc(CCCCCC)cc(c1OCCCOC(=O)/C=C\C(=O)OCC)Cc1cc(CCCCCC)cc(c1OCCCOC(=O)/C=C\C(=O)OCC)Cc1cc(CCCCCC)cc(c1OCCCOC(=O)/C=C\C(=O)OCC)C2 ; C=CC(=O)OCCCOc1c2cc(CCCCCC)cc1Cc1cc(CCCCCC)cc(c1OCCCOC(=O)/C=C\C(=O)OCC)Cc1cc(CCCCCC)cc(c1OCCCOC(=O)C=C)Cc1cc(CCCCCC)cc(c1OCCCOC(=O)/C=C\C(=O)OCC)C2]

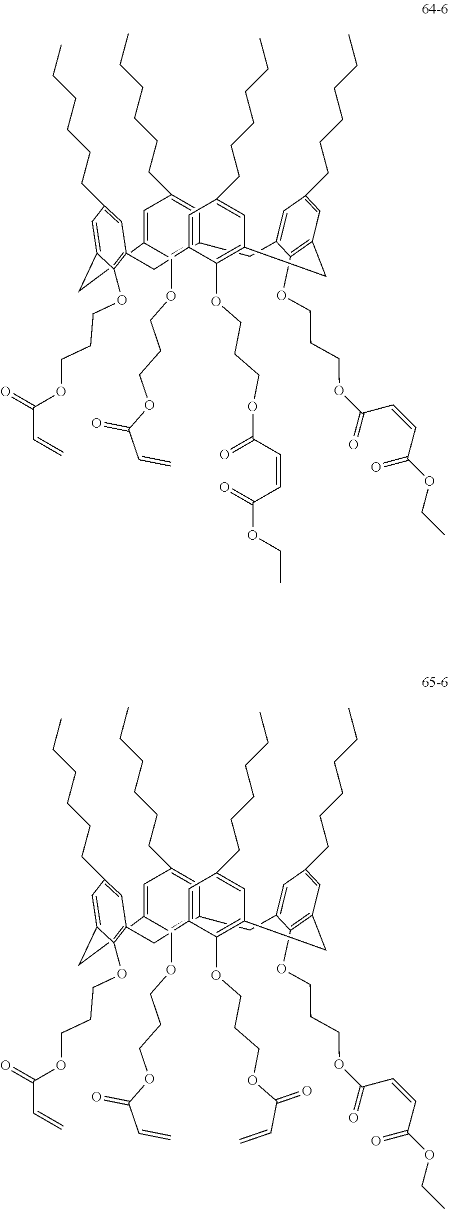 [SMILES: C=CC(=O)OCCCOc1c2cc(CCCCCC)cc1Cc1cc(CCCCCC)cc(c1OCCCOC(=O)/C=C\C(=O)OCC)Cc1cc(CCCCCC)cc(c1OCCCOC(=O)/C=C\C(=O)OCC)Cc1cc(CCCCCC)cc(c1OCCCOC(=O)C=C)C2 ; C=CC(=O)OCCCOc1c2cc(CCCCCC)cc1Cc1cc(CCCCCC)cc(c1OCCCOC(=O)C=C)Cc1cc(CCCCCC)cc(c1OCCCOC(=O)/C=C\C(=O)OCC)Cc1cc(CCCCCC)cc(c1OCCCOC(=O)C=C)C2]

### Synthesis Example 32

A 50 mL four-necked flask equipped with a stirrer, a thermometer, and a reflux condenser tube was charged with 2.00 g (2.424 mmol) of I-6, 10.00 g of tetrahydrofuran, 1.2716 g (4.848 mmol) of triphenylphosphine, and 1.024 g (4.732 mmol) of 2-[[[(1,1-dimethylethyl)dimethylsilyl]oxy]-2-propenoic acid, and the mixture was stirred. A light yellow transparent solution was thereby obtained. Next, 0.9803 g (4.848 mmol) of diisopropyl azodicarboxylate was added dropwise to the light yellow transparent solution placed in an ice bath over 30 minutes. The light yellow transparent solution remained unchanged. The solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:acetone=95:5) to thereby obtain a light yellow transparent liquid. The solvent was concentrated, and chloroform/methanol were added to reprecipitate crystals. The white crystals were filtered on a Kiriyama funnel, and the obtained white crystals were vacuum dried (at 60° C. for 6 hours or longer) to thereby obtain 1.891 g of compound M-6 represented by the following structural formula. The yield was 48.2%.

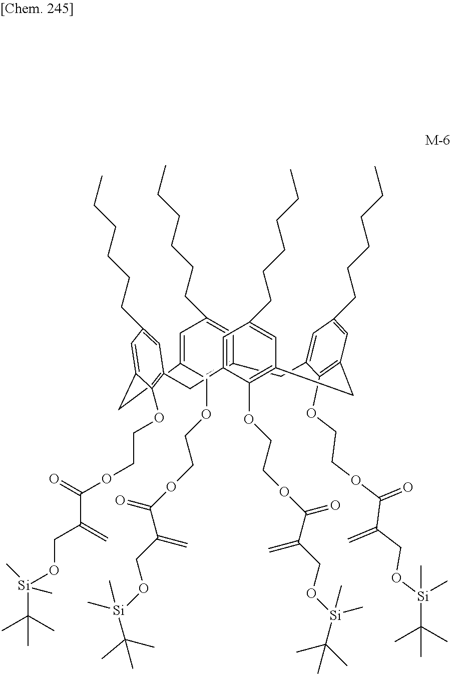 [SMILES: C=C(CO[Si](C)(C)C(C)(C)C)C(=O)OCCOc1c2cc(CCCCCC)cc1Cc1cc(CCCCCC)cc(c1OCCOC(=O)C(=C)CO[Si](C)(C)C(C)(C)C)Cc1cc(CCCCCC)cc(c1OCCOC(=O)C(=C)CO[Si](C)(C)C(C)(C)C)Cc1cc(CCCCCC)cc(c1OCCOC(=O)C(=C)CO[Si](C)(C)C(C)(C)C)C2]

### Synthesis Example 33

The same procedure as in Synthesis Example 32 was repeated except that I-4 was used instead of I-6, to thereby obtain 1.641 g of compound M-4 represented by the following structural formula. The yield was 57.3%.

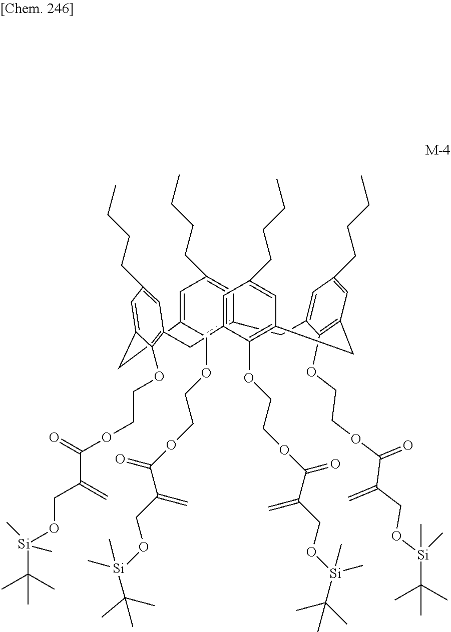 [SMILES: C=C(CO[Si](C)(C)C(C)(C)C)C(=O)OCCOc1c2cc(CCCC)cc1Cc1cc(CCCC)cc(c1OCCOC(=O)C(=C)CO[Si](C)(C)C(C)(C)C)Cc1cc(CCCC)cc(c1OCCOC(=O)C(=C)CO[Si](C)(C)C(C)(C)C)Cc1cc(CCCC)cc(c1OCCOC(=O)C(=C)CO[Si](C)(C)C(C)(C)C)C2]

### Synthesis Example 34

The same procedure as in Synthesis Example 32 was repeated except that I-7 was used instead of I-6, to thereby obtain 1.880 g of compound M-7 represented by the following structural formula. The yield was 79.0%.

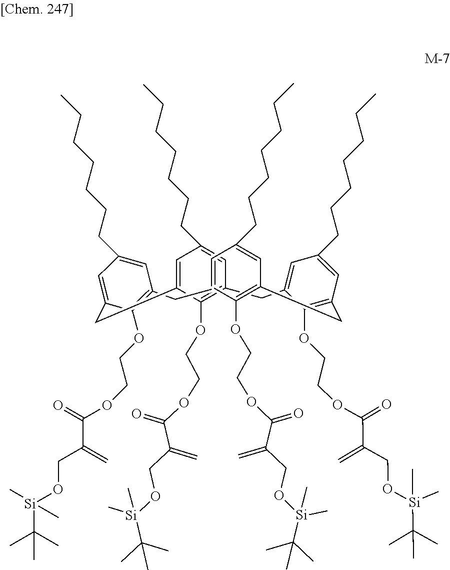

### Synthesis Example 35

The same procedure as in Synthesis Example 32 was repeated except that I-18 was used instead of I-6, to thereby obtain 2.132 g of compound M-18 represented by the following structural formula. The yield was 71.4%.

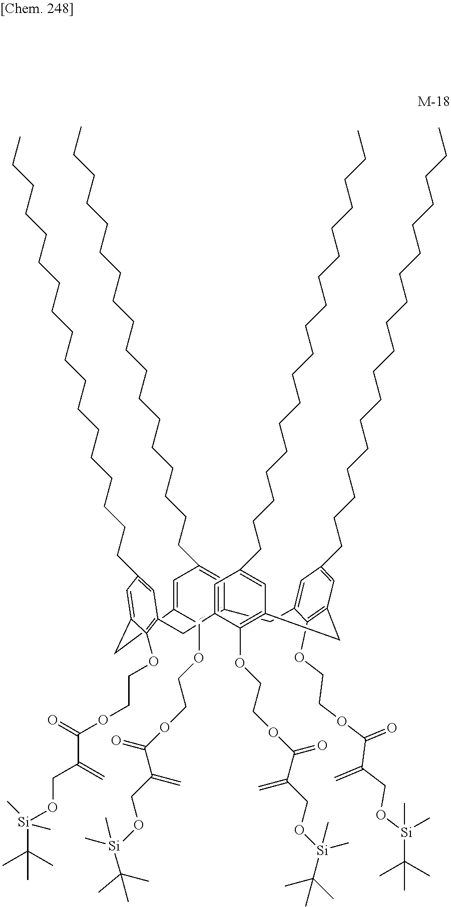 [SMILES: C=C(CO[Si](C)(C)C(C)(C)C)C(=O)OCCOc1c2cc(CCCCCCCCCCCCCCCCCC)cc1Cc1cc(CCCCCCCCCCCCCCCCCC)cc(c1OCCOC(=O)C(=C)CO[Si](C)(C)C(C)(C)C)Cc1cc(CCCCCCCCCCCCCCCCCC)cc(c1OCCOC(=O)C(=C)CO[Si](C)(C)C(C)(C)C)Cc1cc(CCCCCCCCCCCCCCCCCC)cc(c1OCCOC(=O)C(=C)CO[Si](C)(C)C(C)(C)C)C2]

### Synthesis Example 36

The same procedure as in Synthesis Example 32 was repeated except that I-1 was used instead of I-6, to thereby obtain 1.762 g of compound M-1 represented by the following structural formula. The yield was 39.9%.

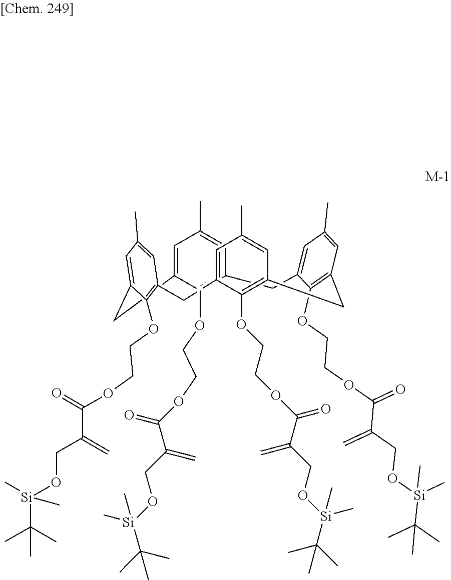 [SMILES: C=C(CO[Si](C)(C)C(C)(C)C)C(=O)OCCOc1c2cc(C)cc1Cc1cc(C)cc(c1OCCOC(=O)C(=C)CO[Si](C)(C)C(C)(C)C)Cc1cc(C)cc(c1OCCOC(=O)C(=C)CO[Si](C)(C)C(C)(C)C)Cc1cc(C)cc(c1OCCOC(=O)C(=C)CO[Si](C)(C)C(C)(C)C)C2]

### Synthesis Example 37

A 100 mL four-necked flask equipped with a stirrer, a thermometer, and a reflux condenser tube was charged with 1.891 g (1.168 mmol) of M-6, 50.00 g of tetrahydrofuran, and 0.3367 g (5.606 mmol) of acetic acid, and the mixture was stirred. Then tetrabutylammonium fluoride (an about 1 mol/L tetrahydrofuran solution; 5.61 mL (5.61 mmol)) was slowly added dropwise to the mixture placed in an ice bath under stirring. The resulting light yellow transparent reaction solution was stirred at room temperature for 6 hours. Ion exchanged water was added to the reaction solution placed in an ice bath to stop the reaction, and 30 g of chloroform was added. The reaction mixture was transferred to a separatory funnel, and the organic layer was separated. Next, the aqueous layer was extracted with 30 g of chloroform three times, and the extract was combined with the organic layer. The organic layer was pre-dried over anhydrous magnesium sulfate, and the solvent was removed by evaporation using an evaporator to thereby obtain a red transparent liquid. The red transparent liquid was purified by column chromatography (eluent: n-hexane:acetone=95:5), and chloroform/methanol were added to the obtained light yellow transparent liquid to reprecipitate crystals. The white crystals were filtered on a Kiriyama funnel and vacuum dried (at 60° C. for 6 hours or longer) to thereby obtain 0.8451 g of compound N-6 represented by the following structural formula. The yield was 62.3%.

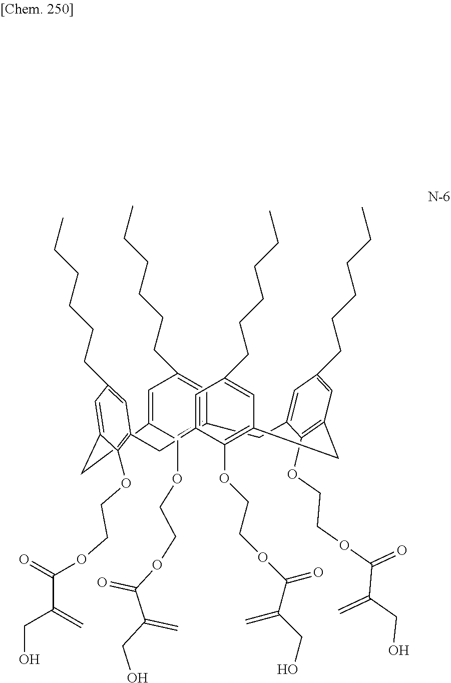 [SMILES: C=C(CO)C(=O)OCCOc1c2cc(CCCCCC)cc1Cc1cc(CCCCCC)cc(c1OCCOC(=O)C(=C)CO)Cc1cc(CCCCCC)cc(c1OCCOC(=O)C(=C)CO)Cc1cc(CCCCCC)cc(c1OCCOC(=O)C(=C)CO)C2]

### Synthesis Example 38

The same procedure as in Synthesis Example 37 was repeated except that M-4 was used instead of M-6, to thereby obtain 0.639 g of compound N-4 represented by the following structural formula. The yield was 54.3%.

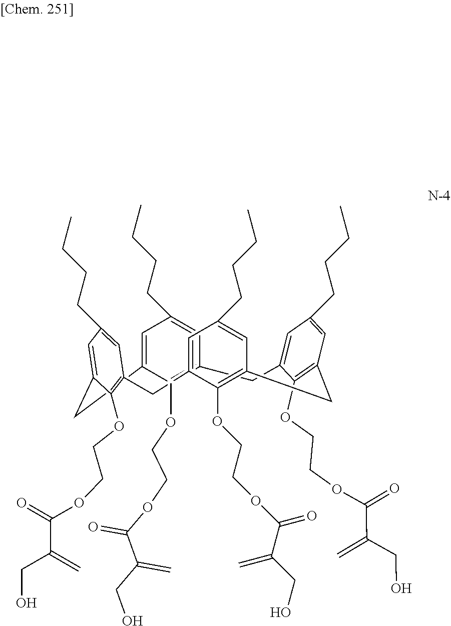 [SMILES: C=C(CO)C(=O)OCCOc1c2cc(CCCC)cc1Cc1cc(CCCC)cc(c1OCCOC(=O)C(=C)CO)Cc1cc(CCCC)cc(c1OCCOC(=O)C(=C)CO)Cc1cc(CCCC)cc(c1OCCOC(=O)C(=C)CO)C2]

### Synthesis Example 39

The same procedure as in Synthesis Example 37 was repeated except that M-7 was used instead of M-6, to thereby obtain 0.873 g of compound N-7 represented by the following structural formula. The yield was 62.4%.

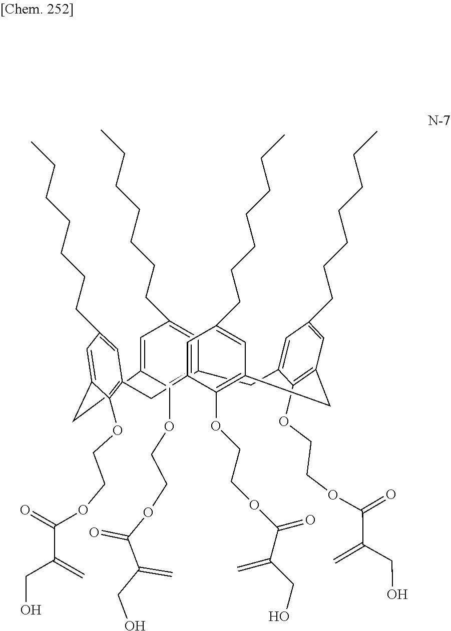 [SMILES: C=C(CO)C(=O)OCCOc1c2cc(CCCCCCC)cc1Cc1cc(CCCCCCC)cc(c1OCCOC(=O)C(=C)CO)Cc1cc(CCCCCCC)cc(c1OCCOC(=O)C(=C)CO)Cc1cc(CCCCCCC)cc(c1OCCOC(=O)C(=C)CO)C2]

### Synthesis Example 40

The same procedure as in Synthesis Example 37 was repeated except that M-18 was used instead of M-6, to thereby obtain 1.092 g of compound N-18 represented by the following structural formula. The yield was 63.2%.

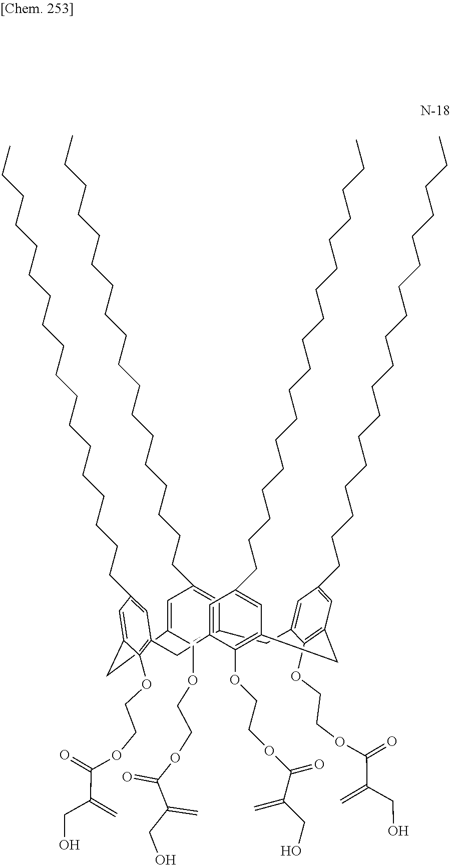 [SMILES: C=C(CO)C(=O)OCCOc1c2cc(CCCCCCCCCCCCCCCCCC)cc1Cc1cc(CCCCCCCCCCCCCCCCCC)cc(c1OCCOC(=O)C(=C)CO)Cc1cc(CCCCCCCCCCCCCCCCCC)cc(c1OCCOC(=O)C(=C)CO)Cc1cc(CCCCCCCCCCCCCCCCCC)cc(c1OCCOC(=O)C(=C)CO)C2]

### Synthesis Example 41

The same procedure as in Synthesis Example 37 was repeated except that M-1 was used instead of M-6, to thereby obtain 0.654 g of compound N-1 represented by the following structural formula. The yield was 54.2%.

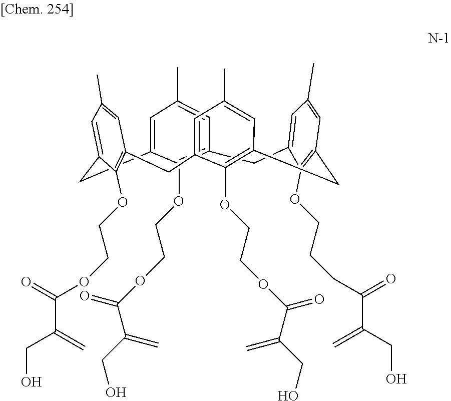 [SMILES: C=C(CO)C(=O)CCCOc1c2cc(C)cc1Cc1cc(C)cc(c1OCCOC(=O)C(=C)CO)Cc1cc(C)cc(c1OCCOC(=O)C(=C)CO)Cc1cc(C)cc(c1OCCOC(=O)C(=C)CO)C2]

### Example 28

A 30 mL four-necked flask equipped with a stirrer, a thermometer, and a reflux condenser tube was charged with 0.300 g (0.236 mmol) of N-6, 0.679 g of tetrahydrofuran, 0.494 g (1.884 mmol) of triphenylphosphine, and 0.245 g (1.884 mmol) monomethyl maleate, and the mixture was stirred. Then 0.423 g (1.884 mmol) of diisopropyl azodicarboxylate diluted with 0.340 g of tetrahydrofuran was added dropwise to the mixture placed in an ice bath over 30 minutes. The resulting light yellow transparent reaction solution was stirred at room temperature for 6 hours. Hexane was added to the reaction solution to precipitate and remove by-products such as triphenylphosphine, and the resulting solution was extracted with chloroform. The resulting solution was washed with water and saturated brine and dried over magnesium sulfate, and the solvent was removed by evaporation using an evaporator to thereby obtain a red viscous liquid. The red viscous liquid was purified by column chromatography (eluent: n-hexane:ethyl acetate=85:15) to thereby obtain 0.321 g of the target compound 66-6. The yield was 79.1%.

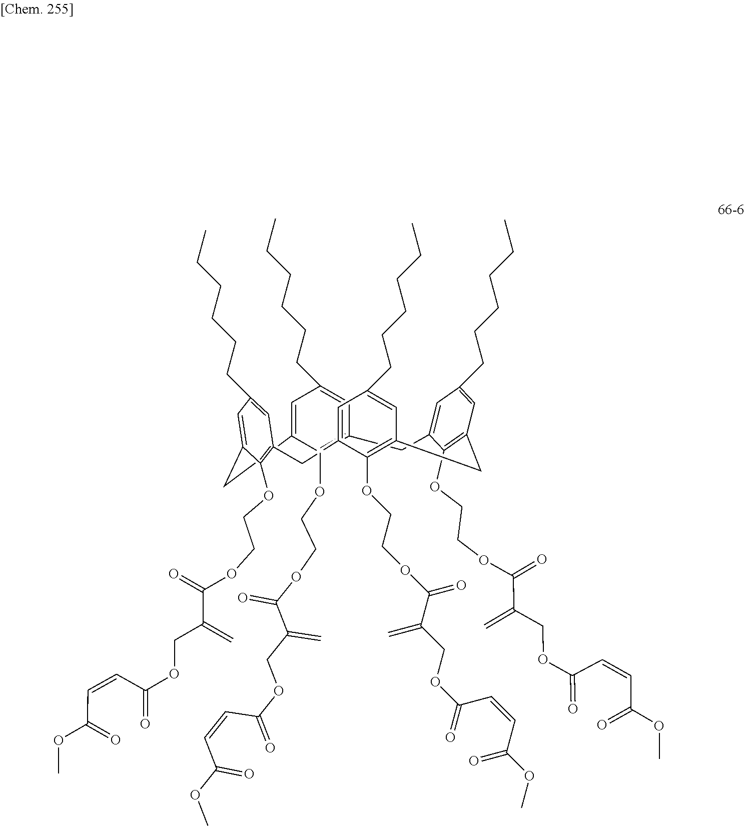

### Example 29

The same procedure as in Example 28 was repeated except that N-4 was used instead of N-6, to thereby obtain 0.306 g of the target compound 66-4. The yield was 73.6%.

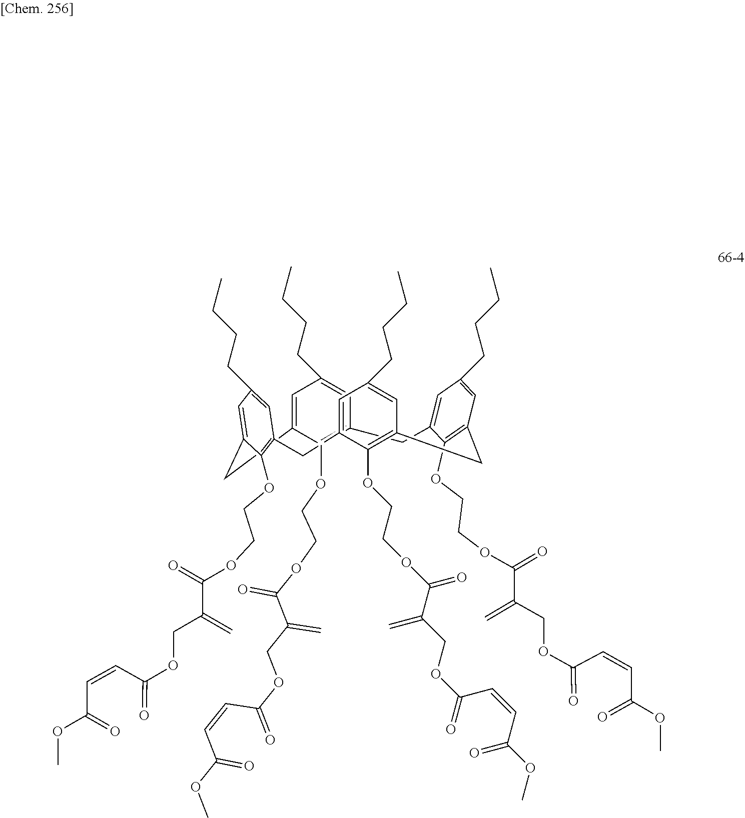

### Example 30

The same procedure as in Example 28 was repeated except that N-7 was used instead of N-6, to thereby obtain 0.323 g of the target compound 66-7. The yield was 77.1%.

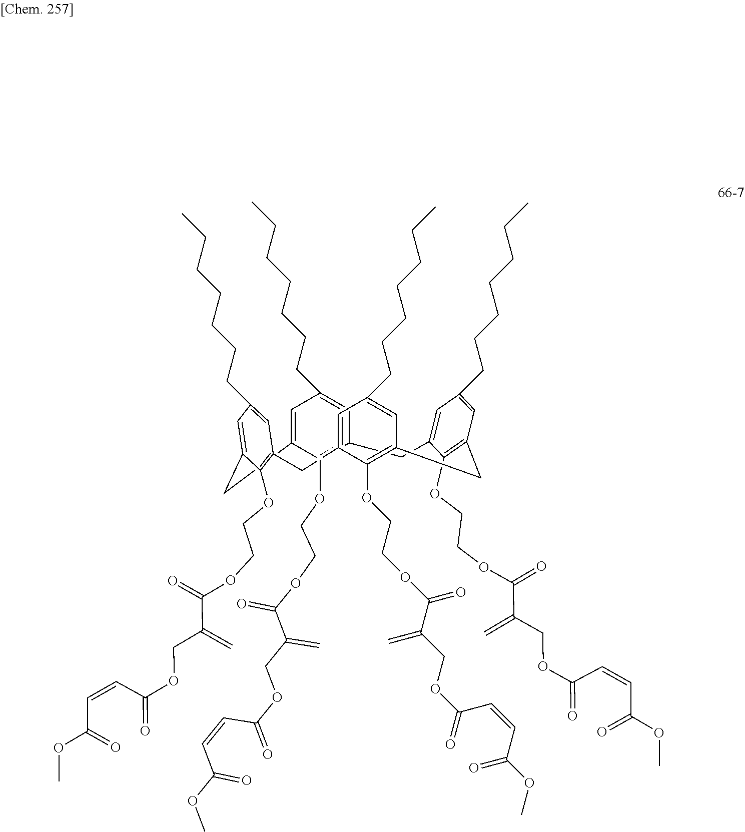 [SMILES: C=C(COC(=O)/C=C\C(=O)OC)C(=O)OCCOc1c2cc(CCCCCCC)cc1Cc1cc(CCCCCCC)cc(c1OCCOC(=O)C(=C)COC(=O)/C=C\C(=O)OC)Cc1cc(CCCCCCC)cc(c1OCCOC(=O)C(=C)COC(=O)/C=C\C(=O)OC)Cc1cc(CCCCCCC)cc(c1OCCOC(=O)C(=C)COC(=O)/C=C\C(=O)OC)C2]

### Example 31

The same procedure as in Example 28 was repeated except that N-18 was used instead of N-6, to thereby obtain 0.287 g of the target compound 66-18. The yield was 77.7%.

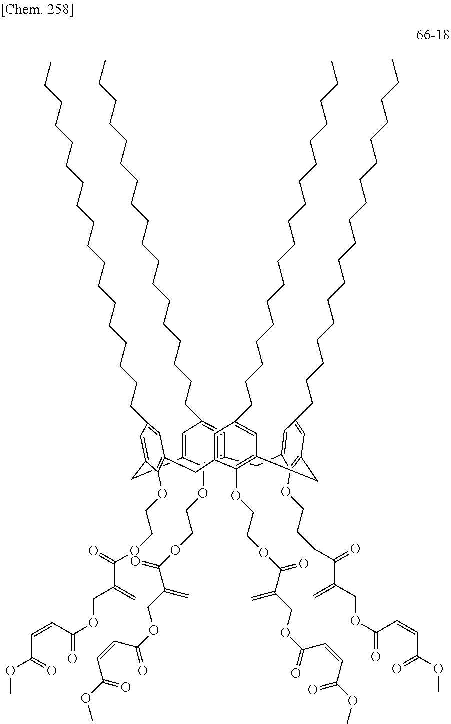 [SMILES: C=C(COC(=O)/C=C\C(=O)OC)C(=O)CCCOc1c2cc(CCCCCCCCCCCCCCCCCC)cc1Cc1cc(CCCCCCCCCCCCCCCCCC)cc(c1OCCOC(=O)C(=C)COC(=O)/C=C\C(=O)OC)Cc1cc(CCCCCCCCCCCCCCCCCC)cc(c1OCCOC(=O)C(=C)COC(=O)/C=C\C(=O)OC)Cc1cc(CCCCCCCCCCCCCCCCCC)cc(c1OCCOC(=O)C(=C)COC(=O)/C=C\C(=O)OC)C2]

### Example 32

The same procedure as in Example 28 was repeated except that N-1 was used instead of N-6, to thereby obtain 0.237 g of the target compound 66-1. The yield was 54.4%.

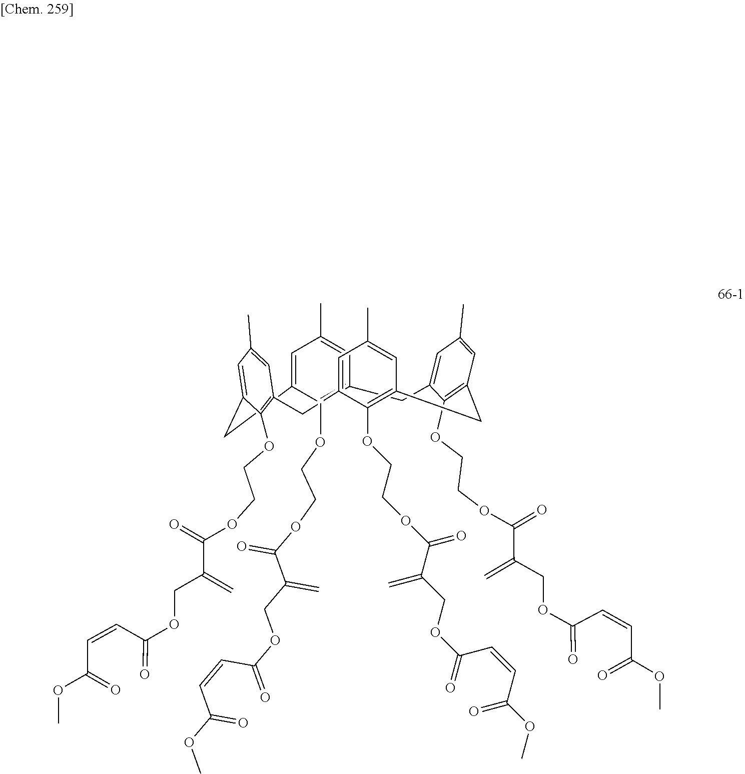

### Example 33

The same procedure as in Example 28 was repeated except that monoethyl maleate was used instead of monomethyl maleate, to thereby obtain 0.301 g of the target compound 67-6. The yield was 71.9%.

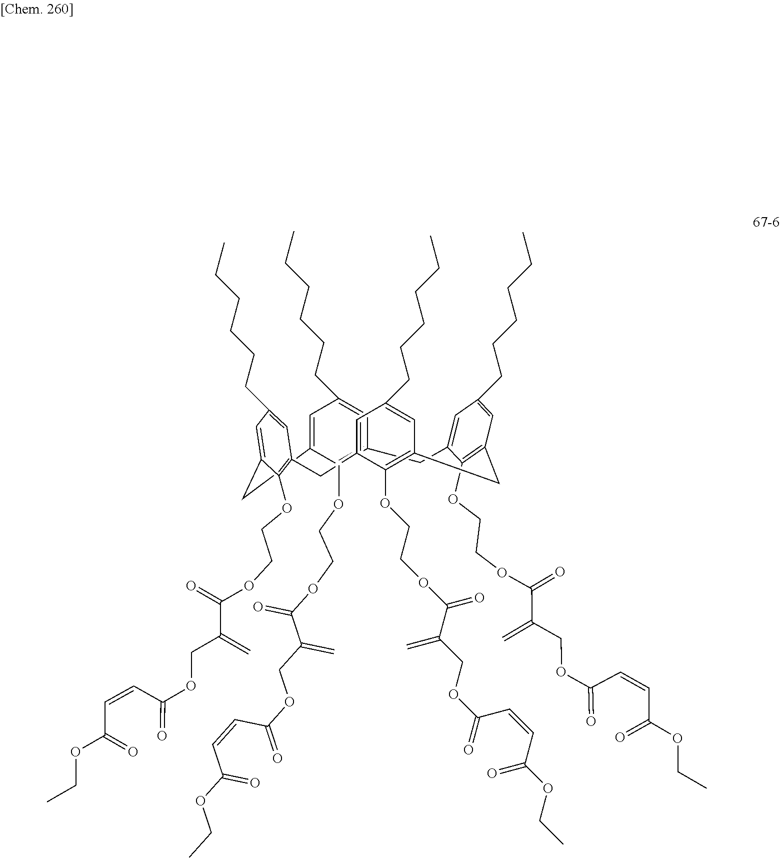 [SMILES: C=C(COC(=O)/C=C\C(=O)OCC)C(=O)OCCOc1c2cc(CCCCCC)cc1Cc1cc(CCCCCC)cc(c1OCCOC(=O)C(=C)COC(=O)/C=C\C(=O)OCC)Cc1cc(CCCCCC)cc(c1OCCOC(=O)C(=C)COC(=O)/C=C\C(=O)OCC)Cc1cc(CCCCCC)cc(c1OCCOC(=O)C(=C)COC(=O)/C=C\C(=O)OCC)C2]

### Synthesis Example 42

The same procedure as in Synthesis Example 32 was repeated except that 4-[[[(1,1-dimethylethyl)dimethylsilyl]oxy]-2-methylenebutanoic acid was used instead of 2-[[[(1,1-dimethylethyl)dimethylsilyl]oxy]2-propenoic acid, to thereby obtain 2.420 g of compound O-6 represented by the following structural formula. The yield was 72.6%.

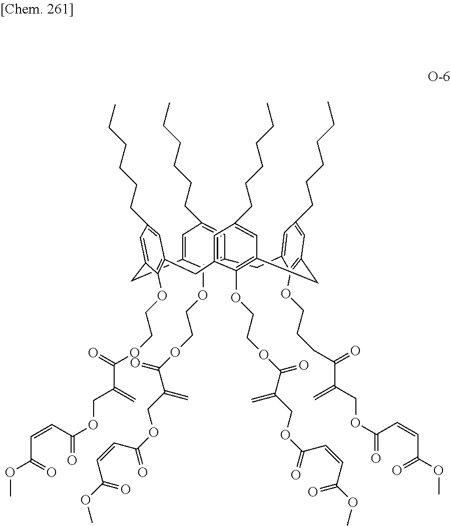 [SMILES: C=C(COC(=O)/C=C\C(=O)OC)C(=O)CCCOc1c2cc(CCCCCC)cc1Cc1cc(CCCCCC)cc(c1OCCOC(=O)C(=C)COC(=O)/C=C\C(=O)OC)Cc1cc(CCCCCC)cc(c1OCCOC(=O)C(=C)COC(=O)/C=C\C(=O)OC)Cc1cc(CCCCCC)cc(c1OCCOC(=O)C(=C)COC(=O)/C=C\C(=O)OC)C2]

### Synthesis Example 43

The same procedure as in Synthesis Example 37 was repeated except that O-6 was used instead of M-6, to thereby obtain 1.07 g of compound P-6 represented by the following structural formula. The yield was 59.4%.

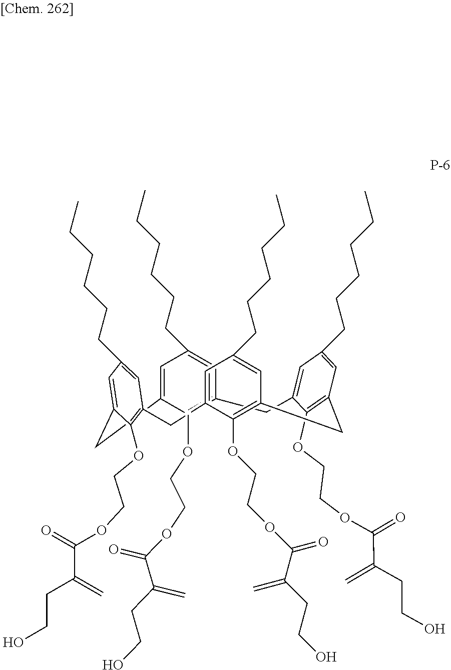 [SMILES: C=C(CCO)C(=O)OCCOc1c2cc(CCCCCC)cc1Cc1cc(CCCCCC)cc(c1OCCOC(=O)C(=C)CCO)Cc1cc(CCCCCC)cc(c1OCCOC(=O)C(=C)CCO)Cc1cc(CCCCCC)cc(c1OCCOC(=O)C(=C)CCO)C2]

### Example 34

The same procedure as in Example 28 was repeated except that P-6 was used instead of N-6, to thereby obtain 0.297 g of the target compound 68-6. The yield was 74.0%.

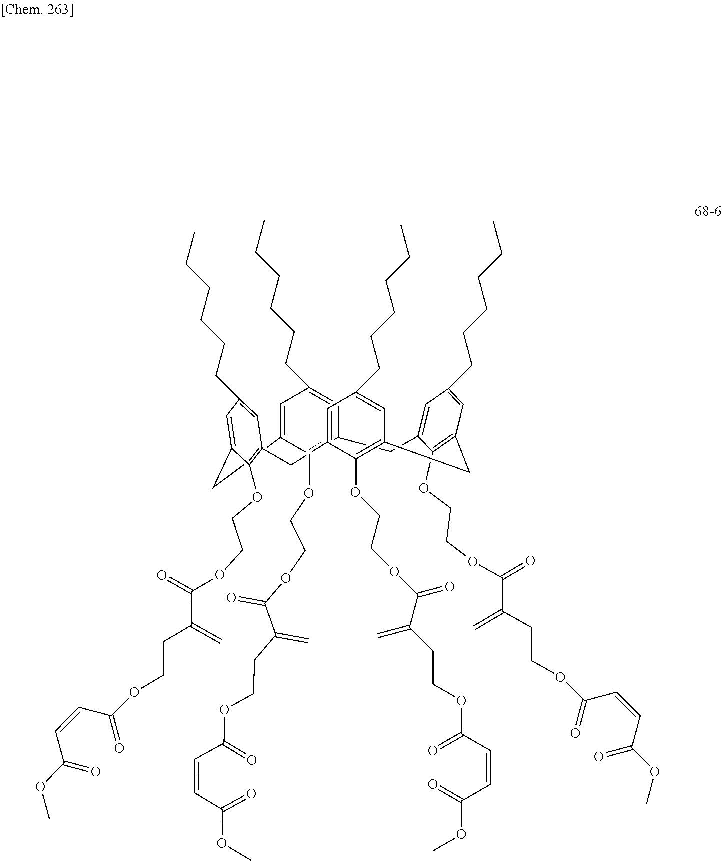 [SMILES: C=C(CCOC(=O)/C=C\C(=O)OC)C(=O)OCCOc1c2cc(CCCCCC)cc1Cc1cc(CCCCCC)cc(c1OCCOC(=O)C(=C)CCOC(=O)/C=C\C(=O)OC)Cc1cc(CCCCCC)cc(c1OCCOC(=O)C(=C)CCOC(=O)/C=C\C(=O)OC)Cc1cc(CCCCCC)cc(c1OCCOC(=O)C(=C)CCOC(=O)/C=C\C(=O)OC)C2]

### Example 35

The same procedure as in Example 34 was repeated except that monoethyl maleate was used instead of monomethyl maleate, to thereby obtain 0.277 g of the target compound 69-6. The yield was 66.9%.

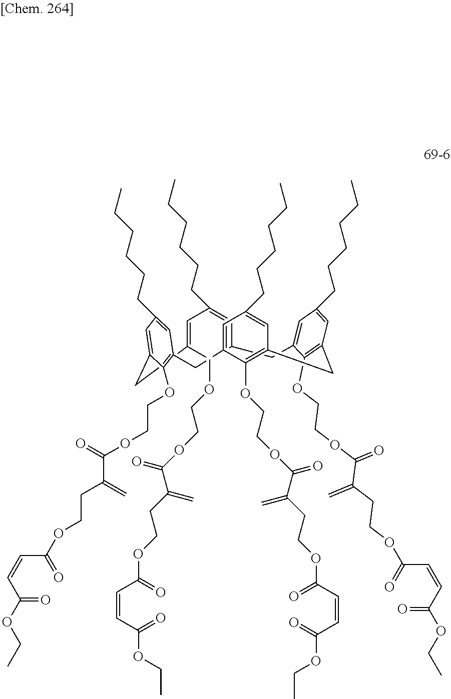 [SMILES: C=C(CCOC(=O)/C=C\C(=O)OCC)C(=O)OCCOc1c2cc(CCCCCC)cc1Cc1cc(CCCCCC)cc(c1OCCOC(=O)C(=C)CCOC(=O)/C=C\C(=O)OCC)Cc1cc(CCCCCC)cc(c1OCCOC(=O)C(=C)CCOC(=O)/C=C\C(=O)OCC)Cc1cc(CCCCCC)cc(c1OCCOC(=O)C(=C)CCOC(=O)/C=C\C(=O)OCC)C2]

### Synthesis Example 44

A 1 L four-necked flask equipped with a stirrer, a dropping funnel, a thermometer, and a reflux condenser tube was charged with sodium hydride (7.54 g, 188.4 mmol) in a nitrogen atmosphere, and mineral oil was removed by washing with hexane. Then dry DMF (160 mL) and hexyl bromide (37.2 g, 207.4 mmol) were added, and the mixture was heated to 70° C. under stirring. A solution prepared by dissolving intermediate A (10 g, 23.6 mmol) obtained in Synthesis Example 1 in dry DMF (80 mL) was added from a dropping funnel. After completion of the addition, the mixture was continuously stirred for additional 2 hours. After cooled to room temperature, the reaction mixture was poured onto ice (300 g), and concentrated hydrochloric acid was added to acidify the aqueous solution. The resulting solution was extracted with chloroform (200 mL) twice. The chloroform solution was washed with water until the pH was 5 or higher, further washed with saturated brine, and dried over anhydrous magnesium sulfate. The solvent was removed using an evaporator, and a yellow liquid was thereby obtained. Methanol was added to the mixture under stirring to precipitate solids. The solids were collected by filtration and recrystallized using isopropyl alcohol. The obtained white crystals were vacuum dried to thereby obtain a compound represented by the following formula (11.6 g, yield 65%).

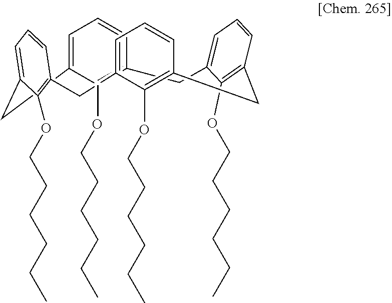 [SMILES: CCCCCCOc1c2cccc1Cc1cccc(c1OCCCCCC)Cc1cccc(c1OCCCCCC)Cc1cccc(c1OCCCCCC)C2]

### Synthesis Example 45

The same procedure as in Synthesis Example 44 was repeated except that methyl iodide was used instead of hexyl bromide and that the reaction was performed at room temperature for 24 hours, to thereby obtain a compound represented by the following formula (6.8 g, yield 60%).

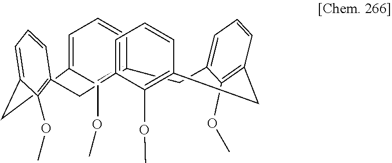 [SMILES: COc1c2cccc1Cc1cccc(c1OC)Cc1cccc(c1OC)Cc1cccc(c1OC)C2]

### Synthesis Example 46

The same procedure as in Synthesis Example 44 was repeated except that butyl bromide was used instead of hexyl bromide, to thereby obtain a compound represented by the following formula (11.0 g, yield 72%).

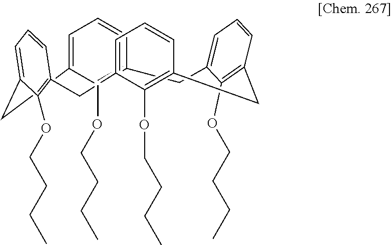 [SMILES: CCCCOc1c2cccc1Cc1cccc(c1OCCCC)Cc1cccc(c1OCCCC)Cc1cccc(c1OCCCC)C2]

### Synthesis Example 47

The same procedure as in Synthesis Example 44 was repeated except that heptyl bromide was used instead of hexyl bromide, to thereby obtain a compound represented by the following formula (14.4 g, yield 75%).

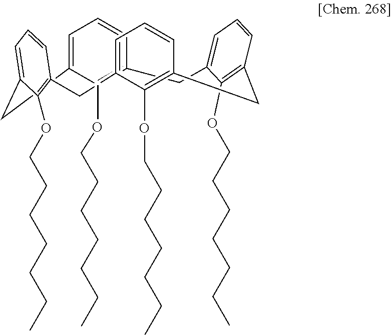 [SMILES: CCCCCCCOc1c2cccc1Cc1cccc(c1OCCCCCCC)Cc1cccc(c1OCCCCCCC)Cc1cccc(c1OCCCCCCC)C2]

### Synthesis Example 48

The same procedure as in Synthesis Example 44 was repeated except that octadecyl bromide was used instead of hexyl bromide, to thereby obtain a compound represented by the following formula (23.6 g, yield 70%).

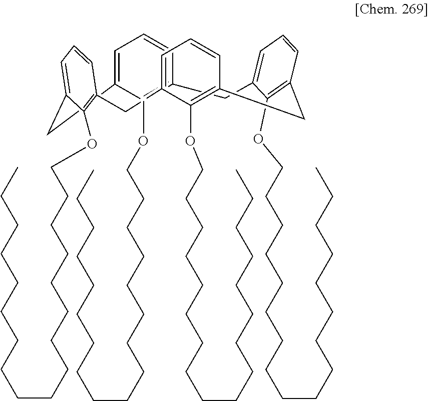 [SMILES: CCCCCCCCCCCCCCCCCCOc1c2cccc1Cc1cccc(c1OCCCCCCCCCCCCCCCCCC)Cc1cccc(c1OCCCCCCCCCCCCCCCCCC)Cc1cccc(c1OCCCCCCCCCCCCCCCCCC)C2]

### Synthesis Example 49

Referring to a known document (Organic & Biomolecular Chemistry, 13, 1708-1723; 2015), a compound represented by the following formula was synthesized in two steps using the compound obtained in Synthesis Example 44 (5.0 g, 6.57 mmol) (yield amount 3.3 g, yield 67%).

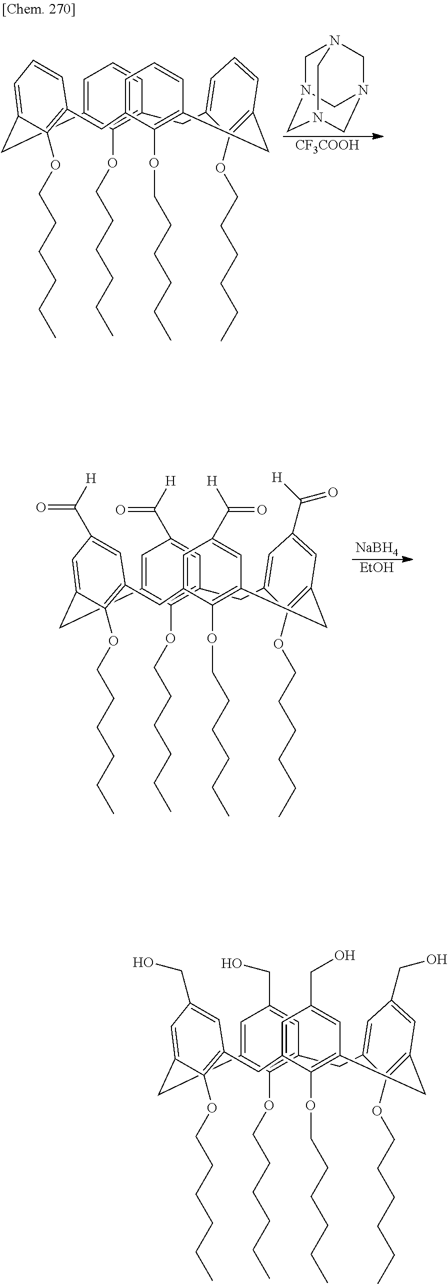

### Synthesis Example 50

The same procedure as in Synthesis Example 49 was repeated except that the compound obtained in Synthesis Example 45 (5.0 g, 10.4 mmol) was used instead of the compound obtained in Synthesis Example 44, to synthesize a compound represented by the following formula in two steps (3.75 g, yield 60%).

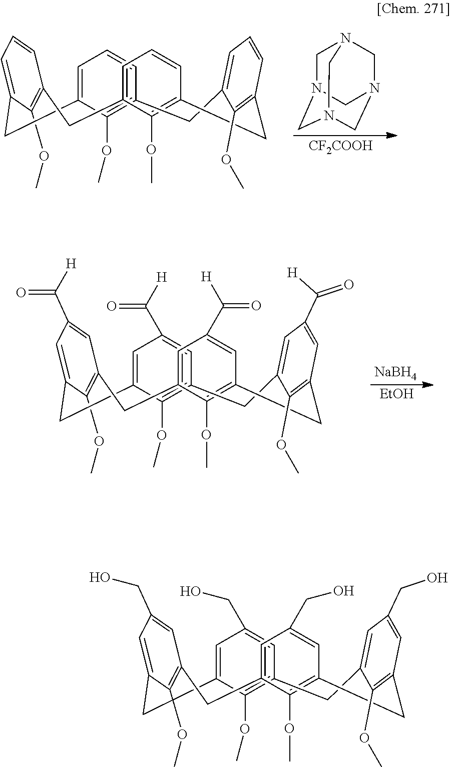

### Synthesis Example 51

The same procedure as in Synthesis Example 49 was repeated except that the compound obtained in Synthesis Example 46 (5.0 g, 7.7 mmol) was used instead of the compound obtained in Synthesis Example 44, to synthesize a compound represented by the following formula in two steps (3.73 g, yield 63%).

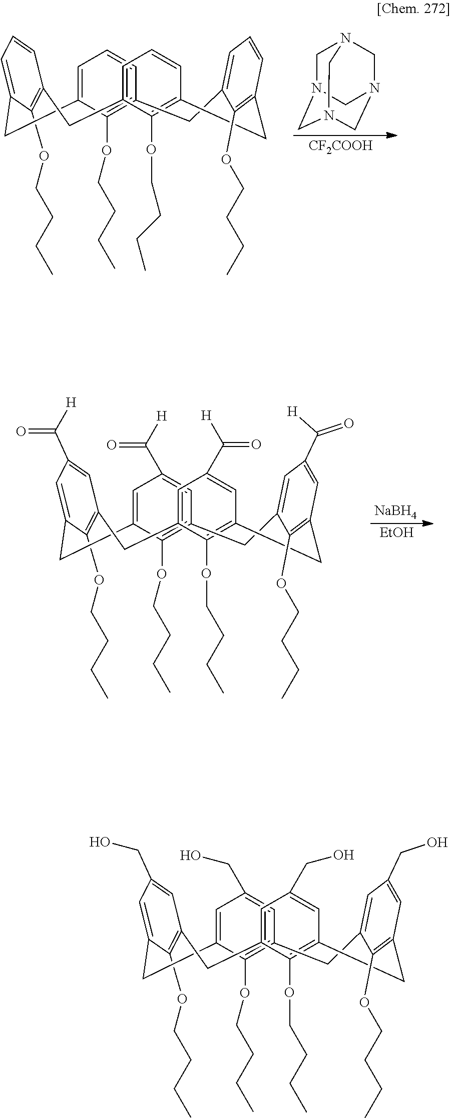

### Synthesis Example 52

The same procedure as in Synthesis Example 49 was repeated except that the compound obtained in Synthesis Example 47 (5.0 g, 6.1 mmol) was used instead of the compound obtained in Synthesis Example 44, to synthesize a compound represented by the following formula in two steps (4.01 g, yield 70%).

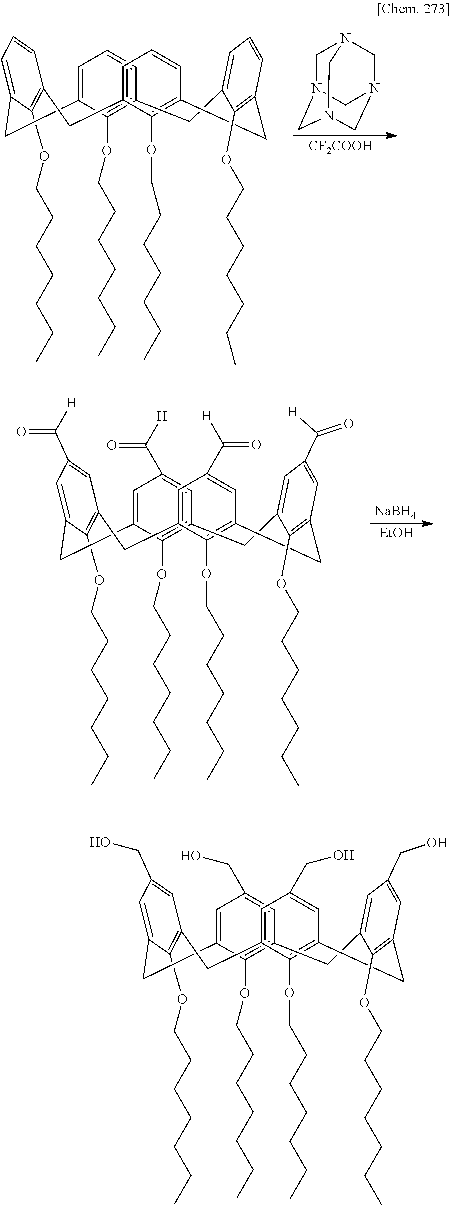

### Synthesis Example 53

The same procedure as in Synthesis Example 49 was repeated except that the compound obtained in Synthesis Example 48 (10.0 g, 7.0 mmol) was used instead of the compound obtained in Synthesis Example 44, to synthesize a compound represented by the following formula in two steps (5.96 g, yield 55%).

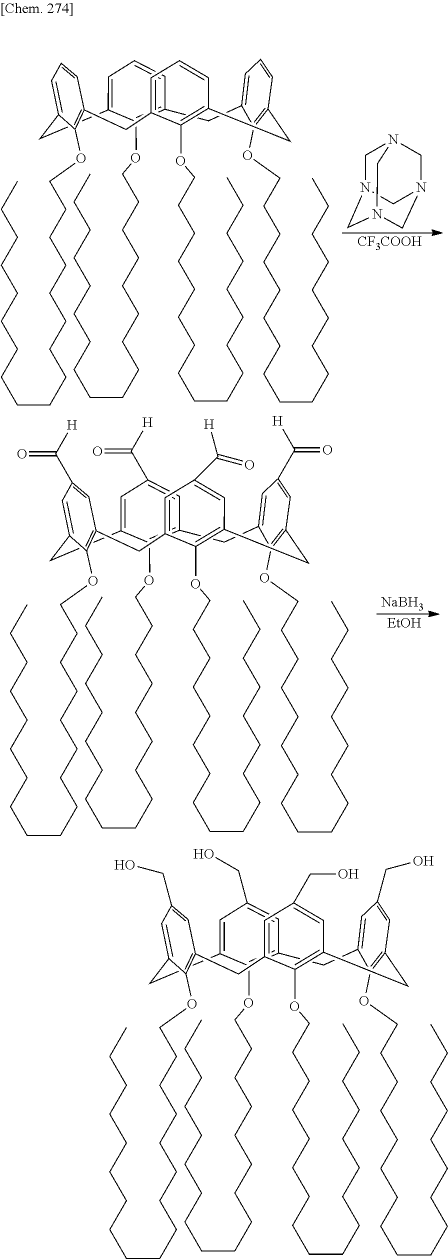

### Synthesis Example 54

A 500 mL four-necked flask equipped with a stirrer, a dropping funnel, a thermometer, and a reflux condenser tube was charged with sodium hydride (3.28 g, 82.1 mmol) in a nitrogen atmosphere, and mineral oil was removed by washing with hexane. Then dry DMF (100 mL) and hexyl bromide (16.2 g, 90.3 mmol) were added, and the mixture was heated to 70° C. under stirring. A solution prepared by dissolving 5,11,17,23-tetraallyl-25,26,27,28-tetrahydroxycalix[4]arene (6.0 g, 10.3 mmol) synthesized by a method described in a known document (The Journal of Organic Chemistry 50, 5802-58061; 1985) in dry DMF (40 mL) was added from a dropping funnel. After completion of the addition, the mixture was continuously stirred for additional 2 hours. After cooled to room temperature, the reaction mixture was poured onto ice (200 g), and concentrated hydrochloric acid was added to acidify the aqueous solution. The resulting mixture was extracted with chloroform (150 mL) twice. The chloroform solution was washed with water until the pH was 5 or higher, further washed with saturated brine, and dried over anhydrous magnesium sulfate. The solvent was removed using an evaporator, and a yellow liquid was thereby obtained. The yellow liquid was purified by silica gel column chromatography to thereby obtain a colorless transparent solution. Then a compound represented by the following formula was obtained as a white solid by recrystallization (6.6 g, yield 70%).

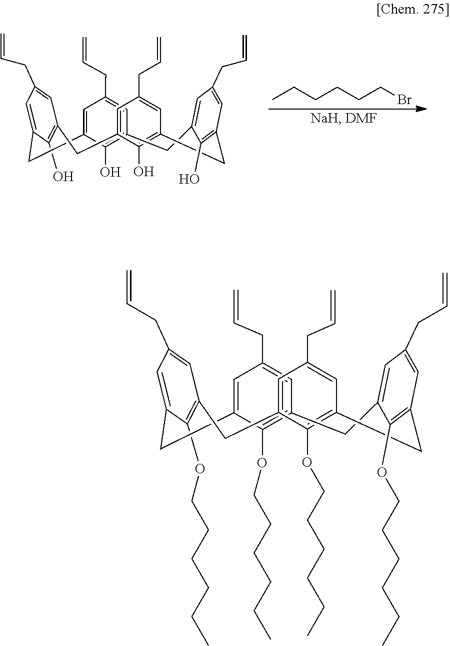

### Synthesis Example 55

The same procedure as in Synthesis Example 54 was repeated except that methyl iodide was used instead of hexyl bromide and that the reaction was performed at room temperature for 24 hours, to thereby obtain a compound represented by the following formula (4.27 g, yield 65%).

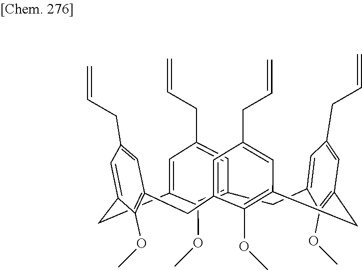 [SMILES: C=CCc1cc2c(OC)c(c1)Cc1cc(CC=C)cc(c1OC)Cc1cc(CC=C)cc(c1OC)Cc1cc(CC=C)cc(c1OC)C2]

### Synthesis Example 56

The same procedure as in Synthesis Example 54 was repeated except that butyl bromide was used instead of hexyl bromide, to thereby obtain a compound represented by the following formula (6.23 g, yield 75%).

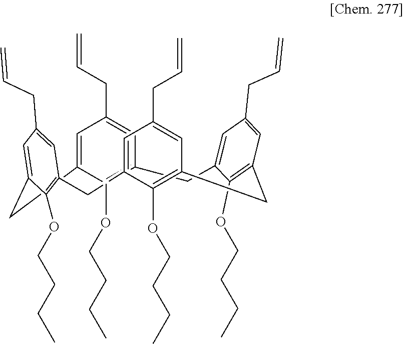 [SMILES: C=CCc1cc2c(OCCCC)c(c1)Cc1cc(CC=C)cc(c1OCCCC)Cc1cc(CC=C)cc(c1OCCCC)Cc1cc(CC=C)cc(c1OCCCC)C2]

### Synthesis Example 57

The same procedure as in Synthesis Example 54 was repeated except that heptyl bromide was used instead of hexyl bromide, to thereby obtain a compound represented by the following formula (8.02 g, yield 80%).

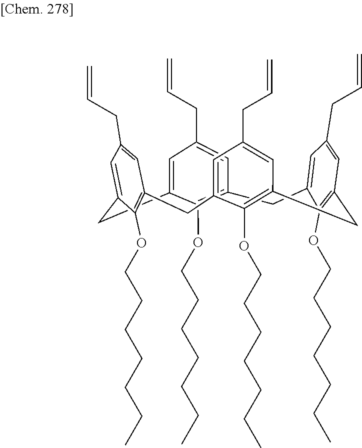 [SMILES: C=CCc1cc2c(OCCCCCCC)c(c1)Cc1cc(CC=C)cc(c1OCCCCCCC)Cc1cc(CC=C)cc(c1OCCCCCCC)Cc1cc(CC=C)cc(c1OCCCCCCC)C2]

### Synthesis Example 58

The same procedure as in Synthesis Example 54 was repeated except that octadecyl bromide was used instead of hexyl bromide, to thereby obtain a compound represented by the following formula (12.8 g, yield 75%).

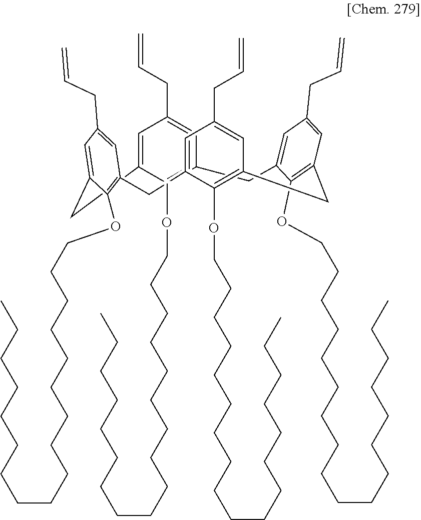 [SMILES: C=CCc1cc2c(OCCCCCCCCCCCCCCCCCC)c(c1)Cc1cc(CC=C)cc(c1OCCCCCCCCCCCCCCCCCC)Cc1cc(CC=C)cc(c1OCCCCCCCCCCCCCCCCCC)Cc1cc(CC=C)cc(c1OCCCCCCCCCCCCCCCCCC)C2]

### Synthesis Example 59

Referring to a known document (The Journal of Organic Chemistry, 67, 4722-4733; 2002), a compound represented by the following formula was synthesized using the compound obtained in Synthesis Example 54 (4 g, 4.34 mmol) (yield amount 2.93 g, yield 68%).

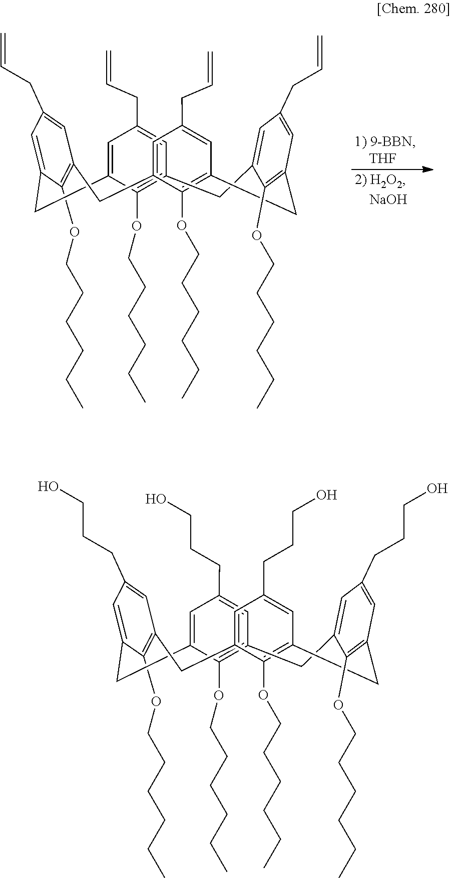 [SMILES: C=CCc1cc2c(OCCCCCC)c(c1)Cc1cc(CC=C)cc(c1OCCCCCC)Cc1cc(CC=C)cc(c1OCCCCCC)Cc1cc(CC=C)cc(c1OCCCCCC)C2 ; CCCCCCOc1c2cc(CCCO)cc1Cc1cc(CCCO)cc(c1OCCCCCC)Cc1cc(CCCO)cc(c1OCCCCCC)Cc1cc(CCCO)cc(c1OCCCCCC)C2]

### Synthesis Example 60

The same procedure as in Synthesis Example 59 was repeated except that the compound obtained in Synthesis Example 55 (4.0 g, 6.24 mmol) was used instead of the compound obtained in Synthesis Example 54, to thereby obtain a compound represented by the following formula (4.5 g, yield 72%).

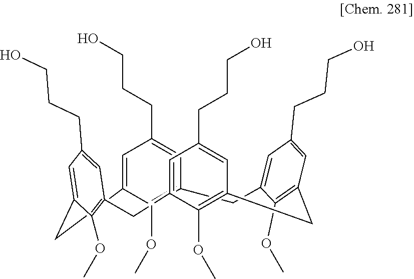 [SMILES: COc1c2cc(CCCO)cc1Cc1cc(CCCO)cc(c1OC)Cc1cc(CCCO)cc(c1OC)Cc1cc(CCCO)cc(c1OC)C2]

### Synthesis Example 61

The same procedure as in Synthesis Example 59 was repeated except that the compound obtained in Synthesis Example 56 (4.0 g, 4.94 mmol) was used instead of the compound obtained in Synthesis Example 54, to thereby obtain a compound represented by the following formula (2.59 g,

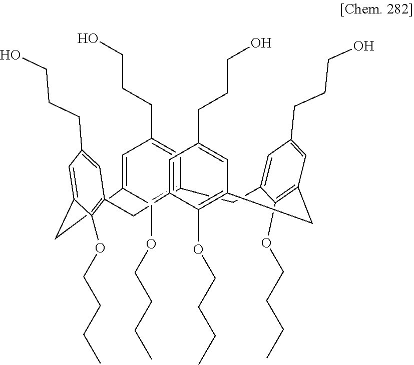 [SMILES: CCCCOc1c2cc(CCCO)cc1Cc1cc(CCCO)cc(c1OCCCC)Cc1cc(CCCO)cc(c1OCCCC)Cc1cc(CCCO)cc(c1OCCCC)C2]

### Synthesis Example 62

The same procedure as in Synthesis Example 59 was repeated except that the compound obtained in Synthesis Example 57 (4.0 g, 4.11 mmol) was used instead of the compound obtained in Synthesis Example 54, to thereby obtain a compound represented by the following formula (3.23 g, yield 75%).

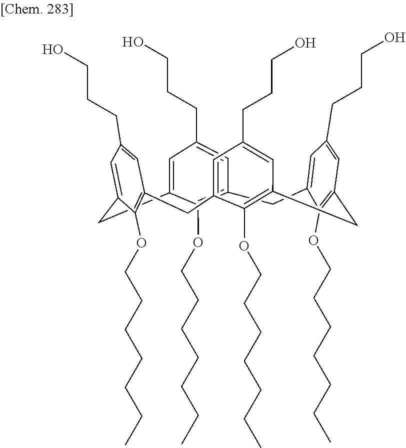 [SMILES: CCCCCCCOc1c2cc(CCCO)cc1Cc1cc(CCCO)cc(c1OCCCCCCC)Cc1cc(CCCO)cc(c1OCCCCCCC)Cc1cc(CCCO)cc(c1OCCCCCCC)C2]

### Synthesis Example 63

The same procedure as in Synthesis Example 59 was repeated except that the compound obtained in Synthesis Example 57 (8.0 g, 5.02 mmol) was used instead of the compound obtained in Synthesis Example 54, to thereby obtain a compound represented by the following formula (5.1 g, yield 61%).

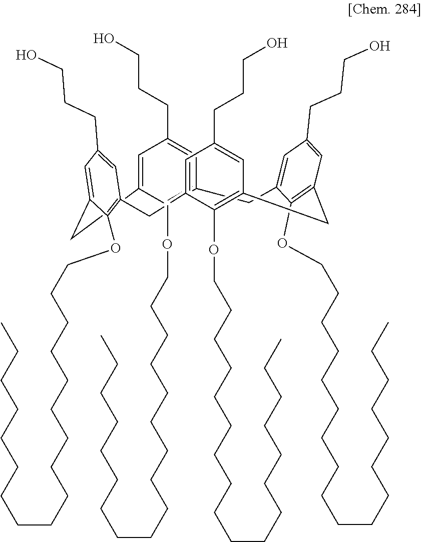 [SMILES: CCCCCCCCCCCCCCCCCCOc1c2cc(CCCO)cc1Cc1cc(CCCO)cc(c1OCCCCCCCCCCCCCCCCCC)Cc1cc(CCCO)cc(c1OCCCCCCCCCCCCCCCCCC)Cc1cc(CCCO)cc(c1OCCCCCCCCCCCCCCCCCC)C2]

### Example 35

A 100 mL four-necked flask equipped with a stirrer, a dropping funnel, and a thermometer was charged, in a nitrogen atmosphere, with the compound obtained in Synthesis Example 49 (3.0 g, 3.94 mmol), triphenylphosphine (6.201 g, 23.64 mmol), acrylic acid (0.852 g, 11.82 mmol), monomethyl maleate (1.538 g, 11.82 mmol), and 57.0 mL of tetrahydrofuran, and the mixture was stirred. Then diisopropyl azodicarboxylate (4.78 g, 23.64 mmol) was added dropwise to the mixture placed in an ice bath over 30 minutes, and the mixture was further stirred at room temperature for 24 hours. The reaction solution was concentrated in an evaporator, and hexane was added to precipitate and remove by-products such as triphenylphosphine. The obtained yellow viscous liquid was purified by silica gel column chromatography, and the target compounds 01-6, 02-6, 03-6, and 04-6 were obtained as follows. 01-6 (0.762 g, yield 15.2%). A mixture of 02-6 and 03-6 (2.501 g, yield 52.3%). 04-6 (0.615 g, yield 13.5%).

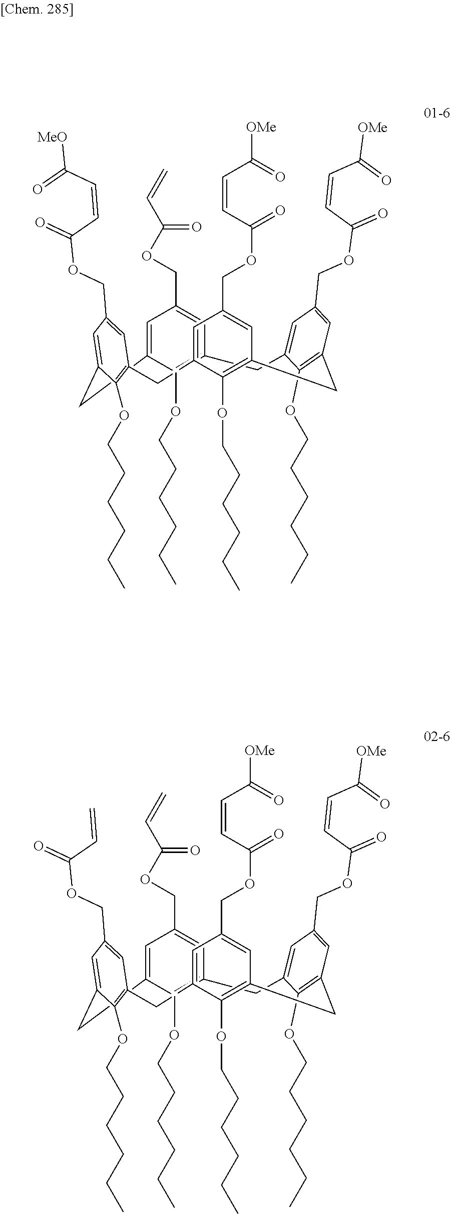 [SMILES: C=CC(=O)OCc1cc2c(OCCCCCC)c(c1)Cc1cc(COC(=O)/C=C\C(=O)OC)cc(c1OCCCCCC)Cc1cc(COC(=O)/C=C\C(=O)OC)cc(c1OCCCCCC)Cc1cc(COC(=O)/C=C\C(=O)OC)cc(c1OCCCCCC)C2 ; C=CC(=O)OCc1cc2c(OCCCCCC)c(c1)Cc1cc(COC(=O)/C=C\C(=O)OC)cc(c1OCCCCCC)Cc1cc(COC(=O)/C=C\C(=O)OC)cc(c1OCCCCCC)Cc1cc(COC(=O)C=C)cc(c1OCCCCCC)C2]

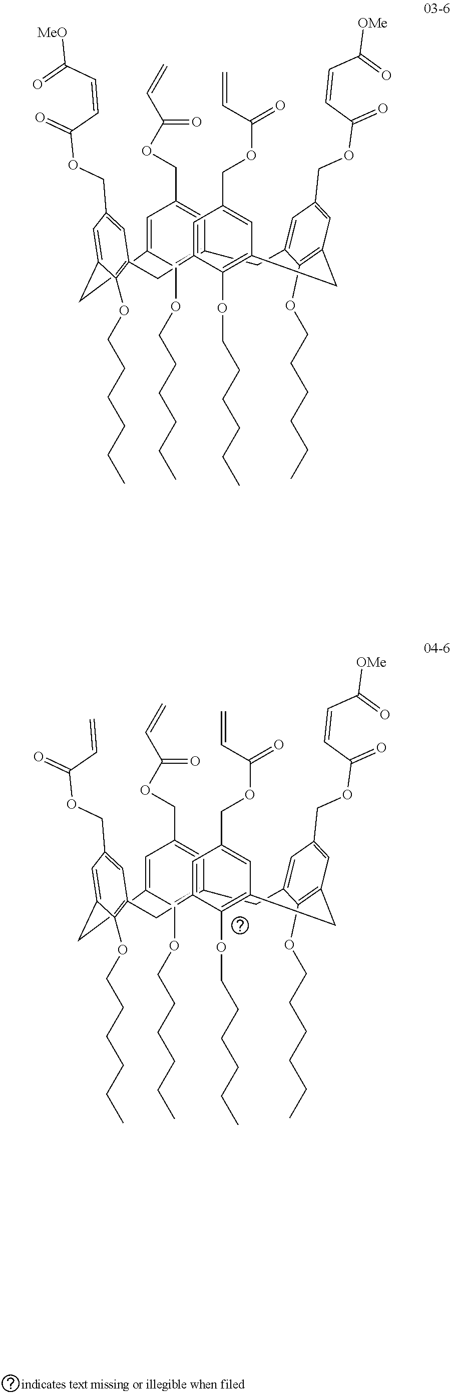 [SMILES: C=CC(=O)OCc1cc2c(OCCCCCC)c(c1)Cc1cc(COC(=O)/C=C\C(=O)OC)cc(c1OCCCCCC)Cc1cc(COC(=O)C=C)cc(c1OCCCCCC)Cc1cc(COC(=O)/C=C\C(=O)OC)cc(c1OCCCCCC)C2 ; C=CC(=O)OCc1cc2c(OCCCCCC)c(c1)Cc1cc(COC(=O)C=C)cc(c1OCCCCCC)Cc1cc(COC(=O)/C=C\C(=O)OC)cc(c1OCCCCCC)Cc1cc(COC(=O)C=C)cc(c1OCCCCCC)C2]

### Example 36

The same procedure as in Example 35 was repeated except that the compound obtained in Synthesis Example 50 (3.0 g, 4.99 mmol) was used instead of the compound obtained in Synthesis Example 49, to thereby obtain the target compounds 01-1, 02-1, 03-1, and 04-1 as follows. 01-1 (0.723 g, yield 14.6%). A mixture of 02-1 and 03-1 (2.40 g, yield 51.5%). 04-1 (0.721 g, yield 16.5%).

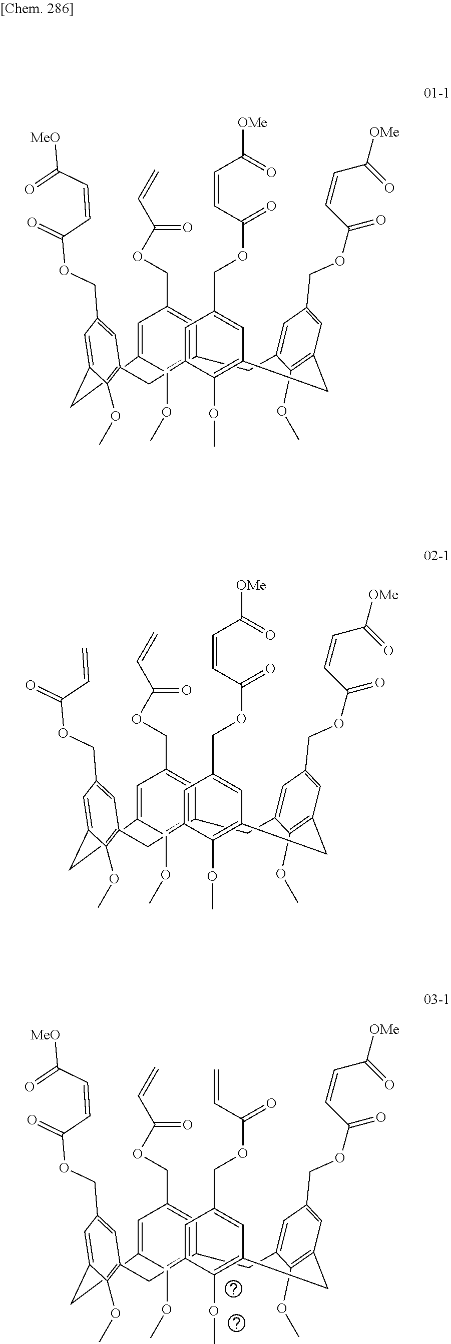 [SMILES: C=CC(=O)OCc1cc2c(OC)c(c1)Cc1cc(COC(=O)/C=C\C(=O)OC)cc(c1OC)Cc1cc(COC(=O)/C=C\C(=O)OC)cc(c1OC)Cc1cc(COC(=O)/C=C\C(=O)OC)cc(c1OC)C2 ; C=CC(=O)OCc1cc2c(OC)c(c1)Cc1cc(COC(=O)/C=C\C(=O)OC)cc(c1OC)Cc1cc(COC(=O)/C=C\C(=O)OC)cc(c1OC)Cc1cc(COC(=O)C=C)cc(c1OC)C2 ; C=CC(=O)OCc1cc2c(OC)c(c1)Cc1cc(COC(=O)/C=C\C(=O)OC)cc(c1OC)Cc1cc(COC(=O)C=C)cc(c1OC)Cc1cc(COC(=O)/C=C\C(=O)OC)cc(c1OC)C2]

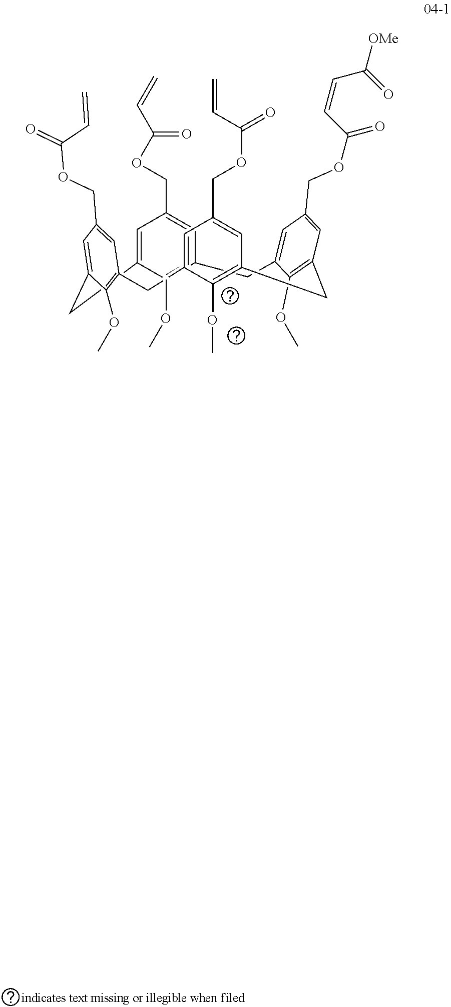 [SMILES: C=CC(=O)OCc1cc2c(OC)c(c1)Cc1cc(COC(=O)C=C)cc(c1OC)Cc1cc(COC(=O)/C=C\C(=O)OC)cc(c1OC)Cc1cc(COC(=O)C=C)cc(c1OC)C2]

### Example 37

The same procedure as in Example 35 was repeated except that the compound obtained in Synthesis Example 51 (3.0 g, 3.9 mmol) was used instead of the compound obtained in Synthesis Example 49, to thereby obtain the target compounds 01-4, 02-4, 03-4, and 04-4 as follows. 01-4 (0.705 g, yield 15.6%). A mixture of 02-4 and 03-4 (2.303 g, yield 53.6%). 04-4 (0.602 g, yield 14.8%).

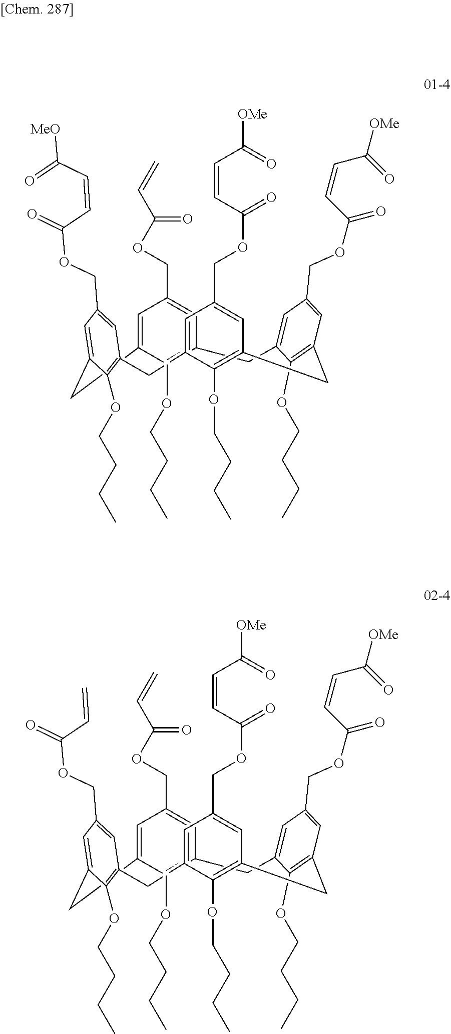 [SMILES: C=CC(=O)OCc1cc2c(OCCCC)c(c1)Cc1cc(COC(=O)/C=C\C(=O)OC)cc(c1OCCCC)Cc1cc(COC(=O)/C=C\C(=O)OC)cc(c1OCCCC)Cc1cc(COC(=O)/C=C\C(=O)OC)cc(c1OCCCC)C2 ; C=CC(=O)OCc1cc2c(OCCCC)c(c1)Cc1cc(COC(=O)/C=C\C(=O)OC)cc(c1OCCCC)Cc1cc(COC(=O)/C=C\C(=O)OC)cc(c1OCCCC)Cc1cc(COC(=O)C=C)cc(c1OCCCC)C2]

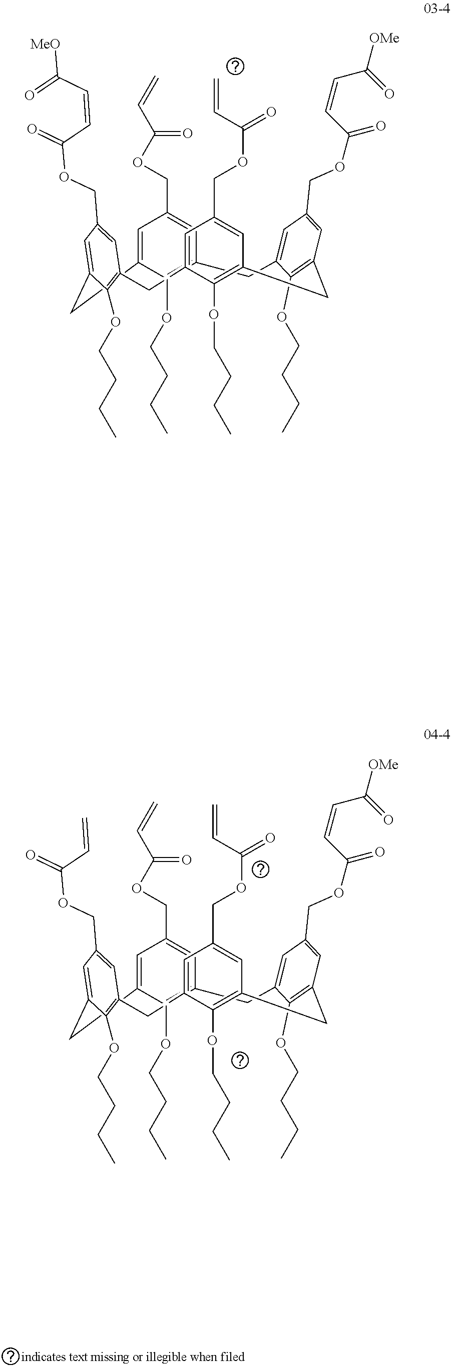 [SMILES: C=CC(=O)OCc1cc2c(OCCCC)c(c1)Cc1cc(COC(=O)/C=C\C(=O)OC)cc(c1OCCCC)Cc1cc(COC(=O)C=C)cc(c1OCCCC)Cc1cc(COC(=O)/C=C\C(=O)OC)cc(c1OCCCC)C2 ; C=CC(=O)OCc1cc2c(OCCCC)c(c1)Cc1cc(COC(=O)C=C)cc(c1OCCCC)Cc1cc(COC(=O)/C=C\C(=O)OC)cc(c1OCCCC)Cc1cc(COC(=O)C=C)cc(c1OCCCC)C2]

### Example 38

The same procedure as in Example 35 was repeated except that the compound obtained in Synthesis Example 52 (3.0 g, 3.2 mmol) was used instead of the compound obtained in Synthesis Example 49, to thereby obtain the target compounds 01-7, 02-7, 03-7, and 04-7 as follows. 01-7 (0.531 g, yield 12.5%). A mixture of 02-7 and 03-7 (2.296 g, yield 56.5%). 04-7 (0.535 g, yield 13.8%).

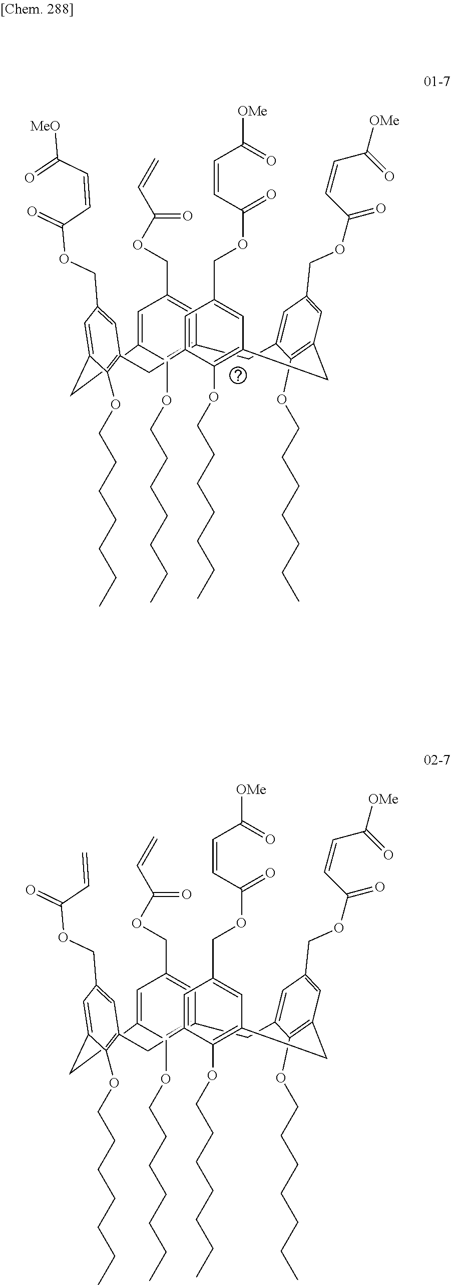 [SMILES: C=CC(=O)OCc1cc2c(OCCCCCCC)c(c1)Cc1cc(COC(=O)/C=C\C(=O)OC)cc(c1OCCCCCCC)Cc1cc(COC(=O)/C=C\C(=O)OC)cc(c1OCCCCCCC)Cc1cc(COC(=O)/C=C\C(=O)OC)cc(c1OCCCCCCC)C2 ; C=CC(=O)OCc1cc2c(OCCCCCCC)c(c1)Cc1cc(COC(=O)/C=C\C(=O)OC)cc(c1OCCCCCCC)Cc1cc(COC(=O)/C=C\C(=O)OC)cc(c1OCCCCCCC)Cc1cc(COC(=O)C=C)cc(c1OCCCCCCC)C2]

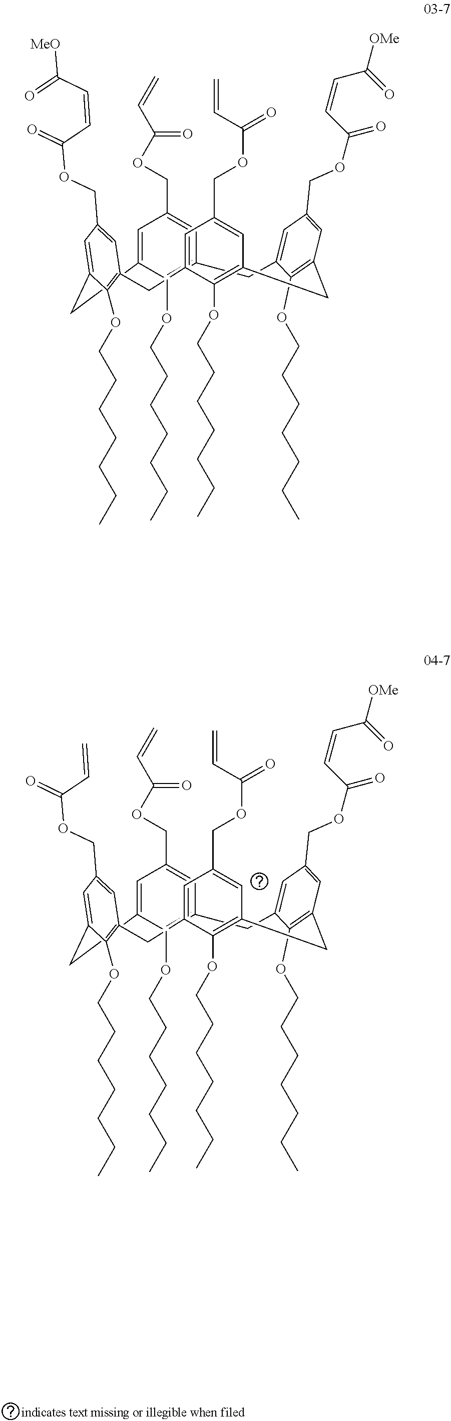 [SMILES: C=CC(=O)OCc1cc2c(OCCCCCCC)c(c1)Cc1cc(COC(=O)/C=C\C(=O)OC)cc(c1OCCCCCCC)Cc1cc(COC(=O)C=C)cc(c1OCCCCCCC)Cc1cc(COC(=O)/C=C\C(=O)OC)cc(c1OCCCCCCC)C2 ; C=CC(=O)OCc1cc2c(OCCCCCCC)c(c1)Cc1cc(COC(=O)C=C)cc(c1OCCCCCCC)Cc1cc(COC(=O)/C=C\C(=O)OC)cc(c1OCCCCCCC)Cc1cc(COC(=O)C=C)cc(c1OCCCCCCC)C2]

### Example 39

The same procedure as in Example 35 was repeated except that the compound obtained in Synthesis Example 53 (3.0 g, 1.93 mmol) was used instead of the compound obtained in Synthesis Example 49, to thereby obtain the target compounds 01-18, 02-18, 03-18, and 04-18 as follows. 01-18 (0.42 g, yield 11.2%). A mixture of 02-18 and 03-18 (1.832 g, yield 50.3%). 04-18 (0.476 g, yield 13.5%).

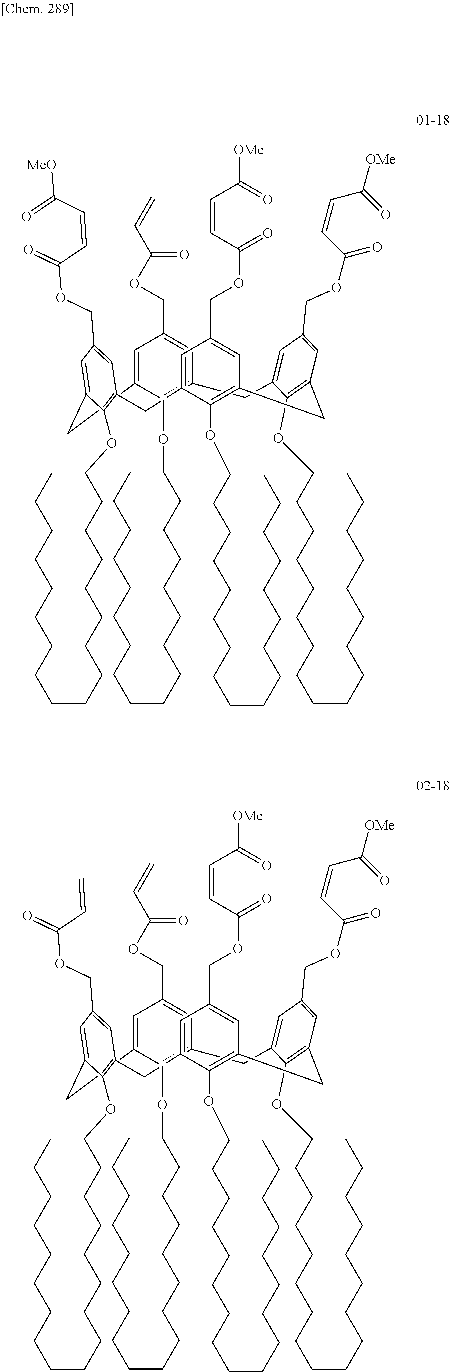 [SMILES: C=CC(=O)OCc1cc2c(OCCCCCCCCCCCCCCCCCC)c(c1)Cc1cc(COC(=O)/C=C\C(=O)OC)cc(c1OCCCCCCCCCCCCCCCCCC)Cc1cc(COC(=O)/C=C\C(=O)OC)cc(c1OCCCCCCCCCCCCCCCCCC)Cc1cc(COC(=O)/C=C\C(=O)OC)cc(c1OCCCCCCCCCCCCCCCCCC)C2 ; C=CC(=O)OCc1cc2c(OCCCCCCCCCCCCCCCCCC)c(c1)Cc1cc(COC(=O)/C=C\C(=O)OC)cc(c1OCCCCCCCCCCCCCCCCCC)Cc1cc(COC(=O)/C=C\C(=O)OC)cc(c1OCCCCCCCCCCCCCCCCCC)Cc1cc(COC(=O)C=C)cc(c1OCCCCCCCCCCCCCCCCCC)C2]

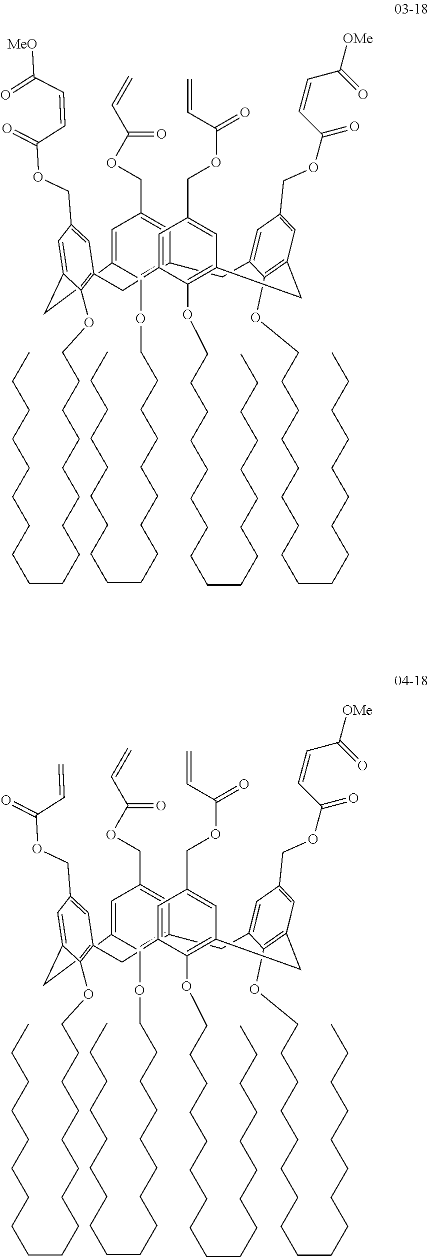 [SMILES: C=CC(=O)OCc1cc2c(OCCCCCCCCCCCCCCCCCC)c(c1)Cc1cc(COC(=O)/C=C\C(=O)OC)cc(c1OCCCCCCCCCCCCCCCCCC)Cc1cc(COC(=O)C=C)cc(c1OCCCCCCCCCCCCCCCCCC)Cc1cc(COC(=O)/C=C\C(=O)OC)cc(c1OCCCCCCCCCCCCCCCCCC)C2 ; C=CC(=O)OCc1cc2c(OCCCCCCCCCCCCCCCCCC)c(c1)Cc1cc(COC(=O)C=C)cc(c1OCCCCCCCCCCCCCCCCCC)Cc1cc(COC(=O)/C=C\C(=O)OC)cc(c1OCCCCCCCCCCCCCCCCCC)Cc1cc(COC(=O)C=C)cc(c1OCCCCCCCCCCCCCCCCCC)C2]

### Synthesis Example 64

A 100 mL four-necked flask equipped with a stirrer, a thermometer, and a reflux condenser tube was charged with 2.00 g (2.27 mmol) of the compound obtained in Synthesis Example 49, 3.57 g (13.62 mmol) of triphenylphosphine, 2.95 g (13.62 mmol) of 2-[[[(1,1-dimethylethyl)dimethylsilyl]oxy]2-propenoic acid, and 38 mL of tetrahydrofuran, and the mixture was stirred. Then 2.75 g (13.62 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 2.85 g, yield 75.0%).

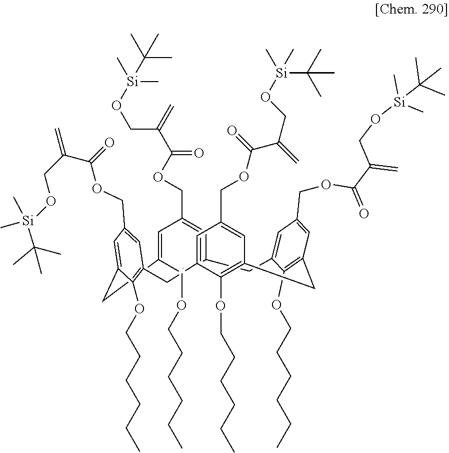 [SMILES: C=C(CO[Si](C)(C)C(C)(C)C)C(=O)OCc1cc2c(OCCCCCC)c(c1)Cc1cc(COC(=O)C(=C)CO[Si](C)(C)C(C)(C)C)cc(c1OCCCCCC)Cc1cc(COC(=O)C(=C)CO[Si](C)(C)C(C)(C)C)cc(c1OCCCCCC)Cc1cc(COC(=O)C(=C)CO[Si](C)(C)C(C)(C)C)cc(c1OCCCCCC)C2]

### Synthesis Example 65

The same procedure as in Synthesis Example 64 was repeated except that the compound obtained in Synthesis Example 50 (2.00 g, 3.33 mmol) was used instead of the compound obtained in Synthesis Example 49, to thereby obtain a compound represented by the following formula (3.26 g, yield 70.2%).

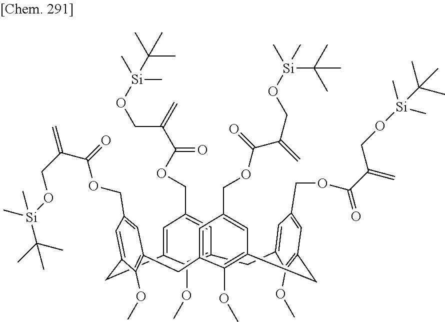 [SMILES: C=C(CO[Si](C)(C)C(C)(C)C)C(=O)OCc1cc2c(OC)c(c1)Cc1cc(COC(=O)C(=C)CO[Si](C)(C)C(C)(C)C)cc(c1OC)Cc1cc(COC(=O)C(=C)CO[Si](C)(C)C(C)(C)C)cc(c1OC)Cc1cc(COC(=O)C(=C)CO[Si](C)(C)C(C)(C)C)cc(c1OC)C2]

### Synthesis Example 66

The same procedure as in Synthesis Example 64 was repeated except that the compound obtained in Synthesis Example 51 (2.00 g, 2.60 mmol) was used instead of the compound obtained in Synthesis Example 49, to thereby obtain a compound represented by the following formula (3.12 g, yield 76.8%).

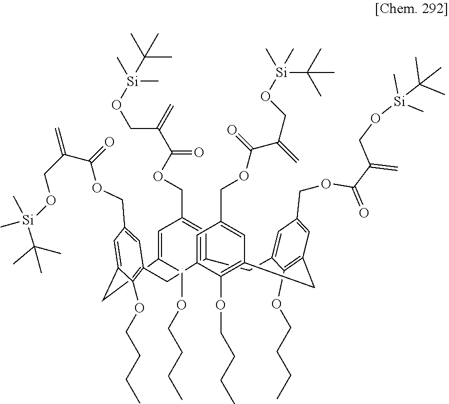 [SMILES: C=C(CO[Si](C)(C)C(C)(C)C)C(=O)OCc1cc2c(OCCCC)c(c1)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)C2]

### Synthesis Example 67

The same procedure as in Synthesis Example 64 was repeated except that the compound obtained in Synthesis Example 52 (2.00 g, 2.13 mmol) was used instead of the compound obtained in Synthesis Example 49, to thereby obtain a compound represented by the following formula (2.74 g, yield 74.2%).

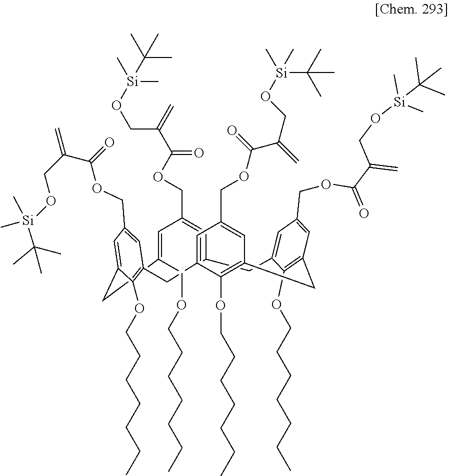 [SMILES: C=C(CO[Si](C)(C)C(C)(C)C)C(=O)OCc1cc2c(OCCCCCCC)c(c1)Cc1cc(COC(=O)C(=C)CO[Si](C)(C)C(C)(C)C)cc(c1OCCCCCCC)Cc1cc(COC(=O)C(=C)CO[Si](C)(C)C(C)(C)C)cc(c1OCCCCCCC)Cc1cc(COC(=O)C(=C)CO[Si](C)(C)C(C)(C)C)cc(c1OCCCCCCC)C2]

### Synthesis Example 68

The same procedure as in Synthesis Example 62 was repeated except that the compound obtained in Synthesis Example 53 (2.00 g, 1.29 mmol) was used instead of the compound obtained in Synthesis Example 49, to thereby obtain a compound represented by the following formula (2.58 g, yield 85.3%).

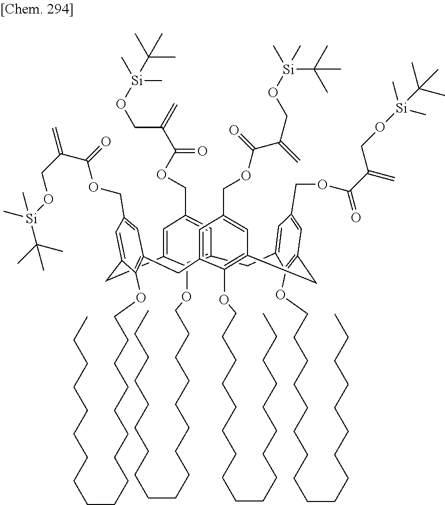 [SMILES: C=C(CO[Si](C)(C)C(C)(C)C)C(=O)OCc1cc2c(OCCCCCCCCCCCCCCCCCC)c(c1)Cc1cc(COC(=O)C(=C)CO[Si](C)(C)C(C)(C)C)cc(c1OCCCCCCCCCCCCCCCCCC)Cc1cc(COC(=O)C(=C)CO[Si](C)(C)C(C)(C)C)cc(c1OCCCCCCCCCCCCCCCCCC)Cc1cc(COC(=O)C(=C)CO[Si](C)(C)C(C)(C)C)cc(c1OCCCCCCCCCCCCCCCCCC)C2]

### Synthesis Example 69

A 100 mL four-necked flask equipped with a stirrer, a thermometer, and a reflux condenser tube was charged with 2.50 g (1.49 mmol) of the compound obtained in Synthesis Example 64, 0.538 g (8.96 mmol) of acetic acid, and 60 mL of tetrahydrofuran, and the mixture was stirred. A colorless transparent solution. Next, tetrabutylammonium fluoride (an about 1 mol/L tetrahydrofuran solution 8.96 mL (8.96 mmol) was slowly added dropwise to the mixture placed in an ice bath under stirring, and the resulting mixture was further stirred at room temperature for 12 hours. A saturated aqueous ammonium chloride solution was added to the reaction mixture, and then 30 mL of chloroform was added. The reaction mixture was transferred to a separatory funnel, and the organic layer was separated. Then the aqueous layer was extracted with 30 mL of chloroform twice. The combined organic layer was washed with saturated brine and dried over anhydrous magnesium sulfate. The solvent was removed by evaporation using an evaporator, and a yellow transparent liquid was thereby obtained. The yellow transparent liquid was purified by silica gel column column chromatography to thereby obtain a compound represented by the following formula as a white solid (yield amount 1.663 g, yield 91.5%).

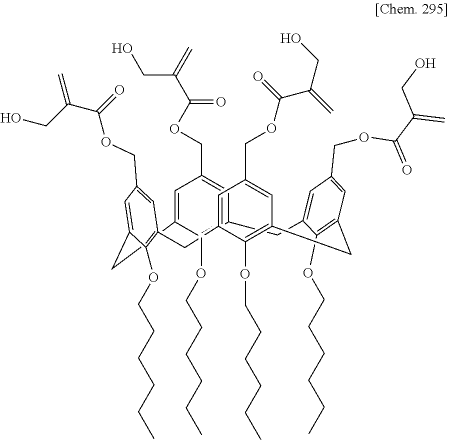 [SMILES: C=C(CO)C(=O)OCc1cc2c(OCCCCCC)c(c1)Cc1cc(COC(=O)C(=C)CO)cc(c1OCCCCCC)Cc1cc(COC(=O)C(=C)CO)cc(c1OCCCCCC)Cc1cc(COC(=O)C(=C)CO)cc(c1OCCCCCC)C2]

### Synthesis Example 70

The same procedure as in Synthesis Example 69 was repeated except that the compound obtained in Synthesis Example 65 (2.5 g, 1.79 mmol) was used instead of the compound obtained in Synthesis Example 64, to thereby obtain a compound represented by the following formula (1.551 g, yield 92.3%).

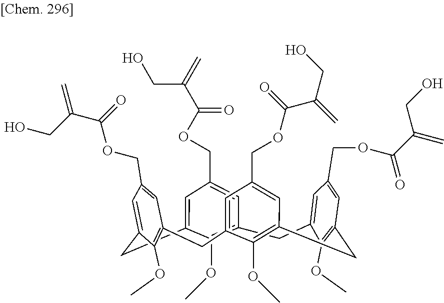 [SMILES: C=C(CO)C(=O)OCc1cc2c(OC)c(c1)Cc1cc(COC(=O)C(=C)CO)cc(c1OC)Cc1cc(COC(=O)C(=C)CO)cc(c1OC)Cc1cc(COC(=O)C(=C)CO)cc(c1OC)C2]

### Synthesis Example 71

The same procedure as in Synthesis Example 69 was repeated except that the compound obtained in Synthesis Example 66 (2.5 g, 1.60 mmol) was used instead of the compound obtained in Synthesis Example 64, to thereby obtain a compound represented by the following formula (1.671 g, yield 94.5%).

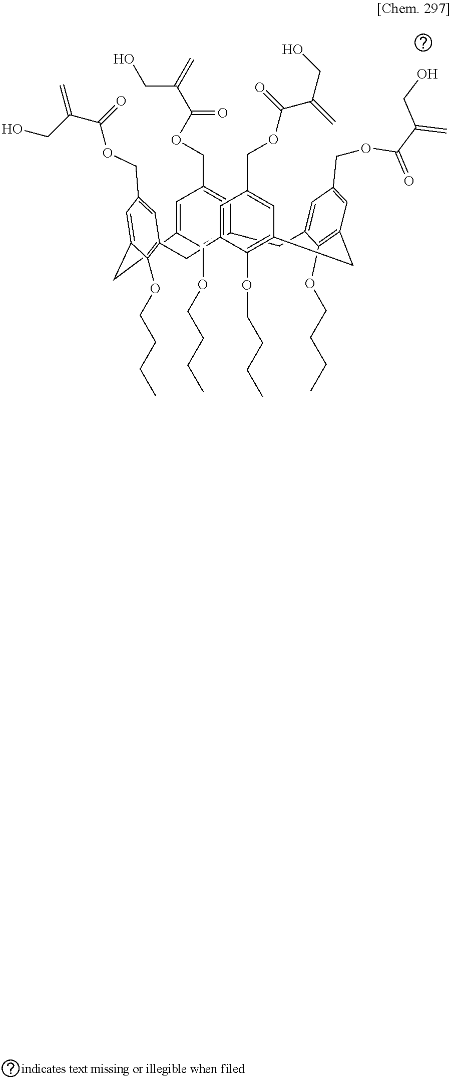 [SMILES: C=C(CO)C(=O)OCc1cc2c(OCCCC)c(c1)Cc1cc(COC(=O)C(=C)CO)cc(c1OCCCC)Cc1cc(COC(=O)C(=C)CO)cc(c1OCCCC)Cc1cc(COC(=O)C(=C)CO)cc(c1OCCCC)C2]

### Synthesis Example 72

The same procedure as in Synthesis Example 69 was repeated except that the compound obtained in Synthesis Example 67 (2.5 g, 1.44 mmol) was used instead of the compound obtained in Synthesis Example 64, to thereby obtain a compound represented by the following formula (1.759 g, yield 95.6%).

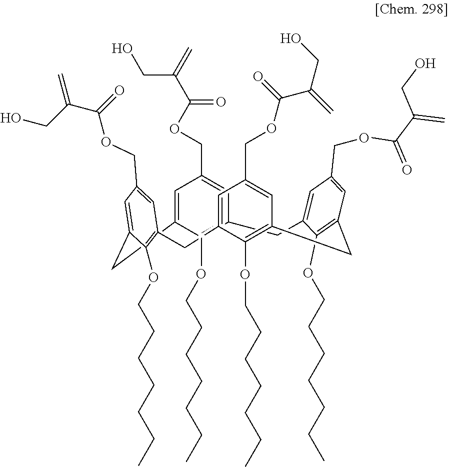 [SMILES: C=C(CO)C(=O)OCc1cc2c(OCCCCCCC)c(c1)Cc1cc(COC(=O)C(=C)CO)cc(c1OCCCCCCC)Cc1cc(COC(=O)C(=C)CO)cc(c1OCCCCCCC)Cc1cc(COC(=O)C(=C)CO)cc(c1OCCCCCCC)C2]

### Synthesis Example 73

The same procedure as in Synthesis Example 69 was repeated except that the compound obtained in Synthesis Example 68 (2.50 g, 1.06 mmol) was used instead of the compound obtained in Synthesis Example 64, to thereby obtain a compound represented by the following formula (1.90 g, yield 94.8%).

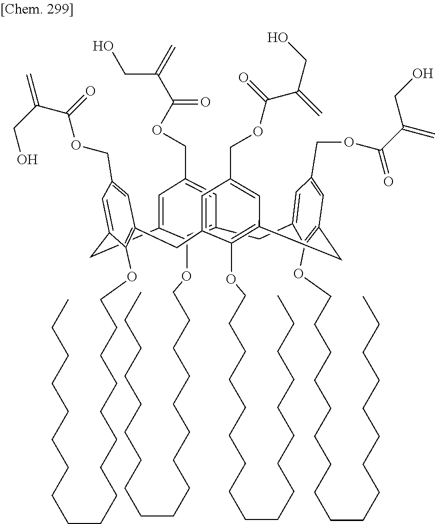 [SMILES: C=C(CO)C(=O)OCc1cc2c(OCCCCCCCCCCCCCCCCCC)c(c1)Cc1cc(COC(=O)C(=C)CO)cc(c1OCCCCCCCCCCCCCCCCCC)Cc1cc(COC(=O)C(=C)CO)cc(c1OCCCCCCCCCCCCCCCCCC)Cc1cc(COC(=O)C(=C)CO)cc(c1OCCCCCCCCCCCCCCCCCC)C2]

### Example 40

A 100 mL four-necked flask equipped with a stirrer, a thermometer, and a reflux condenser tube was charged with 1.50 g (1.23 mmol) of the compound obtained in Synthesis Example 69, triphenylphosphine (1.939 g, 7.39 mmol), monomethyl maleate (0.9617 g, 7.39 mmol), and 20 mL of tetrahydrofuran, and the mixture was stirred. Then 1.495 g (7.39 mmol) of diisopropyl azodicarboxylate was added dropwise to the mixture placed in an ice bath over 30 minutes, and the mixture was further stirred at room temperature for 24 hours. The reaction solution was concentrated in an evaporator, and hexane was added to precipitate and remove by-products such as triphenylphosphine. The obtained yellow viscous liquid was purified by silica gel column chromatography to thereby obtain the target compound 05-6 (yield amount 1.757 g, yield 85.6%).

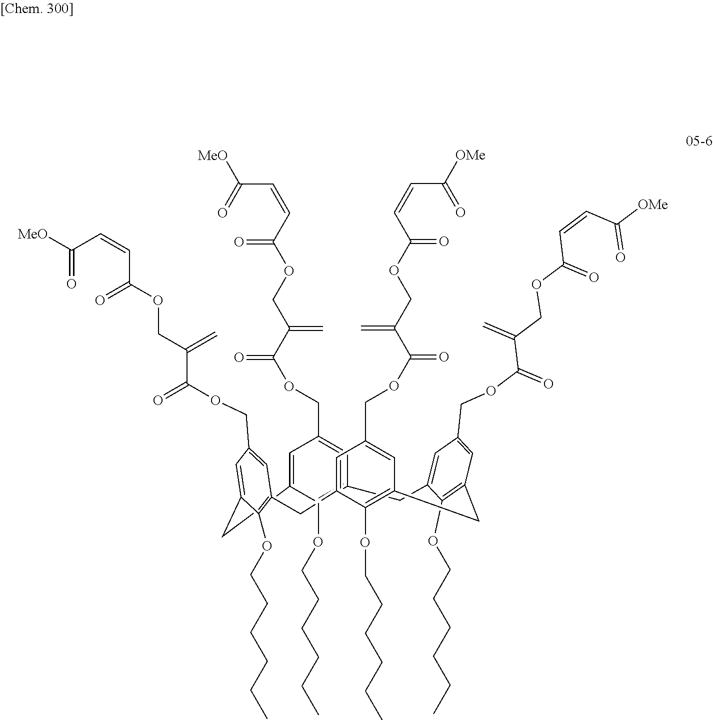 [SMILES: C=C(COC(=O)/C=C\C(=O)OC)C(=O)OCc1cc2c(OCCCCCC)c(c1)Cc1cc(COC(=O)C(=C)COC(=O)/C=C\C(=O)OC)cc(c1OCCCCCC)Cc1cc(COC(=O)C(=C)COC(=O)/C=C\C(=O)OC)cc(c1OCCCCCC)Cc1cc(COC(=O)C(=C)COC(=O)/C=C\C(=O)OC)cc(c1OCCCCCC)C2]

### Example 41

The same procedure as in Example 40 was repeated except that the compound obtained in Synthesis Example 70 (1.50 g, 1.60 mmol) was used instead of the compound obtained in Synthesis Example 69, to thereby obtain the target compound 05-1 (1.85 g, yield 83.4%).

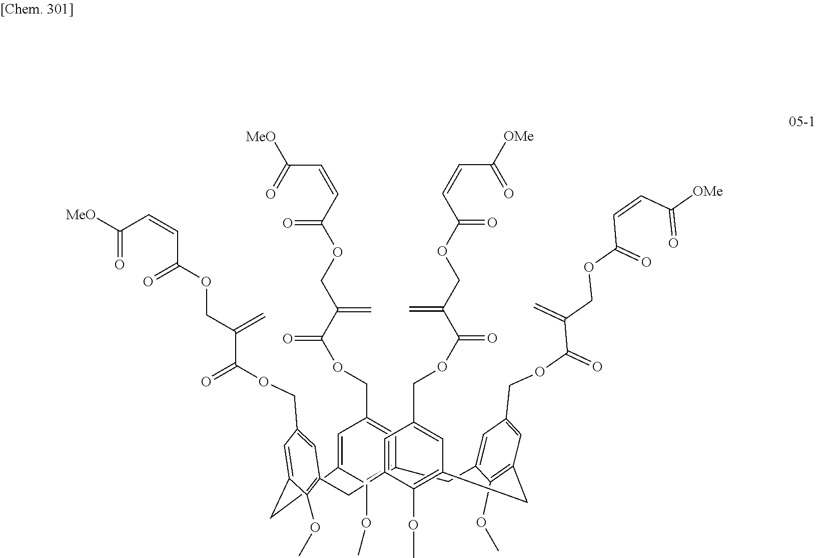 [SMILES: C=C(COC(=O)/C=C\C(=O)OC)C(=O)OCc1cc2c(OC)c(c1)Cc1cc(COC(=O)C(=C)COC(=O)/C=C\C(=O)OC)cc(c1OC)Cc1cc(COC(=O)C(=C)COC(=O)/C=C\C(=O)OC)cc(c1OC)Cc1cc(COC(=O)C(=C)COC(=O)/C=C\C(=O)OC)cc(c1OC)C2]

### Example 42

The same procedure as in Example 40 was repeated except that the compound obtained in Synthesis Example 71 (1.50 g, 1.36 mmol) was used instead of the compound obtained in Synthesis Example 69, to thereby obtain the target compound 05-4 (0.861 g, yield 55.6%).

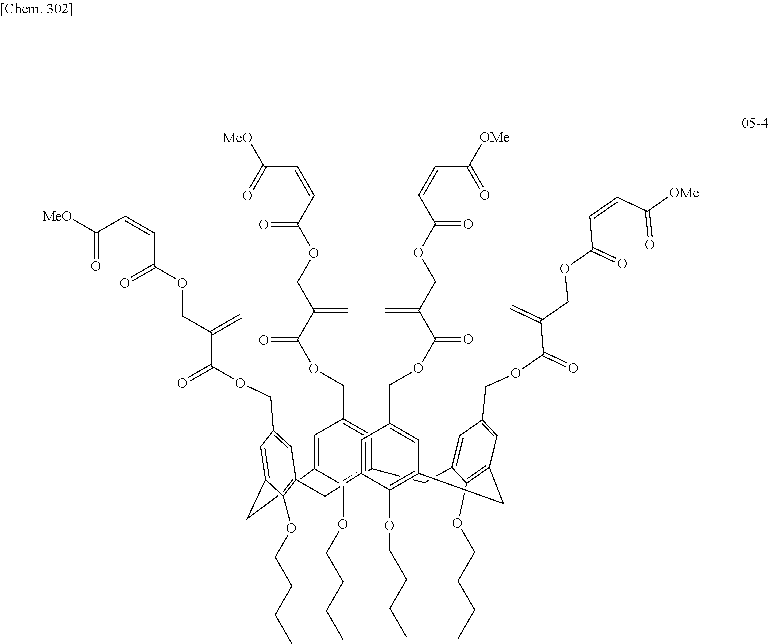 [SMILES: C=C(COC(=O)/C=C\C(=O)OC)C(=O)OCc1cc2c(OCCCC)c(c1)Cc1cc(COC(=O)C(=C)COC(=O)/C=C\C(=O)OC)cc(c1OCCCC)Cc1cc(COC(=O)C(=C)COC(=O)/C=C\C(=O)OC)cc(c1OCCCC)Cc1cc(COC(=O)C(=C)COC(=O)/C=C\C(=O)OC)cc(c1OCCCC)C2]

### Example 43

The same procedure as in Example 40 was repeated except that the compound obtained in Synthesis Example 72 (1.50 g, 1.18 mmol) was used instead of the compound obtained in Synthesis Example 69, to thereby obtain the target compound 05-7 (1.835 g, yield 90.5%).

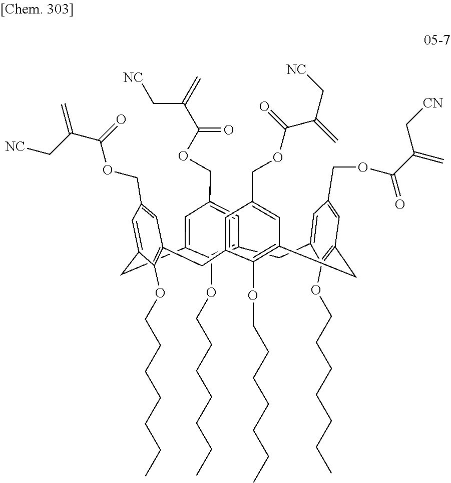 [SMILES: C=C(CC#N)C(=O)OCc1cc2c(OCCCCCCC)c(c1)Cc1cc(COC(=O)C(=C)CC#N)cc(c1OCCCCCCC)Cc1cc(COC(=O)C(=C)CC#N)cc(c1OCCCCCCC)Cc1cc(COC(=O)C(=C)CC#N)cc(c1OCCCCCCC)C2]

### Example 44

The same procedure as in Example 40 was repeated except that the compound obtained in Synthesis Example 73 (1.5 g, 0.79 mmol) was used instead of the compound obtained in Synthesis Example 69, to thereby obtain the target compound 05-18 (1.455 g, yield 78.4%).

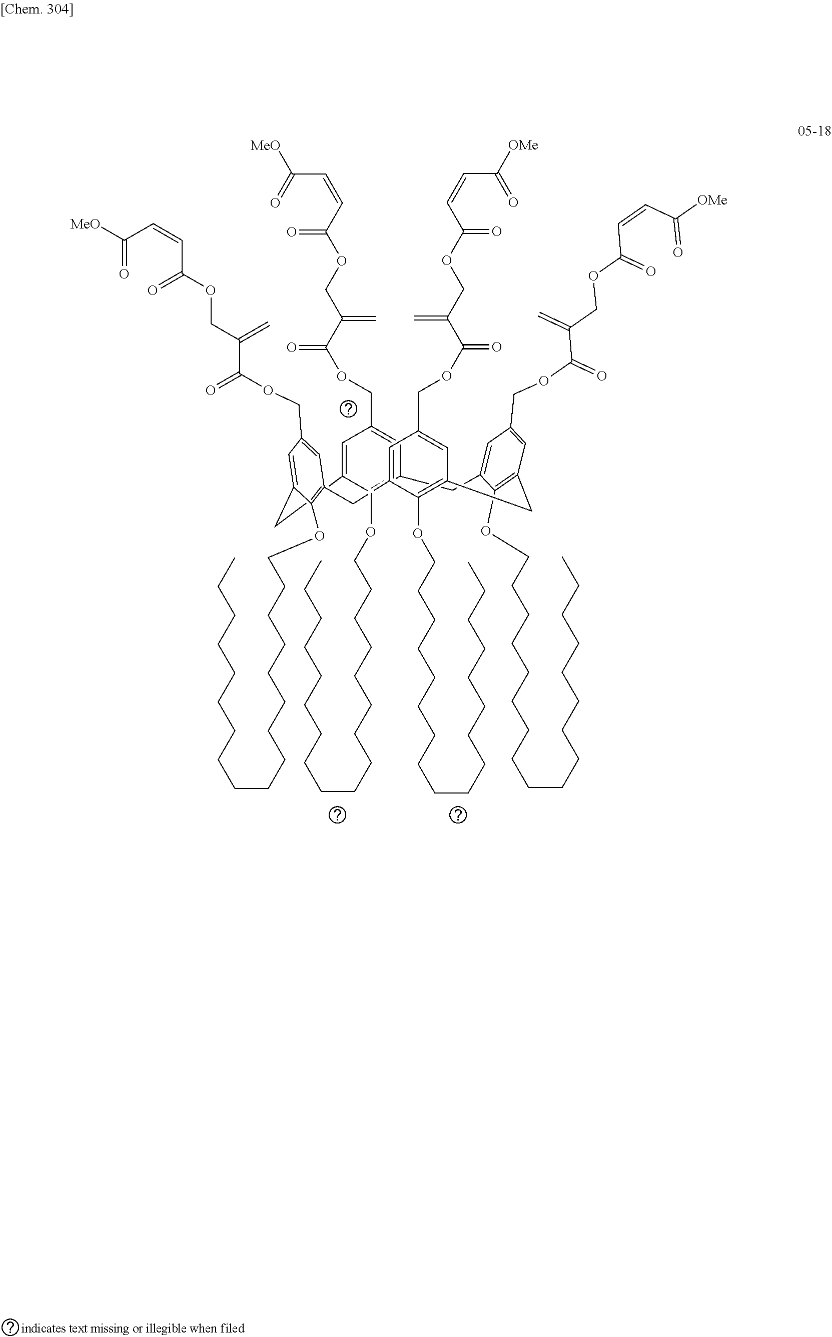 [SMILES: C=C(COC(=O)/C=C\C(=O)OC)C(=O)OCc1cc2c(OCCCCCCCCCCCCCCCCCC)c(c1)Cc1cc(COC(=O)C(=C)COC(=O)/C=C\C(=O)OC)cc(c1OCCCCCCCCCCCCCCCCCC)Cc1cc(COC(=O)C(=C)COC(=O)/C=C\C(=O)OC)cc(c1OCCCCCCCCCCCCCCCCCC)Cc1cc(COC(=O)C(=C)COC(=O)/C=C\C(=O)OC)cc(c1OCCCCCCCCCCCCCCCCCC)C2]

### Example 45

A 100 mL four-necked flask equipped with a stirrer, a dropping funnel, and a thermometer was charged, in a nitrogen atmosphere, with the compound obtained in Synthesis Example 59 (3.0 g, 3.02 mmol), triphenylphosphine (4.752 g, 18.12 mmol), acrylic acid (0.653 g, 9.06 mmol), monomethyl maleate (1.179, 9.06 mmol), and 46.0 mL of tetrahydrofuran, and the mixture was stirred. Then diisopropyl azodicarboxylate (3.664 g, 18.12 mmol) was added dropwise to the mixture placed in an ice bath over 30 minutes, and the resulting mixture was further stirred at room temperature for 24 hours. The reaction solution was concentrated in an evaporator, and hexane was added to precipitate and remove by-products such as triphenylphosphine. The obtained yellow viscous liquid was purified by silica gel column chromatography, and the target compounds 06-6, 07-6, 08-6, and 09-6 were obtained as follows. 06-6 (0.577 g, yield 13.8%). A mixture of 07-6 and 08-6 (2.138 g, yield 53.4%). 09-6 (0.494 g, yield 12.9%).

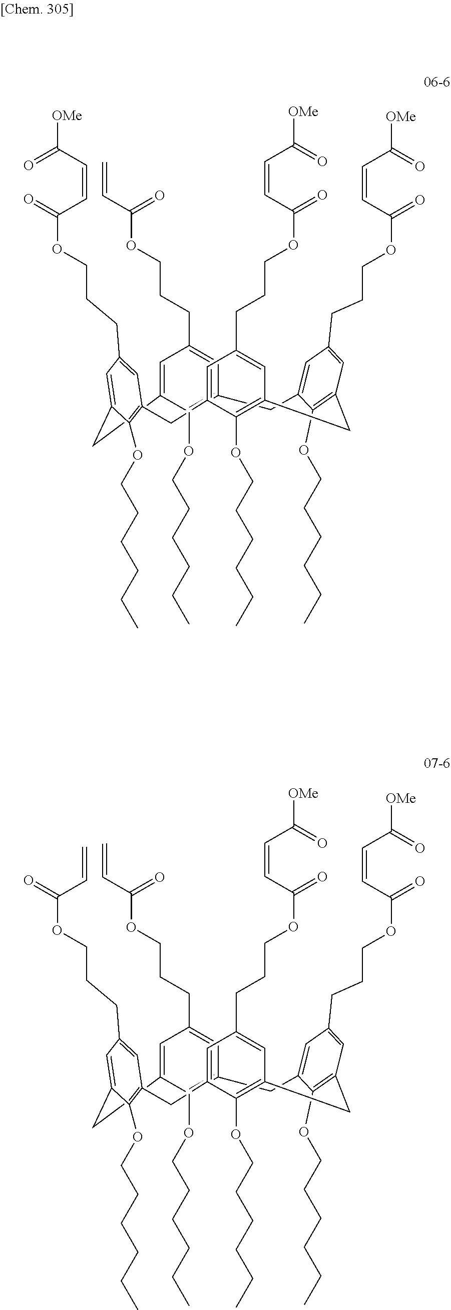 [SMILES: C=CC(=O)OCCCc1cc2c(OCCCCCC)c(c1)Cc1cc(CCCOC(=O)/C=C\C(=O)OC)cc(c1OCCCCCC)Cc1cc(CCCOC(=O)/C=C\C(=O)OC)cc(c1OCCCCCC)Cc1cc(CCCOC(=O)/C=C\C(=O)OC)cc(c1OCCCCCC)C2 ; C=CC(=O)OCCCc1cc2c(OCCCCCC)c(c1)Cc1cc(CCCOC(=O)/C=C\C(=O)OC)cc(c1OCCCCCC)Cc1cc(CCCOC(=O)/C=C\C(=O)OC)cc(c1OCCCCCC)Cc1cc(CCCOC(=O)C=C)cc(c1OCCCCCC)C2]

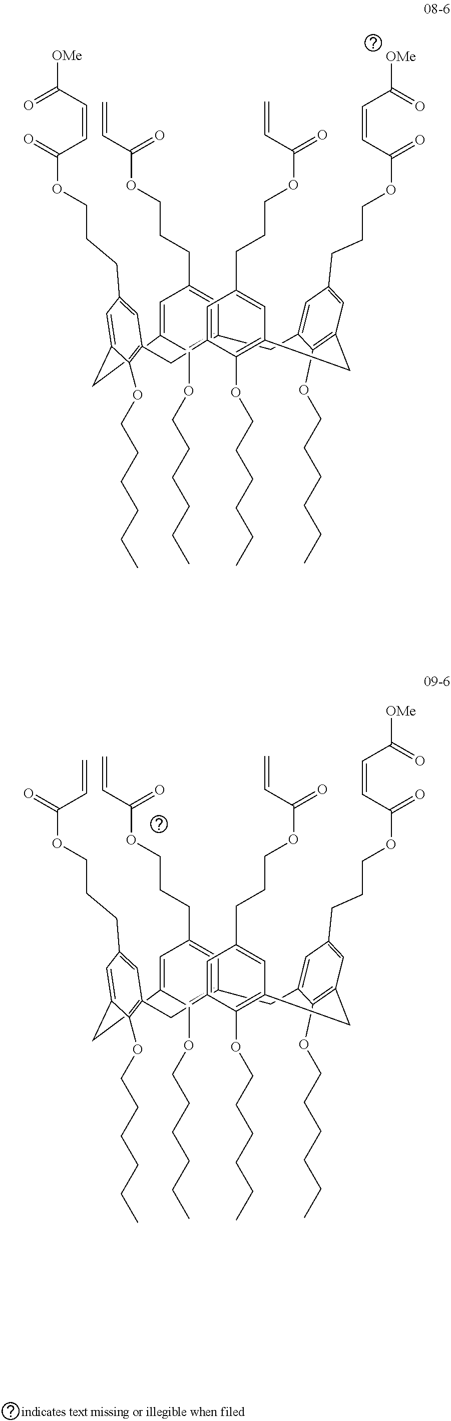 [SMILES: C=CC(=O)OCCCc1cc2c(OCCCCCC)c(c1)Cc1cc(CCCOC(=O)/C=C\C(=O)OC)cc(c1OCCCCCC)Cc1cc(CCCOC(=O)C=C)cc(c1OCCCCCC)Cc1cc(CCCOC(=O)/C=C\C(=O)OC)cc(c1OCCCCCC)C2 ; C=CC(=O)OCCCc1cc2c(OCCCCCC)c(c1)Cc1cc(CCCOC(=O)C=C)cc(c1OCCCCCC)Cc1cc(CCCOC(=O)/C=C\C(=O)OC)cc(c1OCCCCCC)Cc1cc(CCCOC(=O)C=C)cc(c1OCCCCCC)C2]

### Example 46

The same procedure as in Example 45 was repeated except that the compound obtained in Synthesis Example 60 (3.00 g, 4.21 mmol) was used instead of the compound obtained in Synthesis Example 59, to thereby obtain the target compounds 06-1, 07-1, 08-1, and 09-1 as follows. 06-1 (0.594 g, yield 12.8%). A mixture of 07-1 and 08-1 (2.406 g, yield 54.7%). 09-1 (0.548 g, yield 13.2%).

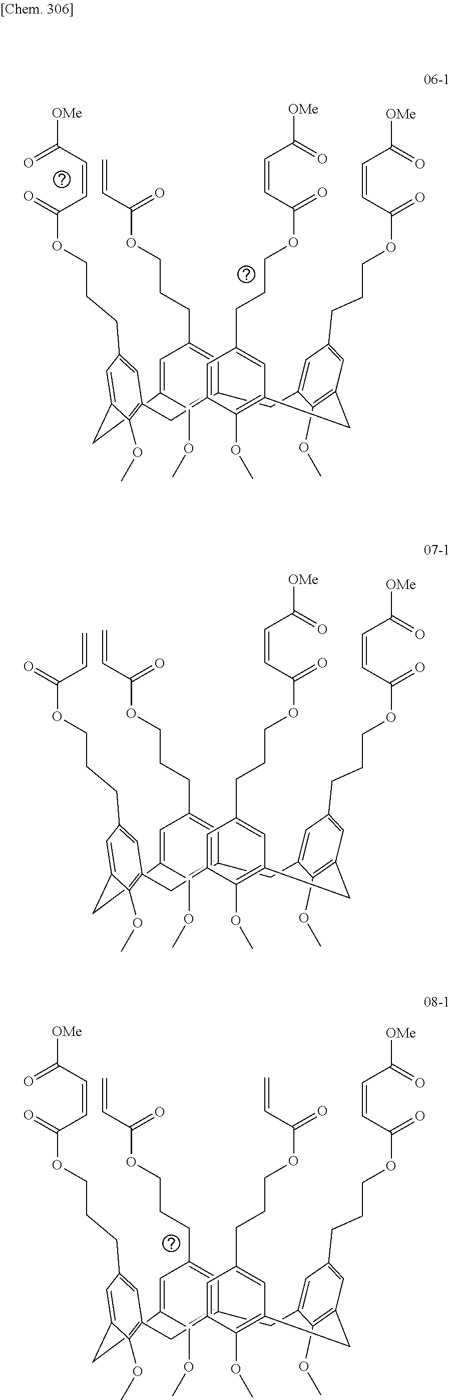 [SMILES: C=CC(=O)OCCCc1cc2c(OC)c(c1)Cc1cc(CCCOC(=O)/C=C\C(=O)OC)cc(c1OC)Cc1cc(CCCOC(=O)/C=C\C(=O)OC)cc(c1OC)Cc1cc(CCCOC(=O)/C=C\C(=O)OC)cc(c1OC)C2 ; C=CC(=O)OCCCc1cc2c(OC)c(c1)Cc1cc(CCCOC(=O)/C=C\C(=O)OC)cc(c1OC)Cc1cc(CCCOC(=O)/C=C\C(=O)OC)cc(c1OC)Cc1cc(CCCOC(=O)C=C)cc(c1OC)C2 ; C=CC(=O)OCCCc1cc2c(OC)c(c1)Cc1cc(CCCOC(=O)/C=C\C(=O)OC)cc(c1OC)Cc1cc(CCCOC(=O)C=C)cc(c1OC)Cc1cc(CCCOC(=O)/C=C\C(=O)OC)cc(c1OC)C2]

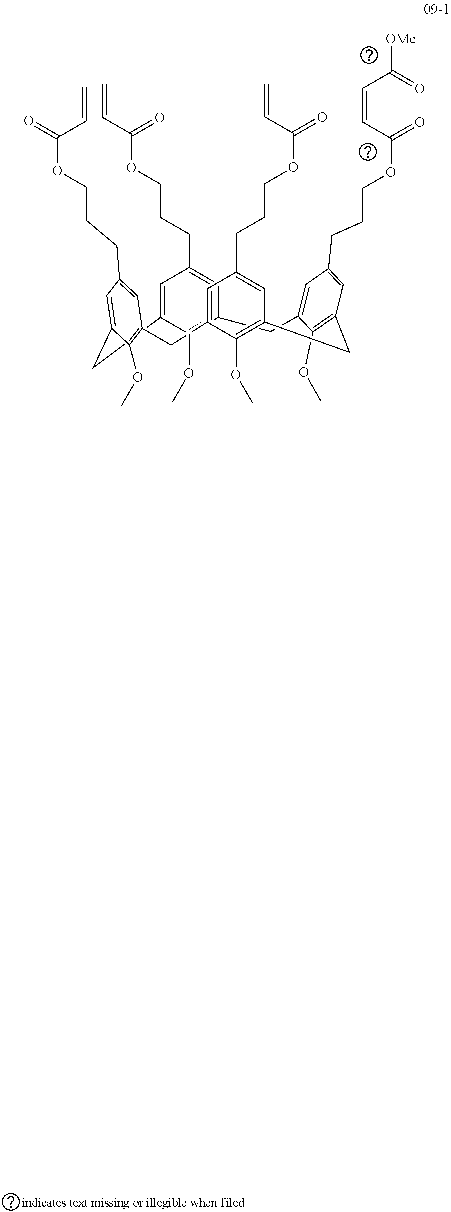 [SMILES: C=CC(=O)OCCCc1cc2c(OC)c(c1)Cc1cc(CCCOC(=O)C=C)cc(c1OC)Cc1cc(CCCOC(=O)/C=C\C(=O)OC)cc(c1OC)Cc1cc(CCCOC(=O)C=C)cc(c1OC)C2]

### Example 47

The same procedure as in Example 45 was repeated except that the compound obtained in Synthesis Example 61 (3.00 g, 3.40 mmol) was used instead of the compound obtained in Synthesis Example 59, to thereby obtain the target compounds 06-4, 07-4, 08-4, and 09-4 as follows. 06-4 (0.602 g, yield 13.9%). A mixture of 07-1 and 08-1 (2.185 g, yield 52.9%). 09-4 (0.622 g, yield 15.8%).

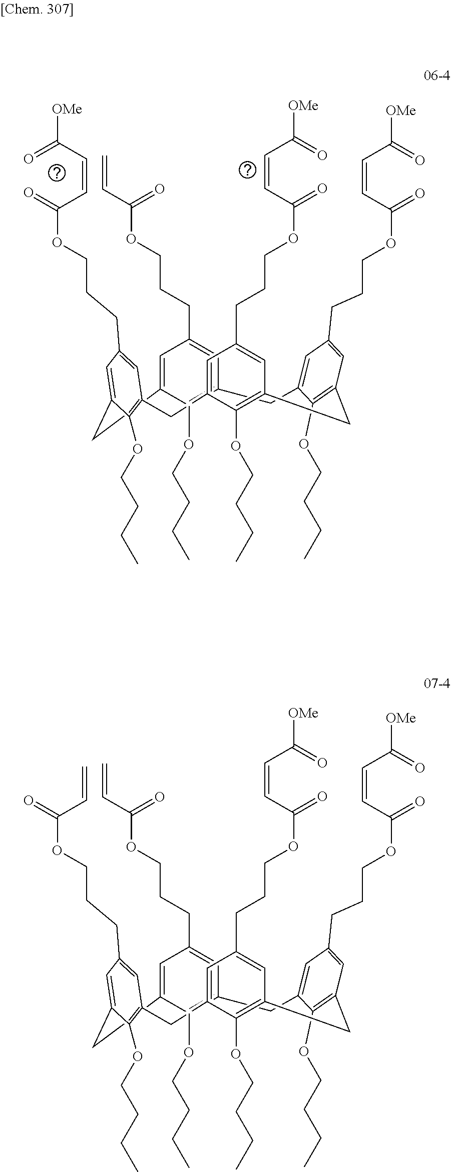 [SMILES: C=CC(=O)OCCCc1cc2c(OCCCC)c(c1)Cc1cc(CCCOC(=O)/C=C\C(=O)OC)cc(c1OCCCC)Cc1cc(CCCOC(=O)/C=C\C(=O)OC)cc(c1OCCCC)Cc1cc(CCCOC(=O)/C=C\C(=O)OC)cc(c1OCCCC)C2 ; C=CC(=O)OCCCc1cc2c(OCCCC)c(c1)Cc1cc(CCCOC(=O)/C=C\C(=O)OC)cc(c1OCCCC)Cc1cc(CCCOC(=O)/C=C\C(=O)OC)cc(c1OCCCC)Cc1cc(CCCOC(=O)C=C)cc(c1OCCCC)C2]

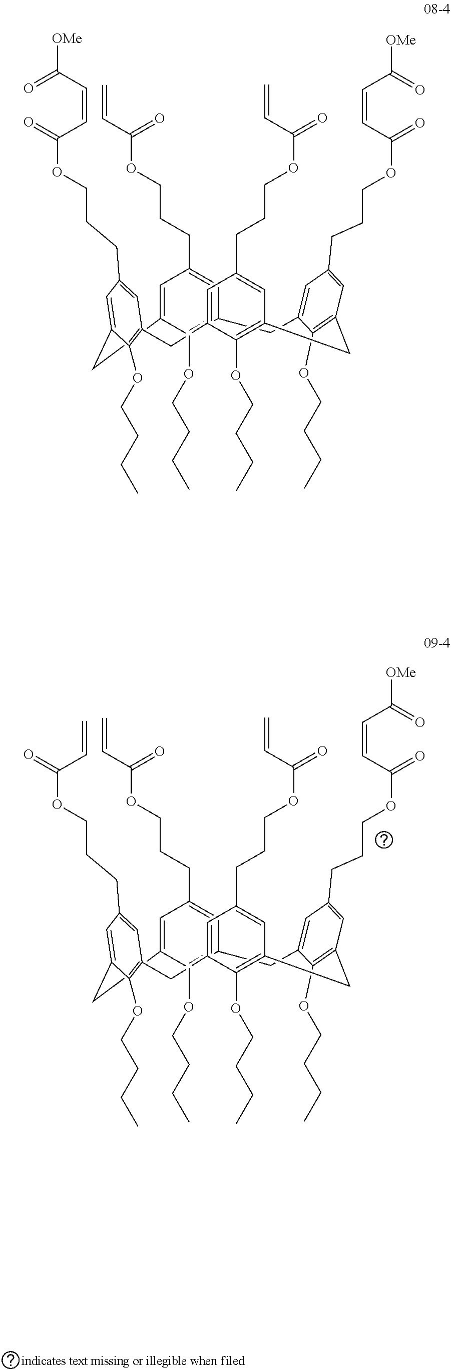 [SMILES: C=CC(=O)OCCCc1cc2c(OCCCC)c(c1)Cc1cc(CCCOC(=O)/C=C\C(=O)OC)cc(c1OCCCC)Cc1cc(CCCOC(=O)C=C)cc(c1OCCCC)Cc1cc(CCCOC(=O)/C=C\C(=O)OC)cc(c1OCCCC)C2 ; C=CC(=O)OCCCc1cc2c(OCCCC)c(c1)Cc1cc(CCCOC(=O)C=C)cc(c1OCCCC)Cc1cc(CCCOC(=O)/C=C\C(=O)OC)cc(c1OCCCC)Cc1cc(CCCOC(=O)C=C)cc(c1OCCCC)C2]

### Example 48

The same procedure as in Example 45 was repeated except that the compound obtained in Synthesis Example 62 (3.00 g, 2.86 mmol) was used instead of the compound obtained in Synthesis Example 59, to thereby obtain the target compounds 06-7, 07-7, 08-7, and 09-7 as follows. 06-7 (0.597 g, yield 14.5%). A mixture of 07-7 and 08-7 (2.117 g, yield 53.6%) 09-7 (0.469 g, yield 12.4%).

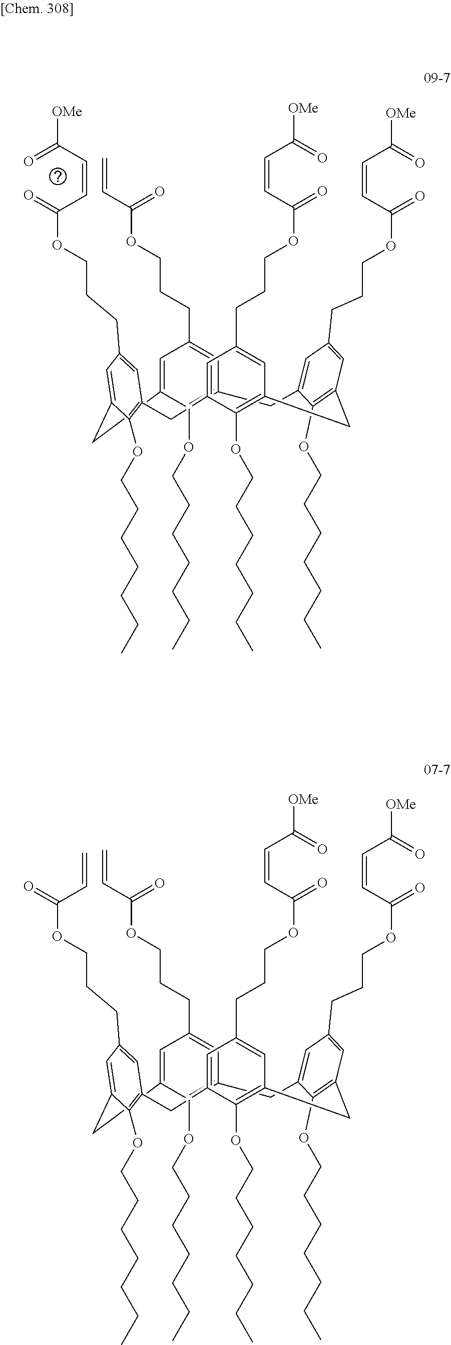 [SMILES: C=CC(=O)OCCCc1cc2c(OCCCCCCC)c(c1)Cc1cc(CCCOC(=O)/C=C\C(=O)OC)cc(c1OCCCCCCC)Cc1cc(CCCOC(=O)/C=C\C(=O)OC)cc(c1OCCCCCCC)Cc1cc(CCCOC(=O)/C=C\C(=O)OC)cc(c1OCCCCCCC)C2 ; C=CC(=O)OCCCc1cc2c(OCCCCCCC)c(c1)Cc1cc(CCCOC(=O)/C=C\C(=O)OC)cc(c1OCCCCCCC)Cc1cc(CCCOC(=O)/C=C\C(=O)OC)cc(c1OCCCCCCC)Cc1cc(CCCOC(=O)C=C)cc(c1OCCCCCCC)C2]

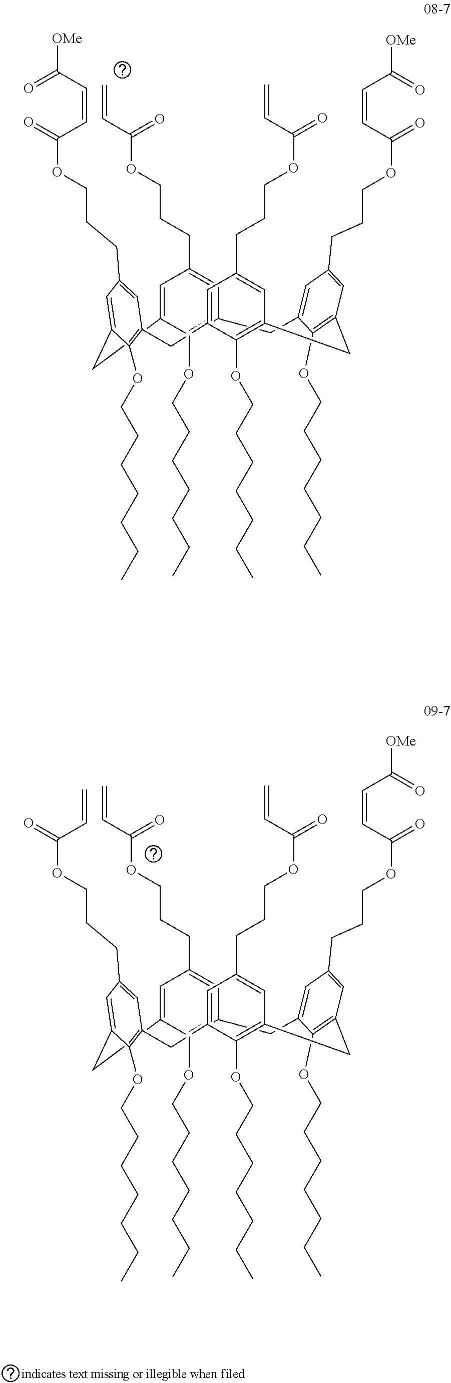 [SMILES: C=CC(=O)OCCCc1cc2c(OCCCCCCC)c(c1)Cc1cc(CCCOC(=O)/C=C\C(=O)OC)cc(c1OCCCCCCC)Cc1cc(CCCOC(=O)C=C)cc(c1OCCCCCCC)Cc1cc(CCCOC(=O)/C=C\C(=O)OC)cc(c1OCCCCCCC)C2 ; C=CC(=O)OCCCc1cc2c(OCCCCCCC)c(c1)Cc1cc(CCCOC(=O)C=C)cc(c1OCCCCCCC)Cc1cc(CCCOC(=O)/C=C\C(=O)OC)cc(c1OCCCCCCC)Cc1cc(CCCOC(=O)C=C)cc(c1OCCCCCCC)C2]

### Example 49

The same procedure as in Example 45 was repeated except that the compound obtained in Synthesis Example 63 (3.00 g, 1.80 mmol) was used instead of the compound obtained in Synthesis Example 59, to thereby obtain the target compounds 06-18, 07-18, 08-18, and 09-18 as follows. 06-18 (0.50 g, yield 13.5%). A mixture of 07-18 and 08-18 (1.857 g, yield 51.6%). 09-18 (0.444 g, yield 12.7%).

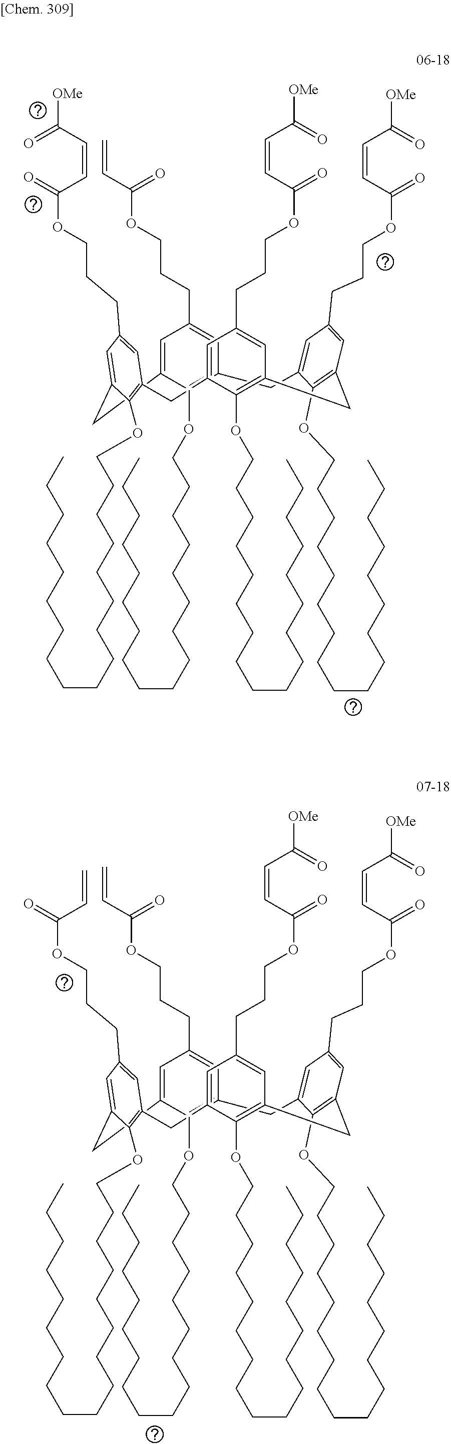 [SMILES: C=CC(=O)OCCCc1cc2c(OCCCCCCCCCCCCCCCCCC)c(c1)Cc1cc(CCCOC(=O)/C=C\C(=O)OC)cc(c1OCCCCCCCCCCCCCCCCCC)Cc1cc(CCCOC(=O)/C=C\C(=O)OC)cc(c1OCCCCCCCCCCCCCCCCCC)Cc1cc(CCCOC(=O)/C=C\C(=O)OC)cc(c1OCCCCCCCCCCCCCCCCCC)C2 ; C=CC(=O)OCCCc1cc2c(OCCCCCCCCCCCCCCCCCC)c(c1)Cc1cc(CCCOC(=O)/C=C\C(=O)OC)cc(c1OCCCCCCCCCCCCCCCCCC)Cc1cc(CCCOC(=O)/C=C\C(=O)OC)cc(c1OCCCCCCCCCCCCCCCCCC)Cc1cc(CCCOC(=O)C=C)cc(c1OCCCCCCCCCCCCCCCCCC)C2]

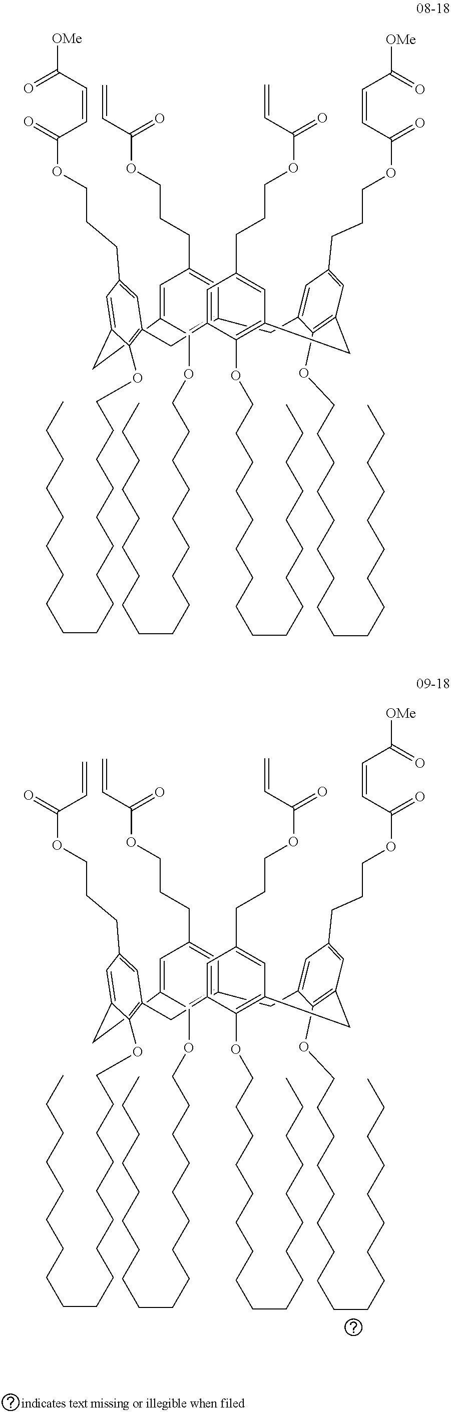 [SMILES: C=CC(=O)OCCCc1cc2c(OCCCCCCCCCCCCCCCCCC)c(c1)Cc1cc(CCCOC(=O)/C=C\C(=O)OC)cc(c1OCCCCCCCCCCCCCCCCCC)Cc1cc(CCCOC(=O)C=C)cc(c1OCCCCCCCCCCCCCCCCCC)Cc1cc(CCCOC(=O)/C=C\C(=O)OC)cc(c1OCCCCCCCCCCCCCCCCCC)C2 ; C=CC(=O)OCCCc1cc2c(OCCCCCCCCCCCCCCCCCC)c(c1)Cc1cc(CCCOC(=O)C=C)cc(c1OCCCCCCCCCCCCCCCCCC)Cc1cc(CCCOC(=O)/C=C\C(=O)OC)cc(c1OCCCCCCCCCCCCCCCCCC)Cc1cc(CCCOC(=O)C=C)cc(c1OCCCCCCCCCCCCCCCCCC)C2]

### Synthesis Example 74

A 100 mL four-necked flask equipped with a stirrer, a thermometer, and a reflux condenser tube was charged with 2.50 g (2.52 mmol) of the compound obtained in Synthesis Example 59, 3.96 g (15.10 mmol) of triphenylphosphine, 3.267 g (15.10 mmol) of 2-[[[(1,1-dimethylethyl)dimethylsilyl]oxy]2-propenoic acid, and 43 mL of tetrahydrofuran, and the mixture was stirred. Then 3.053 g (15.10 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 3.251 g, yield 72.3%).

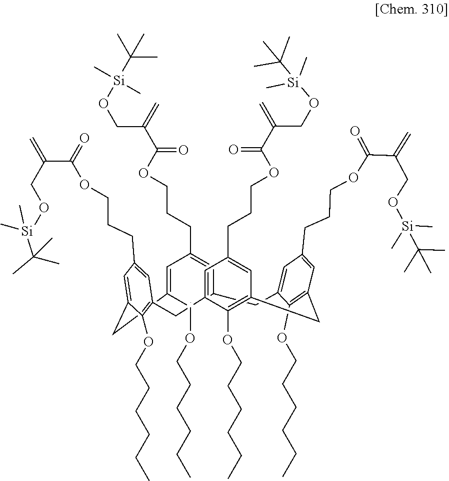 [SMILES: C=C(CO[Si](C)(C)C(C)(C)C)C(=O)OCCCc1cc2c(OCCCCCC)c(c1)Cc1cc(CCCOC(=O)C(=C)CO[Si](C)(C)C(C)(C)C)cc(c1OCCCCCC)Cc1cc(CCCOC(=O)C(=C)CO[Si](C)(C)C(C)(C)C)cc(c1OCCCCCC)Cc1cc(CCCOC(=O)C(=C)CO[Si](C)(C)C(C)(C)C)cc(c1OCCCCCC)C2]

### Synthesis Example 75

The same procedure as in Synthesis Example 74 was repeated except that the compound obtained in Synthesis Example 60 (2.50 g, 3.33 mmol) was used instead of compound obtained in Synthesis Example 59, to thereby obtain a compound represented by the following formula (3.782 g, yield 71.6%).

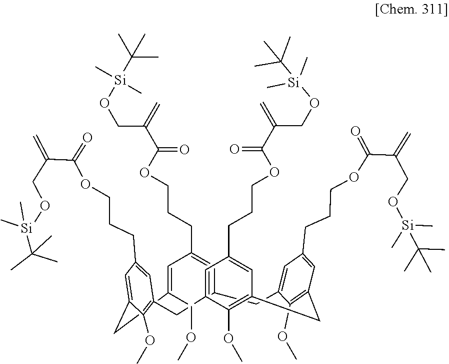 [SMILES: C=C(CO[Si](C)(C)C(C)(C)C)C(=O)OCCCc1cc2c(OC)c(c1)Cc1cc(CCCOC(=O)C(=C)CO[Si](C)(C)C(C)(C)C)cc(c1OC)Cc1cc(CCCOC(=O)C(=C)CO[Si](C)(C)C(C)(C)C)cc(c1OC)Cc1cc(CCCOC(=O)C(=C)CO[Si](C)(C)C(C)(C)C)cc(c1OC)C2]

### Synthesis Example 76

The same procedure as in Synthesis Example 74 was repeated except that the compound obtained in Synthesis Example 61 (2.50 g, 2.84 mmol) was used instead of the compound obtained in Synthesis Example 59, to thereby obtain a compound represented by the following formula (3.553 g, yield 74.8%).

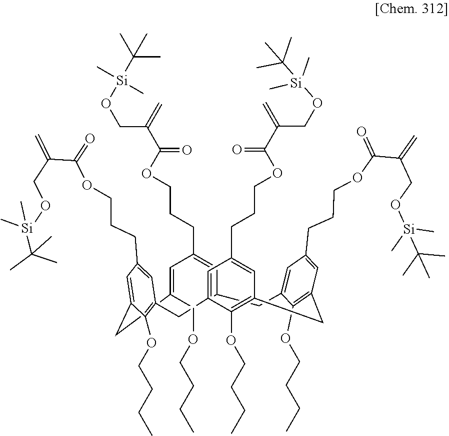 [SMILES: C=C(CO[Si](C)(C)C(C)(C)C)C(=O)OCCCc1cc2c(OCCCC)c(c1)Cc1cc(CCCOC(=O)C(=C)CO[Si](C)(C)C(C)(C)C)cc(c1OCCCC)Cc1cc(CCCOC(=O)C(=C)CO[Si](C)(C)C(C)(C)C)cc(c1OCCCC)Cc1cc(CCCOC(=O)C(=C)CO[Si](C)(C)C(C)(C)C)cc(c1OCCCC)C2]

### Synthesis Example 77

The same procedure as in Synthesis Example 74 was repeated except that the compound obtained in Synthesis Example 62 (2.50 g, 2.38 mmol) was used instead of the compound obtained in Synthesis Example 59, to thereby obtain a compound represented by the following formula (3.305 g, yield 75.3%).

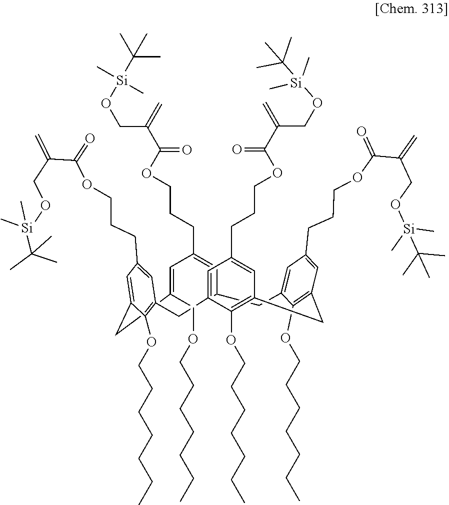 [SMILES: C=C(CO[Si](C)(C)C(C)(C)C)C(=O)OCCCc1cc2c(OCCCCCCC)c(c1)Cc1cc(CCCOC(=O)C(=C)CO[Si](C)(C)C(C)(C)C)cc(c1OCCCCCCC)Cc1cc(CCCOC(=O)C(=C)CO[Si](C)(C)C(C)(C)C)cc(c1OCCCCCCC)Cc1cc(CCCOC(=O)C(=C)CO[Si](C)(C)C(C)(C)C)cc(c1OCCCCCCC)C2]

### Synthesis Example 78

The same procedure as in Synthesis Example 74 was repeated except that the compound obtained in Synthesis Example 63 (2.50 g, 1.50 mmol) was used instead of the compound obtained in Synthesis Example 59, to thereby obtain a compound represented by the following formula (3.011 g, yield 81.6%).

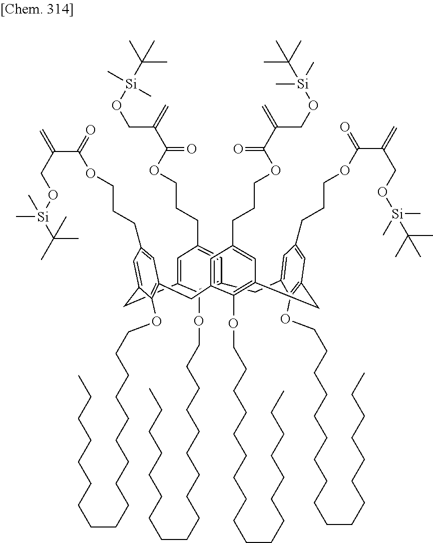 [SMILES: C=C(CO[Si](C)(C)C(C)(C)C)C(=O)OCCCc1cc2c(OCCCCCCCCCCCCCCCCCC)c(c1)Cc1cc(CCCOC(=O)C(=C)CO[Si](C)(C)C(C)(C)C)cc(c1OCCCCCCCCCCCCCCCCCC)Cc1cc(CCCOC(=O)C(=C)CO[Si](C)(C)C(C)(C)C)cc(c1OCCCCCCCCCCCCCCCCCC)Cc1cc(CCCOC(=O)C(=C)CO[Si](C)(C)C(C)(C)C)cc(c1OCCCCCCCCCCCCCCCCCC)C2]

### Synthesis Example 79

A 200 mL four-necked flask equipped with a stirrer, a thermometer, and a reflux condenser tube was charged with 3.50 g (1.96 mmol) of the compound obtained in Synthesis Example 74, 0.706 g (11.75 mmol) of acetic acid, and 78.4 mL of tetrahydrofuran, and the mixture was stirred. A colorless transparent solution. Then tetrabutylammonium fluoride (an about 1 mol/L tetrahydrofuran solution 11.75 mL (11.75 mmol) was slowly added dropwise to the mixture placed in an ice bath under stirring. The mixture was stirred at room temperature for 12 hours. A saturated aqueous ammonium chloride solution was added to the reaction mixture, and then 50 mL of chloroform was added. The reaction mixture was transferred to a separatory funnel, and the organic layer was separated. Then the aqueous layer was extracted with 50 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula (yield amount 2.417 g, yield 92.8%).

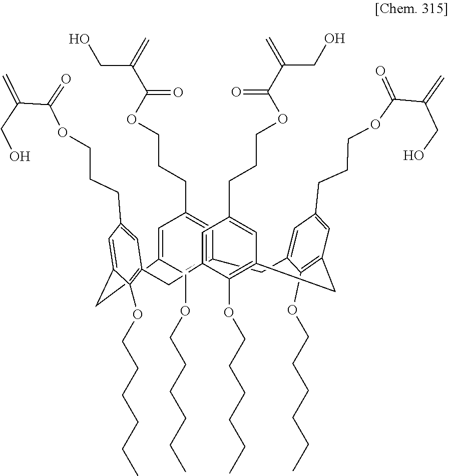 [SMILES: C=C(CO)C(=O)OCCCc1cc2c(OCCCCCC)c(c1)Cc1cc(CCCOC(=O)C(=C)CO)cc(c1OCCCCCC)Cc1cc(CCCOC(=O)C(=C)CO)cc(c1OCCCCCC)Cc1cc(CCCOC(=O)C(=C)CO)cc(c1OCCCCCC)C2]

### Synthesis Example 80

The same procedure as in Synthesis Example 79 was repeated except that the compound obtained in Synthesis Example 75 (3.50 g, 2.32 mmol) was used instead of the compound obtained in Synthesis Example 74, to thereby obtain a compound represented by the following formula (2.214 g, yield 90.8%).

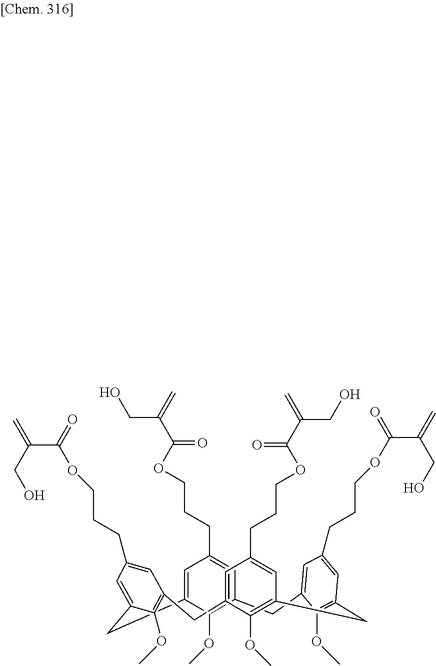 [SMILES: C=C(CO)C(=O)OCCCc1cc2c(OC)c(c1)Cc1cc(CCCOC(=O)C(=C)CO)cc(c1OC)Cc1cc(CCCOC(=O)C(=C)CO)cc(c1OC)Cc1cc(CCCOC(=O)C(=C)CO)cc(c1OC)C2]

### Synthesis Example 81

The same procedure as in Synthesis Example 79 was repeated except that the compound obtained in Synthesis Example 76 (3.50 g, 2.32 mmol) was used instead of the compound obtained in Synthesis Example 74, to thereby obtain a compound represented by the following formula (2.344 g, yield 92.1%).

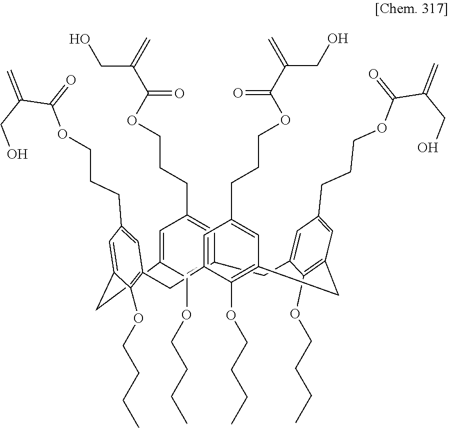 [SMILES: C=C(CO)C(=O)OCCCc1cc2c(OCCCC)c(c1)Cc1cc(CCCOC(=O)C(=C)CO)cc(c1OCCCC)Cc1cc(CCCOC(=O)C(=C)CO)cc(c1OCCCC)Cc1cc(CCCOC(=O)C(=C)CO)cc(c1OCCCC)C2]

### Synthesis Example 82

The same procedure as in Synthesis Example 79 was repeated except that the compound obtained in Synthesis Example 77 (3.50 g, 2.32 mmol) was used instead of the compound obtained in Synthesis Example 74, to thereby obtain a compound represented by the following formula (2.466 g, yield 93.7%).

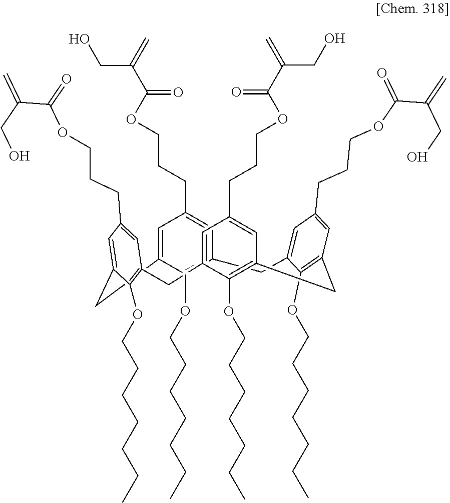 [SMILES: C=C(CO)C(=O)OCCCc1cc2c(OCCCCCCC)c(c1)Cc1cc(CCCOC(=O)C(=C)CO)cc(c1OCCCCCCC)Cc1cc(CCCOC(=O)C(=C)CO)cc(c1OCCCCCCC)Cc1cc(CCCOC(=O)C(=C)CO)cc(c1OCCCCCCC)C2]

### Synthesis Example 83

The same procedure as in Synthesis Example 79 was repeated except that the compound obtained in Synthesis Example 78 (3.50 g, 1.42 mmol) was used instead of the compound obtained in Synthesis Example 74, to thereby obtain a compound represented by the following formula (2.608 g, yield 91.5%).

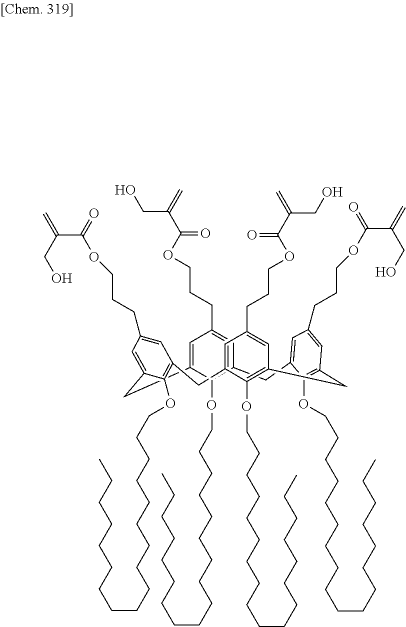 [SMILES: C=C(CO)C(=O)OCCCc1cc2c(OCCCCCCCCCCCCCCCCCC)c(c1)Cc1cc(CCCOC(=O)C(=C)CO)cc(c1OCCCCCCCCCCCCCCCCCC)Cc1cc(CCCOC(=O)C(=C)CO)cc(c1OCCCCCCCCCCCCCCCCCC)Cc1cc(CCCOC(=O)C(=C)CO)cc(c1OCCCCCCCCCCCCCCCCCC)C2]

### Example 50

A 100 mL four-necked flask equipped with a stirrer, a thermometer, and a reflux condenser tube was charged with 2.00 g (1.50 mmol) of the compound obtained in Synthesis Example 79, triphenylphosphine (2.367 g, 9.02 mmol), monomethyl maleate (1.174 g, 9.02 mmol), and 24.8 mL of tetrahydrofuran, and the mixture was stirred. Then diisopropyl azodicarboxylate (1.825 g, 9.02 mmol) was added dropwise to the mixture placed in an ice bath over 30 minutes, and the resulting mixture was further stirred at room temperature for 24 hours. The reaction solution was concentrated in an evaporator, and hexane was added to precipitate and remove by-products such as triphenylphosphine. The obtained yellow viscous liquid was purified by silica gel column chromatography to thereby obtain the target compound 10-6 (yield amount 2.340 g, yield 87.5%).

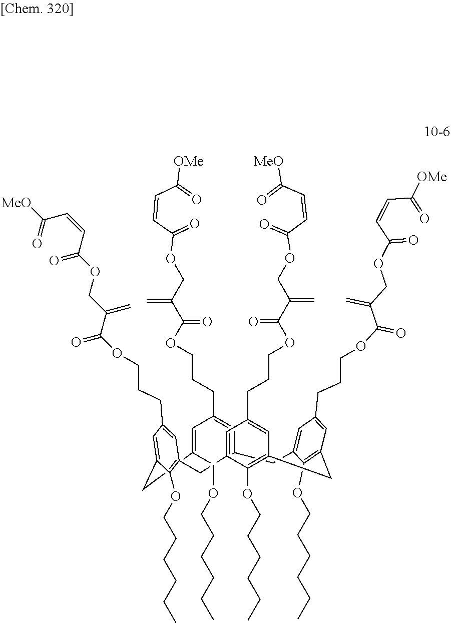 [SMILES: C=C(COC(=O)/C=C\C(=O)OC)C(=O)OCCCc1cc2c(OCCCCCC)c(c1)Cc1cc(CCCOC(=O)C(=C)COC(=O)/C=C\C(=O)OC)cc(c1OCCCCCC)Cc1cc(CCCOC(=O)C(=C)COC(=O)/C=C\C(=O)OC)cc(c1OCCCCCC)Cc1cc(CCCOC(=O)C(=C)COC(=O)/C=C\C(=O)OC)cc(c1OCCCCCC)C2]

### Example 51

The same procedure as in Example 50 was repeated except that the compound obtained in Synthesis Example 80 (2.00 g, 1.91 mmol) was used instead of the compound obtained in Synthesis Example 79, to thereby obtain the target compound 10-1 (2.432 g, yield 85.2%).

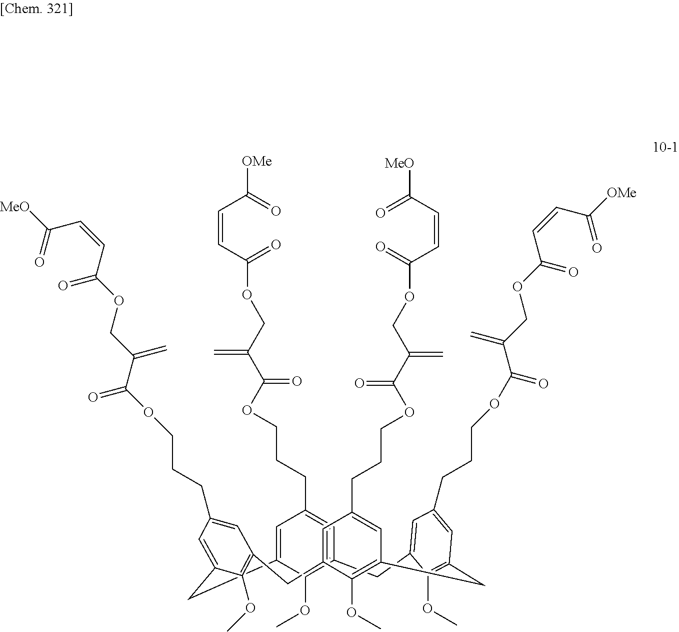 [SMILES: C=C(COC(=O)/C=C\C(=O)OC)C(=O)OCCCc1cc2c(OC)c(c1)Cc1cc(CCCOC(=O)C(=C)COC(=O)/C=C\C(=O)OC)cc(c1OC)Cc1cc(CCCOC(=O)C(=C)COC(=O)/C=C\C(=O)OC)cc(c1OC)Cc1cc(CCCOC(=O)C(=C)COC(=O)/C=C\C(=O)OC)cc(c1OC)C2]

### Example 52

The same procedure as in Example 50 was repeated except that the compound obtained in Synthesis Example 81 (2.00 g, 1.64 mmol) was used instead of the compound obtained in Synthesis Example 79, to thereby obtain the target compound 10-4 (2.375 g, yield 86.8%).

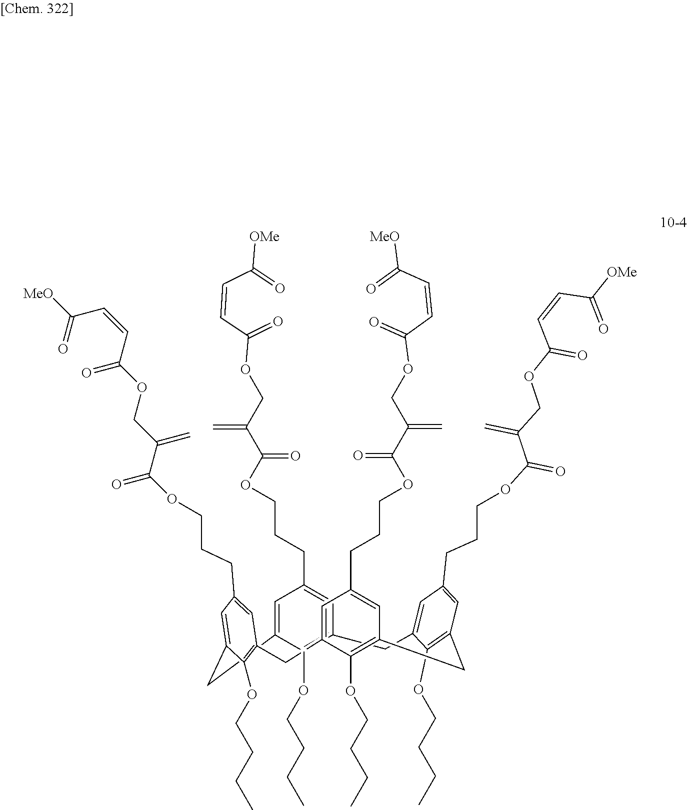 [SMILES: C=C(COC(=O)/C=C\C(=O)OC)C(=O)OCCCc1cc2c(OCCCC)c(c1)Cc1cc(CCCOC(=O)C(=C)COC(=O)/C=C\C(=O)OC)cc(c1OCCCC)Cc1cc(CCCOC(=O)C(=C)COC(=O)/C=C\C(=O)OC)cc(c1OCCCC)Cc1cc(CCCOC(=O)C(=C)COC(=O)/C=C\C(=O)OC)cc(c1OCCCC)C2]

### Example 53

The same procedure as in Example 50 was repeated except that the compound obtained in Synthesis Example 82 (2.00 g, 1.44 mmol) was used instead of the compound obtained in Synthesis Example 79, to thereby obtain the target compound 10-1 (2.417 g, yield 91.3%).

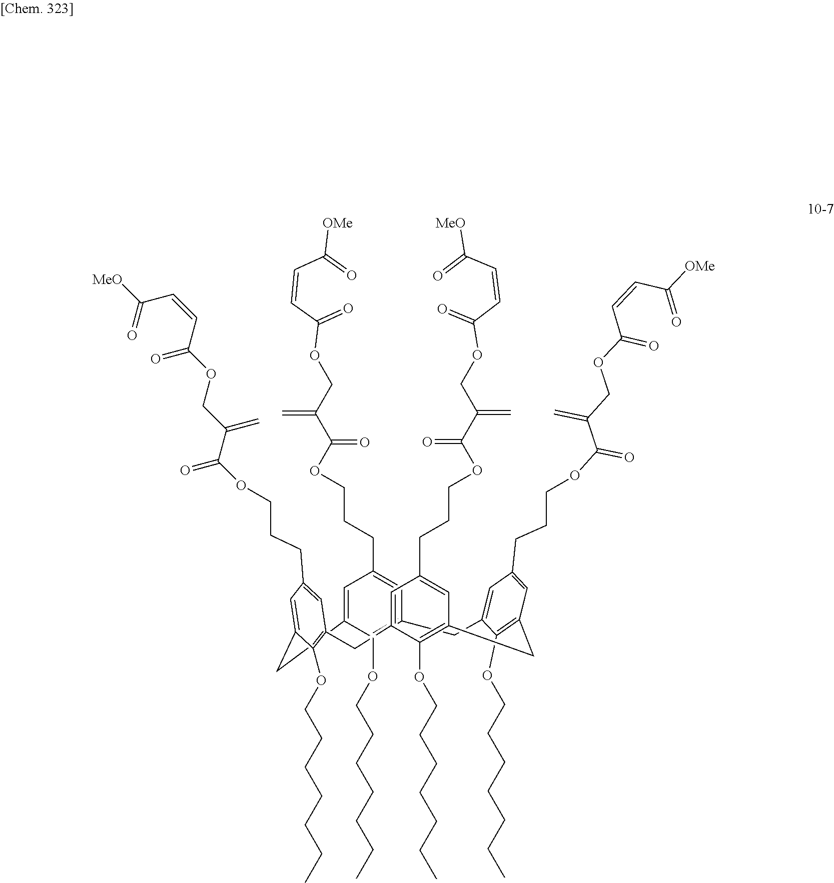 [SMILES: C=C(COC(=O)/C=C\C(=O)OC)C(=O)OCCCc1cc2c(OCCCCCCC)c(c1)Cc1cc(CCCOC(=O)C(=C)COC(=O)/C=C\C(=O)OC)cc(c1OCCCCCCC)Cc1cc(CCCOC(=O)C(=C)COC(=O)/C=C\C(=O)OC)cc(c1OCCCCCCC)Cc1cc(CCCOC(=O)C(=C)COC(=O)/C=C\C(=O)OC)cc(c1OCCCCCCC)C2]

### Example 54

The same procedure as in Example 50 was repeated except that the compound obtained in Synthesis Example 83 (2.00 g, 1.00 mmol) was used instead of the compound obtained in Synthesis Example 79, to thereby obtain the target compound 10-1 (1.961 g, yield 80.1%).

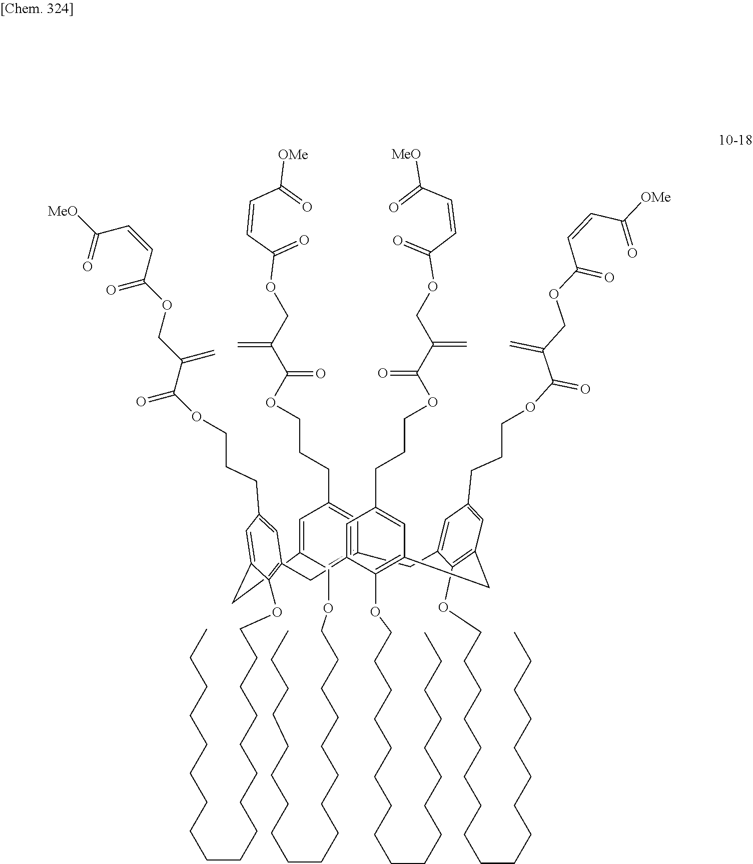 [SMILES: C=C(COC(=O)/C=C\C(=O)OC)C(=O)OCCCc1cc2c(OCCCCCCCCCCCCCCCCCC)c(c1)Cc1cc(CCCOC(=O)C(=C)COC(=O)/C=C\C(=O)OC)cc(c1OCCCCCCCCCCCCCCCCCC)Cc1cc(CCCOC(=O)C(=C)COC(=O)/C=C\C(=O)OC)cc(c1OCCCCCCCCCCCCCCCCCC)Cc1cc(CCCOC(=O)C(=C)COC(=O)/C=C\C(=O)OC)cc(c1OCCCCCCCCCCCCCCCCCC)C2]

### Comparative Example 1

A 100 mL four-necked flask equipped with a stirrer, a thermometer, and a reflux condenser tube was charged with 1.00 g (1.212 mmol) of I-6, 10.00 g (138.7 mmol) of tetrahydrofuran, 1.907 g (7.271 mmol) of triphenylphosphine, and 1.110 g (8.535 mmol) of monomethyl phthalate, and the mixture was stirred. A light yellow transparent solution. Then 1.470 g (7.271 mmol) of diisopropyl azodicarboxylate was added dropwise to the solution placed in an ice bath over 30 minutes. A light yellow transparent solution. The solution was stirred at room temperature for 6 hours. Hexane was added to the reaction solution to precipitate and remove by-products such as triphenylphosphine, and the resulting mixture was extracted with chloroform, washed with water and saturated brine, and dried over magnesium sulfate. The solvent was removed by evaporation using an evaporator, and the resulting orange viscus liquid was subjected to column chromatography (eluent: n-hexane:acetone=90:10) to thereby obtain compound 1′ represented by the following formula. Compound 1′ was vacuum dried (at 60° C. for 6 hours or longer). The yield amount was 1.331 g, and the yield was 72.5%.

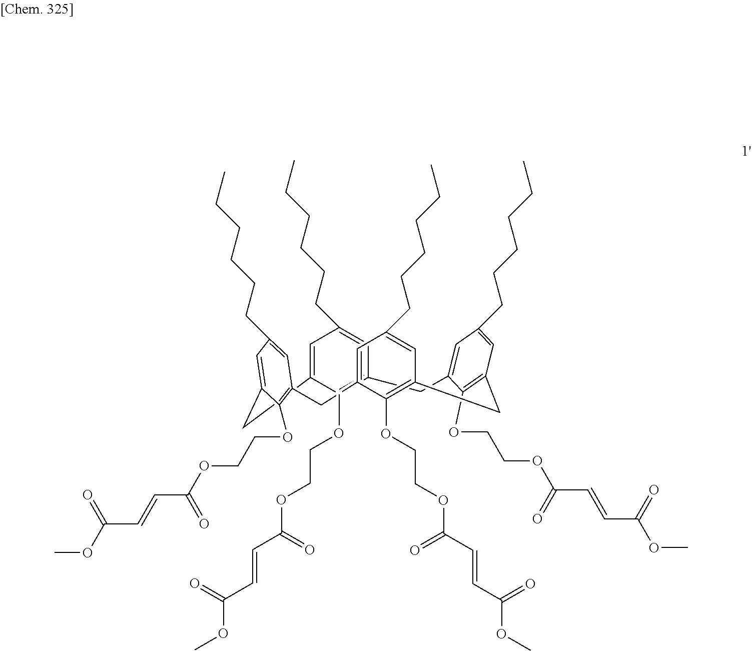

### Comparative Example 2

The same procedure as in Example 16 was repeated except that monomethyl phthalate was used instead of monomethyl maleate, to thereby obtain 0.434 g of compound 2′ represented by a formula below with a yield of 30.3%, 0.224 g of compound 3′ represented by a formula below with a yield of 16.2%, 0.209 g of compound 4′ represented by a formula below with a yield of 15.1%, and 0.139 g of compound 5′ represented by a formula below with a yield of 110.4%.

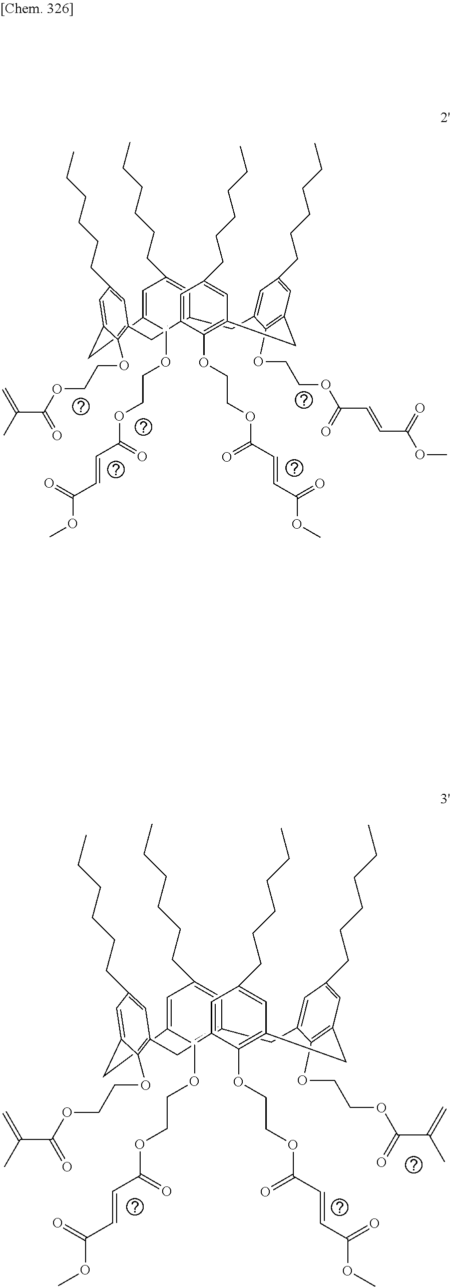

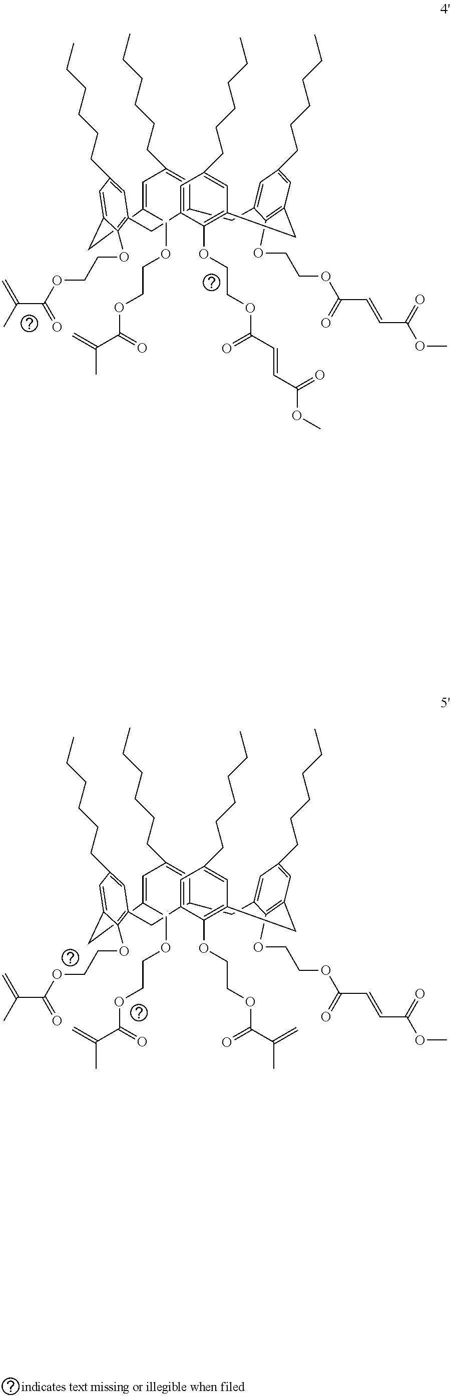

### Comparative Example 3

A 100 mL four-necked flask equipped with a stirrer, a thermometer, and a reflux condenser tube was charged with 1.00 g (1.212 mmol) of I-6, 10.00 g of tetrahydrofuran, 1.907 g (7.271 mmol) of triphenylphosphine, and 0.6260 g (7.271 mmol) of methacrylic acid, and the mixture was stirred. A light yellow transparent solution was thereby obtained. Then 1.470 g (7.271 mmol) of diisopropyl azodicarboxylate was added dropwise to the solution placed in an ice bath over 30 minutes. The light yellow transparent solution remained unchanged. The solution was stirred at room temperature for 6 hours. Hexane was added to the reaction solution to precipitate and remove by-products such as triphenylphosphine. The resulting solution was extracted with chloroform, washed with water and saturated brine, and dried over magnesium sulfate. The solvent was removed by evaporation using an evaporator, and the resulting orange viscus liquid was purified by column chromatography (eluent: n-hexane:acetone=90:10) to thereby obtain compound 6′ represented by the following formula. The yield amount was 0.9058 g, and the yield was 68.1%.

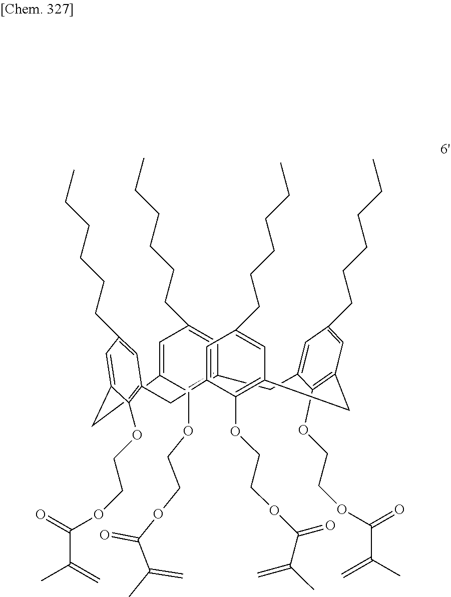 [SMILES: C=C(C)C(=O)OCCOc1c2cc(CCCCCC)cc1Cc1cc(CCCCCC)cc(c1OCCOC(=O)C(=C)C)Cc1cc(CCCCCC)cc(c1OCCOC(=O)C(=C)C)Cc1cc(CCCCCC)cc(c1OCCOC(=O)C(=C)C)C2]

<Production of Curable Compositions>

0.25 g of one of the obtained calixarene compounds, 0.25 g of dipentaerythritol hexaacrylate (“A-DPH” manufactured by Shin Nakamura Chemical Co., Ltd.), 0.005 g of a polymerization initiator (“Irgacure 369” manufactured by BASF), and 9.5 g of propylene glycol monomethyl ether acetate were mixed to obtain a curable composition.

<Production of Layered Bodies>

The curable composition was applied to substrates 1 to 4 below by a spin coating method such that the thickness of the coating after curing was about 0.5 μm and then dried on a hot plate at 100° C. for 2 minutes. A high-pressure mercury lamp was used to irradiate the curable composition with UV rays at 500 mJ/cm² in a nitrogen atmosphere to cure the curable composition, and layered bodies were thereby obtained.

Substrate 1: polymethyl methacrylate resin plate

Substrate 2: aluminum plate

Substrate 3: polyethylene terephthalate film having a SiO₂ thin layer (thickness 100 nm) (the curable composition was applied to the SiO₂ thin film)

<Evaluation of Adhesion>

A layered body stored in a 23° C. and 50% RH environment for 24 hours was used, and the adhesion was evaluated according to JIS K6500-5-6 (adhesive strength: a cross-cut method). A cellophane tape used was “CT-24” manufactured by Nichiban Co., Ltd. The criteria for the evaluation are as follows.

A: 80 or more out of 100 squares were not detached and remained present.

B: 50 to 79 out of 100 squares were not detached and remained present.

C: The number of squares that were not detached and remained present was 49 or less out of 100 squares.

<Evaluation of Moist Heat Resistance>

One of the curable compositions was applied to a 5 inch SiO substrate to a film thickness of about 50 μm using an applicator and dried on a hot plate at 100° C. for 2 minutes. A mask having an L/S pattern with L/S=50 μm/50 μm was brought into tight contact with the coating obtained, and a high-pressure mercury lamp was used to irradiate the composition with UV rays at 1000 mJ/cm² in a nitrogen atmosphere to cure the composition. The substrate exposed to the light was developed using ethyl acetate to obtain an evaluation substrate. The obtained substrate was stored in a thermo-hygrostat at 85° C. and 85% RH for 100 hours, and a laser microscope (“VK-X200” manufactured by KEYENCE CORPORATION) was used to check the state of the pattern after a lapse of 100 hours. The criteria for the evaluation are as follows.

A: The entire pattern was well modified and maintained.

B: Cracking or chipping was observed in part of the pattern.

C: Cracking or chipping was observed in the pattern, and delamination of the pattern was also observed.

TABLE 7

| Calixarene compound | | 1-6 | 2-6 | 3-6 | 4-6 | 1-4 | 2-4 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 3-4 | 4-4 | 1-7 | 2-7 | 3-7 | 4-7 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 5-6 | 6-6 | 7-6 | 8-6 | 9-6 | 10-6 |
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 8

| Calixarene compound | | 11-6 | 12-6 | 13-6 | 14-6 | 15-6 | 16-6 |
|---|---|---|---|---|---|---|---|
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 17-6 | 18-6 | 19-6 | 20-6 | 21-6 | 22-6 |
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | B |
| Calixarene compound | | 23-6 | 24-6 | 25-6 | 26-6 | 27-6 | 28-6 |
| Adhesion | Substrate 1 | A | 8 | A | A | A | 8 |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 9

| Calixarene compound | | 29-6 | 30-6 | 31-6 | 32-6 | 33-6 | 33-4 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 33-7 | 33-18 | 33-1 | 34-6 | 35-6 | 36-6 |
| Adhesion | Substrate 1 | A | A | A | A | A | A |
| | Substrate 2 | A | A | A | A | A | A |
| | Substrate 3 | A | A | A | A | A | A |
| Moist heat resistance | | A | A | A | A | A | A |
| Calixarene compound | | 37-6 | 34-4 | 35-4 | 36-4 | 37-4 | 34-7 |
| Adhesion | Substrate 1 | B | A | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 10

| Calixarene compound | | 35-7 | 36-7 | 37-7 | 34-18 | 35-18 | 36-18 |
|---|---|---|---|---|---|---|---|
| Adhesion | Substrate 1 | A | A | B | A | A | A |
| | Substrate 2 | A | A | A | A | A | A |
| | Substrate 3 | A | A | A | A | A | A |
| Moist heat resistance | | A | A | A | A | A | A |
| Calixarene compound | | 37-18 | 34-1 | 35-1 | 36-1 | 37-1 | 38-6 |
| Adhesion | Substrate 1 | B | A | A | B | B | A |
| | Substrate 2 | A | A | A | A | A | A |
| | Substrate 3 | A | A | A | A | A | A |
| Moist heat resistance | | A | A | A | A | A | A |
| Calixarene compound | | 39-6 | 40-6 | 41-6 | 42-6 | 43-6 | 44-6 |
| Adhesion | Substrate 1 | A | A | B | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 11

| Calixarene compound | | 45-6 | 46-6 | 47-6 | 48-6 | 49-6 | 50-6 |
|---|---|---|---|---|---|---|---|
| Adhesion | Substrate 1 | B | A | A | A | B | A |
| | Substrate 2 | A | A | A | A | A | A |
| | Substrate 3 | A | A | A | A | A | A |
| Moist heat resistance | | A | A | A | A | A | A |
| Calixarene compound | | 51-6 | 52-6 | 53-6 | 54-6 | 55-6 | 56-6 |
| Adhesion | Substrate 1 | A | A | B | A | A | A |
| | Substrate 2 | A | A | A | A | A | A |
| | Substrate 3 | A | A | A | A | A | A |
| Moist heat resistance | | A | A | A | A | A | A |
| Calixarene compound | | 57-6 | 58-6 | 59-6 | 60-6 | 61-6 | 62-6 |
| Adhesion | Substrate 1 | B | A | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 12

| Calixarene compound | | 63-6 | 64-6 | 65-6 | 66-6 | 66-4 | 66-7 |
|---|---|---|---|---|---|---|---|
| Adhesion | Substrate 1 | A | A | B | A | A | A |
| | Substrate 2 | A | A | A | A | A | A |
| | Substrate 3 | A | A | A | A | A | A |
| Moist heat resistance | | A | A | A | A | A | A |
| Calixarene compound | | 66-18 | 66-1 | 67-6 | 68-6 | 69-6 | 01-6 |
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 02&03-6 | 04-6 | 01-1 | 02&03-1 | 04-1 | 01-4 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 13

| Calixarene compound | | 02&034 | 04-4 | 01-7 | 02&03-7 | 04-7 | 01-18 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 02&03-18 | 04-18 | 05-6 | 05-1 | 05-4 | 05-7 |
|---|---|---|---|---|---|---|---|
| Adhesion | Substrate 1 | A | B | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 05-18 | 06-6 | 07&08-6 | 09-6 | 06-1 | 07&08-1 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 14

| Calixarene compound | | 09-1 | 06-4 | 07&084 | 09-4 | 06-7 | 07&08-7 |
|---|---|---|---|---|---|---|---|
| Adhesion | Substrate 1 | B | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 09-7 | 06-18 | 07&08-18 | 09-18 | 010-6 | 010-1 |
|---|---|---|---|---|---|---|---|
| Adhesion | Substrate 1 | B | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 010-4 | 010-7 | 010-18 |
|---|---|---|---|---|
| Adhesion | Substrate 1 | A | A | A |
| | Substrate 2 | A | A | A |
| | Substrate 3 | A | A | A |

| | | | |
|---|---|---|---|
| Moist heat resistance | A | A | A |

TABLE 15

| Calixarene compound | | 1′ | 2′ | 3′ | 4′ | 5′ | 6′ |
|---|---|---|---|---|---|---|---|
| Adhesion | Substrate 1 | B | B | B | B | C | C |
| | Substrate 2 | B | B | B | B | B | C |
| | Substrate 3 | B | B | B | B | B | C |

| | | | | | | |
|---|---|---|---|---|---|---|
| Moist heat resistance | B | B | B | B | B | B |

### Example Group <III>

### Synthesis Example 1

A 20 L separable four-necked flask equipped with a stirrer, a thermometer, and a reflux condenser tube was quickly charged with 1000 g (1.54 mol) of t-butyl-calix[4]arene, 1159 g (12.32 mol) of phenol, and 9375 mL of dehydrated toluene, and the mixture was stirred at 300 rpm under nitrogen flow. The t-butyl-calix[4]arene used as a raw material was not dissolved but was suspended. Then, while the flask was cooled in an ice bath, 1643 g (12.32 mol) of anhydrous aluminum chloride (III) was added in several portions. The solution turned into a light orange transparent solution, and the anhydrous aluminum chloride (III) precipitated on the bottom. The mixture was allowed to react at room temperature for 5 hours. Then the contents were transferred to a 1 L beaker, and 20 kg of ice, 10 L of 1N hydrochloric acid, and 20 L of chloroform were added to quench the reaction. The mixture turned into a light yellow transparent solution. This reaction mixture was transferred to a separatory funnel, and the organic layer was separated. Then the aqueous layer was extracted with 5 L of chloroform three times, and the extract was combined with the organic layer. The organic layer was pre-dried over anhydrous magnesium sulfate and filtered. The solvent was removed by evaporation using an evaporator, and a mixture of white crystals and a colorless transparent solution was thereby obtained. Methanol was added slowly to the mixture under stirring to reprecipitate the crystals. The white crystals were filtered on a Kiriyama funnel and washed with methanol. The obtained white crystals were vacuum dried (at 50° C. for 6 hours or longer) to thereby obtain 597 g of the target intermediate A. The yield was 91%.

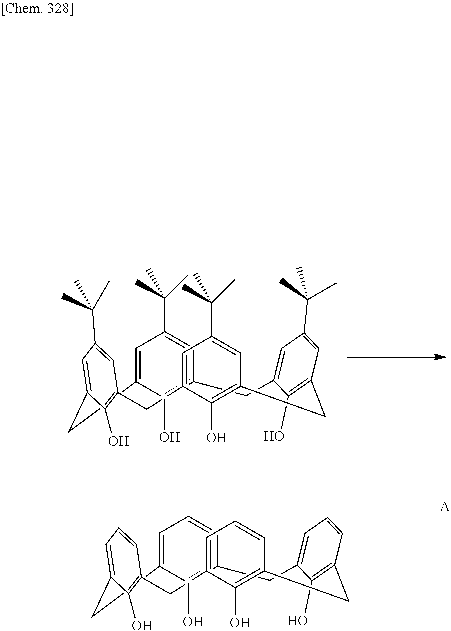

### Synthesis Example 2

A 2 L four-necked flask equipped with a stirrer, a thermometer, and a reflux condenser tube was charged with 205 g (1.52 mol) of n-hexanoyl chloride and 709 g (9.44 mol) of nitroethane, and the mixture was stirred. Then, while the flask was cooled in an ice bath, 243 g (1.82 mol) of anhydrous aluminum chloride (III) was added in several portions. The solution turned into a light orange transparent solution. The solution was stirred at room temperature for 30 minutes, and 100 g (0.236 mol) of intermediate (α-1) was added in several portions. The mixture foamed, and the reaction proceeded to thereby obtain an orange transparent solution. The resulting solution was allowed to react at room temperature for 5 hours. Then the contents were slowly transferred to a 2 L beaker containing 450 mL of chloroform and 956 g of ice water to stop the reaction. Then 1N hydrochloric acid was added until the pH was 1. The reaction mixture was transferred to a separatory funnel, and the organic layer was separated. Next, the aqueous layer was extracted with 400 mL of chloroform three times, and the extract was combined with the organic layer. The organic layer was pre-dried over anhydrous magnesium sulfate and filtered. The solvent was removed by evaporation using an evaporator, and a yellow transparent solution was thereby obtained. While the solution was cooled in an ice bath, methanol was added to reprecipitate crystals. The white crystals were filtered on a Kiriyama funnel and recrystallized with chloroform and methanol. The obtained white crystals were vacuum dried (at 60° C. for 6 hours or longer) to thereby obtain 122 g or a compound represented by the following structural formula. The yield was 63%.

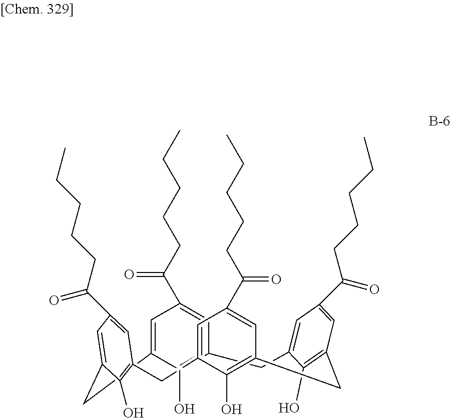 [SMILES: CCCCCC(=O)c1cc2c(O)c(c1)Cc1cc(C(=O)CCCCC)cc(c1O)Cc1cc(C(=O)CCCCC)cc(c1O)Cc1cc(C(=O)CCCCC)cc(c1O)C2]

### Synthesis Example 3

The same procedure as in Synthesis Example 2 was repeated except that butyl chloride was used instead of n-hexanoyl chloride, to thereby obtain 106 g of compound B-4 represented by the following structural formula. The yield was 64%.

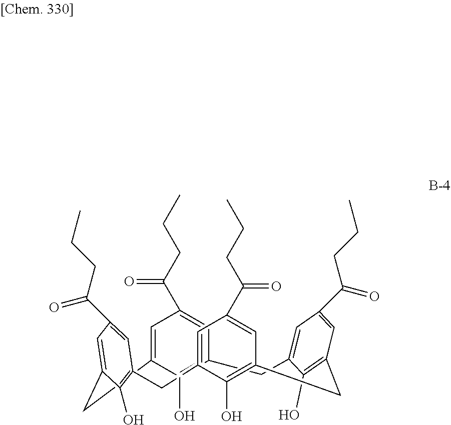 [SMILES: CCCC(=O)c1cc2c(O)c(c1)Cc1cc(C(=O)CCC)cc(c1O)Cc1cc(C(=O)CCC)cc(c1O)Cc1cc(C(=O)CCC)cc(c1O)C2]

### Synthesis Example 4

The same procedure as in Synthesis Example 2 was repeated except that n-heptanoyl chloride was used instead of n-hexanoyl chloride, to thereby obtain 134 g of compound B-7 represented by the following structural formula. The yield was 65%.

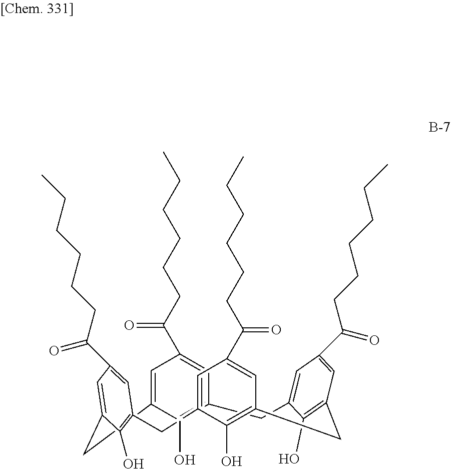 [SMILES: CCCCCCC(=O)c1cc2c(O)c(c1)Cc1cc(C(=O)CCCCCC)cc(c1O)Cc1cc(C(=O)CCCCCC)cc(c1O)Cc1cc(C(=O)CCCCCC)cc(c1O)C2]

### Synthesis Example 5

The same procedure as in Synthesis Example 2 was repeated except that stearoyl chloride was used instead of n-hexanoyl chloride, to thereby obtain 228 g of compound B-18 represented by the following structural formula. The yield was 65%.

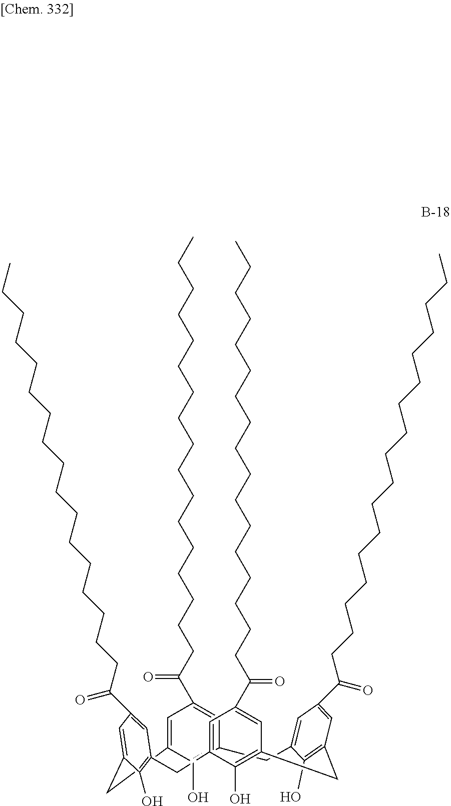 [SMILES: CCCCCCCCCCCCCCCCCC(=O)c1cc2c(O)c(c1)Cc1cc(C(=O)CCCCCCCCCCCCCCCCC)cc(c1O)Cc1cc(C(=O)CCCCCCCCCCCCCCCCC)cc(c1O)Cc1cc(C(=O)CCCCCCCCCCCCCCCCC)cc(c1O)C2]

### Synthesis Example 6

A 100 mL four-necked flask equipped with a stirrer, a thermometer, and a reflux condenser tube was charged with 5.00 g (6.119 mmol) of B-6, 17.0 g of anhydrous acetonitrile, 11.28 g (48.95 mmol) of potassium carbonate, 0.813 g (4.896 mmol) of potassium iodide, and 7.489 g (48.95 mmol) of methyl 2-bromoacetate, and the mixture was stirred at 70° C. for 24 hours. The mixture was cooled to room temperature, and ion exchanged water and 0.3N hydrochloric acid were added until the pH was 6. Then 50 g of chloroform was added. The reaction mixture was transferred to a separatory funnel, and the organic layer was separated. Next, the aqueous layer was extracted with 50 g of chloroform three times, and the extract was combined with the organic layer. The organic layer was pre-dried over anhydrous magnesium sulfate and filtered. The solvent was removed by evaporation using an evaporator to thereby obtain a red waxy solid. The obtained red waxy solid was vacuum dried (at 60° C. for 6 hours or longer) to thereby obtain 5.04 g of compound C-6 represented by the following structural formula. The yield was 74.5%.

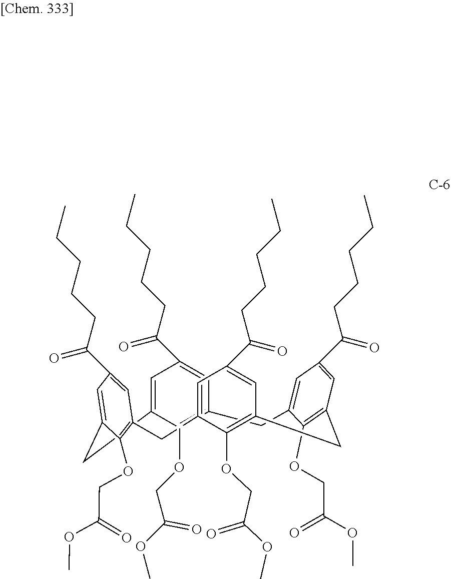 [SMILES: CCCCCC(=O)c1cc2c(OCC(=O)OC)c(c1)Cc1cc(C(=O)CCCCC)cc(c1OCC(=O)OC)Cc1cc(C(=O)CCCCC)cc(c1OCC(=O)OC)Cc1cc(C(=O)CCCCC)cc(c1OCC(=O)OC)C2]

### Synthesis Example 7

The same procedure as in Synthesis Example 6 was repeated except that B-4 was used instead of B-6, to thereby obtain 4.88 g of the target compound C-4 with a yield of 69.3%.

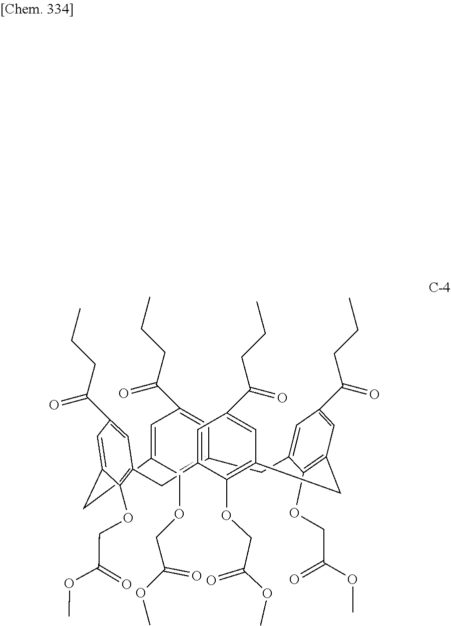 [SMILES: CCCC(=O)c1cc2c(OCC(=O)OC)c(c1)Cc1cc(C(=O)CCC)cc(c1OCC(=O)OC)Cc1cc(C(=O)CCC)cc(c1OCC(=O)OC)Cc1cc(C(=O)CCC)cc(c1OCC(=O)OC)C2]

### Synthesis Example 8

The same procedure as in Synthesis Example 6 was repeated except that B-7 was used instead of B-6, to thereby obtain 5.12 g of the target compound C-7 with a yield of 77.0%.

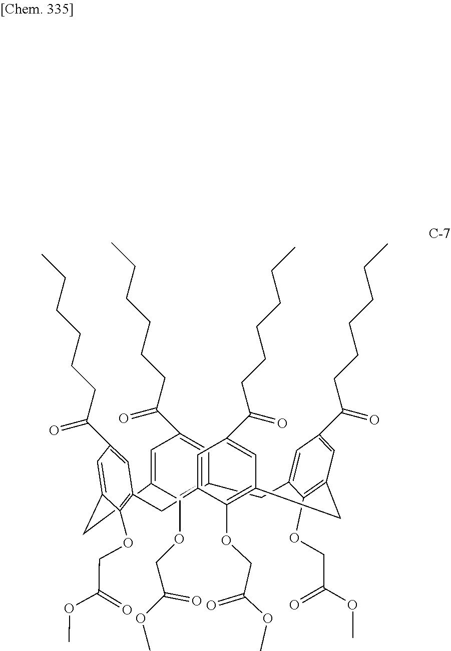 [SMILES: CCCCCCC(=O)c1cc2c(OCC(=O)OC)c(c1)Cc1cc(C(=O)CCCCCC)cc(c1OCC(=O)OC)Cc1cc(C(=O)CCCCCC)cc(c1OCC(=O)OC)Cc1cc(C(=O)CCCCCC)cc(c1OCC(=O)OC)C2]

### Synthesis Example 9

The same procedure as in Synthesis Example 6 was repeated except that B-18 was used instead of B-6, to thereby obtain 5.34 g of the target compound C-18 with a yield of 89.5%.

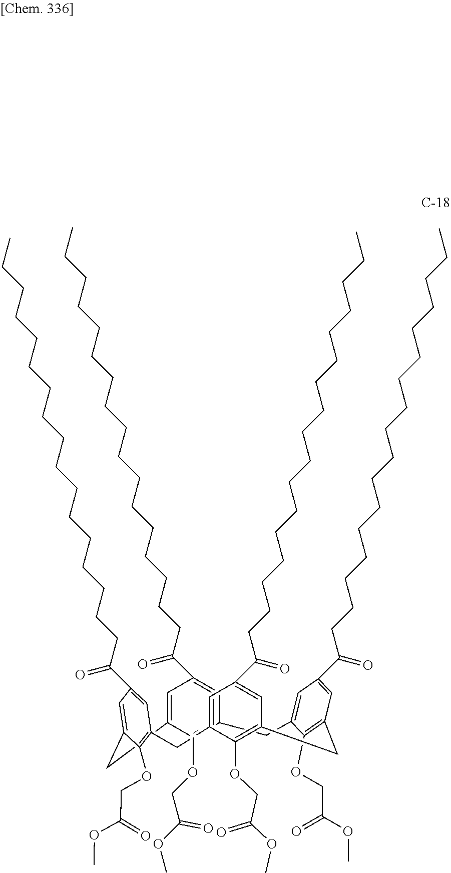 [SMILES: CCCCCCCCCCCCCCCCCC(=O)c1cc2c(OCC(=O)OC)c(c1)Cc1cc(C(=O)CCCCCCCCCCCCCCCCC)cc(c1OCC(=O)OC)Cc1cc(C(=O)CCCCCCCCCCCCCCCCC)cc(c1OCC(=O)OC)Cc1cc(C(=O)CCCCCCCCCCCCCCCCC)cc(c1OCC(=O)OC)C2]

### Synthesis Example 10

A 500 mL four-necked flask equipped with a stirrer, a thermometer, and a reflux condenser tube was charged with 16.44 g of dehydrated tetrahydrofuran in an ice bath, and 1.038 g (27.35 mmol) of lithium aluminum hydride was slowly added. 5.04 g (4.559 mmol) of C-6 diluted with 49.31 g of dehydrated tetrahydrofuran was added from a dropping funnel such that the temperature did not exceed 10° C. The resulting gray suspension reaction solution was allowed to react at room temperature for 6 hours. 30 g of chloroform was added to the resulting solution placed in an ice bath, and 30 g of 5N hydrochloric acid was added dropwise to stop the reaction. Then diatomaceous earth was used to filter the reaction solution, and the filtrate was transferred to a separatory funnel to separate the organic layer. Next, the aqueous layer was extracted with 30 g of chloroform three times, and the extract was combined with the organic layer. The organic layer was pre-dried over anhydrous magnesium sulfate, and the solvent was removed by evaporation using an evaporator. The obtained light yellow liquid was subjected to column chromatography (eluent: n-hexane:ethyl acetate=1:1) to remove by-products and purified by column chromatography using chloroform:isopropyl alcohol=5:1) to thereby obtain 2.857 g of the target compound D-6 as white crystals. The yield was 63.1%.

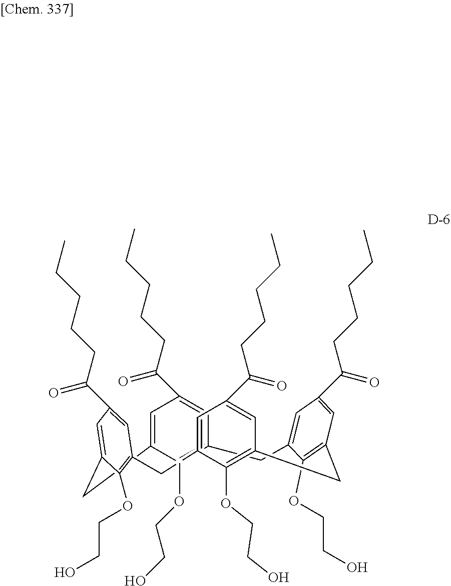 [SMILES: CCCCCC(=O)c1cc2c(OCCO)c(c1)Cc1cc(C(=O)CCCCC)cc(c1OCCO)Cc1cc(C(=O)CCCCC)cc(c1OCCO)Cc1cc(C(=O)CCCCC)cc(c1OCCO)C2]

### Synthesis Example 11

The same procedure as in Synthesis Example 10 was repeated except that C-4 was used instead of C-6, to thereby obtain 3.06 g of the target compound D-4 with a yield of 69.0%.

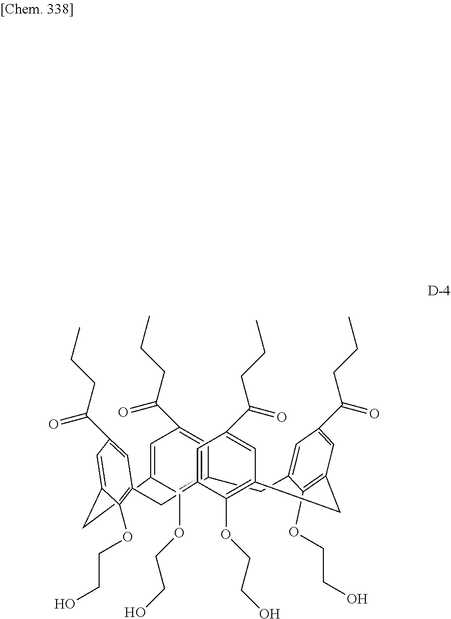 [SMILES: CCCC(=O)c1cc2c(OCCO)c(c1)Cc1cc(C(=O)CCC)cc(c1OCCO)Cc1cc(C(=O)CCC)cc(c1OCCO)Cc1cc(C(=O)CCC)cc(c1OCCO)C2]

### Synthesis Example 12

The same procedure as in Synthesis Example 10 was repeated except that C-7 was used instead of C-6, to thereby obtain 3.11 g of the target compound D-7 with a yield of 68.2%.

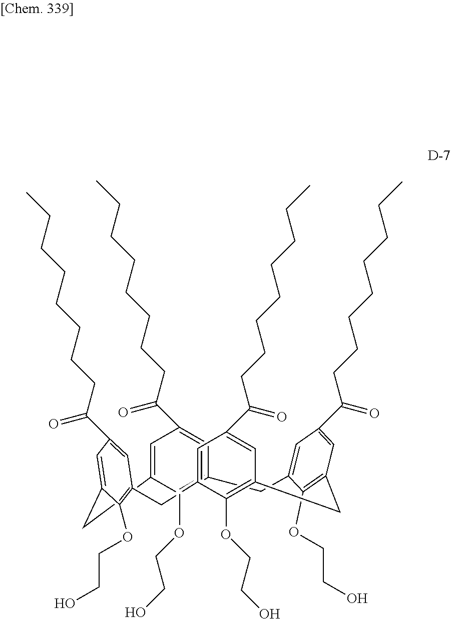 [SMILES: CCCCCCCCC(=O)c1cc2c(OCCO)c(c1)Cc1cc(C(=O)CCCCCCCC)cc(c1OCCO)Cc1cc(C(=O)CCCCCCCC)cc(c1OCCO)Cc1cc(C(=O)CCCCCCCC)cc(c1OCCO)C2]

### Example 1

A 50 mL four-necked flask equipped with a stirrer, a thermometer, and a reflux condenser tube was charged with 1.00 g (1.007 mmol) of D-6, 3.63 g of tetrahydrofuran, 2.112 g (8.054 mmol) of triphenyiphosphine, 0.173 g (2.014 mmol) of methacrylic acid, and 0.617 g (6.041 mmol) of 2-acetylacetic acid, and the mixture was stirred. Then 1.810 g (8.054 mmol) of diisopropyl azodicarboxylate diluted with 1.742 g of tetrahydrofuran was added dropwise to the mixture placed in an ice bath over 30 minutes. The resulting orange transparent reaction solution was stirred at room temperature for 10 hours. Hexane was added to the reaction solution to precipitate and remove by-products such as triphenylphosphine, then the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:ethyl acetate=85:15), to thereby obtain 0.359 g of the target compound 1-6 with a yield of 27.1%, 0.201 g of the target compound 2-6 with a yield of 15.4%, 0.197 g of the target compound 3-6 with a yield of 15.1%, and 0.106 g of the target compound 4-6 with a yield of 8.2%.

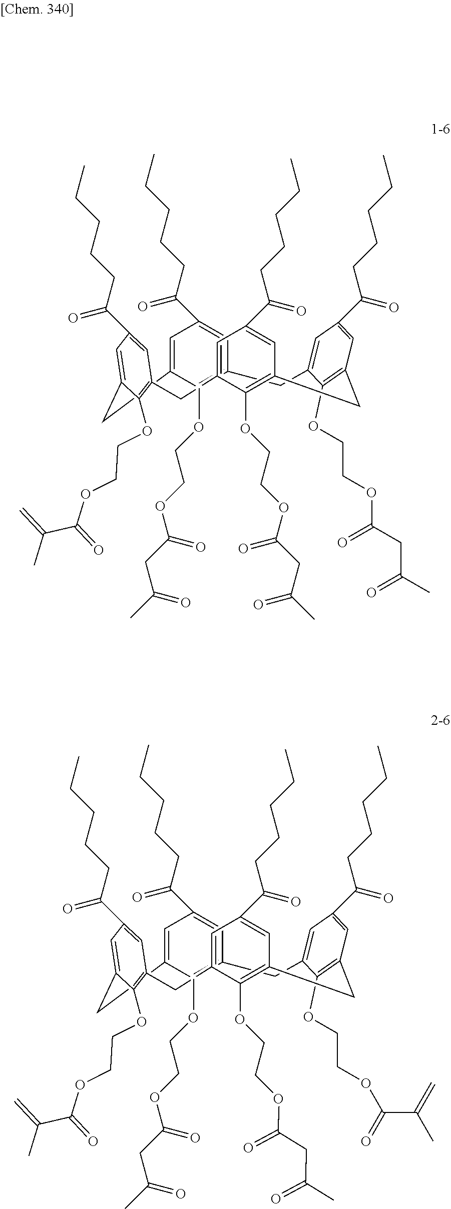

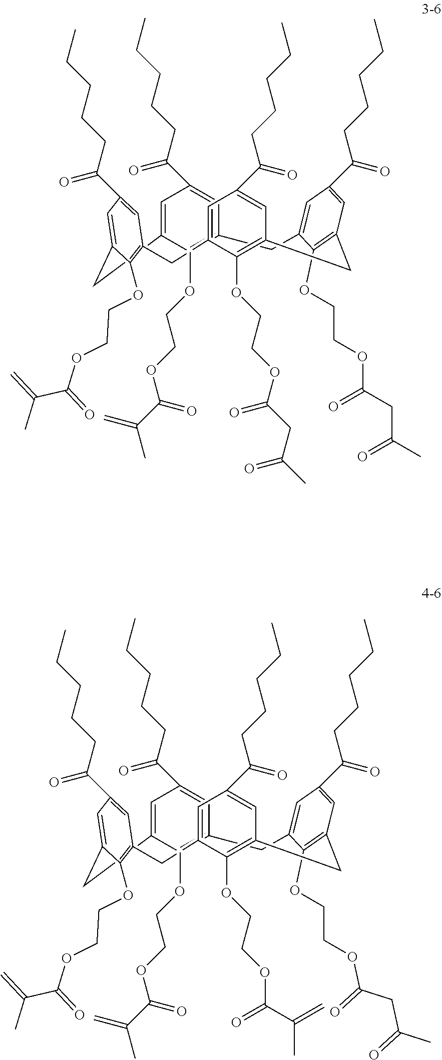

### Example 2

The same procedure as in Example 1 was repeated except that D-4 was used instead of D-6, to thereby obtain 0.334 g of the target compound 1-4 with a yield of 24.5%, 0.187 g of the target compound 2-4 with a yield of 13.9%, 0.175 g of the target compound 3-4 with a yield of 13.0%, and 0.108 g of the target compound 4-4 with a yield of 8.14%.

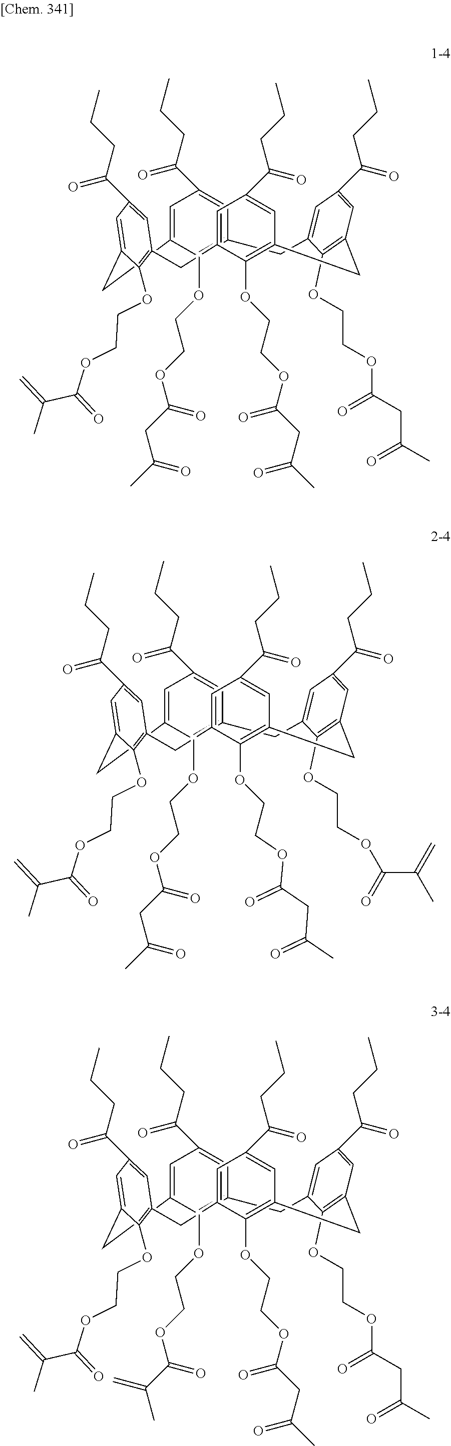

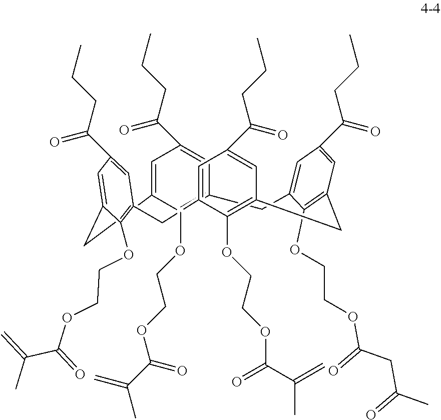 [SMILES: C=C(C)C(=O)OCCOc1c2cc(C(=O)CCC)cc1Cc1cc(C(=O)CCC)cc(c1OCCOC(=O)C(=C)C)Cc1cc(C(=O)CCC)cc(c1OCCOC(=O)C(=C)C)Cc1cc(C(=O)CCC)cc(c1OCCOC(=O)CC(C)=O)C2]

### Example 3

The same procedure as in Example 1 was repeated except that D-7 was used instead of D-6, to thereby obtain 0.345 g of the target compound 1-7 with a yield of 26.4%, 0.194 g of the target compound 2-7 with a yield of 15.0%, 0.186 g of the target compound 3-7 with a yield of 14.4%, and 0.111 g of the target compound 4-7 with a yield of 8.71%.

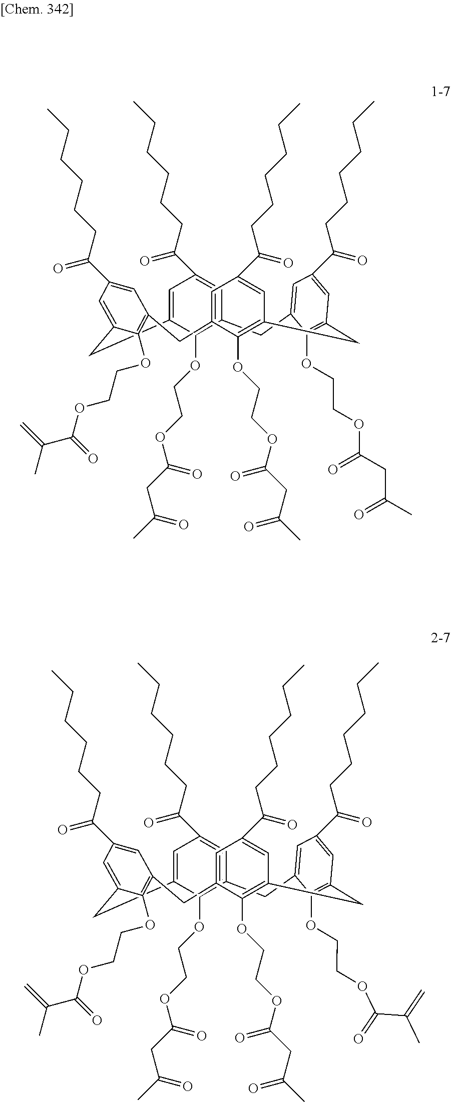

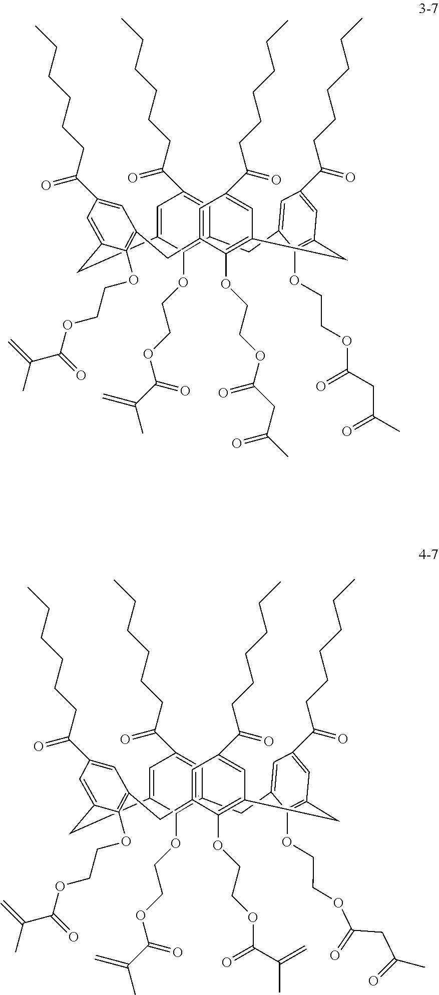

### Example 4

The same procedure as in Example 1 was repeated except that acrylic acid was used instead of methacrylic acid, to thereby obtain 0.351 g of the target compound 5-6 with a yield of 26.8%, 0.217 g of the target compound 6-6 with a yield of 17.0%, 0.209 g of the target compound 7-6 with a yield of 16.4%, and 0.131 g of the target compound 8-6 with a yield of 10.5%.

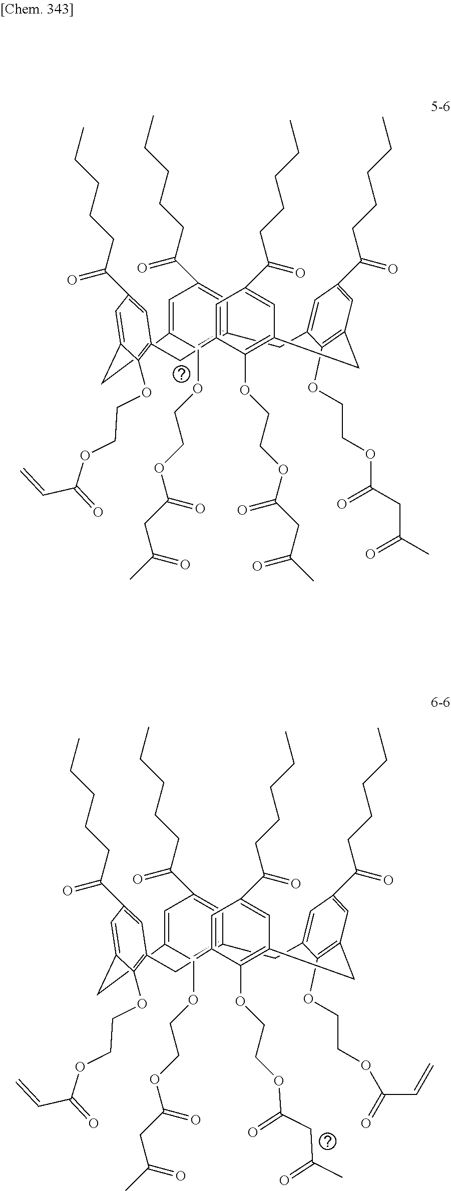

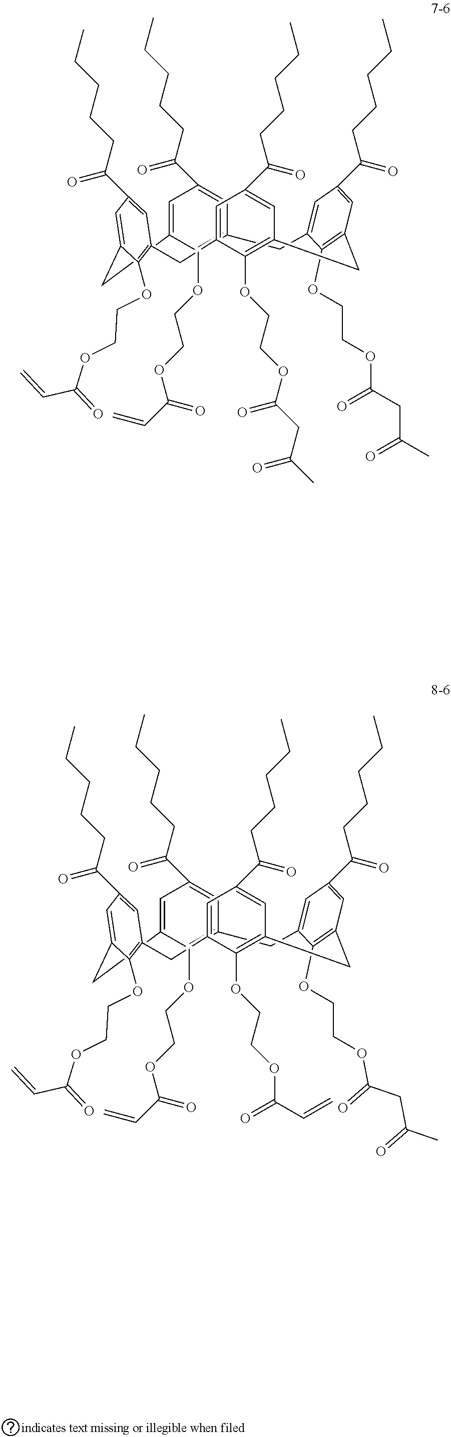

### Example 5

The same procedure as in Example 1 was repeated except that 3-oxopentanoic acid was used instead of 2-acetylacetic acid, to thereby obtain 0.361 g of the target compound 9-6 with a yield of 26.4%, 0.226 g of the target compound 10-6 with a yield of 16.9%, 0.218 g of the target compound 11-6 with a yield of 16.3%, and 0.135 g of the target compound 12-6 with a yield of 10.3%.

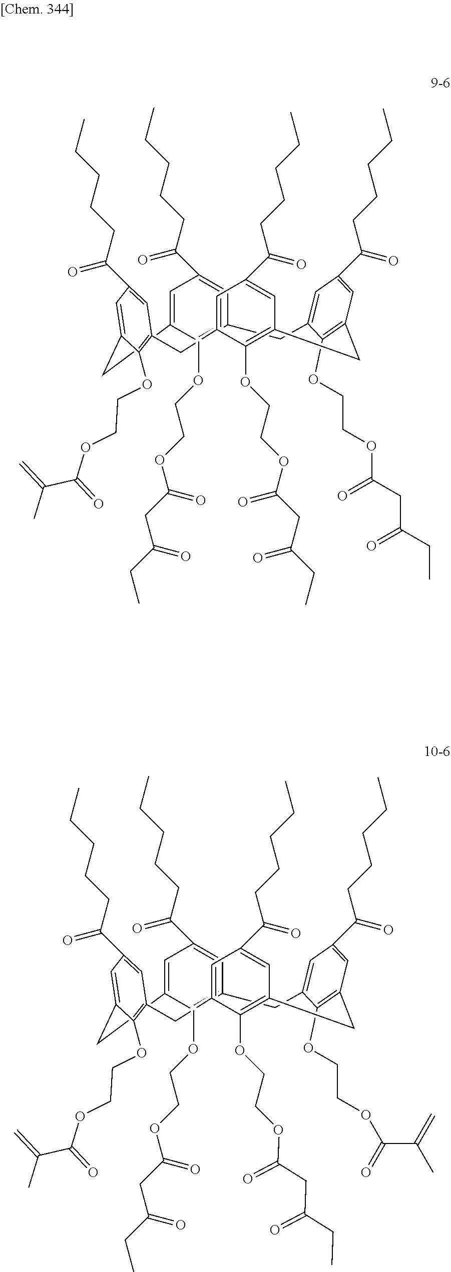

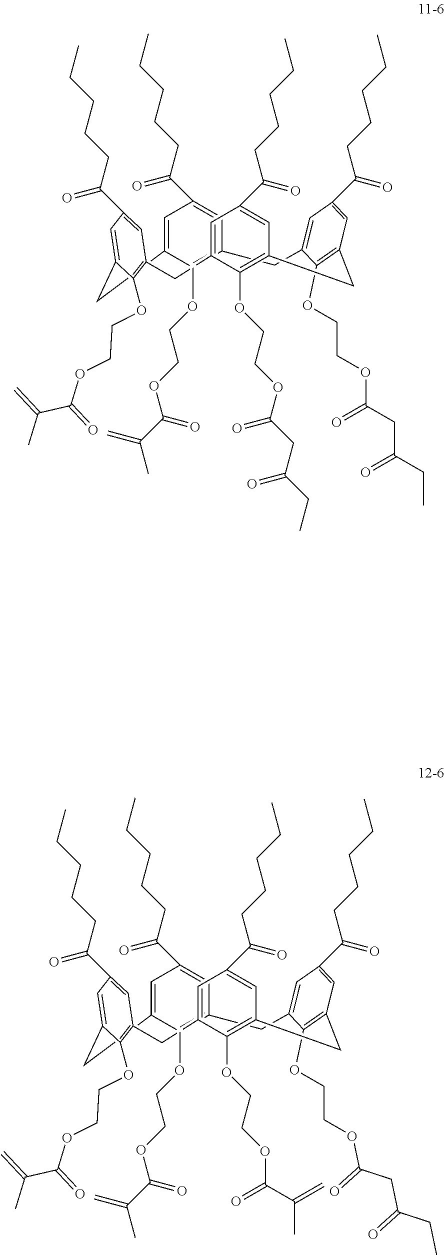

### Example 6

The same procedure as in Example 5 was repeated except that D-4 was used instead of D-6, to thereby obtain 0.331 g of the target compound 9-4 with a yield of 23.7%, 0.209 g of the target compound 10-4 with a yield of 15.3%, 0.197 g of the target compound 11-4 with a yield of 14.5%, and 0.102 g of the target compound 12-4 with a yield of 7.68%.

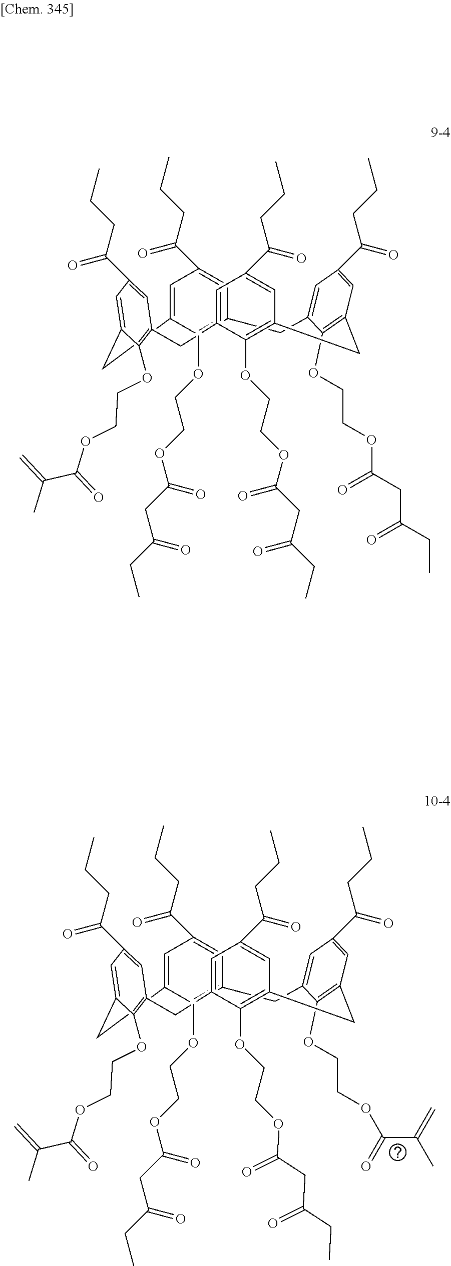

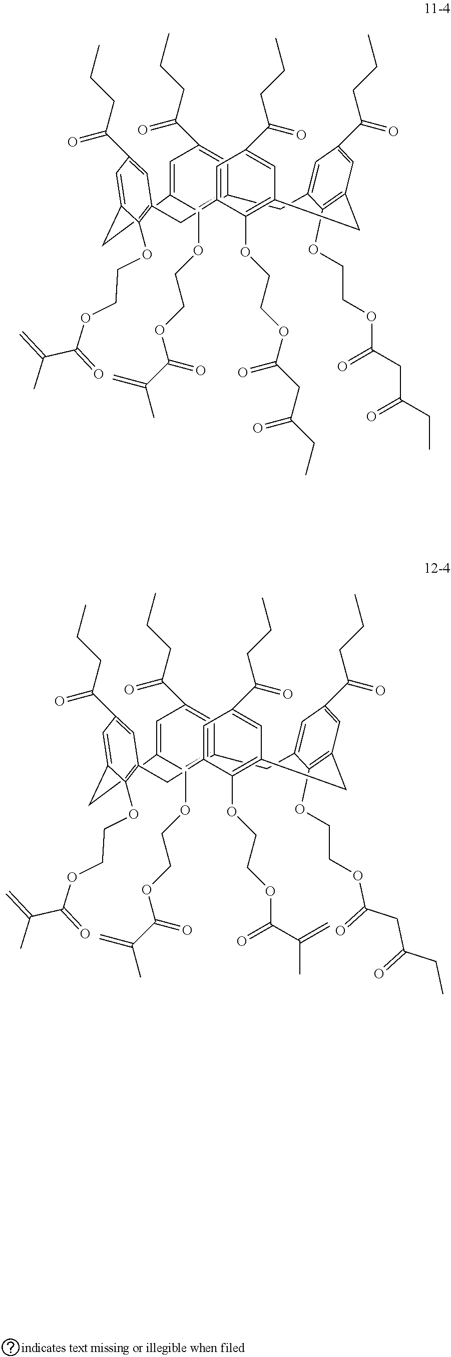

### Example 7

The same procedure as in Example 5 was repeated except that D-7 was used instead of D-6, to thereby obtain 0.345 g of the target compound 9-7 with a yield of 25.6%, 0.221 g of the target compound 10-7 with a yield of 16.8%, 0.228 g of the target compound 11-7 with a yield of 17.3%, and 0.130 g of the target compound 12-7 with a yield of 10.1%.

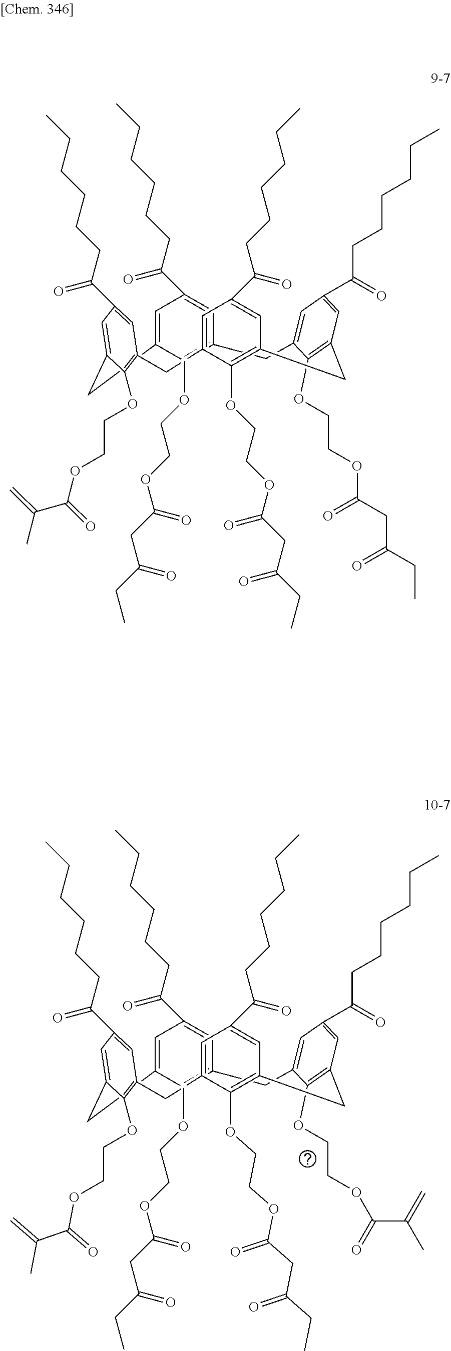

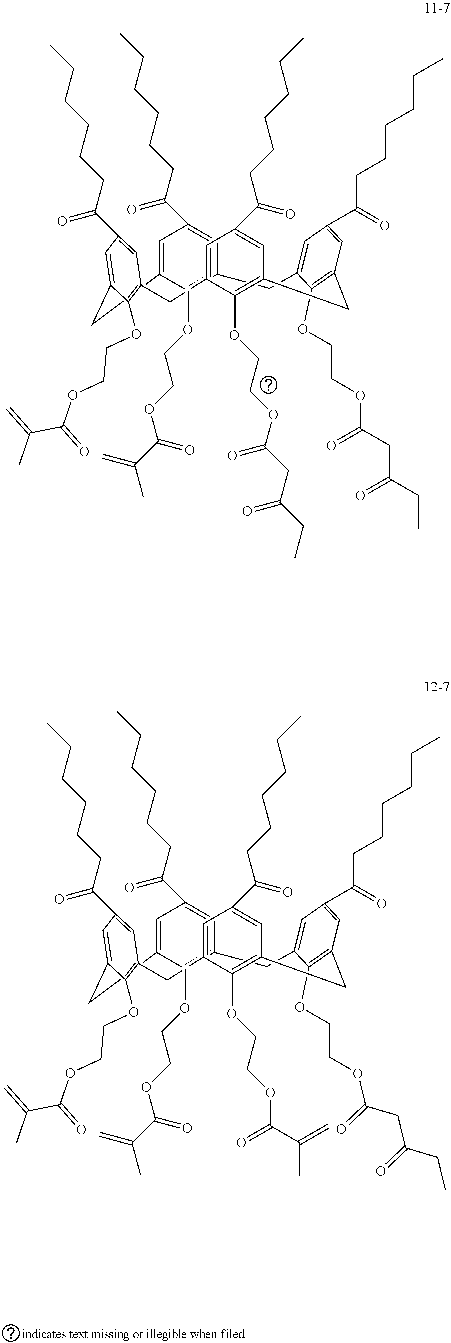

### Example 8

The same procedure as in Example 5 was repeated except that acrylic acid was used instead of methacrylic acid, to thereby obtain 0.329 g of the target compound 13-6 with a yield of 24.4%, 0.216 g of the target compound 14-6 with a yield of 16.5%, 0.217 g of the target compound 15-6 with a yield of 16.6%, and 0.125 g of the target compound 16-6 with a yield of 9.90%.

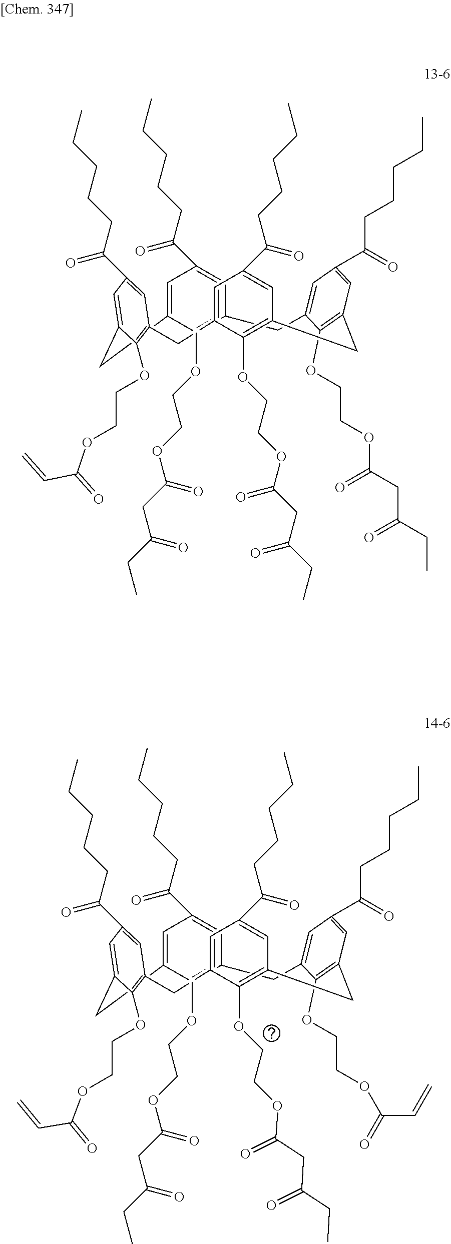

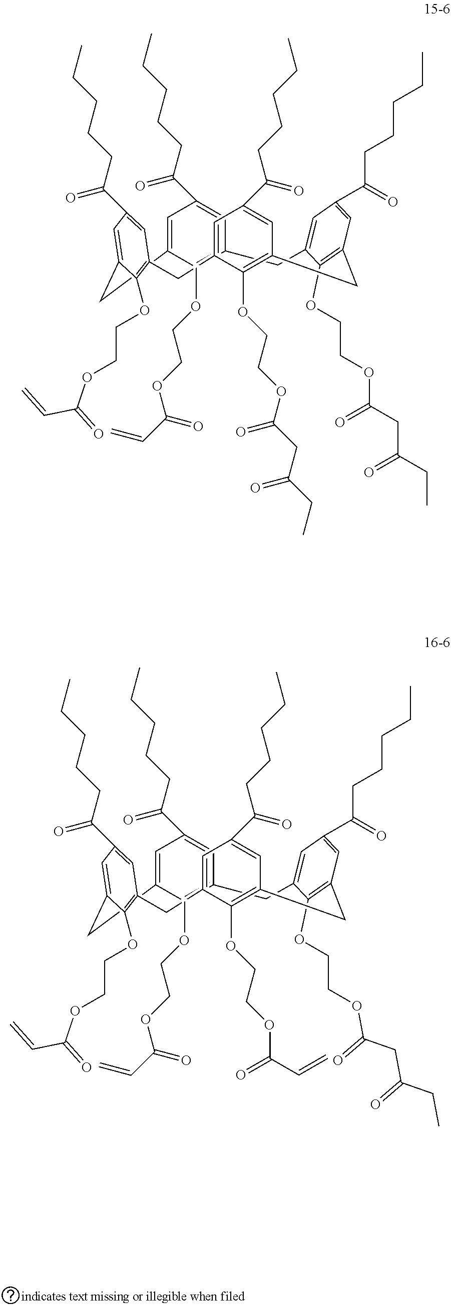

### Synthesis Example 13

A 500 mL four-necked flask equipped with a stirrer, a thermometer, and a reflux condenser tube was charged with 92.6 g (113.33 mmol) of B-6 and 944.52 g of diethylene glycol monomethyl ether, and the mixture was stirred. Then 46.4 mL (906.64 mmol) of hydrazine monohydrate and 50.9 g (906.64 mmol) of potassium hydroxide were added, and the mixture was stirred at 100° C. for 30 minutes and then heat-refluxed for 8 hours. After completion of the reaction, the resulting mixture was cooled to 90° C. 92.6 mL of ion exchanged water was added, and the mixture was stirred for 30 minutes. Then the mixture was cooled to room temperature, and 6N hydrochloric acid was added until the pH was 1. 300 g of chloroform was added, and the organic layer was separated. Next, the aqueous layer was extracted with 300 g of chloroform three times, and the extract was combined with the organic layer. The organic layer was pre-dried over anhydrous magnesium sulfate, and the solvent was removed by evaporation using an evaporator to thereby obtain an orange viscus liquid. Methanol was added to reprecipitate crystals, and the white crystals were filtered on a Kiriyama funnel. The obtained milky white crystals were vacuum dried (at 60° C. for 6 hours or longer) to thereby obtain 54.34 g of the target compound E-6. The yield was 63.0%.

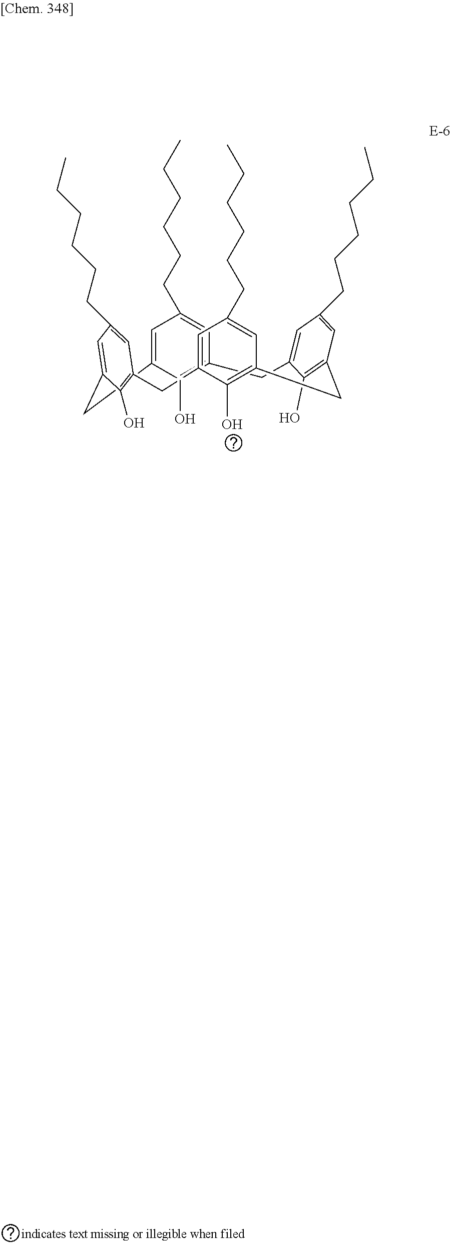 [SMILES: CCCCCCc1cc2c(O)c(c1)Cc1cc(CCCCCC)cc(c1O)Cc1cc(CCCCCC)cc(c1O)Cc1cc(CCCCCC)cc(c1O)C2]

### Synthesis Example 14

The same procedure as in Synthesis Example 13 was repeated except that B-4 was used instead of B-6, to thereby obtain 72.45 g of the target compound E-4. The yield was 83.1%.

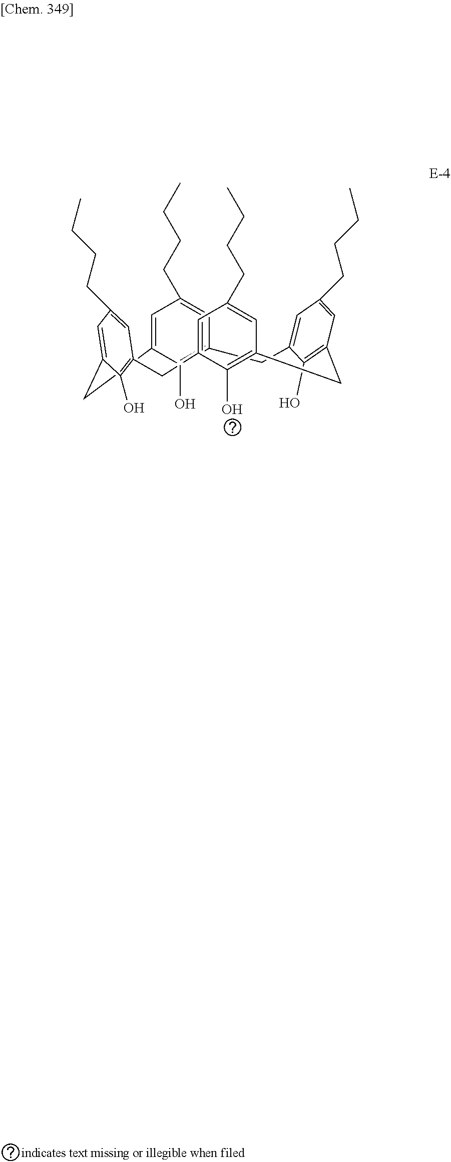 [SMILES: CCCCc1cc2c(O)c(c1)Cc1cc(CCCC)cc(c1O)Cc1cc(CCCC)cc(c1O)Cc1cc(CCCC)cc(c1O)C2]

### Synthesis Example 15

The same procedure as in Synthesis Example 13 was repeated except that B-7 was used instead of B-6, to thereby obtain 78.4 g of the target compound E-7. The yield was

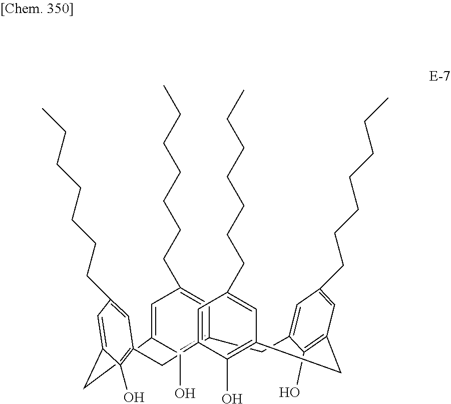 [SMILES: CCCCCCCc1cc2c(O)c(c1)Cc1cc(CCCCCCC)cc(c1O)Cc1cc(CCCCCCC)cc(c1O)Cc1cc(CCCCCCC)cc(c1O)C2]

### Synthesis Example 16

The same procedure as in Synthesis Example 13 was repeated except that B-18 was used instead of B-6, to thereby obtain 37.9 g of the target compound E-18. The yield was 96.0%.

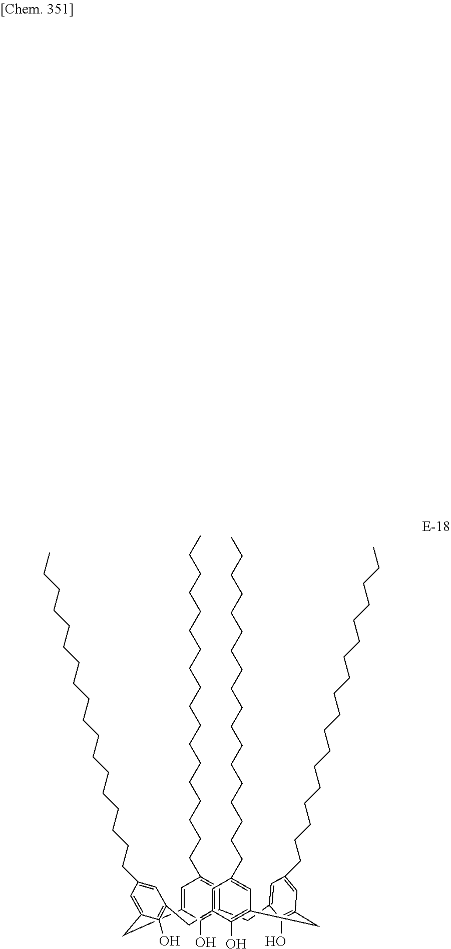 [SMILES: CCCCCCCCCCCCCCCCCCc1cc2c(O)c(c1)Cc1cc(CCCCCCCCCCCCCCCCCC)cc(c1O)Cc1cc(CCCCCCCCCCCCCCCCCC)cc(c1O)Cc1cc(CCCCCCCCCCCCCCCCCC)cc(c1O)C2]

### Synthesis Example 17

Referring to known documents (Tetrahedron Letters, 43(43), 7691-7693; 2002, and Tetrahedron Letters, 48(5), 905-12; 1992), compound E-1 was synthesized according to the following scheme (yield amount 75 g, yield 66.6%).

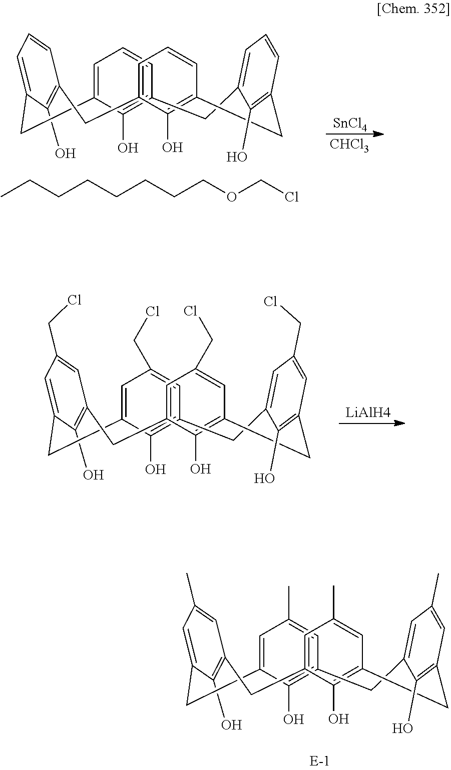

### Synthesis Example 18

A 1 L four-necked flask equipped with a stirrer, a thermometer, and a reflux condenser tube was charged with 20.00 g (26.276 mmol) of E-6, 400 g of anhydrous acetonitrile, 15.29 g (105.11 mmol) of potassium carbonate, 10.511 g (10.511 mmol) of potassium iodide, and 32.158 g (210.21 mmol) of methyl 2-bromoacetate, and the mixture was heated to 70° C. for 6 hours. The resulting mixture was cooled to room temperature, and ion exchanged water and 1N hydrochloric acid were added until the pH was 6. Then 500 g of chloroform was added. The reaction mixture was transferred to a separatory funnel, and the organic layer was separated. Next, the aqueous layer was extracted with 100 g of chloroform three times, and the extract was combined with the organic layer. The organic layer was pre-dried over anhydrous magnesium sulfate and filtered. The solvent was removed by evaporation using an evaporator to thereby obtain a red waxy solid. The obtained red waxy solid was vacuum dried (at 60° C. for 6 hours or longer) to thereby obtain 21.67 g of the target compound F-6. The yield was 78.6%.

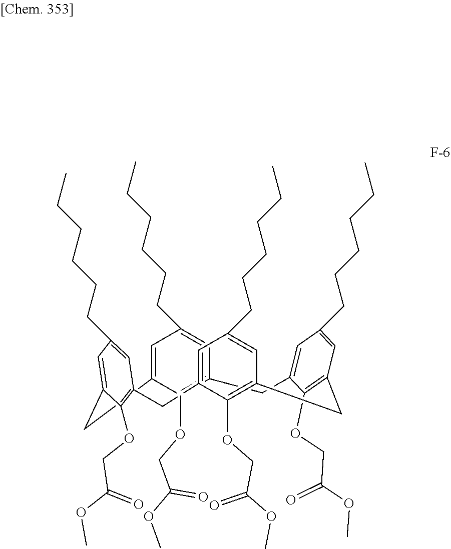 [SMILES: CCCCCCc1cc2c(OCC(=O)OC)c(c1)Cc1cc(CCCCCC)cc(c1OCC(=O)OC)Cc1cc(CCCCCC)cc(c1OCC(=O)OC)Cc1cc(CCCCCC)cc(c1OCC(=O)OC)C2]

### Synthesis Example 19

The same procedure as in Synthesis Example 18 was repeated except that E-4 was used instead of E-6, to thereby obtain 21.81 g of the target compound F-4. The yield was 75.5%.

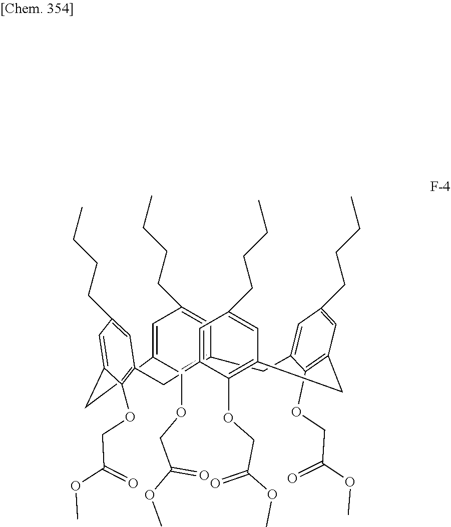 [SMILES: CCCCc1cc2c(OCC(=O)OC)c(c1)Cc1cc(CCCC)cc(c1OCC(=O)OC)Cc1cc(CCCC)cc(c1OCC(=O)OC)Cc1cc(CCCC)cc(c1OCC(=O)OC)C2]

### Synthesis Example 20

The same procedure as in Synthesis Example 18 was repeated except that E-7 was used instead of E-6, to thereby obtain 20.98 g of the target compound F-7. The yield was 77.5%.

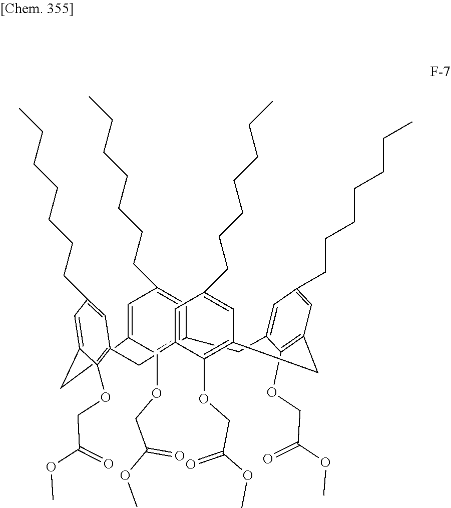 [SMILES: CCCCCCCc1cc2c(OCC(=O)OC)c(c1)Cc1cc(CCCCCCC)cc(c1OCC(=O)OC)Cc1cc(CCCCCCC)cc(c1OCC(=O)OC)Cc1cc(CCCCCCC)cc(c1OCC(=O)OC)C2]

### Synthesis Example 21

The same procedure as in Synthesis Example 18 was repeated except that E-18 was used instead of E-6, to thereby obtain 19.32 g of the target compound F-18. The yield was 80.4%.

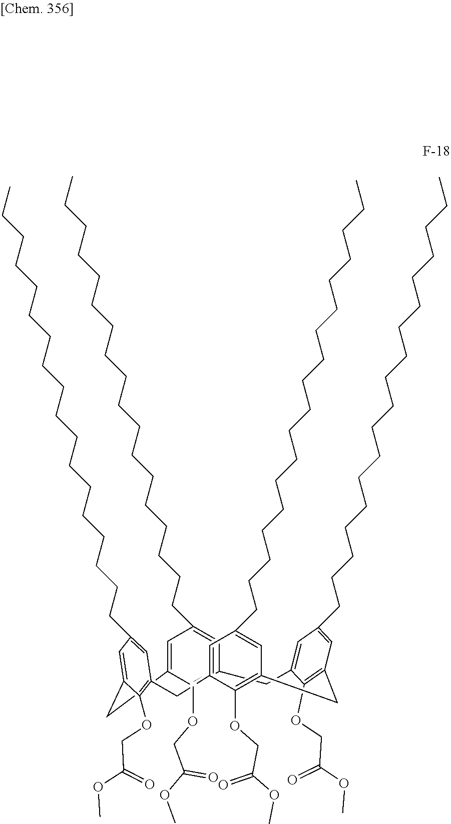 [SMILES: CCCCCCCCCCCCCCCCCCc1cc2c(OCC(=O)OC)c(c1)Cc1cc(CCCCCCCCCCCCCCCCCC)cc(c1OCC(=O)OC)Cc1cc(CCCCCCCCCCCCCCCCCC)cc(c1OCC(=O)OC)Cc1cc(CCCCCCCCCCCCCCCCCC)cc(c1OCC(=O)OC)C2]

### Synthesis Example 22

The same procedure as in Synthesis Example 18 was repeated except that E-1 was used instead of E-6, to thereby obtain 18.32 g of the target compound F-1. The yield was

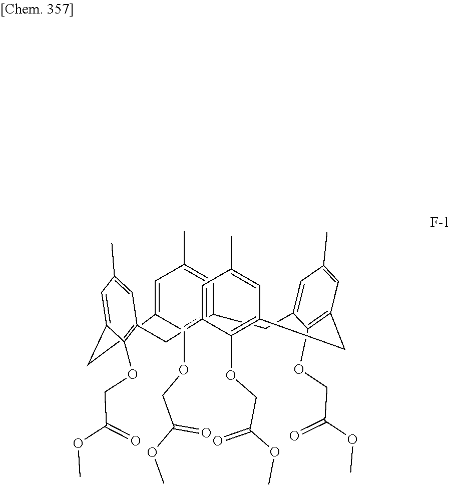 [SMILES: COC(=O)COc1c2cc(C)cc1Cc1cc(C)cc(c1OCC(=O)OC)Cc1cc(C)cc(c1OCC(=O)OC)Cc1cc(C)cc(c1OCC(=O)OC)C2]

### Synthesis Example 23

The same procedure as in Synthesis Example 10 was repeated except that F-6 was used instead of C-6, to thereby obtain 6.12 g of the target compound G-6. The yield was 68.5%.

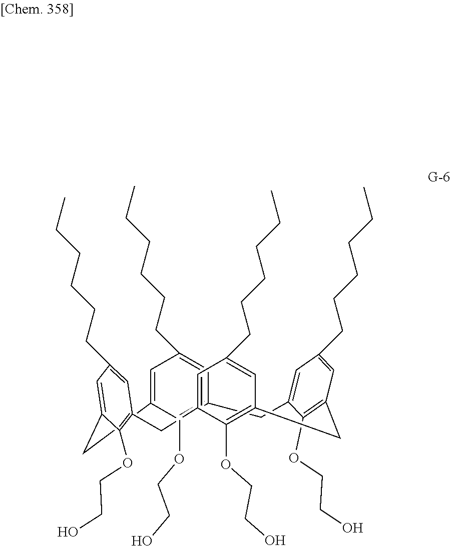 [SMILES: CCCCCCc1cc2c(OCCO)c(c1)Cc1cc(CCCCCC)cc(c1OCCO)Cc1cc(CCCCCC)cc(c1OCCO)Cc1cc(CCCCCC)cc(c1OCCO)C2]

### Synthesis Example 24

The same procedure as in Synthesis Example 10 was repeated except that F-4 was used instead of C-6, to thereby obtain 4.21 g of the target compound G-4. The yield was 81.4%.

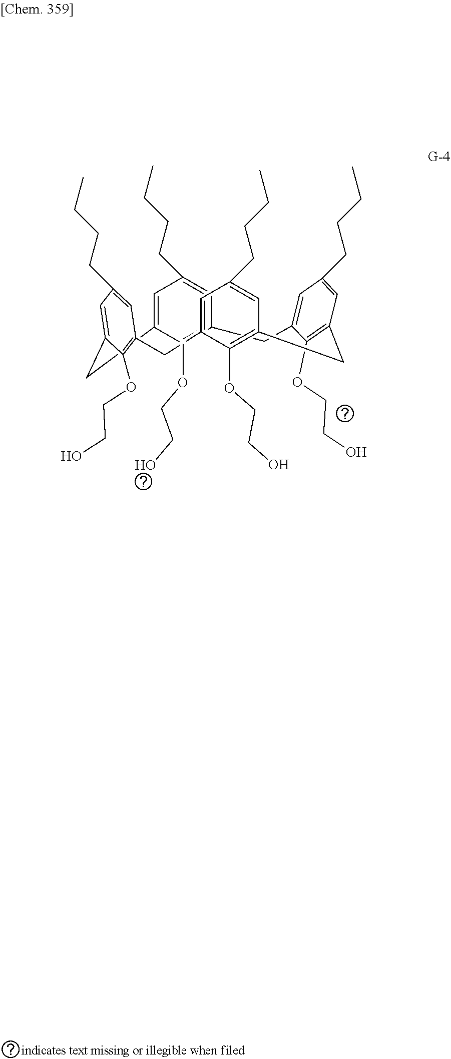 [SMILES: CCCCc1cc2c(OCCO)c(c1)Cc1cc(CCCC)cc(c1OCCO)Cc1cc(CCCC)cc(c1OCCO)Cc1cc(CCCC)cc(c1OCCO)C2]

### Synthesis Example 25

The same procedure as in Synthesis Example 10 was repeated except that F-7 was used instead of C-6, to thereby obtain 3.89 g of the target compound G-7. The yield was 84.5%.

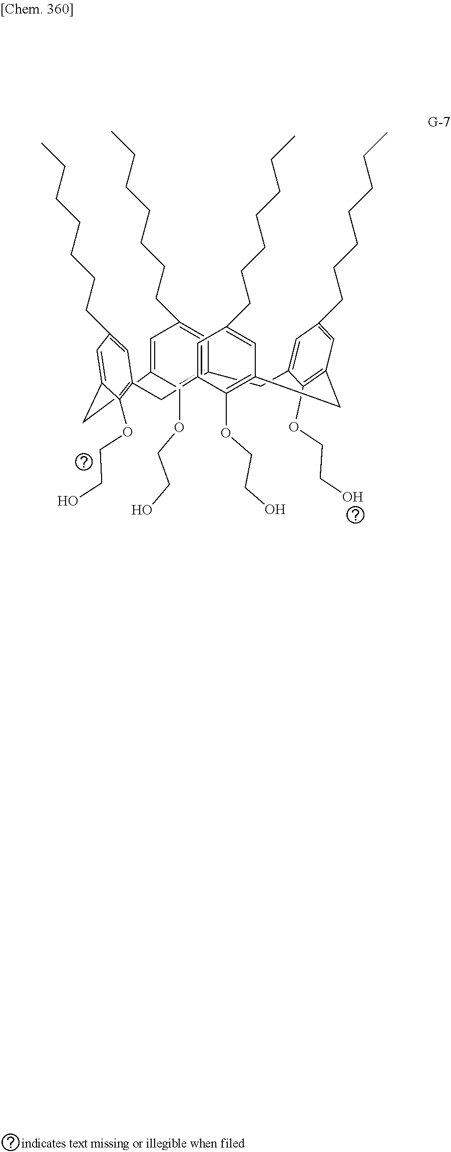 [SMILES: CCCCCCCc1cc2c(OCCO)c(c1)Cc1cc(CCCCCCC)cc(c1OCCO)Cc1cc(CCCCCCC)cc(c1OCCO)Cc1cc(CCCCCCC)cc(c1OCCO)C2]

### Synthesis Example 26

The same procedure as in Synthesis Example 10 was repeated except that F-18 was used instead of C-6, to thereby obtain 4.31 g of the target compound G-18. The yield was 81.7%.

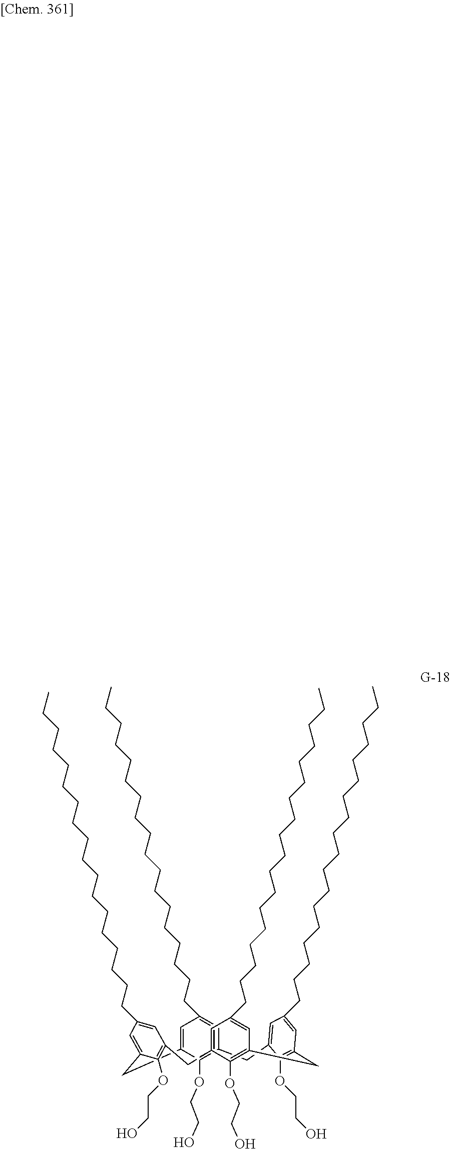 [SMILES: CCCCCCCCCCCCCCCCCCc1cc2c(OCCO)c(c1)Cc1cc(CCCCCCCCCCCCCCCCCC)cc(c1OCCO)Cc1cc(CCCCCCCCCCCCCCCCCC)cc(c1OCCO)Cc1cc(CCCCCCCCCCCCCCCCCC)cc(c1OCCO)C2]

### Synthesis Example 27

The same procedure as in Synthesis Example 10 was repeated except that H-1 was used instead of C-6, to thereby obtain 3.43 g of the target compound G-1. The yield was 85.1%.

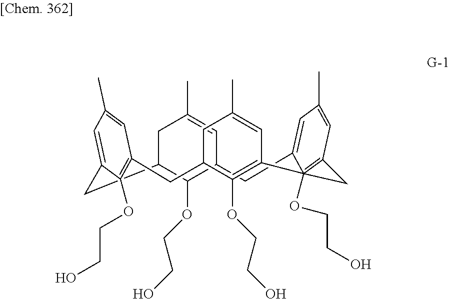

### Example 9

The same procedure as in Example 1 was repeated except that G-6 was used instead of D-6, to thereby obtain 0.412 g of the target compound 17-6. The yield was 30.7%. 0.201 g of 18-6 was obtained. The yield was 15.2%. 0.217 g of 19-6 was obtained. The yield was 16.4%. 0.137 g of 20-6 was obtained. The yield was 10.5%.

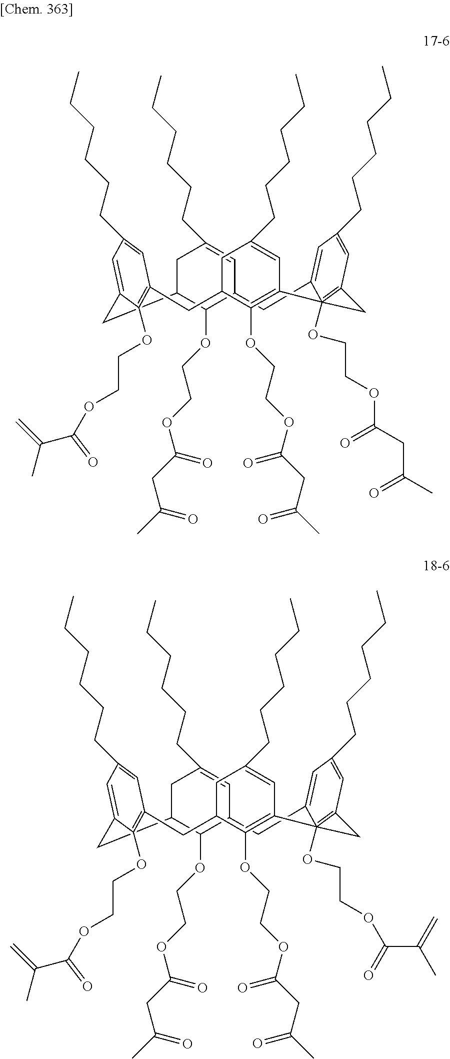

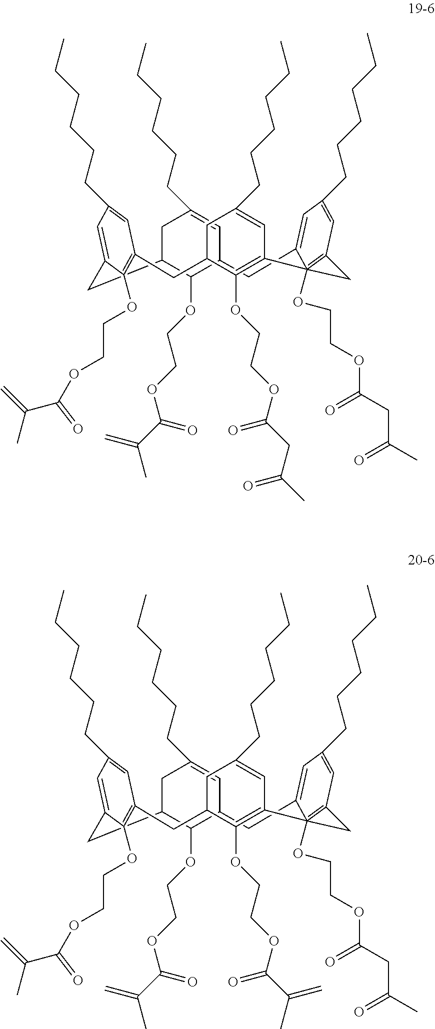

### Example 10

The same procedure as in Example 1 was repeated except that G-4 was used instead of D-6, to thereby obtain 0.399 g of the target compound 17-4. The yield was 28.7%. 0.218 g of 18-4 was obtained. The yield was 15.9%. 0.218 g of 19-4 was obtained. The yield was 15.9%. 0.114 g of 20-4 was obtained. The yield was 8.44%.

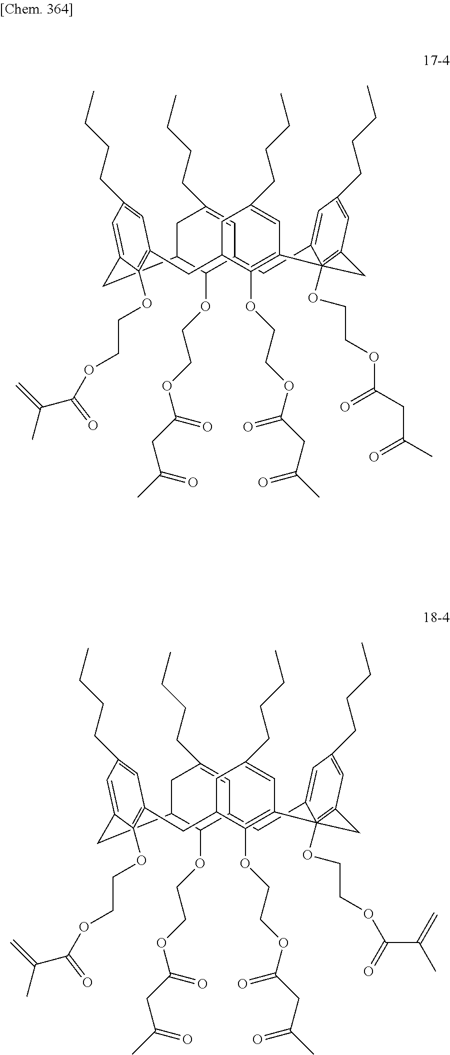

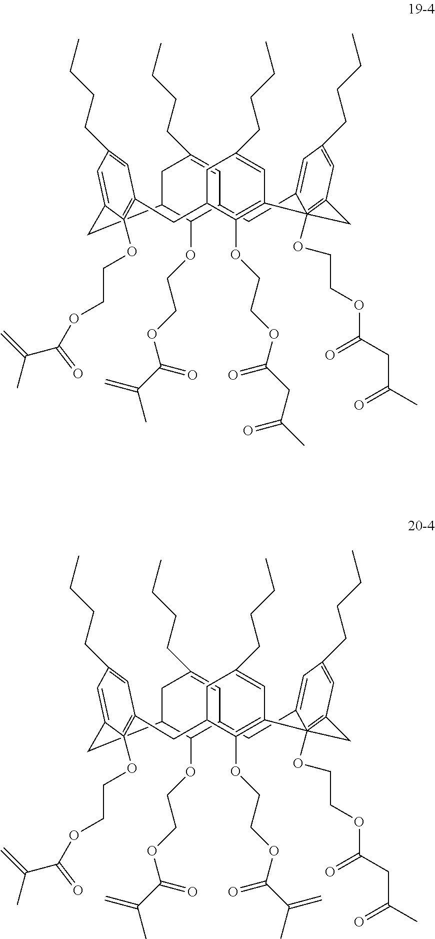

### Example 11

The same procedure as in Example 1 was repeated except that G-7 was used instead of D-6, to thereby obtain 0.415 g of the target compound 17-7. The yield was 31.4%. 0.227 g of 18-7 was obtained. The yield was 17.4%. 0.204 g of 19-7 was obtained. The yield was 15.6%. 0.123 g of 20-7 was obtained. The yield was 9.53%.

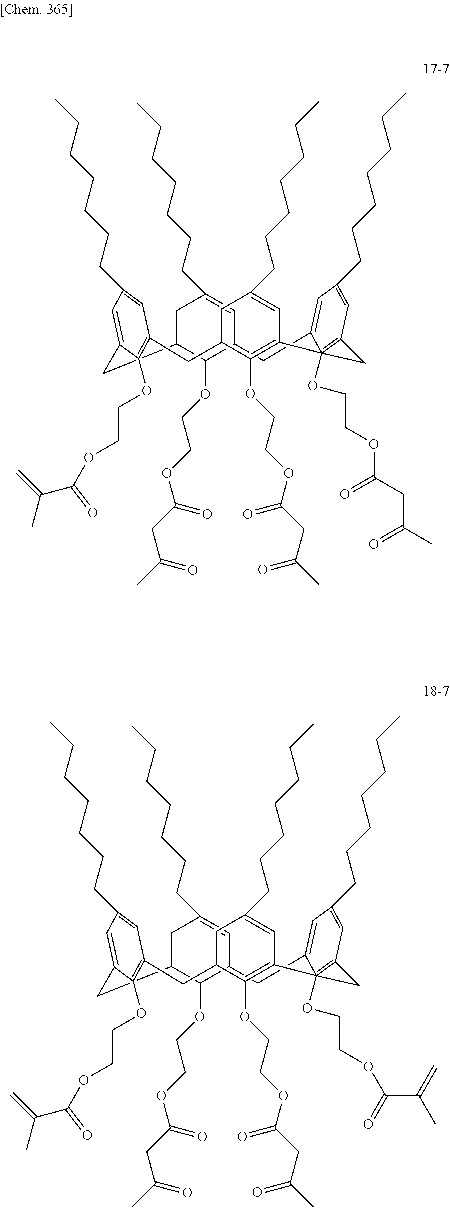

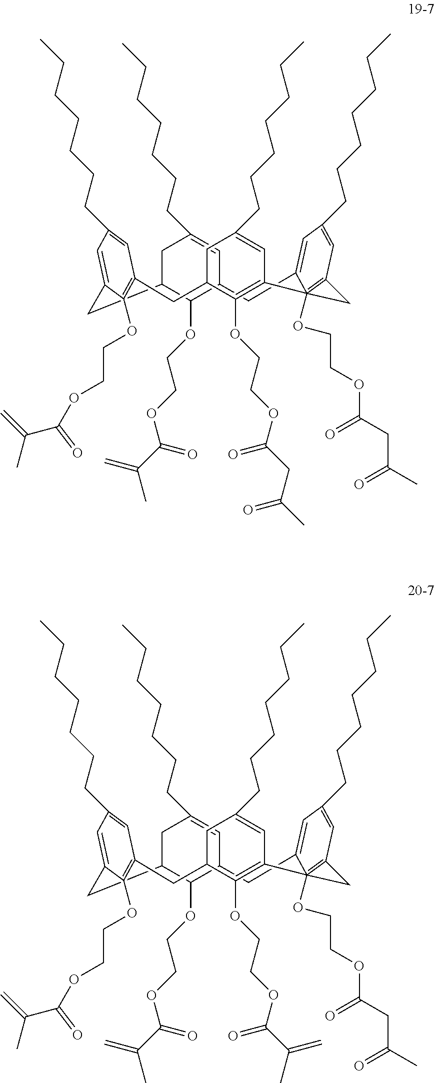

### Example 12

The same procedure as in Example 1 was repeated except that G-18 was used instead of D-6, to thereby obtain 0.374 g of the target compound 17-18. The yield was 31.2%. 0.218 g of 18-18 was obtained. The yield was 18.3%. 0.207 g of 19-18 was obtained. The yield was 17.4%. 0.107 g of 20-18 was obtained. The yield was 9.08%.

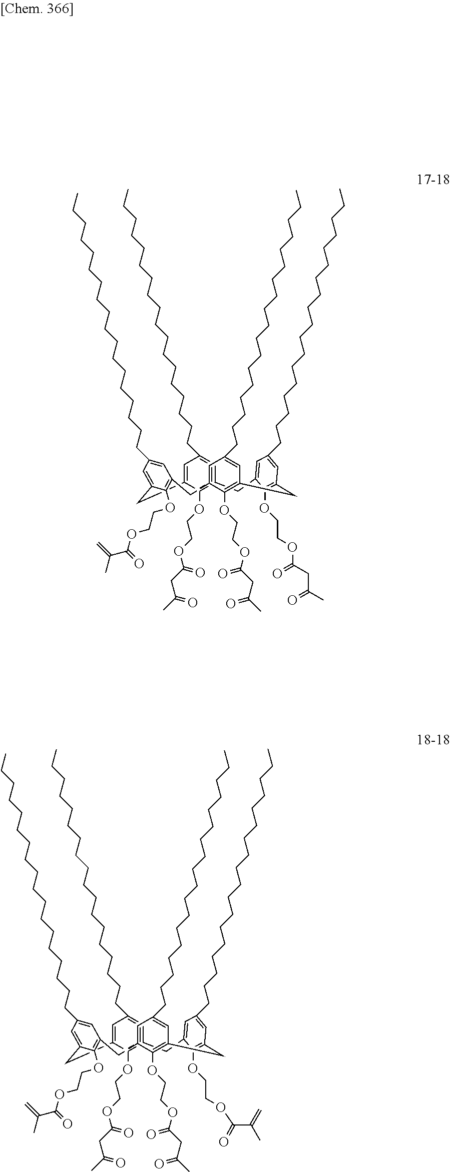 [SMILES: C=C(C)C(=O)OCCOc1c2cc(CCCCCCCCCCCCCCCCCC)cc1Cc1cc(CCCCCCCCCCCCCCCCCC)cc(c1OCCOC(=O)CC(C)=O)Cc1cc(CCCCCCCCCCCCCCCCCC)cc(c1OCCOC(=O)CC(C)=O)Cc1cc(CCCCCCCCCCCCCCCCCC)cc(c1OCCOC(=O)CC(C)=O)C2 ; C=C(C)C(=O)OCCOc1c2cc(CCCCCCCCCCCCCCCCCC)cc1Cc1cc(CCCCCCCCCCCCCCCCCC)cc(c1OCCOC(=O)CC(C)=O)Cc1cc(CCCCCCCCCCCCCCCCCC)cc(c1OCCOC(=O)C(=C)C)Cc1cc(CCCCCCCCCCCCCCCCCC)cc(c1OCCOC(=O)CC(C)=O)C2]

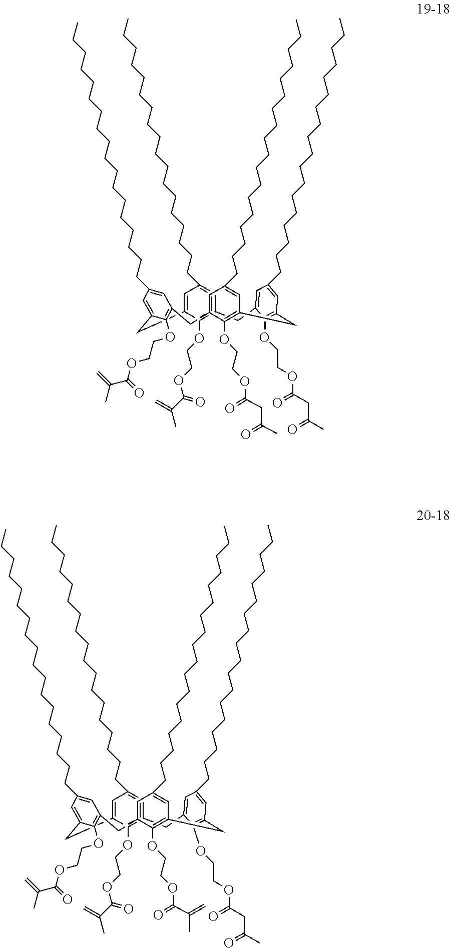 [SMILES: C=C(C)C(=O)OCCOc1c2cc(CCCCCCCCCCCCCCCCCC)cc1Cc1cc(CCCCCCCCCCCCCCCCCC)cc(c1OCCOC(=O)C(=C)C)Cc1cc(CCCCCCCCCCCCCCCCCC)cc(c1OCCOC(=O)CC(C)=O)Cc1cc(CCCCCCCCCCCCCCCCCC)cc(c1OCCOC(=O)CC(C)=O)C2 ; C=C(C)C(=O)OCCOc1c2cc(CCCCCCCCCCCCCCCCCC)cc1Cc1cc(CCCCCCCCCCCCCCCCCC)cc(c1OCCOC(=O)C(=C)C)Cc1cc(CCCCCCCCCCCCCCCCCC)cc(c1OCCOC(=O)C(=C)C)Cc1cc(CCCCCCCCCCCCCCCCCC)cc(c1OCCOC(=O)CC(C)=O)C2]

### Example 13

The same procedure as in Example 1 was repeated except that G-1 was used instead of D-6, to thereby obtain 0.334 g of the target compound 17-1. The yield was 22.5%. 0.186 g of 18-1 was obtained. The yield was 12.7%. 0.175 g of 19-1 was obtained. The yield was 12.0%. 0.102 g of 20-1 was obtained. The yield was 7.09%.

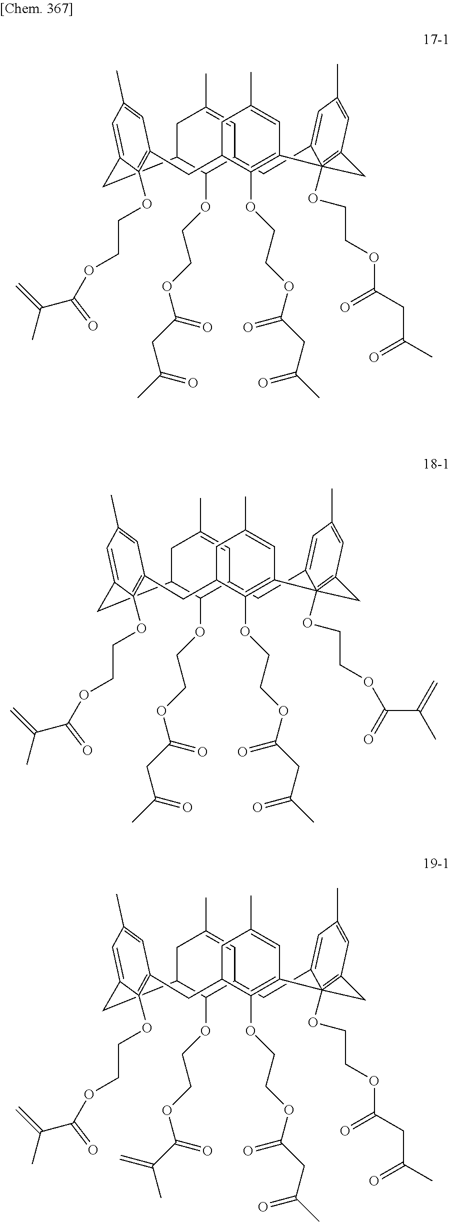

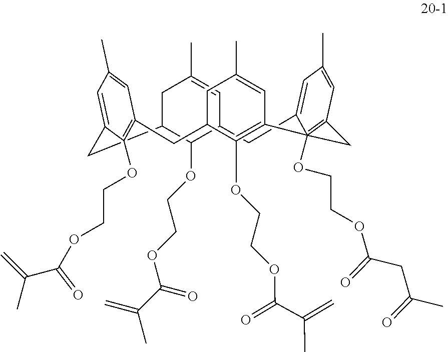

### Example 14

The same procedure as in Example 9 was repeated except that acrylic acid was used instead of methacrylic acid, to thereby obtain 0.422 g of the target compound 21-6. The yield was 31.8%. 0.214 g of 22-6 was obtained. The yield was 16.5%. 0.207 g of 23-6 was obtained. The yield was 16.0%. 0.119 g of 24-6 was obtained. The yield was 9.42%.

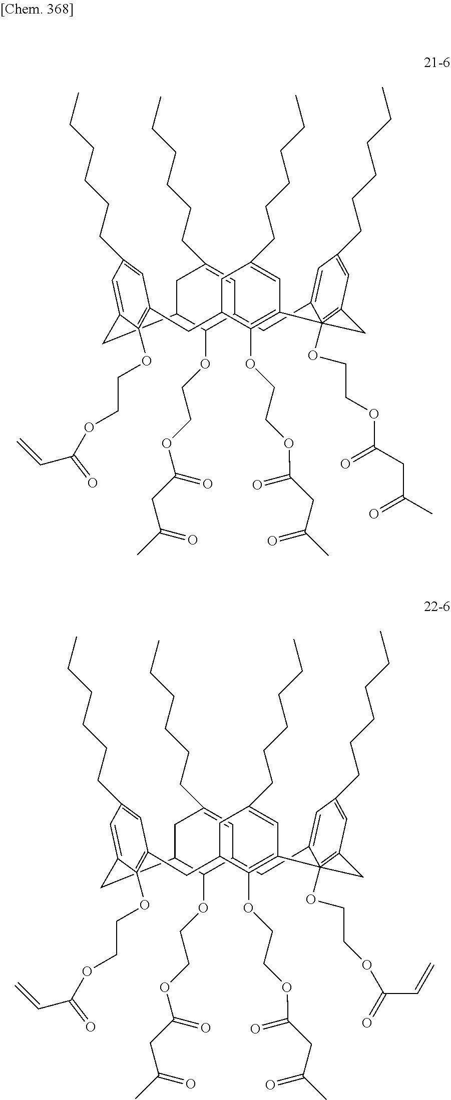

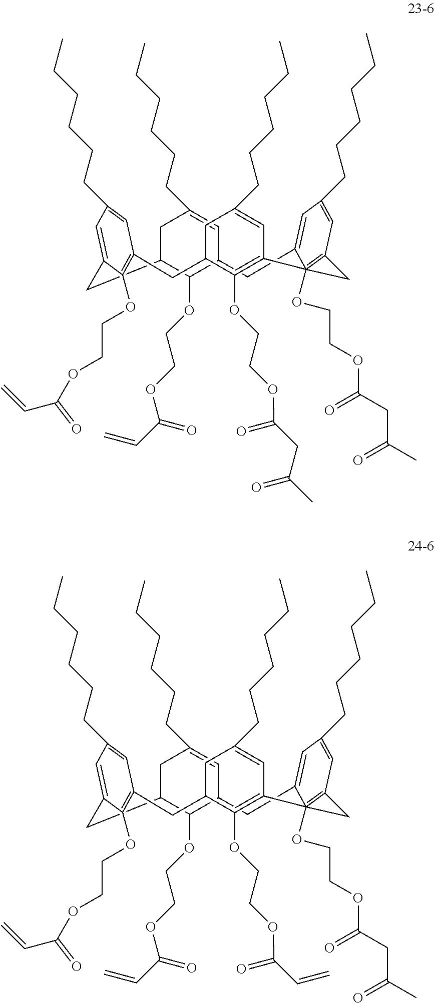

### Example 15

The same procedure as in Example 9 was repeated except that 3-oxopentanoic acid was used instead of 2-acetylacetic acid, to thereby obtain 0.402 g of the target compound 25-6. The yield was 29.0%. 0.205 g of 26-6 was obtained. The yield was 15.1%. 0.214 g of 27-6 was obtained. The yield was 15.8%. 0.114 g of 28-6 was obtained. The yield was 8.62%.

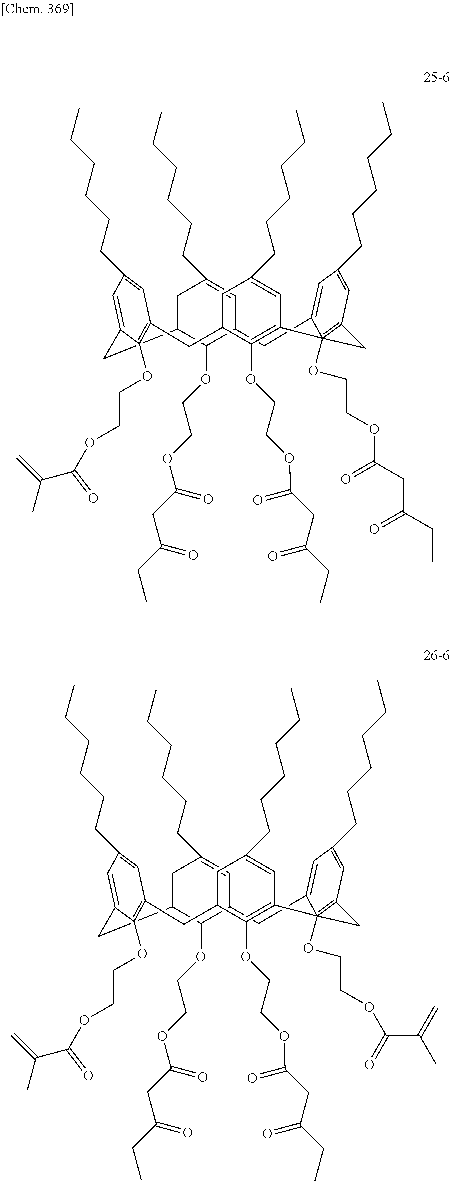

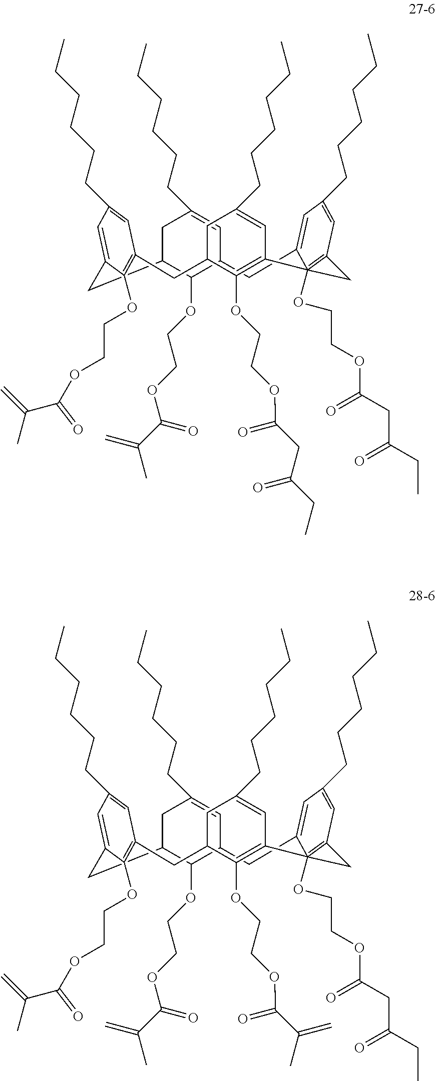

### Example 16

The same procedure as in Example 15 was repeated except that acrylic acid was used instead of methacrylic acid, to thereby obtain 0.397 g of the target compound 29-6. The yield was 28.9%. 0.216 g of 30-6 was obtained. The yield was 16.3%. 0.219 g of 31-6 was obtained. The yield was 16.5%. 0.121 g of 32-6 was obtained. The yield was 9.47%.

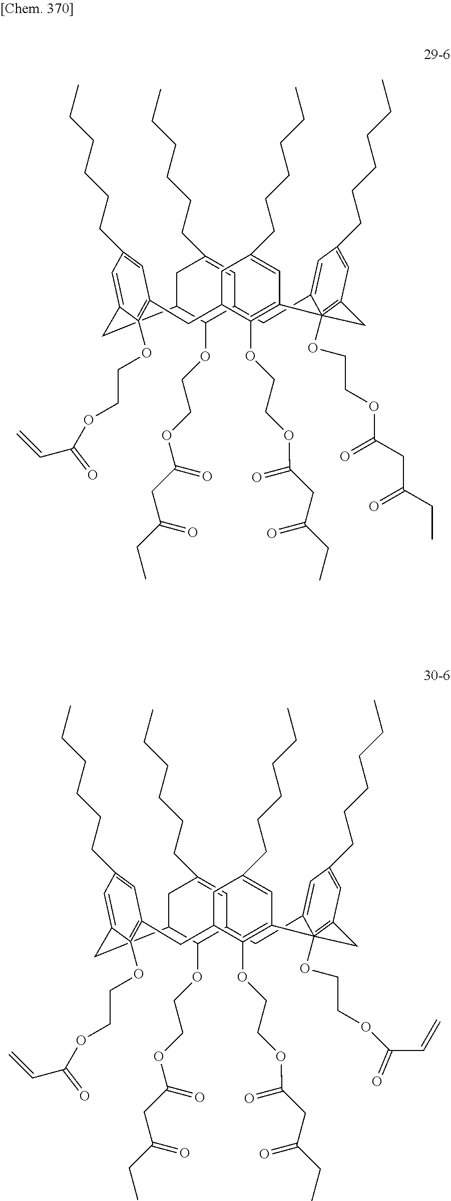

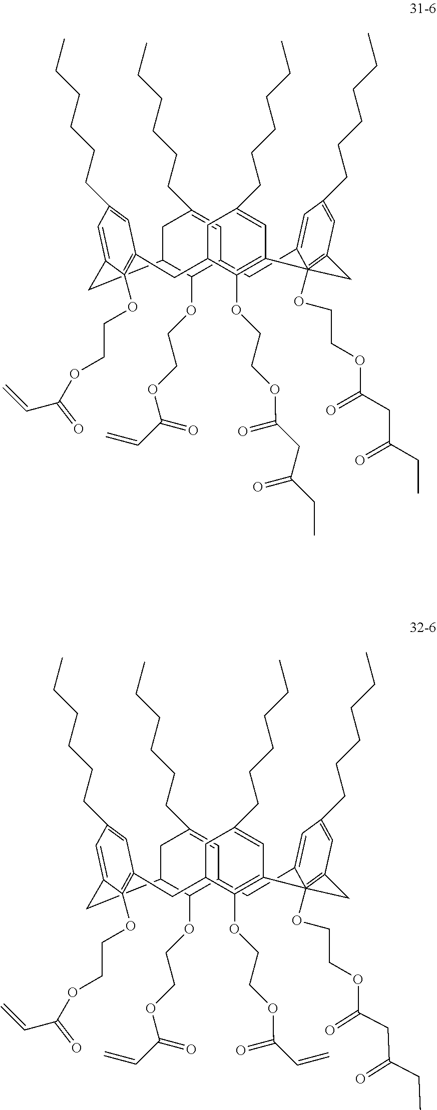

### Example 17

The same procedure as in Example 9 was repeated except that 2,2-dimethyl-3-oxobutanoic acid was used instead of 2-acetylacetic acid, to thereby obtain 0.412 g of the target compound 33-6. The yield was 28.8%. 0.234 g of 34-6 was obtained. The yield was 16.9%. 0.227 g of 35-6 was obtained. The yield was 16.4%. 0.109 g of 36-6 was obtained. The yield was 8.15%.

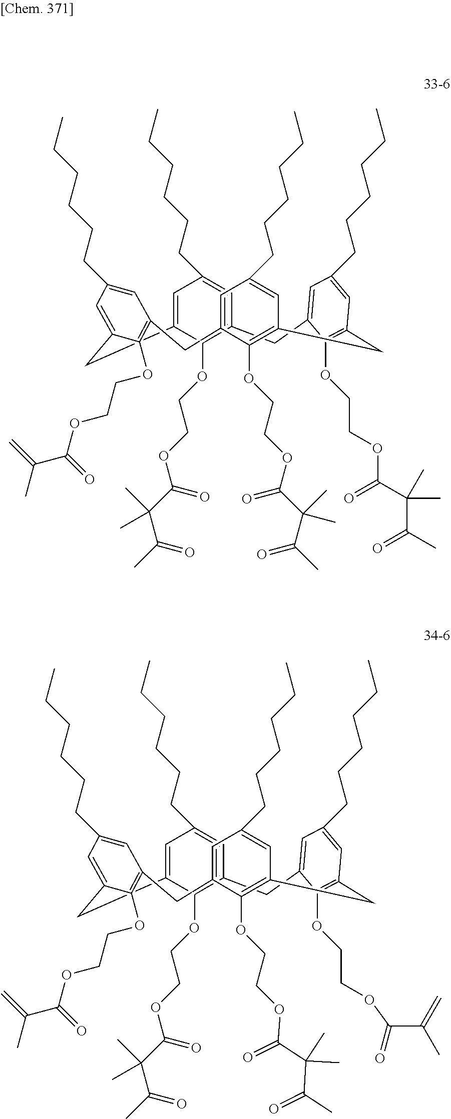

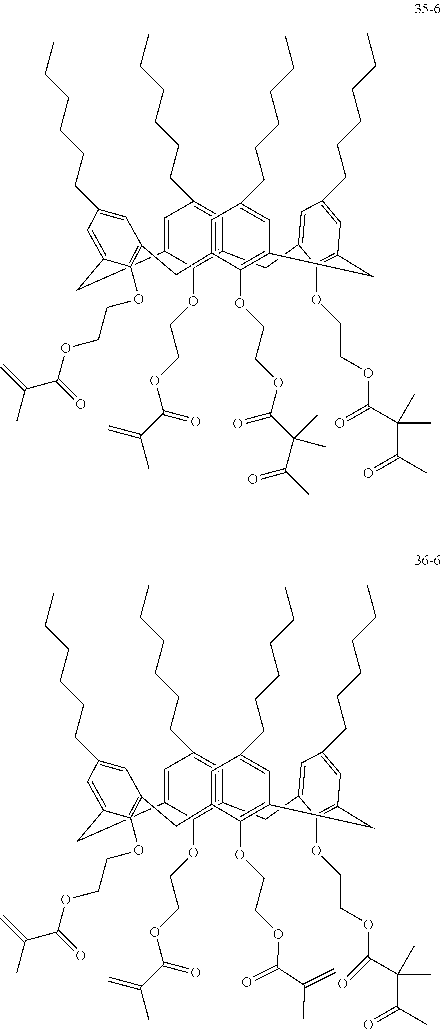

### Example 18

The same procedure as in Example 9 was repeated except that 2-oxocyclopentanecarboxylic acid was used instead of 2-acetylacetic acid, to thereby obtain 0.312 g of the target compound 37-6. The yield was 21.8%. 0.204 g of 38-6 was obtained. The yield was 14.7%. 0.197 g of 39-6 was obtained. The yield was 14.2%. 0.087 g of 40-6 was obtained. The yield was 6.50%.

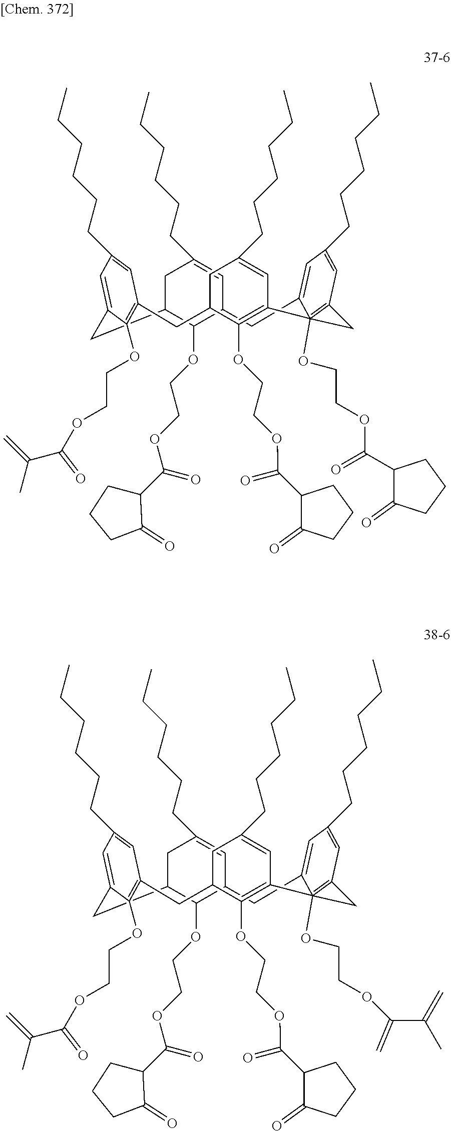

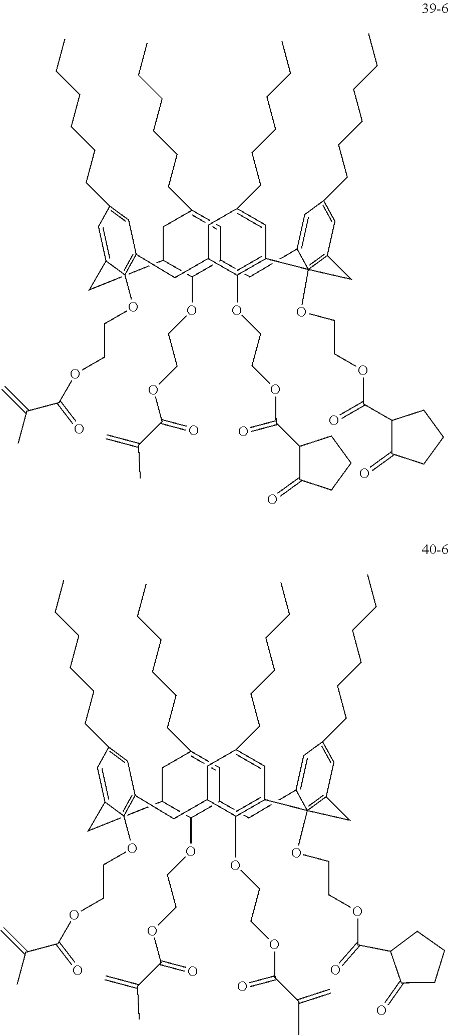

### Synthesis Example 28

The same procedure as in Synthesis Example 18 was repeated except that methyl bromopyopionate was used instead of methyl bromoacetate, to thereby obtain 4.89 g of the target compound H-6. The yield was 67.3%.

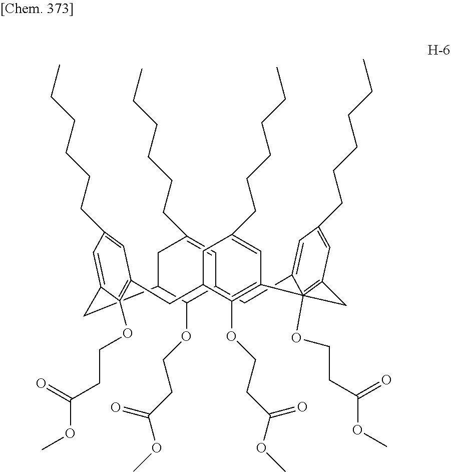

### Synthesis Example 29

The same procedure as in Synthesis Example 10 was repeated except that H-6 was used instead of C-6, to thereby obtain 3.88 g of the target compound I-6. The yield was 88.3%.

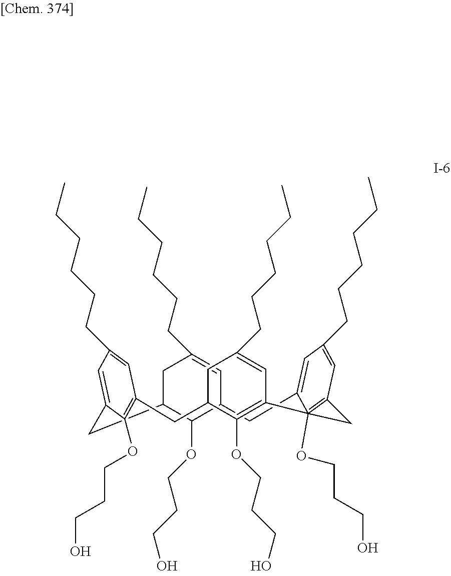

### Example 19

The same procedure as in Example 1 was repeated except that I-6 was used instead of D-6, to thereby obtain 0.331 g of the target compound 41-6. The yield was 25.0%. 0.231 g of 42-6 was obtained. The yield was 17.5%. 0.231 g of 43-6 was obtained. The yield was 17.7%. 0.129 g of 44-6 was obtained. The yield was 10.0%.

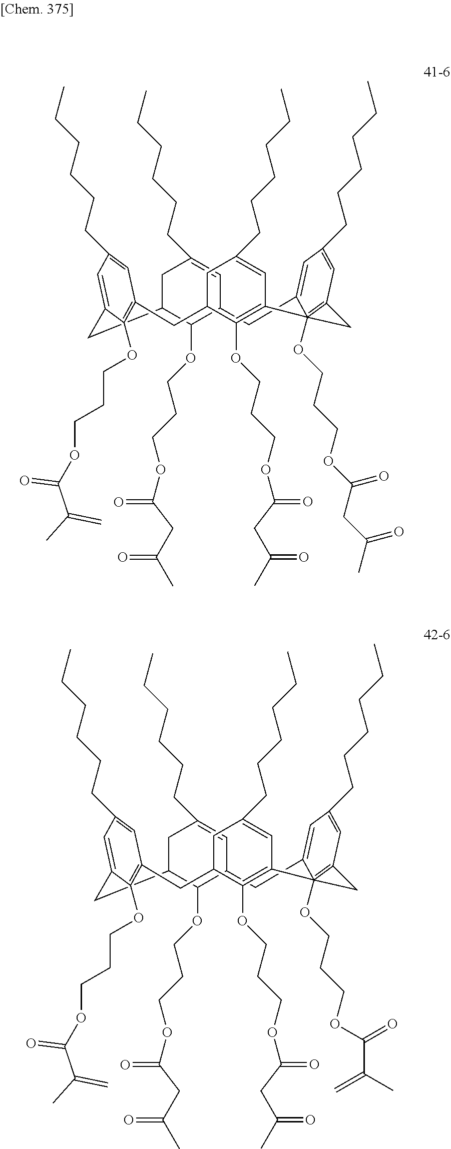

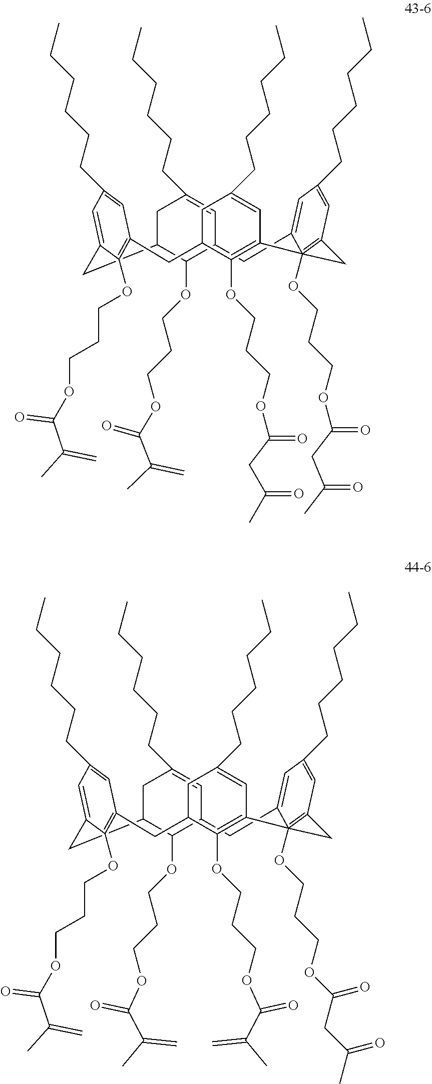

### Example 20

The same procedure as in Example 19 was repeated except that acrylic acid was used instead of methacrylic acid, to thereby obtain 0.328 g of the target compound 45-6. The yield was 25.1%. 0.214 g of 46-6 was obtained. The yield was 16.7%. 0.226 g of 47-6 was obtained. The yield was 17.7%. 0.131 g of 48-6 was obtained. The yield was 10.5%.

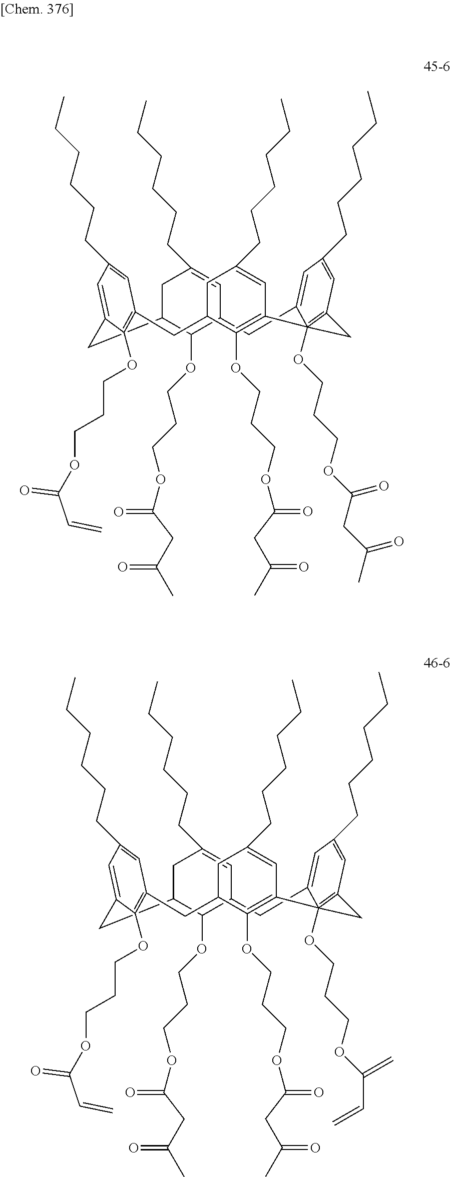

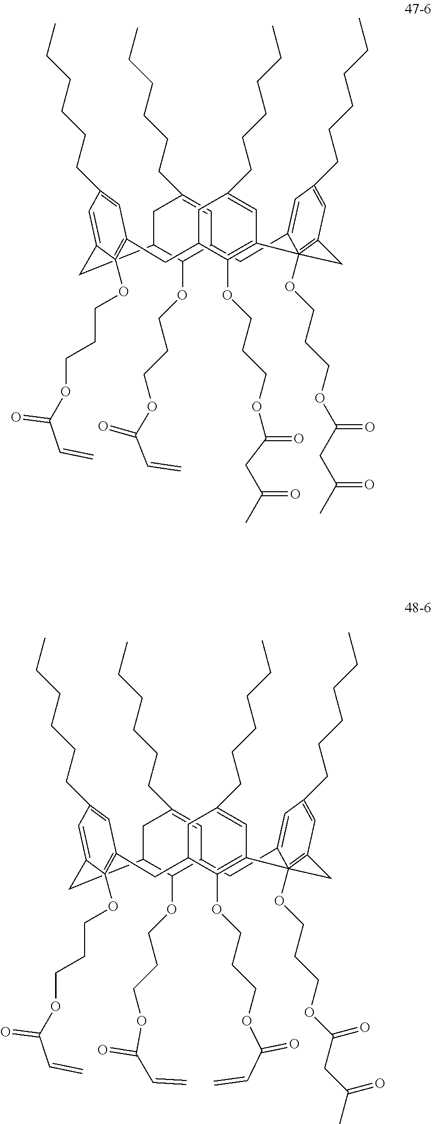

### Example 21

The same procedure as in Example 19 was repeated except that 3-oxopentanoic acid was used instead of 2-acetylacetic acid, to thereby obtain 0.318 g of the target compound 49-6. The yield was 23.3%. 0.208 g of 50-6 was obtained. The yield was 15.6%. 0.217 g of 51-6 was obtained. The yield was 16.3%. 0.106 g of 52-6 was obtained. The yield was 8.13%.

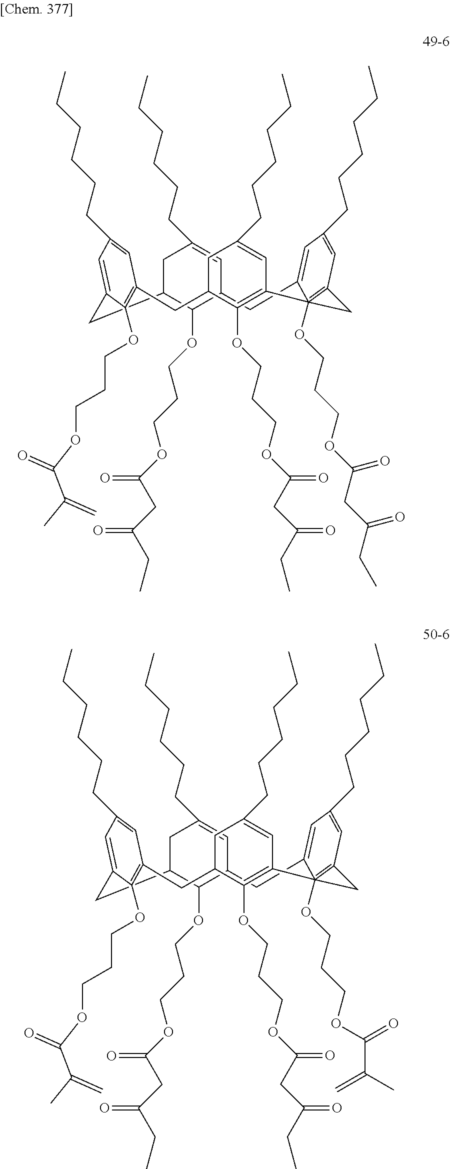

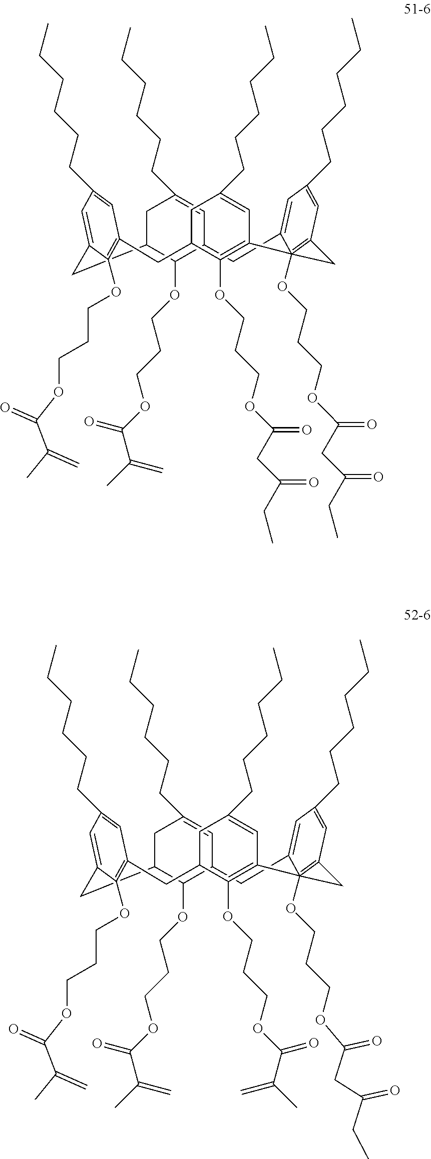

### Example 22

The same procedure as in Example 23 was repeated except that acrylic acid was used instead of methacrylic acid, to thereby obtain 0.301 g of the target compound 53-6. The yield was 22.3%. 0.221 g of 54-6 was obtained. The yield was 16.9%. 0.218 g of 55-6 was obtained. The yield was 16.7%. 0.128 g of 56-6 was obtained. The yield was 10.1%.

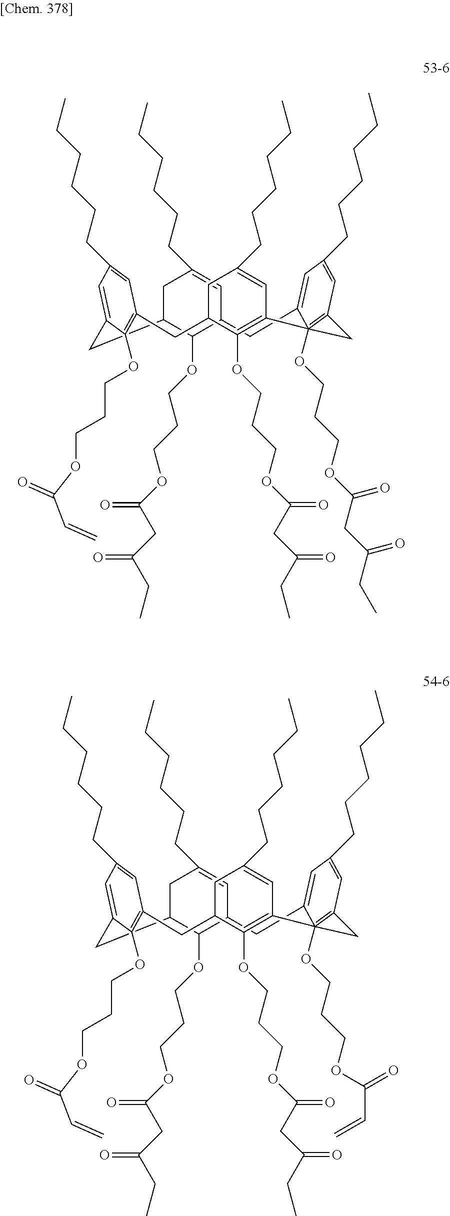

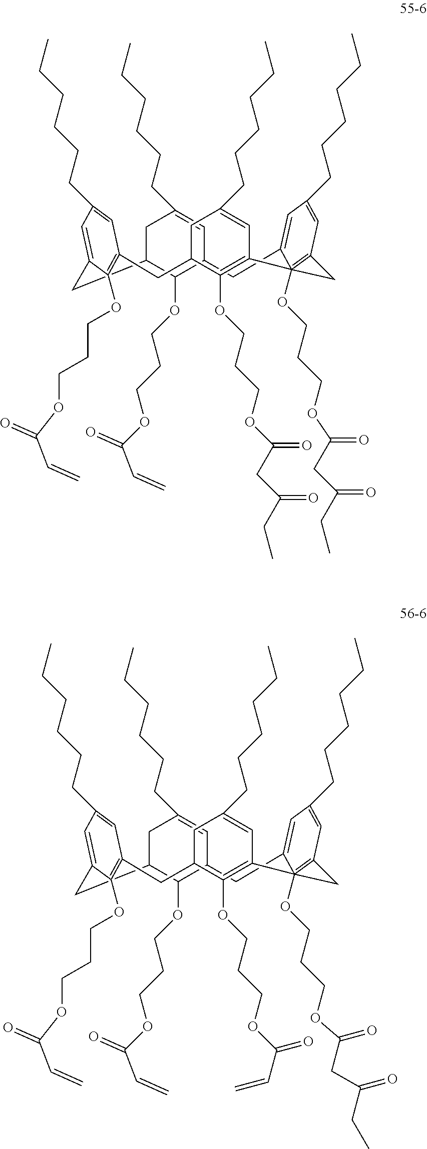

### Synthesis Example 30

A 50 mL four-necked flask equipped with a stirrer, a thermometer, and a reflux condenser tube was charged with 2.00 g (2.424 mmol) of G-6, 10.00 g of tetrahydrofuran, 1.2716 g (4.848 mmol) of triphenylphosphine, and 1.024 g (4.732 mmol) of 2-[[[(1,1-dimethylethyl)dimethylsilyl]oxy]-2-propenoic acid, and the mixture was stirred. Then 0.9803 g (4.848 mmol) of diisopropyl azodicarboxylate was added dropwise to the mixture placed in an ice bath over 30 minutes. The resulting light yellow transparent reaction solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:acetone=95:5) to thereby obtain a light yellow transparent liquid. The solvent was concentrated, and chloroform/methanol were added to reprecipitate crystals. The white crystals were filtered on a Kiriyama funnel, and the resulting white crystals were vacuum dried (at 60° C. for 6 hours or longer) to thereby obtain 1.891 g of the target compound J-6. The yield was 48.2%.

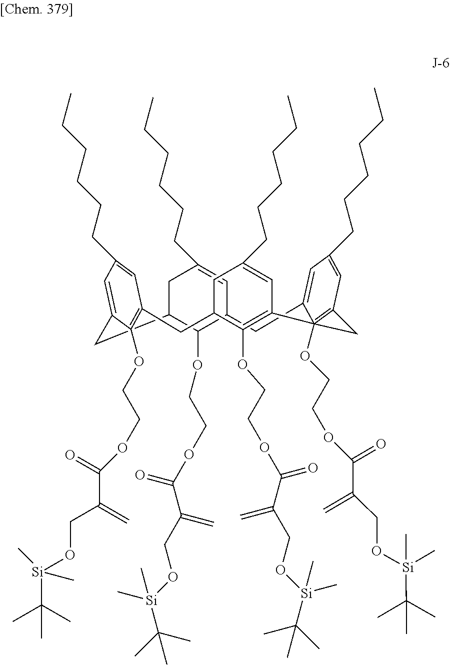

### Synthesis Example 31

The same procedure as in Synthesis Example 30 was repeated except that G-4 was used instead of G-6, to thereby obtain 1.641 g of the target compound J-4. The yield was 57.3%.

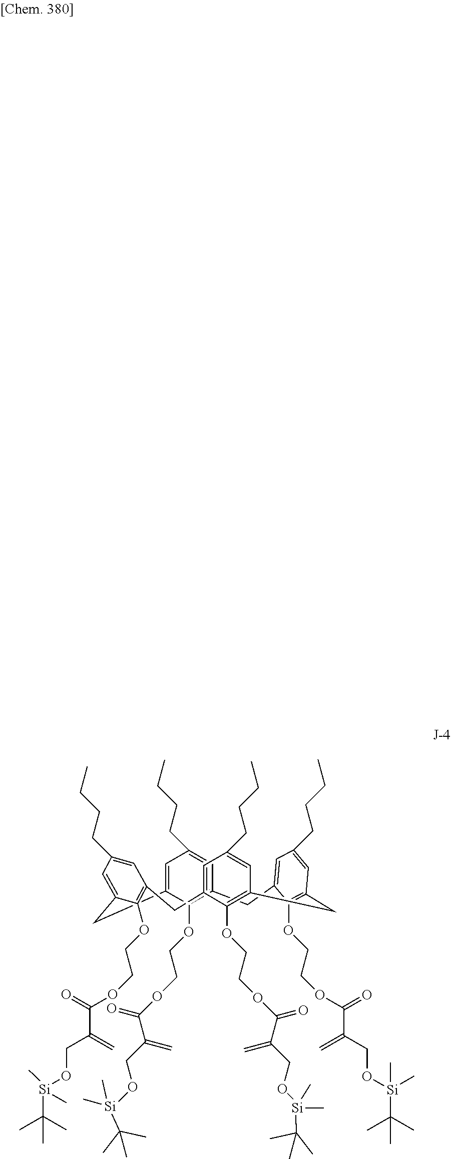 [SMILES: C=C(CO[Si](C)(C)C(C)(C)C)C(=O)OCCOc1c2cc(CCCC)cc1Cc1cc(CCCC)cc(c1OCCOC(=O)C(=C)CO[Si](C)(C)C(C)(C)C)Cc1cc(CCCC)cc(c1OCCOC(=O)C(=C)CO[Si](C)(C)C(C)(C)C)Cc1cc(CCCC)cc(c1OCCOC(=O)C(=C)CO[Si](C)(C)C(C)(C)C)C2]

### Synthesis Example 32

The same procedure as in Synthesis Example 30 was repeated except that G-7 was used instead of G-6, to thereby obtain 1.880 g of the target compound J-7. The yield was 79.0%.

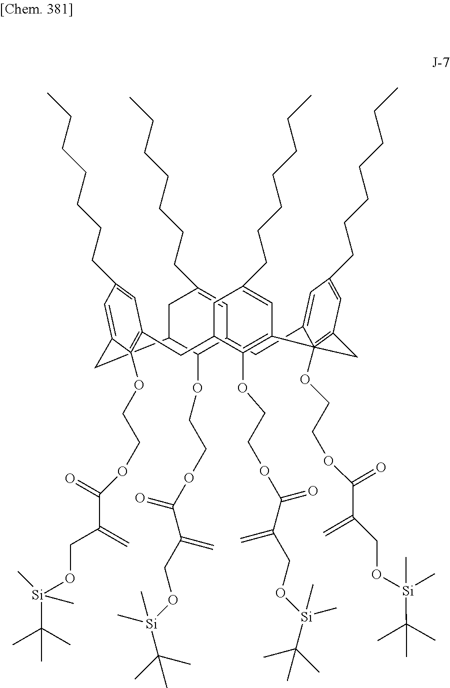

### Synthesis Example 33

The same procedure as in Synthesis Example 30 was repeated except that G-18 was used instead of G-6, to thereby obtain 2.132 g of the target compound J-18. The yield was 71.4%.

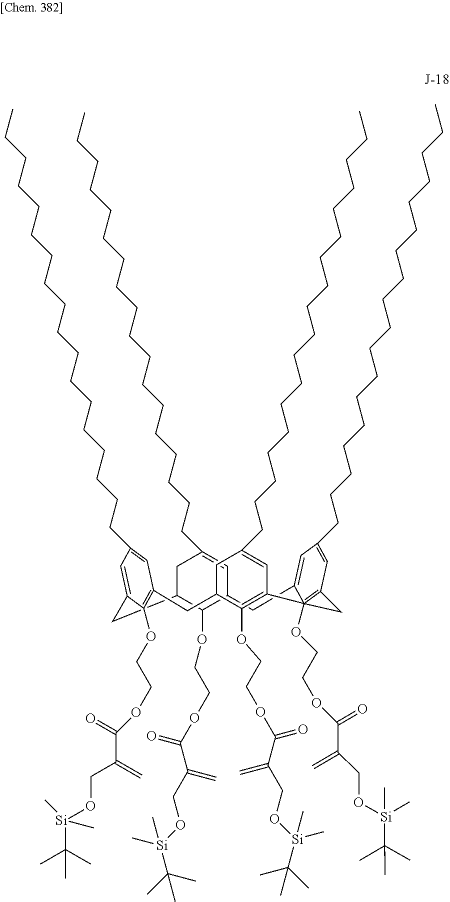

### Synthesis Example 34

The same procedure as in Synthesis Example 30 was repeated except that G-1 was used instead of G-6, to thereby obtain 1.762 g of the target compound J-1. The yield was 39.9%.

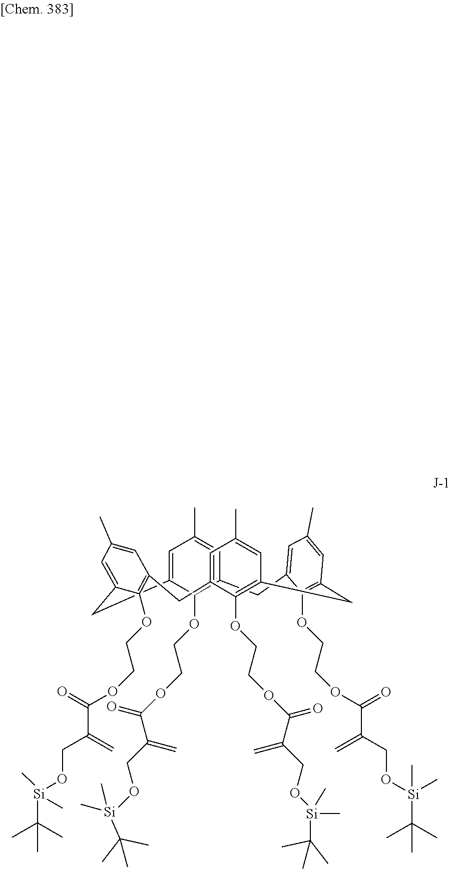 [SMILES: C=C(CO[Si](C)(C)C(C)(C)C)C(=O)OCCOc1c2cc(C)cc1Cc1cc(C)cc(c1OCCOC(=O)C(=C)CO[Si](C)(C)C(C)(C)C)Cc1cc(C)cc(c1OCCOC(=O)C(=C)CO[Si](C)(C)C(C)(C)C)Cc1cc(C)cc(c1OCCOC(=O)C(=C)CO[Si](C)(C)C(C)(C)C)C2]

### Synthesis Example 35

A 100 mL four-necked flask equipped with a stirrer, a thermometer, and a reflux condenser tube was charged with 1.891 g (1.168 mmol) of J-6, 50.00 g of tetrahydrofuran, and 0.3367 g (5.606 mmol) of acetic acid, and the mixture was stirred. Then tetrabutylammonium fluoride (an about 1 mol/L tetrahydrofuran solution 5.61 mL (5.61 mmol) was slowly added dropwise to the mixture placed in an ice bath under stirring. The resulting light yellow transparent reaction solution was stirred at room temperature for 6 hours. Ion exchanged water was added to the mixture placed in an ice bath. Then 30 g of chloroform was added, and the organic layer was separated. Next, the aqueous layer was extracted with 30 g of chloroform three times, and the extract was combined with the organic layer. The organic layer was pre-dried over anhydrous magnesium sulfate and filtered. The solvent was removed by evaporation using an evaporator to thereby obtain a red transparent liquid. The red transparent liquid was purified by column chromatography (eluent: n-hexane:acetone=95:5) to thereby obtain a light yellow transparent liquid. The solvent was concentrated, and chloroform/methanol were added to reprecipitate crystals. The white crystals were filtered on a Kiriyama funnel, and the resulting white crystals were vacuum dried (at 60° C. for 6 hours or longer) to thereby obtain 0.8451 g of the target compound K-6. The yield was 62.3%.

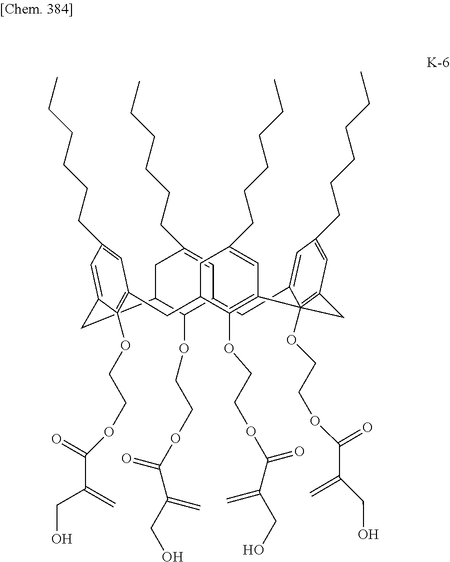

### Synthesis Example 36

The same procedure as in Synthesis Example 35 was repeated except that J-4 was used instead of J-6, to thereby obtain 0.639 g of the target compound K-4. The yield was 54.3%.

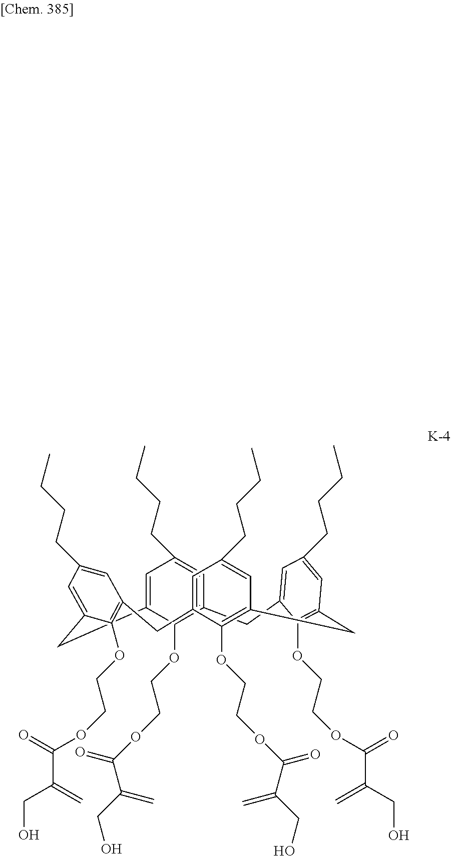 [SMILES: C=C(CO)C(=O)OCCOc1c2cc(CCCC)cc1Cc1cc(CCCC)cc(c1OCCOC(=O)C(=C)CO)Cc1cc(CCCC)cc(c1OCCOC(=O)C(=C)CO)Cc1cc(CCCC)cc(c1OCCOC(=O)C(=C)CO)C2]

### Synthesis Example 37

The same procedure as in Synthesis Example 35 was repeated except that J-7 was used instead of J-6, to thereby obtain 0.873 g of the target compound K-7. The yield was 62.4%.

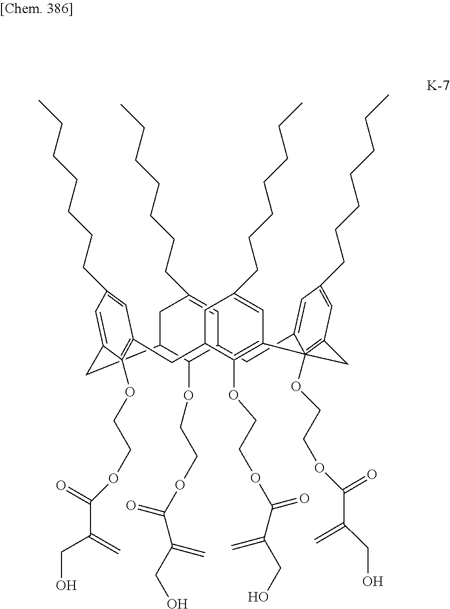

### Synthesis Example 38

The same procedure as in Synthesis Example 35 was repeated except that J-18 was used instead of J-6, to thereby obtain 1.092 g of the target compound K-18. The yield was 63.2%.

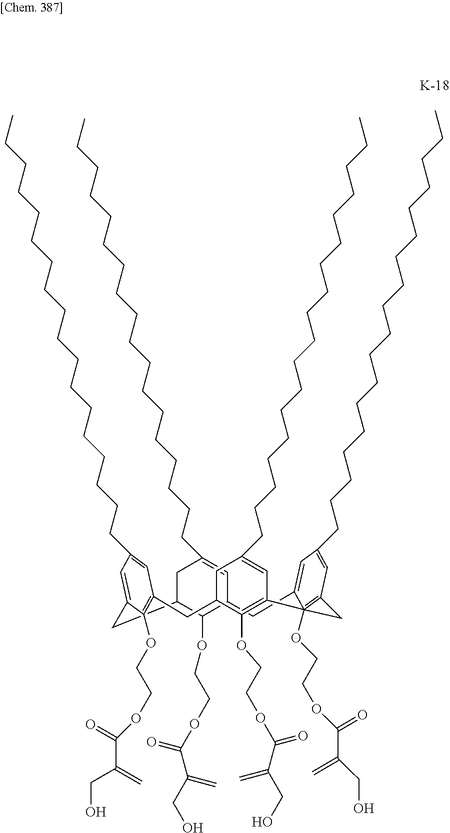

### Synthesis Example 39

The same procedure as in Synthesis Example 35 was repeated except that J-1 was used instead of J-6, to thereby obtain 0.654 g of the target compound K-1. The yield was 54.2%.

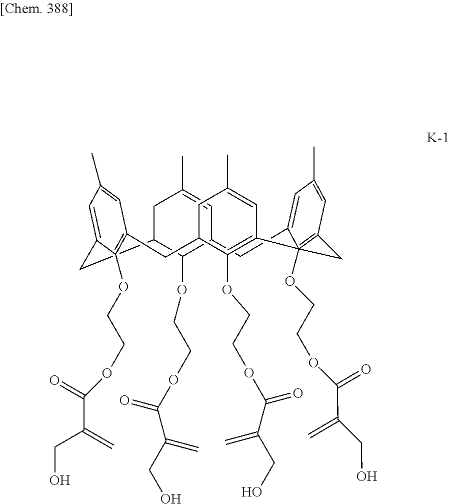

### Example 23

A 30 mL four-necked flask equipped with a stirrer, a thermometer, and a reflux condenser tube was charged with 0.300 g (0.236 mmol) of K-6, 0.679 g of tetrahydrofuran, 0.494 g (1.884 mmol) triphenylphosphine, and 0.192 g (1.884 mmol) of 2-acetylacetic acid, and the mixture was stirred. Then 0.423 g (1.884 mmol) of diisopropyl azodicarboxylate diluted with 0.340 g of tetrahydrofuran was added dropwise to the mixture placed in an ice bath over 30 minutes. The resulting light yellow transparent reaction solution was stirred at room temperature for 6 hours. Hexane was added to the reaction solution to precipitate and remove by-products such as triphenylphosphine. Then the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:ethyl acetate=85:15) to thereby obtain a light yellow transparent liquid. The solvent was concentrated, and the resulting solution was washed with methanol. The obtained colorless transparent viscous solid was vacuum dried (at 60° C. for 6 hours or longer) to thereby obtain 0.285 g of the target compound 57-6. The yield was 75.2%.

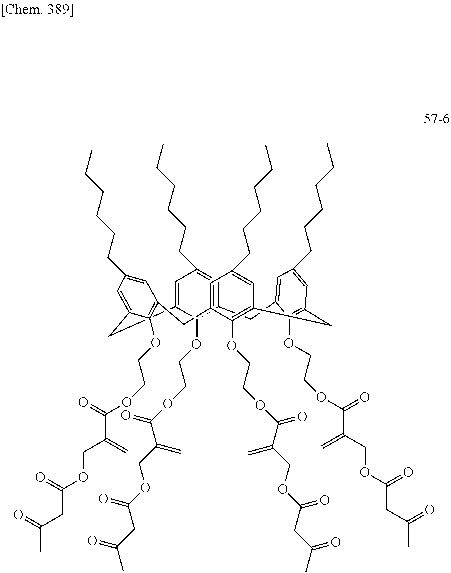 [SMILES: C=C(COC(=O)CC(C)=O)C(=O)OCCOc1c2cc(CCCCCC)cc1Cc1cc(CCCCCC)cc(c1OCCOC(=O)C(=C)COC(=O)CC(C)=O)Cc1cc(CCCCCC)cc(c1OCCOC(=O)C(=C)COC(=O)CC(C)=O)Cc1cc(CCCCCC)cc(c1OCCOC(=O)C(=C)COC(=O)CC(C)=O)C2]

### Example 24

The same procedure as in Example 23 was repeated except that K-4 was used instead of K-6, to thereby obtain 0.278 g of the target compound 57-4. The yield was 71.9%.

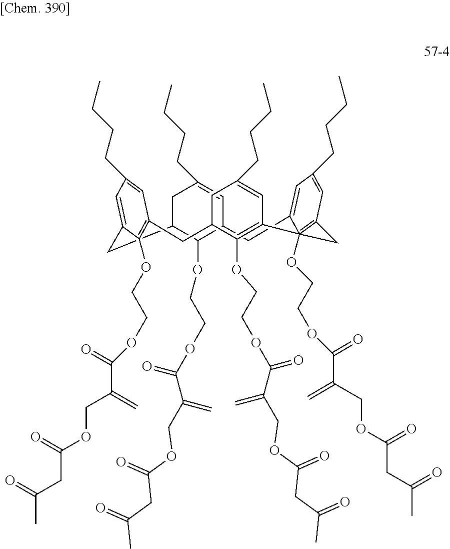

### Example 25

The same procedure as in Example 23 was repeated except that K-7 was used instead of K-6, to thereby obtain 0.293 g of the target compound 57-7. The yield was 78.0%.

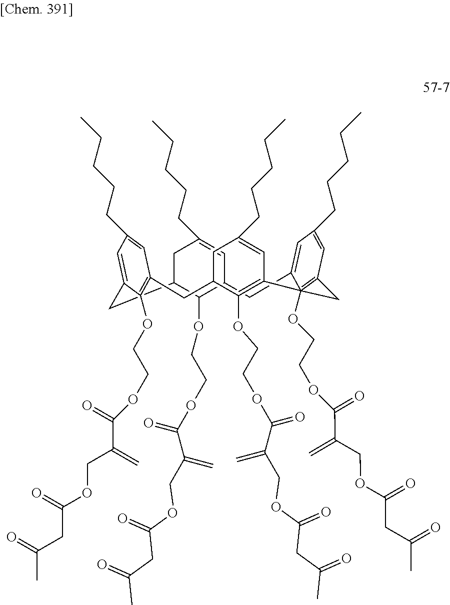

### Example 26

The same procedure as in Example 23 was repeated except that K-18 was used instead of K-6, to thereby obtain 0.301 g of the target compound 57-18. The yield was 85.6%.

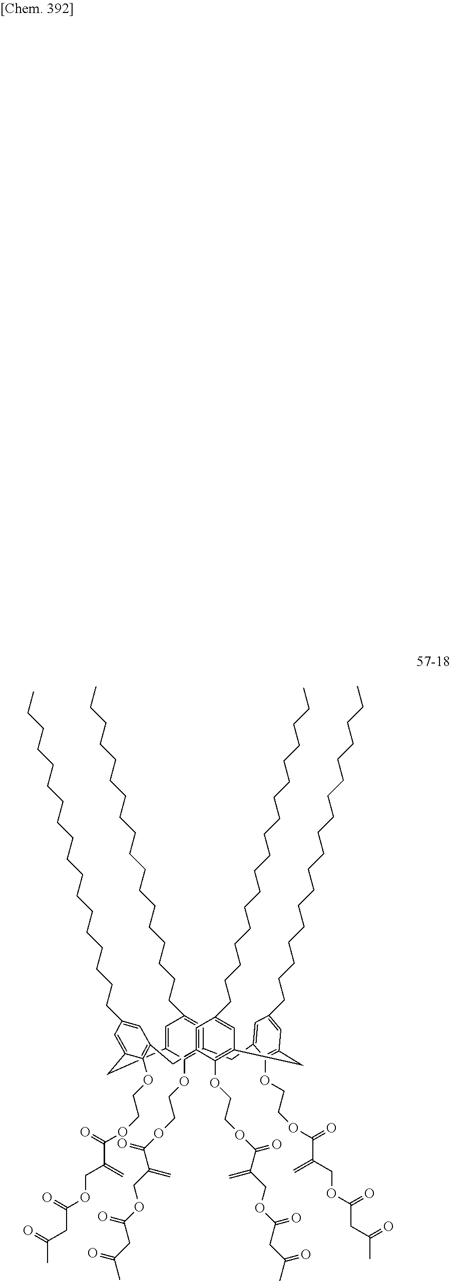 [SMILES: C=C(COC(=O)CC(C)=O)C(=O)OCCOc1c2cc(CCCCCCCCCCCCCCCCCC)cc1Cc1cc(CCCCCCCCCCCCCCCCCC)cc(c1OCCOC(=O)C(=C)COC(=O)CC(C)=O)Cc1cc(CCCCCCCCCCCCCCCCCC)cc(c1OCCOC(=O)C(=C)COC(=O)CC(C)=O)Cc1cc(CCCCCCCCCCCCCCCCCC)cc(c1OCCOC(=O)C(=C)COC(=O)CC(C)=O)C2]

### Example 27

The same procedure as in Example 23 was repeated except that K-1 was used instead of K-6, to thereby obtain 0.297 g of the target compound 57-1. The yield was 74.0%.

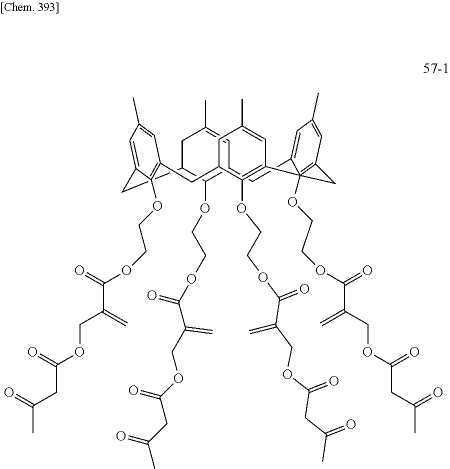

### Example 28

The same procedure as in Example 23 was repeated except that 3-oxopentanoic acid was used instead of 2-acetylacetic acid, to thereby obtain 0.312 g of the target compound 58-6. The yield was 79.5%.

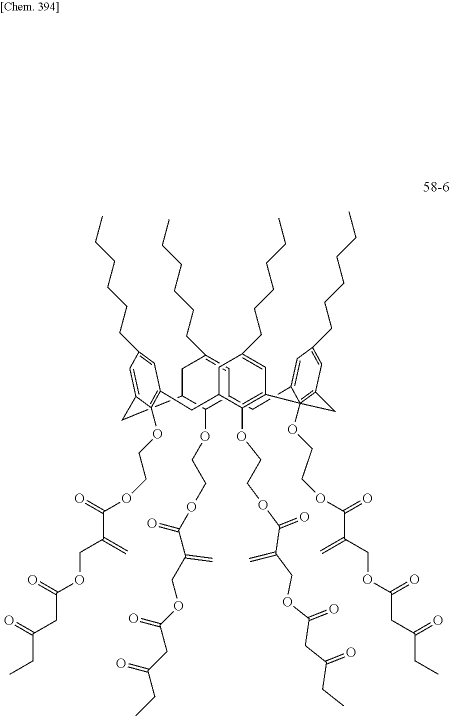

### Synthesis Example 40

The same procedure as in Synthesis Example 30 was repeated except that 4-[[[(1,1-dimethylethyl)dimethylsilyl]oxy]-2-methylenebutanoic acid was used instead of 2-[[[(1,1-dimethylethyl)dimethylsilyl]oxy]2-propenoic acid, to thereby obtain 2.420 g of the target compound L-6. The yield was 72.6%.

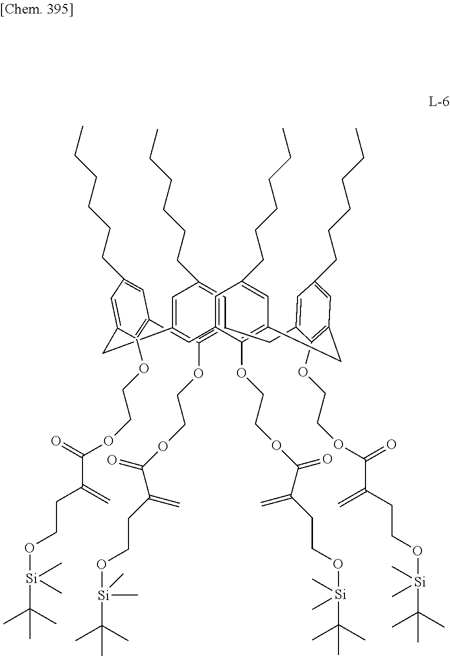 [SMILES: C=C(CCO[Si](C)(C)C(C)(C)C)C(=O)OCCOc1c2cc(CCCCCC)cc1Cc1cc(CCCCCC)cc(c1OCCOC(=O)C(=C)CCO[Si](C)(C)C(C)(C)C)Cc1cc(CCCCCC)cc(c1OCCOC(=O)C(=C)CCO[Si](C)(C)C(C)(C)C)Cc1cc(CCCCCC)cc(c1OCCOC(=O)C(=C)CCO[Si](C)(C)C(C)(C)C)C2]

### Synthesis Example 41

The same procedure as in Synthesis Example 35 was repeated except that L-6 was used instead of J-6, to thereby obtain 1.07 g of the target compound M-6. The yield was 59.4%.

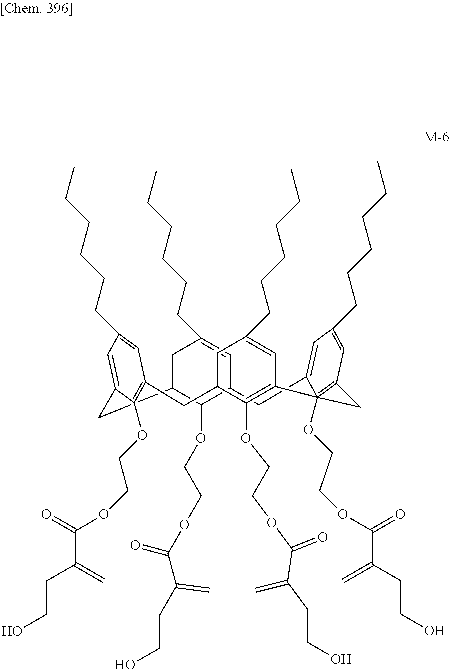

### Example 29

The same procedure as in Example 23 was repeated except that M-6 was used instead of K-6, to thereby obtain 0.292 g of the target compound 59-6. The yield was 77.7%.

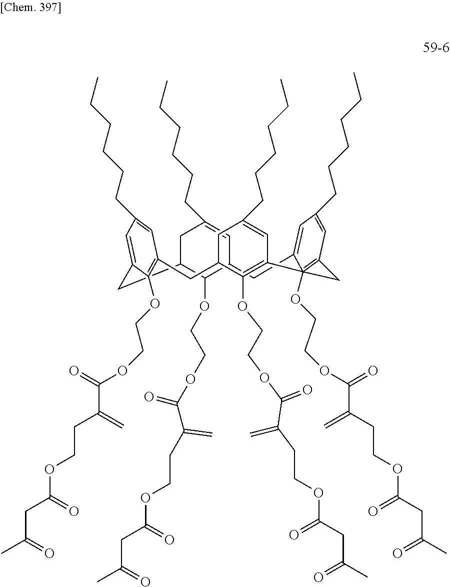

### Example 30

The same procedure as in Example 29 was repeated except that 3-oxopentanoic acid was used instead of 2-acetylacetic acid, to thereby obtain 0.318 g of the target compound 60-6. The yield was 81.8%.

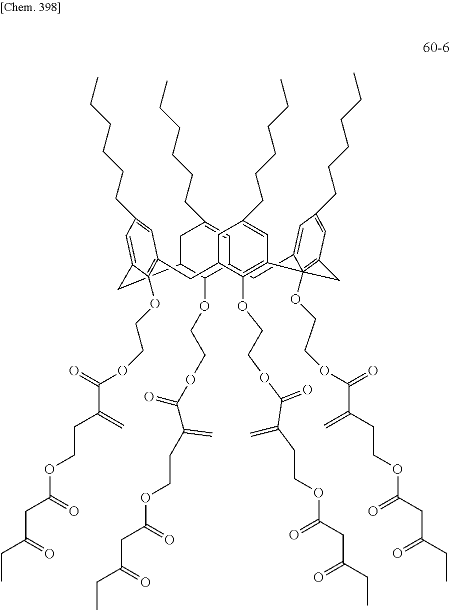

### Synthesis Example 42

A 1 L four-necked flask equipped with a stirrer, a dropping funnel, a thermometer, and a reflux condenser tube was charged with sodium hydride (7.54 g, 188.4 mmol) in a nitrogen atmosphere, and mineral oil was removed by washing with hexane. Then dry DMF (160 mL) and hexyl bromide (37.2 g, 207.4 mmol) were added, and the mixture was heated to 70° C. under stirring. A solution prepared by dissolving intermediate A (10 g, 23.6 mmol) obtained in Synthesis Example 1 in dry DMF (80 mL) was added to the mixture from a dropping funnel. After completion of the addition, the resulting mixture was continuously stirred for additional 2 hours. After cooled to room temperature, the reaction mixture was poured onto ice (300 g), and concentrated hydrochloric acid was added to acidify the aqueous solution. The resulting aqueous solution was extracted with chloroform (200 mL) twice. The chloroform solution was washed with water until the pH was 5 or higher, further washed with saturated brine, and dried over anhydrous magnesium sulfate. The solvent was removed using an evaporator to thereby obtain a yellow liquid. Methanol was added to the mixture under stirring to precipitate solids. The solids were collected by filtration and recrystallized with isopropyl alcohol. The obtained white crystals were vacuum dried to thereby obtain a compound represented by the following formula (11.6 g, yield 65%).

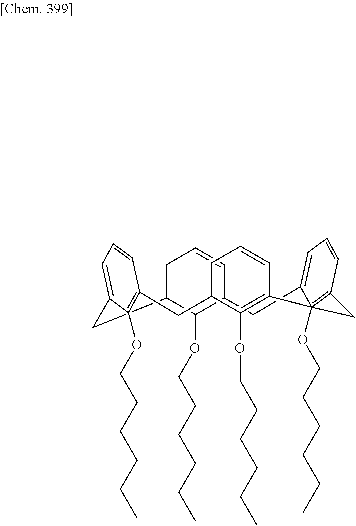

### Synthesis Example 43

The same procedure as in Synthesis Example 40 was repeated except that methyl iodide was used instead of hexyl bromide and that the reaction was performed at room temperature for 24 hours, to thereby obtain a compound represented by the following formula (6.8 g, yield 60%).

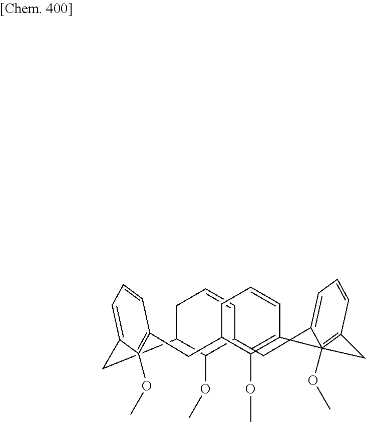

### Synthesis Example 44

The same procedure as in Synthesis Example 40 was repeated except that butyl bromide was used instead of hexyl bromide, to thereby obtain a compound represented by the following formula (11.0 g, yield 72%).

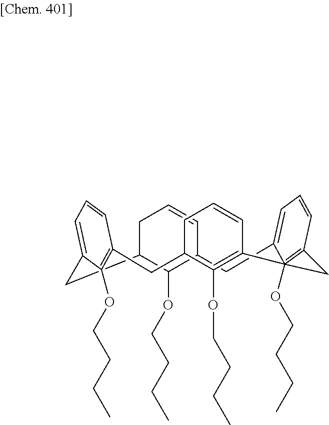

### Synthesis Example 45

The same procedure as in Synthesis Example 40 was repeated except that heptyl bromide was used instead of hexyl bromide, to thereby obtain a compound represented by the following formula (14.4 g, yield 75%).

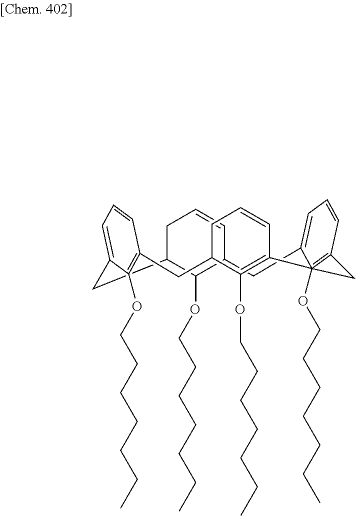

### Synthesis Example 46

The same procedure as in Synthesis Example 40 was repeated except that octadecyl bromide was used instead of hexyl bromide, to thereby obtain a compound represented by the following formula (23.6 g, yield 70%).

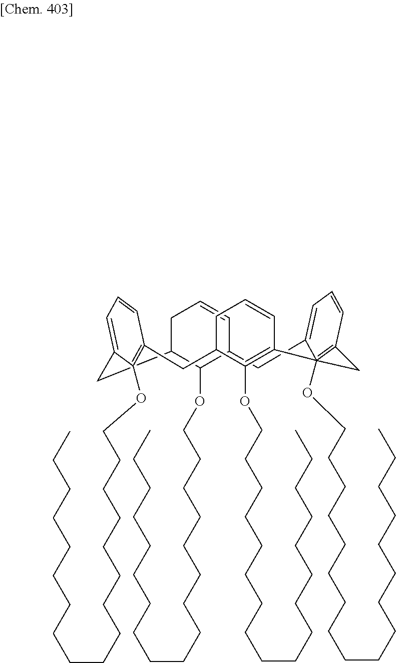

### Synthesis Example 47

Referring to a known document (Organic & Biomolecular Chemistry, 13, 1708-1723; 2015), a compound represented by the following formula was synthesized in two steps using the compound obtained in Synthesis Example 42 (5.0 g, 6.57 mmol) (yield amount 3.3 g, yield 67%).

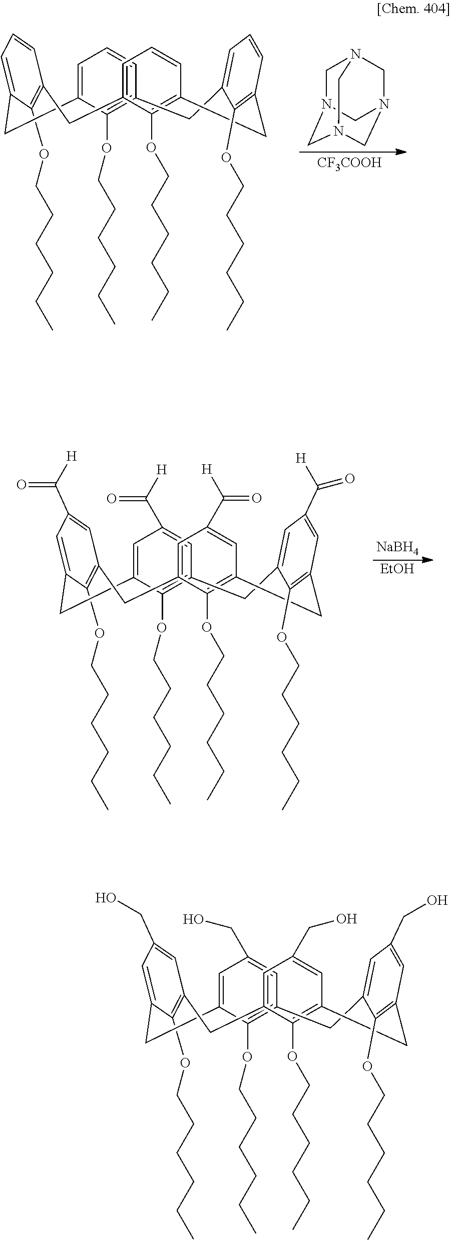

### Synthesis Example 48

The same procedure as in Synthesis Example 47 was repeated except that the compound obtained in Synthesis Example 43 (5.0 g, 10.4 mmol) was used instead of the compound obtained in Synthesis Example 42, to synthesize a compound represented by the following formula in two steps (3.75 g, yield 60%).

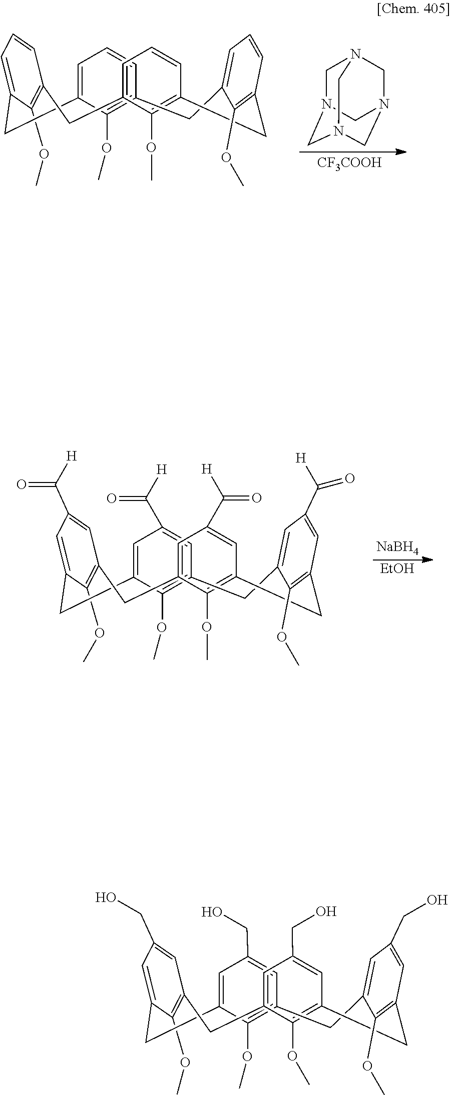

### Synthesis Example 49

The same procedure as in Synthesis Example 47 was repeated except that the compound obtained in Synthesis Example 44 (5.0 g, 7.7 mmol) was used instead of the compound obtained in Synthesis Example 42, to synthesize a compound represented by the following formula in two steps (3.73 g, yield 63%).

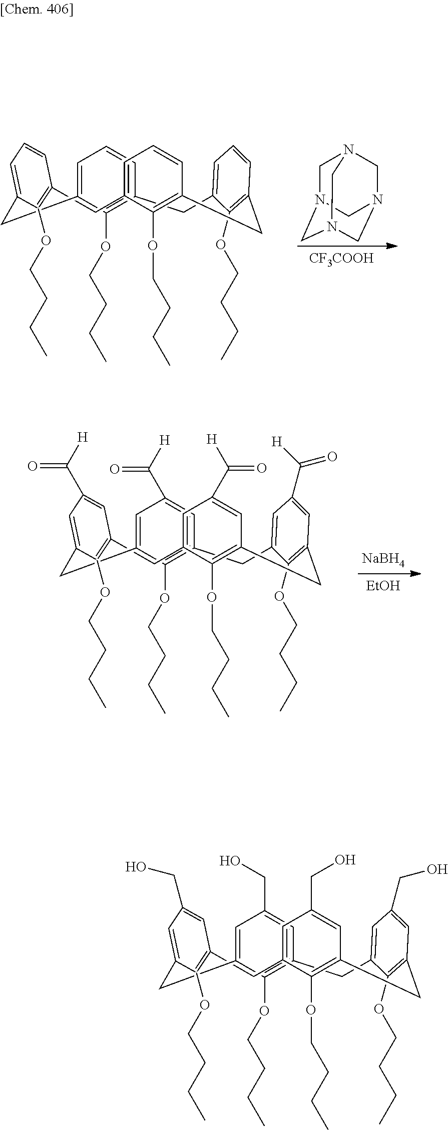

### Synthesis Example 50

The same procedure as in Synthesis Example 47 was repeated except that the compound obtained in Synthesis Example 45 (5.0 g, 6.1 mmol) was used instead of the compound obtained in Synthesis Example 42, to synthesize a compound represented by the following formula in two steps (4.01 g, yield 70%).

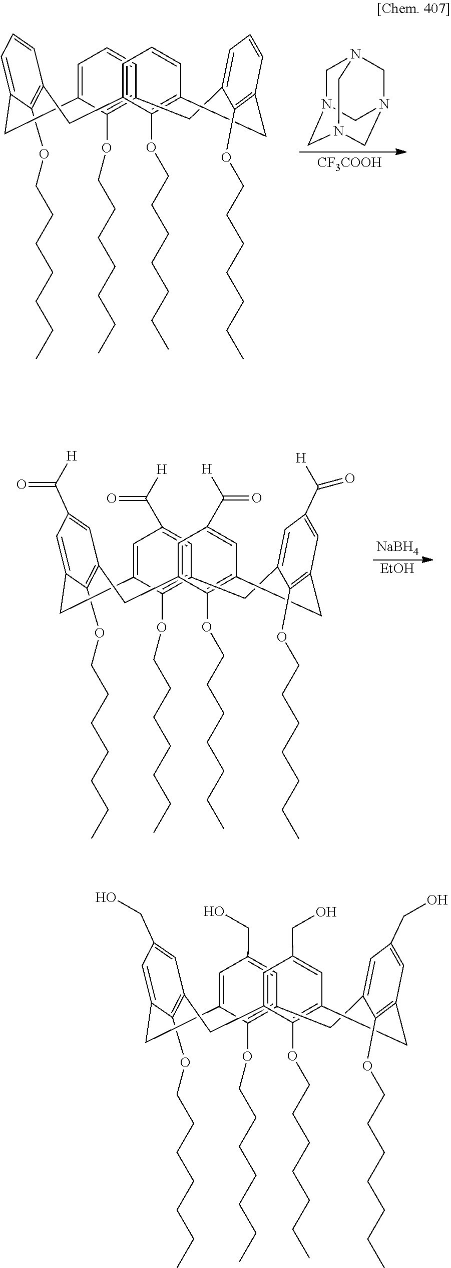

### Synthesis Example 51

The same procedure as in Synthesis Example 47 was repeated except that the compound obtained in Synthesis Example 46 (10.0 g, 7.0 mmol) was used instead of the compound obtained in Synthesis Example 42, to synthesize a compound represented by the following formula in two steps (5.96 g, yield 55%).

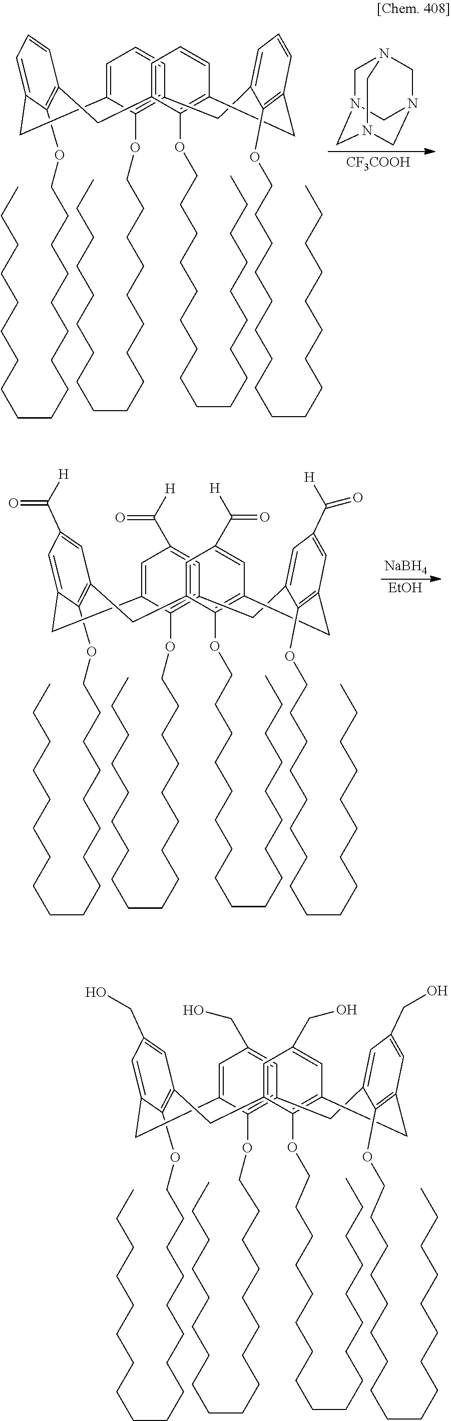

### Synthesis Example 52

A 500 mL four-necked flask equipped with a stirrer, a dropping funnel, a thermometer, and a reflux condenser tube was charged with sodium hydride (3.28 g, 82.1 mmol) in a nitrogen atmosphere, and mineral oil was removed by washing with hexane. Then dry DMF (100 mL) and hexyl bromide (16.2 g, 90.3 mmol) were added, and the mixture was heated to 70° C. under stirring. Then a solution prepared by dissolving 5,11,17,23-tetraallyl-25,26,27,28-tetrahydroxycalix[4]arene (6.0 g, 10.3 mmol) synthesized by a method described in a known document (The Journal of Organic Chemistry 50, 5802-58061; 1985) in dry DMF (40 mL) was added from a dropping funnel. After completion of the addition, the resulting mixture was continuously stirred for additional 2 hours. After cooled to room temperature, the reaction mixture was poured onto ice (200 g), and concentrated hydrochloric acid was added to acidify the aqueous solution. The resulting solution was extracted with chloroform (150 mL) twice. The chloroform solution was washed with water until the pH was 5 or higher, further washed with saturated brine, and dried over anhydrous magnesium sulfate. The solvent was removed using an evaporator to thereby obtain a yellow liquid. The yellow liquid was purified by silica gel column chromatography to thereby obtain a colorless transparent liquid. A compound represented by the following formula was obtained as a white solid by recrystallization (6.6 g, yield 70%).

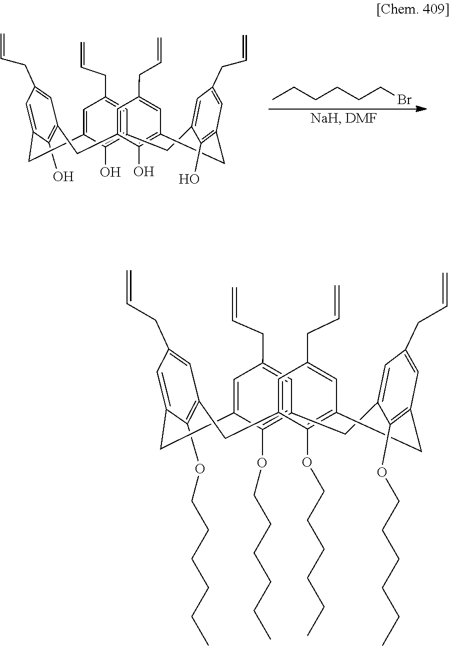

### Synthesis Example 53

The same procedure as in Synthesis Example 52 was repeated except that methyl iodide was used instead of hexyl bromide and that the reaction was performed at room temperature for 24 hours, to thereby obtain a compound represented by the following formula (4.27 g, yield 65%).

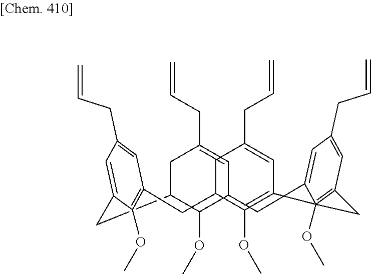

### Synthesis Example 54

The same procedure as in Synthesis Example 52 was repeated except that butyl bromide was used instead of hexyl bromide, to thereby obtain a compound represented by the following formula (6.23 g, yield 75%).

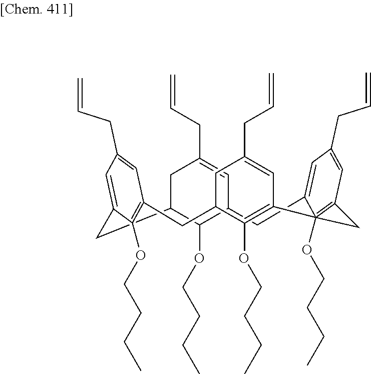

### Synthesis Example 55

The same procedure as in Synthesis Example 52 was repeated except that heptyl bromide was used instead of hexyl bromide, to thereby obtain a compound represented by the following formula (8.02 g, yield 80%).

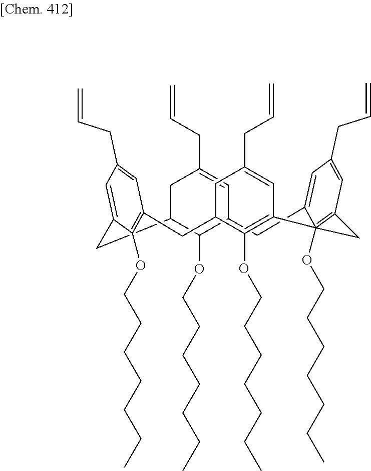

### Synthesis Example 56

The same procedure as in Synthesis Example 52 was repeated except that octadecyl bromide was used instead of hexyl bromide, to thereby obtain a compound represented by the following formula (12.8 g, yield 75%).

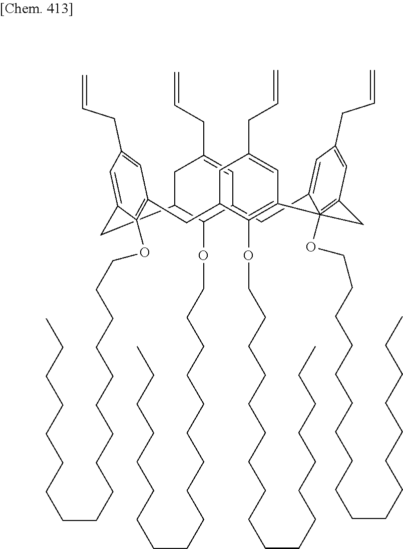

### Synthesis Example 57

Referring to a known document (The Journal of Organic Chemistry, 67, 4722-4733; 2002), a compound represented by the following formula was synthesized using the compound obtained in Synthesis Example 52 (4 g, 4.34 mmol) (yield amount 2.93 g, yield 68%).

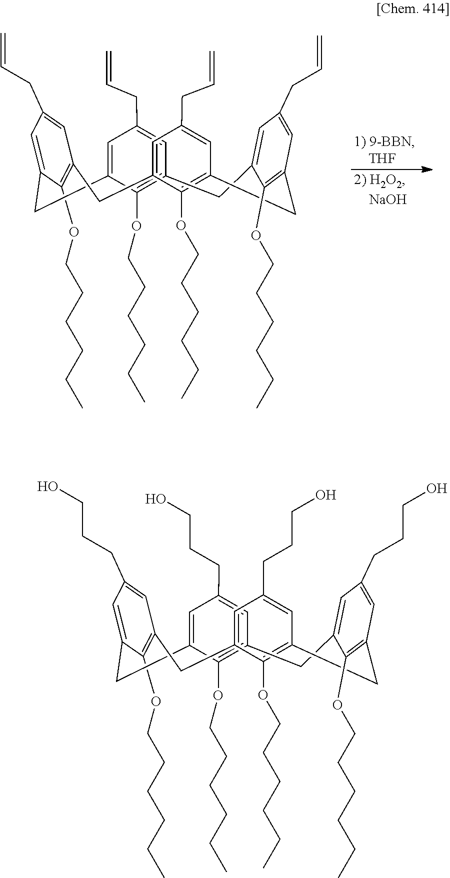 [SMILES: C=CCc1cc2c(OCCCCCC)c(c1)Cc1cc(CC=C)cc(c1OCCCCCC)Cc1cc(CC=C)cc(c1OCCCCCC)Cc1cc(CC=C)cc(c1OCCCCCC)C2 ; CCCCCCOc1c2cc(CCCO)cc1Cc1cc(CCCO)cc(c1OCCCCCC)Cc1cc(CCCO)cc(c1OCCCCCC)Cc1cc(CCCO)cc(c1OCCCCCC)C2]

### Synthesis Example 58

The same procedure as in Synthesis Example 57 was repeated except that the compound obtained in Synthesis Example 53 (4.0 g, 6.24 mmol) was used instead of the compound obtained in Synthesis Example 52, to thereby obtain a compound represented by the following formula (4.5 g, yield 72%).

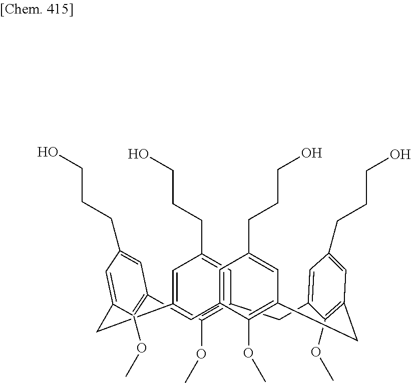 [SMILES: COc1c2cc(CCCO)cc1Cc1cc(CCCO)cc(c1OC)Cc1cc(CCCO)cc(c1OC)Cc1cc(CCCO)cc(c1OC)C2]

### Synthesis Example 59

The same procedure as in Synthesis Example 57 was repeated except that the compound obtained in Synthesis Example 54 (4.0 g, 4.94 mmol) was used instead of the compound obtained in Synthesis Example 52, to thereby obtain a compound represented by the following formula (2.59 g, yield 65%).

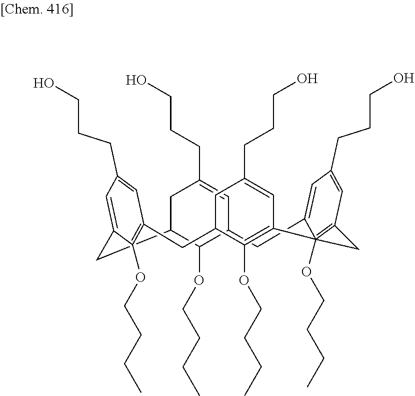

### Synthesis Example 60

The same procedure as in Synthesis Example 57 was repeated except that the compound obtained in Synthesis Example 55 (4.0 g, 4.11 mmol) was used instead of the compound obtained in Synthesis Example 52, to thereby obtain a compound represented by the following formula (3.23 g, yield 75%).

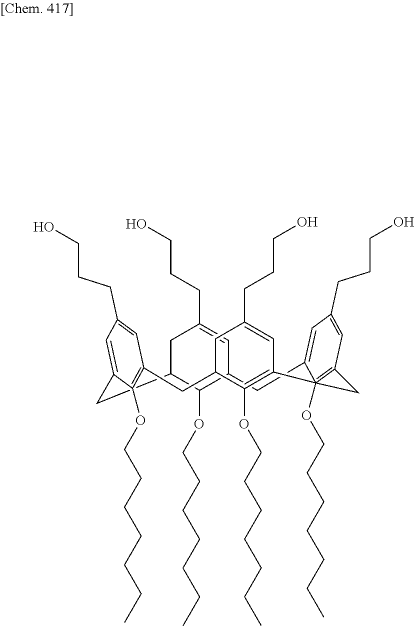

### Synthesis Example 61

The same procedure as in Synthesis Example 57 was repeated except that the compound obtained in Synthesis Example 56 (8.0 g, 5.02 mmol) was used instead of the compound obtained in Synthesis Example 52, to thereby obtain a compound represented by the following formula (5.1 g, yield 61%).

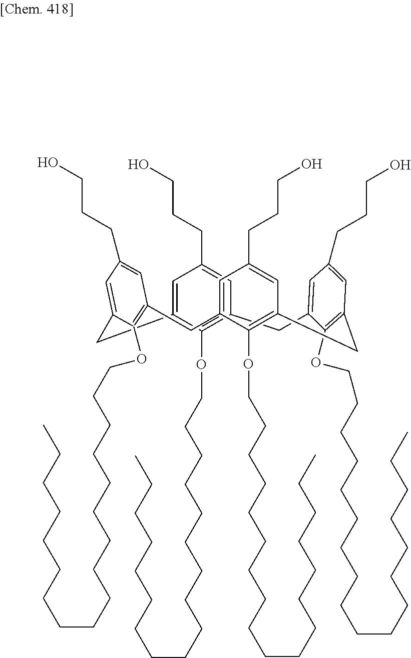 [SMILES: CCCCCCCCCCCCCCCCCCOc1c2cc(CCCO)cc1Cc1cc(CCCO)cc(c1OCCCCCCCCCCCCCCCCCC)Cc1cc(CCCO)cc(c1OCCCCCCCCCCCCCCCCCC)Cc1cc(CCCO)cc(c1OCCCCCCCCCCCCCCCCCC)C2]

### Example 31

A 200 mL four-necked flask equipped with a stirrer, a dropping funnel, and a thermometer was charged, in a nitrogen atmosphere, with the compound obtained in Synthesis Example 47 (3.0 g, 3.94 mmol), 8.268 g (31.52 mmol) of triphenylphosphine, 1.136 g (15.76 mmol) of acrylic acid, 1.609 g (15.76 mmol) of acetoacetic acid, and 68.8 mL of tetrahydrofuran, and the mixture was stirred. Then 6.374 g (31.52 mmol) of diisopropyl azodicarboxylate was added dropwise to the mixture placed in an ice bath over 30 minutes, and the resulting mixture was further stirred at room temperature for 14 hours. The reaction solution was concentrated in an evaporator, and hexane was added to precipitate and remove by-products such as triphenylphosphine. The obtained yellow viscous liquid was purified by silica gel column chromatography to thereby obtain the target compounds 01-6, 02-6, 03-6, and 04-6 as follows. 01-6 (0.538 g, yield 11.5%). A mixture of 02-6 and 03-6 (2.216 g, yield 48.6%). 04-6 (0.586 g, yield 13.2%).

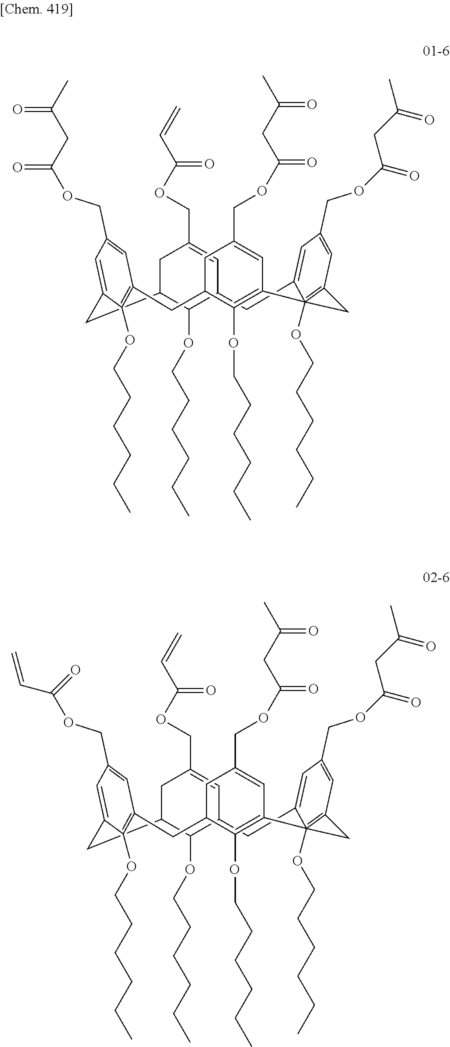

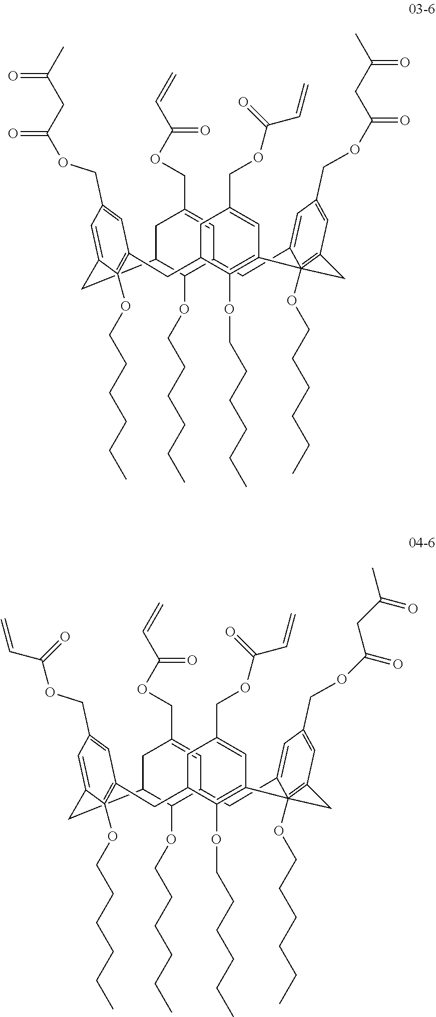

### Example 32

The same procedure as in Example 31 was repeated except that the compound obtained in Synthesis Example 48 (3.0 g, 4.99 mmol) was used instead of the compound obtained in Synthesis Example 47, to thereby obtain the target compounds 01-1, 02-1, 03-1, and 04-1 as follows. 01-1 (0.580 g, yield 12.8%). A mixture of 02-1 and 03-1 (2.159 g, yield 49.3%). 04-1 (0.499 g, yield 11.8%).

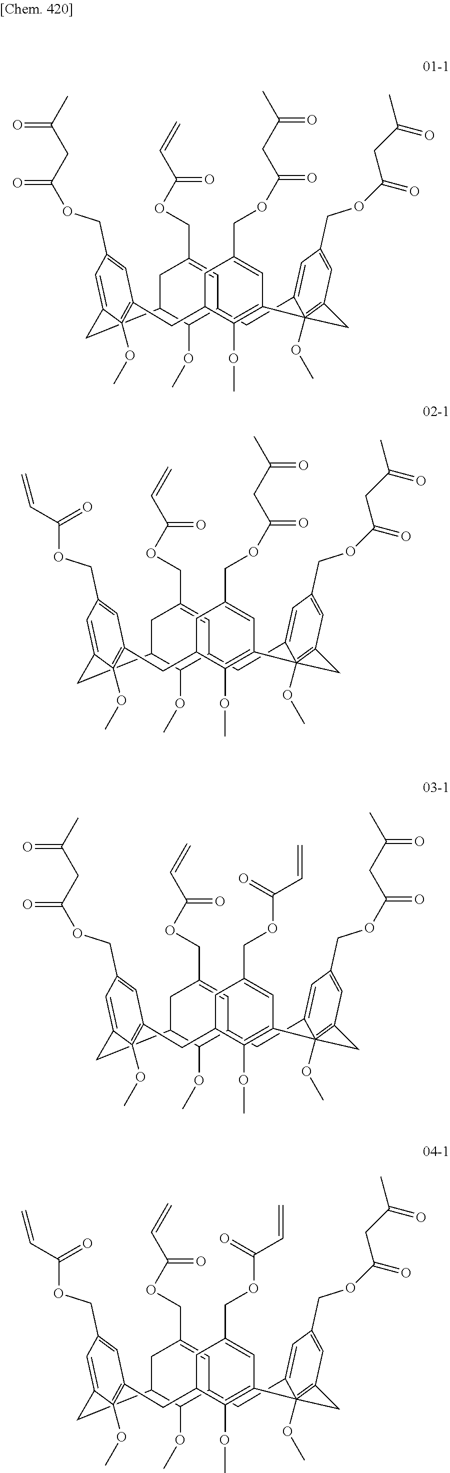

### Example 33

The same procedure as in Example 31 was repeated except that the compound obtained in Synthesis Example 49 (3.0 g, 3.9 mmol) was used instead of the compound obtained in Synthesis Example 47, to thereby obtain the target compounds 01-4, 02-4, 03-4, and 04-4 as follows. 01-4 (0.533 g, yield 12.7%). A mixture of 02-4 and 03-4 (1.941 g, yield 47.6%). 04-4 (0.562 g, yield 14.2%).

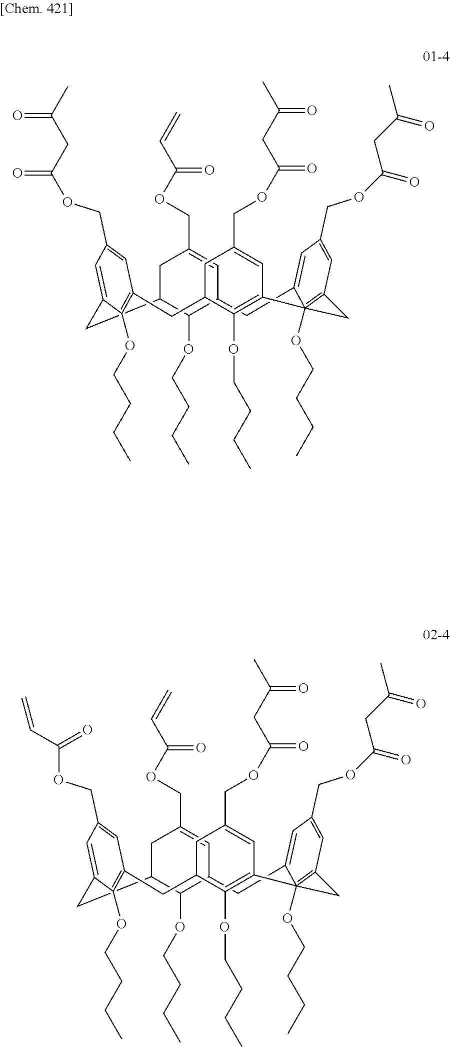

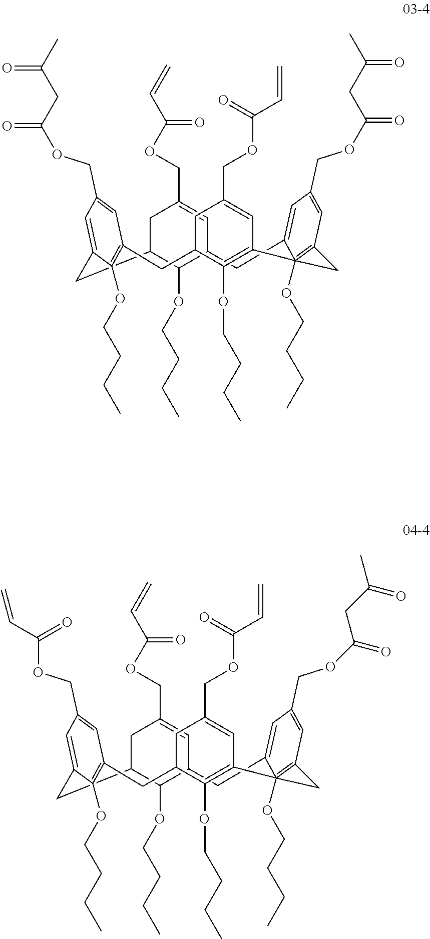

### Example 34

The same procedure as in Example 31 was repeated except that the compound obtained in Synthesis Example 50 (3.0 g, 3.2 mmol) was used instead of the compound obtained in Synthesis Example 47, to thereby obtain the target compounds 01-7, 02-7, 03-7, and 04-7 as follows. 01-7 (0.505 g, yield 12.7%). A mixture of 02-7 and 03-7 (1.946 g, yield 50.1%). 04-7 (0.428 g, yield 11.3%).

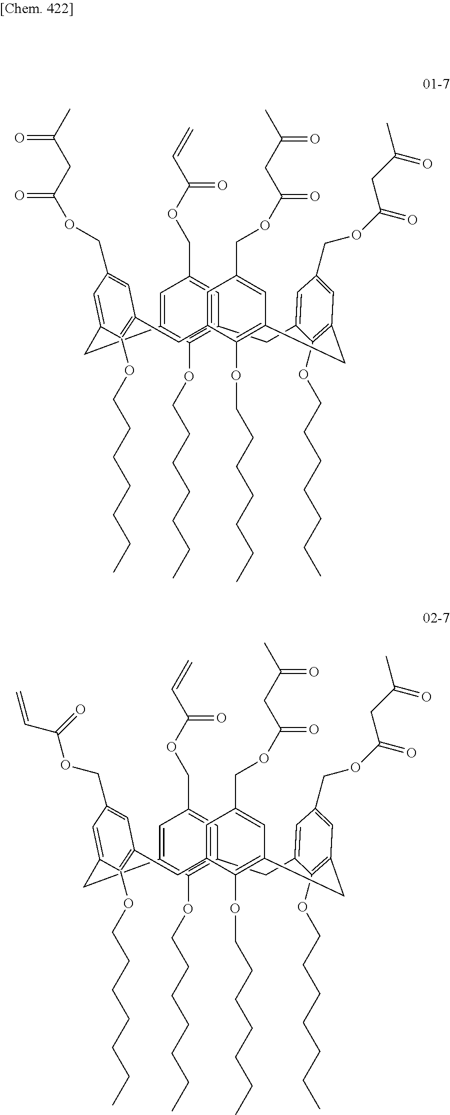 [SMILES: C=CC(=O)OCc1cc2c(OCCCCCCC)c(c1)Cc1cc(COC(=O)CC(C)=O)cc(c1OCCCCCCC)Cc1cc(COC(=O)CC(C)=O)cc(c1OCCCCCCC)Cc1cc(COC(=O)CC(C)=O)cc(c1OCCCCCCC)C2 ; C=CC(=O)OCc1cc2c(OCCCCCCC)c(c1)Cc1cc(COC(=O)CC(C)=O)cc(c1OCCCCCCC)Cc1cc(COC(=O)CC(C)=O)cc(c1OCCCCCCC)Cc1cc(COC(=O)C=C)cc(c1OCCCCCCC)C2]

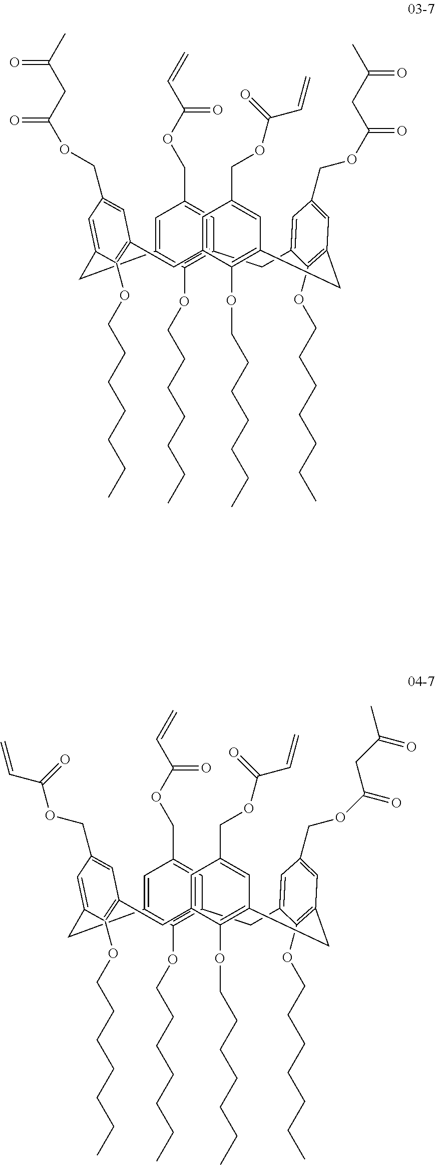 [SMILES: C=CC(=O)OCc1cc2c(OCCCCCCC)c(c1)Cc1cc(COC(=O)CC(C)=O)cc(c1OCCCCCCC)Cc1cc(COC(=O)C=C)cc(c1OCCCCCCC)Cc1cc(COC(=O)CC(C)=O)cc(c1OCCCCCCC)C2 ; C=CC(=O)OCc1cc2c(OCCCCCCC)c(c1)Cc1cc(COC(=O)C=C)cc(c1OCCCCCCC)Cc1cc(COC(=O)CC(C)=O)cc(c1OCCCCCCC)Cc1cc(COC(=O)C=C)cc(c1OCCCCCCC)C2]

### Example 35

The same procedure as in Example 31 was repeated except that the compound obtained in Synthesis Example 51 (3.0 g, 1.93 mmol) was used instead of the compound obtained in Synthesis Example 47, to thereby obtain the target compounds 01-18, 02-18, 03-18, and 04-18 as follows. 01-18 (0.417 g, yield 11.6%). A mixture of 02-18 and 03-18 (1.643 g, yield 46.5%). 04-18 (0.375 g, yield 10.8%).

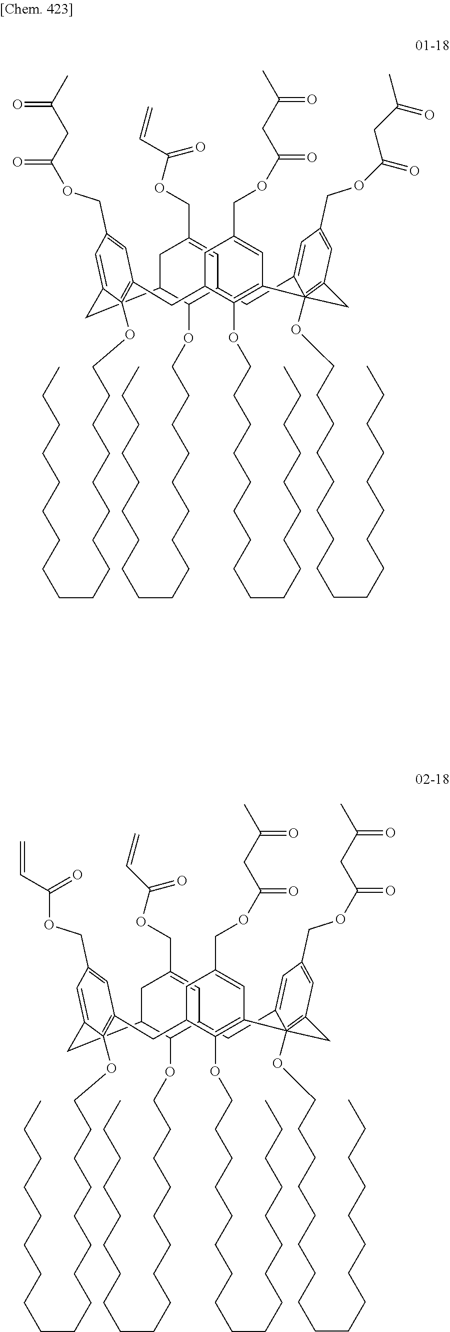

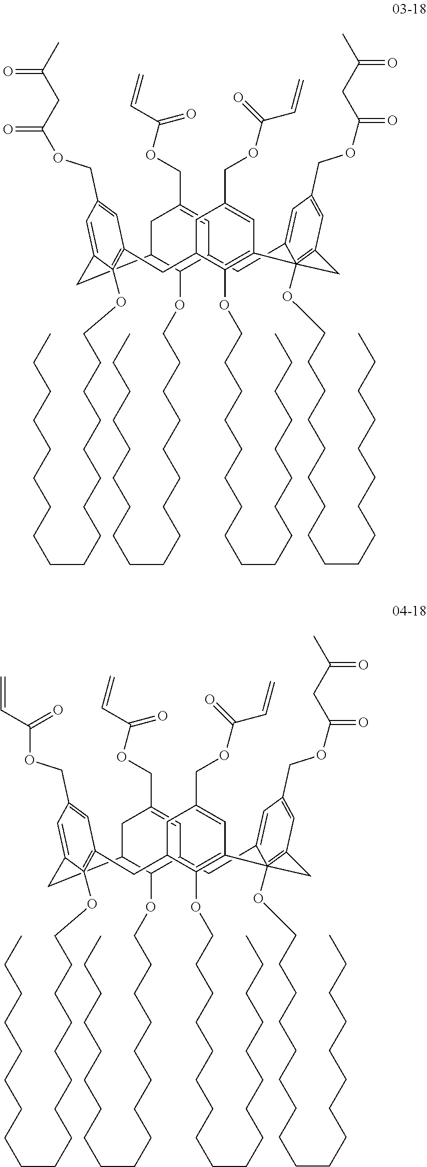

### Synthesis Example 62

A 100 mL four-necked flask equipped with a stirrer, a thermometer, and a reflux condenser tube was charged with 2.00 g (2.27 mmol) of the compound obtained in Synthesis Example 47, 3.57 g (13.62 mmol) of triphenylphosphine, 2.95 g (13.62 mmol) of 2-[[[(1,1-dimethylethyl)dimethylsilyl]oxy]2-propenoic acid, and 38 mL of tetrahydrofuran, and the mixture was stirred. Then 2.75 g (13.62 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 2.85 g, yield 75.0%).

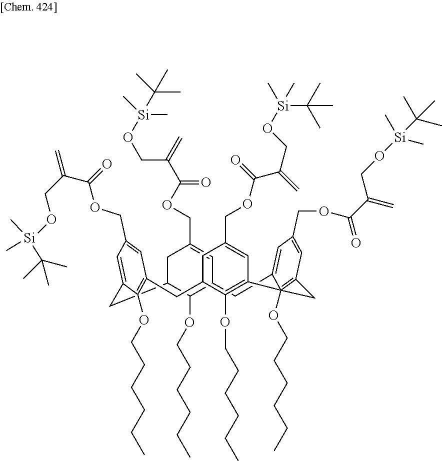

### Synthesis Example 63

The same procedure as in Synthesis Example 62 was repeated except that the compound obtained in Synthesis Example 48 (2.00 g, 3.33 mmol) was used instead of the compound obtained in Synthesis Example 47, to thereby obtain a compound represented by the following formula (3.26 g, yield 70.2%).

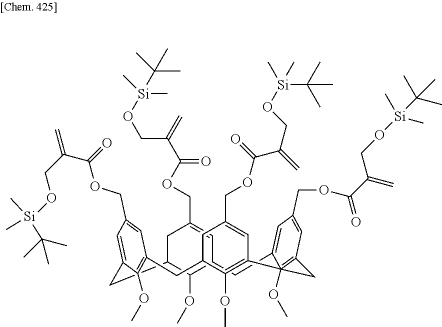

### Synthesis Example 64

The same procedure as in Synthesis Example 62 was repeated except that the compound obtained in Synthesis Example 49 (2.00 g, 2.60 mmol) was used instead of the compound obtained in Synthesis Example 47, to thereby obtain a compound represented by the following formula (3.12 g, yield 76.8%).

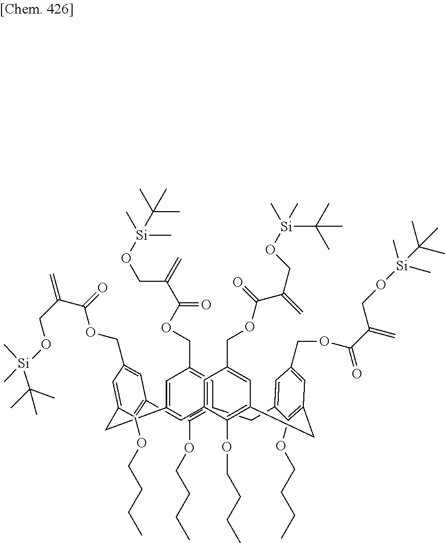 [SMILES: C=C(CO[Si](C)(C)C(C)(C)C)C(=O)OCc1cc2c(OCCCC)c(c1)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)C2]

### Synthesis Example 65

The same procedure as in Synthesis Example 62 was repeated except that the compound obtained in Synthesis Example 50 (2.00 g, 2.13 mmol) was used instead of the compound obtained in Synthesis Example 47, to thereby obtain a compound represented by the following formula (2.74 g, yield 74.2%).

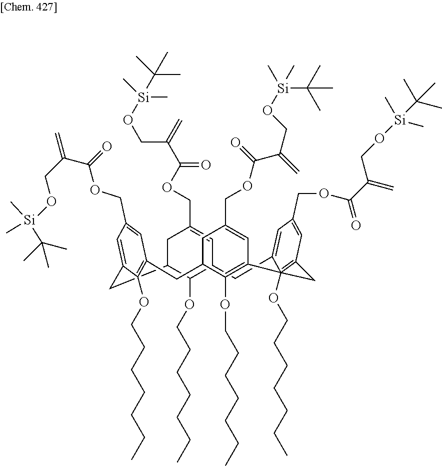

### Synthesis Example 66

The same procedure as in Synthesis Example 62 was repeated except that the compound obtained in Synthesis Example 51 (2.00 g, 1.29 mmol) was used instead of the compound obtained in Synthesis Example 47, to thereby obtain a compound represented by the following formula (2.58 g, yield 85.3%).

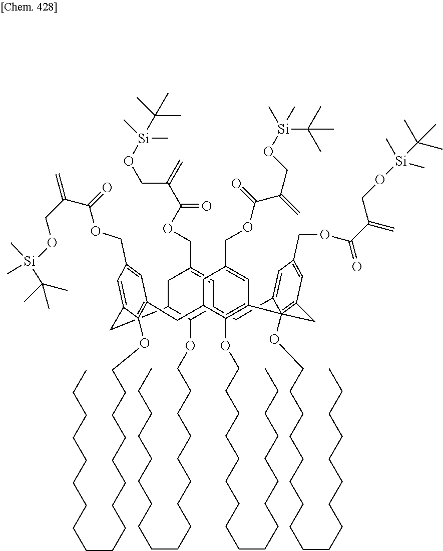

### Synthesis Example 67

A 100 mL four-necked flask equipped with a stirrer, a thermometer, and a reflux condenser tube was charged with 2.50 g (1.49 mmol) of the compound obtained in Synthesis Example 62, 0.538 g (8.96 mmol) of acetic acid, and 60 mL of tetrahydrofuran, and the mixture was stirred. A colorless transparent solution. Then tetrabutylammonium fluoride (an about 1 mol/L tetrahydrofuran solution 8.96 mL (8.96 mmol) was slowly added dropwise to the mixture placed in an ice bath under stirring, and the resulting mixture was further stirred at room temperature for 12 hours. A saturated aqueous ammonium chloride solution was added to the reaction mixture, and 30 mL of chloroform was added. The reaction mixture was transferred to a separatory funnel, and the organic layer was separated. The aqueous layer was extracted with 30 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula as a white solid (yield amount 1.663 g, yield 91.5%).

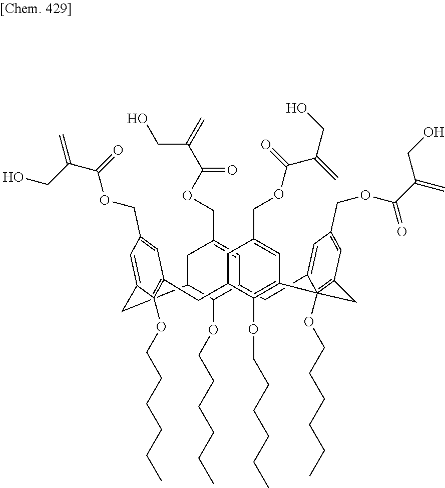

### Synthesis Example 68

The same procedure as in Synthesis Example 67 was repeated except that the compound obtained in Synthesis Example 63 (2.5 g, 1.79 mmol) was used instead of the compound obtained in Synthesis Example 62, to thereby obtain a compound represented by the following formula (1.551 g, yield 92.3%).

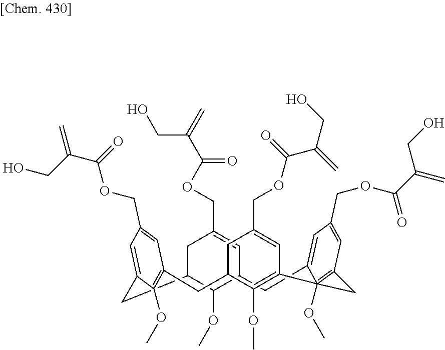

### Synthesis Example 69

The same procedure as in Synthesis Example 67 was repeated except that the compound obtained in Synthesis Example 64 (2.5 g, 1.60 mmol) was used instead of the compound obtained in Synthesis Example 62, to thereby obtain a compound represented by the following formula (1.671 g, yield 94.5%).

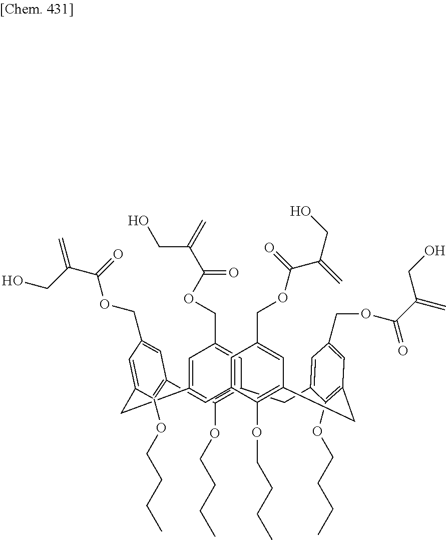 [SMILES: C=C(CO)C(=O)OCc1cc2c(OCCCC)c(c1)Cc1cc(COC(=O)C(=C)CO)cc(c1OCCCC)Cc1cc(COC(=O)C(=C)CO)cc(c1OCCCC)Cc1cc(COC(=O)C(=C)CO)cc(c1OCCCC)C2]

### Synthesis Example 70

The same procedure as in Synthesis Example 67 was repeated except that the compound obtained in Synthesis Example 65 (2.5 g, 1.44 mmol) was used instead of the compound obtained in Synthesis Example 62, to thereby obtain a compound represented by the following formula (1.759 g, yield 95.6%).

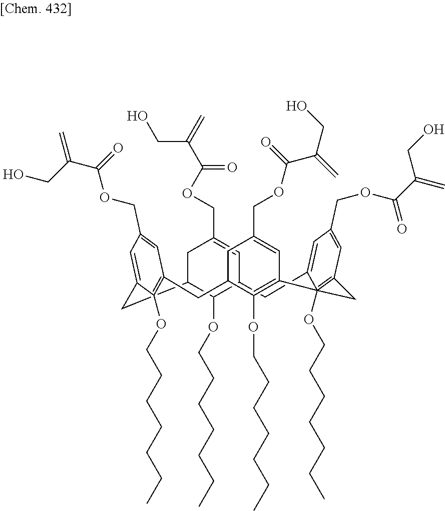

### Synthesis Example 71

The same procedure as in Synthesis Example 67 was repeated except that the compound obtained in Synthesis Example 66 (2.50 g, 1.06 mmol) was used instead of the compound obtained in Synthesis Example 62, to thereby obtain a compound represented by the following formula (1.90 g, yield 94.8%).

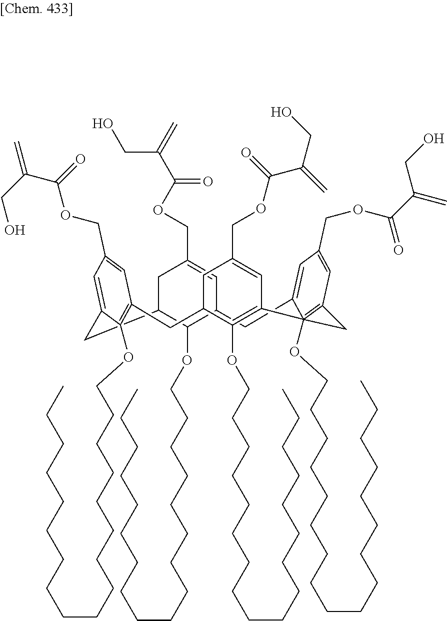

### Example 36

A 100 mL four-necked flask equipped with a stirrer, a dropping funnel, and a thermometer was charged, in a nitrogen atmosphere, with the compound obtained in Synthesis Example 67 (1.5 g, 1.23 mmol), 2.585 g (9.86 mmol) of triphenylphosphine, 1.006 g (9.86 mmol) of acetoacetic acid, and 24 mL of tetrahydrofuran, and the mixture was stirred. Then 1.993 g (9.86 mmol) of diisopropyl azodicarboxylate was added dropwise to the mixture placed in an ice bath over 30 minutes, and the resulting mixture was further stirred at room temperature for 14 hours. The reaction solution was concentrated in an evaporator, and hexane was added to precipitate and remove by-products such as triphenylphosphine. The obtained yellow liquid was purified by silica gel column chromatography to thereby obtain the target compound 05-6 (yield amount 1.422 g, yield 74.3%).

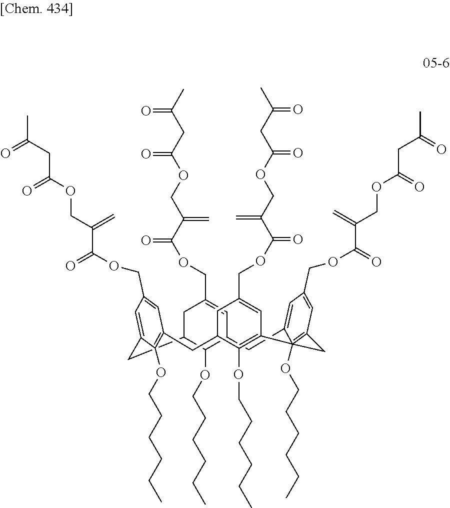

### Example 37

The same procedure as in Example 36 was repeated except that the compound obtained in Synthesis Example 68 (1.50 g, 1.60 mmol) was used instead of the compound obtained in Synthesis Example 67, to thereby obtain the target compound 05-1 (1.457 g, yield 71.5%).

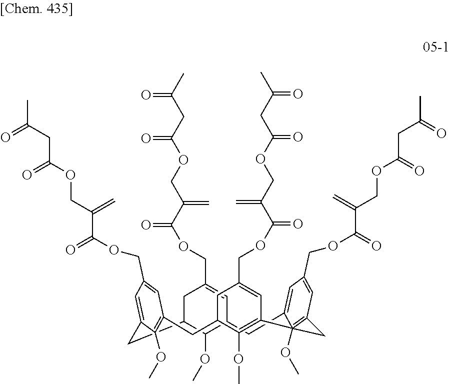

### Example 38

The same procedure as in Example 36 was repeated except that the compound obtained in Synthesis Example 69 (1.50 g, 1.36 mmol) was used instead of the compound obtained in Synthesis Example 67, to thereby obtain the target compound 05-4 (1.438 g, yield 73.5%).

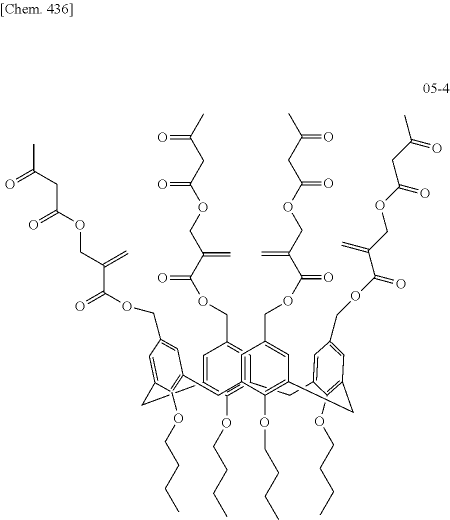 [SMILES: C=C(COC(=O)CC(C)=O)C(=O)OCc1cc2c(OCCCC)c(c1)Cc1cc(COC(=O)C(=C)COC(=O)CC(C)=O)cc(c1OCCCC)Cc1cc(COC(=O)C(=C)COC(=O)CC(C)=O)cc(c1OCCCC)Cc1cc(COC(=O)C(=C)COC(=O)CC(C)=O)cc(c1OCCCC)C2]

### Example 39

The same procedure as in Example 36 was repeated except that the compound obtained in Synthesis Example 70 (1.50 g, 1.18 mmol) was used instead of the compound obtained in Synthesis Example 67, to thereby obtain the target compound 05-7 (1.380 g, yield 72.8%).

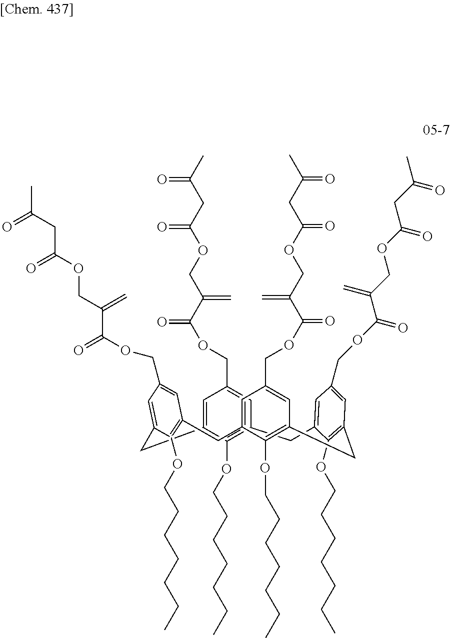 [SMILES: C=C(COC(=O)CC(C)=O)C(=O)OCc1cc2c(OCCCCCCC)c(c1)Cc1cc(COC(=O)C(=C)COC(=O)CC(C)=O)cc(c1OCCCCCCC)Cc1cc(COC(=O)C(=C)COC(=O)CC(C)=O)cc(c1OCCCCCCC)Cc1cc(COC(=O)C(=C)COC(=O)CC(C)=O)cc(c1OCCCCCCC)C2]

### Example 40

The same procedure as in Example 36 was repeated except that the compound obtained in Synthesis Example 71 (1.5 g, 0.79 mmol) was used instead of the compound obtained in Synthesis Example 67, to thereby obtain the target compound 05-18 (1.253 g, yield 70.9%).

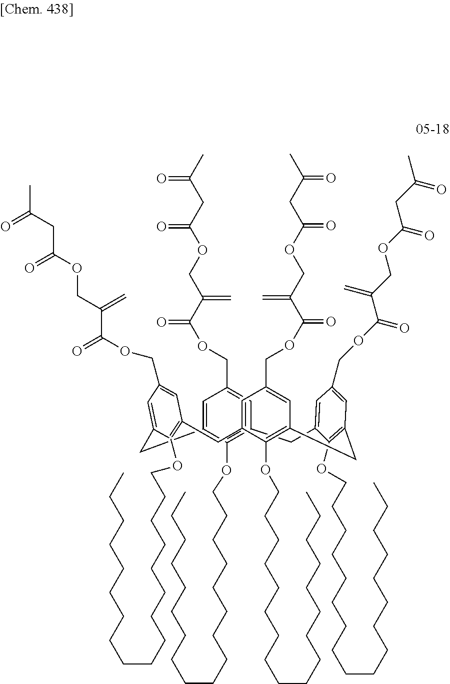 [SMILES: C=C(COC(=O)CC(C)=O)C(=O)OCc1cc2c(OCCCCCCCCCCCCCCCCCC)c(c1)Cc1cc(COC(=O)C(=C)COC(=O)CC(C)=O)cc(c1OCCCCCCCCCCCCCCCCCC)Cc1cc(COC(=O)C(=C)COC(=O)CC(C)=O)cc(c1OCCCCCCCCCCCCCCCCCC)Cc1cc(COC(=O)C(=C)COC(=O)CC(C)=O)cc(c1OCCCCCCCCCCCCCCCCCC)C2]

### Example 41

A 200 mL four-necked flask equipped with a stirrer, a dropping funnel, and a thermometer was charged, in a nitrogen atmosphere, with the compound obtained in Synthesis Example 57 (3.0 g, 3.02 mmol), 6.336 g (24.16 mmol) of triphenylphosphine, 0.870 g (12.08 mmol) of acrylic acid, 1.233 g (12.08 mmol) of acetoacetic acid, and 55 mL of tetrahydrofuran, and the mixture was stirred. Then 4.885 g (24.16 mmol) of diisopropyl azodicarboxylate was added dropwise to the mixture placed in an ice bath over 30 minutes, and the resulting mixture was further stirred at room temperature for 14 hours. The reaction solution was concentrated in an evaporator, and hexane was added to precipitate and remove by-products such as triphenylphosphine. The obtained yellow viscous liquid was purified by silica gel column chromatography to thereby obtain the target compounds 06-6, 07-6, 08-6, and 09-6 as follows. 06-6 (0.424 g, yield 10.8%). A mixture of 07-6 and 08-6 (1.821 g, yield 47.5%). 09-6 (0.554 g, yield 14.8%).

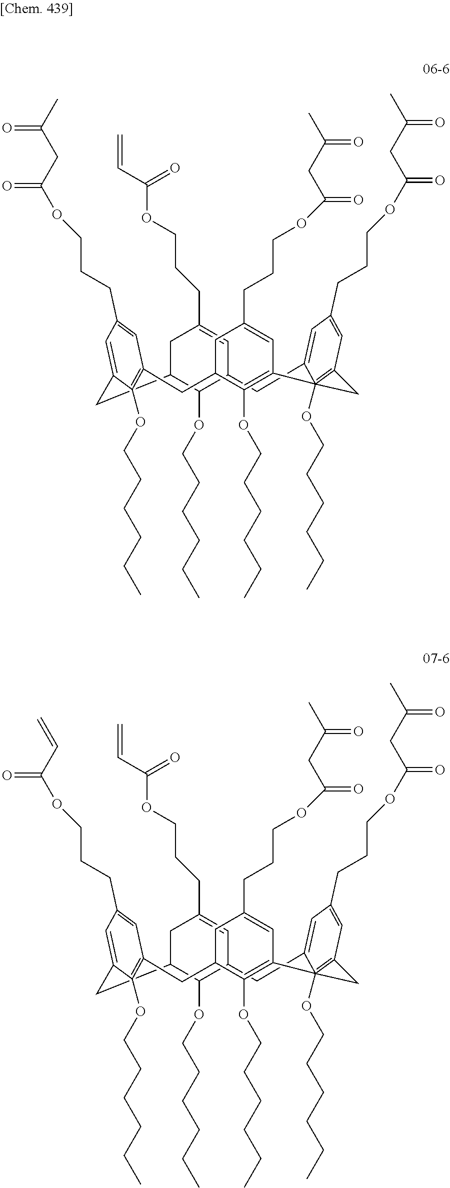

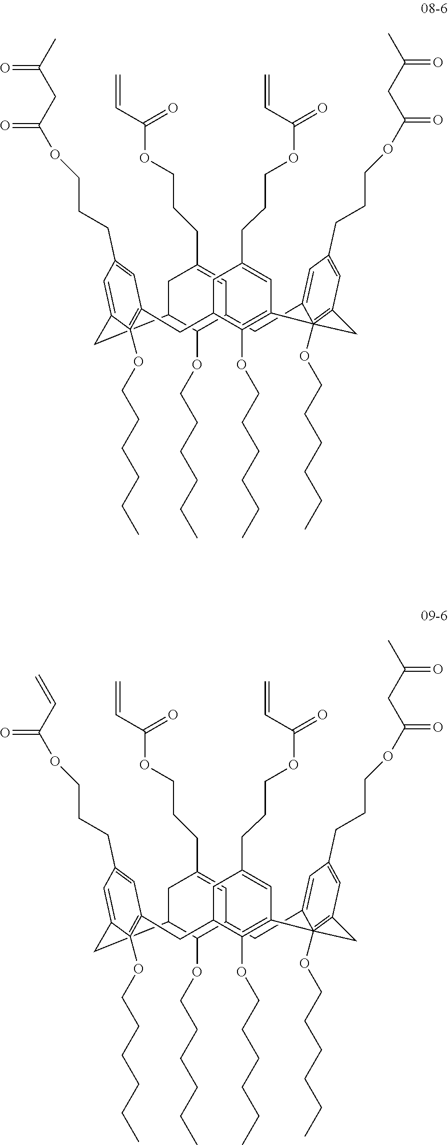

### Example 42

The same procedure as in Example 41 was repeated except that the compound obtained in Synthesis Example 58 (3.00 g, 4.21 mmol) was used instead of the compound obtained in Synthesis Example 57, to thereby obtain the target compounds 06-1, 07-1, 08-1, and 09-1 as follows. 06-1 (0.480 g, yield 11.2%). A mixture of 07-1 and 08-1 (2.027 g, yield 48.7%). 09-1 (0.521 g, yield 12.9%).

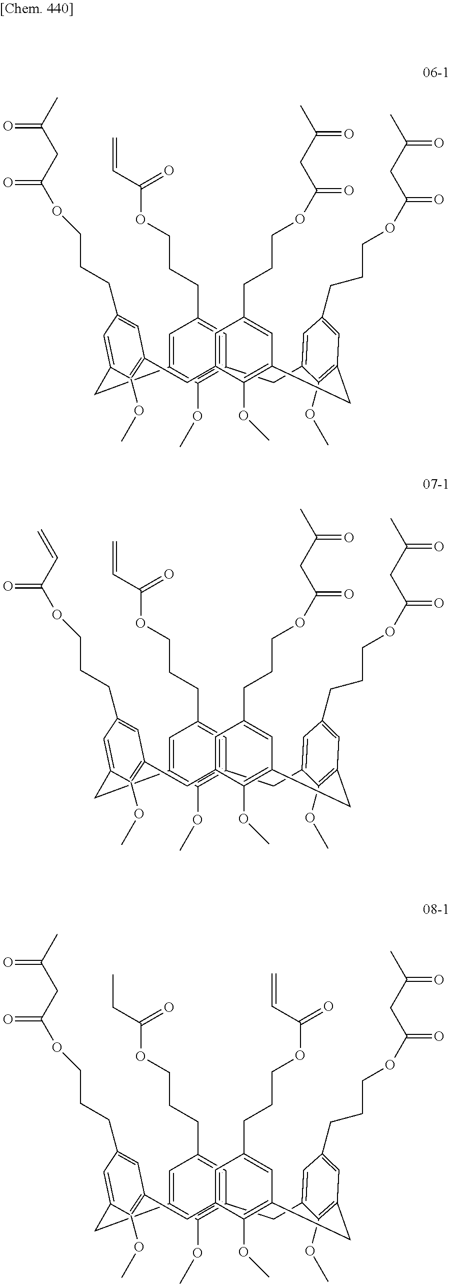 [SMILES: C=CC(=O)OCCCc1cc2c(OC)c(c1)Cc1cc(CCCOC(=O)CC(C)=O)cc(c1OC)Cc1cc(CCCOC(=O)CC(C)=O)cc(c1OC)Cc1cc(CCCOC(=O)CC(C)=O)cc(c1OC)C2 ; C=CC(=O)OCCCc1cc2c(OC)c(c1)Cc1cc(CCCOC(=O)CC(C)=O)cc(c1OC)Cc1cc(CCCOC(=O)CC(C)=O)cc(c1OC)Cc1cc(CCCOC(=O)C=C)cc(c1OC)C2 ; C=CC(=O)OCCCc1cc2c(OC)c(c1)Cc1cc(CCCOC(=O)CC(C)=O)cc(c1OC)Cc1cc(CCCOC(=O)CC)cc(c1OC)Cc1cc(CCCOC(=O)CC(C)=O)cc(c1OC)C2]

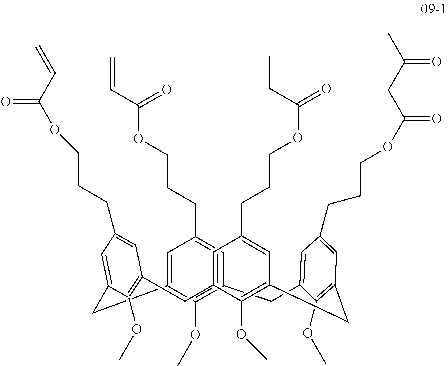 [SMILES: C=CC(=O)OCCCc1cc2c(OC)c(c1)Cc1cc(CCCOC(=O)CC)cc(c1OC)Cc1cc(CCCOC(=O)CC(C)=O)cc(c1OC)Cc1cc(CCCOC(=O)C=C)cc(c1OC)C2]

### Example 43

The same procedure as in Example 41 was repeated except that the compound obtained in Synthesis Example 59 (3.00 g, 3.40 mmol) was used instead of the compound obtained in Synthesis Example 57, to thereby obtain the target compounds 06-4, 07-4, 08-4, and 09-4 as follows. 06-4 (0.416 g, yield 10.3%). A mixture of 07-1 and 08-1 (1.943 g, yield 49.3%). 09-4 (0.480 g, yield 12.5%).

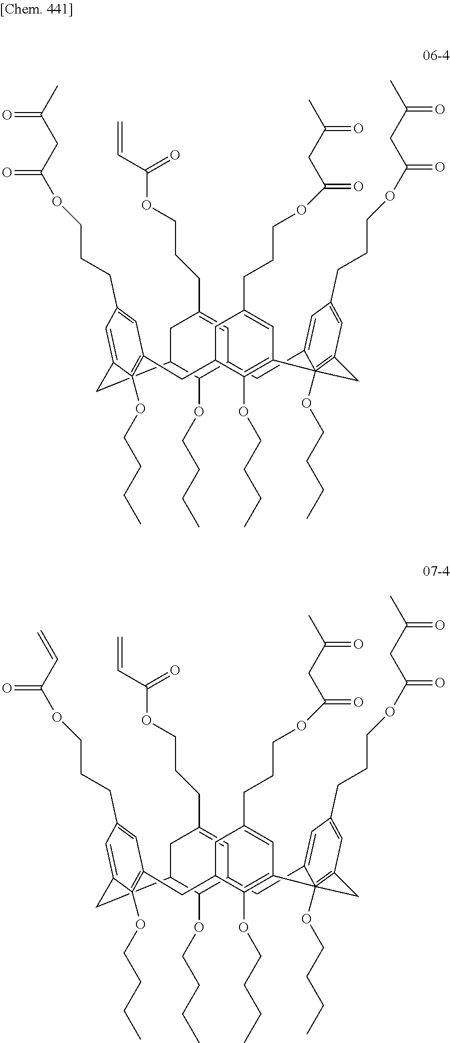

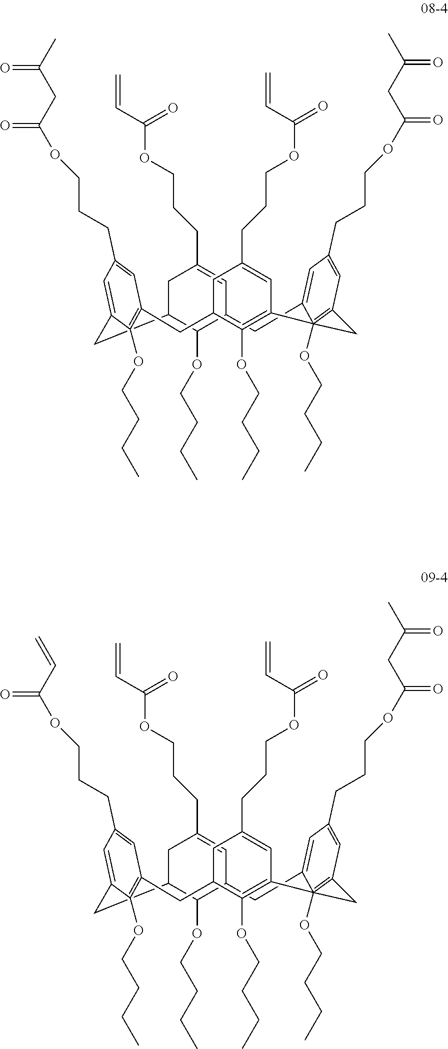

### Example 44

The same procedure as in Example 41 was repeated except that the compound obtained in Synthesis Example 60 (3.00 g, 2.86 mmol) was used instead of the compound obtained in Synthesis Example 57, to thereby obtain the target compounds 06-7, 07-7, 08-7, and 09-7 as follows. 06-7 (0.453 g, yield 11.7%). A mixture of 07-7 and 08-7 (1.918 g, yield 50.6%). 09-7 (0.463 g, yield 12.5%).

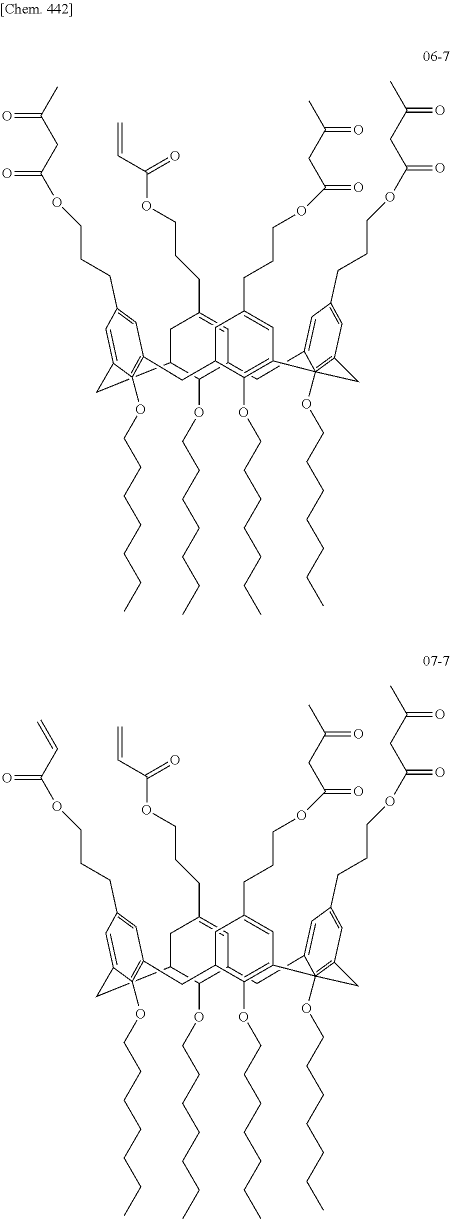

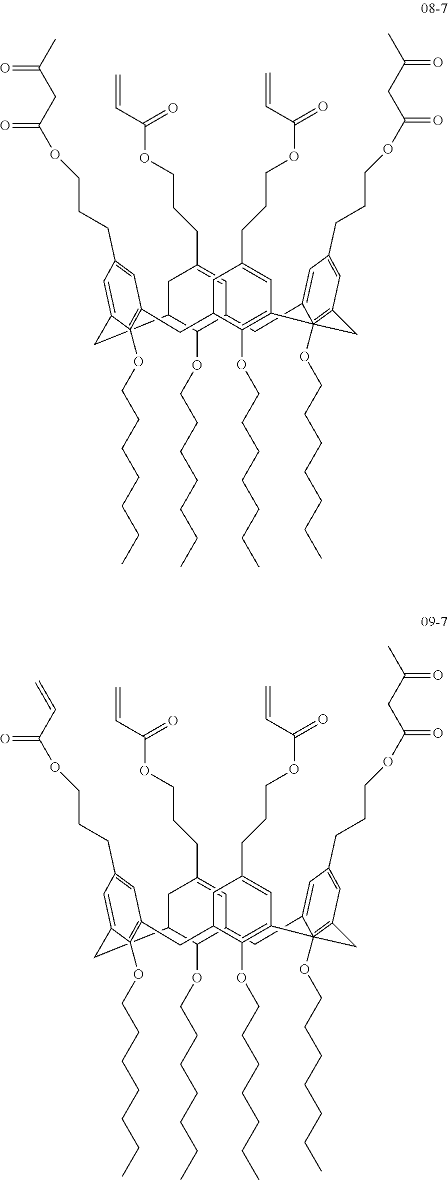

### Example 45

The same procedure as in Example 41 was repeated except that the compound obtained in Synthesis Example 61 (3.00 g, 1.80 mmol) was used instead of the compound obtained in Synthesis Example 57, to thereby obtain the target compounds 06-18, 07-18, 08-18, and 09-18 as follows. 06-18 (0.338 g, yield 9.8%). A mixture of 07-18 and 08-18 (1.603 g, yield 47.2%). 09-18 (0.404 g, yield 12.1%).

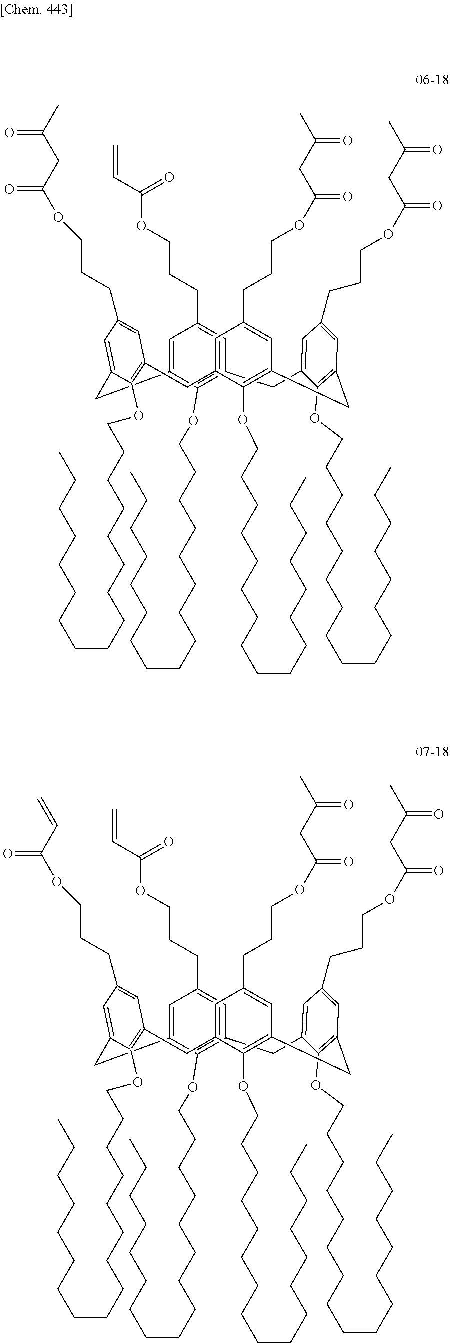 [SMILES: C=CC(=O)OCCCc1cc2c(OCCCCCCCCCCCCCCCCC)c(c1)Cc1cc(CCCOC(=O)CC(C)=O)cc(c1OCCCCCCCCCCCCCCCCC)Cc1cc(CCCOC(=O)CC(C)=O)cc(c1OCCCCCCCCCCCCCCCCC)Cc1cc(CCCOC(=O)CC(C)=O)cc(c1OCCCCCCCCCCCCCCCCC)C2 ; C=CC(=O)OCCCc1cc2c(OCCCCCCCCCCCCCCCCC)c(c1)Cc1cc(CCCOC(=O)CC(C)=O)cc(c1OCCCCCCCCCCCCCCCCC)Cc1cc(CCCOC(=O)CC(C)=O)cc(c1OCCCCCCCCCCCCCCCCC)Cc1cc(CCCOC(=O)C=C)cc(c1OCCCCCCCCCCCCCCCCC)C2]

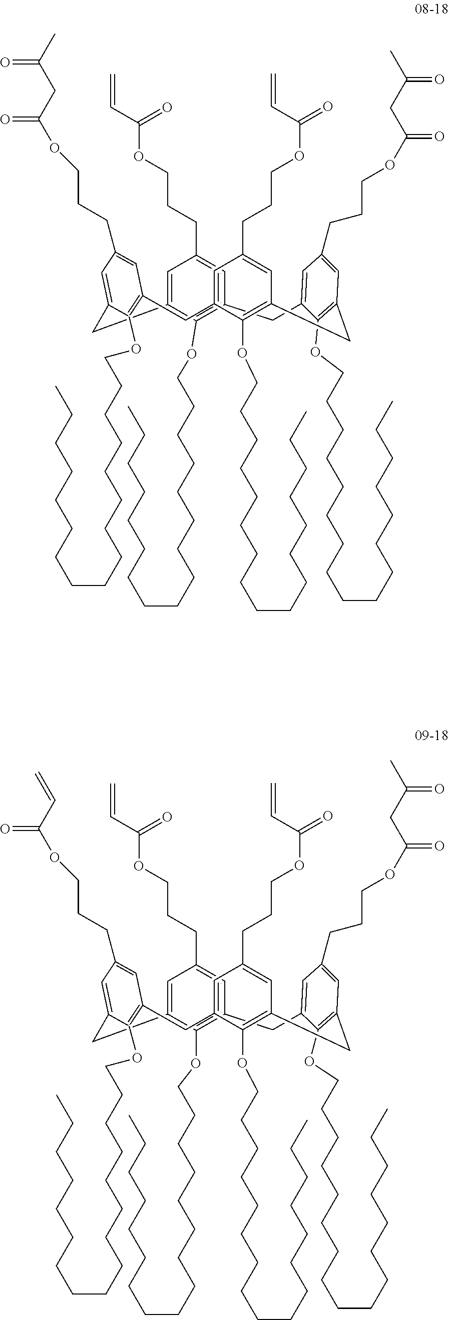 [SMILES: C=CC(=O)OCCCc1cc2c(OCCCCCCCCCCCCCCCCC)c(c1)Cc1cc(CCCOC(=O)CC(C)=O)cc(c1OCCCCCCCCCCCCCCCCC)Cc1cc(CCCOC(=O)C=C)cc(c1OCCCCCCCCCCCCCCCCC)Cc1cc(CCCOC(=O)CC(C)=O)cc(c1OCCCCCCCCCCCCCCCCC)C2 ; C=CC(=O)OCCCc1cc2c(OCCCCCCCCCCCCCCCCC)c(c1)Cc1cc(CCCOC(=O)C=C)cc(c1OCCCCCCCCCCCCCCCCC)Cc1cc(CCCOC(=O)CC(C)=O)cc(c1OCCCCCCCCCCCCCCCCC)Cc1cc(CCCOC(=O)C=C)cc(c1OCCCCCCCCCCCCCCCCC)C2]

### Synthesis Example 72

A 100 mL four-necked flask equipped with a stirrer, a thermometer, and a reflux condenser tube was charged with 2.50 g (2.52 mmol) of the compound obtained in Synthesis Example 57, 3.96 g (15.10 mmol) of triphenylphosphine, 3.267 g (15.10 mmol) of 2-[[[(1,1-dimethylethyl)dimethylsilyl]oxy]2-propenoic acid, and 43 mL of tetrahydrofuran, and the mixture was stirred. Then 3.053 g (15.10 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 3.251 g, yield 72.3%).

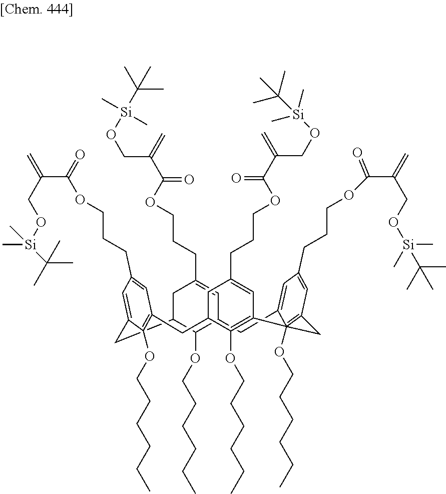

### Synthesis Example 73

The same procedure as in Synthesis Example 72 was repeated except that the compound obtained in Synthesis Example 58 (2.50 g, 3.33 mmol) was used instead of the compound obtained in Synthesis Example 57, to thereby obtain a compound represented by the following formula (3.782 g, yield 71.6%).

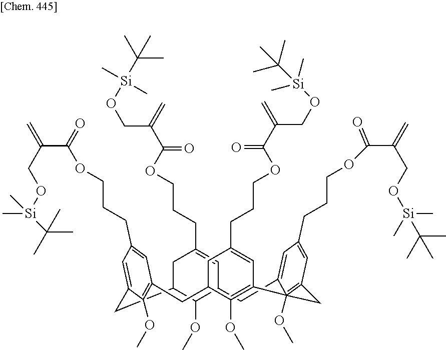

### Synthesis Example 74

The same procedure as in Synthesis Example 72 was repeated except that the compound obtained in Synthesis Example 59 (2.50 g, 2.84 mmol) was used instead of the compound obtained in Synthesis Example 57, to thereby obtain a compound represented by the following formula (3.553 g, yield 74.8%).

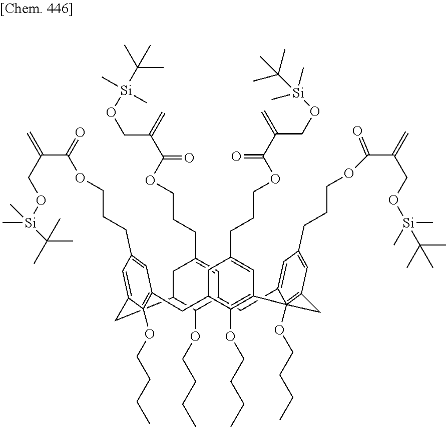

### Synthesis Example 75

The same procedure as in Synthesis Example 72 was repeated except that the compound obtained in Synthesis Example 60 (2.50 g, 2.38 mmol) was used instead of the compound obtained in Synthesis Example 57, to thereby obtain a compound represented by the following formula (3.305 g, yield 75.3%).

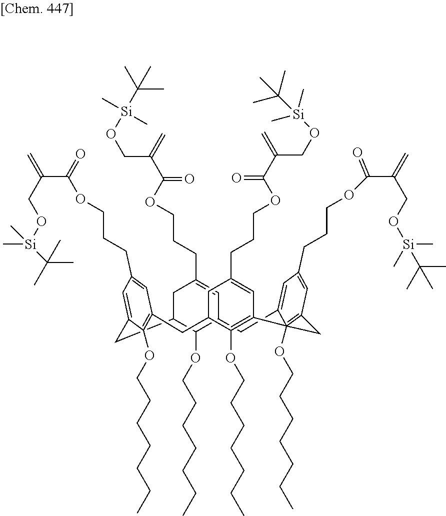

### Synthesis Example 76

The same procedure as in Synthesis Example 72 was repeated except that the compound obtained in Synthesis Example 61 (2.50 g, 1.50 mmol) was used instead of the compound obtained in Synthesis Example 57, to thereby obtain a compound represented by the following formula (3.011 g, yield 81.6%).

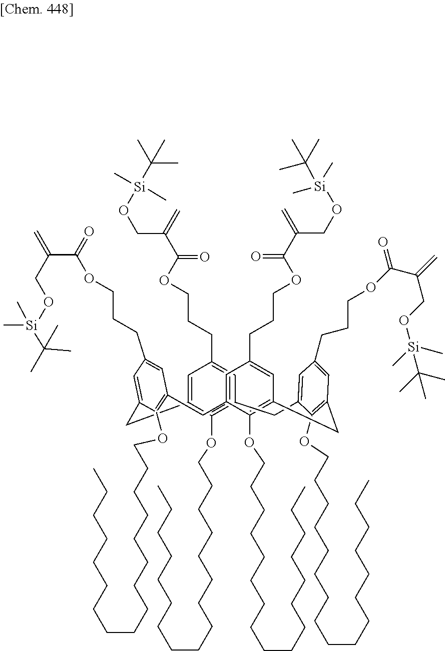 [SMILES: C=C(CO[Si](C)(C)C(C)(C)C)C(=O)OCCCc1cc2c(OCCCCCCCCCCCCCCCCC)c(c1)Cc1cc(CCCOC(=O)C(=C)CO[Si](C)(C)C(C)(C)C)cc(c1OCCCCCCCCCCCCCCCCC)Cc1cc(CCCOC(=O)C(=C)CO[Si](C)(C)C(C)(C)C)cc(c1OCCCCCCCCCCCCCCCCC)Cc1cc(CCCOC(=O)C(=C)CO[Si](C)(C)C(C)(C)C)cc(c1OCCCCCCCCCCCCCCCCC)C2]

### Synthesis Example 77

A 200 mL four-necked flask equipped with a stirrer, a thermometer, and a reflux condenser tube was charged with 3.50 g (1.96 mmol) of the compound obtained in Synthesis Example 72, 0.706 g (11.75 mmol) of acetic acid, and 78.4 mL of tetrahydrofuran, and the mixture was stirred. A colorless transparent solution. Then tetrabutylammonium fluoride (an about 1 mol/L tetrahydrofuran solution 11.75 mL (11.75 mmol) was slowly added dropwise to the mixture placed in an ice bath under stirring. The resulting mixture was stirred at room temperature for 12 hours. A saturated aqueous ammonium chloride solution was added to the reaction mixture, and then 50 mL of chloroform was added. The reaction mixture was transferred to a separatory funnel, and the organic layer was separated. Then the aqueous layer was extracted with 50 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula (yield amount 2.417 g, yield 92.8%).

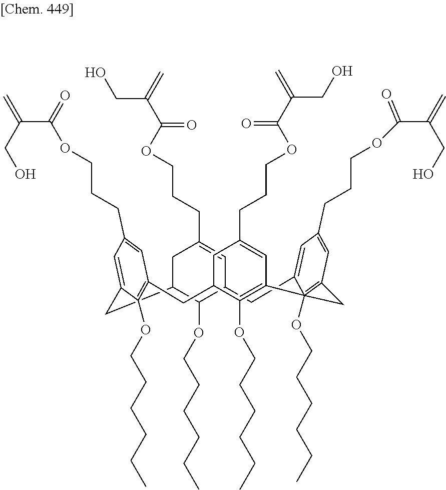

### Synthesis Example 78

The same procedure as in Synthesis Example 77 was repeated except that the compound obtained in Synthesis Example 73 (3.50 g, 2.32 mmol) was used instead of the compound obtained in Synthesis Example 72, to thereby obtain a compound represented by the following formula (2.214 g, yield 90.8%).

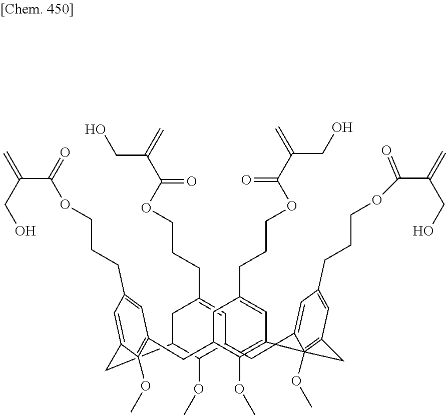

### Synthesis Example 79

The same procedure as in Synthesis Example 77 was repeated except that the compound obtained in Synthesis Example 74 (3.50 g, 2.32 mmol) was used instead of the compound obtained in Synthesis Example 72, to thereby obtain a compound represented by the following formula (2.344 g, yield 92.1%).

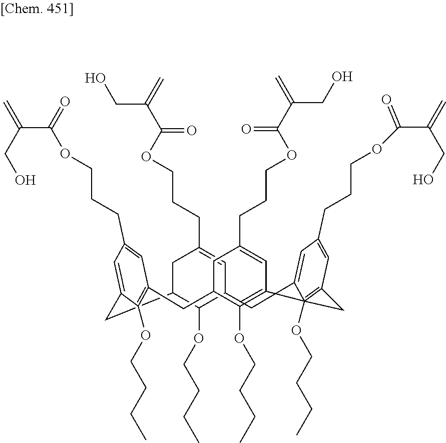

### Synthesis Example 80

The same procedure as in Synthesis Example 77 was repeated except that the compound obtained in Synthesis Example 75 (3.50 g, 2.32 mmol) was used instead of the compound obtained in Synthesis Example 72, to thereby obtain a compound represented by the following formula (2.466 g, yield 93.7%).

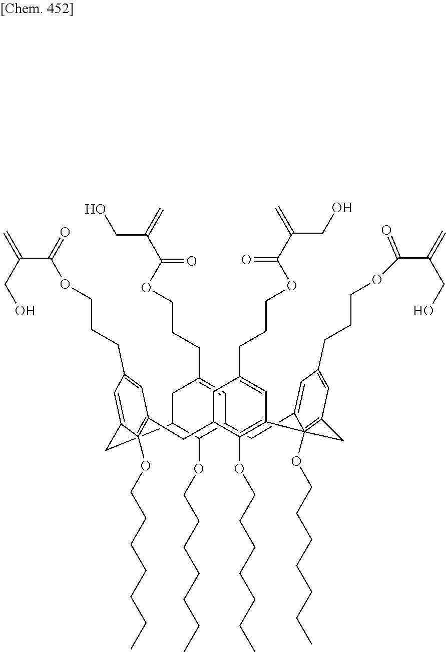

### Synthesis Example 81

The same procedure as in Synthesis Example 77 was repeated except that the compound obtained in Synthesis Example 76 (3.50 g, 1.42 mmol) was used instead of the compound obtained in Synthesis Example 72, to thereby obtain a compound represented by the following formula (2.608 g, yield 91.5%).

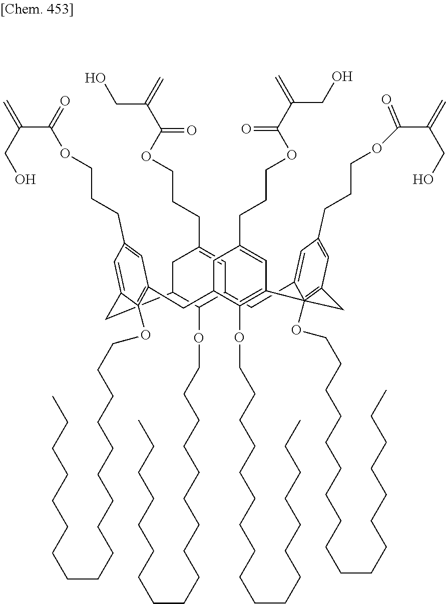

### Example 46

A 100 mL four-necked flask equipped with a stirrer, a dropping funnel, and a thermometer was charged, in a nitrogen atmosphere, with the compound obtained in Synthesis Example 77 (2.0 g, 1.50 mmol), 3.156 g (12.03 mmol) of triphenylphosphine, 1.228 g (12.03 mmol) of acetoacetic acid, and 30 mL of tetrahydrofuran, and the mixture was stirred. Then 2.433 g (12.03 mmol) of diisopropyl azodicarboxylate was added dropwise to the mixture placed in an ice bath over 30 minutes, and the resulting mixture was further stirred at room temperature for 14 hours. The reaction solution was concentrated in an evaporator, and hexane was added to precipitate and remove by-products such as triphenylphosphine. The obtained yellow liquid was purified by silica gel column chromatography to thereby obtain the target compound 010-6 (yield amount 1.812 g, yield 72.3%).

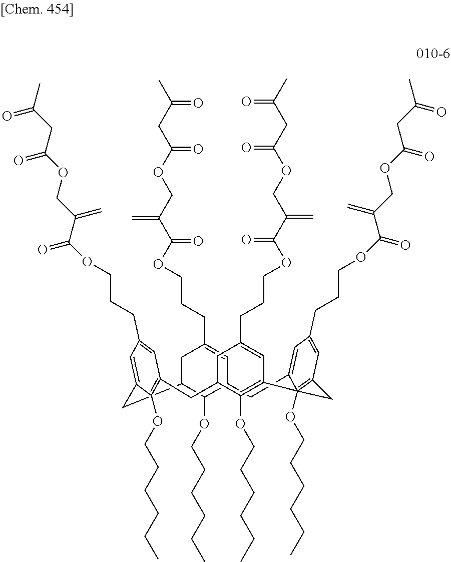

### Example 47

The same procedure as in Example 46 was repeated except that the compound obtained in Synthesis Example 78 (2.00 g, 1.91 mmol) was used instead of the compound obtained in Synthesis Example 77, to thereby obtain the target compound 010-1 (1.888 g, yield 71.5%).

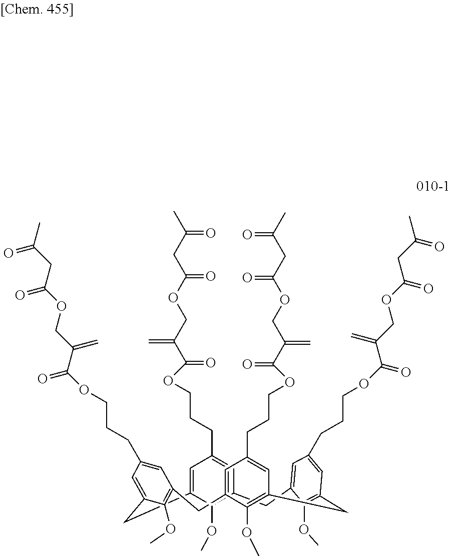 [SMILES: C=C(COC(=O)CC(C)=O)C(=O)OCCCc1cc2c(OC)c(c1)Cc1cc(CCCOC(=O)C(=C)COC(=O)CC(C)=O)cc(c1OC)Cc1cc(CCCOC(=O)C(=C)COC(=O)CC(C)=O)cc(c1OC)Cc1cc(CCCOC(=O)C(=C)COC(=O)CC(C)=O)cc(c1OC)C2]

### Example 48

The same procedure as in Example 46 was repeated except that the compound obtained in Synthesis Example 79 (2.00 g, 1.64 mmol) was used instead of the compound obtained in Synthesis Example 77, to thereby obtain the target compound 010-4 (1.959 g, yield 73.7%).

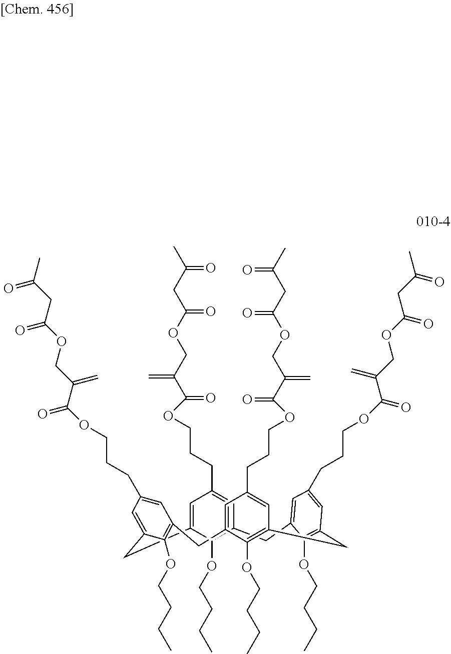 [SMILES: C=C(COC(=O)CC(C)=O)C(=O)OCCCc1cc2c(OCCCC)c(c1)Cc1cc(CCCOC(=O)C(=C)COC(=O)CC(C)=O)cc(c1OCCCC)Cc1cc(CCCOC(=O)C(=C)COC(=O)CC(C)=O)cc(c1OCCCC)Cc1cc(CCCOC(=O)C(=C)COC(=O)CC(C)=O)cc(c1OCCCC)C2]

### Example 49

The same procedure as in Example 46 was repeated except that the compound obtained in Synthesis Example 80 (2.00 g, 1.44 mmol) was used instead of the compound obtained in Synthesis Example 77, to thereby obtain the target compound 010-7 (1.866 g, yield 75.1%).

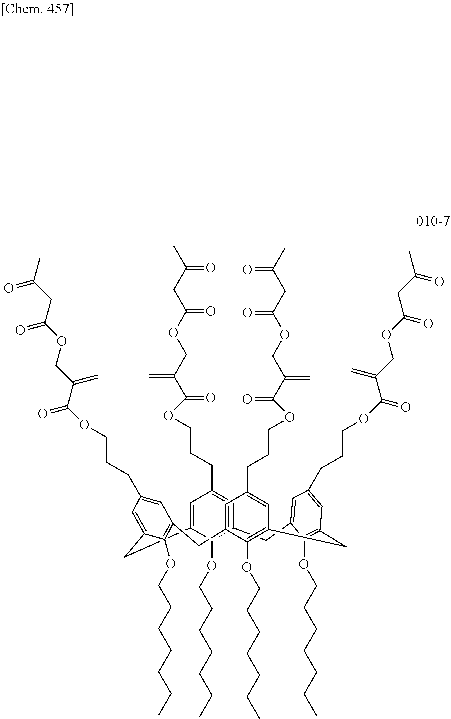 [SMILES: C=C(COC(=O)CC(C)=O)C(=O)OCCCc1cc2c(OCCCCCCC)c(c1)Cc1cc(CCCOC(=O)C(=C)COC(=O)CC(C)=O)cc(c1OCCCCCCC)Cc1cc(CCCOC(=O)C(=C)COC(=O)CC(C)=O)cc(c1OCCCCCCC)Cc1cc(CCCOC(=O)C(=C)COC(=O)CC(C)=O)cc(c1OCCCCCCC)C2]

### Example 50

The same procedure as in Example 46 was repeated except that the compound obtained in Synthesis Example 81 (2.00 g, 1.00 mmol) was used instead of the compound obtained in Synthesis Example 77, to thereby obtain the target compound 010-18 (1.570 g, yield 70.2%).

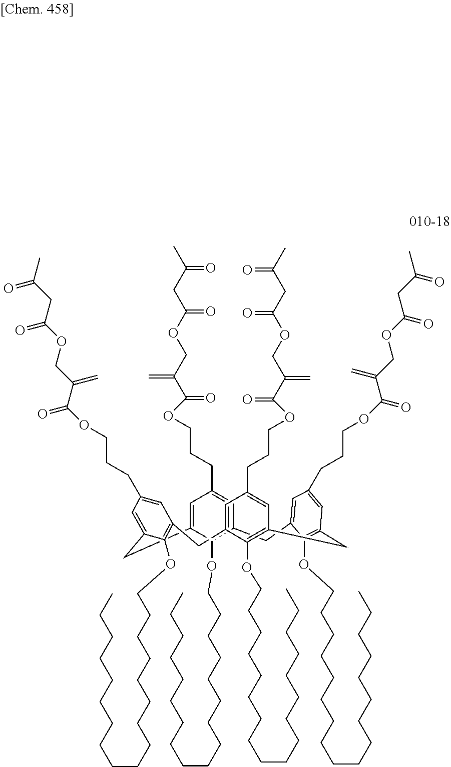

### COMPARATIVE EXAMPLE

A 100 mL four-necked flask equipped with a stirrer, a thermometer, and a reflux condenser tube was charged with 1.00 g (1.212 mmol) of the compound obtained in Synthesis Example 20, 10.00 g of tetrahydrofuran, 1.907 g (7.271 mmol) of triphenylphosphine, and 0.6260 g (7.271 mmol) of methacrylic acid, and the mixture was stirred. A light yellow transparent solution. Then 1.470 g (7.271 mmol) of diisopropyl azodicarboxylate was added dropwise to the solution placed in an ice bath over 30 minutes. A light yellow transparent solution. The light yellow transparent solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting orange viscus liquid was purified by column chromatography (eluent: n-hexane:acetone=90:10) to thereby obtain compound (1′) represented by the following formula. Compound (1′) was vacuum dried (at 60° C. for 6 hours or longer). 0.9058 g, and the yield was 68.1%.

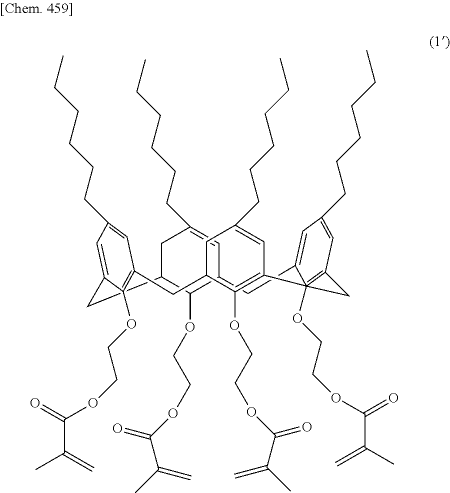

<Production of Curable Compositions>

0.25 g of one of the obtained calixarene compounds, 0.25 g of dipentaerythritol hexaacrylate (“A-DPH” manufactured by Shin Nakamura Chemical Co., Ltd.), 0.005 g of a polymerization initiator (“Irgacure 369” manufactured by BASF), and 9.5 g of propylene glycol monomethyl ether acetate were mixed to obtain a curable composition.

<Production of Layered Bodies>

The curable composition was applied to substrates 1 to 4 below by a spin coating method such that the thickness of the coating after curing was about 0.5 μm and then dried on a hot plate at 100° C. for 2 minutes. A high-pressure mercury lamp was used to irradiate the curable composition with UV rays at 500 mJ/cm² in a nitrogen atmosphere to cure the curable composition, and layered bodies were thereby obtained.

Substrate 1: polymethyl methacrylate resin plate

Substrate 2: aluminum plate

Substrate 3: polyethylene terephthalate film having a SiO₂ thin layer (thickness 100 nm) (the curable composition was applied to the SiO₂ thin film)

<Evaluation of Adhesion>

A layered body stored in a 23° C. and 50% RH environment for 24 hours was used, and the adhesion was evaluated according to JIS K6500-5-6 (adhesive strength: a cross-cut method). A cellophane tape used was “CT-24” manufactured by Nichiban Co., Ltd. The criteria for the evaluation are as follows.

A: 80 or more out of 100 squares were not detached and remained present.

B: 50 to 79 out of 100 squares were not detached and remained present.

C: The number of squares that were not detached and remained present was 49 or less out of 100 squares.

<Evaluation of Moist Heat Resistance>

One of the curable compositions was applied to a 5 inch SiO substrate to a film thickness of about 50 μm using an applicator and dried on a hot plate at 100° C. for 2 minutes. A mask having an LS pattern with L/S=50 μm/50 μm was brought into tight contact with the coating obtained, and a high-pressure mercury lamp was used to irradiate the composition with UV rays at 1000 mJ/cm² in a nitrogen atmosphere to cure the composition. The substrate exposed to the light was developed using ethyl acetate to obtain an evaluation substrate. The obtained substrate was stored in a thermo-hygrostat at 85° C. and 85% RH for 100 hours, and a laser microscope (“VK-X200” manufactured by KEYENCE CORPORATION) was used to check the state of the pattern after a lapse of 100 hours. The criteria for the evaluation are as follows.

A: The entire pattern was well modified and maintained.

B: Cracking or chipping was observed in part of the pattern.

C: Cracking or chipping was observed in the pattern, and delamination of the pattern was also observed.

TABLE 16

| Calixarene compound | | 1-6 | 2-6 | 3-6 | 4-6 | 1-4 | 2-4 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 3-4 | 4-4 | 1-7 | 2-7 | 3-7 | 4-7 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 5-6 | 6-6 | 7-6 | 8-6 | 9-6 | 10-6 |
| Adhesion | Substrate 1 | A | B | B | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 17

| Calixarene compound | | 11-6 | 12-6 | 9-4 | 10-4 | 11-4 | 12-4 |
|---|---|---|---|---|---|---|---|
| Adhesion | Substrate 1 | A | B | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 9-7 | 10-7 | 11-7 | 12-7 | 13-6 | 14-6 |
|---|---|---|---|---|---|---|---|
| Adhesion | Substrate 1 | A | A | A | B | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | B |

| Calixarene compound | | 15-6 | 16-6 | 17-6 | 18-6 | 19-6 | 20-6 |
|---|---|---|---|---|---|---|---|
| Adhesion | Substrate 1 | B | B | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 18

| Calixarene compound | | 17-4 | 18-4 | 19-4 | 20-4 | 17-7 | 18-7 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 19-7 | 20-7 | 17-18 | 18-18 | 19-18 | 20-18 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 17-1 | 18-1 | 19-1 | 20-1 | 21-6 | 22-6 |
| Adhesion | Substrate 1 | A | A | B | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 19

| Calixarene compound | | 23-6 | 24-6 | 25-6 | 26-6 | 27-6 | 28-6 |
|---|---|---|---|---|---|---|---|
| Adhesion | Substrate 1 | B | B | A | A | A | B |
| | Substrate 2 | A | A | A | A | A | A |
| | Substrate 3 | A | A | A | A | A | A |
| Moist heat resistance | | A | B | A | A | A | B |
| Calixarene compound | | 29-6 | 30-6 | 31-6 | 32-6 | 33-6 | 34-6 |
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | B | A | A |
| Calixarene compound | | 35-6 | 36-6 | 37-6 | 38-6 | 39-6 | 40-6 |
| Adhesion | Substrate 1 | A | A | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | B | A | A | A | A |

TABLE 20

| Calixarene compound | | 41-6 | 42-6 | 43-6 | 44-6 | 45-6 | 46-6 |
|---|---|---|---|---|---|---|---|
| Adhesion | Substrate 1 | A | A | A | B | A | B |
| | Substrate 2 | A | A | A | A | A | A |
| | Substrate 3 | A | A | A | A | A | A |
| Moist heat resistance | | A | A | A | A | A | A |
| Calixarene compound | | 47-6 | 48-6 | 49-6 | 50-6 | 51-6 | 52-6 |
| Adhesion | Substrate 1 | B | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 53-6 | 54-6 | 55-6 | 56-6 | 57-6 | 57-4 |
| Adhesion | Substrate 1 | A | B | B | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 21

| Calixarene compound | | 57-7 | 57-18 | 57-1 | 58-6 | 59-6 | 60-6 |
|---|---|---|---|---|---|---|---|
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 01-6 | 02&03-6 | 04-6 | 01-1 | 02&03-1 | 04-1 |
|---|---|---|---|---|---|---|---|
| Adhesion | Substrate 1 | A | A | B | A | A | B |
| | Substrate 2 | A | A | A | A | A | A |
| | Substrate 3 | A | A | A | A | A | A |
| Moist heat resistance | | A | A | A | A | A | A |
| Calixarene compound | | 01-4 | 02&03-4 | 04-4 | 01-7 | 02&03-7 | 04-7 |
| Adhesion | Substrate 1 | A | A | B | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 22

| Calixarene compound | | 01-18 | 02&03-18 | 04-18 | 05-6 | 05-1 | 05-4 |
|---|---|---|---|---|---|---|---|
| Adhesion | Substrate 1 | A | A | B | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 05-7 | 05-18 | 06-6 | 07&08-6 | 09-6 | 06-1 |
|---|---|---|---|---|---|---|---|
| Adhesion | Substrate 1 | A | A | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 07&08-1 | 09-1 | 06-4 | 07&08-4 | 09-4 | 06-7 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 23

| Calixarene compound | | 07&08-7 | 09-7 | 06-18 | 07&08-18 | 09-18 | 010-6 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 010-1 | 010-4 | 010-7 | 010-18 | 1′ |
|---|---|---|---|---|---|---|
| Adhesion | Substrate 1 | A | A | A | A | C |
| | Substrate 2 | A | A | A | A | C |
| | Substrate 3 | A | A | A | A | C |

| | | | | | |
|---|---|---|---|---|---|
| Moist heat resistance | A | A | A | A | B |

### Example Group <IV>

### Synthesis Example 1

A 20 L separable four-necked flask equipped with a stirrer, a thermometer, and a reflux condenser tube was quickly charged with 1000 g (1.54 mol) of t-butyl-calix[4]arene, 1159 g (12.32 mol) of phenol, and 9375 mL of dehydrated toluene, and the mixture was stirred at 300 rpm under nitrogen flow. The t-butyl-calix[4]arene used as a raw material was not dissolved but was suspended. Then, while the flask was cooled in an ice bath, 1643 g (12.32 mol) of anhydrous aluminum chloride (III) was added in several portions. The solution turned into a light orange transparent solution, and the anhydrous aluminum chloride (III) precipitated on the bottom. The mixture was allowed to react at room temperature for 5 hours. Then the contents were transferred to a 1 L beaker, and 20 kg of ice, 10 L of 1N hydrochloric acid, and 20 L of chloroform were added to stop the reaction. The mixture turned into a light yellow transparent solution. This reaction mixture was transferred to a separatory funnel, and the organic layer was separated. Then the aqueous layer was extracted with 5 L of chloroform three times, and the extract was combined with the organic layer. The organic layer was pre-dried over anhydrous magnesium sulfate and filtered. The solvent was removed by evaporation using an evaporator, and a mixture of white crystals and a colorless transparent solution was thereby obtained. Methanol was added slowly to the mixture under stirring to reprecipitate the crystals. The white crystals were filtered on a Kiriyama funnel and washed with methanol. The obtained white crystals were vacuum dried (at 50° C. for 6 hours or longer) to thereby obtain 597 g of the target intermediate A. The yield was 91%.

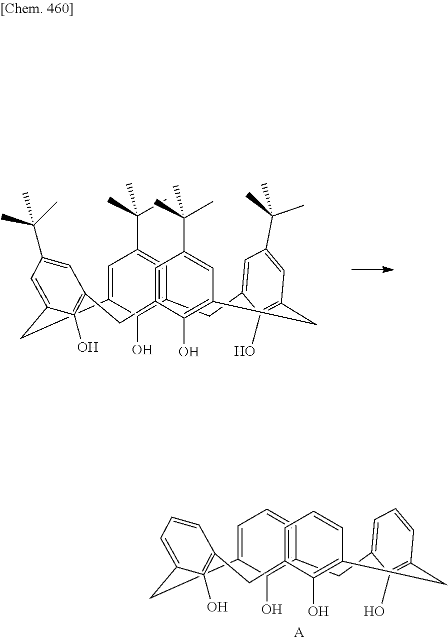

### Synthesis Example 2

A 2 L four-necked flask equipped with a stirrer, a thermometer, and a reflux condenser tube was charged with 205 g (1.52 mol) of n-hexanoyl chloride and 709 g of nitroethane, and the mixture was stirred. Then, while the flask was cooled in an ice bath, 243 g (1.82 mol) of anhydrous aluminum chloride (III) was added in several portions. The solution turned into a light orange transparent solution. The resulting solution was stirred at room temperature for 30 minutes, and 100 g (0.236 mol) of intermediate A was added in several portions. The mixture foamed and turned into an orange transparent solution. The solution was allowed to react at room temperature for 5 hours. Then the contents were slowly transferred to a 2 L beaker containing 450 mL of chloroform and 956 g of ice water to stop the reaction. Then 1N hydrochloric acid was added until the pH was 1. The reaction mixture was transferred to a separatory funnel, and the organic layer was separated. Next, the aqueous layer was extracted with 400 mL of chloroform three times, and the extract was combined with the organic layer. The organic layer was pre-dried over anhydrous magnesium sulfate. The solvent was removed by evaporation using an evaporator, and a yellow transparent solution was thereby obtained. While the solution was cooled in an ice bath, methanol was added to reprecipitate crystals. The white crystals were filtered on a Kiriyama funnel and recrystallized with chloroform and methanol. The obtained white crystals were vacuum dried (at 60° C. for 6 hours or longer) to thereby obtain 122 g of compound B-6 represented by the following structural formula. The yield was 63%.

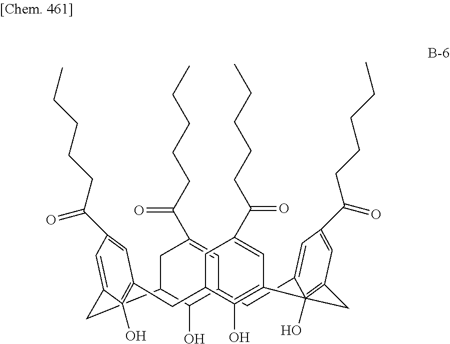

### Synthesis Example 3

The same procedure as in Synthesis Example 2 was repeated except that butyl chloride was used instead of n-hexanoyl chloride, to thereby obtain 106 g of compound B-4 represented by the following structural formula. The yield was 64%.

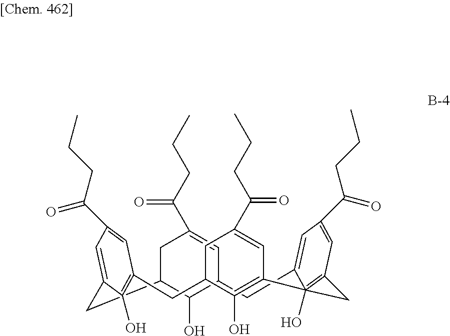

### Synthesis Example 4

The same procedure as in Synthesis Example 2 was repeated except that n-heptanoyl chloride was used instead of n-hexanoyl chloride, to thereby obtain 134 g of compound B-7 represented by the following structural formula. The yield was 65%.

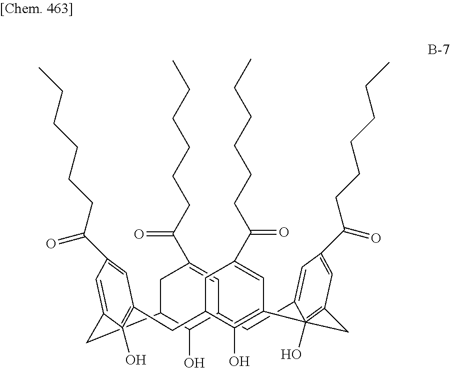

### Synthesis Example 5

The same procedure as in Synthesis Example 2 was repeated except that stearoyl chloride was used instead of n-hexanoyl chloride, to thereby obtain 228 g of compound B-18 represented by the following structural formula. The yield was 65%.

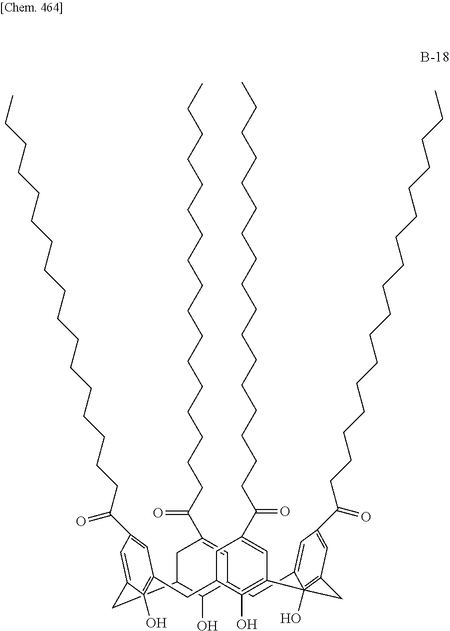

### Synthesis Example 6

A 100 mL four-necked flask equipped with a stirrer, a thermometer, and a reflux condenser tube was charged with 5.00 g (6.119 mmol) of B-6, 17.0 g of acetonitrile, 11.28 g (48.95 mmol) of potassium carbonate, 0.813 g (4.896 mmol) of potassium iodide, and 7.489 g (48.95 mmol) of methyl 2-bromoacetate, and the mixture was allowed to react at 70° C. for 24 hours. The resulting mixture was cooled to room temperature, and ion exchanged water and 0.3N hydrochloric acid were added until the pH was 6. Then 50 g of chloroform was added. The reaction mixture was transferred to a separatory funnel, and the organic layer was separated. Next, the aqueous layer was extracted with 50 g of chloroform three times, and the extract was combined with the organic layer. The organic layer was pre-dried over anhydrous magnesium sulfate, and the solvent was removed by evaporation using an evaporator to thereby obtain a red waxy solid. The obtained red waxy solid was vacuum dried (at 60° C. for 6 hours or longer) to thereby obtain 5.04 g of compound C-6 represented by the following structural formula. The yield was 74.5%.

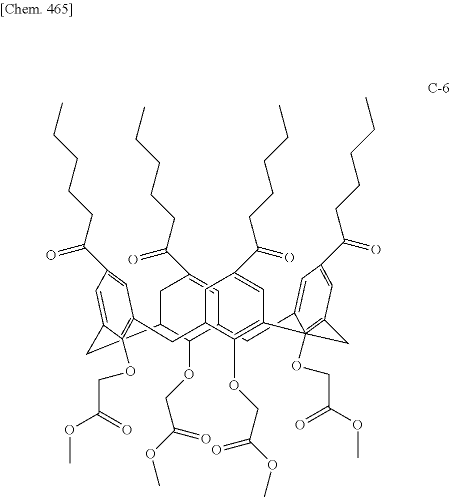

### Synthesis Example 7

The same procedure as in Synthesis Example 6 was repeated except that B-4 was used instead of B-6, to thereby obtain 4.88 g of compound C-4 represented by the following structural formula with a yield of 69.3%.

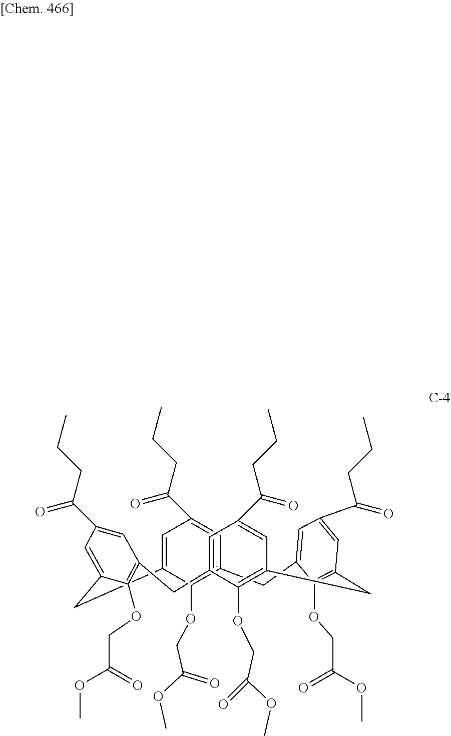 [SMILES: CCCC(=O)c1cc2c(OCC(=O)OC)c(c1)Cc1cc(C(=O)CCC)cc(c1OCC(=O)OC)Cc1cc(C(=O)CCC)cc(c1OCC(=O)OC)Cc1cc(C(=O)CCC)cc(c1OCC(=O)OC)C2]

### Synthesis Example 8

The same procedure as in Synthesis Example 6 was repeated except that B-7 was used instead of B-6, to thereby obtain 5.12 g of compound C-7 represented by the following structural formula with a yield of 77.0%.

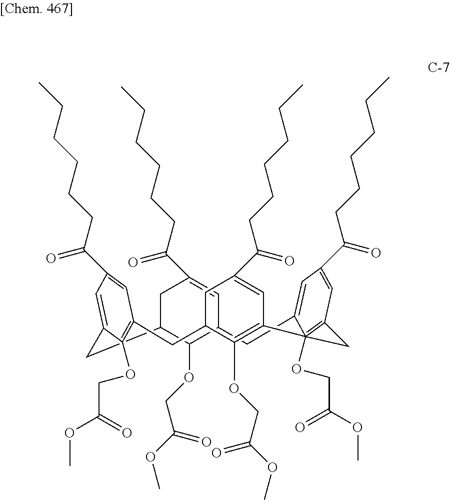

### Synthesis Example 9

The same procedure as in Synthesis Example 6 was repeated except that B-18 was used instead of B-6, to thereby obtain 5.34 g of compound C-18 represented by the following structural formula with a yield of 89.5%.

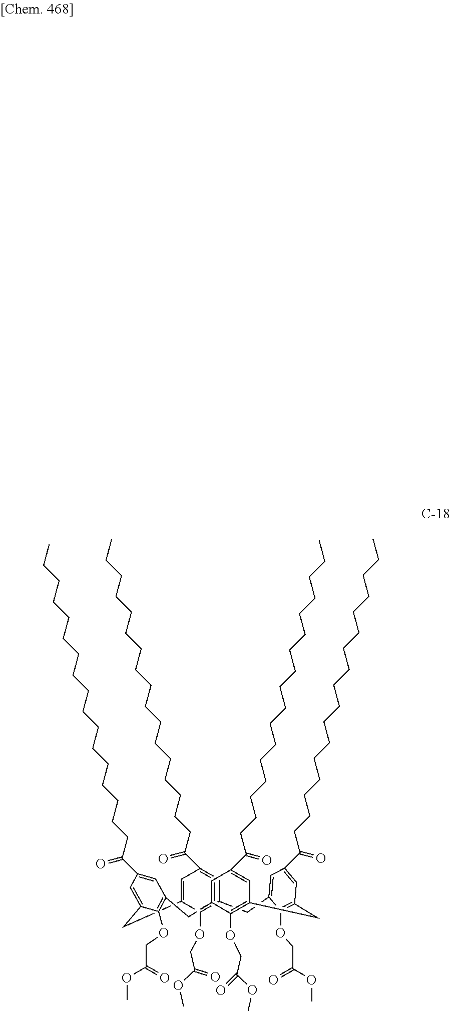 [SMILES: CCCCCCCCCCCCCCCCCC(=O)c1cc2c(OCC(=O)OC)c(c1)Cc1cc(C(=O)CCCCCCCCCCCCCCCCC)cc(c1OCC(=O)OC)Cc1cc(C(=O)CCCCCCCCCCCCCCCCC)cc(c1OCC(=O)OC)Cc1cc(C(=O)CCCCCCCCCCCCCCCCC)cc(c1OCC(=O)OC)C2]

### Synthesis Example 10

A 500 mL four-necked flask equipped with a stirrer, a thermometer, and a reflux condenser tube was charged with 16.44 g of tetrahydrofuran in an ice bath, and 1.038 g (27.35 mmol) of lithium aluminum hydride was slowly added. 5.04 g (4.559 mmol) of C-6 diluted with 49.31 g of tetrahydrofuran was added dropwise from a dropping funnel such that the temperature did not exceed 10° C. The resulting gray suspension reaction solution was allowed to react at room temperature for 6 hours. Then 30 g of chloroform was added to the resulting mixture placed in an ice bath, and 30 g of 5N hydrochloric acid was added dropwise to stop the reaction. Then diatomaceous earth was used to filter the reaction solution, and the filtrate was transferred to a separatory funnel to separate the organic layer. Next, the aqueous layer was extracted with 30 g of chloroform three times, and the extract was combined with the organic layer. The organic layer was pre-dried over anhydrous magnesium sulfate, and the solvent was removed by evaporation using an evaporator to thereby obtain a light yellow liquid. By-products were removed by column chromatography using an eluent: n-hexane:ethyl acetate=1:1, and then the target compound was eluted with an eluent: chloroform:isopropyl alcohol=5:1. The eluent was removed by evaporation under reduced pressure to thereby obtain 2.857 g of white solid compound D-6 represented by the following structural formula. The yield was 63.1%.

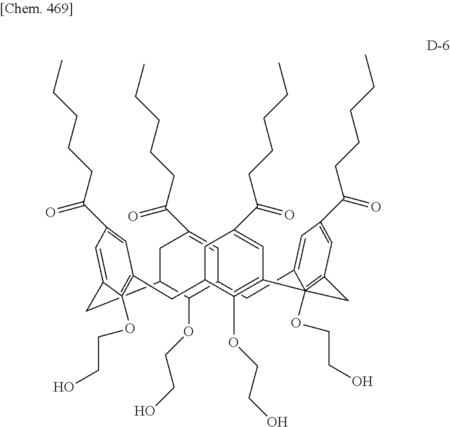

### Synthesis Example 11

The same procedure as in Synthesis Example 10 was repeated except that C-4 was used instead of C-6, to thereby obtain 3.06 g of compound D-4 represented by the following structural formula with a yield of 69.0%.

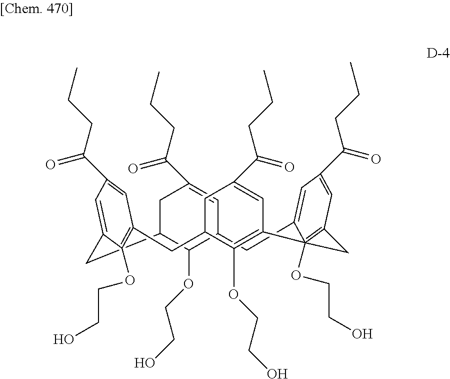

### Synthesis Example 12

The same procedure as in Synthesis Example 10 was repeated except that C-7 was used instead of C-6, to thereby obtain 3.11 g of compound D-7 represented by the following structural formula with a yield of 68.2%.

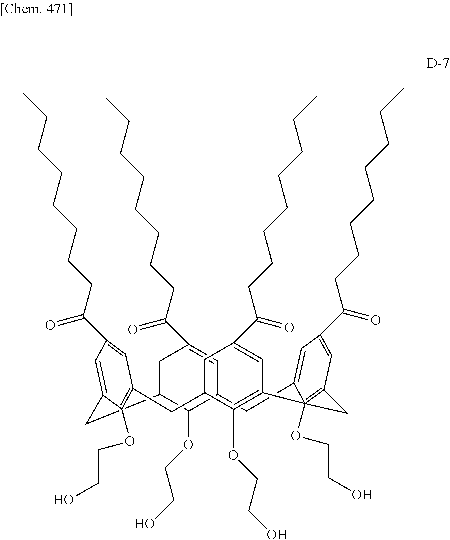

### Example 1

A 50 mL four-necked flask equipped with a stirrer, a thermometer, and a reflux condenser tube was charged with 1.00 g (1.01 mmol) of D-6, 2.90 g of tetrahydrofuran, and 0.74 g (6.04 mmol) methyl oxal chloride, and the mixture was stirred under cooling with ice. 0.61 g (6.04 mmol) of triethylamine dissolved in 1.20 g of tetrahydrofuran was added to the mixture, and the resulting mixture was stirred at room temperature for 3 hours. Water was added to the reaction solution to stop the reaction, and the resulting solution was extracted with ethyl acetate, washed with water and saturated brine, and dried over magnesium sulfate. The solvent was removed by evaporation using an evaporator, and the resulting red viscous liquid was purified by column chromatography (eluent: n-hexane:ethyl acetate=85:15) to thereby obtain 0.401 g of the target compound 1-6 with a yield of 30.2%, 0.277 g of the target compound 2-6 with a yield of 21.1%, 0.261 g of the target compound 3-6 with a yield of 19.9%, and 0.111 g of the target compound 4-6 with a yield of 8.59%.

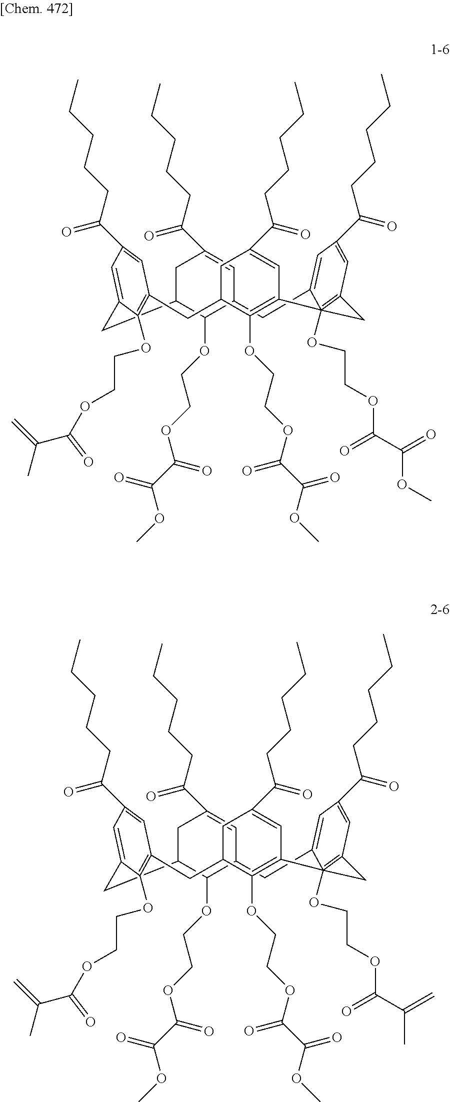

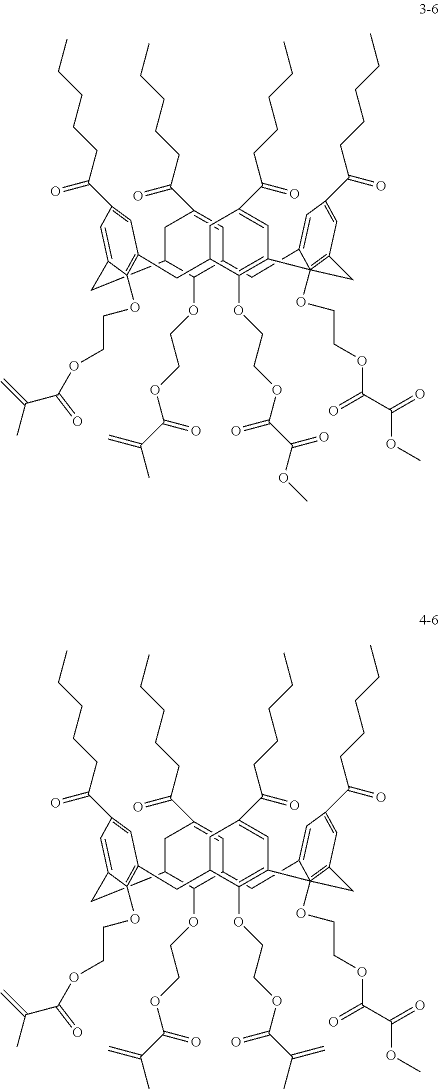

### Example 2

The same procedure as in Example 1 was repeated except that D-4 was used instead of D-6, to thereby obtain 0.387 g of the target compound 1-4 with a yield of 28.2%, 0.223 g of the target compound 2-4 with a yield of 16.5%, 0.243 g of the target compound 3-4 with a yield of 18.0%, and 0.113 g of the target compound 4-4 with a yield of 8.50%.

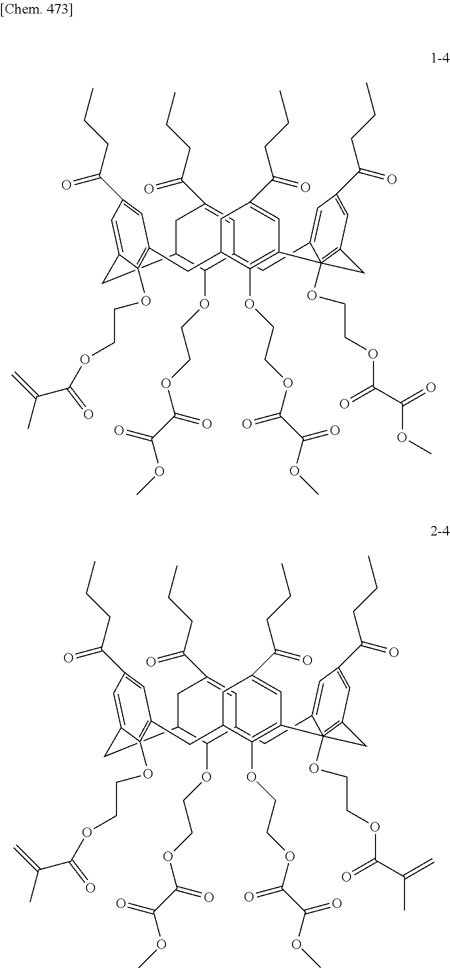

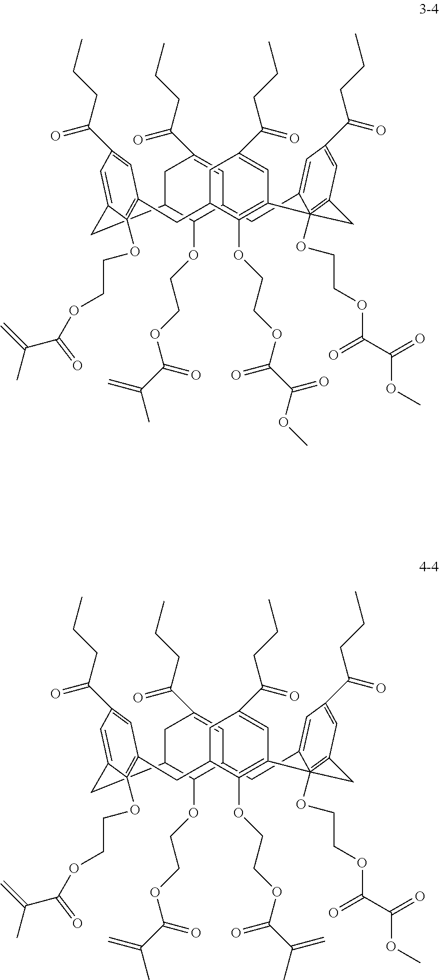

### Example 3

The same procedure as in Example 1 was repeated except that D-7 was used instead of D-6, to thereby obtain 0.412 g of the target compound 1-7 with a yield of 31.4%, 0.254 g of the target compound 2-7 with a yield of 19.6%, 0.234 g of the target compound 3-7 with a yield of 18.1%, and 0.121 g of the target compound 4-7 with a yield of 9.48%.

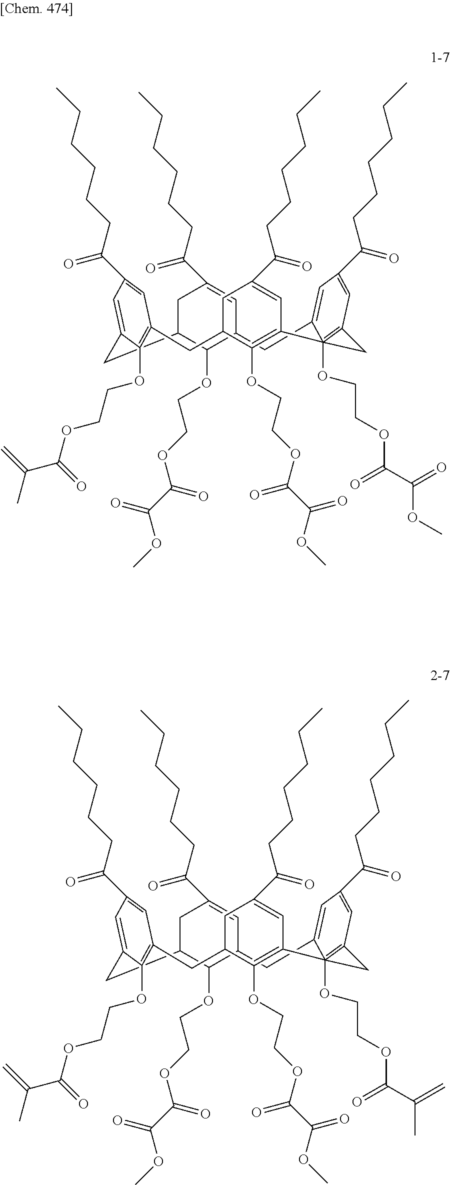

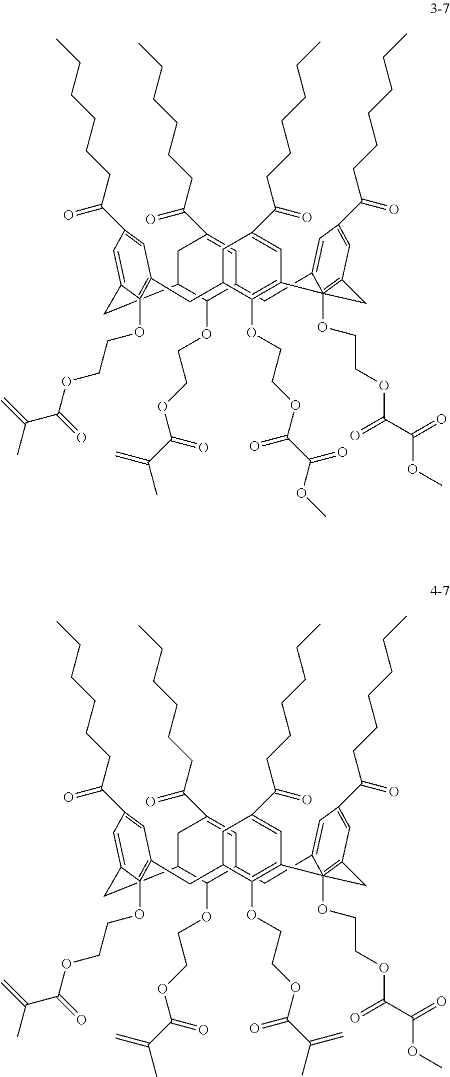

### Example 4

The same procedure as in Example 1 was repeated except that acrylic acid was used instead of methacrylic acid, to thereby obtain 0.401 g of the target compound 5-6 with a yield of 30.5%, 0.219 g of the target compound 6-6 with a yield of 17.1%, 0.207 g of the target compound 7-6 with a yield of 16.1%, and 0.105 g of the target compound 8-6 with a yield of 8.40%.

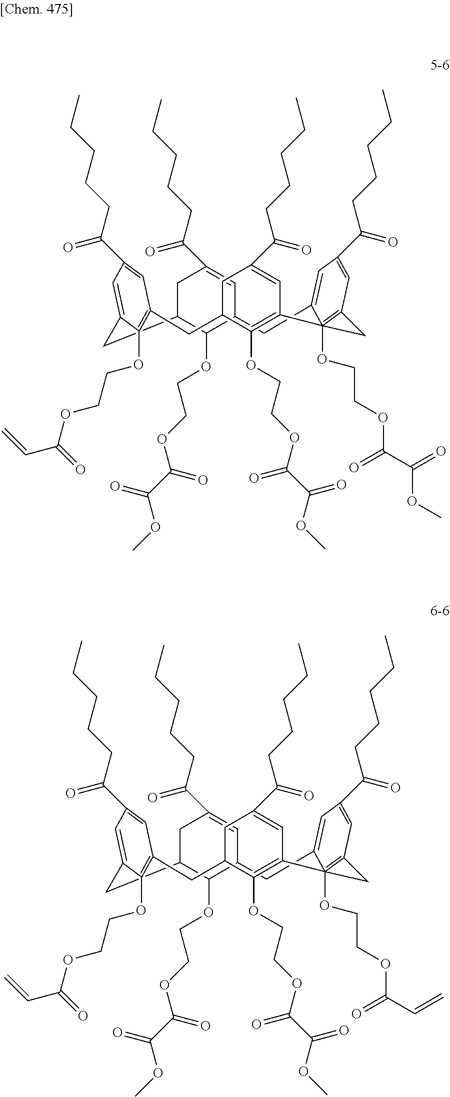

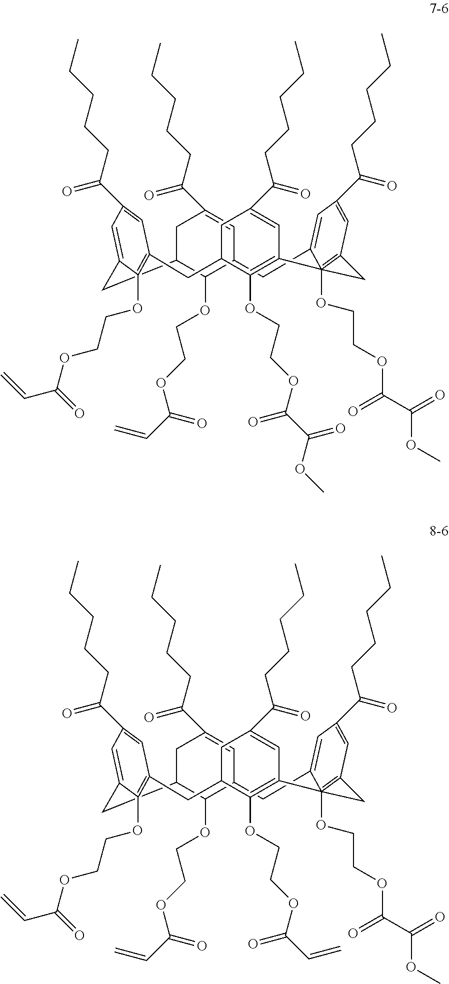

### Example 5

The same procedure as in Example 1 was repeated except that ethyl oxalyl chloride was used instead of methyl oxal chloride, to thereby obtain 0.421 g of the target compound 9-6 with a yield of 30.7%, 0.223 g of the target compound 10-6 with a yield of 16.7%, 0.208 g of the target compound 11-6 with a yield of 15.5%, and 0.113 g of the target compound 12-6 with a yield of 8.65%.

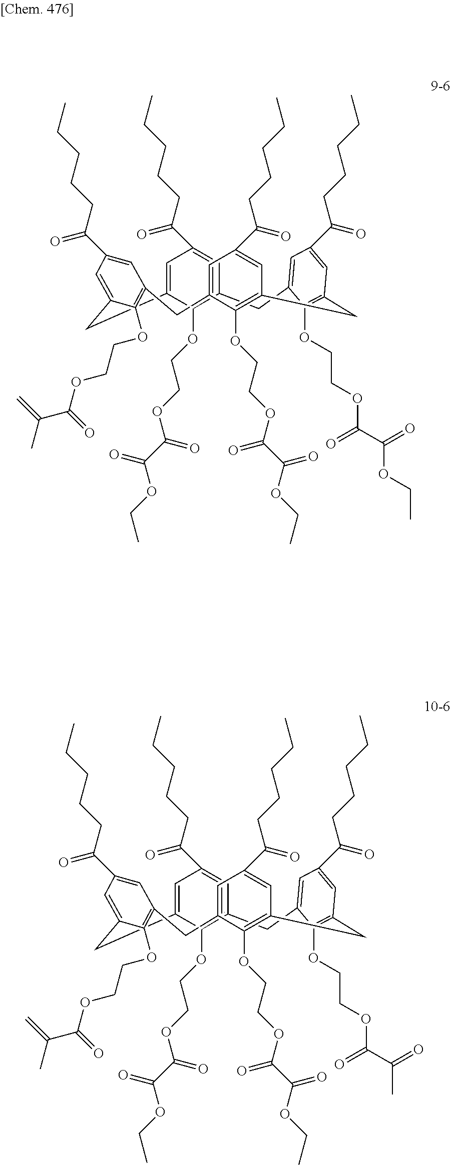

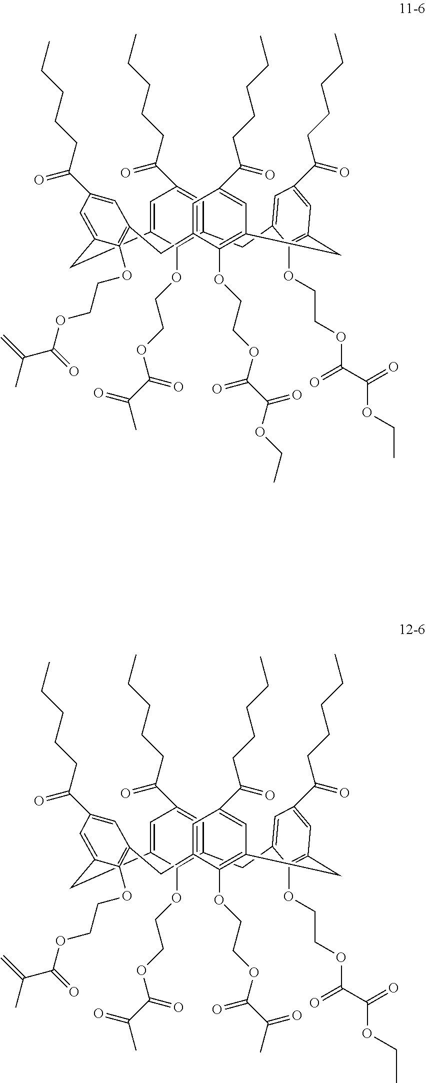

### Example 6

The same procedure as in Example 5 was repeated except that acrylic acid was used instead of methacrylic acid, to thereby obtain 0.411 g of the target compound 13-6 with a yield of 30.3%, 0.214 g of the target compound 14-6 with a yield of 16.3%, 0.218 g of the target compound 15-6 with a yield of 16.6%, and 0.120 g of the target compound 16-6 with a yield of 9.50%.

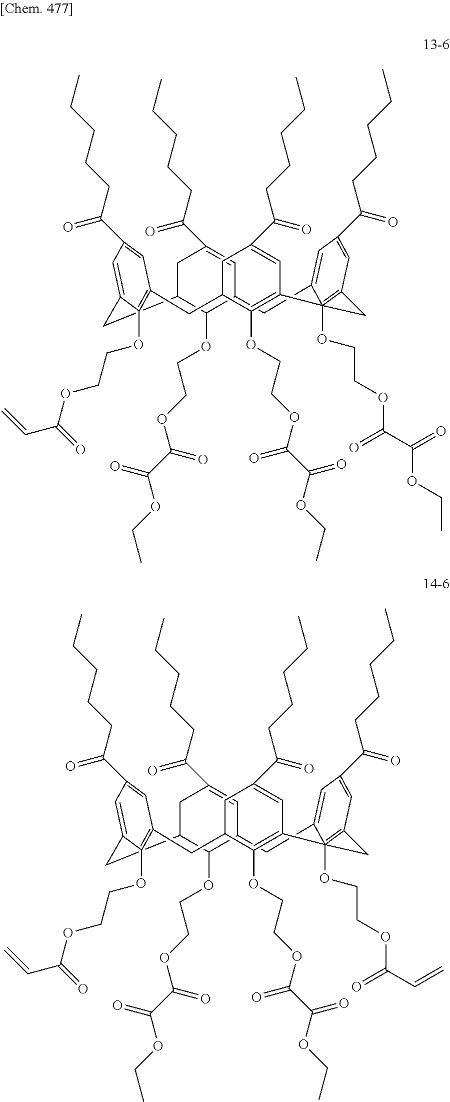

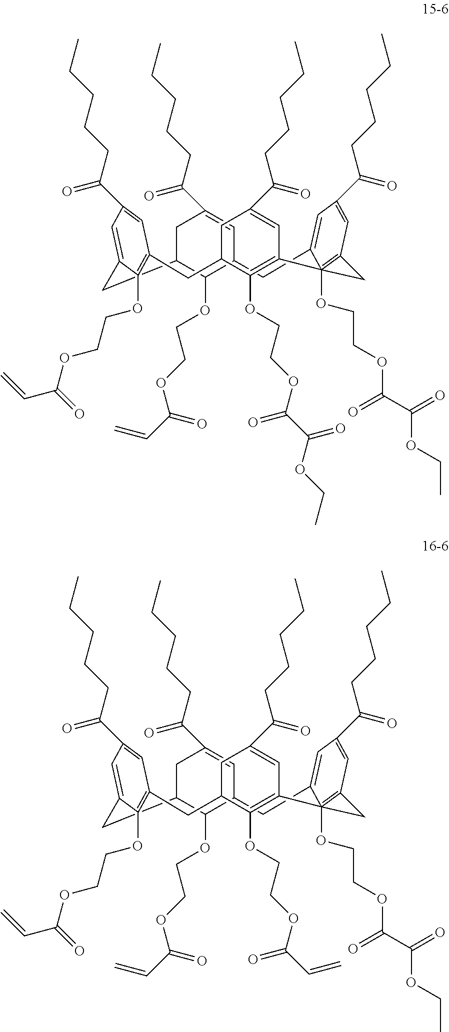

### Synthesis Example 13

The same procedure as in Synthesis Example 6 was repeated except that methyl bromopyopionate was used instead of methyl bromoacetate, to thereby obtain 4.307 g of compound E-6 represented by the following structural formula. The yield was 60.6%.

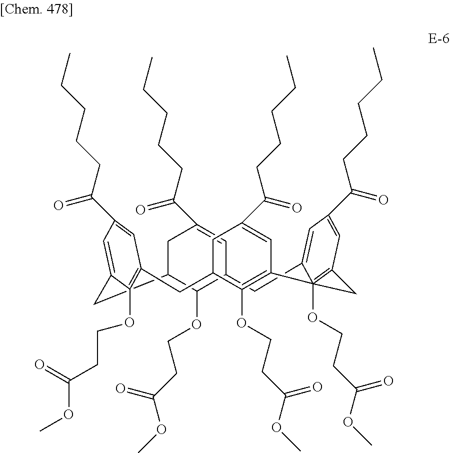

### Synthesis Example 14

The same procedure as in Synthesis Example 10 was repeated except that E-6 was used instead of C-6, to thereby obtain 2.989 g of compound F-6 represented by the following structural formula. The yield was 80.6%.

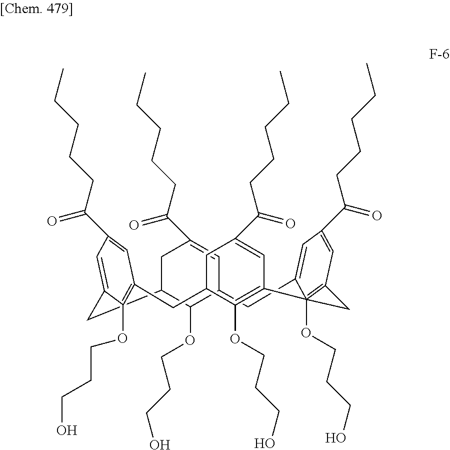

### Example 7

The same procedure as in Example 1 was repeated except that F-6 was used instead of D-6, to thereby obtain 0.387 g of the target compound 17-6 with a yield of 29.5%, 0.187 g of the target compound 18-6 with a yield of 14.4%, 0.176 g of the target compound 19-6 with a yield of 13.6%, and 0.093 g of the target compound 20-6 with a yield of 7.28%.

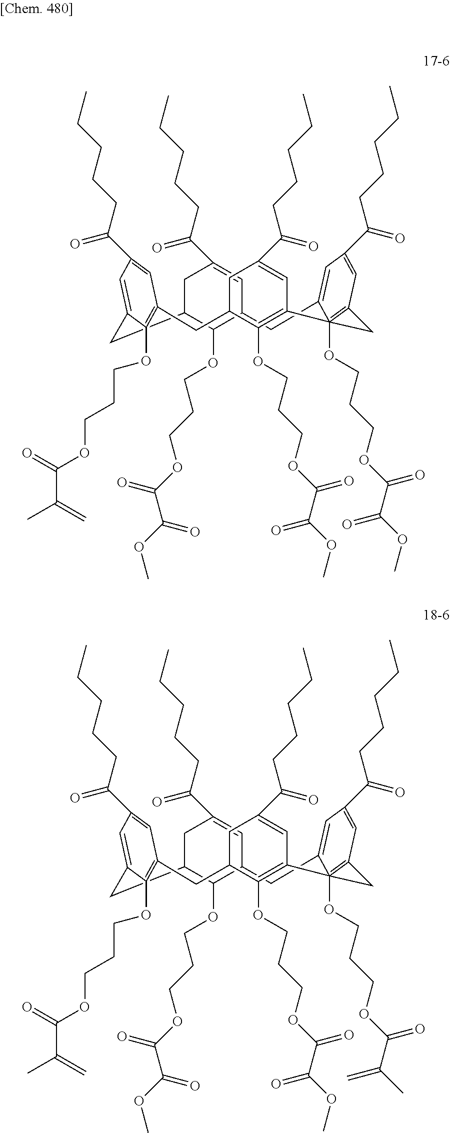

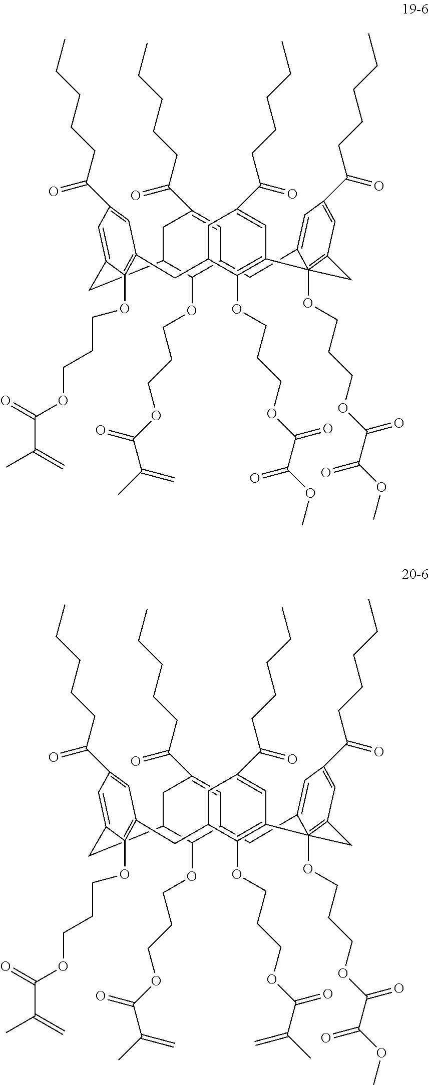

### Example 8

The same procedure as in Example 7 was repeated except that acrylic acid was used instead of methacrylic acid, to thereby obtain 0.376 g of the target compound 21-6 with a yield of 29.0%, 0.176 g of the target compound 22-6 with a yield of 13.9%, 0.17 g of the target compound 23-6 with a yield of 13.4%, and 0.089 g of the target compound 24-6 with a yield of 7.20%.

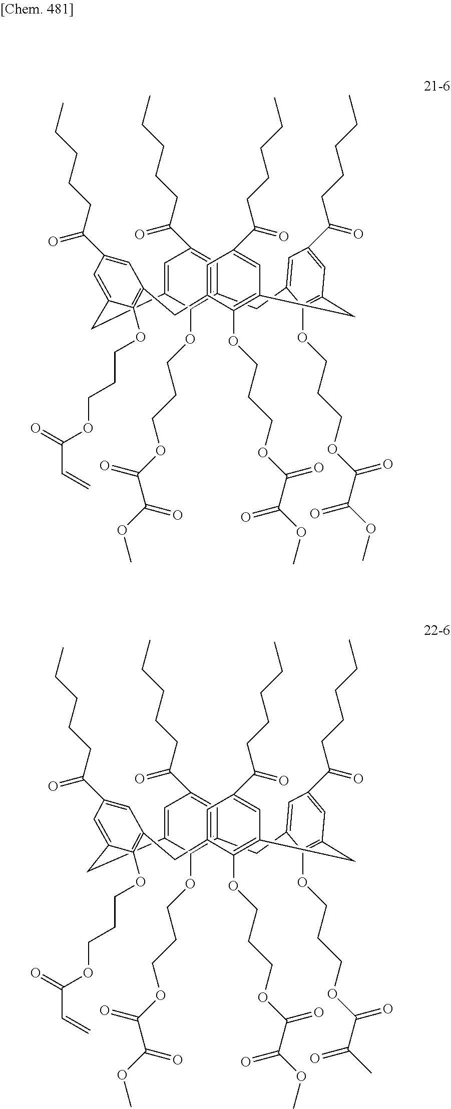 [SMILES: C=CC(=O)OCCCOc1c2cc(C(=O)CCCCC)cc1Cc1cc(C(=O)CCCCC)cc(c1OCCCOC(=O)C(=O)OC)Cc1cc(C(=O)CCCCC)cc(c1OCCCOC(=O)C(=O)OC)Cc1cc(C(=O)CCCCC)cc(c1OCCCOC(=O)C(=O)OC)C2 ; C=CC(=O)OCCCOc1c2cc(C(=O)CCCCC)cc1Cc1cc(C(=O)CCCCC)cc(c1OCCCOC(=O)C(=O)OC)Cc1cc(C(=O)CCCCC)cc(c1OCCCOC(=O)C(C)=O)Cc1cc(C(=O)CCCCC)cc(c1OCCCOC(=O)C(=O)OC)C2]

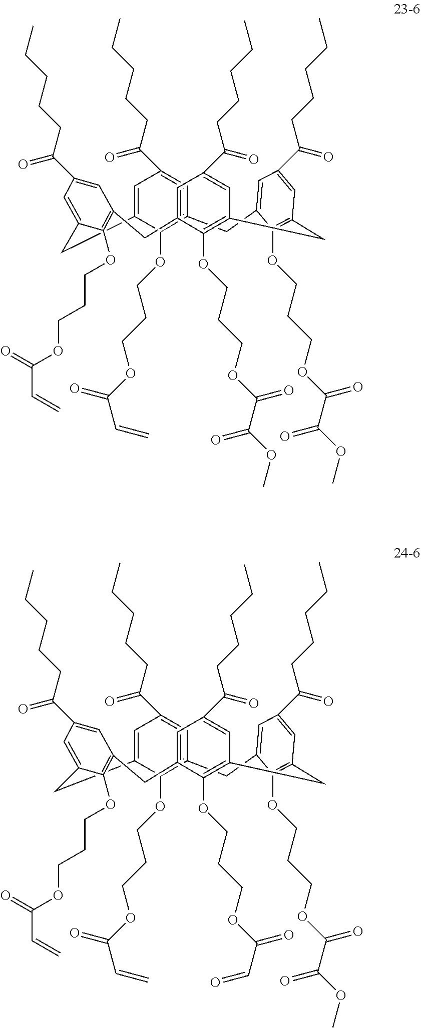 [SMILES: C=CC(=O)OCCCOc1c2cc(C(=O)CCCCC)cc1Cc1cc(C(=O)CCCCC)cc(c1OCCCOC(=O)C(=O)OC)Cc1cc(C(=O)CCCCC)cc(c1OCCCOC(=O)C(=O)OC)Cc1cc(C(=O)CCCCC)cc(c1OCCCOC(=O)C=C)C2 ; C=CC(=O)OCCCOc1c2cc(C(=O)CCCCC)cc1Cc1cc(C(=O)CCCCC)cc(c1OCCCOC(=O)C=O)Cc1cc(C(=O)CCCCC)cc(c1OCCCOC(=O)C(=O)OC)Cc1cc(C(=O)CCCCC)cc(c1OCCCOC(=O)C=C)C2]

### Example 9

The same procedure as in Example 7 was repeated except that ethyl oxalyl chloride was used instead of methyl oxal chloride, to thereby obtain 0.388 g of the target compound 25-6 with a yield of 28.7%, 0.201 g of the target compound 26-6 with a yield of 15.2%, 0.189 g of the target compound 27-6 with a yield of 14.3%, and 0.091 g of the target compound 28-6 with a yield of 7.05%.

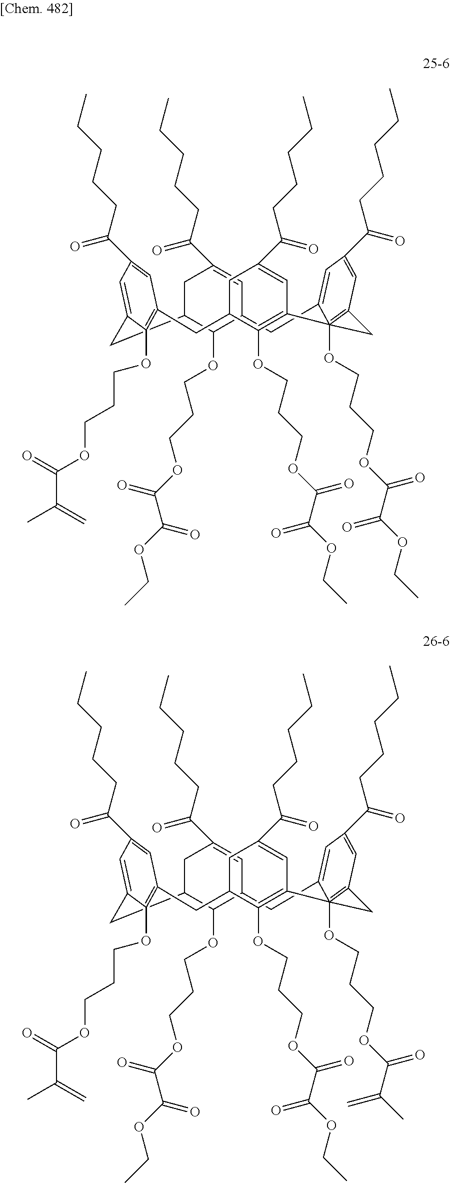

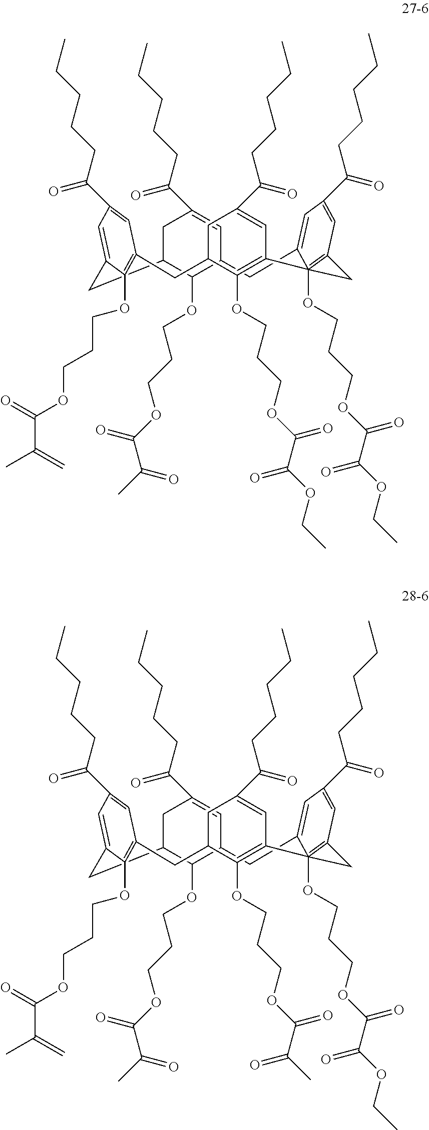

### Example 10

The same procedure as in Example 9 was repeated except that acrylic acid was used instead of methacrylic acid, to thereby obtain 0.386 g of the target compound 29-6 with a yield of 28.9%, 0.203 g of the target compound 30-6 with a yield of 15.7%, 0.197 g of the target compound 31-6 with a yield of 15.2%, and 0.100 g of the target compound 32-6 with a yield of 8.00%.

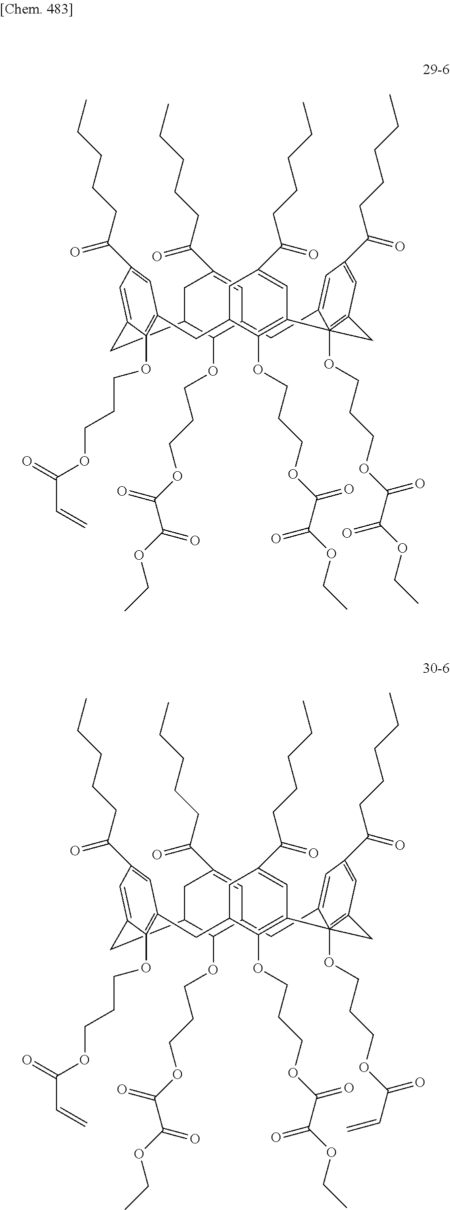

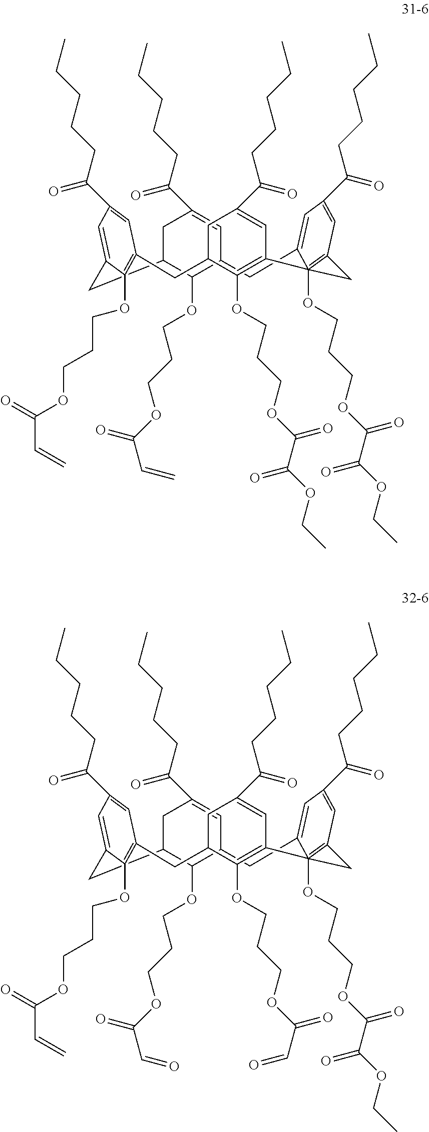

### Synthesis Example 15

A 500 mL four-necked flask equipped with a stirrer, a thermometer, and a reflux condenser tube was charged with 92.6 g (113.33 mmol) of B-6 and 944.52 g of diethylene glycol monomethyl ether, and the mixture was stirred. Then 46.4 mL (906.64 mmol) of hydrazine monohydrate and 50.9 g (906.64 mmol) of potassium hydroxide pellets were added, and the resulting mixture was stirred at 100° C. for 30 minutes. Then the mixture was further heat-refluxed for 8 hours. After completion of the reaction, the resulting mixture was cooled to 90° C., and 92.6 mL of ion exchanged water was added. Then the resulting mixture was cooled to room temperature. The solution mixture was transferred to a beaker. 6N hydrochloric acid was added until the pH was 1, and 300 g of chloroform was added. The reaction mixture was transferred to a separatory funnel, and the organic layer was separated. Next, the aqueous layer was extracted with 300 g of chloroform three times, and the extract was combined with the organic layer. The organic layer was pre-dried over anhydrous magnesium sulfate, and the solvent was removed by evaporation using an evaporator to thereby obtain an orange viscus liquid. Methanol was added to reprecipitate crystals, and the generated white crystals were filtered and vacuum dried (at 60° C. for 6 hours or longer) to thereby obtain 54.34 g of compound G-6 represented by the following structural formula. The yield was 63.0%.

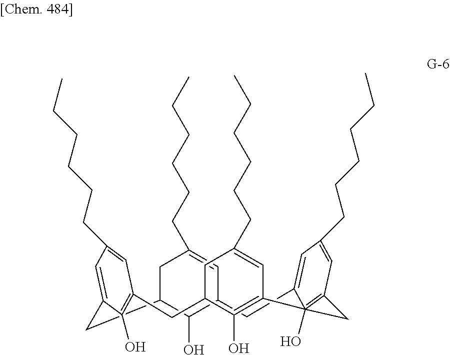

### Synthesis Example 16

The same procedure as in Synthesis Example 15 was repeated except that B-4 was used instead of B-6, to thereby obtain 72.45 g of compound G-4 represented by the following structural formula. The yield was 83.1%.

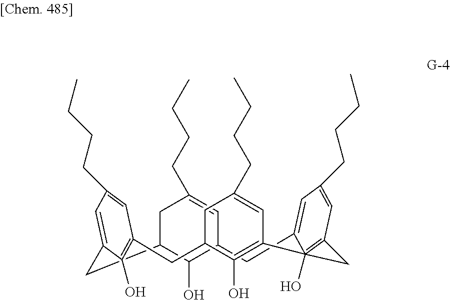

### Synthesis Example 17

The same procedure as in Synthesis Example 15 was repeated except that B-7 was used instead of B-6, to thereby obtain 78.4 g of compound G-7 represented by the following structural formula. The yield was 82.7%.

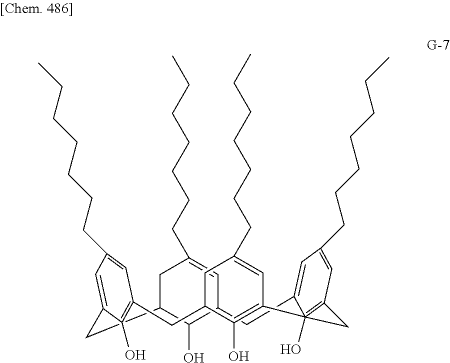

### Synthesis Example 18

The same procedure as in Synthesis Example 15 was repeated except that B-18 was used instead of B-6, to thereby obtain 37.9 g of compound G-18 represented by the following structural formula. The yield was 96.0%.

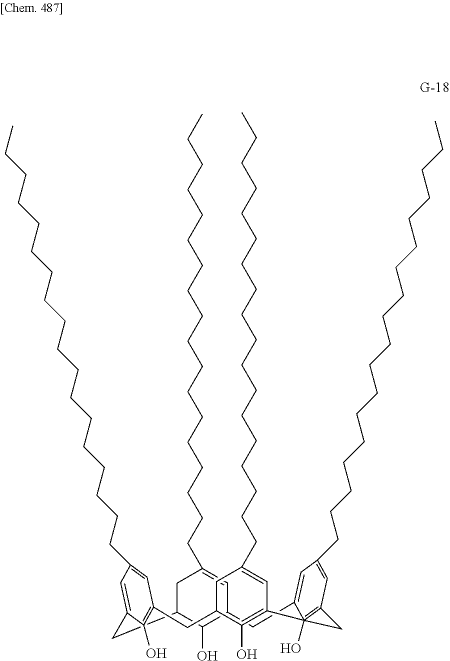

### Synthesis Example 19

Referring to known documents (Tetrahedron Letters, 43(43), 7691-7693; 2002 and Tetrahedron Letters, 48(5), 905-12; 1992), compound G-1 represented by the following structural formula was synthesized according to the following two-step scheme (yield amount 75 g, yield 66.6%).

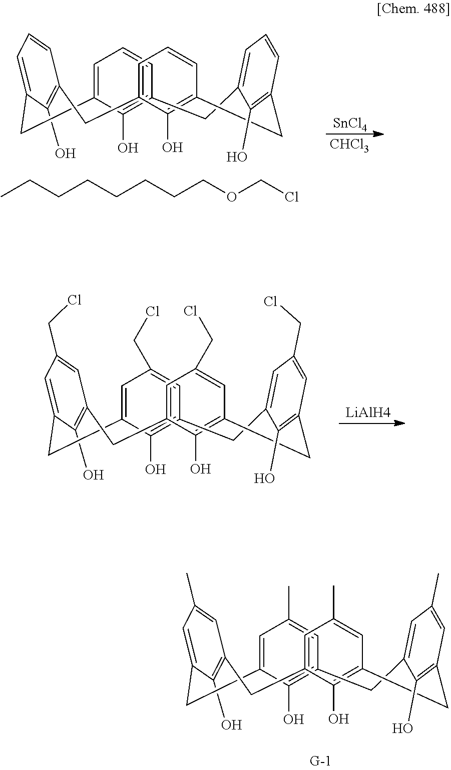

### Synthesis Example 20

A 1 L four-necked flask equipped with a stirrer, a thermometer, and a reflux condenser tube was charged with 20.00 g (26.276 mmol) of G-6, 400 g of acetonitrile, 15.29 g (105.11 mmol) of potassium carbonate, 10.511 g (10.511 mmol) of potassium iodide, and 32.158 g (210.21 mmol) of methyl 2-bromoacetate, and the mixture was allowed to react at 70° C. for 6 hours. The resulting mixture was cooled to room temperature, and ion exchanged water and 1N hydrochloric acid were added until the pH was 6. Then 500 g of chloroform was added. The reaction mixture was transferred to a separatory funnel, and the organic layer was separated. Next, the aqueous layer was extracted with 100 g of chloroform three times, and the extract was combined with the organic layer. The organic layer was pre-dried over anhydrous magnesium sulfate, and the solvent was removed by evaporation using an evaporator to thereby obtain a red waxy solid. The obtained red waxy solid was vacuum dried (at 60° C. for 6 hours or longer) to thereby obtain 21.67 g of compound H-6 represented by the following structural formula. The yield was 78.6%.

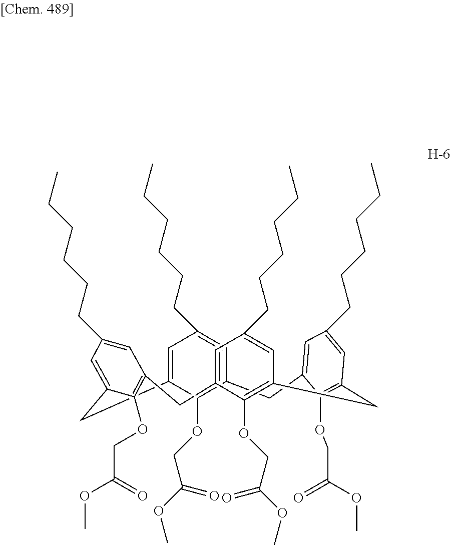 [SMILES: CCCCCCc1cc2c(OCC(=O)OC)c(c1)Cc1cc(CCCCCC)cc(c1OCC(=O)OC)Cc1cc(CCCCCC)cc(c1OCC(=O)OC)Cc1cc(CCCCCC)cc(c1OCC(=O)OC)C2]

### Synthesis Example 21

The same procedure as in Synthesis Example 20 was repeated except that G-4 was used instead of G-6, to thereby obtain 21.81 g of compound H-4 represented by the following structural formula. The yield was 75.5%.

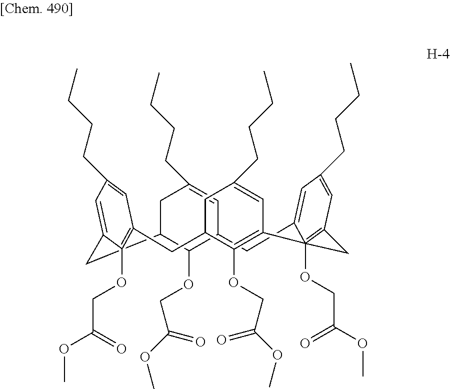

### Synthesis Example 22

The same procedure as in Synthesis Example 20 was repeated except that G-7 was used instead of G-6, to thereby obtain 20.98 g of compound H-7 represented by the following structural formula. The yield was 77.5%.

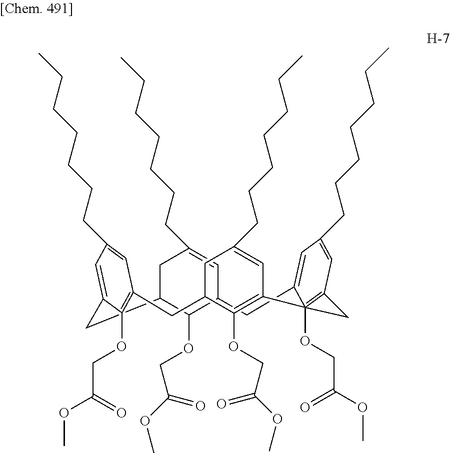

### Synthesis Example 23

The same procedure as in Synthesis Example 20 was repeated except that G-18 was used instead of G-6, to thereby obtain 19.32 g of compound H-18 represented by the following structural formula. The yield was 80.4%.

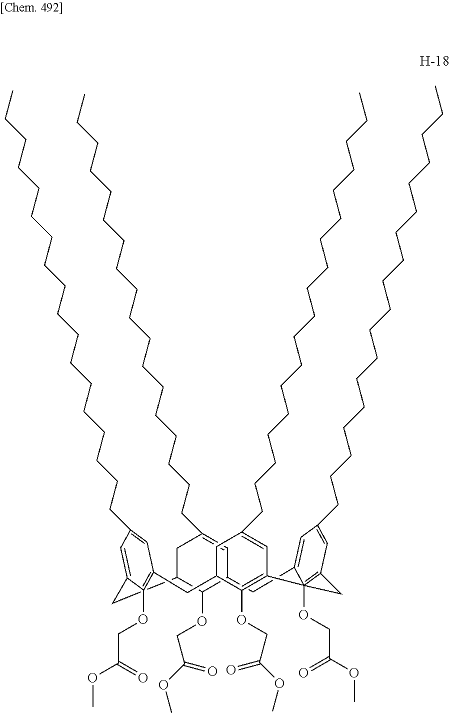

### Synthesis Example 24

The same procedure as in Synthesis Example 20 was repeated except that G-1 was used instead of G-6, to thereby obtain 18.32 g of compound H-1 represented by the following structural formula. The yield was 57.3%.

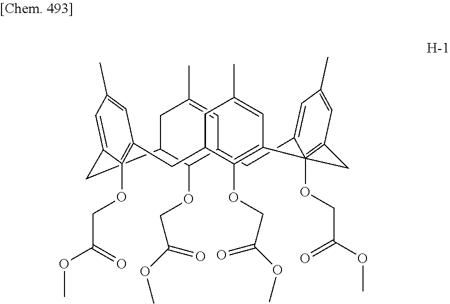

### Synthesis Example 25

The same procedure as in Synthesis Example 10 was repeated except that H-6 was used instead of C-6, to thereby obtain 6.12 g of compound I-6 represented by the following structural formula. The yield was 68.5%.

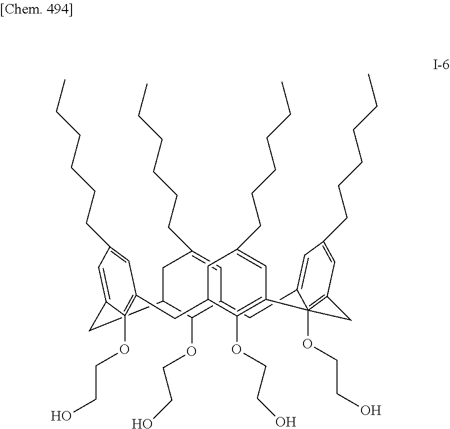

### Synthesis Example 26

The same procedure as in Synthesis Example 25 was repeated except that H-4 was used instead of H-6, to thereby obtain 4.21 g of compound I-4 represented by the following structural formula. The yield was 81.4%.

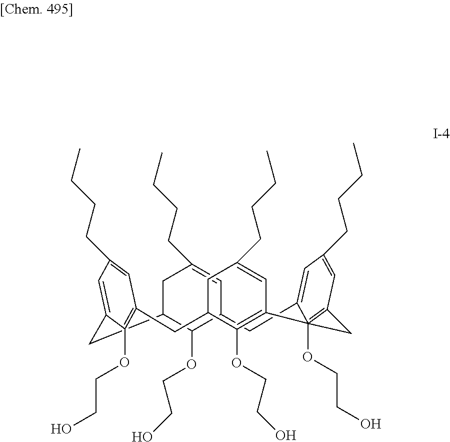

### Synthesis Example 27

The same procedure as in Synthesis Example 25 was repeated except that H-7 was used instead of H-6, to thereby obtain 3.89 g of compound I-7 represented by the following structural formula. The yield was 84.5%.

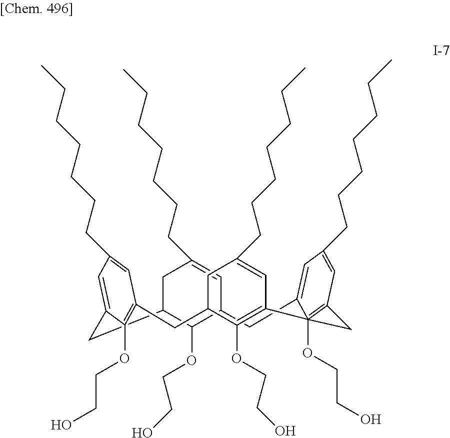

### Synthesis Example 28

The same procedure as in Synthesis Example 25 was repeated except that H-18 was used instead of H-6, to thereby obtain 4.31 g of compound I-18 represented by the following structural formula. The yield was 81.7%.

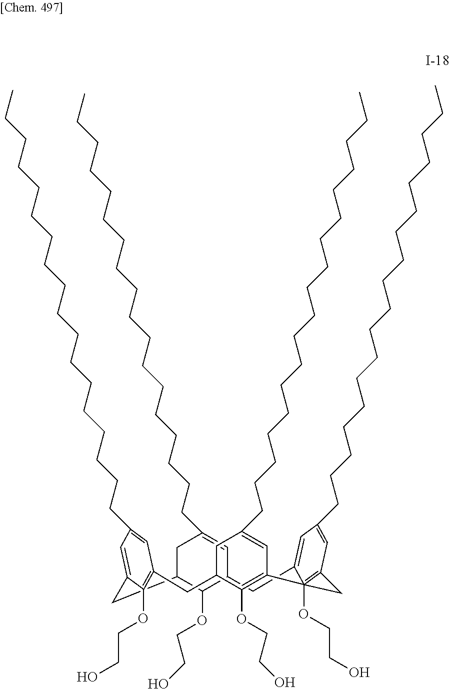

### Synthesis Example 29

The same procedure as in Synthesis Example 25 was repeated except that H-1 was used instead of H-6, to thereby obtain 3.43 g of compound I-1 represented by the following structural formula. The yield was 85.1%.

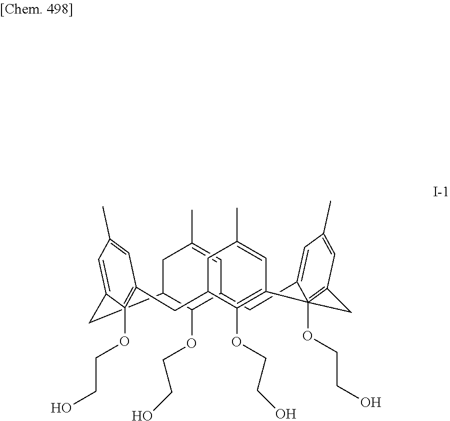

### Example 11

The same procedure as in Example 1 was repeated except that I-6 was used instead of D-6, to thereby obtain 0.421 g of the target compound 33-6 with a yield of 31.2%, 0.265 g of the target compound 34-6 with a yield of 19.9%, 0.251 g of the target compound 35-6 with a yield of 18.9%, and 0.131 g of the target compound 36-6 with a yield of 10.0%.

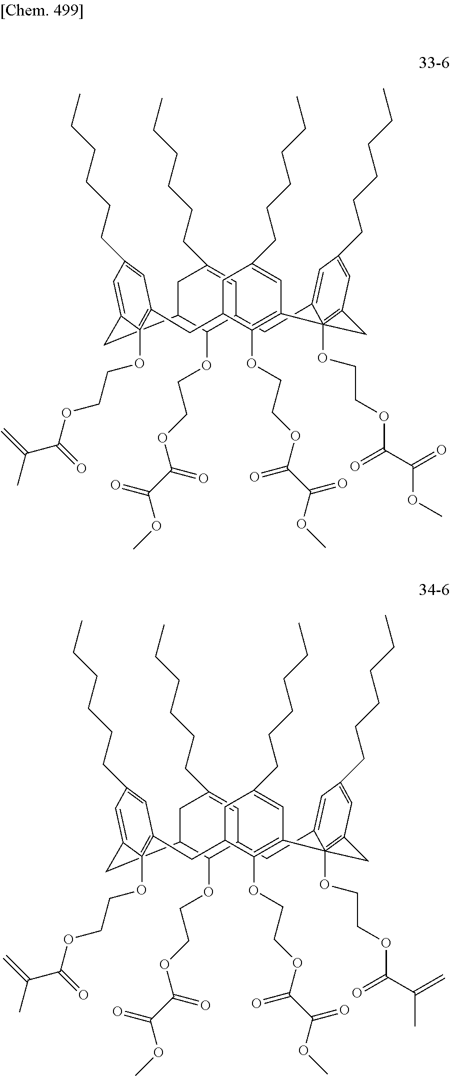

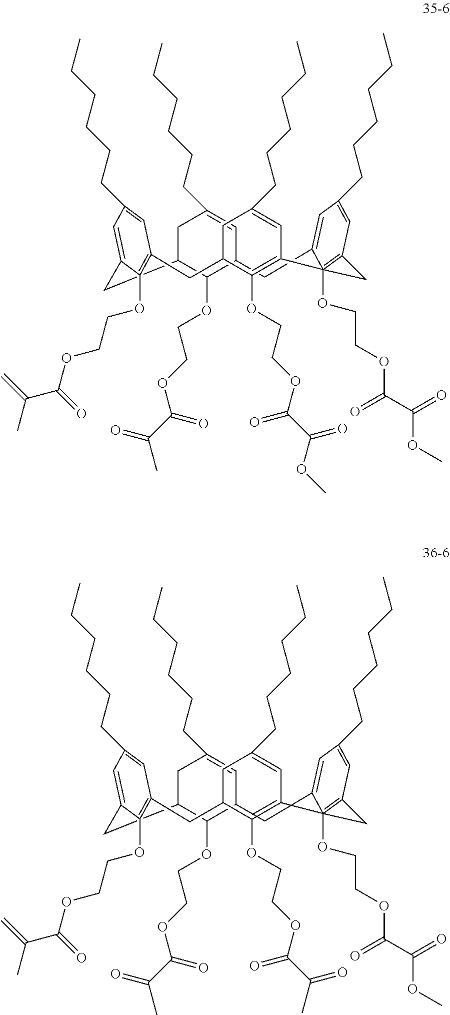

### Example 12

The same procedure as in Example 11 was repeated except that I-4 was used instead of I-6, to thereby obtain 0.42 g of the target compound 33-4 with a yield of 30.1%, 0.255 g of the target compound 34-4 with a yield of 18.6%, 0.239 g of the target compound 35-4 with a yield of 17.4%, and 0.126 g of the target compound 36-4 with a yield of 9.32%.

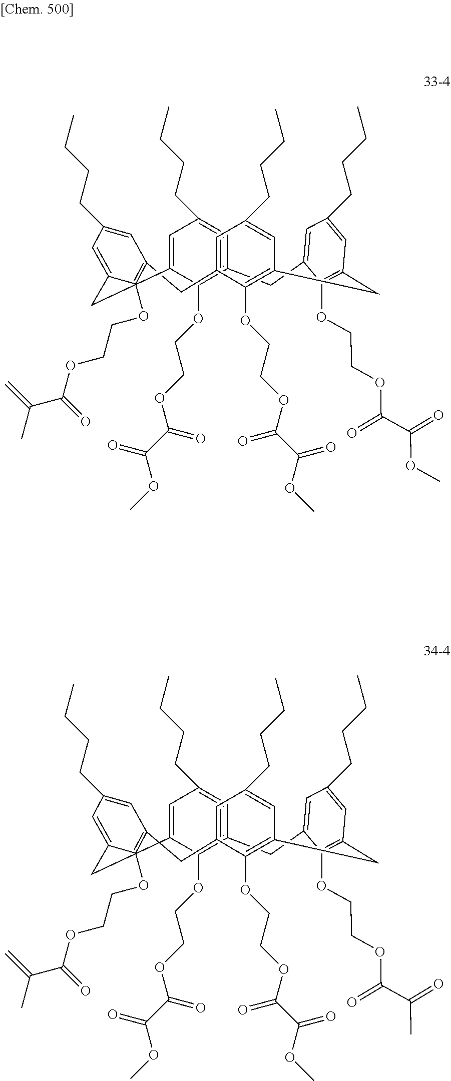

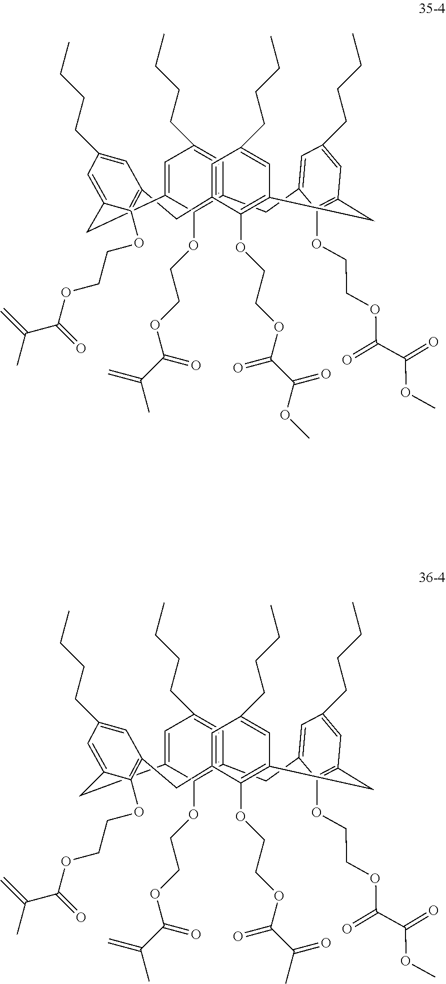

### Example 13

The same procedure as in Example 11 was repeated except that I-7 was used instead of I-6, to thereby obtain 0.411 g of the target compound 33-7 with a yield of 30.9%, 0.26 g of the target compound 34-7 with a yield of 19.8%, 0.255 g of the target compound 35-7 with a yield of 19.5%, and 0.123 g of the target compound 36-7 with a yield of 9.52%.

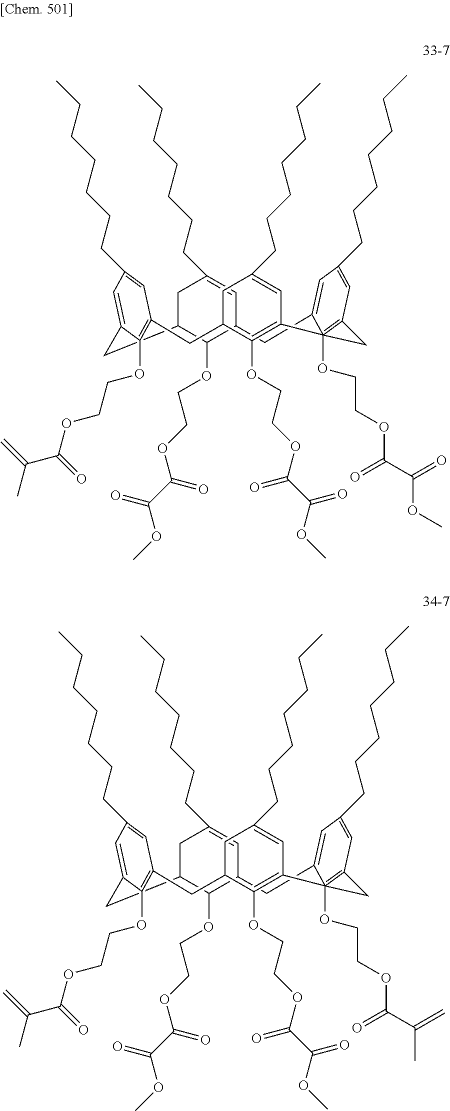

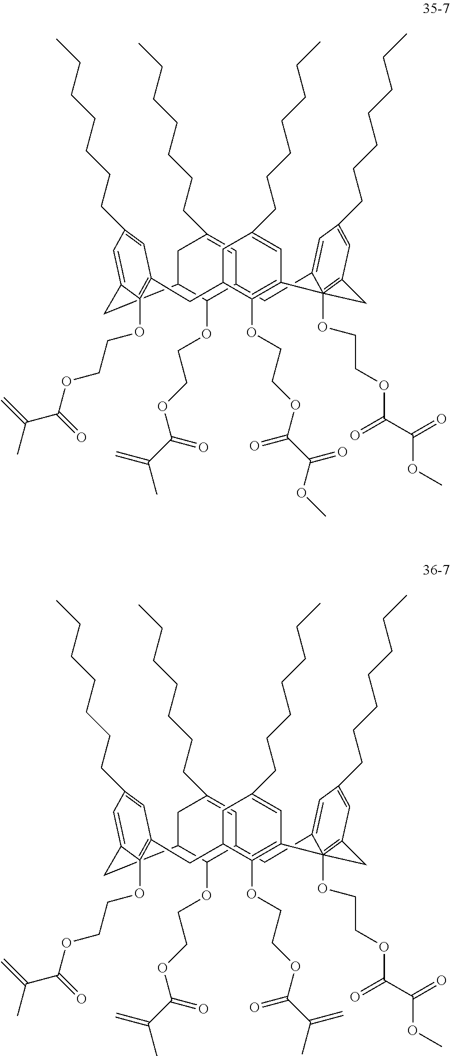

### Example 14

The same procedure as in Example 11 was repeated except that I-18 was used instead of I-6, to thereby obtain 0.433 g of the target compound 33-18 with a yield of 36.0%, 0.220 g of the target compound 34-18 with a yield of 18.5%, 0.221 g of the target compound 35-18 with a yield of 18.5%, and 0.112 g of the target compound 36-18 with a yield of 9.49%.

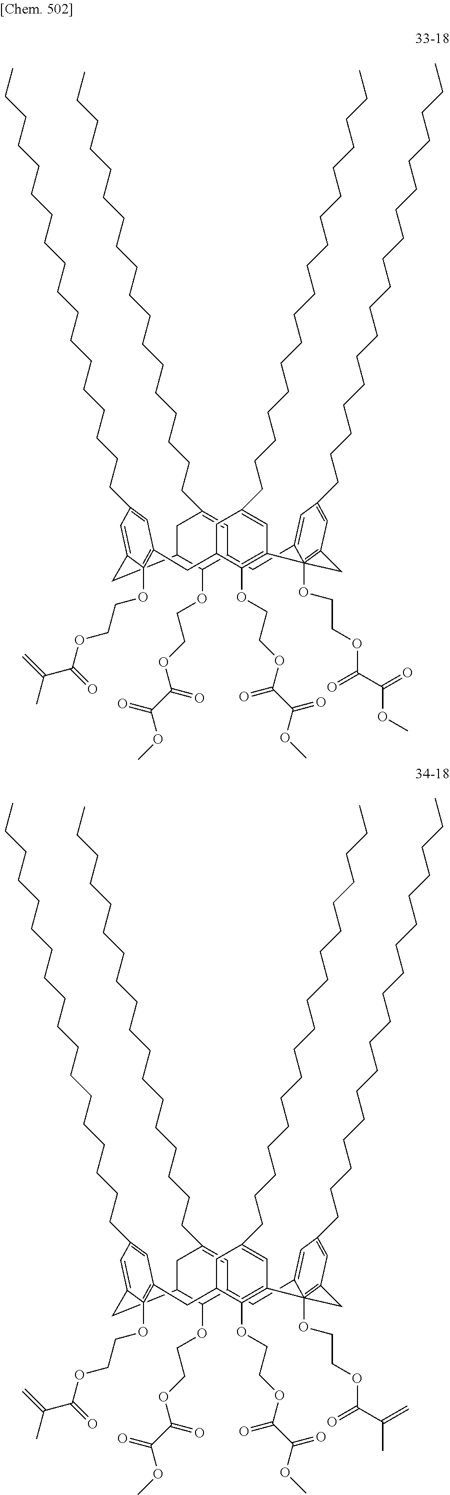

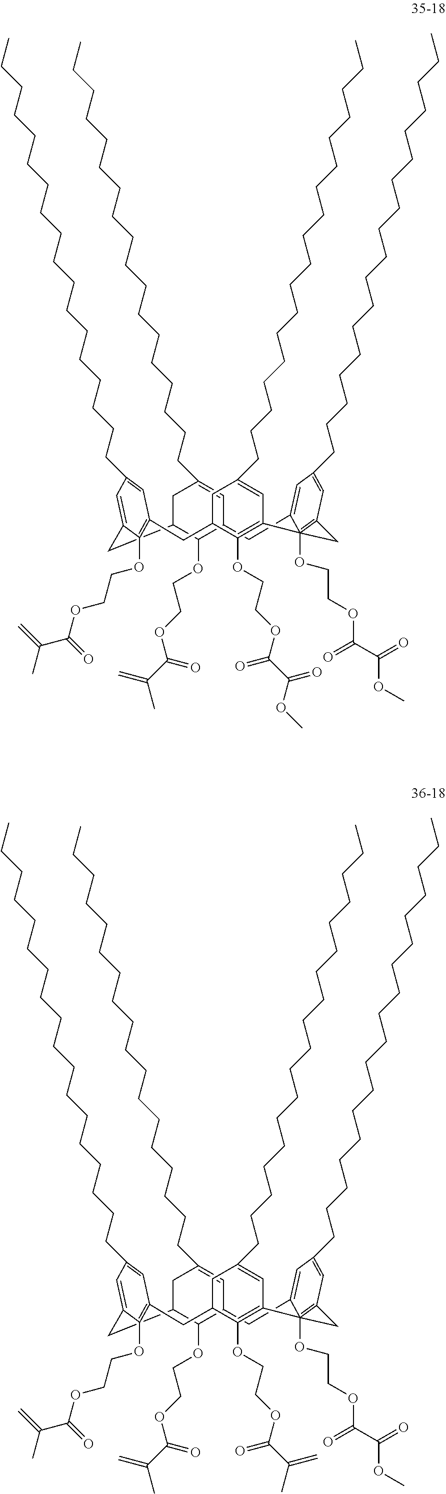

### Example 15

The same procedure as in Example 11 was repeated except that I-1 was used instead of I-6, to thereby obtain 0.367 g of the target compound 33-1 with a yield of 24.5%, 0.197 g of the target compound 34-1 with a yield of 13.5%, 0.187 g of the target compound 35-1 with a yield of 12.7%, and 0.101 g of the target compound 36-1 with a yield of 7.00%.

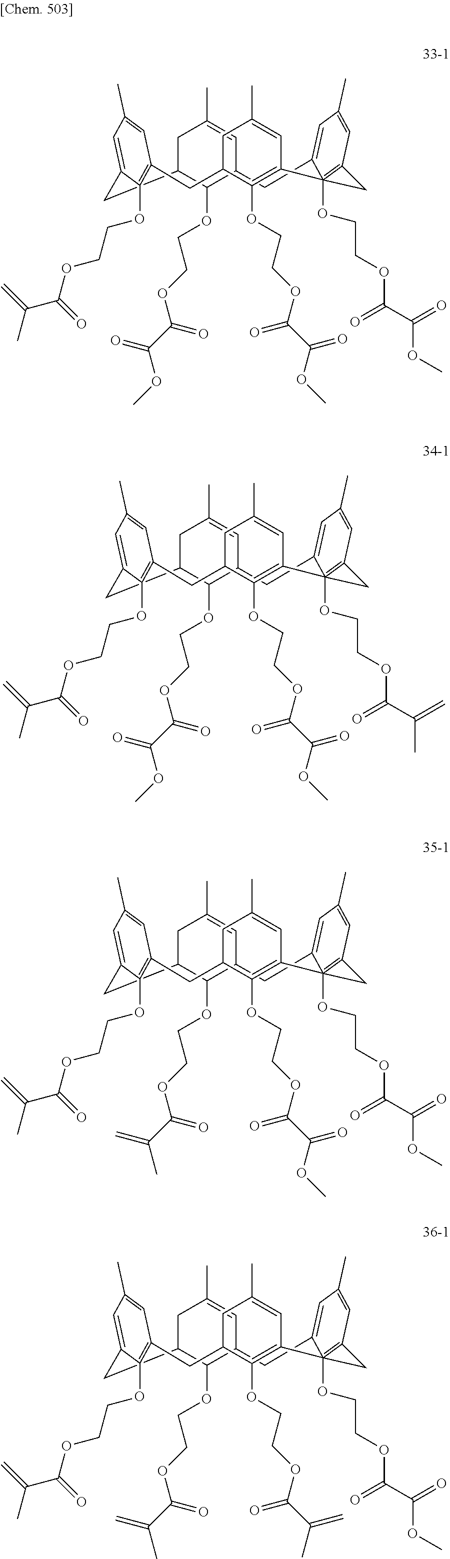

### Example 16

The same procedure as in Example 11 was repeated except that acrylic acid was used instead of methacrylic acid, to thereby obtain 0.401 g of the target compound 37-6 with a yield of 30.1%, 0.218 g of the target compound 38-6 with a yield of 16.8%, 0.21 g of the target compound 39-6 with a yield of 17.0%, and 0.111 g of the target compound 40-6 with a yield of 8.78%.

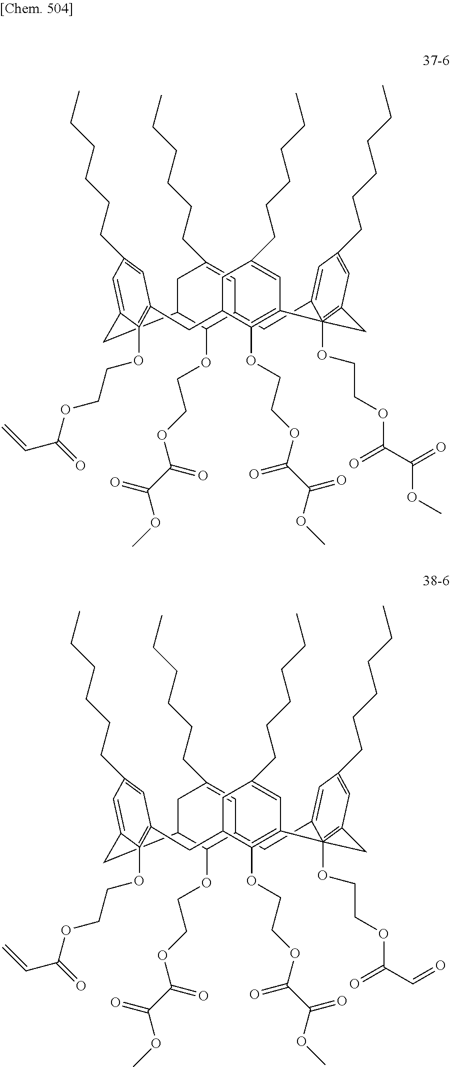

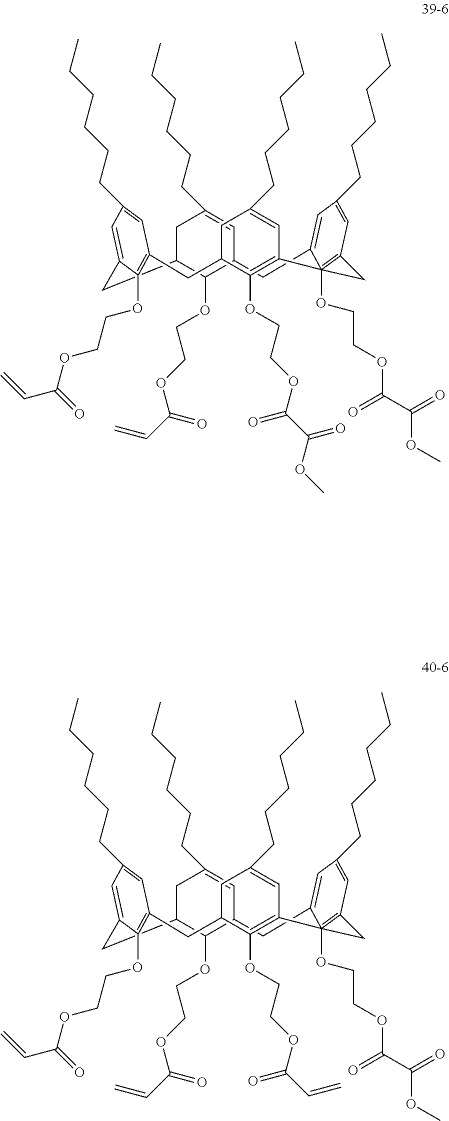

### Example 17

The same procedure as in Example 11 was repeated except that ethyl oxalyl chloride was used instead of methyl oxal chloride, to thereby obtain 0.404 g of the target compound 41-6 with a yield of 29.0%, 0.231 g of the target compound 42-6 with a yield of 17.0%, 0.228 g of the target compound 43-6 with a yield of 16.8%, and 0.124 g of the target compound 44-6 with a yield of 9.36%.

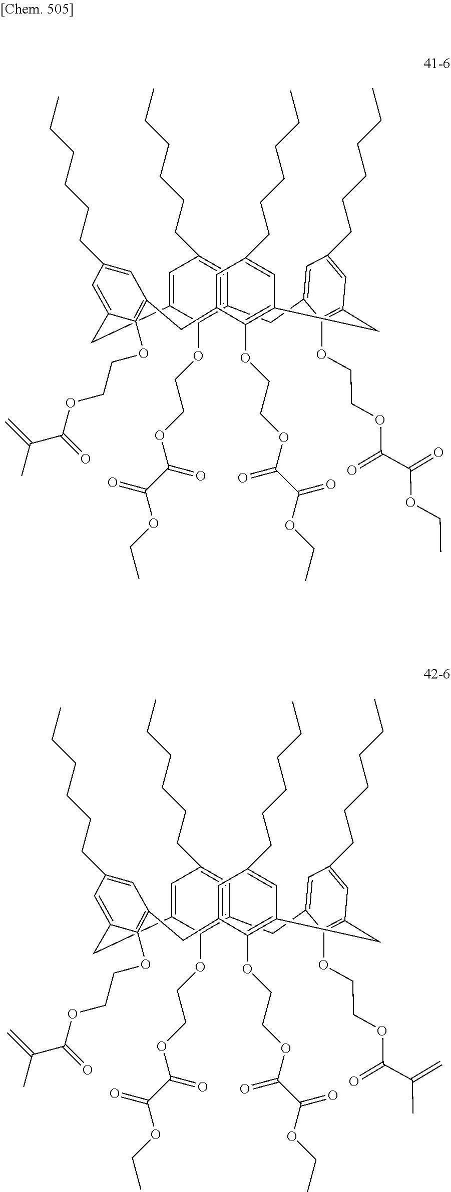

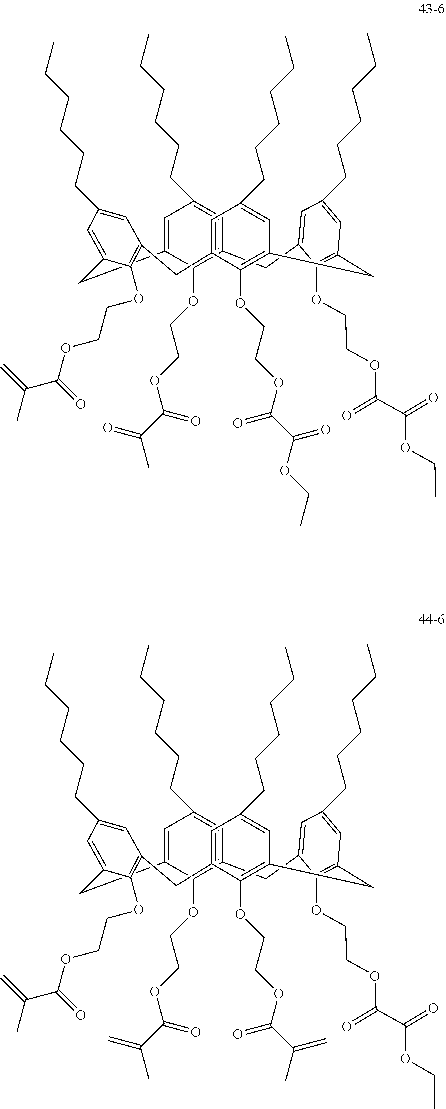

### Example 18

The same procedure as in Example 17 was repeated except that ethyl oxalyl chloride was used instead of methyl oxal chloride, to thereby obtain 0.389 g of the target compound 45-6 with a yield of 28.2%, 0.214 g of the target compound 46-6 with a yield of 16.1%, 0.212 g of the target compound 47-6 with a yield of 16.0%, and 0.111 g of the target compound 48-6 with a yield of 8.67%.

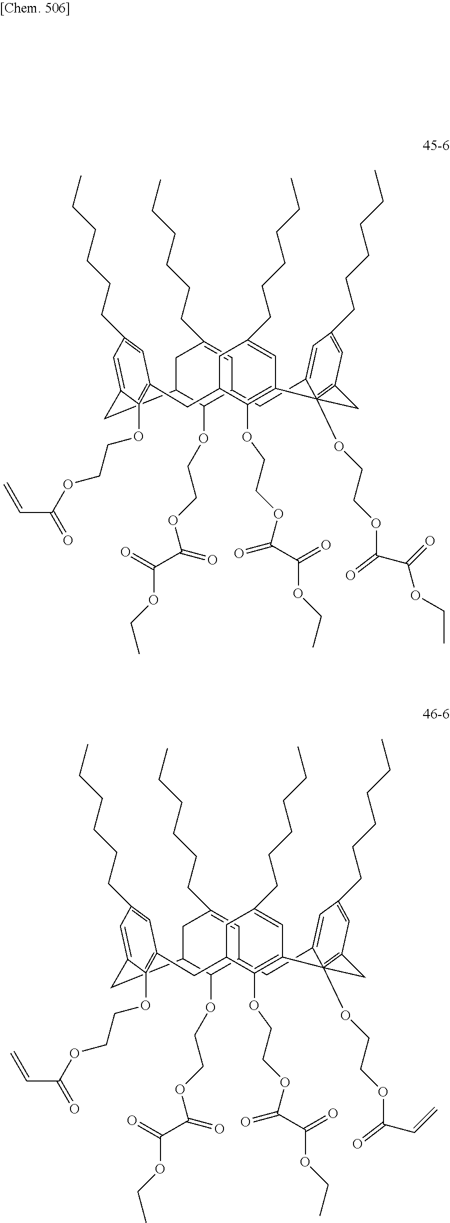

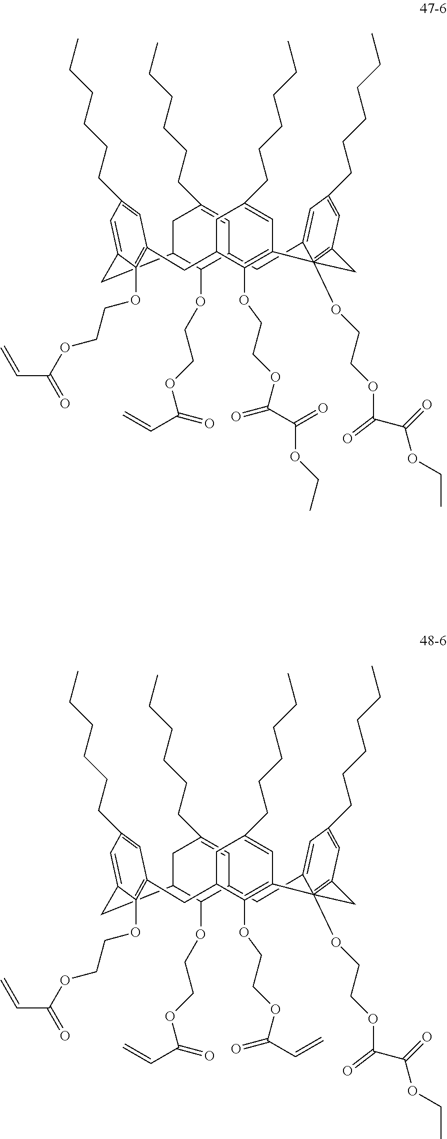

### Synthesis Example 30

The same procedure as in Synthesis Example 20 was repeated except that methyl bromopyopionate was used instead of methyl bromoacetate, to thereby obtain 4.89 g of compound J-6 represented by the following structural formula. The yield was 67.3%.

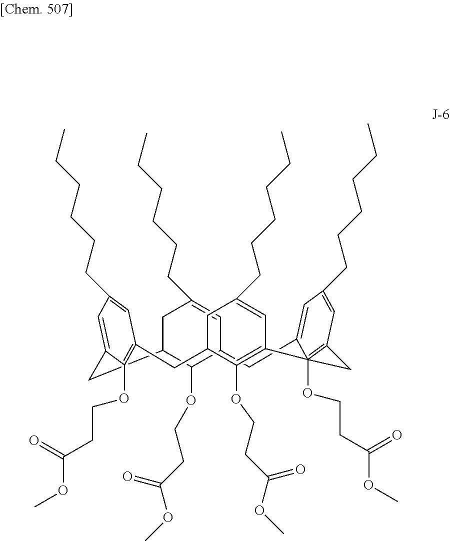

### Synthesis Example 31

The same procedure as in Synthesis Example 10 was repeated except that J-6 was used instead of C-6, to thereby obtain 3.88 g of compound K-6 represented by the following structural formula. The yield was 88.3%.

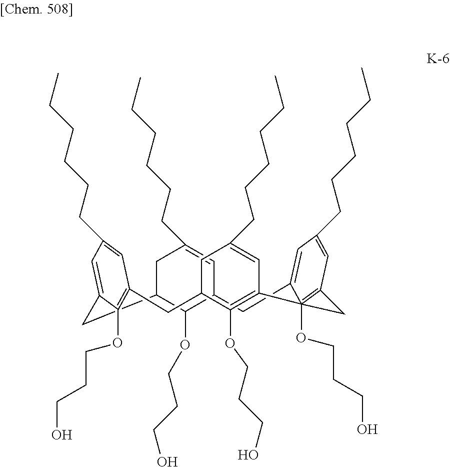

### Example 19

The same procedure as in Example 1 was repeated except that K-6 was used instead of D-6, to thereby obtain 0.412 g of the target compound 49-6 with a yield of 29.3%, 0.222 g of the target compound 50-6 with a yield of 16.0%, 0.219 g of the target compound 51-6 with a yield of 15.8%, and 0.135 g of the target compound 52-6 with a yield of 9.86%.

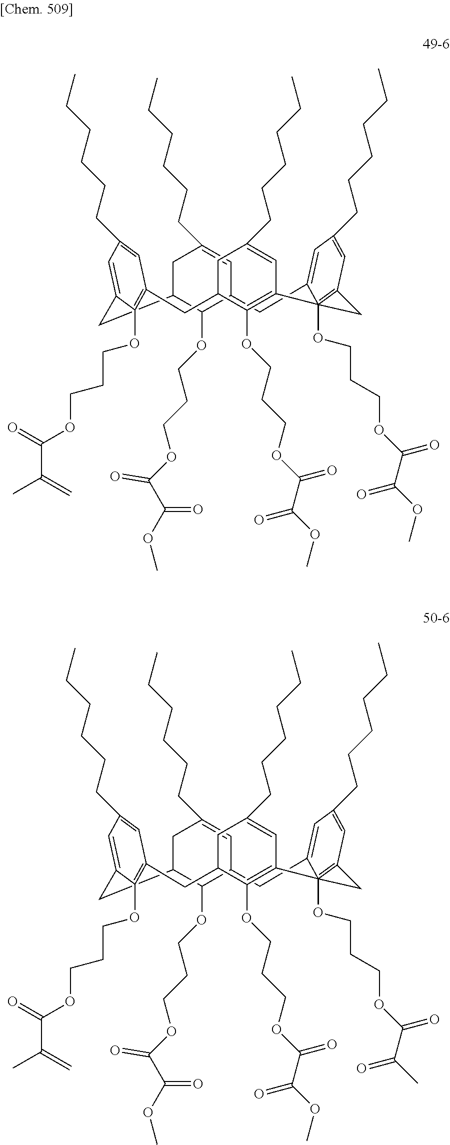

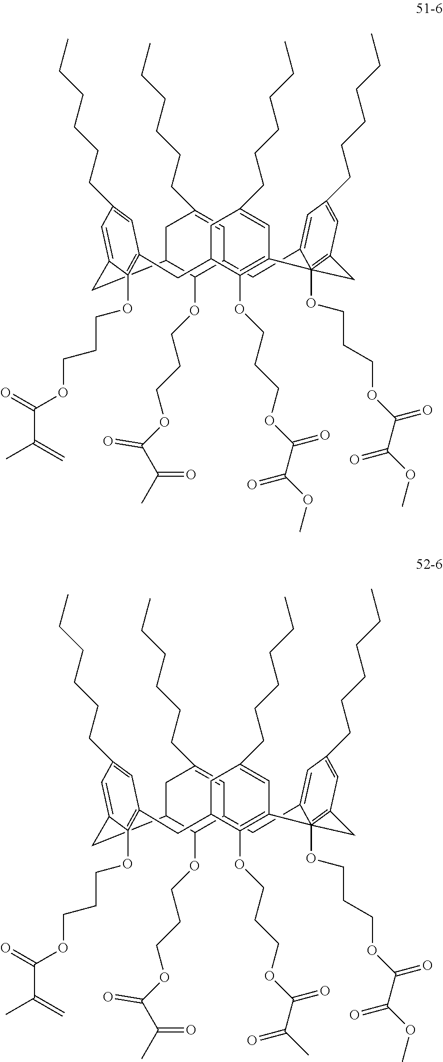

### Example 20

The same procedure as in Example 19 was repeated except that acrylic acid was used instead of methacrylic acid, to thereby obtain 0.399 g of the target compound 53-6 with a yield of 28.6%, 0.218 g of the target compound 54-6 with a yield of 15.9%, 0.208 g of the target compound 55-6 with a yield of 15.1%, and 0.117 g of the target compound 56-6 with a yield of 8.83%.

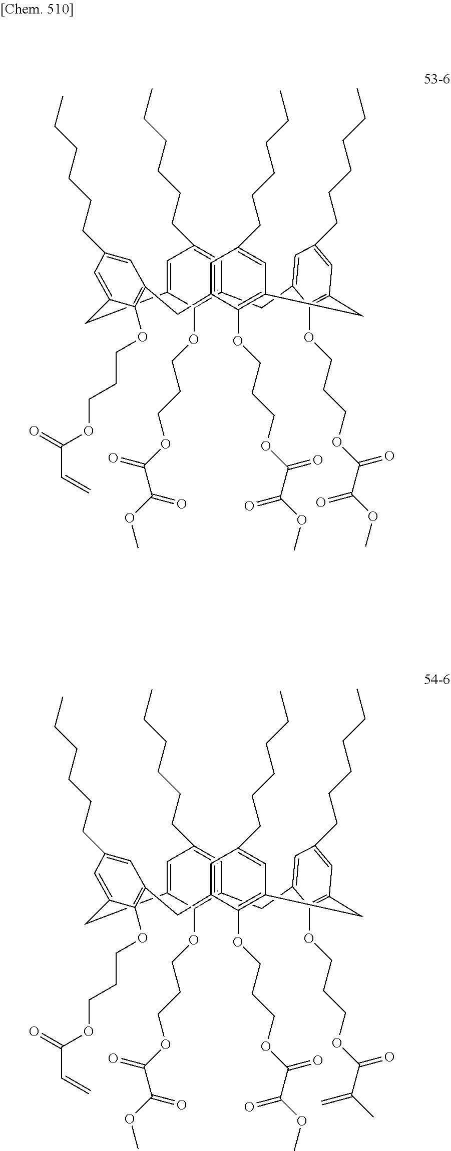 [SMILES: C=CC(=O)OCCCOc1c2cc(CCCCCC)cc1Cc1cc(CCCCCC)cc(c1OCCCOC(=O)C(=O)OC)Cc1cc(CCCCCC)cc(c1OCCCOC(=O)C(=O)OC)Cc1cc(CCCCCC)cc(c1OCCCOC(=O)C(=O)OC)C2 ; C=CC(=O)OCCCOc1c2cc(CCCCCC)cc1Cc1cc(CCCCCC)cc(c1OCCCOC(=O)C(=O)OC)Cc1cc(CCCCCC)cc(c1OCCCOC(=O)C(=C)C)Cc1cc(CCCCCC)cc(c1OCCCOC(=O)C(=O)OC)C2]

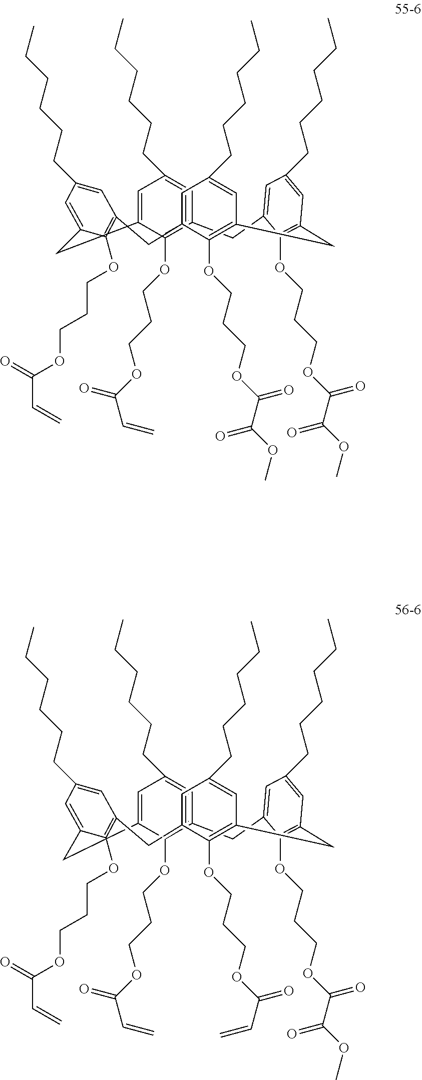 [SMILES: C=CC(=O)OCCCOc1c2cc(CCCCCC)cc1Cc1cc(CCCCCC)cc(c1OCCCOC(=O)C(=O)OC)Cc1cc(CCCCCC)cc(c1OCCCOC(=O)C(=O)OC)Cc1cc(CCCCCC)cc(c1OCCCOC(=O)C=C)C2 ; C=CC(=O)OCCCOc1c2cc(CCCCCC)cc1Cc1cc(CCCCCC)cc(c1OCCCOC(=O)C=C)Cc1cc(CCCCCC)cc(c1OCCCOC(=O)C(=O)OC)Cc1cc(CCCCCC)cc(c1OCCCOC(=O)C=C)C2]

### Example 21

The same procedure as in Example 19 was repeated except that ethyl oxalyl chloride was used instead of methyl oxal chloride, to thereby obtain 0.407 g of the target compound 57-6 with a yield of 29.7%, 0.201 g of the target compound 58-6 with a yield of 15.0%, 0.197 g of the target compound 59-6 with a yield of 14.7%, and 0.121 g of the target compound 60-6 with a yield of 9.26%.

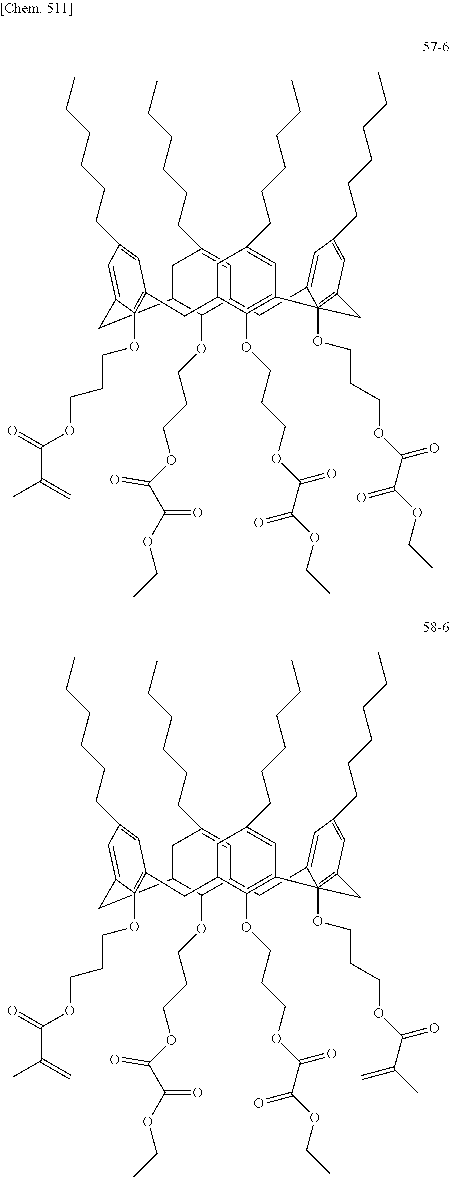

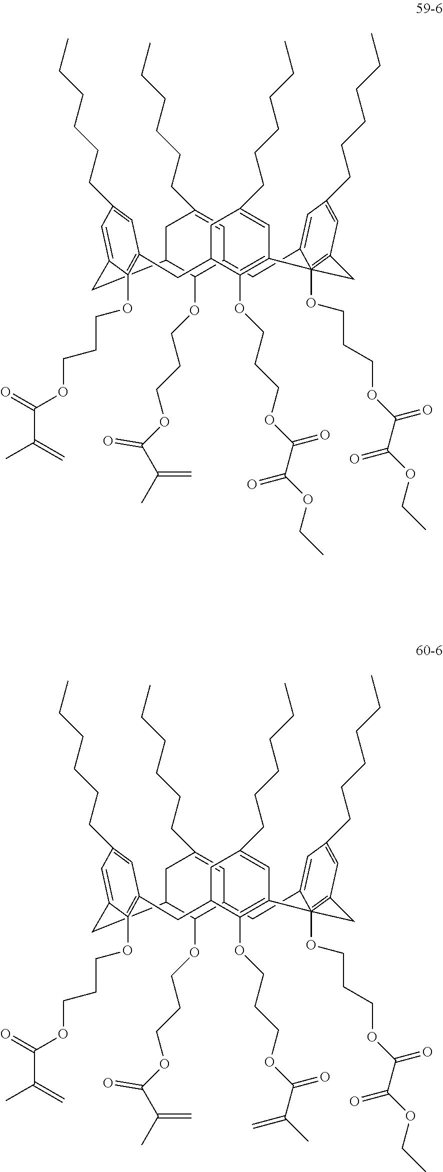

### Example 22

The same procedure as in Example 21 was repeated except that acrylic acid was used instead of methacrylic acid, to thereby obtain 0.395 g of the target compound 61-6 with a yield of 29.1%, 0.195 g of the target compound 62-6 with a yield of 14.9%, 0.184 g of the target compound 63-6 with a yield of 14.0%, and 0.102 g of the target compound 64-6 with a yield of 8.07%.

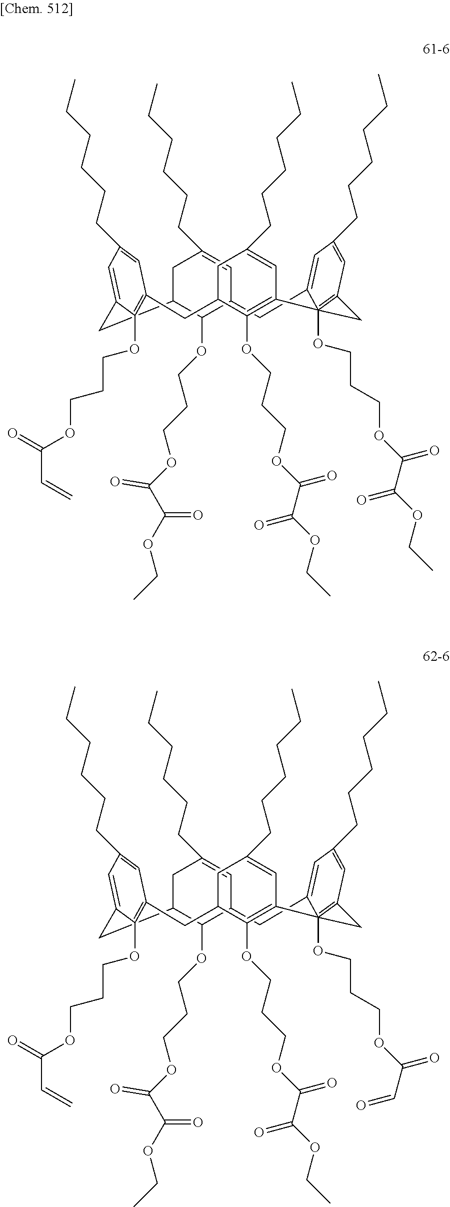

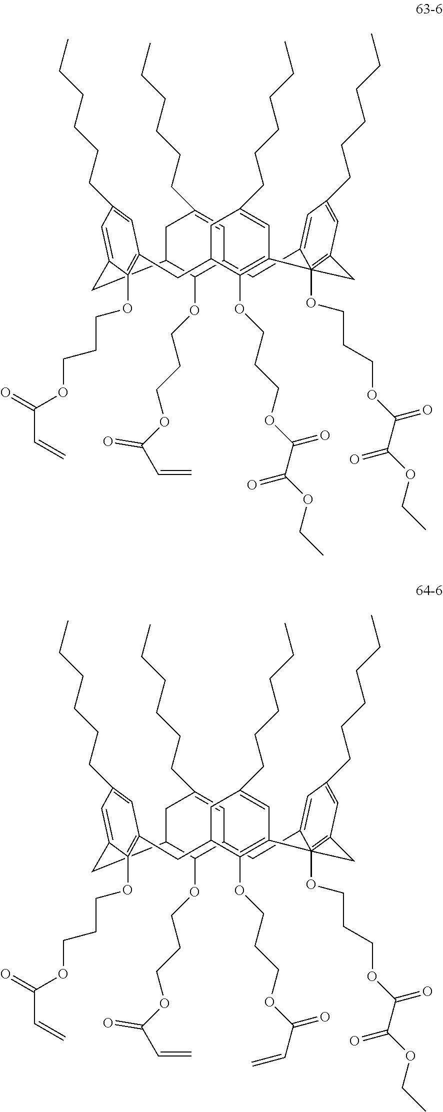

### Synthesis Example 32

A 50 mL four-necked flask equipped with a stirrer, a thermometer, and a reflux condenser tube was charged with 2.00 g (2.424 mmol) of I-6, 10.00 g of tetrahydrofuran, 1.2716 g (4.848 mmol) of triphenylphosphine, and 1.024 g (4.732 mmol) of 2-[[[(1,1-dimethylethyl)dimethylsilyl]oxyl-2-propenoic acid, and the mixture was stirred. A light yellow transparent solution was obtained. Then 0.9803 g (4.848 mmol) of diisopropyl azodicarboxylate was added dropwise to the mixture placed in an ice bath over 30 minutes. The light yellow transparent solution remained unchanged. The solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:acetone=95:5) to thereby obtain a light yellow transparent liquid. The solvent was concentrated, and chloroform/methanol were added to reprecipitate crystals. The white crystals were filtered on a Kiriyama funnel, and the obtained white crystals were vacuum dried (at 60° C. for 6 hours or longer) to thereby obtain 1.891 g of compound M-6 represented by the following structural formula. The yield was 48.2%.

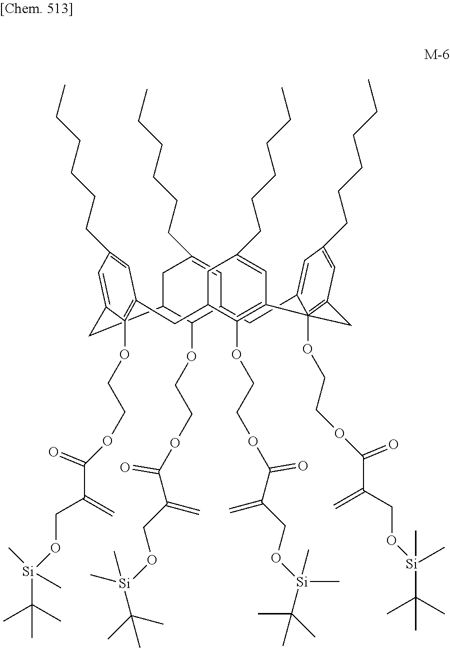

### Synthesis Example 33

The same procedure as in Synthesis Example 32 was repeated except that I-4 was used instead of I-6, to thereby obtain 1.641 g of compound M-4 represented by the following structural formula. The yield was 57.3%.

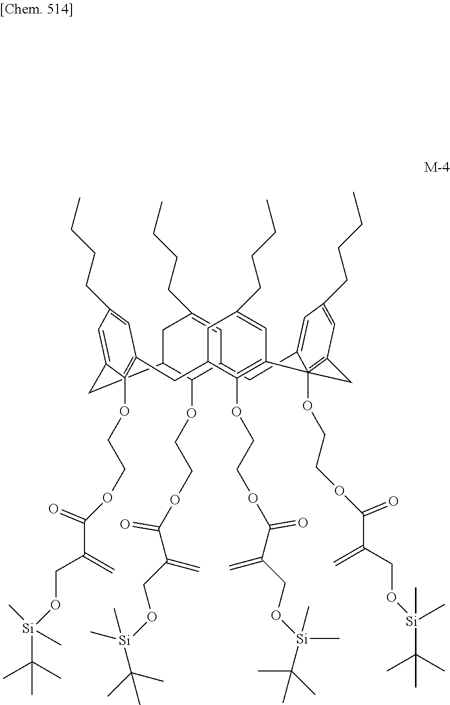

### Synthesis Example 34

The same procedure as in Synthesis Example 32 was repeated except that I-7 was used instead of I-6, to thereby obtain 1.880 g of compound M-7 represented by the following structural formula. The yield was 79.0%.

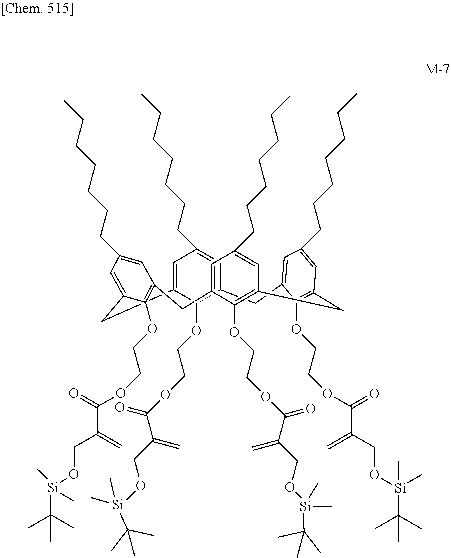 [SMILES: C=C(CO[Si](C)(C)C(C)(C)C)C(=O)OCCOc1c2cc(CCCCCCC)cc1Cc1cc(CCCCCCC)cc(c1OCCOC(=O)C(=C)CO[Si](C)(C)C(C)(C)C)Cc1cc(CCCCCCC)cc(c1OCCOC(=O)C(=C)CO[Si](C)(C)C(C)(C)C)Cc1cc(CCCCCCC)cc(c1OCCOC(=O)C(=C)CO[Si](C)(C)C(C)(C)C)C2]

### Synthesis Example 35

The same procedure as in Synthesis Example 32 was repeated except that I-18 was used instead of I-6, to thereby obtain 2.132 g of compound M-18 represented by the following structural formula. The yield was 71.4%.

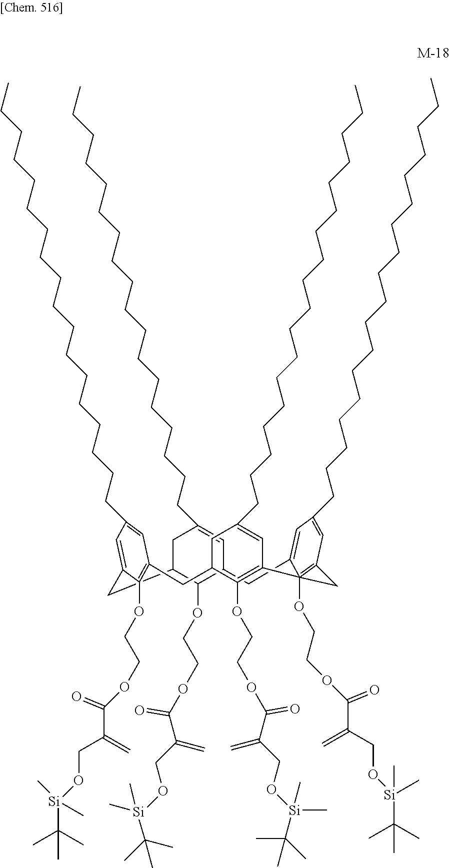

### Synthesis Example 36

The same procedure as in Synthesis Example 32 was repeated except that I-1 was used instead of I-6, to thereby obtain 1.762 g of compound M-1 represented by the following structural formula. The yield was 39.9%.

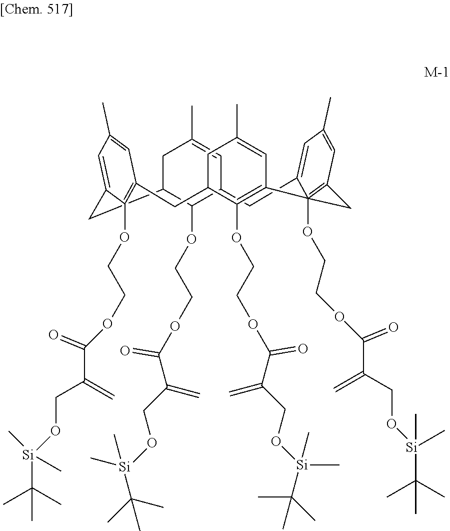

### Synthesis Example 37

A 100 mL four-necked flask equipped with a stirrer, a thermometer, and a reflux condenser tube was charged with 1.891 g (1.168 mmol) of M-6, 50.00 g of tetrahydrofuran, and 0.3367 g (5.606 mmol) acetic acid, and the mixture was stirred. Then tetrabutylammonium fluoride (an about 1 mol/L tetrahydrofuran solution; 5.61 mL (5.61 mmol)) was slowly added to the mixture placed in an ice bath under stirring. The resulting light yellow transparent reaction solution was stirred at room temperature for 6 hours. Ion exchanged water was added to the resulting solution placed in an ice bath to stop the reaction, and then 30 g of chloroform was added. The reaction mixture was transferred to a separatory funnel, and the organic layer was separated. Next, the aqueous layer was extracted with 30 g of chloroform three times, and the extract was combined with the organic layer. The organic layer was pre-dried over anhydrous magnesium sulfate, and the solvent was removed by evaporation using an evaporator to thereby obtain a red transparent liquid. The red transparent liquid was purified by column chromatography (eluent: n-hexane:acetone=95:5), and chloroform/methanol were added to the resulting light yellow transparent liquid to reprecipitate crystals. The white crystals were filtered on a Kiriyama funnel and vacuum dried (at 60° C. for 6 hours or longer) to thereby obtain 0.8451 g of compound N-6 represented by the following structural formula. The yield was 62.3%.

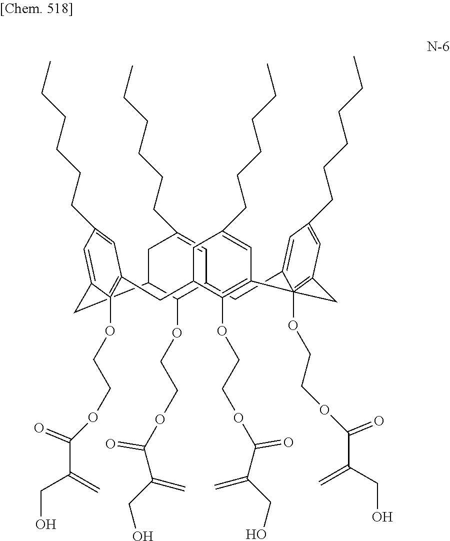

### Synthesis Example 38

The same procedure as in Synthesis Example 37 was repeated except that M-4 was used instead of M-6, to thereby obtain 0.639 g of compound N-4 represented by the following structural formula. The yield was 54.3%.

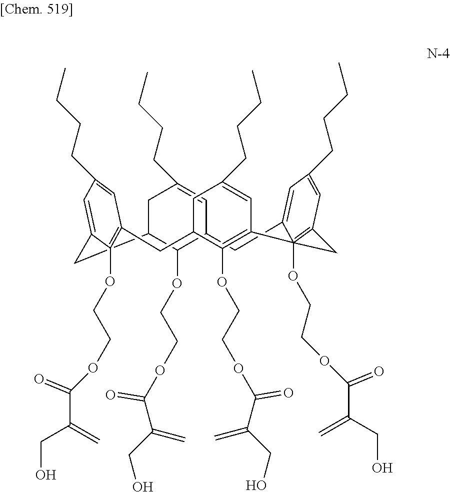

### Synthesis Example 39

The same procedure as in Synthesis Example 37 was repeated except that M-7 was used instead of M-6, to thereby obtain 0.873 g of compound N-7 represented by the following structural formula. The yield was 62.4%.

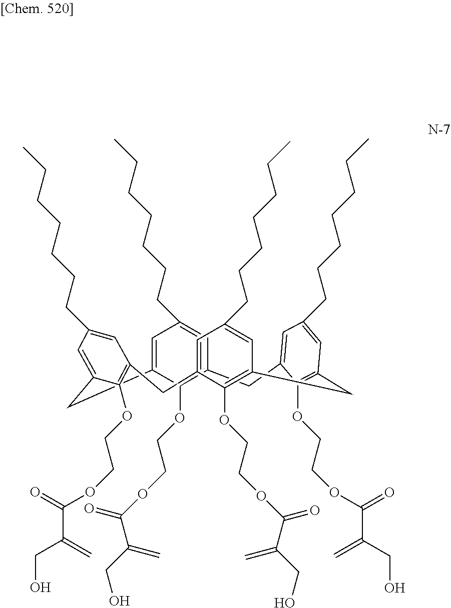 [SMILES: C=C(CO)C(=O)OCCOc1c2cc(CCCCCCC)cc1Cc1cc(CCCCCCC)cc(c1OCCOC(=O)C(=C)CO)Cc1cc(CCCCCCC)cc(c1OCCOC(=O)C(=C)CO)Cc1cc(CCCCCCC)cc(c1OCCOC(=O)C(=C)CO)C2]

### Synthesis Example 40

The same procedure as in Synthesis Example 37 was repeated except that M-18 was used instead of M-6, to thereby obtain 1.092 g of compound N-18 represented by the following structural formula. The yield was 63.2%.

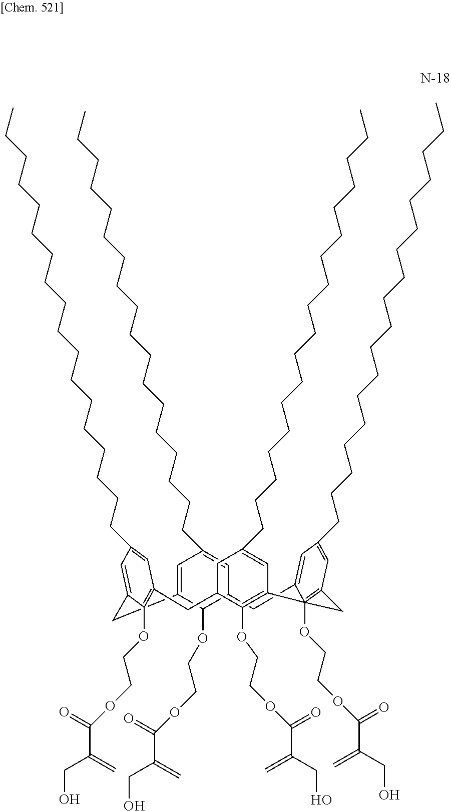

### Synthesis Example 41

The same procedure as in Synthesis Example 37 was repeated except that M-1 was used instead of M-6, to thereby obtain 0.654 g of compound N-1 represented by the following structural formula. The yield was 54.2%.

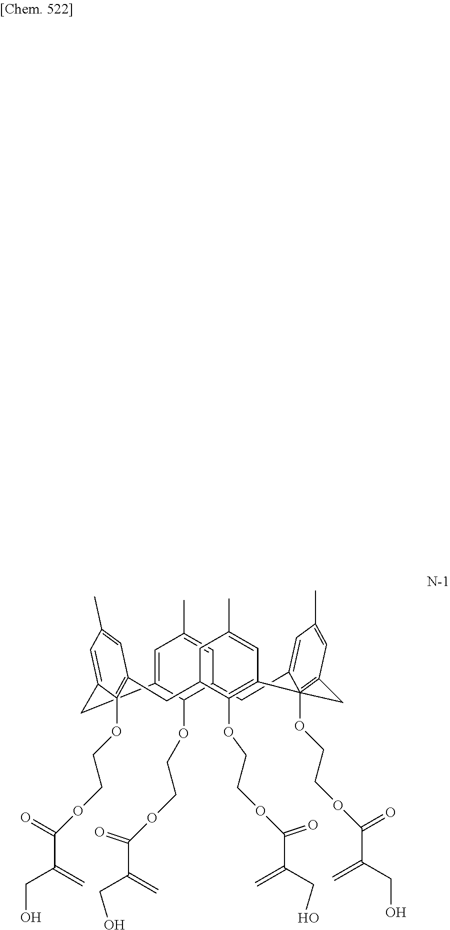

### Example 23

A 30 mL four-necked flask equipped with a stirrer, a thermometer, and a reflux condenser tube was charged with 0.300 g (0.236 mmol) of N-6, 0.679 g of tetrahydrofuran, and 0.74 g (6.04 mmol) of methyl oxal chloride, and the mixture was stirred under cooling with ice. 0.61 g (6.04 mmol) of triethylamine dissolved in 1.20 g of tetrahydrofuran was added to the resulting mixture, and the mixture was stirred at room temperature for 3 hours. Water was added to the reaction solution to stop the reaction, and the resulting solution was extracted with ethyl acetate, washed with water and saturated brine, and dried over magnesium sulfate. The solvent was removed by evaporation using an evaporator to thereby obtain a red viscous liquid. The red viscous liquid was purified by column chromatography (eluent: n-hexane:ethyl acetate=85:15) to thereby obtain 0.278 g of the target compound 65-6. The yield was 70.5%.

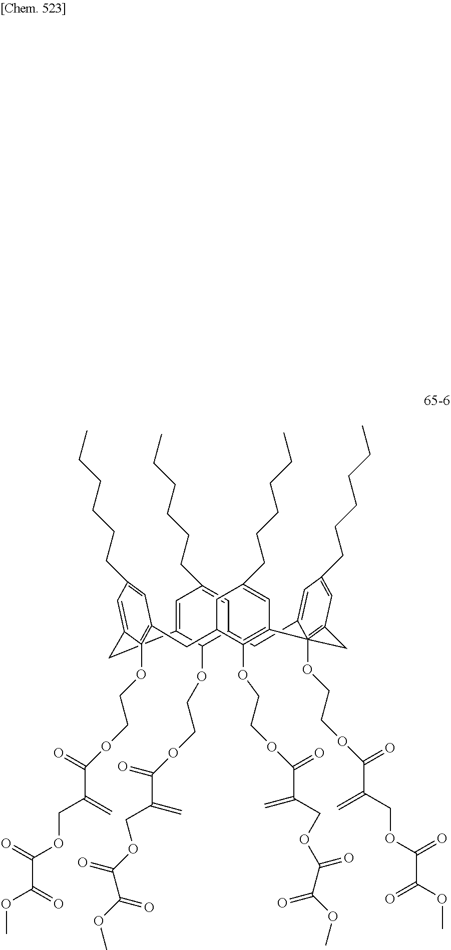

### Example 24

The same procedure as in Example 23 was repeated except that N-4 was used instead of N-6, to thereby obtain 0.281 g of the target compound 65-4. The yield was 72.3%.

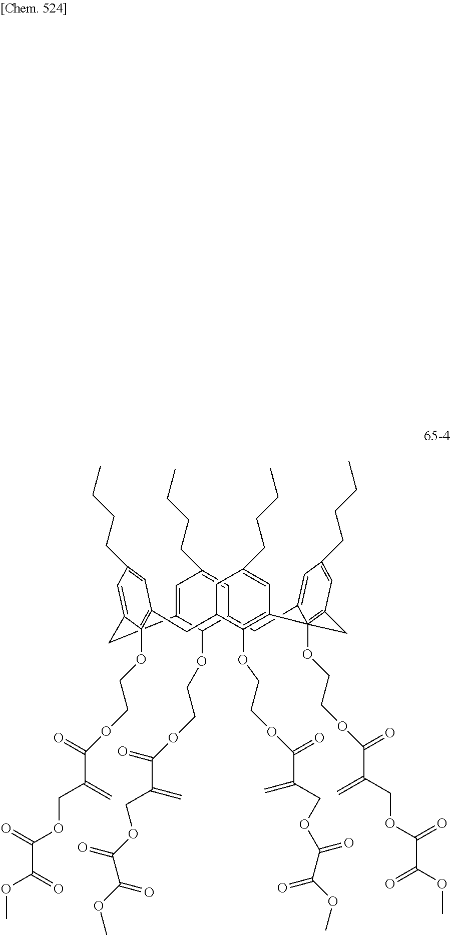

### Example 25

The same procedure as in Example 23 was repeated except that N-7 was used instead of N-6, to thereby obtain 0.301 g of the target compound 65-7. The yield was 79.7%.

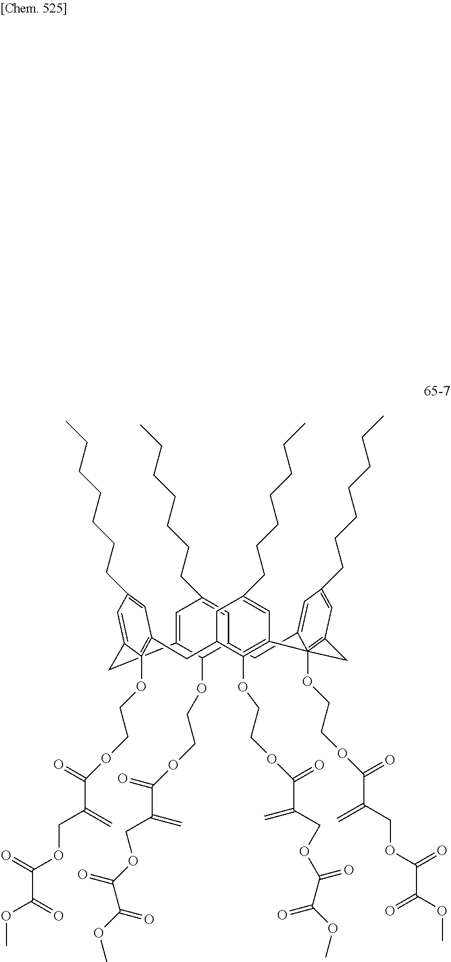

### Example 26

The same procedure as in Example 23 was repeated except that N-18 was used instead of N-6, to thereby obtain 0.297 g of the target compound 65-18. The yield was 84.1%.

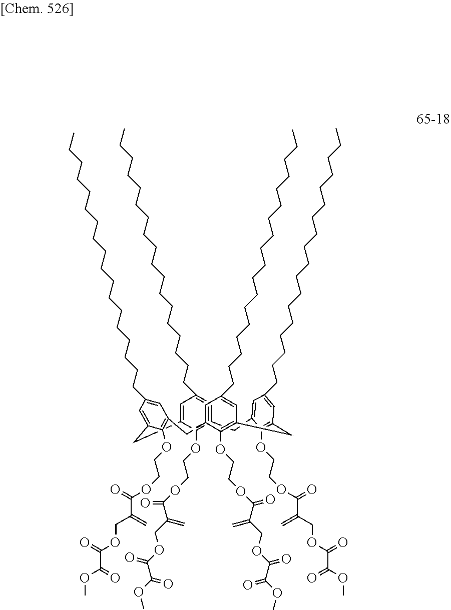 [SMILES: C=C(COC(=O)C(=O)OC)C(=O)OCCOc1c2cc(CCCCCCCCCCCCCCCCCC)cc1Cc1cc(CCCCCCCCCCCCCCCCCC)cc(c1OCCOC(=O)C(=C)COC(=O)C(=O)OC)Cc1cc(CCCCCCCCCCCCCCCCCC)cc(c1OCCOC(=O)C(=C)COC(=O)C(=O)OC)Cc1cc(CCCCCCCCCCCCCCCCCC)cc(c1OCCOC(=O)C(=C)COC(=O)C(=O)OC)C2]

### Example 27

The same procedure as in Example 23 was repeated except that N-1 was used instead of N-6, to thereby obtain 0.230 g of the target compound 65-1. The yield was 56.9%.

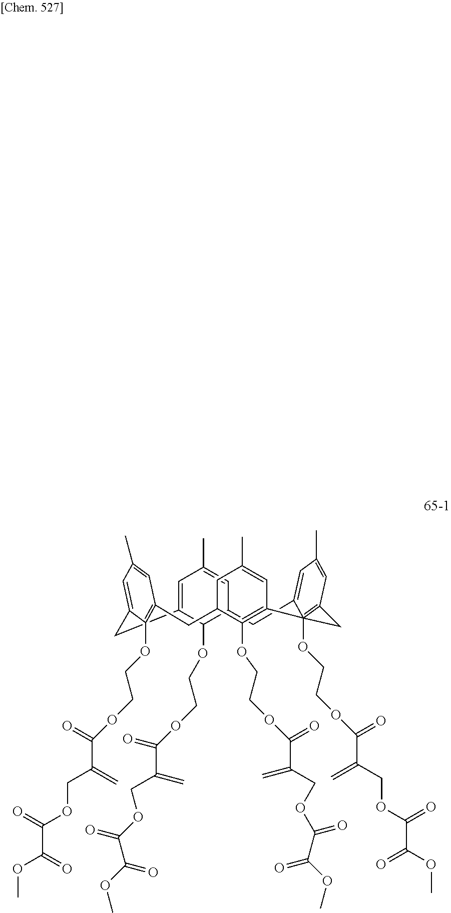

### Example 30

The same procedure as in Example 23 was repeated except that ethyl oxalyl chloride was used instead of methyl oxal chloride, to thereby obtain 0.303 g of the target compound 66-6. The yield was 75.1%.

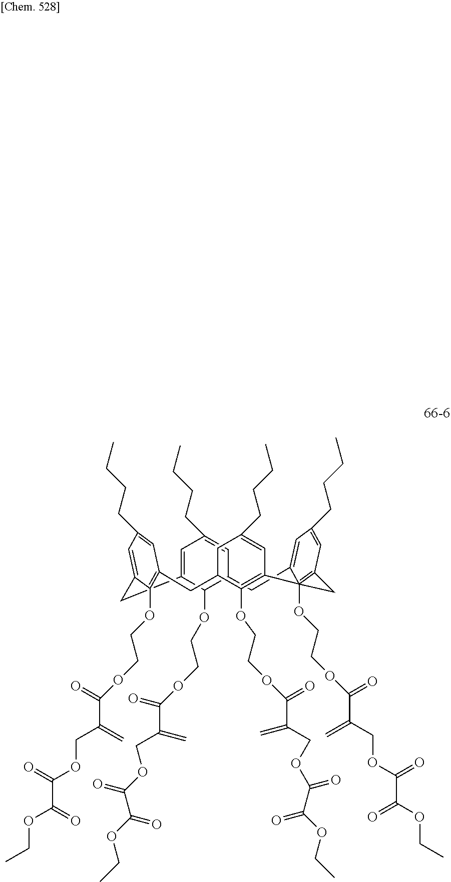

### Synthesis Example 42

The same procedure as in Synthesis Example 32 was repeated except that 4-[[[(1,1-dimethylethyl)dimethylsilyl]oxy]-2-methylenebutanoic acid was used instead of 2-[[[(1,1-dimethylethyl)dimethylsilyl]oxy]2-propenoic acid, to thereby obtain 2.420 g of compound O-6 represented by the following structural formula. The yield was 72.6%.

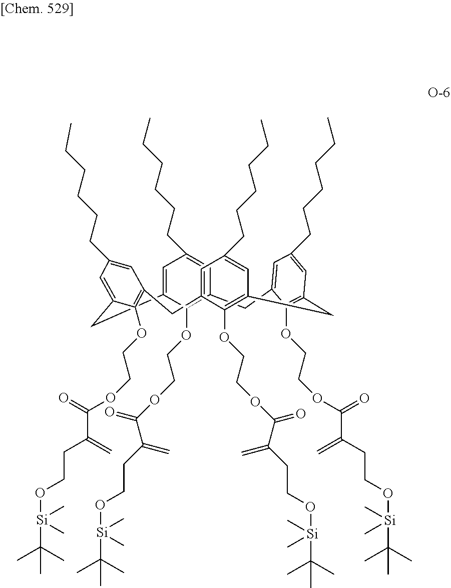 [SMILES: C=C(CCO[Si](C)(C)C(C)(C)C)C(=O)OCCOc1c2cc(CCCCCC)cc1Cc1cc(CCCCCC)cc(c1OCCOC(=O)C(=C)CCO[Si](C)(C)C(C)(C)C)Cc1cc(CCCCCC)cc(c1OCCOC(=O)C(=C)CCO[Si](C)(C)C(C)(C)C)Cc1cc(CCCCCC)cc(c1OCCOC(=O)C(=C)CCO[Si](C)(C)C(C)(C)C)C2]

### Synthesis Example 43

The same procedure as in Synthesis Example 37 was repeated except that O-6 was used instead of M-6, to thereby obtain 1.07 g of compound P-6 represented by the following structural formula. The yield was 59.4%.

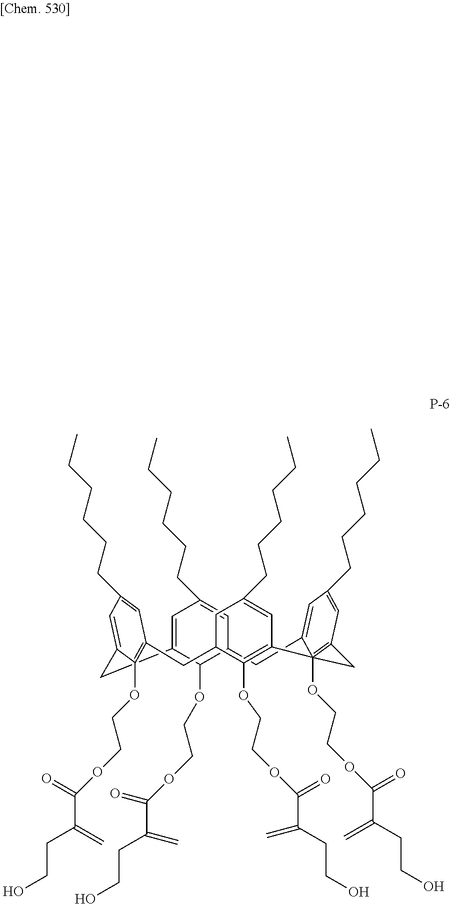

### Example 29

The same procedure as in Example 23 was repeated except that P-6 was used instead of N-6, to thereby obtain 0.287 g of the target compound 67-6. The yield was 76.0%.

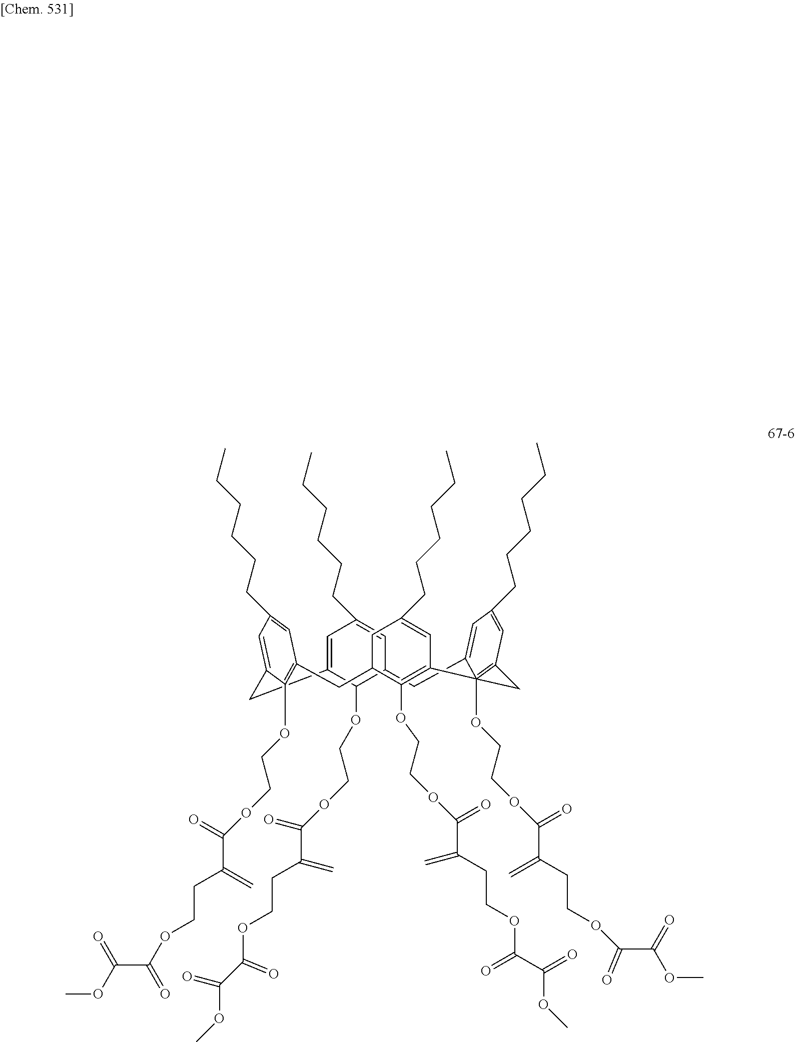

### Example 30

The same procedure as in Example 29 was repeated except that ethyl oxalyl chloride was used instead of methyl oxal chloride, to thereby obtain 0.266 g of the target compound 68-6. The yield was 68.2%.

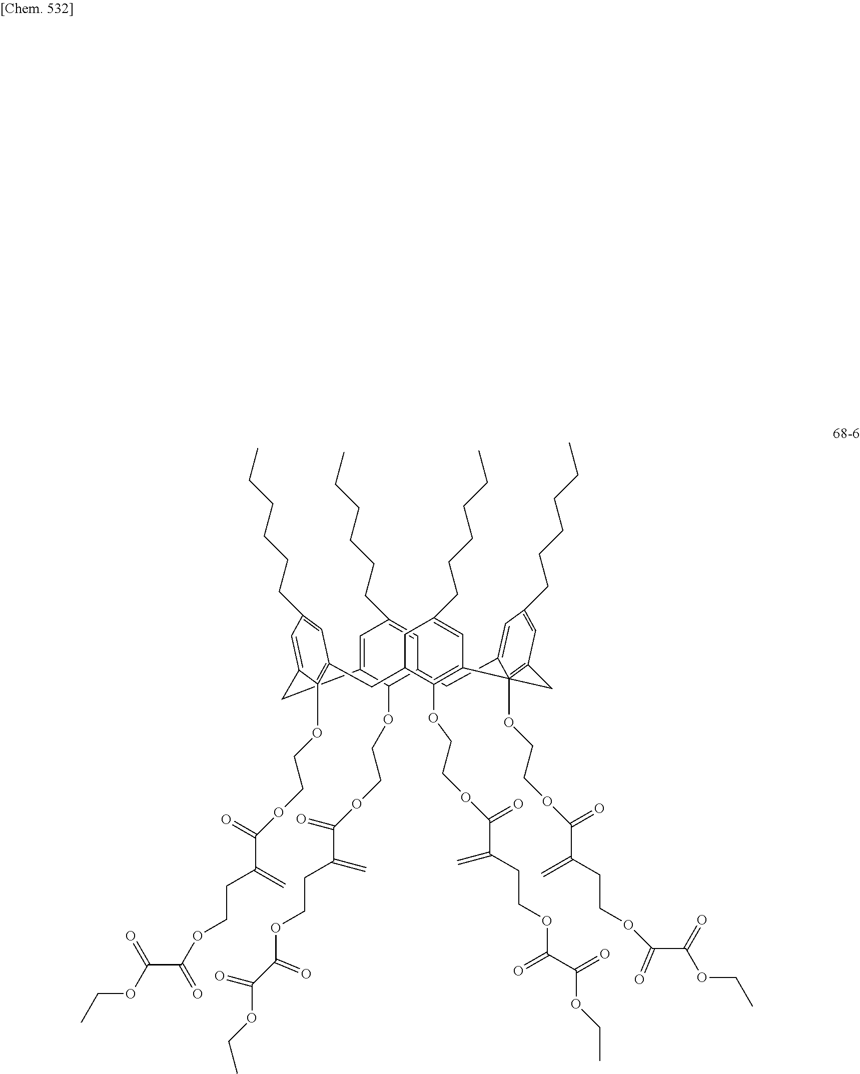

### Synthesis Example 44

A 1 L four-necked flask equipped with a stirrer, a dropping funnel, a thermometer, and a reflux condenser tube was charge with, in a nitrogen atmosphere, sodium hydride (7.54 g, 188.4 mmol), and mineral oil was removed by washing with hexane. Then dry DMF (160 mL) and hexyl bromide (37.2 g, 207.4 mmol) were added, and the resulting mixture was heated to 70° C. under stirring. A solution prepared by dissolving intermediate A (10 g, 23.6 mmol) obtained in Synthesis Example 1 in dry DMF (80 mL) was added to the mixture from a dropping funnel. After completion of the addition, the resulting mixture was continuously stirred for additional 2 hours. After cooled to room temperature, the reaction mixture was poured onto ice (300 g), and concentrated hydrochloric acid was added to acidify the aqueous solution. Then the solution was extracted with chloroform (200 mL) twice. The chloroform solution was washed with water until the pH was 5 or higher, further washed with saturated brine, and dried over anhydrous magnesium sulfate. The solvent was removed using an evaporator to thereby obtain a yellow liquid. Methanol was added to the mixture under stirring to precipitate solids. The solids were collected by filtration and recrystallized with isopropyl alcohol. The obtained white crystals were vacuum dried to thereby obtain a compound represented by the following formula (11.6 g, yield 65%).

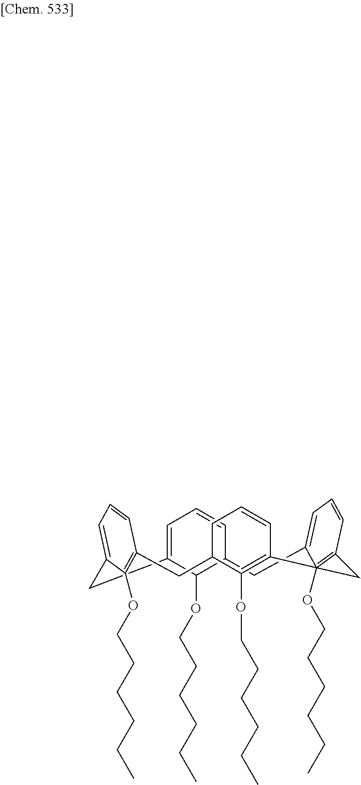

### Synthesis Example 45

The same procedure as in Synthesis Example 44 was repeated except that methyl iodide was used instead of hexyl bromide and that the reaction was performed at room temperature for 24 hours, to thereby obtain a compound represented by the following formula (6.8 g, yield 60%).

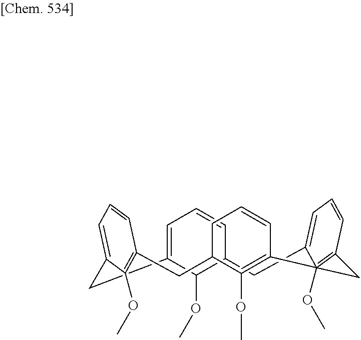

### Synthesis Example 46

The same procedure as in Synthesis Example 44 was repeated except that butyl bromide was used instead of hexyl bromide, to thereby obtain a compound represented by the following formula (11.0 g, yield 72%).

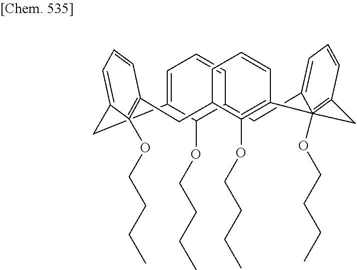

### Synthesis Example 47

The same procedure as in Synthesis Example 44 was repeated except that heptyl bromide was used instead of hexyl bromide, to thereby obtain a compound represented by the following formula (14.4 g, yield 75%).

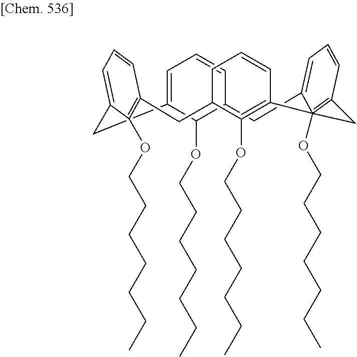

### Synthesis Example 48

The same procedure as in Synthesis Example 44 was repeated except that octadecyl bromide was used instead of hexyl bromide, to thereby obtain a compound represented by the following formula (23.6 g, yield 70%).

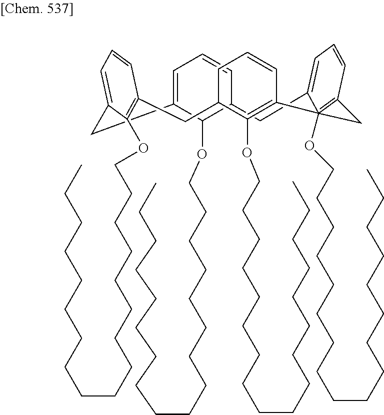

### Synthesis Example 49

Referring to a known document (Organic & Biomolecular Chemistry, 13, 1708-1723; 2015), a compound represented by the following formula was synthesized in two steps using the compound obtained in Synthesis Example 44 (5.0 g, 6.57 mmol) (yield amount 3.3 g, yield 67%).

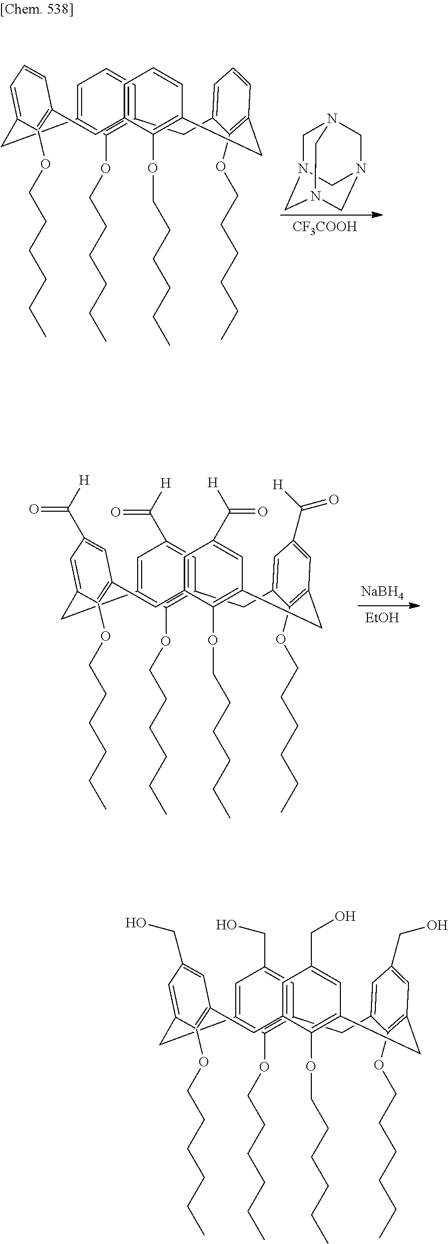

### Synthesis Example 50

The same procedure as in Synthesis Example 49 was repeated except that the compound obtained in Synthesis Example 45 (5.0 g, 10.4 mmol) was used instead of the compound obtained in Synthesis Example 44, to synthesize a compound represented by the following formula in two steps (3.75 g, yield 60%).

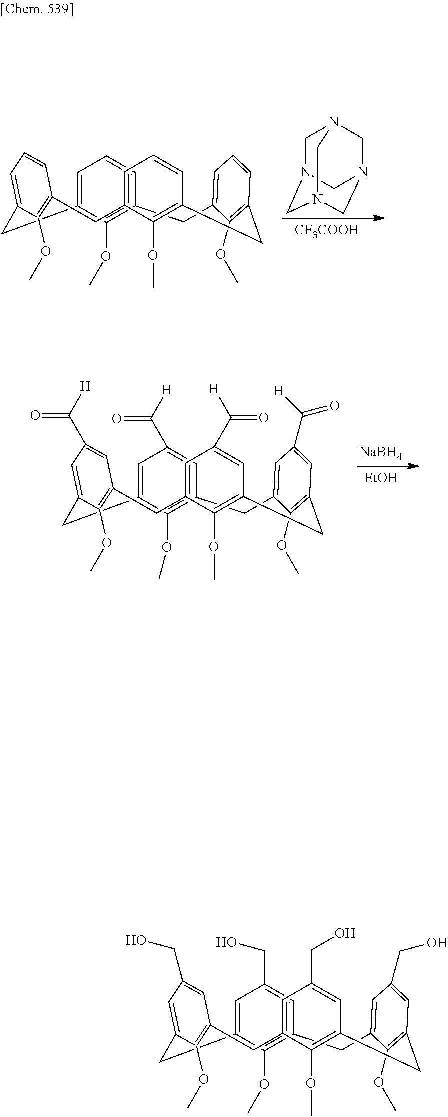

### Synthesis Example 51

The same procedure as in Synthesis Example 49 was repeated except that the compound obtained in Synthesis Example 46 (5.0 g, 7.7 mmol) was used instead of the compound obtained in Synthesis Example 44, to synthesize a compound represented by the following formula in two steps (3.73 g, yield 63%).

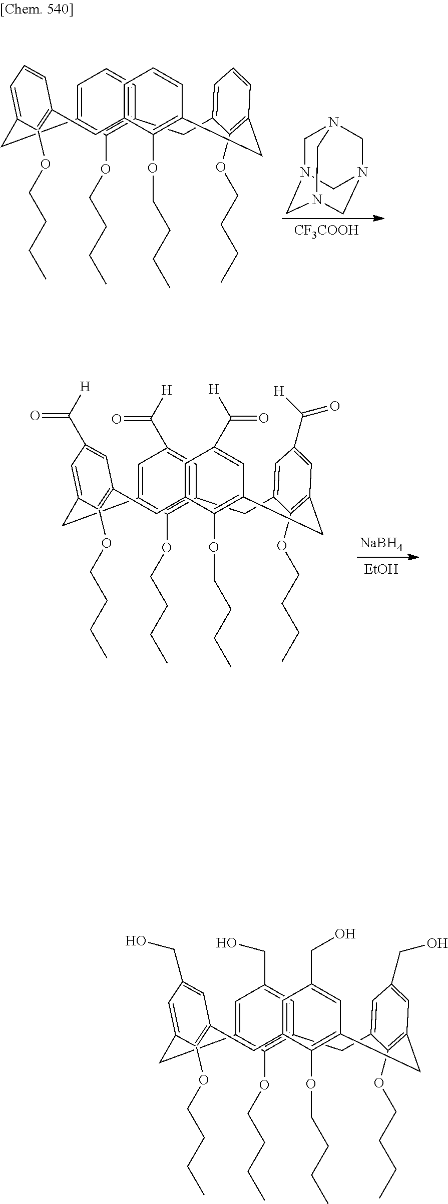

### Synthesis Example 52

The same procedure as in Synthesis Example 49 was repeated except that the compound obtained in Synthesis Example 47 (5.0 g, 6.1 mmol) was used instead of the compound obtained in Synthesis Example 44, to synthesize a compound represented by the following formula in two steps (4.01 g, yield 70%).

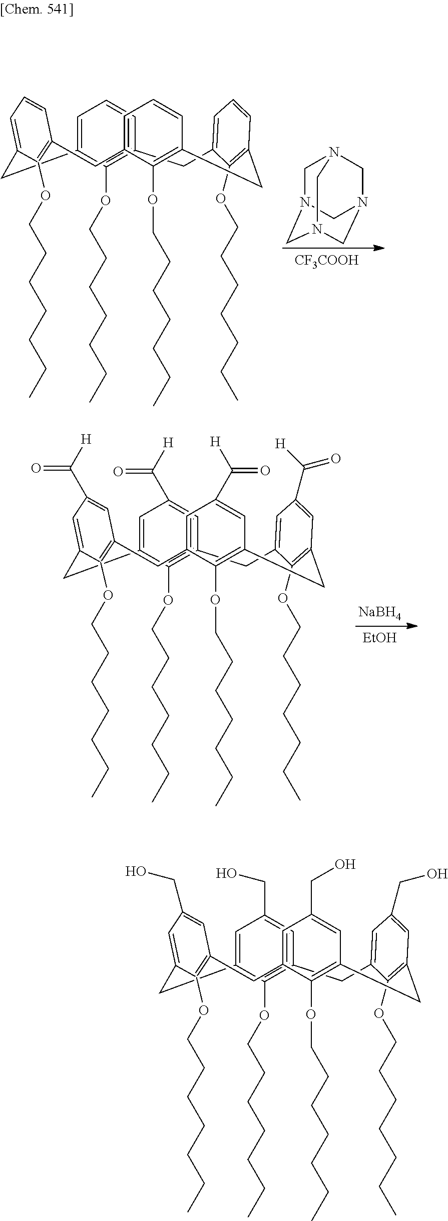

### Synthesis Example 53

The same procedure as in Synthesis Example 49 was repeated except that the compound obtained in Synthesis Example 48 (10.0 g, 7.0 mmol) was used instead of the compound obtained in Synthesis Example 44, to synthesize a compound represented by the following formula in two steps (5.96 g, yield 55%).

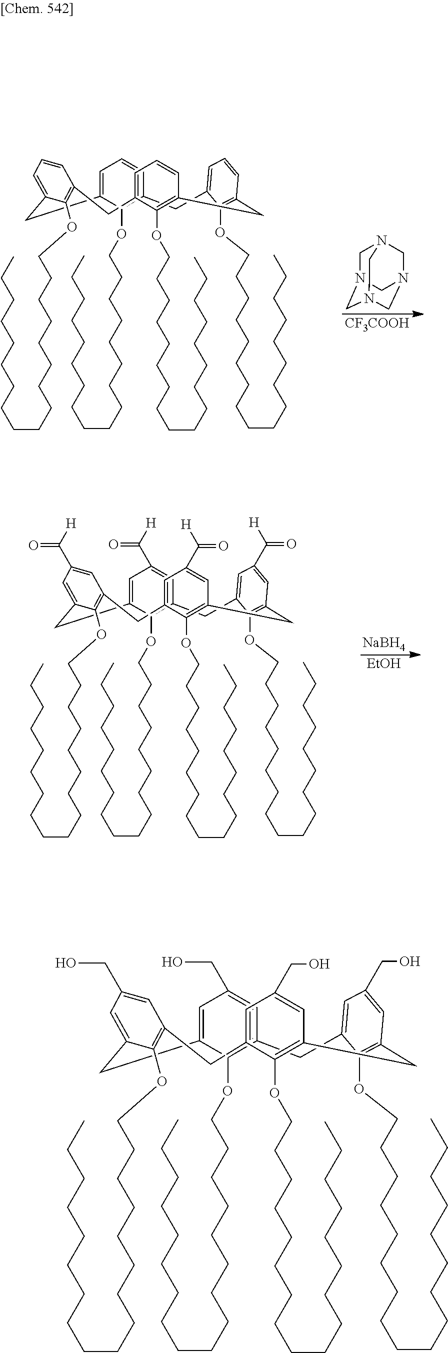

### Synthesis Example 54

A 500 mL four-necked flask equipped with a stirrer, a dropping funnel, a thermometer, and a reflux condenser tube was charged, in a nitrogen atmosphere, with sodium hydride (3.28 g, 82.1 mmol), and mineral oil was removed by washing with hexane. Then dry DMF (100 mL) and hexyl bromide (16.2 g, 90.3 mmol) were added, and the resulting mixture was heated to 70° C. under stirring. Then a solution prepared by dissolving in dry DMF (40 mL) 5,11,17,23-tetraallyl-25,26,27,28-tetrahydroxycalix[4]arene (6.0 g, 10.3 mmol) synthesized by a method described in a known document (The Journal of Organic Chemistry 50, 5802-58061; 1985) was added from a dropping funnel. After completion of the addition, the mixture was continuously stirred for additional 2 hours. The reaction mixture was cooled to room temperature and then poured onto ice (200 g). Concentrated hydrochloric acid was added to acidify the aqueous solution, and the resulting solution was extracted with chloroform (150 mL) twice. The chloroform solution was washed with water until the pH was 5 or higher, further washed with saturated brine, and dried over anhydrous magnesium sulfate. The solvent was removed using an evaporator to thereby obtain a yellow liquid. The yellow liquid was purified by silica gel column chromatography to thereby obtain a colorless transparent solution, and a compound represented by the following formula was obtained as a white solid by recrystallization (6.6 g, yield 70%).

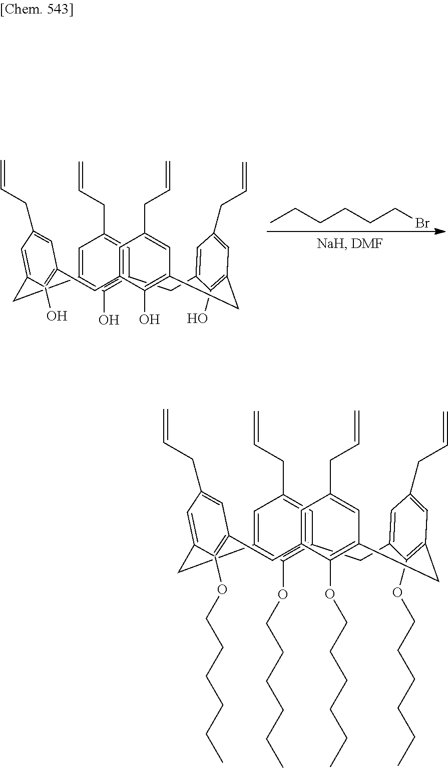

### Synthesis Example 55

The same procedure as in Synthesis Example 54 was repeated except that methyl iodide was used instead of hexyl bromide and that the reaction was performed at room temperature for 24 hours, to thereby obtain a compound represented by the following formula (4.27 g, yield 65%).

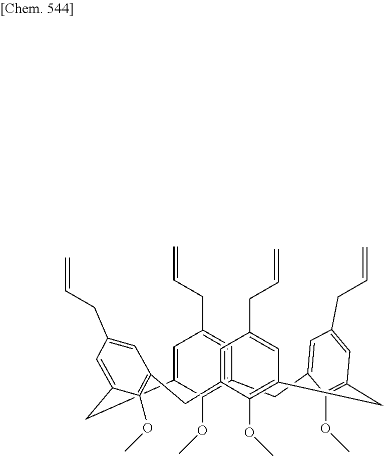 [SMILES: C=CCc1cc2c(OC)c(c1)Cc1cc(CC=C)cc(c1OC)Cc1cc(CC=C)cc(c1OC)Cc1cc(CC=C)cc(c1OC)C2]

### Synthesis Example 56

The same procedure as in Synthesis Example 54 was repeated except that butyl bromide was used instead of hexyl bromide, to thereby obtain a compound represented by the following formula (6.23 g, yield 75%).

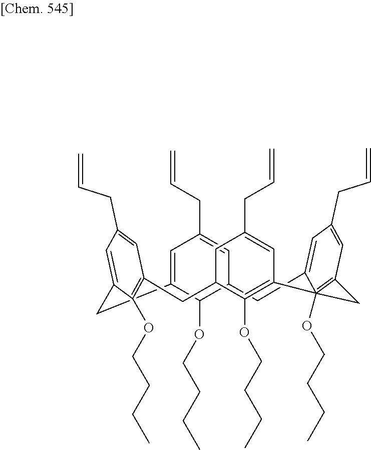

The same procedure as in Synthesis Example 54 was repeated except that heptyl bromide was used instead of hexyl bromide, to thereby obtain a compound represented by the following formula (8.02 g, yield 80%).

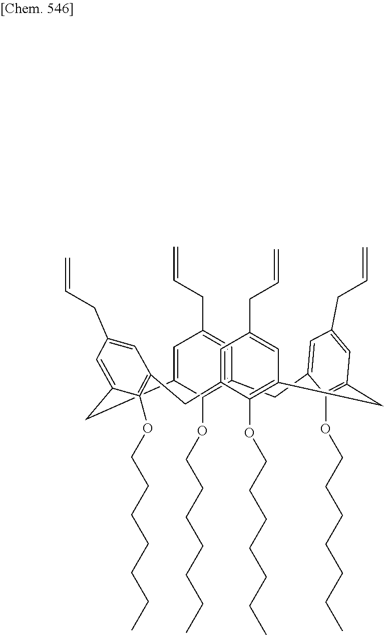 [SMILES: C=CCc1cc2c(OCCCCCCC)c(c1)Cc1cc(CC=C)cc(c1OCCCCCCC)Cc1cc(CC=C)cc(c1OCCCCCCC)Cc1cc(CC=C)cc(c1OCCCCCCC)C2]

### Synthesis Example 58

The same procedure as in Synthesis Example 54 was repeated except that octadecyl bromide was used instead of hexyl bromide, to thereby obtain a compound represented by the following formula (12.8 g, yield 75%).

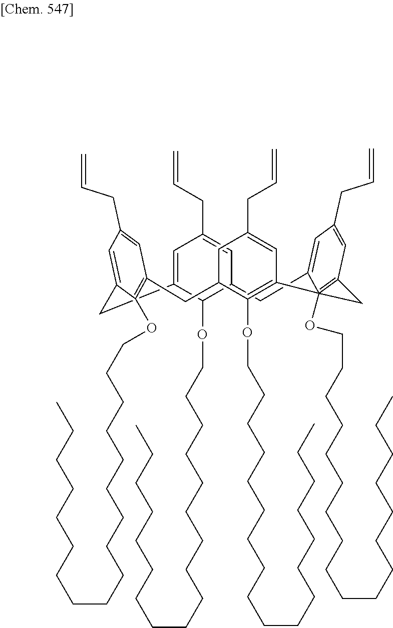

### Synthesis Example 59

Referring to a known document (The Journal of Organic Chemistry, 67, 4722-4733; 2002), a compound represented by the following formula was synthesized using the compound obtained in Synthesis Example 54 (4 g, 4.34 mmol) (yield amount 2.93 g, yield 68%).

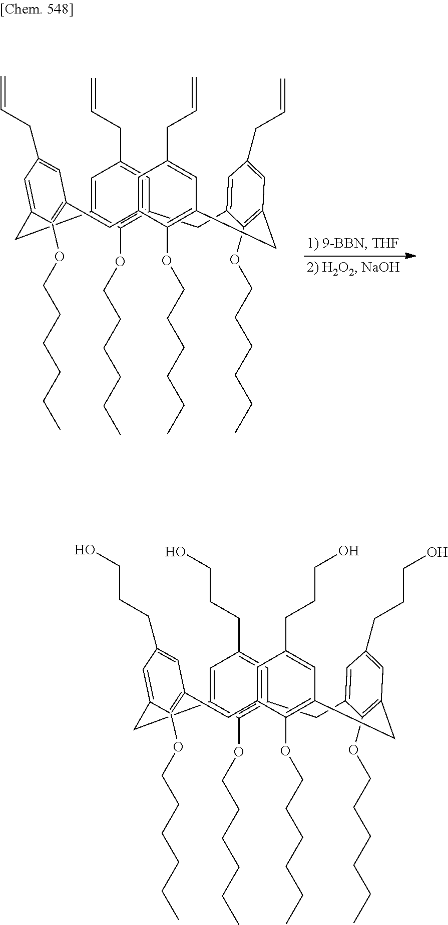

### Synthesis Example 60

The same procedure as in Synthesis Example 59 was repeated except that the compound obtained in Synthesis Example 55 (4.0 g, 6.24 mmol) was used instead of the compound obtained in Synthesis Example 54, to thereby obtain a compound represented by the following formula (4.5 g, yield 72%).

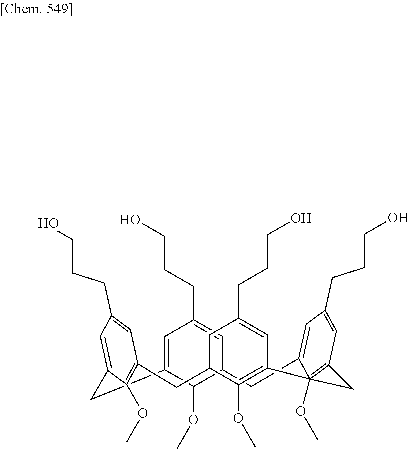

### Synthesis Example 61

The same procedure as in Synthesis Example 59 was repeated except that the compound obtained in Synthesis Example 56 (4.0 g, 4.94 mmol) was used instead of the compound obtained in Synthesis Example 54, to thereby obtain a compound represented by the following formula (2.59 g, yield 65%).

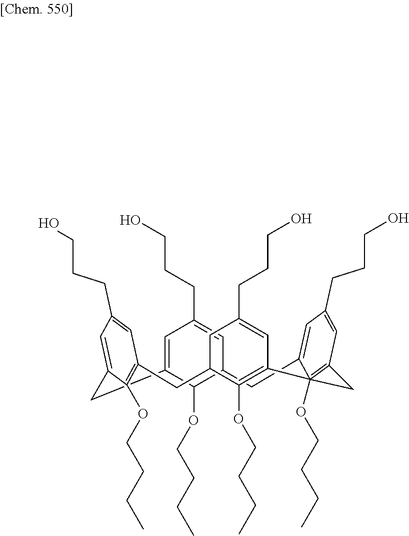

### Synthesis Example 62

The same procedure as in Synthesis Example 59 was repeated except that the compound obtained in Synthesis Example 57 (4.0 g, 4.11 mmol) was used instead of the compound obtained in Synthesis Example 54, to thereby obtain a compound represented by the following formula (3.23 g, yield 75%).

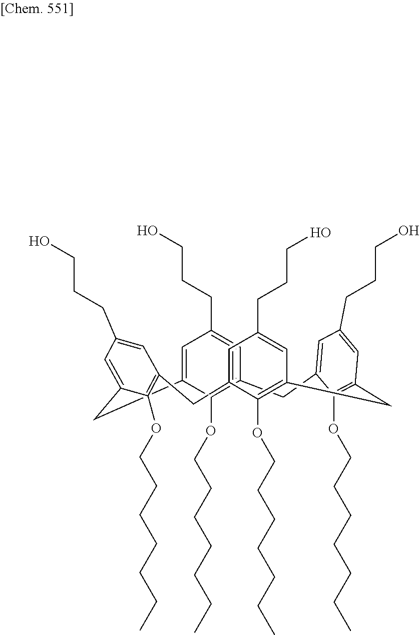 [SMILES: CCCCCCCOc1c2cc(CCCO)cc1Cc1cc(CCCO)cc(c1OCCCCCCC)Cc1cc(CCCO)cc(c1OCCCCCCC)Cc1cc(CCCO)cc(c1OCCCCCCC)C2]

### Synthesis Example 63

The same procedure as in Synthesis Example 59 was repeated except that the compound obtained in Synthesis Example 57 (8.0 g, 5.02 mmol) was used instead of the compound obtained in Synthesis Example 54, to thereby obtain a compound represented by the following formula (5.1 g, yield 61%)

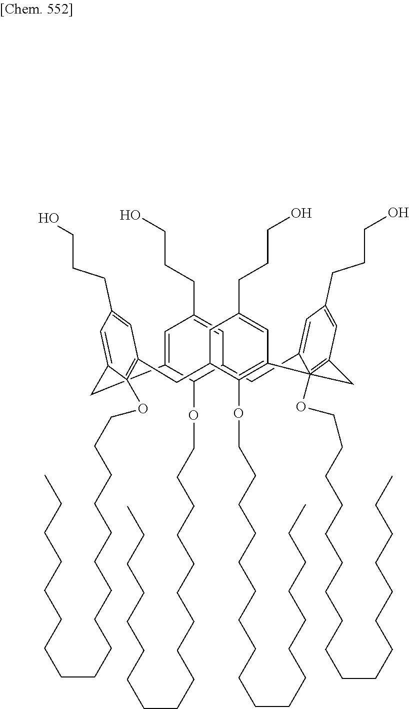

### Example 31

A 100 mL four-necked flask equipped with a stirrer, a dropping funnel, and a thermometer was charged, in a nitrogen atmosphere, with the compound obtained in Synthesis Example 49 (3.0 g, 3.94 mmol), triethylamine (3.19 g, 31.52 mmol), and methylene chloride (35.5 mL), and the mixture was stirred under cooling with ice. A solution prepared by dissolving acrylic acid chloride (0.856 g, 9.46 mmol) and methyl oxal chloride (1.158 g, 9.46 mmol) in methylene chloride (5 mL) was slowly added dropwise. After completion of the dropwise addition, the mixture was stirred at room temperature for 8 hours. Water was added to the reaction mixture, and the resulting mixture was extracted with chloroform (50 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator to thereby obtain a yellow liquid. The yellow liquid was purified by silica gel column chromatography to thereby obtain the target compounds 01-6, 02-6, 03-6, and 04-6 as follows. 01-6 (0.681 g, yield 15.8%). A mixture of 02-6 and 03-6 (2.554 g, yield 55.8%). 04-6 (0.601 g, yield 13.5%).

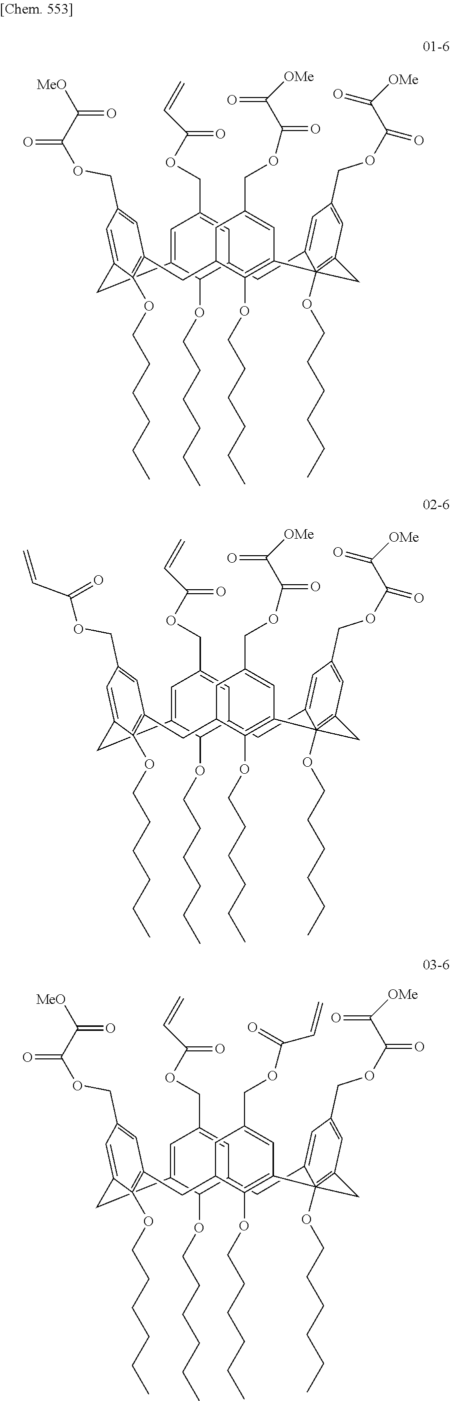

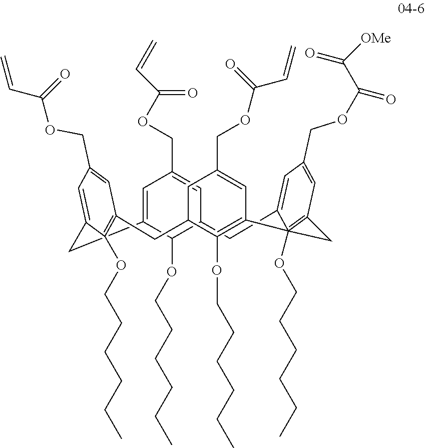

### Example 32

The same procedure as in Example 31 was repeated except that the compound obtained in Synthesis Example 50 (3.0 g, 4.99 mmol) was used instead of the compound obtained in Synthesis Example 4, to thereby obtain the target compounds 01-1, 02-1, 03-1, and 04-1 as follows. 01-1 (0.666 g, yield 14.6%). A mixture of 02-1 and 03-1 (2.222 g, yield 50.5%). 04-1 (0.649 g, yield 15.3%).

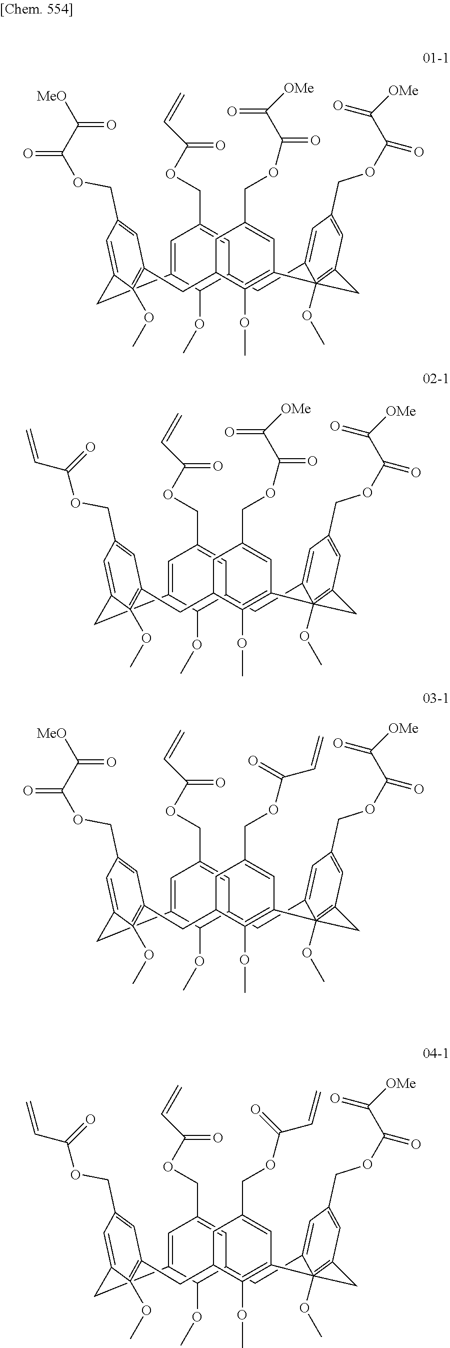

### Example 33

The same procedure as in Example 31 was repeated except that the compound obtained in Synthesis Example 51 (3.0 g, 3.9 mmol) was used instead of the compound obtained in Synthesis Example 49, to thereby obtain the target compounds 01-4, 02-4, 03-4, and 04-4 as follows. 01-4 (0.557 g, yield 13.2%). A mixture of 02-4 and 03-4 (2.190 g, yield 53.5%). 04-4 (0.627 g, yield 15.8%).

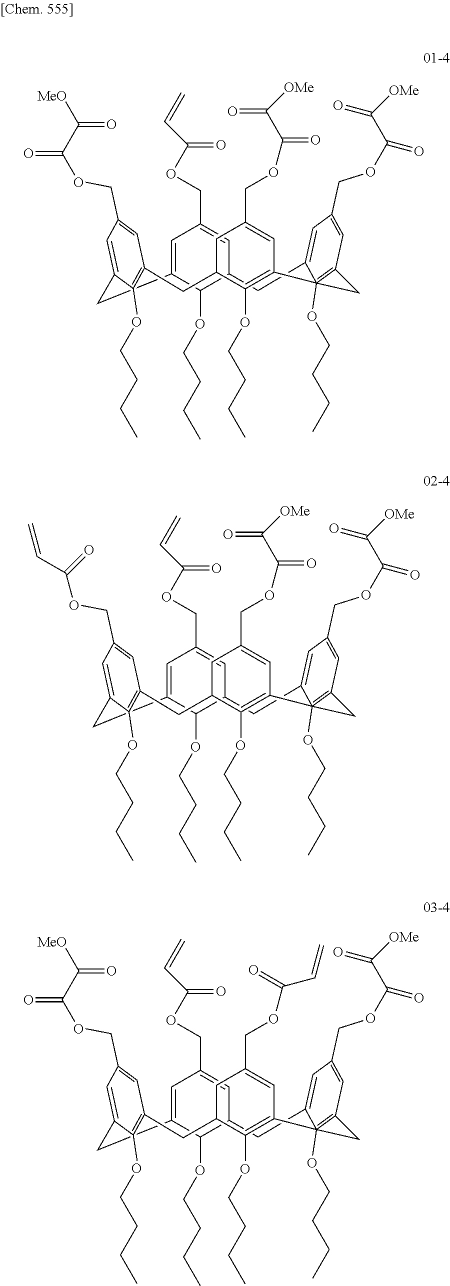

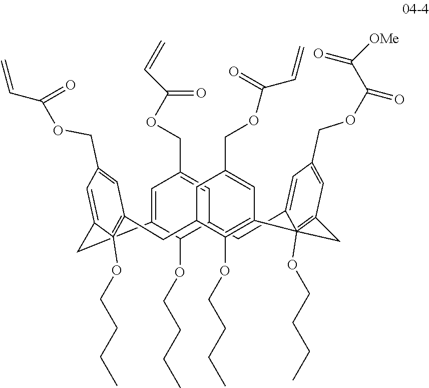

### Example 34

The same procedure as in Example 31 was repeated except that the compound obtained in Synthesis Example 52 (3.0 g, 3.2 mmol) was used instead of the compound obtained in Synthesis Example 49, to thereby obtain the target compounds 01-7, 02-7, 03-7, and 04-7 as follows. 01-7 (0.580 g, yield 14.5%). A mixture of 02-7 and 03-7 (2.174 g, yield 55.8%). 04-7 (0.429 g, yield 11.3%).

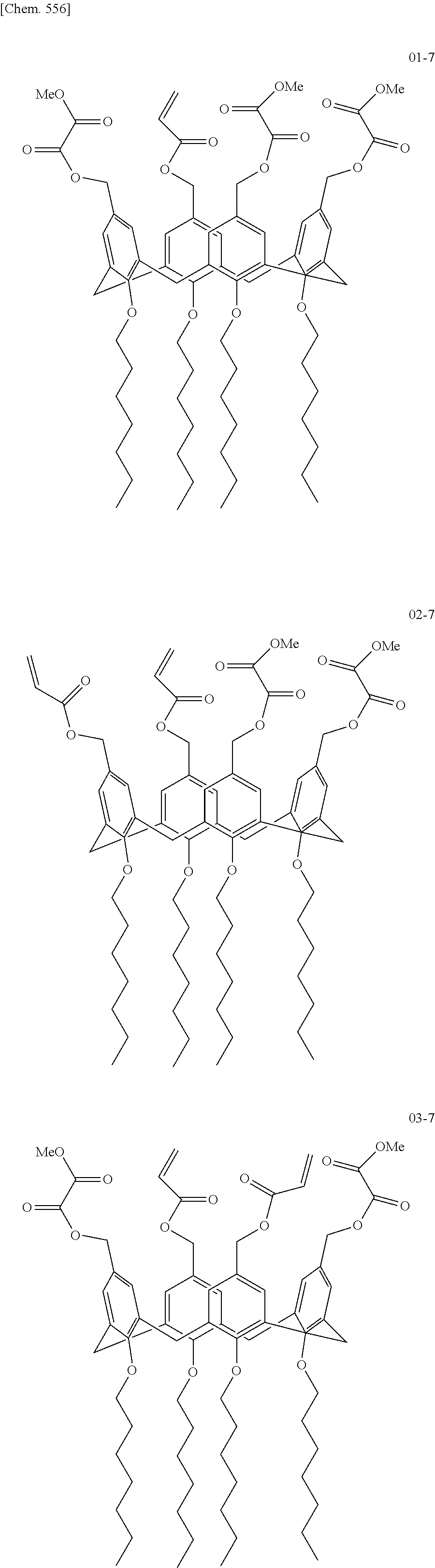

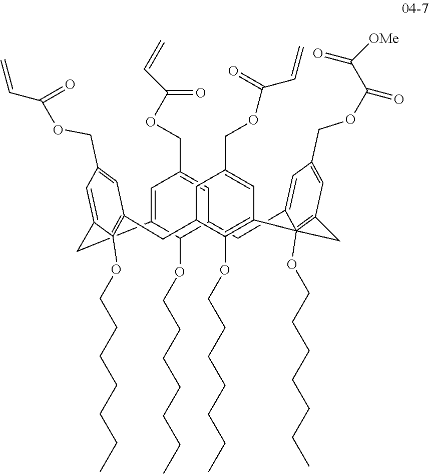

### Example 35

The same procedure as in Example 31 was repeated except that the compound obtained in Synthesis Example 53 (3.0 g, 1.93 mmol) was used instead of the compound obtained in Synthesis Example 49, to thereby obtain the target compounds 01-18, 02-18, 03-18, and 04-18 as follows. 01-18 (0.371 g, yield 10.3%). A mixture of 02-18 and 03-18 (1.816 g, yield 51.3%). 04-18 (0.644 g, yield 18.5%).

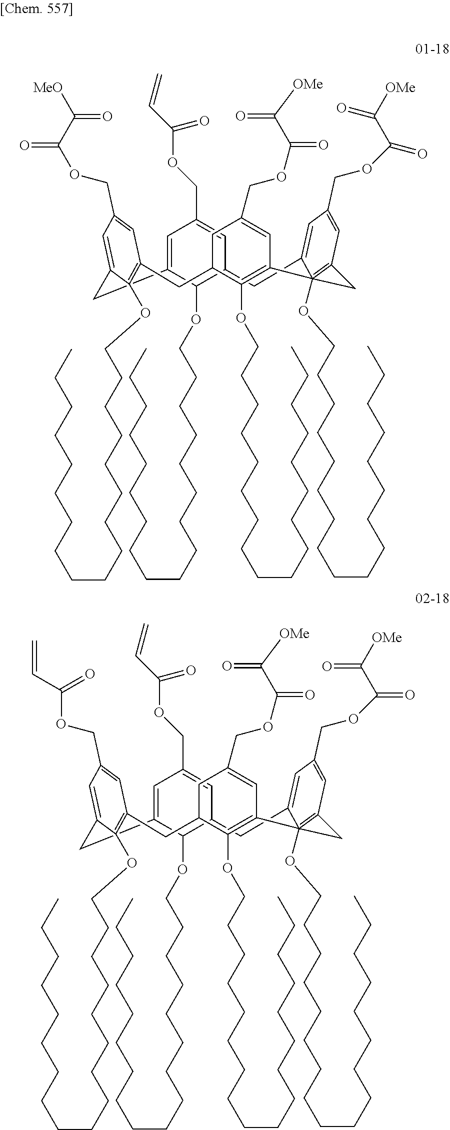

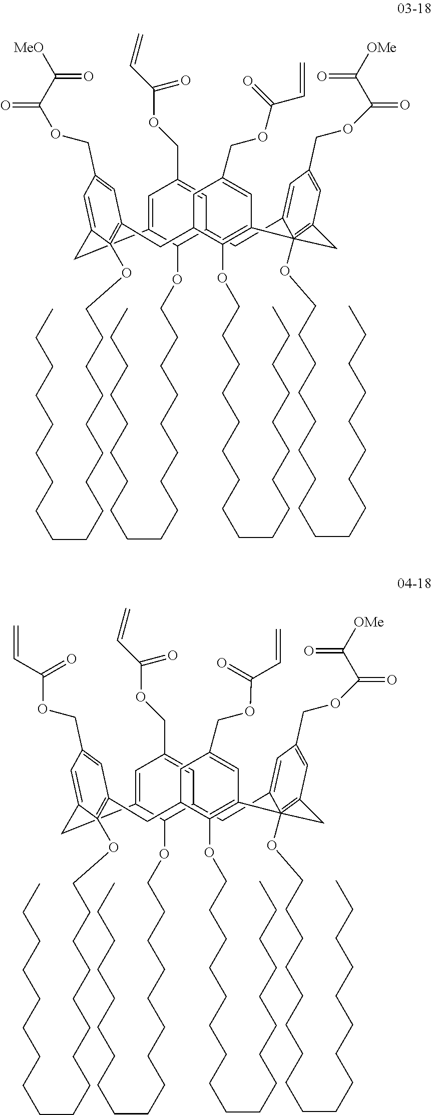

### Synthesis Example 64

A 100 mL four-necked flask equipped with a stirrer, a thermometer, and a reflux condenser tube was charged with 2.00 g (2.27 mmol) of the compound obtained in Synthesis Example 49, 3.57 g (13.62 mmol) of triphenylphosphine, 2.95 g (13.62 mmol) of 2-[[(1,1-dimethylethyl)dimethylsilyl]oxy]2-propenoic acid, and 38 mL of tetrahydrofuran, and the mixture was stirred. Then 2.75 g (13.62 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 2.85 g, yield 75.0%).

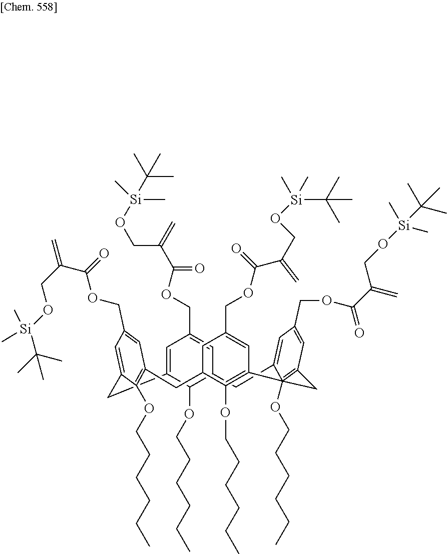

### Synthesis Example 65

The same procedure as in Synthesis Example 64 was repeated except that the compound obtained in Synthesis Example 50 (2.00 g, 3.33 mmol) was used instead of the compound obtained in Synthesis Example 49, to thereby obtain a compound represented by the following formula (3.26 g, yield 70.2%).

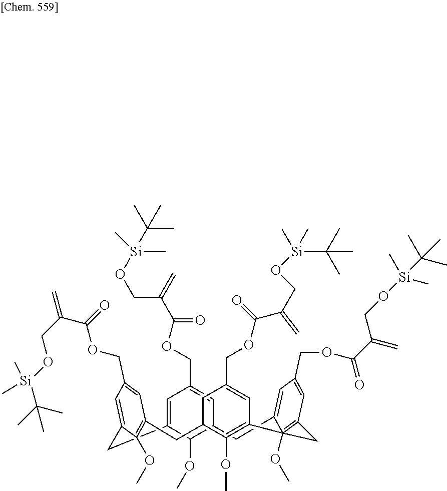

### Synthesis Example 66

The same procedure as in Synthesis Example 64 was repeated except that the compound obtained in Synthesis Example 51 (2.00 g, 2.60 mmol) was used instead of the compound obtained in Synthesis Example 49, to thereby obtain a compound represented by the following formula (3.12 g, yield 76.8%).

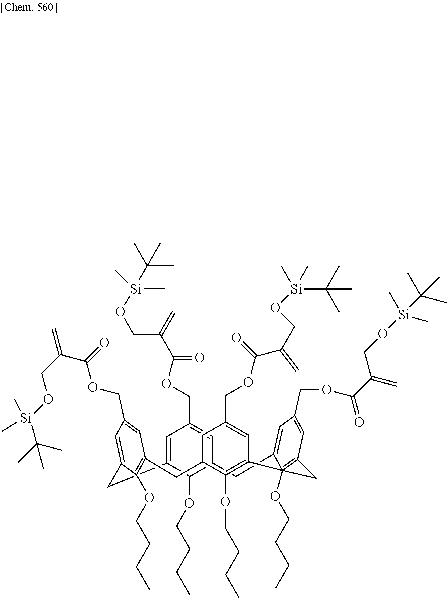

### Synthesis Example 67

The same procedure as in Synthesis Example 64 was repeated except that the compound obtained in Synthesis Example 52 (2.00 g, 2.13 mmol) was used instead of the compound obtained in Synthesis Example 49, to thereby obtain a compound represented by the following formula (2.74 g, yield 74.2%).

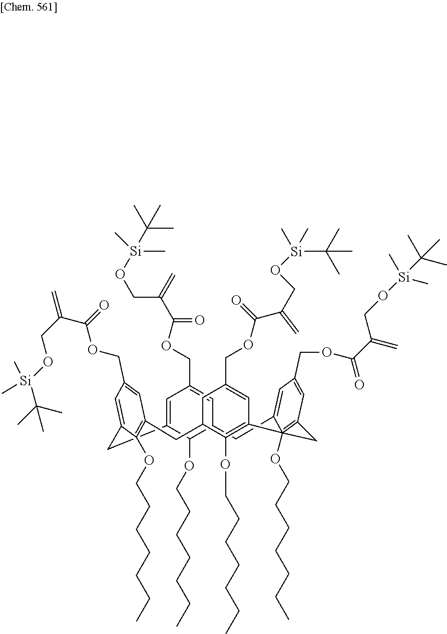

### Synthesis Example 68

The same procedure as in Synthesis Example 62 was repeated except that the compound obtained in Synthesis Example 53 (2.00 g, 1.29 mmol) was used instead of the compound obtained in Synthesis Example 49, to thereby obtain a compound represented by the following formula (2.58 g, yield 85.3%).

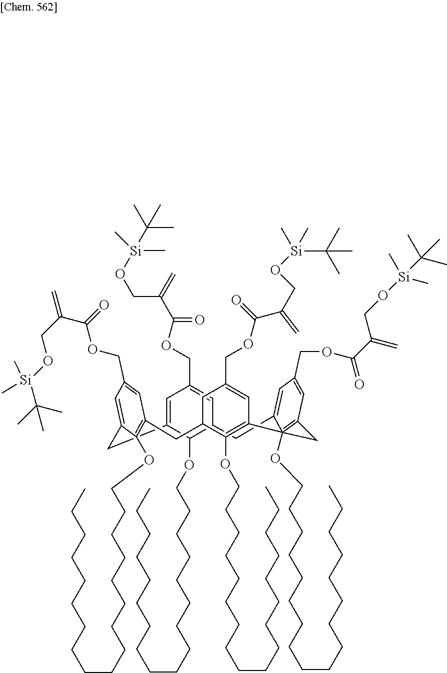

### Synthesis Example 69

A 100 mL four-necked flask equipped with a stirrer, a thermometer, and a reflux condenser tube was charged with 2.50 g (1.49 mmol) of the compound obtained in Synthesis Example 64, 0.538 g (8.96 mmol) of acetic acid, and 60 mL of tetrahydrofuran, and the mixture was stirred. A colorless transparent solution. Then tetrabutylammonium fluoride (an about 1 mol/L tetrahydrofuran solution 8.96 mL (8.96 mmol) was slowly added dropwise to the mixture placed in an ice bath under stirring, and the resulting mixture was further stirred at room temperature for 12 hours. A saturated aqueous ammonium chloride solution was added to the reaction mixture, and then 30 mL of chloroform was added. The reaction mixture was transferred to a separatory funnel, and the organic layer was separated. Then the aqueous layer was extracted with 30 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula as a white solid (yield amount 1.663 g, yield 91.5%).

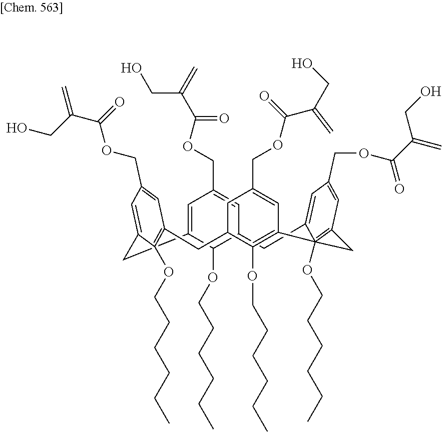

### Synthesis Example 70

The same procedure as in Synthesis Example 69 was repeated except that the compound obtained in Synthesis Example 65 (2.5 g, 1.79 mmol) was used instead of the compound obtained in Synthesis Example 64, to thereby obtain a compound represented by the following formula (1.551 g, yield 92.3%).

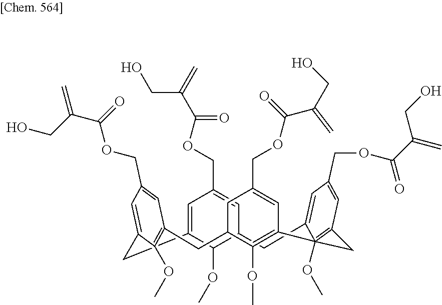

### Synthesis Example 71

The same procedure as in Synthesis Example 69 was repeated except that the compound obtained in Synthesis Example 66 (2.5 g, 1.60 mmol) was used instead of the compound obtained in Synthesis Example 64, to thereby obtain a compound represented by the following formula (1.671 g,

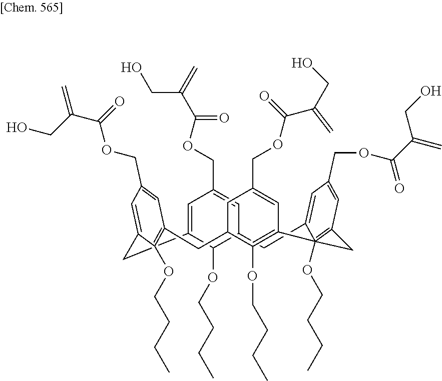

### Synthesis Example 72

The same procedure as in Synthesis Example 69 was repeated except that the compound obtained in Synthesis Example 67 (2.5 g, 1.44 mmol) was used instead of the compound obtained in Synthesis Example 64, to thereby obtain a compound represented by the following formula (1.759 g, yield 95.6%).

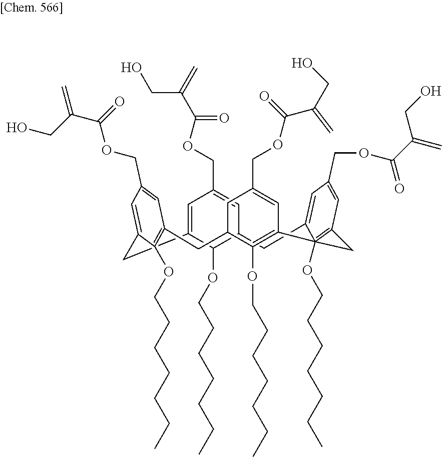

### Synthesis Example 73

The same procedure as in Synthesis Example 69 was repeated except that the compound obtained in Synthesis Example 68 (2.50 g, 1.06 mmol) was used instead of the compound obtained in Synthesis Example 64, to thereby obtain a compound represented by the following formula (1.90 g, yield 94.8%).

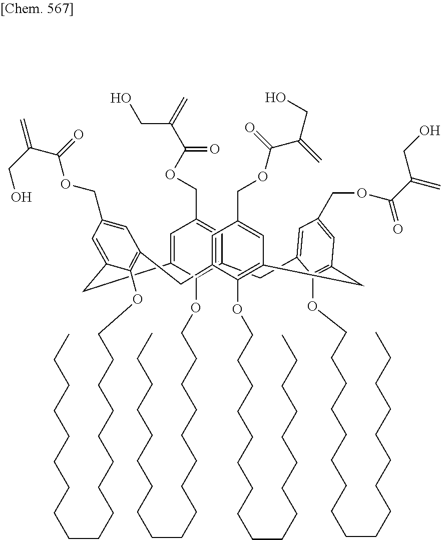 [SMILES: C=C(CO)C(=O)OCc1cc2c(OCCCCCCCCCCCCCCCCCC)c(c1)Cc1cc(COC(=O)C(=C)CO)cc(c1OCCCCCCCCCCCCCCCCCC)Cc1cc(COC(=O)C(=C)CO)cc(c1OCCCCCCCCCCCCCCCCCC)Cc1cc(COC(=O)C(=C)CO)cc(c1OCCCCCCCCCCCCCCCCCC)C2]

### Example 36

A 100 mL four-necked flask equipped with a stirrer, a dropping funnel, and a thermometer was charged, in a nitrogen atmosphere, with the compound obtained in Synthesis Example 69 (1.5 g, 1.23 mmol), triethylamine (0.997 g, 9.86 mmol), and methylene chloride (15 mL), and the mixture was stirred under cooling with ice. Then a solution prepared by dissolving methyl oxal chloride (0.906 g, 7.39 mmol) in methylene chloride (3 mL) was slowly added dropwise. After completion of the dropwise addition, the resulting mixture was stirred at room temperature for 8 hours. Water was added to the reaction mixture, and the resulting mixture was extracted with chloroform (40 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator, and the obtained yellow viscous liquid was purified by silica gel column chromatography to thereby obtain the target compound 05-6 (yield amount 1.664 g, yield 86.5%).

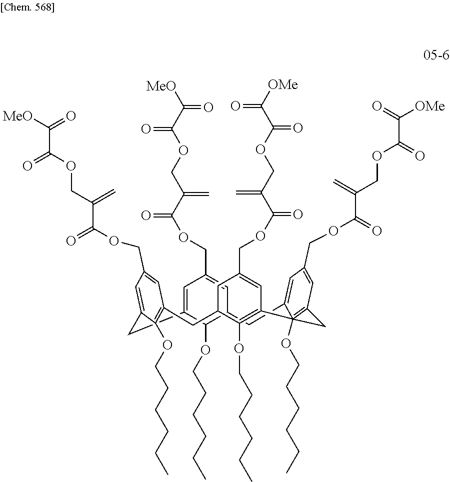

### Example 37

The same procedure as in Example 36 was repeated except that the compound obtained in Synthesis Example 70 (1.50 g, 1.60 mmol) was used instead of the compound obtained in Synthesis Example 69, to thereby obtain the target compound 05-1 (1.688 g, yield 82.3%).

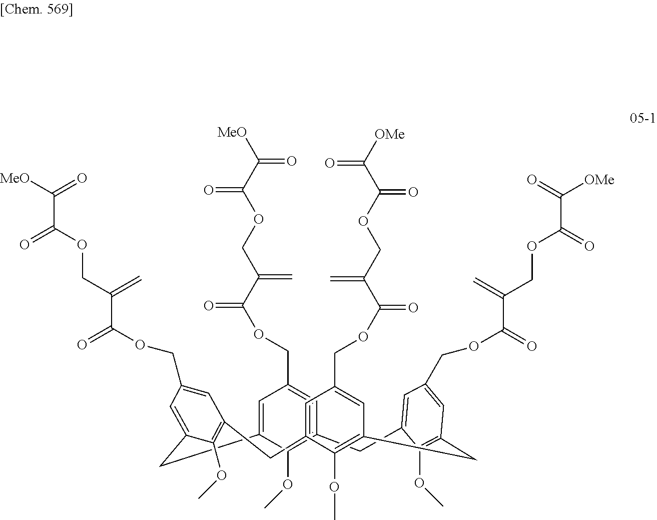 [SMILES: C=C(COC(=O)C(=O)OC)C(=O)OCc1cc2c(OC)c(c1)Cc1cc(COC(=O)C(=C)COC(=O)C(=O)OC)cc(c1OC)Cc1cc(COC(=O)C(=C)COC(=O)C(=O)OC)cc(c1OC)Cc1cc(COC(=O)C(=C)COC(=O)C(=O)OC)cc(c1OC)C2]

### Example 38

The same procedure as in Example 36 was repeated except that the compound obtained in Synthesis Example 71 (1.50 g, 1.36 mmol) was used instead of the compound obtained in Synthesis Example 69, to thereby obtain the target compound 05-4 (1.721 g, yield 87.5%).

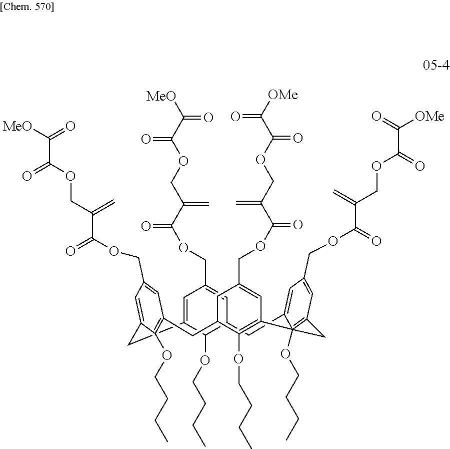

### Example 39

The same procedure as in Example 36 was repeated except that the compound obtained in Synthesis Example 72 (1.50 g, 1.18 mmol) was used instead of the compound obtained in Synthesis Example 69, to thereby obtain the target compound 05-7 (1.734 g, yield 91.0%).

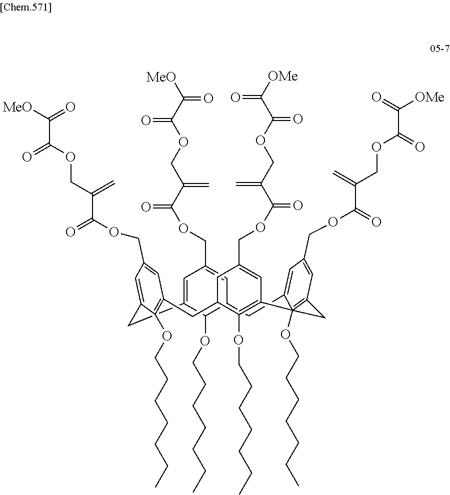

### Example 40

The same procedure as in Example 36 was repeated except that the compound obtained in Synthesis Example 73 (1.5 g, 0.79 mmol) was used instead of the compound obtained in Synthesis Example 69, to thereby obtain the target compound 05-18 (1.516 g, yield 85.5%).

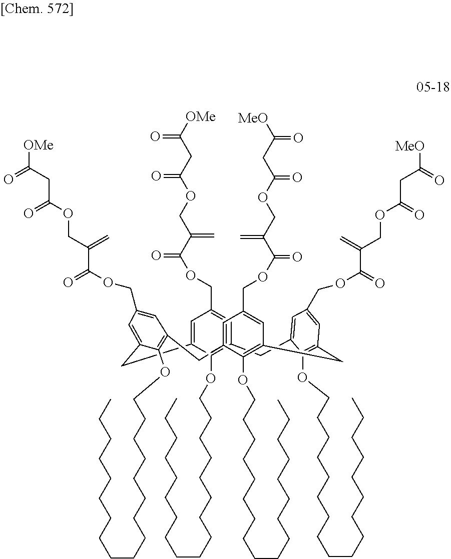 [SMILES: C=C(COC(=O)CC(=O)OC)C(=O)OCc1cc2c(OCCCCCCCCCCCCCCCCCC)c(c1)Cc1cc(COC(=O)C(=C)COC(=O)CC(=O)OC)cc(c1OCCCCCCCCCCCCCCCCCC)Cc1cc(COC(=O)C(=C)COC(=O)CC(=O)OC)cc(c1OCCCCCCCCCCCCCCCCCC)Cc1cc(COC(=O)C(=C)COC(=O)CC(=O)OC)cc(c1OCCCCCCCCCCCCCCCCCC)C2]

### Example 41

A 100 mL four-necked flask equipped with a stirrer, a dropping funnel, and a thermometer was charged, in a nitrogen atmosphere, with the compound obtained in Synthesis Example 59 (3.0 g, 3.02 mmol), triethylamine (2.445 g, 24.16 mmol), and methylene chloride (30.2 mL), and the mixture was stirred under cooling with ice. Then a solution prepared by dissolving acrylic acid chloride (0.656 g, 7.25 mmol) and methyl oxal chloride (0.888 g, 7.25 mmol) in methylene chloride (5 mL) was slowly added dropwise. After completion of the dropwise addition, the resulting mixture was stirred at room temperature for 8 hours. Water was added to the reaction mixture, and the resulting mixture was extracted with chloroform (50 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator to thereby obtain a yellow liquid. The yellow liquid was purified by silica gel column chromatography to thereby obtain the target compounds 06-6, 07-6, 08-6, and 09-6 as follows. 06-6 (0.572 g, yield 14.5%). A mixture of 07-6 and 08-6 (2.054 g, yield 53.4%). 09-6 (0.480 g, yield 12.8%).

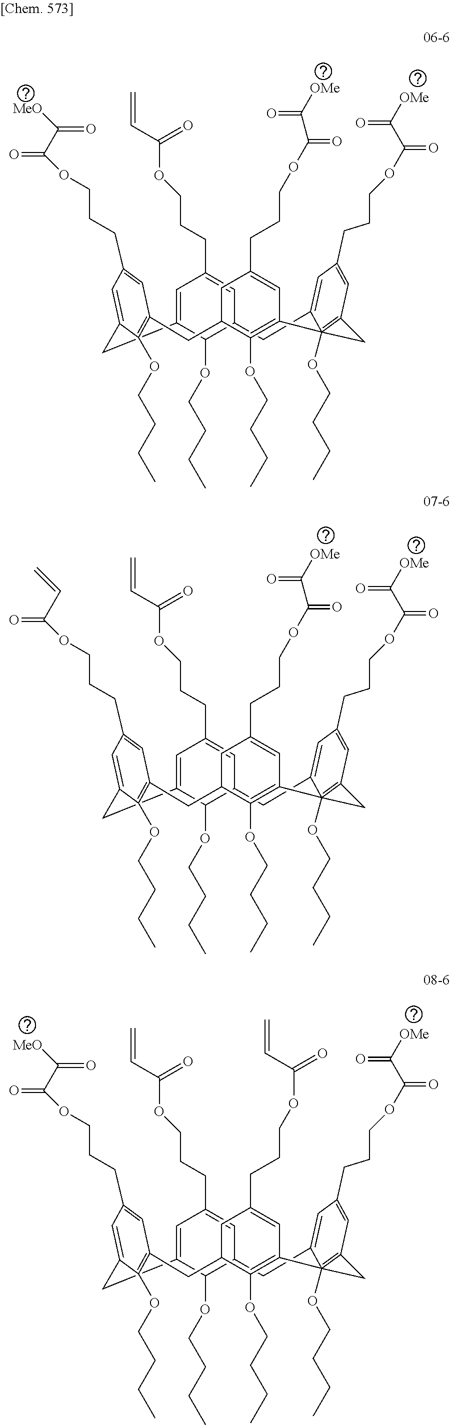

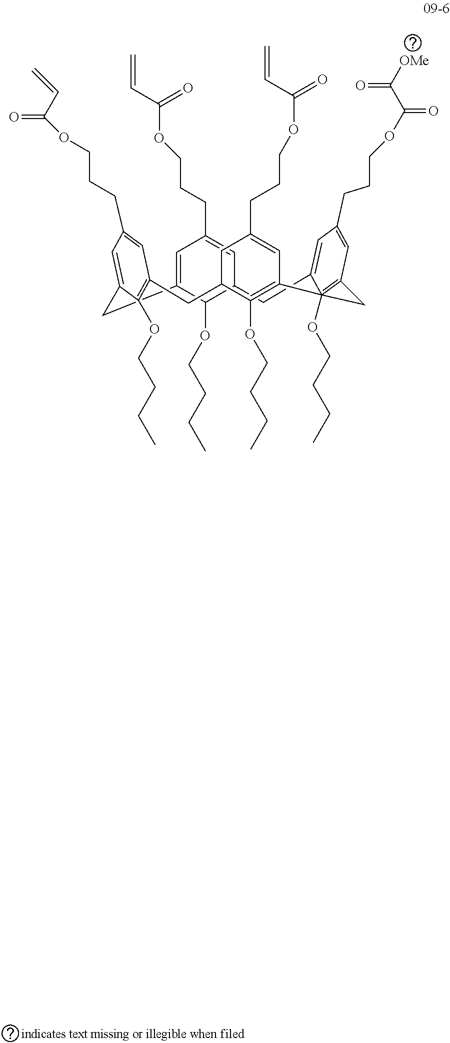

### Example 42

The same procedure as in Example 41 was repeated except that the compound obtained in Synthesis Example 60 (3.00 g, 4.21 mmol) was used instead of the compound obtained in Synthesis Example 59, to thereby obtain the target compounds 06-1, 07-1, 08-1, and 09-1 as follows. 06-1 (0.669 g, yield 15.5%). A mixture of 07-1 and 08-1 (2.152 g, yield 51.5%). 09-1 (0.599 g, yield 14.8%).

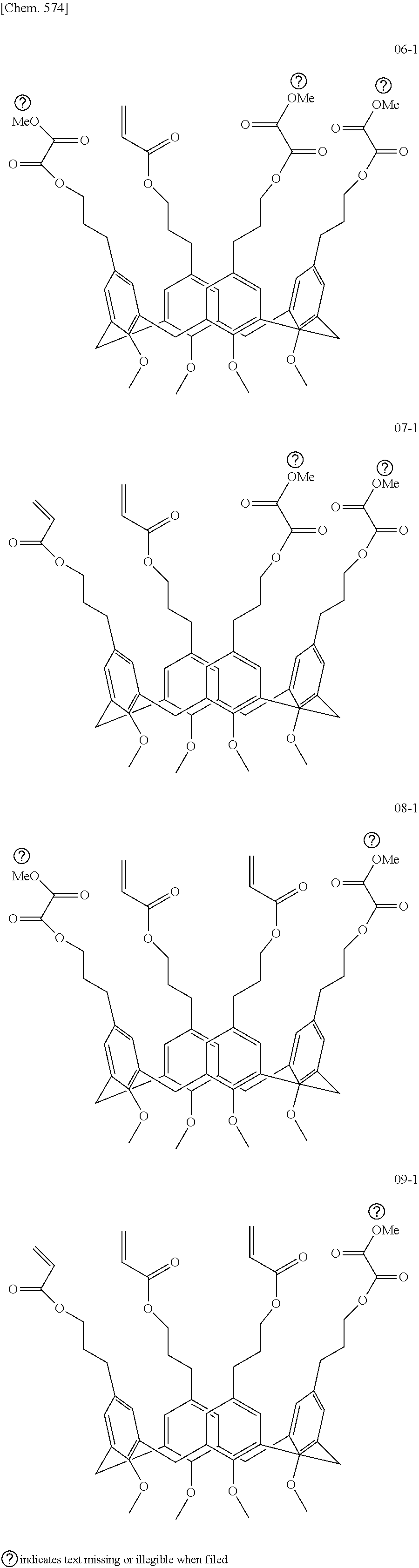

### Example 43

The same procedure as in Example 41 was repeated except that the compound obtained in Synthesis Example 61 (3.00 g, 3.40 mmol) was used instead of the compound obtained in Synthesis Example 59, to thereby obtain the target compounds 06-4, 07-4, 08-4, and 09-4 as follows. 06-4 (0.553 g, yield 13.6%). A mixture of 07-1 and 08-1 (2.139 g, yield 54.1%). 09-4 (0.546 g, yield 14.2%).

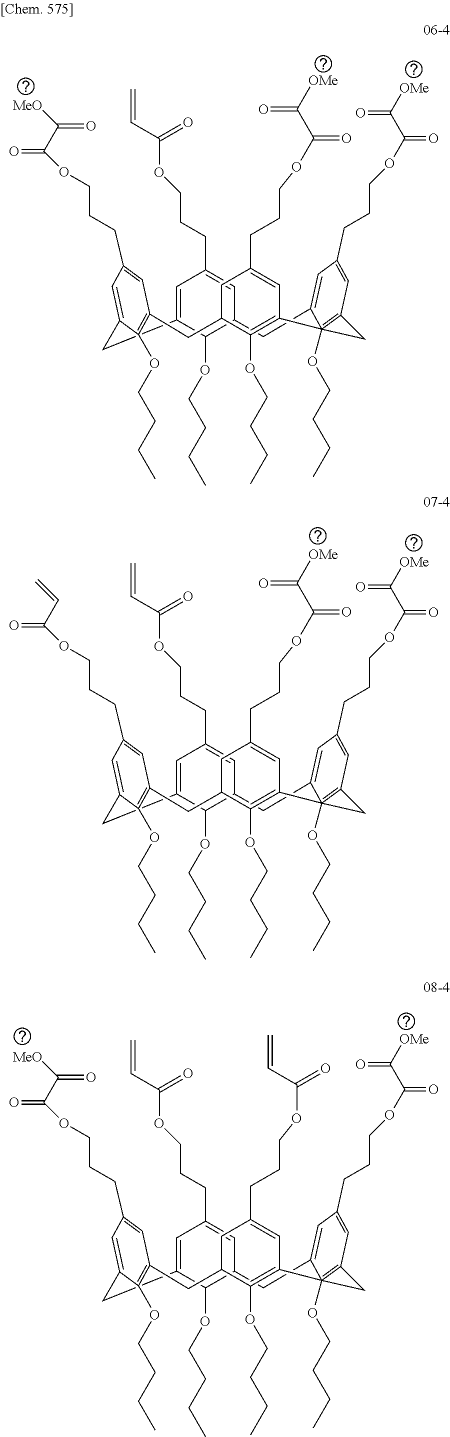

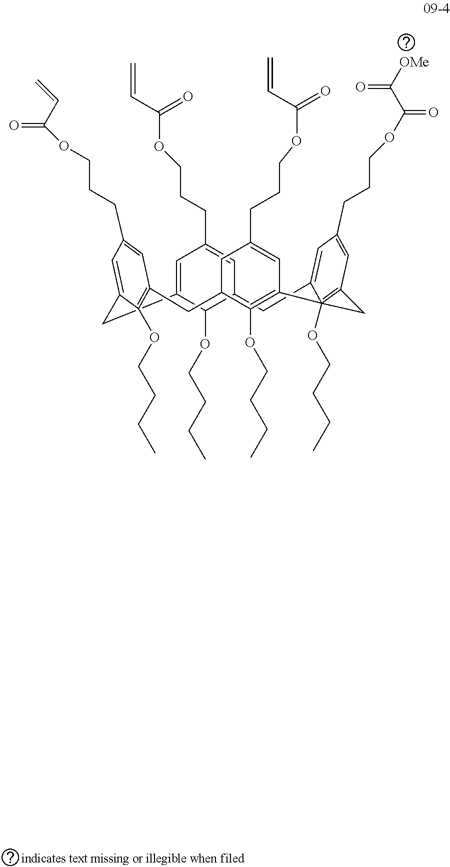

### Example 44

The same procedure as in Example 41 was repeated except that the compound obtained in Synthesis Example 62 (3.00 g, 2.86 mmol) was used instead of the compound obtained in Synthesis Example 59, to thereby obtain the target compounds 06-7, 07-7, 08-7, and 09-7 as follows. 06-7 (0.537 g, yield 13.8%). A mixture of 07-7 and 08-7 (2.083 g, yield 54.8%). 09-7 (0.464 g, yield 12.5%).

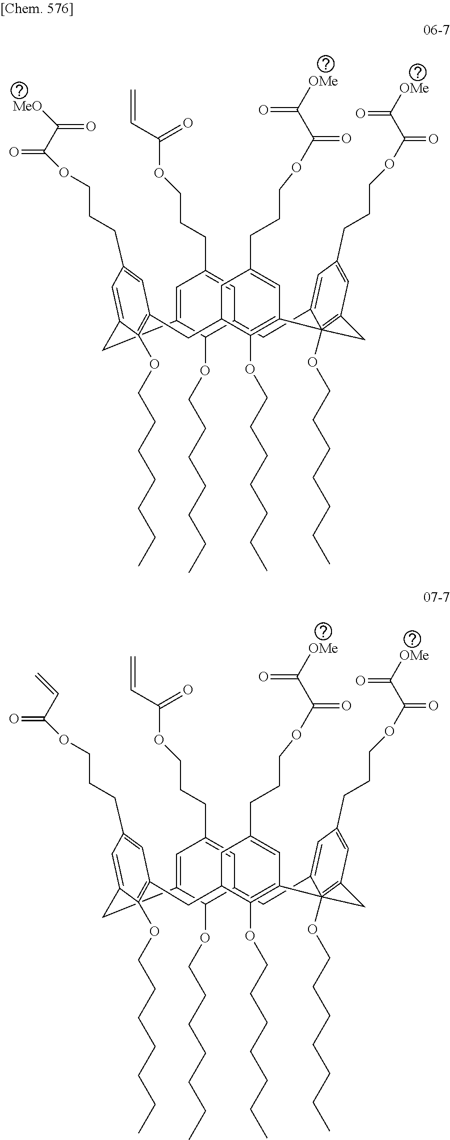

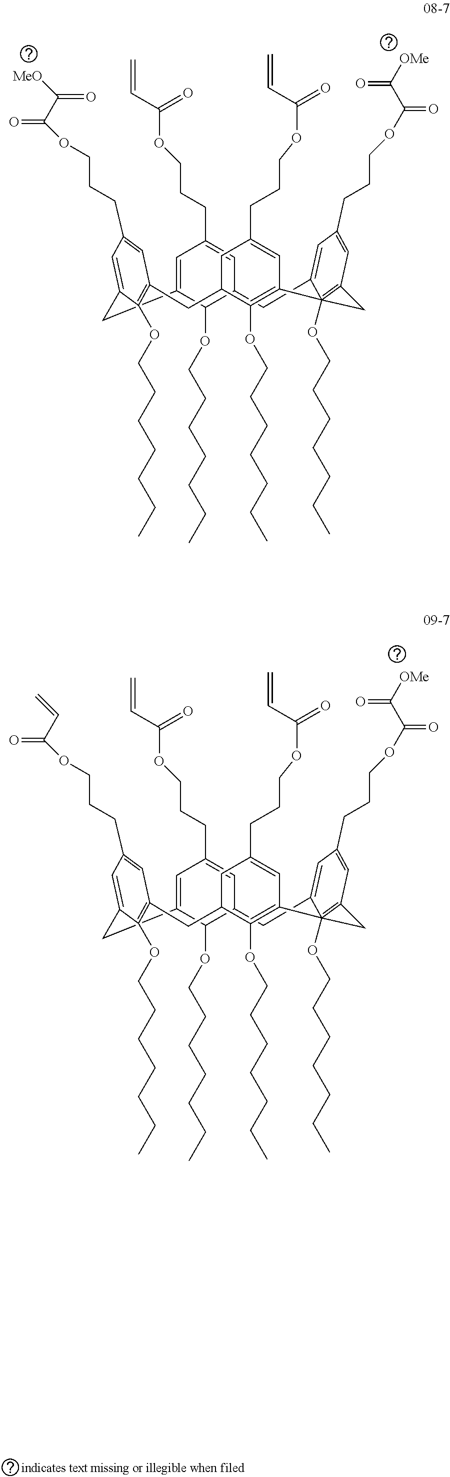

### Example 45

The same procedure as in Example 41 was repeated except that the compound obtained in Synthesis Example 63 (3.00 g, 1.80 mmol) was used instead of the compound obtained in Synthesis Example 59, to thereby obtain the target compounds 06-18, 07-18, 08-18, and 09-18 as follows. 06-18 (0.350 g, yield 10.1%). A mixture of 07-18 and 08-18 (1.719 g, yield 50.5%). 09-18 (0.639 g, yield 19.1%).

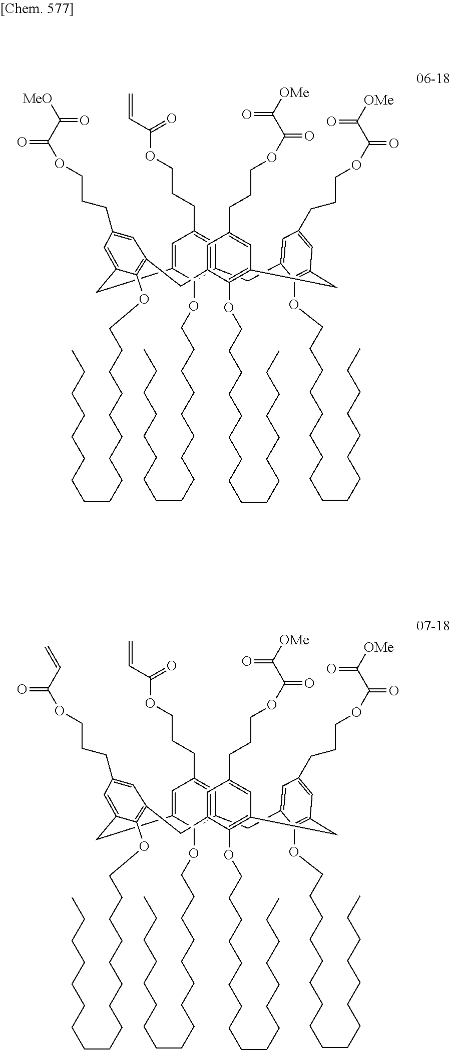 [SMILES: C=CC(=O)OCCCc1cc2c(OCCCCCCCCCCCCCCCCC)c(c1)Cc1cc(CCCOC(=O)C(=O)OC)cc(c1OCCCCCCCCCCCCCCCCC)Cc1cc(CCCOC(=O)C(=O)OC)cc(c1OCCCCCCCCCCCCCCCCC)Cc1cc(CCCOC(=O)C(=O)OC)cc(c1OCCCCCCCCCCCCCCCCC)C2 ; C=CC(=O)OCCCc1cc2c(OCCCCCCCCCCCCCCCCC)c(c1)Cc1cc(CCCOC(=O)C(=O)OC)cc(c1OCCCCCCCCCCCCCCCCC)Cc1cc(CCCOC(=O)C(=O)OC)cc(c1OCCCCCCCCCCCCCCCCC)Cc1cc(CCCOC(=O)C=C)cc(c1OCCCCCCCCCCCCCCCCC)C2]

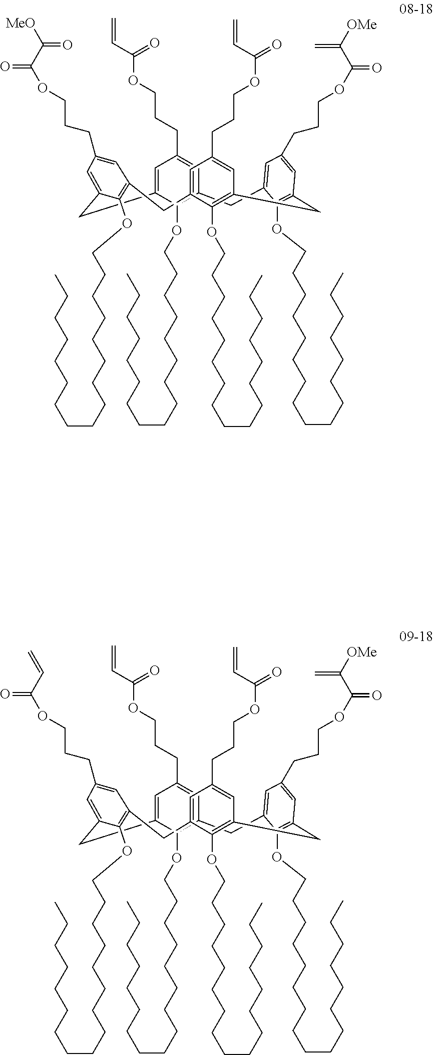 [SMILES: C=CC(=O)OCCCc1cc2c(OCCCCCCCCCCCCCCCCC)c(c1)Cc1cc(CCCOC(=O)C(=O)OC)cc(c1OCCCCCCCCCCCCCCCCC)Cc1cc(CCCOC(=O)C=C)cc(c1OCCCCCCCCCCCCCCCCC)Cc1cc(CCCOC(=O)C(=C)OC)cc(c1OCCCCCCCCCCCCCCCCC)C2 ; C=CC(=O)OCCCc1cc2c(OCCCCCCCCCCCCCCCCC)c(c1)Cc1cc(CCCOC(=O)C=C)cc(c1OCCCCCCCCCCCCCCCCC)Cc1cc(CCCOC(=O)C(=C)OC)cc(c1OCCCCCCCCCCCCCCCCC)Cc1cc(CCCOC(=O)C=C)cc(c1OCCCCCCCCCCCCCCCCC)C2]

### Synthesis Example 74

A 100 mL four-necked flask equipped with a stirrer, a thermometer, and a reflux condenser tube was charged with 2.50 g (2.52 mmol) of the compound obtained in Synthesis Example 59, 3.96 g (15.10 mmol) of triphenylphosphine, 3.267 g (15.10 mmol) of 2-[[[(1,1-dimethylethyl)dimethylsilyl]oxy]2-propenoic acid, and 43 mL of tetrahydrofuran, and the mixture was stirred. Then 3.053 g (15.10 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 3.251 g, yield 72.3%).

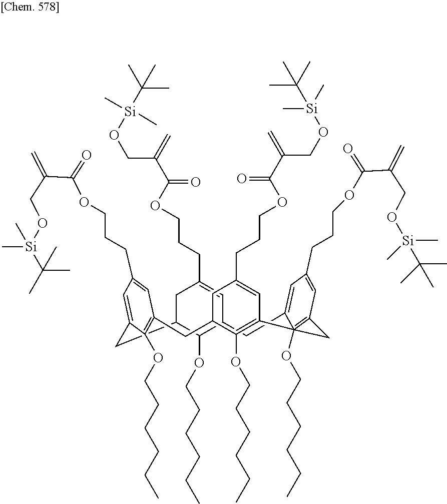

### Synthesis Example 75

The same procedure as in Synthesis Example 74 was repeated except that the compound obtained in Synthesis Example 60 (2.50 g, 3.33 mmol) was used instead of the compound obtained in Synthesis Example 59, to thereby obtain a compound represented by the following formula (3.782 g, yield 71.6%).

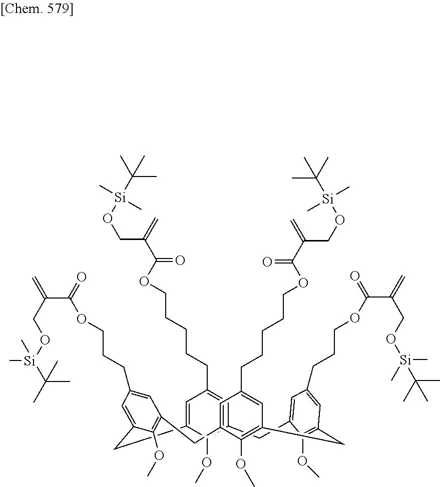 [SMILES: C=C(CO[Si](C)(C)C(C)(C)C)C(=O)OCCCCCc1cc2c(OC)c(c1)Cc1cc(CCCOC(=O)C(=C)CO[Si](C)(C)C(C)(C)C)cc(c1OC)Cc1cc(CCCCCOC(=O)C(=C)CO[Si](C)(C)C(C)(C)C)cc(c1OC)Cc1cc(CCCOC(=O)C(=C)CO[Si](C)(C)C(C)(C)C)cc(c1OC)C2]

### Synthesis Example 76

The same procedure as in Synthesis Example 74 was repeated except that the compound obtained in Synthesis Example 61 (2.50 g, 2.84 mmol) was used instead of the compound obtained in Synthesis Example 59, to thereby obtain a compound represented by the following formula (3.553 g, yield 74.8%).

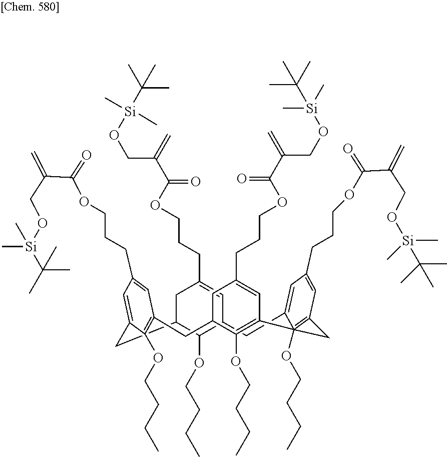

### Synthesis Example 77

The same procedure as in Synthesis Example 74 was repeated except that the compound obtained in Synthesis Example 62 (2.50 g, 2.38 mmol) was used instead of the compound obtained in Synthesis Example 59, to thereby obtain a compound represented by the following formula (3.305 g, yield 75.3%).

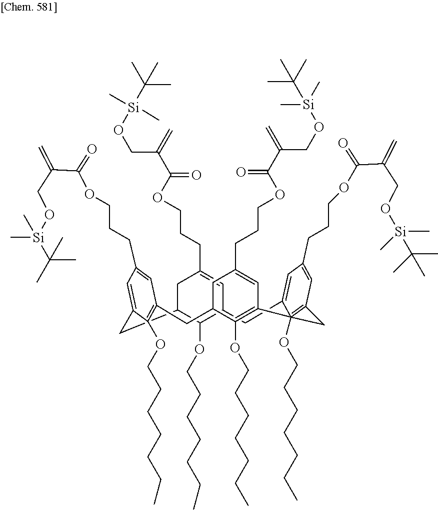

### Synthesis Example 78

The same procedure as in Synthesis Example 74 was repeated except that the compound obtained in Synthesis Example 63 (2.50 g, 1.50 mmol) was used instead of the compound obtained in Synthesis Example 59, to thereby obtain a compound represented by the following formula (3.011 g, yield 81.6%).

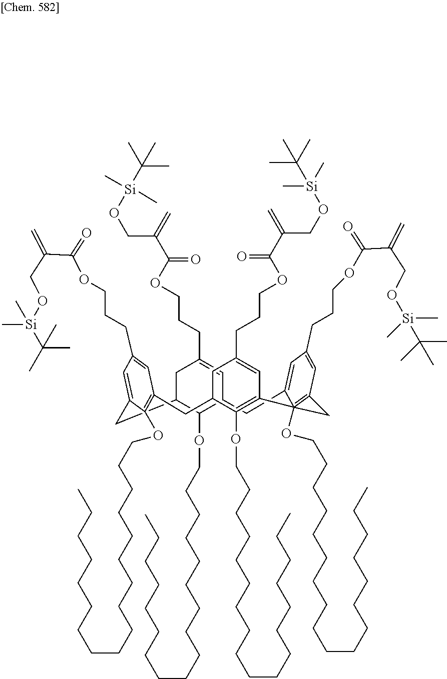

### Synthesis Example 79

A 200 mL four-necked flask equipped with a stirrer, a thermometer, and a reflux condenser tube was charged with 3.50 g (1.96 mmol) of the compound obtained in Synthesis Example 74, 0.706 g (11.75 mmol) of acetic acid, and 78.4 mL of tetrahydrofuran, and the mixture was stirred. A colorless transparent solution. Then tetrabutylammonium fluoride (an about 1 mol/L tetrahydrofuran solution 11.75 mL (11.75 mmol) was slowly added dropwise to the mixture placed in an ice bath under stirring. The resulting mixture was stirred at room temperature for 12 hours. A saturated aqueous ammonium chloride solution was added to the reaction mixture, and then 50 mL of chloroform was added. The reaction mixture was transferred to a separatory funnel, and the organic layer was separated. Then the aqueous layer was extracted with 50 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula (yield amount 2.417 g, yield 92.8%).

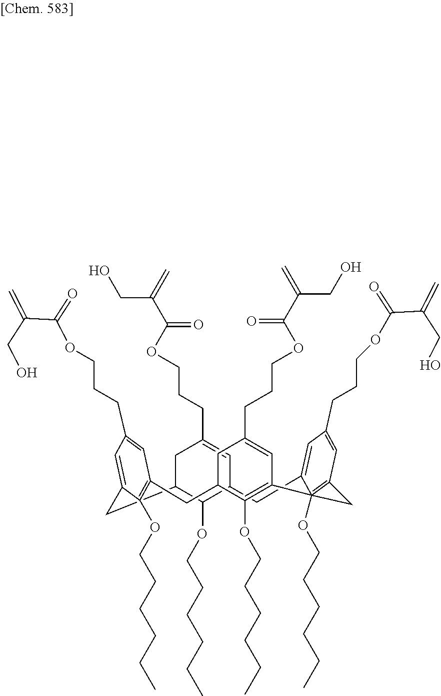

### Synthesis Example 80

The same procedure as in Synthesis Example 79 was repeated except that the compound obtained in Synthesis Example 75 (3.50 g, 2.32 mmol) was used instead of the compound obtained in Synthesis Example 74, to thereby obtain a compound represented by the following formula (2.214 g, yield 90.8%).

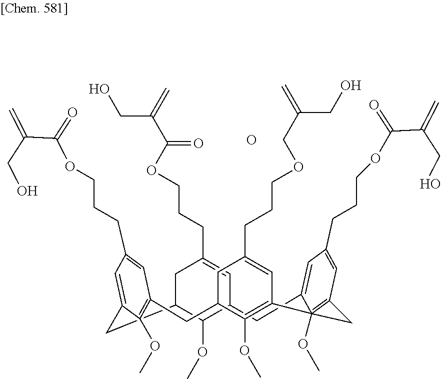

The same procedure as in Synthesis Example 79 was repeated except that the compound obtained in Synthesis Example 76 (3.50 g, 2.32 mmol) was used instead of the compound obtained in Synthesis Example 74, to thereby obtain a compound represented by the following formula (2.344 g, yield 92.1%).

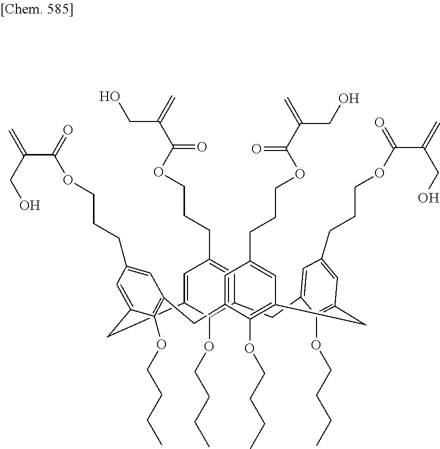 [SMILES: C=C(CO)C(=O)OCCCc1cc2c(OCCCC)c(c1)Cc1cc(CCCOC(=O)C(=C)CO)cc(c1OCCCC)Cc1cc(CCCOC(=O)C(=C)CO)cc(c1OCCCC)Cc1cc(CCCOC(=O)C(=C)CO)cc(c1OCCCC)C2]

### Synthesis Example 82

The same procedure as in Synthesis Example 79 was repeated except that the compound obtained in Synthesis Example 77 (3.50 g, 2.32 mmol) was used instead of the compound obtained in Synthesis Example 74, to thereby obtain a compound represented by the following formula (2.466 g, yield 93.7%).

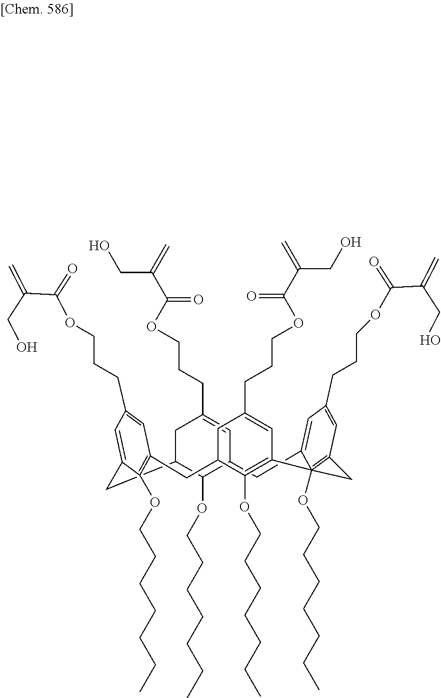

### Synthesis Example 83

The same procedure as in Synthesis Example 79 was repeated except that the compound obtained in Synthesis Example 78 (3.50 g, 1.42 mmol) was used instead of the compound obtained in Synthesis Example 74, to thereby obtain a compound represented by the following formula (2.608 g, yield 91.5%).

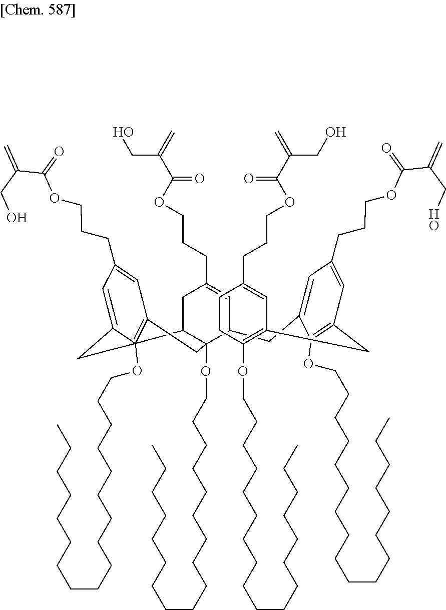

### Example 46

A 100 mL four-necked flask equipped with a stirrer, a dropping funnel, and a thermometer was charged, in a nitrogen atmosphere, with the compound obtained in Synthesis Example 79 (2.0 g, 1.50 mmol), triethylamine (1.218 g, 12.0 mmol), and methylene chloride (19 mL), and the mixture was stirred under cooling with ice. A solution prepared by dissolving methyl oxal chloride (1.105 g, 9.02 mmol) in methylene chloride (3 mL) was slowly added dropwise. After completion of the dropwise addition, the resulting mixture was stirred at room temperature for 8 hours. Water was added to the reaction mixture, and the resulting mixture was extracted with chloroform (40 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator, and the obtained yellow viscous liquid was purified by silica gel column chromatography to thereby obtain the target compound 010-6 (yield amount 2.175 g, yield 86.4%).

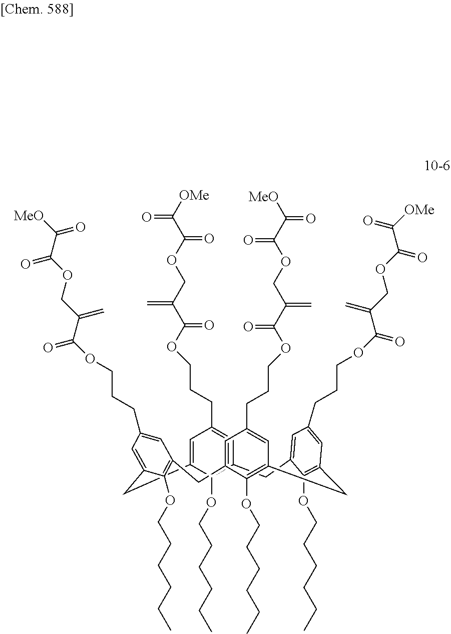 [SMILES: C=C(COC(=O)C(=O)OC)C(=O)OCCCc1cc2c(OCCCCCC)c(c1)Cc1cc(CCCOC(=O)C(=C)COC(=O)C(=O)OC)cc(c1OCCCCCC)Cc1cc(CCCOC(=O)C(=C)COC(=O)C(=O)OC)cc(c1OCCCCCC)Cc1cc(CCCOC(=O)C(=C)COC(=O)C(=O)OC)cc(c1OCCCCCC)C2]

### Example 47

The same procedure as in Example 46 was repeated except that the compound obtained in Synthesis Example 80 (2.00 g, 1.91 mmol) was used instead of the compound obtained in Synthesis Example 79, to thereby obtain the target compound 010-1 (2.191 g, yield 82.5%).

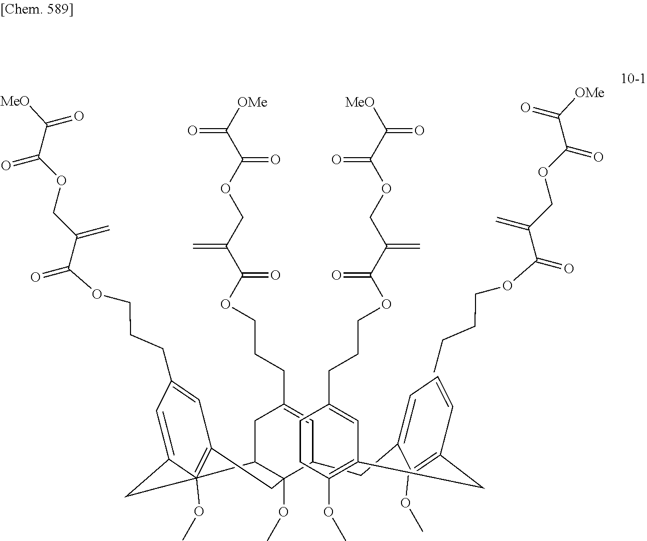

### Example 48

The same procedure as in Example 46 was repeated except that compound obtained in Synthesis Example 81 (2.00 g, 1.64 mmol) was used instead of the compound obtained in Synthesis Example 79, to thereby obtain the target compound 010-4 (2.140 g, yield 83.4%).

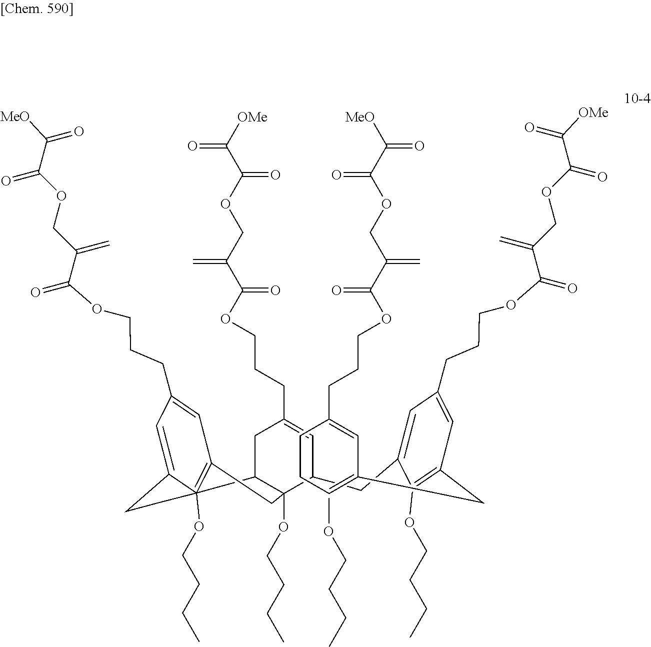

### Example 49

The same procedure as in Example 46 was repeated except that the compound obtained in Synthesis Example 82 (2.00 g, 1.44 mmol) was used instead of the compound obtained in Synthesis Example 79, to thereby obtain the target compound 010-7 (2.237 g, yield 89.6%).

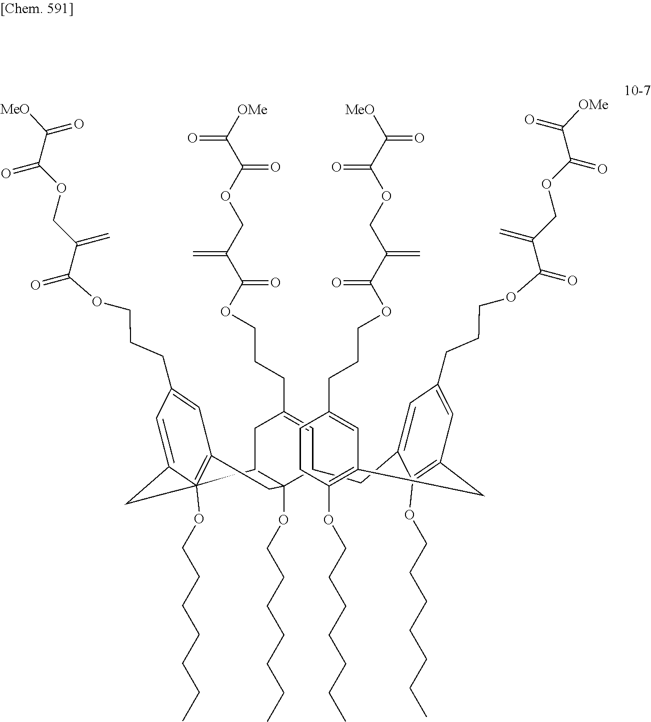

### Example 50

The same procedure as in Example 46 was repeated except that the compound obtained in Synthesis Example 83 (2.00 g, 1.00 mmol) was used instead of the compound obtained in Synthesis Example 79, to thereby obtain the target compound 010-18 (1.880 g, yield 80.2%).

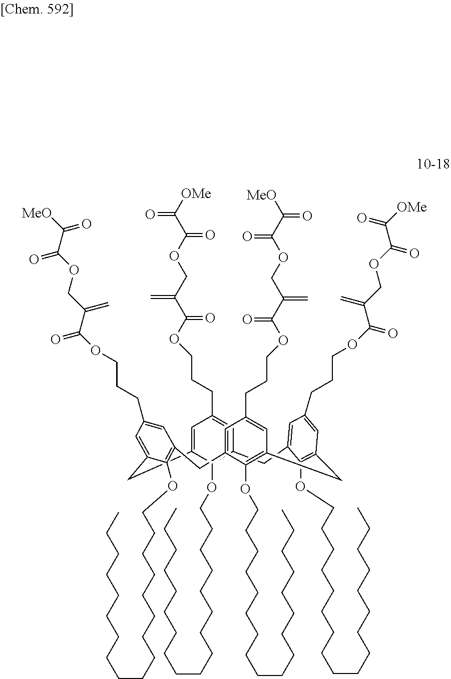 [SMILES: C=C(COC(=O)C(=O)OC)C(=O)OCCCc1cc2c(OCCCCCCCCCCCCCCCCCC)c(c1)Cc1cc(CCCOC(=O)C(=C)COC(=O)C(=O)OC)cc(c1OCCCCCCCCCCCCCCCCCC)Cc1cc(CCCOC(=O)C(=C)COC(=O)C(=O)OC)cc(c1OCCCCCCCCCCCCCCCCCC)Cc1cc(CCCOC(=O)C(=C)COC(=O)C(=O)OC)cc(c1OCCCCCCCCCCCCCCCCCC)C2]

### COMPARATIVE EXAMPLE

A 100 mL four-necked flask equipped with a stirrer, a thermometer, and a reflux condenser tube was charged with 1.00 g (1.212 mmol) of the compound obtained in Synthesis Example 20, 10.00 g of tetrahydrofuran, 1.907 g (7.271 mmol) of triphenylphosphine, and 0.6260 g (7.271 mmol) of methacrylic acid, and the mixture was stirred. A light yellow transparent solution. Then 1.470 g (7.271 mmol) of diisopropyl azodicarboxylate was added dropwise to the mixture placed in an ice bath over 30 minutes. A light yellow transparent solution. The light yellow transparent solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting orange viscus liquid was subjected to column chromatography (eluent: n-hexane:acetone=90:10) to thereby obtain compound (1′) represented by the following formula. Compound (1′) was vacuum dried (at 60° C. for 6 hours or longer). 0.9058 g, and the yield was 68.1%.

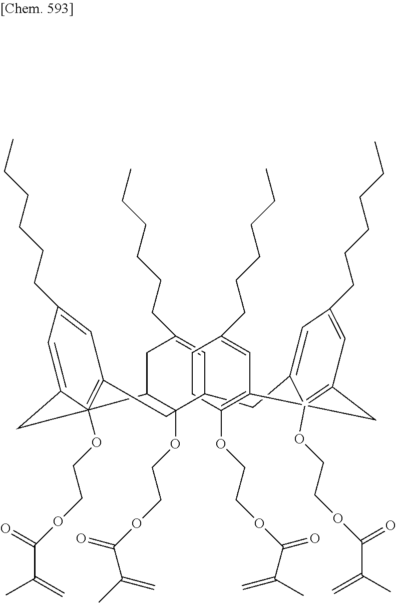

<Production of Curable Compositions>

0.25 g of one of the obtained calixarene compounds, 0.25 g of dipentaerythritol hexaacrylate (“A-DPH” manufactured by Shin Nakamura Chemical Co., Ltd.), 0.005 g of a polymerization initiator (“Irgacure 369” manufactured by BASF), and 9.5 g of propylene glycol monomethyl ether acetate were mixed to obtain a curable composition.

<Production of Layered Bodies>

The curable composition was applied to substrates 1 to 4 below by a spin coating method such that the thickness of the coating after curing was about 0.5 μm and then dried on a hot plate at 100° C. for 2 minutes. A high-pressure mercury lamp was used to irradiate the curable composition with UV rays at 500 mJ/cm in a nitrogen atmosphere to cure the curable composition, and layered bodies were thereby obtained.

Substrate 1: polymethyl methacrylate resin plate

Substrate 2: aluminum plate

Substrate 3: polyethylene terephthalate film having a SiO₂ thin layer (thickness 100 nm) (the curable composition was applied to the SiO₂ thin film)

<Evaluation of Adhesion>

A layered body stored in a 23° C. and 50% RH environment for 24 hours was used, and the adhesion was evaluated according to JIS K6500-5-6 (adhesive strength: a cross-cut method). A cellophane tape used was “CT-24” manufactured by Nichiban Co., Ltd. The criteria for the evaluation are as follows.

A: 80 or more out of 100 squares were not detached and remained present.

B: 50 to 79 out of 100 squares were not detached and remained present.

C: The number of squares that were not detached and remained present was 49 or less out of 100 squares.

<Evaluation of Moist Heat Resistance>

One of the curable compositions was applied to a 5 inch SiO substrate to a film thickness of about 50 μm using an applicator and dried on a hot plate at 100° C. for 2 minutes. A mask having an LS pattern with L/S=50 μm/50 μm was brought into tight contact with the coating obtained, and a high-pressure mercury lamp was used to irradiate the composition with UV rays at 1000 mJ/cm² in a nitrogen atmosphere to cure the composition. The substrate exposed to the light was developed using ethyl acetate to obtain an evaluation substrate. The obtained substrate was stored in a thermo-hygrostat at 85° C. and 85% RH for 100 hours, and a laser microscope (“VK-X200” manufactured by KEYENCE CORPORATION) was used to check the state of the pattern after a lapse of 100 hours. The criteria for the evaluation are as follows.

A: The entire pattern was well modified and maintained.

B: Cracking or chipping was observed in part of the pattern.

C: Cracking or chipping was observed in the pattern, and delamination of the pattern was also observed.

TABLE 24

| Calixarene compound | | 1-6 | 2-6 | 3-6 | 4-6 | 1-4 | 2-4 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 3-4 | 4-4 | 1-7 | 2-7 | 3-7 | 4-7 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 5-6 | 6-6 | 7-6 | 8-6 | 9-6 | 10-6 |
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 25

| Calixarene compound | | 11-6 | 12-6 | 13-6 | 14-6 | 15-6 | 16-6 |
|---|---|---|---|---|---|---|---|
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 17-6 | 18-6 | 19-6 | 20-6 | 21-6 | 22-6 |
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | B |
| Calixarene compound | | 23-6 | 24-6 | 25-6 | 26-6 | 27-6 | 28-6 |
| Adhesion | Substrate 1 | A | B | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 26

| Calixarene compound | | 29-6 | 30-6 | 31-6 | 32-6 | 33-6 | 34-6 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 35-6 | 36-6 | 33-4 | 34-4 | 35-4 | 36-4 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 33-7 | 34-7 | 35-7 | 36-7 | 33-18 | 34-18 |
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 27

| Calixarene compound | | 35-18 | 36-18 | 33-1 | 34-1 | 35-1 | 36-1 |
|---|---|---|---|---|---|---|---|
| Adhesion | Substrate 1 | A | B | A | A | B | B |
| | Substrate 2 | A | A | A | A | A | A |
| | Substrate 3 | A | A | A | A | A | A |
| Moist heat resistance | | A | A | A | A | A | A |
| Calixarene compound | | 37-6 | 38-6 | 39-6 | 40-6 | 41-6 | 42-6 |
| Adhesion | Substrate 1 | A | A | A | B | A | B |
| | Substrate 2 | A | A | A | A | A | A |
| | Substrate 3 | A | A | A | A | A | A |
| Moist heat resistance | | A | A | B | A | A | A |
| Calixarene compound | | 43-6 | 44-6 | 45-6 | 46-6 | 47-6 | 48-6 |
| Adhesion | Substrate 1 | A | B | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 28

| Calixarene compound | | 49-6 | 50-6 | 51-6 | 52-6 | 53-6 | 54-6 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 55-6 | 56-6 | 57-6 | 58-6 | 59-6 | 60-6 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 61-6 | 62-6 | 63-6 | 64-6 | 65-6 | 65-4 |
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 29

| Calixarene compound | | 65-7 | 65-18 | 65-1 | 66-6 | 67-6 | 68-6 |
|---|---|---|---|---|---|---|---|
| Adhesion | Substrate 1 | A | A | A | A | A | A |
| | Substrate 2 | A | B | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 01-6 | 02&03-6 | 04-6 | 01-1 | 02&03-1 | 04-1 |
|---|---|---|---|---|---|---|---|
| Adhesion | Substrate 1 | A | A | B | A | A | B |
| | Substrate 2 | A | A | A | A | A | A |
| | Substrate 3 | A | A | A | A | A | A |
| Moist heat resistance | | A | A | A | A | A | A |
| Calixarene compound | | 01-4 | 02&03-4 | 04-4 | 01-7 | 02&03-7 | 04-7 |
| Adhesion | Substrate 1 | A | A | B | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 30

| Calixarene compound | | 01-18 | 02&03-18 | 04-18 | 05-6 | 05-1 | 05-4 |
|---|---|---|---|---|---|---|---|
| Adhesion | Substrate 1 | A | A | B | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 05-7 | 05-18 | 06-6 | 07&08-6 | 09-6 | 06-1 |
|---|---|---|---|---|---|---|---|
| Adhesion | Substrate 1 | A | A | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 07&08-1 | 09-1 | 06-4 | 07&08-4 | 09-4 | 06-7 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 31

| Calixarene compound | | 07&08-7 | 09-7 | 06-18 | 07&08-18 | 09-18 | 010-6 |
|---|---|---|---|---|---|---|---|
| Adhesion | Substrate 1 | A | 8 | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 010-1 | 010-4 | 010-7 | 010-18 | 1′ |
|---|---|---|---|---|---|---|
| Adhesion | Substrate 1 | A | A | A | A | C |
| | Substrate 2 | A | A | A | A | C |
| | Substrate 3 | A | A | A | A | C |

| | | | | | |
|---|---|---|---|---|---|
| Moist heat resistance | A | A | A | A | B |

### Example Group <V>

### Synthesis Example 1

A 20 L separable four-necked flask equipped with a stirrer, a thermometer, and a reflux condenser tube was quickly charged with 1000 g (1.54 mol) of t-butyl-calix[4]arene, 1159 g (12.32 mol) of phenol, and 9375 mL of dehydrated toluene, and the mixture was stirred at 300 rpm under nitrogen flow. The t-butyl-calix[4]arene used as a raw material was not dissolved but was suspended. Then, while the flask was cooled in an ice bath, and 1643 g (12.32 mol) of anhydrous aluminum chloride (III) was added in several portions. The solution turned into a light orange transparent solution, and the anhydrous aluminum chloride (III) precipitated on the bottom. The mixture was allowed to react at room temperature for 5 hours. Then the contents were transferred to a 1 L beaker, and 20 kg of ice, 10 L of 1N hydrochloric acid, and 20 L of chloroform were added to stop the reaction. The mixture turned into a light yellow transparent solution. This reaction mixture was transferred to a separatory funnel, and the organic layer was separated. Then the aqueous layer was extracted with 5 L of chloroform three times, and the extract was combined with the organic layer. The organic layer was pre-dried over anhydrous magnesium sulfate and filtered. The solvent was removed by evaporation using an evaporator, and a mixture of white crystals and a colorless transparent solution was thereby obtained. Methanol was added slowly to the mixture under stirring to reprecipitate the crystals. The white crystals were filtered on a Kiriyama funnel and washed with methanol. The obtained white crystals were vacuum dried (at 50° C. for 6 hours or longer) to thereby obtain 597 g of the target intermediate A. The yield was 91%.

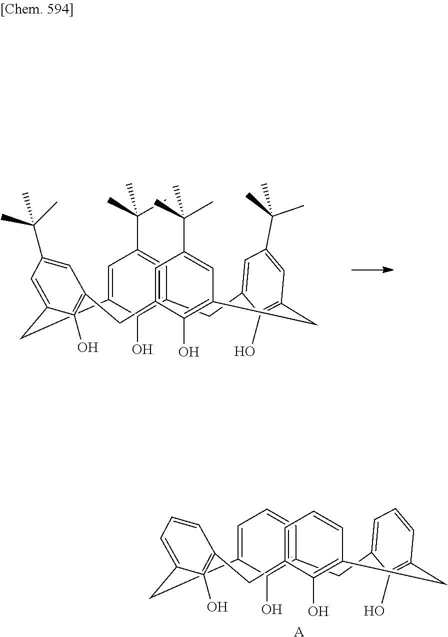

### Synthesis Example 2

A 2 L four-necked flask equipped with a stirrer, a thermometer, and a reflux condenser tube was charged with 205 g (1.52 mol) of n-hexanoyl chloride and 709 g of nitroethane, and the mixture was stirred. Then, while the flask was cooled in an ice bath, 243 g (1.82 mol) of anhydrous aluminum chloride (III) was added in several portions. The solution turned into a light orange transparent solution. The resulting solution was stirred at room temperature for 30 minutes, and 100 g (0.236 mol) of intermediate A was added in several portions. The mixture foamed and turned into an orange transparent solution. The solution was allowed to react at room temperature for 5 hours. Then the contents were slowly transferred to a 2 L beaker containing 450 mL of chloroform and 956 g of ice water to stop the reaction. Then 1N hydrochloric acid was added until the pH was 1. The reaction mixture was transferred to a separatory funnel, and the organic layer was separated. Next, the aqueous layer was extracted with 400 mL of chloroform three times, and the extract was combined with the organic layer. The organic layer was pre-dried over anhydrous magnesium sulfate. The solvent was removed by evaporation using an evaporator, and a yellow transparent solution was thereby obtained. While the solution was cooled in an ice bath, methanol was added to reprecipitate crystals. The white crystals were filtered on a Kiriyama funnel and recrystallized with chloroform and methanol. The obtained white crystals were vacuum dried (at 60° C. for 6 hours or longer) to thereby obtain 122 g of compound B-6 represented by the following structural formula. The yield was 63%.

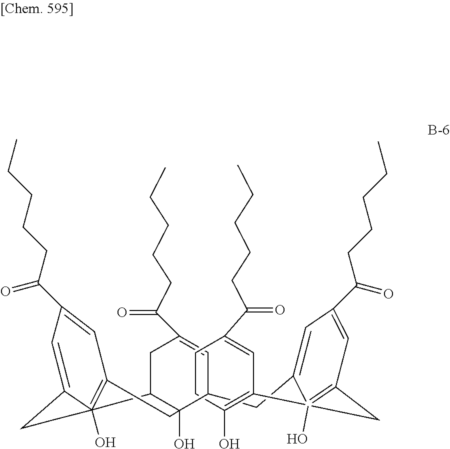

### Synthesis Example 3

The same procedure as in Synthesis Example 2 was repeated except that butyl chloride was used instead of n-hexanoyl chloride, to thereby obtain 106 g of compound B-4 represented by the following structural formula. The yield was 64%.

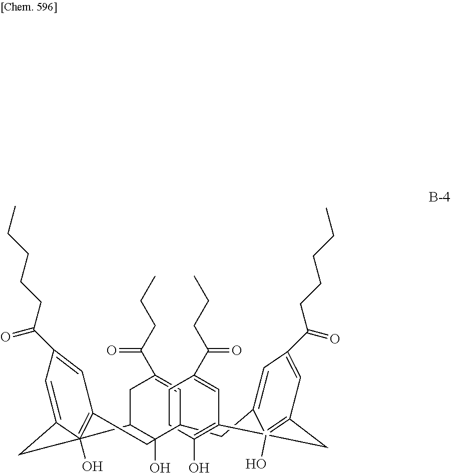

### Synthesis Example 4

The same procedure as in Synthesis Example 2 was repeated except that n-heptanoyl chloride was used instead of n-hexanoyl chloride, to thereby obtain 134 g of compound B-7 represented by the following structural formula. The yield was 65%.

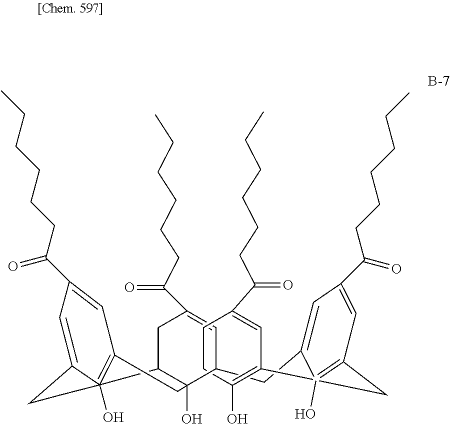

### Synthesis Example 5

The same procedure as in Synthesis Example 2 was repeated except that stearoyl chloride was used instead of n-hexanoyl chloride, to thereby obtain 228 g of compound B-18 represented by the following structural formula. The yield was 65%.

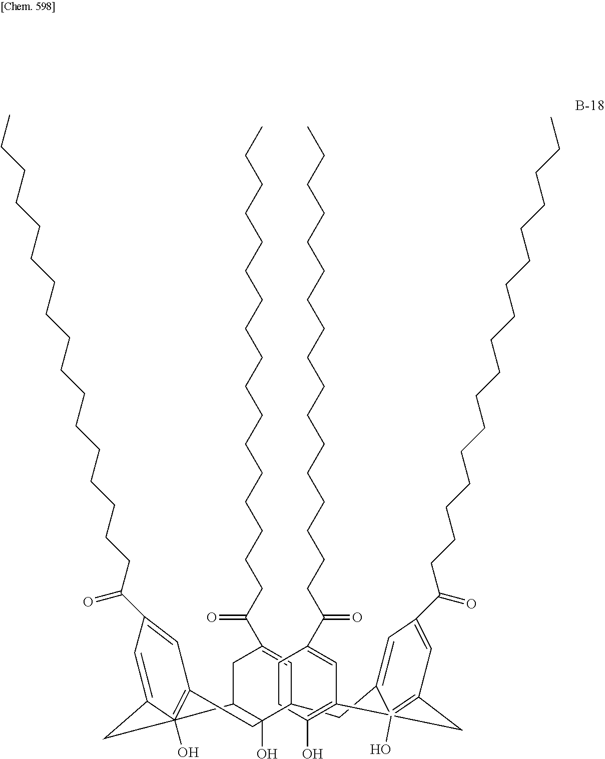

### Synthesis Example 6

A 100 mL four-necked flask equipped with a stirrer, a thermometer, and a reflux condenser tube was charged with 5.00 g (6.119 mmol) of B-6, 17.0 g of acetonitrile, 11.28 g (48.95 mmol) of potassium carbonate, 0.813 g (4.896 mmol) of potassium iodide, and 7.489 g (48.95 mmol) of methyl 2-bromoacetate, and the mixture was allowed to react at 70° C. for 24 hours. The resulting mixture was cooled to room temperature, and ion exchanged water and 0.3N hydrochloric acid were added until the pH was 6. Then 50 g of chloroform was added. The reaction mixture was transferred to a separatory funnel, and the organic layer was separated. Next, the aqueous layer was extracted with 50 g of chloroform three times, and the extract was combined with the organic layer. The organic layer was pre-dried over anhydrous magnesium sulfate, and the solvent was removed by evaporation using an evaporator to thereby obtain a red waxy solid. The obtained red waxy solid was vacuum dried (at 60° C. for 6 hours or longer) to thereby obtain 5.04 g of compound C-6 represented by the following structural formula. The yield was 74.5%.

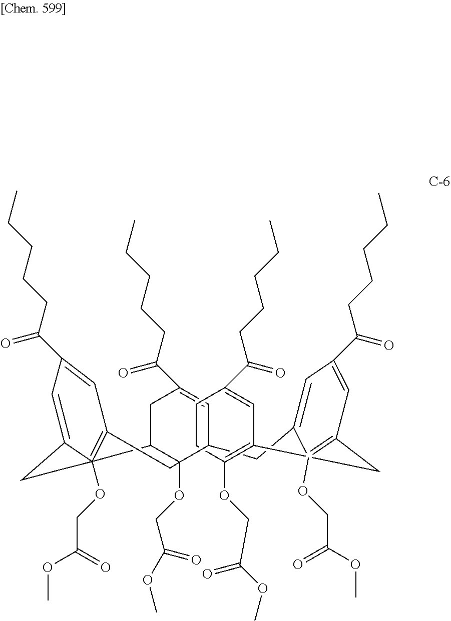

### Synthesis Example 7

The same procedure as in Synthesis Example 6 was repeated except that B-4 was used instead of B-6, to thereby obtain 4.88 g of compound C-4 represented by the following structural formula with a yield of 69.3%.

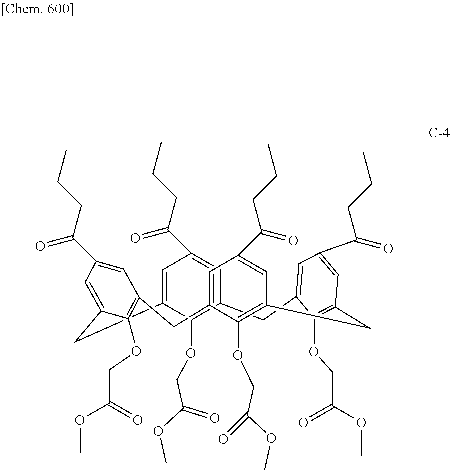 [SMILES: CCCC(=O)c1cc2c(OCC(=O)OC)c(c1)Cc1cc(C(=O)CCC)cc(c1OCC(=O)OC)Cc1cc(C(=O)CCC)cc(c1OCC(=O)OC)Cc1cc(C(=O)CCC)cc(c1OCC(=O)OC)C2]

### Synthesis Example 8

The same procedure as in Synthesis Example 6 was repeated except that B-7 was used instead of B-6, to thereby obtain 5.12 g of compound C-7 represented by the following structural formula with a yield of 77.0%.

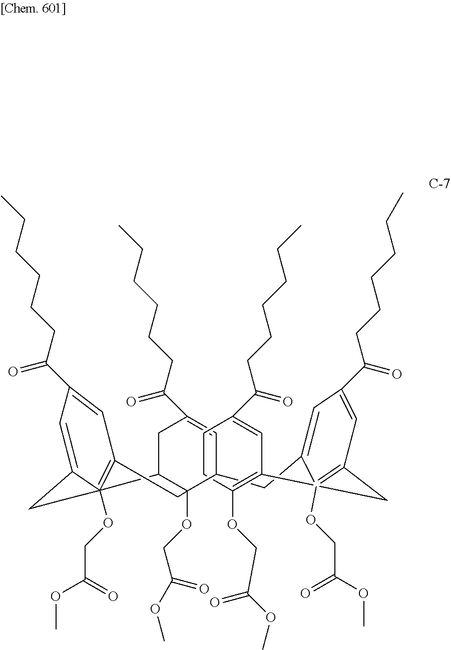

### Synthesis Example 9

The same procedure as in Synthesis Example 6 was repeated except that B-18 was used instead of B-6, to thereby obtain 5.34 g of compound C-18 represented by the following structural formula with a yield of 89.5%.

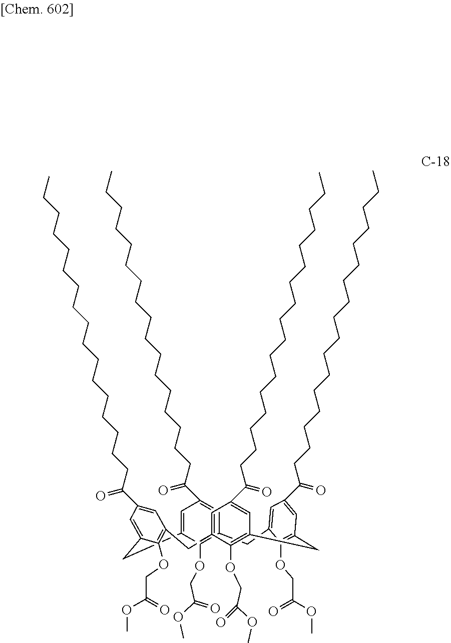 [SMILES: CCCCCCCCCCCCCCCCCC(=O)c1cc2c(OCC(=O)OC)c(c1)Cc1cc(C(=O)CCCCCCCCCCCCCCCCC)cc(c1OCC(=O)OC)Cc1cc(C(=O)CCCCCCCCCCCCCCCCC)cc(c1OCC(=O)OC)Cc1cc(C(=O)CCCCCCCCCCCCCCCCC)cc(c1OCC(=O)OC)C2]

### Synthesis Example 10

A 500 mL four-necked flask equipped with a stirrer, a thermometer, and a reflux condenser tube was charged, in an ice bath, with 16.44 g of tetrahydrofuran, and 1.038 g (27.35 mmol) of lithium aluminum hydride was slowly added. 5.04 g (4.559 mmol) of C-6 diluted with 49.31 g of tetrahydrofuran was added dropwise from a dropping funnel such that the temperature did not exceed 10° C. The resulting gray suspension reaction solution was allowed to react at room temperature for 6 hours. 30 g of chloroform was added to the reaction solution placed in an ice bath, and 30 g of 5N hydrochloric acid was added dropwise to stop the reaction. Then diatomaceous earth was used to filter the reaction solution, and the filtrate was transferred to a separatory funnel to separate the organic layer. Next, the aqueous layer was extracted with 30 g of chloroform three times, and the extract was combined with the organic layer. The organic layer was pre-dried over anhydrous magnesium sulfate, and the solvent was removed by evaporation using an evaporator to thereby obtain a light yellow liquid. By-products were removed by column chromatography using an eluent: n-hexane:ethyl acetate=1:1, and then the target compound was eluted with an eluent: chloroform:isopropyl alcohol=5:1. The eluent was removed by evaporation under reduced pressure to thereby obtain 2.857 g of white solid compound D-6 represented by the following structural formula. The yield was 63.1%.

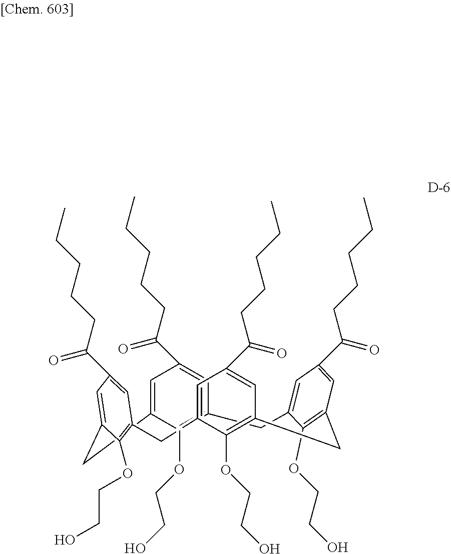 [SMILES: CCCCCC(=O)c1cc2c(OCCO)c(c1)Cc1cc(C(=O)CCCCC)cc(c1OCCO)Cc1cc(C(=O)CCCCC)cc(c1OCCO)Cc1cc(C(=O)CCCCC)cc(c1OCCO)C2]

### Synthesis Example 11

The same procedure as in Synthesis Example 10 was repeated except that C-4 was used instead of C-6, to thereby obtain 3.06 g of compound D-4 represented by the following structural formula with a yield of 69.0%.

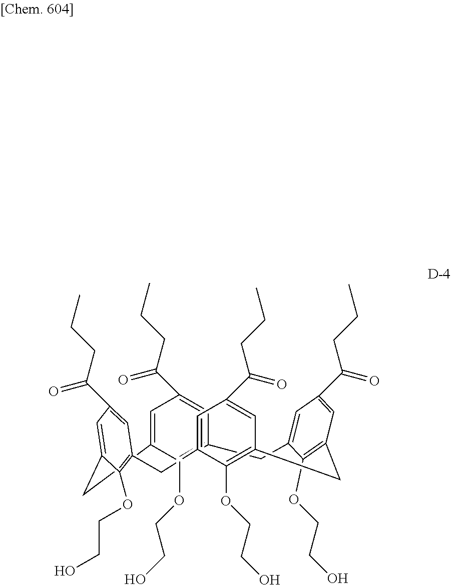 [SMILES: CCCC(=O)c1cc2c(OCCO)c(c1)Cc1cc(C(=O)CCC)cc(c1OCCO)Cc1cc(C(=O)CCC)cc(c1OCCO)Cc1cc(C(=O)CCC)cc(c1OCCO)C2]

### Synthesis Example 12

The same procedure as in Synthesis Example 10 was repeated except that C-7 was used instead of C-6, to thereby obtain 3.11 g of compound D-7 represented by the following structural formula with a yield of 68.2%.

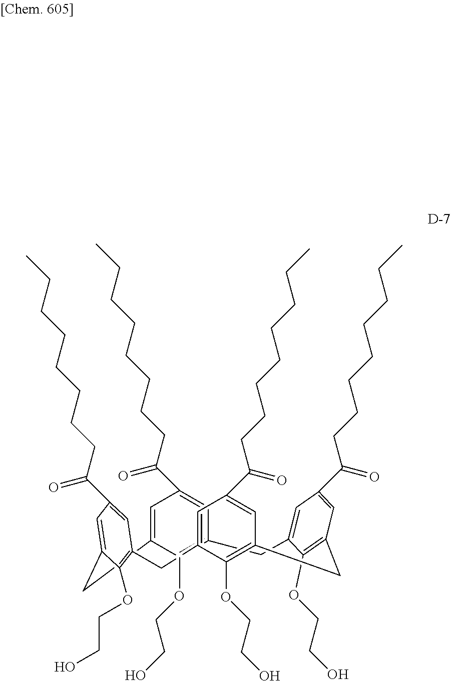 [SMILES: CCCCCCCCC(=O)c1cc2c(OCCO)c(c1)Cc1cc(C(=O)CCCCCCCC)cc(c1OCCO)Cc1cc(C(=O)CCCCCCCC)cc(c1OCCO)Cc1cc(C(=O)CCCCCCCC)cc(c1OCCO)C2]

### Example 1

A 50 mL four-necked flask equipped with a stirrer, a thermometer, and a reflux condenser tube was charged with 1.00 g (1.007 mmol) of D-6, 2.904 g of tetrahydrofuran, 2.112 g (8.054 mmol) of triphenylphosphine, 0.173 g (2.014 mmol) of methacrylic acid, and 0.713 g (6.041 mmol) of monomethyl malonate, and the mixture was stirred. An ocher suspension solution was obtained. Then 1.810 g (8.054 mmol) of diisopropyl azodicarboxylate diluted with 1.452 g of tetrahydrofuran was added dropwise to the suspension solution placed in an ice bath over 30 minutes. The resulting orange transparent reaction solution was stirred at room temperature for 10 hours. Hexane was added to the reaction solution to precipitate and remove by-products such as triphenylphosphine, and the resulting mixture was extracted with chloroform, washed with water and saturated brine, and dried over magnesium sulfate. The solvent was removed by evaporation using an evaporator, and the obtained red viscous liquid was purified by column chromatography (eluent: n-hexane:ethyl acetate=85:15) to thereby obtain 0.453 g of the target compound 1-6 with a yield of 33.0%, 0.231 g of the target compound 2-6 with a yield of 17.3%, 0.202 g of the target compound 3-6 with a yield of 15.1%, and 0.131 g of the target compound 4-6 with a yield of 10.0%.

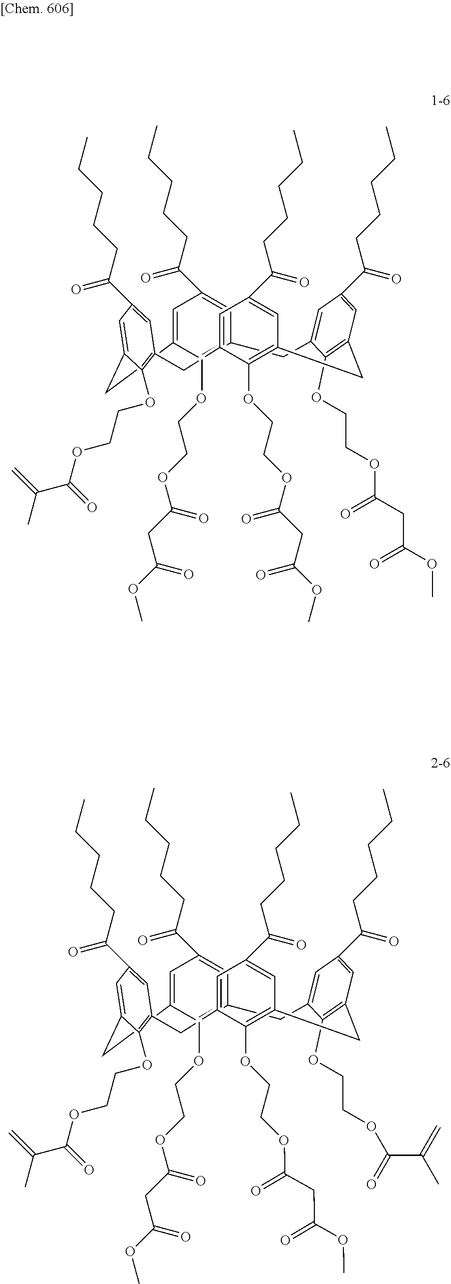

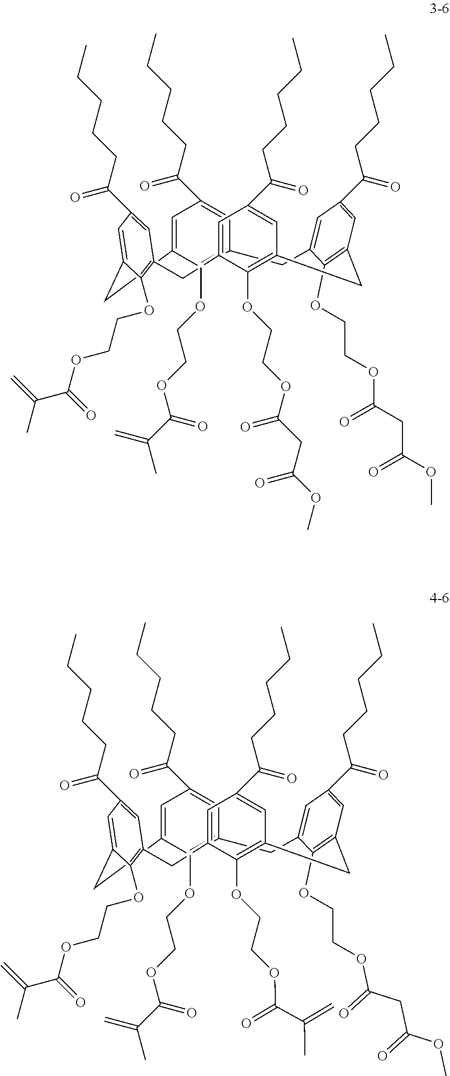

### Example 2

The same procedure as in Example 1 was repeated except that D-4 was used instead of D-6, to thereby obtain 0.437 g of the target compound 1-4 with a yield of 30.8%, 0.201 g of the target compound 2-4 with a yield of 14.5%, 0.198 g of the target compound 3-4 with a yield of 14.3%, and 0.142 g of the target compound 4-4 with a yield of 10.6%.

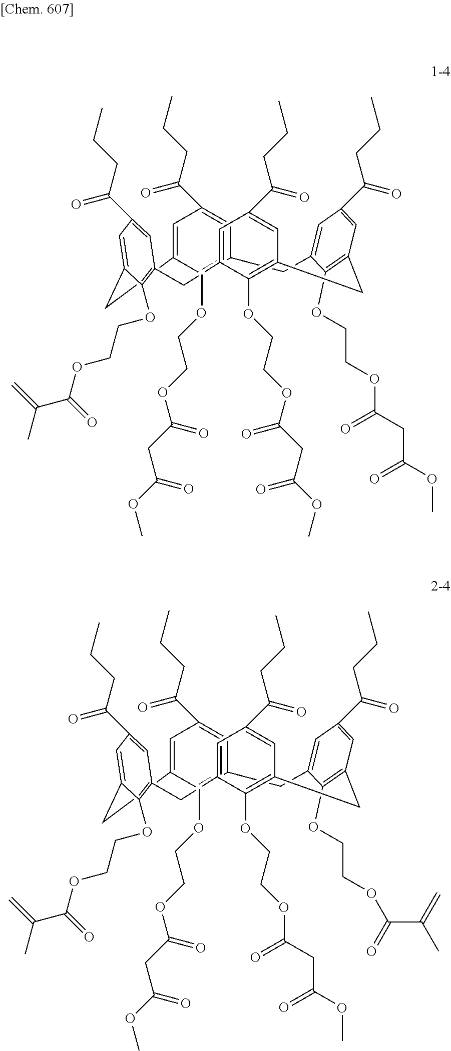

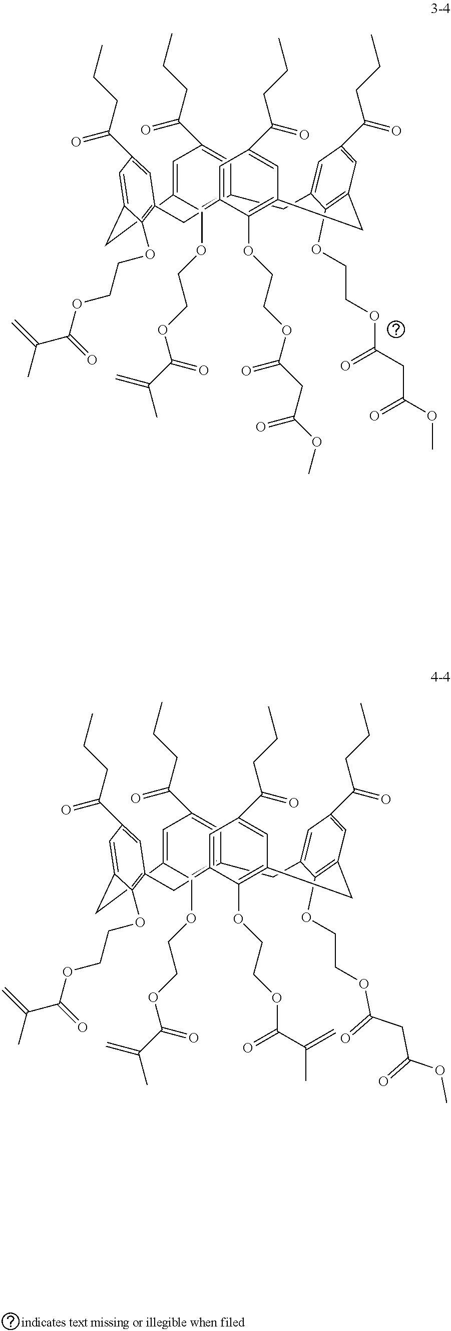

### Example 3

The same procedure as in Example 1 was repeated except that D-7 was used instead of D-6, to thereby obtain 0.468 g of the target compound 1-7 with a yield of 34.6%, 0.243 g of the target compound 2-7 with a yield of 18.4% 0.230 g of the target compound 3-7 with a yield of 17.4%, and 0.113 g of the target compound 4-7 with a yield of 8.76%.

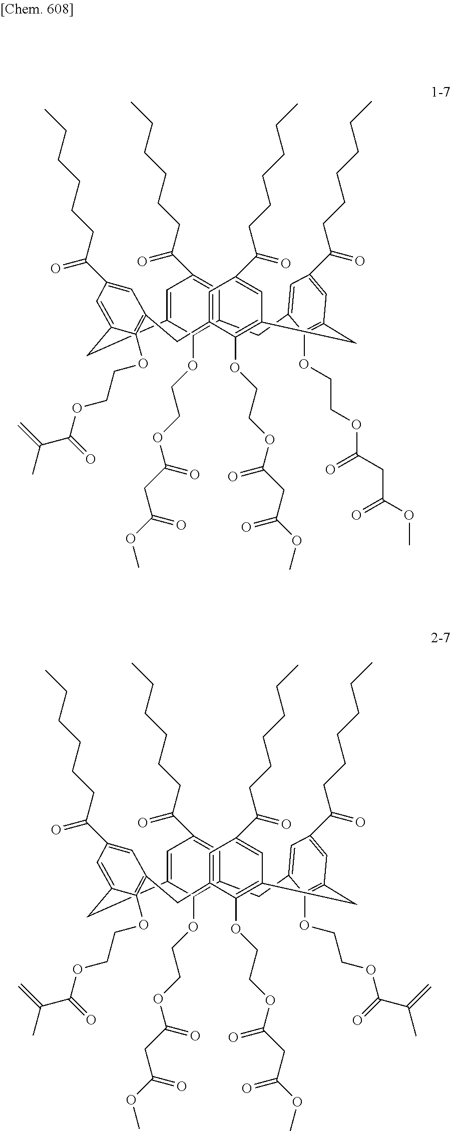

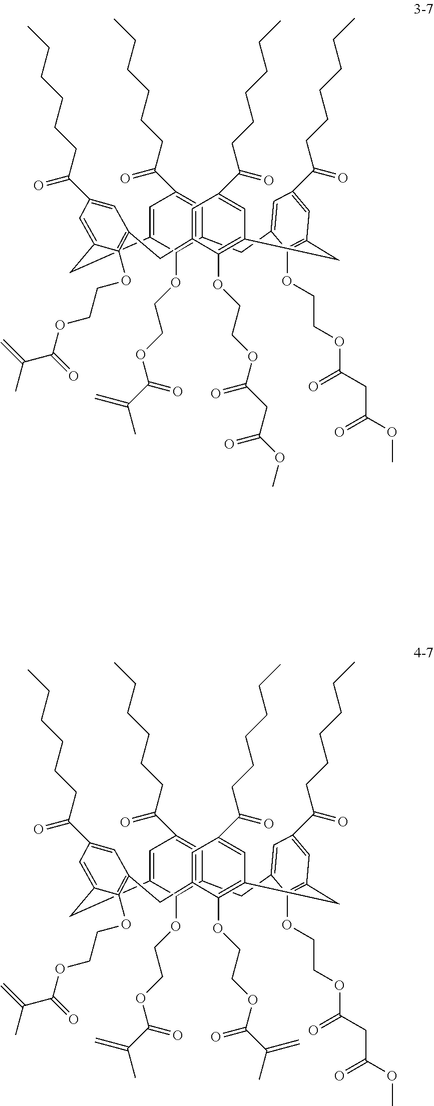

### Example 4

The same procedure as in Example 1 was repeated except that acrylic acid was used instead of methacrylic acid, to thereby obtain 0.439 g of the target compound 5-6 with a yield of 32.4%, 0.222 g of the target compound 6-6 with a yield of 16.9%, 0.197 g of the target compound 7-6 with a yield of 15.0%, and 0.145 g of the target compound 8-6 with a yield of 11.5%.

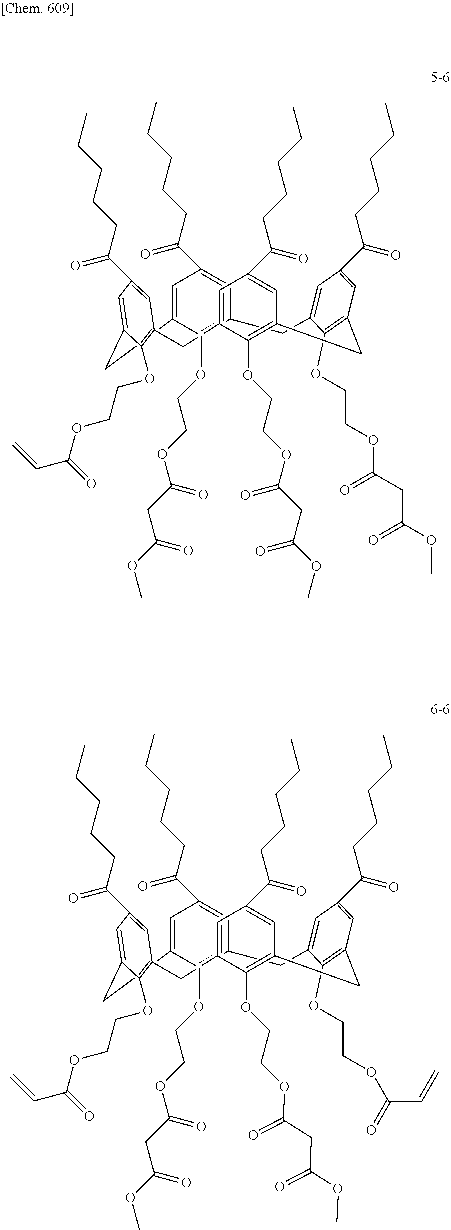

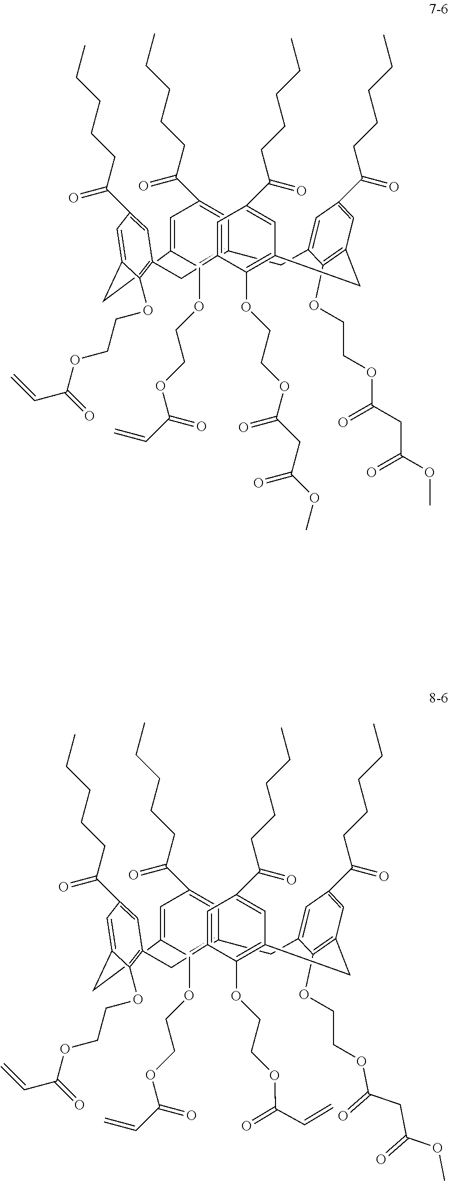

### Example 5

The same procedure as in Example 1 was repeated except that monoethyl malonate was used instead of monomethyl malonate, to thereby obtain 0.467 g of the target compound 9-6 with a yield of 33.0%, 0.234 g of the target compound 10-6 with a yield of 17.1%, 0.203 g of the target compound 11-6 with a yield of 14.9%, and 0.133 g of the target compound 12-6 with a yield of 10.1%.

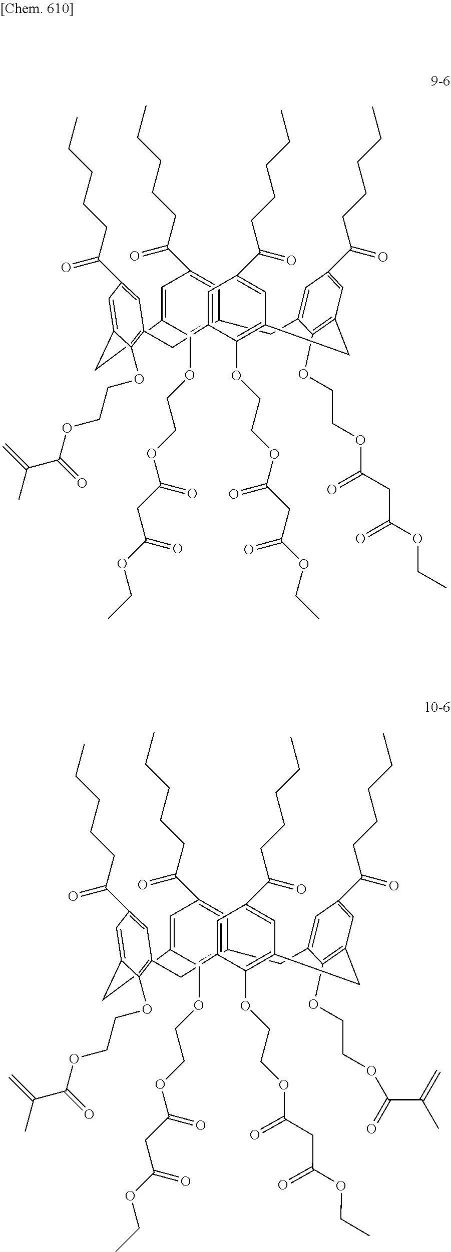

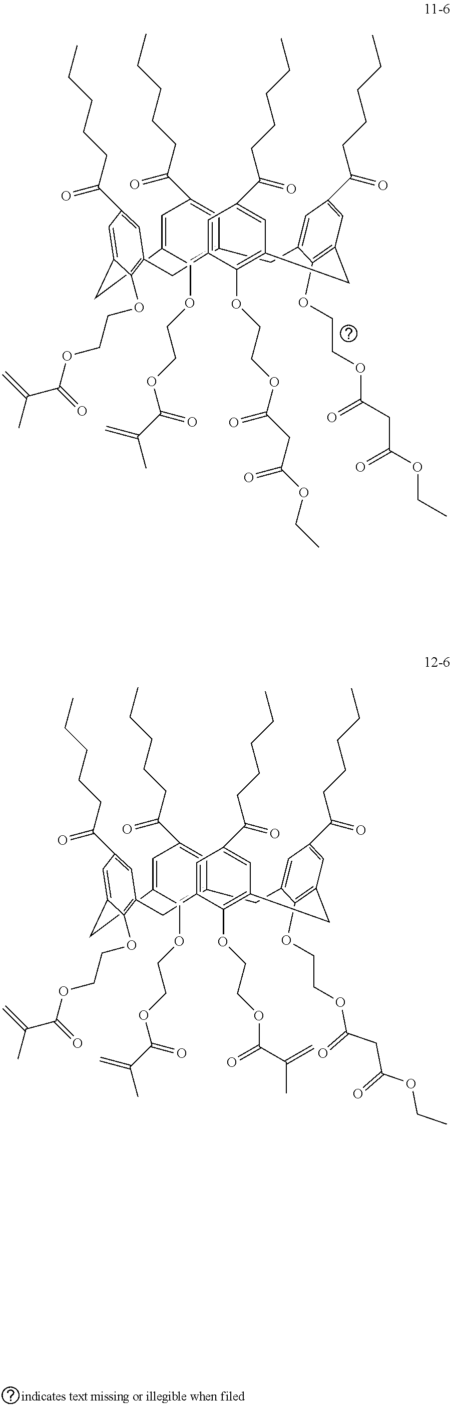

### Example 6

The same procedure as in Example 5 was repeated except that D-4 was used instead of D-6, to thereby obtain 0.467 g of the target compound 9-4 with a yield of 31.9%, 0.234 g of the target compound 10-4 with a yield of 16.6%, 0.203 g of the target compound 11-4 with a yield of 14.4%, and 0.133 g of the target compound 12-4 with a yield of 9.77%.

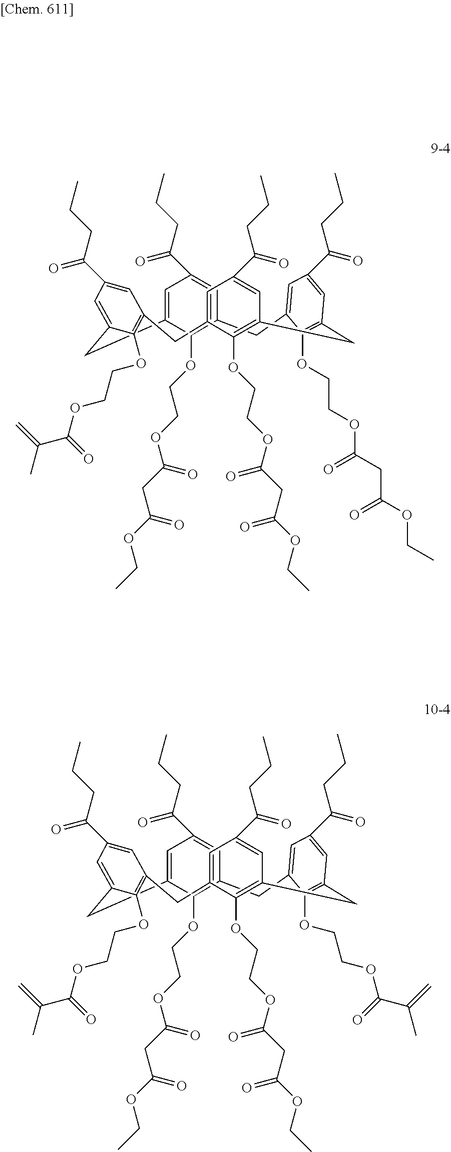

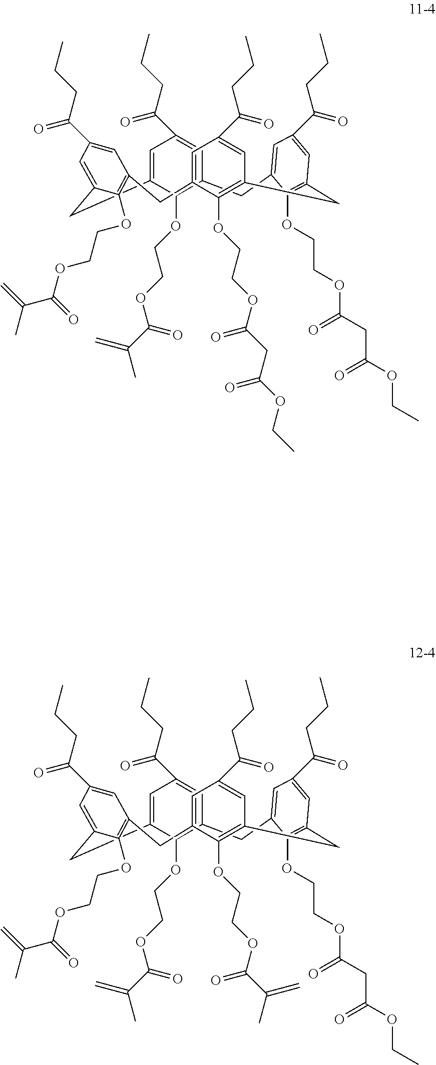

### Example 7

The same procedure as in Example 5 was repeated except that D-7 was used instead of D-6, to thereby obtain 0.467 g of the target compound 9-7 with a yield of 33.6%, 0.210 g of the target compound 10-7 with a yield of 15.6%, 0.228 g of the target compound 11-7 with a yield of 16.9%, and 0.176 g of the target compound 12-7 with a yield of 13.5%.

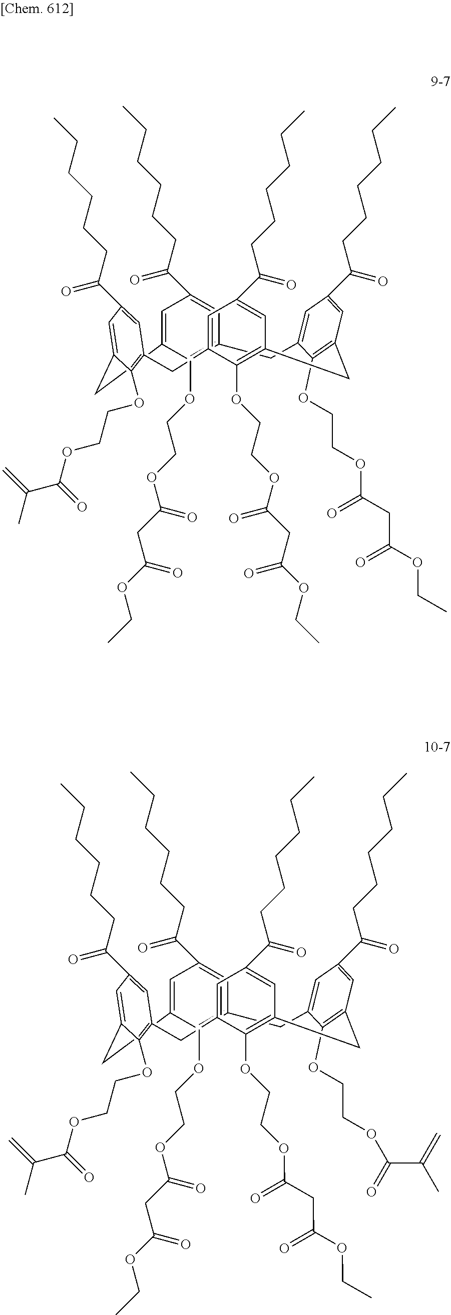

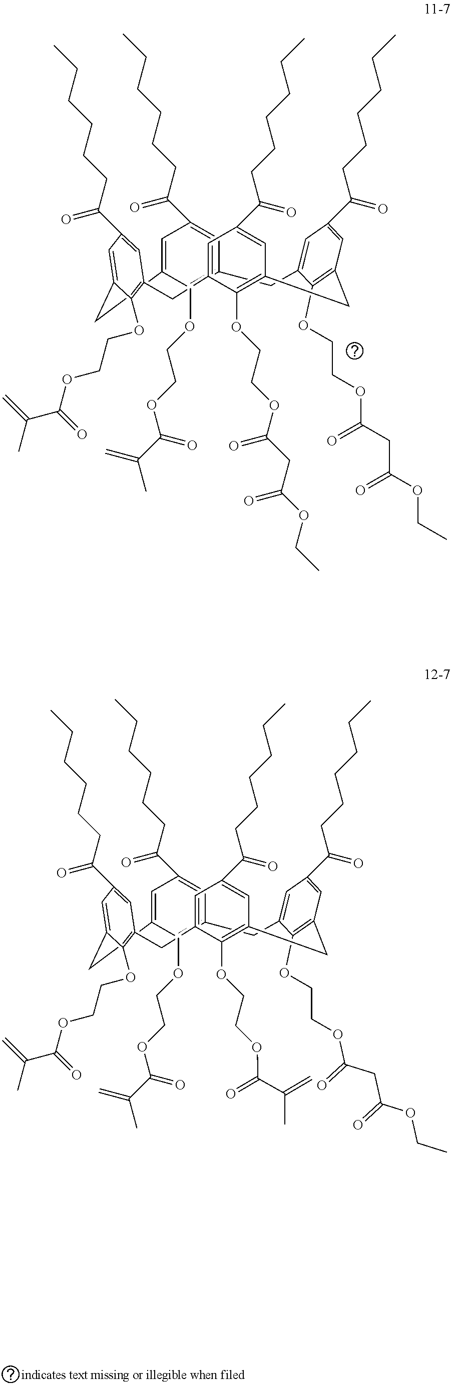

### Example 8

The same procedure as in Example 5 was repeated except that acrylic acid was used instead of methacrylic acid, to thereby obtain 0.409 g of the target compound 13-6 with a yield of 29.2%, 0.193 g of the target compound 14-6 with a yield of 14.4%, 0.189 g of the target compound 15-6 with a yield of 14.1%, and 0.124 g of the target compound 16-6 with a yield of 9.60%.

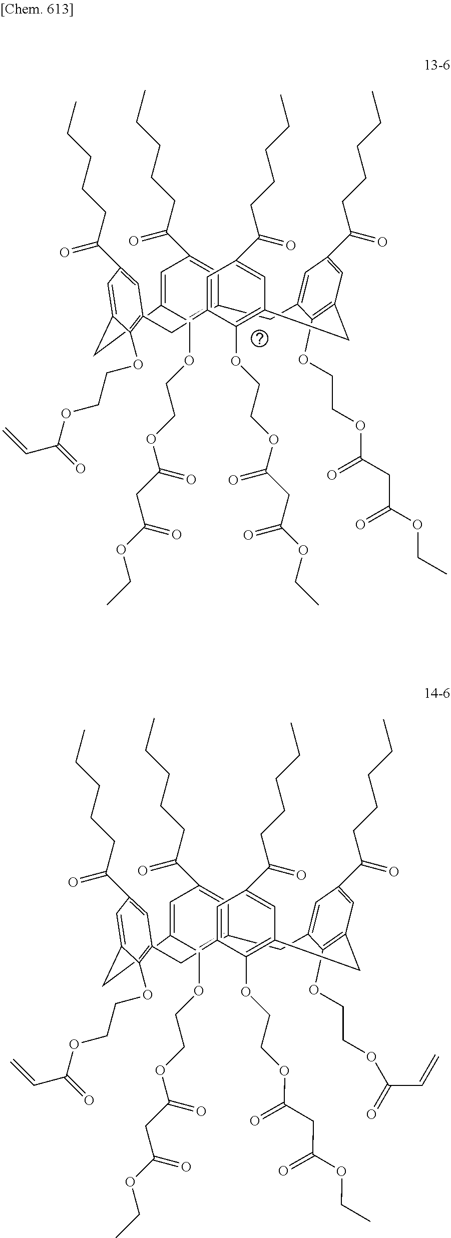

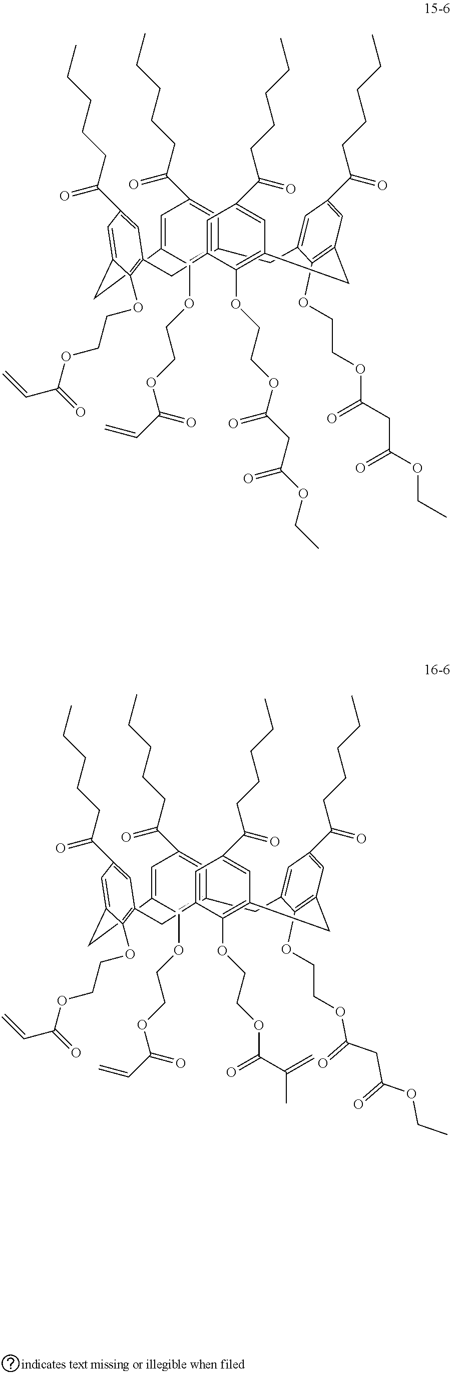

### Synthesis Example 13

The same procedure as in Synthesis Example 10 was repeated except that methyl bromopyopionate was used instead of methyl bromoacetate, to thereby obtain 4.307 g of compound E-6 represented by the following structural formula. The yield was 60.6%.

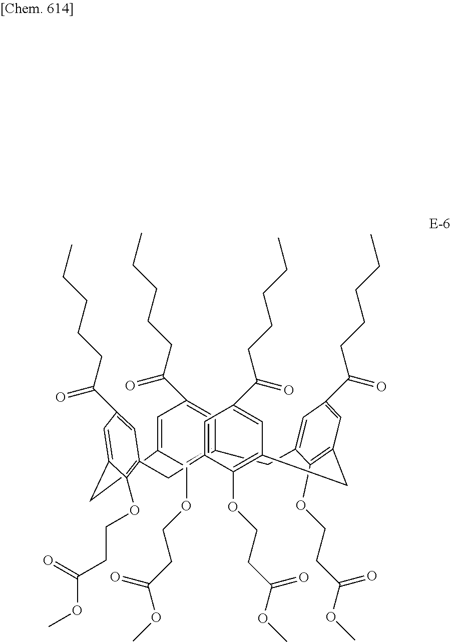 [SMILES: CCCCCC(=O)c1cc2c(OCCC(=O)OC)c(c1)Cc1cc(C(=O)CCCCC)cc(c1OCCC(=O)OC)Cc1cc(C(=O)CCCCC)cc(c1OCCC(=O)OC)Cc1cc(C(=O)CCCCC)cc(c1OCCC(=O)OC)C2]

### Synthesis Example 14

The same procedure as in Synthesis Example 11 was repeated except that E-6 was used instead of C-6, to thereby obtain 2.989 g of compound F-6 represented by the following structural formula. The yield was 80.6%.

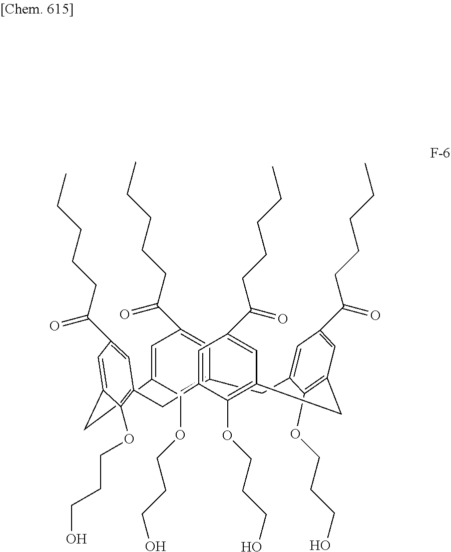 [SMILES: CCCCCC(=O)c1cc2c(OCCCO)c(c1)Cc1cc(C(=O)CCCCC)cc(c1OCCCO)Cc1cc(C(=O)CCCCC)cc(c1OCCCO)Cc1cc(C(=O)CCCCC)cc(c1OCCCO)C2]

### Example 9

The same procedure as in Example 1 was repeated except that F-6 was used instead of D-6, to thereby obtain 0.438 g of the target compound 17-6 with a yield of 32.4%, 0.214 g of the target compound 18-6 with a yield of 16.2%, 0.223 g of the target compound 19-6 with a yield of 16.9%, and 0.201 g of the target compound 20-6 with a yield of 15.6%.

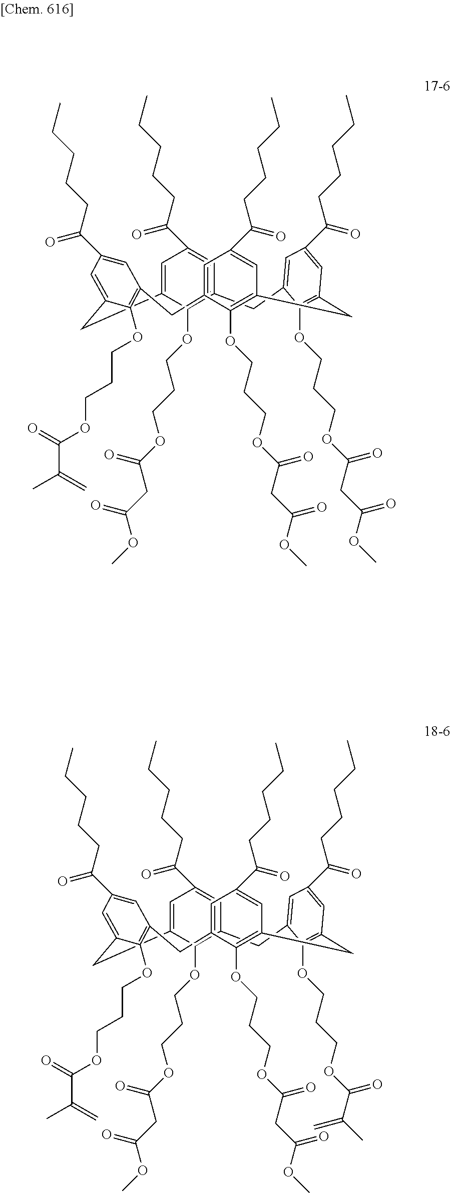 [SMILES: C=C(C)C(=O)OCCCOc1c2cc(C(=O)CCCCC)cc1Cc1cc(C(=O)CCCCC)cc(c1OCCCOC(=O)CC(=O)OC)Cc1cc(C(=O)CCCCC)cc(c1OCCCOC(=O)CC(=O)OC)Cc1cc(C(=O)CCCCC)cc(c1OCCCOC(=O)CC(=O)OC)C2 ; C=C(C)C(=O)OCCCOc1c2cc(C(=O)CCCCC)cc1Cc1cc(C(=O)CCCCC)cc(c1OCCCOC(=O)CC(=O)OC)Cc1cc(C(=O)CCCCC)cc(c1OCCCOC(=O)C(=C)C)Cc1cc(C(=O)CCCCC)cc(c1OCCCOC(=O)CC(=O)OC)C2]

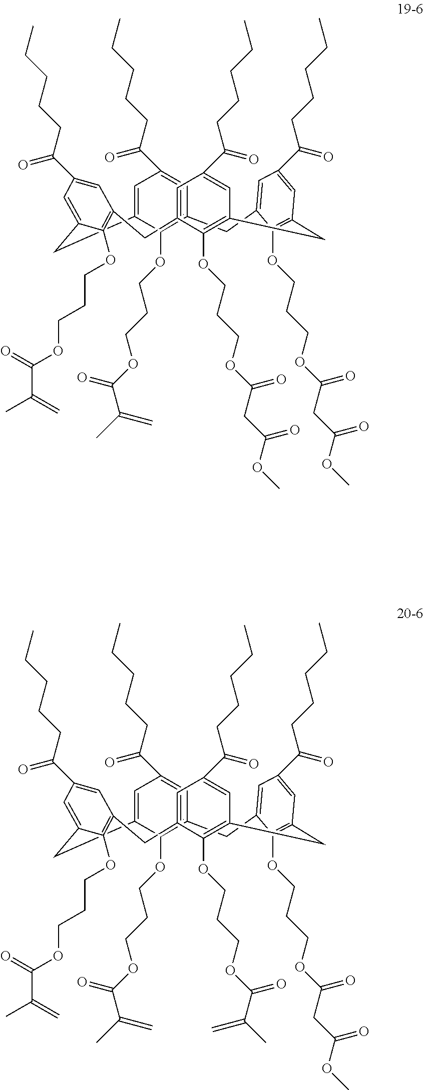 [SMILES: C=C(C)C(=O)OCCCOc1c2cc(C(=O)CCCCC)cc1Cc1cc(C(=O)CCCCC)cc(c1OCCCOC(=O)C(=C)C)Cc1cc(C(=O)CCCCC)cc(c1OCCCOC(=O)CC(=O)OC)Cc1cc(C(=O)CCCCC)cc(c1OCCCOC(=O)CC(=O)OC)C2 ; C=C(C)C(=O)OCCCOc1c2cc(C(=O)CCCCC)cc1Cc1cc(C(=O)CCCCC)cc(c1OCCCOC(=O)C(=C)C)Cc1cc(C(=O)CCCCC)cc(c1OCCCOC(=O)C(=C)C)Cc1cc(C(=O)CCCCC)cc(c1OCCCOC(=O)CC(=O)OC)C2]

### Example 10

The same procedure as in Example 9 was repeated except that acrylic acid was used instead of methacrylic acid, to thereby obtain 0.420 g of the target compound 21-6 with a yield of 31.4%, 0.206 g of the target compound 22-6 with a yield of 15.9%, 0.219 g of the target compound 23-6 with a yield of 16.9%, and 0.137 g of the target compound 24-6 with a yield of 11.0%.

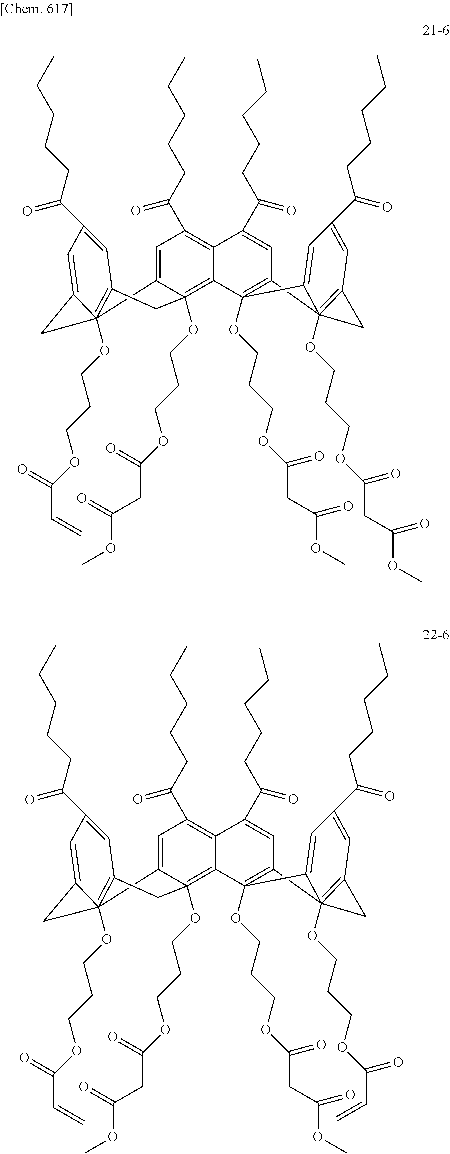

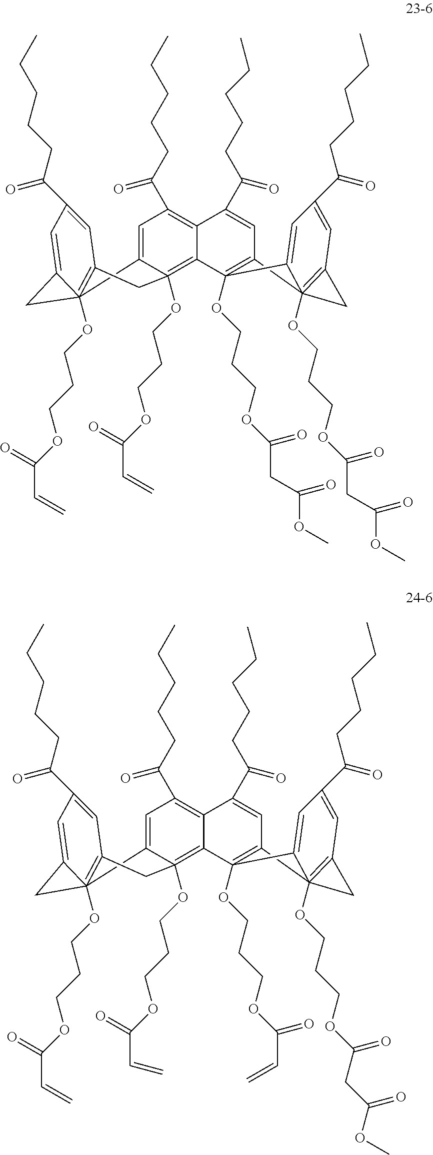

### Example 11

The same procedure as in Example 9 was repeated except that monoethyl malonate was used instead of monomethyl malonate, to thereby obtain 0.445 g of the target compound 25-6 with a yield of 32.0%, 0.201 g of the target compound 26-6 with a yield of 14.9%, 0.208 g of the target compound 27-6 with a yield of 15.4%, and 0.143 g of the target compound 28-6 with a yield of 11.0%.

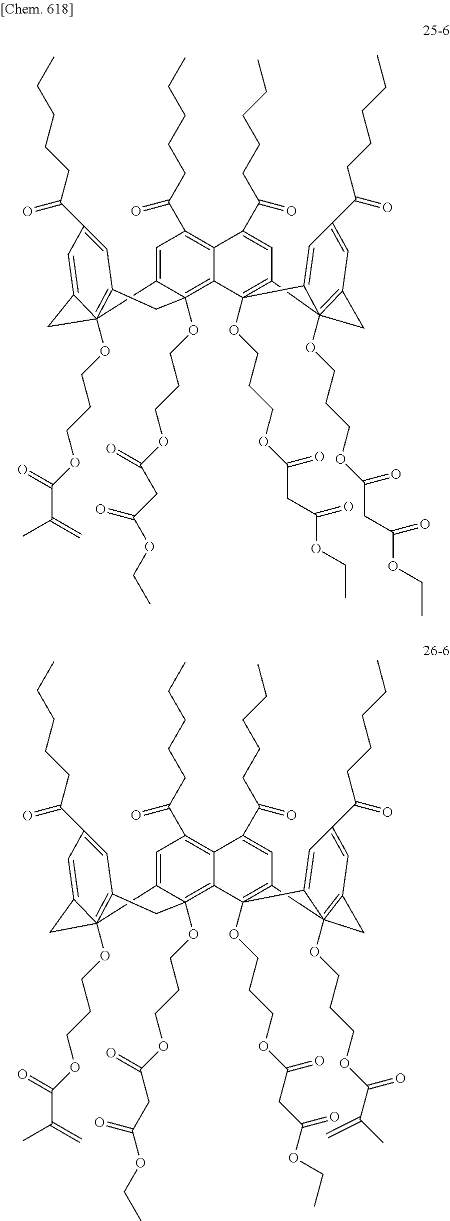

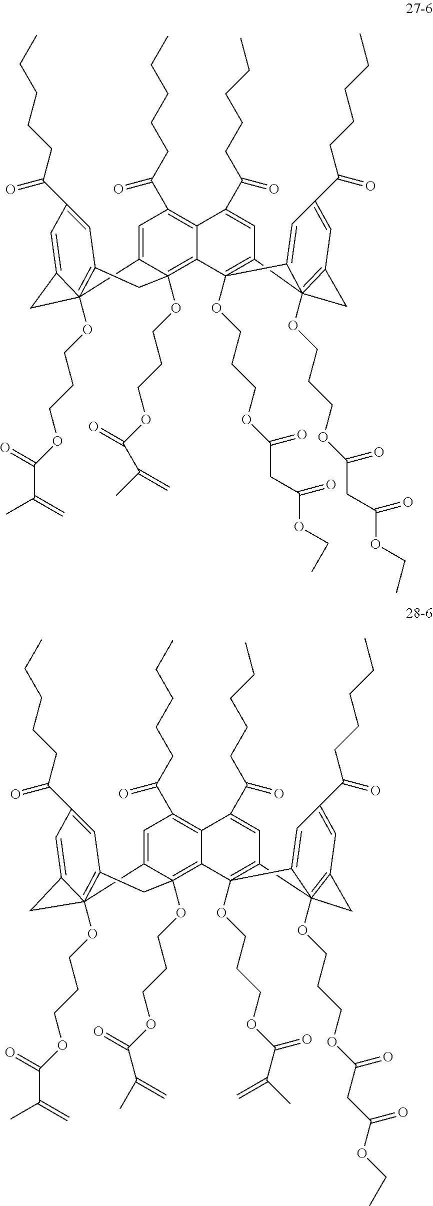

### Example 12

The same procedure as in Example 11 was repeated except that acrylic acid was used instead of methacrylic acid, to thereby obtain 0.401 g of the target compound 29-6 with a yield of 29.1%, 0.198 g of the target compound 30-6 with a yield of 15.0%, 0.187 g of the target compound 31-6 with a yield of 14.2%, and 0.126 g of the target compound 32-6 with a yield of 10.0%.

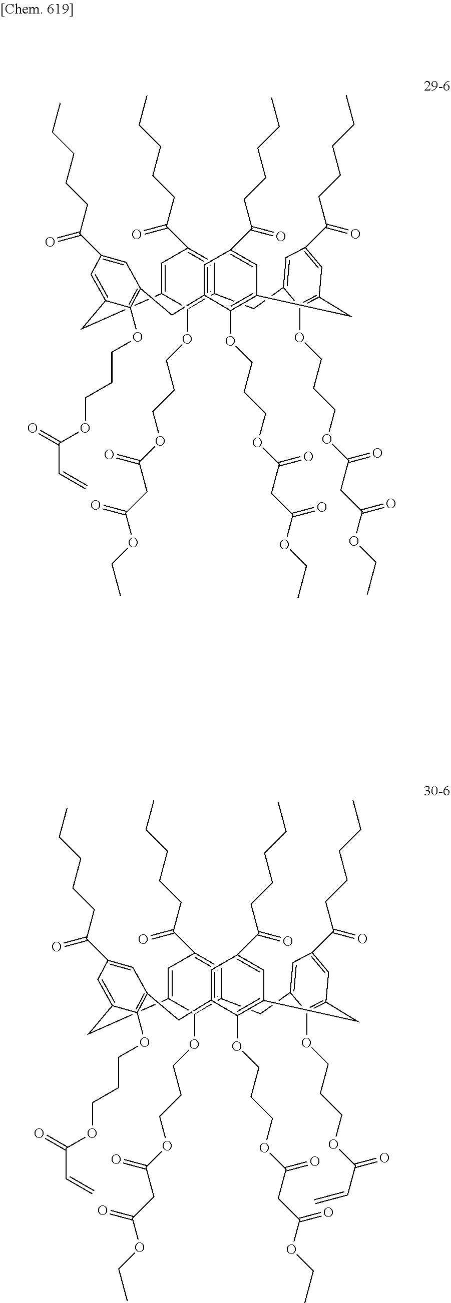 [SMILES: C=CC(=O)OCCCOc1c2cc(C(=O)CCCCC)cc1Cc1cc(C(=O)CCCCC)cc(c1OCCCOC(=O)CC(=O)OCC)Cc1cc(C(=O)CCCCC)cc(c1OCCCOC(=O)CC(=O)OCC)Cc1cc(C(=O)CCCCC)cc(c1OCCCOC(=O)CC(=O)OCC)C2 ; C=CC(=O)OCCCOc1c2cc(C(=O)CCCCC)cc1Cc1cc(C(=O)CCCCC)cc(c1OCCCOC(=O)CC(=O)OCC)Cc1cc(C(=O)CCCCC)cc(c1OCCCOC(=O)C=C)Cc1cc(C(=O)CCCCC)cc(c1OCCCOC(=O)CC(=O)OCC)C2]

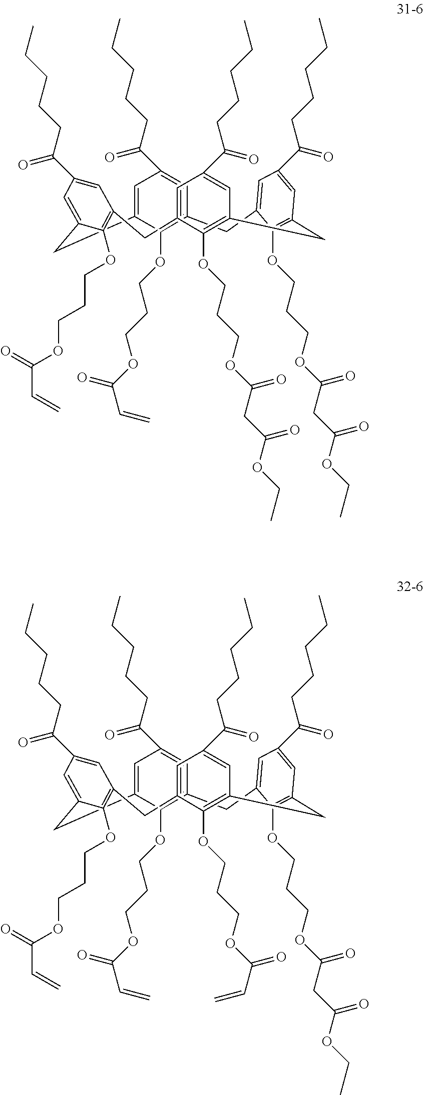 [SMILES: C=CC(=O)OCCCOc1c2cc(C(=O)CCCCC)cc1Cc1cc(C(=O)CCCCC)cc(c1OCCCOC(=O)CC(=O)OCC)Cc1cc(C(=O)CCCCC)cc(c1OCCCOC(=O)CC(=O)OCC)Cc1cc(C(=O)CCCCC)cc(c1OCCCOC(=O)C=C)C2 ; C=CC(=O)OCCCOc1c2cc(C(=O)CCCCC)cc1Cc1cc(C(=O)CCCCC)cc(c1OCCCOC(=O)C=C)Cc1cc(C(=O)CCCCC)cc(c1OCCCOC(=O)CC(=O)OCC)Cc1cc(C(=O)CCCCC)cc(c1OCCCOC(=O)C=C)C2]

### Synthesis Example 15

A 500 mL four-necked flask equipped with a stirrer, a thermometer, and a reflux condenser tube was charged with 92.6 g (113.33 mmol) of B-6 and 944.52 g of diethylene glycol monomethyl ether, and the mixture was stirred. Then 46.4 mL (906.64 mmol) of hydrazine monohydrate and 50.9 g (906.64 mmol) of potassium hydroxide pellets were added, and the resulting mixture was stirred at 100° C. for 30 minutes and further heat-refluxed for 8 hours. After completion of the reaction, the resulting mixture was cooled to 90° C., and 92.6 mL of ion exchanged water was added. The resulting mixture was cooled to room temperature. The solution mixture was transferred to a beaker, and 6N hydrochloric acid was added until the pH was 1. Then 300 g of chloroform was added. The reaction mixture was transferred to a separatory funnel, and the organic layer was separated. Then the aqueous layer was extracted with 300 g of chloroform three times, and the extract was combined with the organic layer. The organic layer was pre-dried over anhydrous magnesium sulfate, and the solvent was removed by evaporation using an evaporator to thereby obtain an orange viscus liquid. Methanol was added to reprecipitate crystals, and the generated white crystals were filtered and vacuum dried (at 60° C. for 6 hours or longer) to thereby obtain 54.34 g of compound G-6 represented by the following structural formula. The yield was 63.0%.

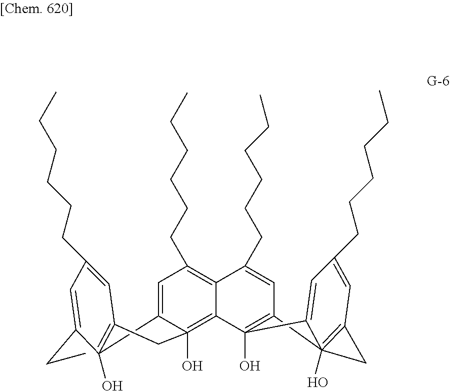

### Synthesis Example 16

The same procedure as in Synthesis Example 15 was repeated except that B-4 was used instead of B-6, to thereby obtain 72.45 g of compound G-4 represented by the following structural formula. The yield was 83.1%.

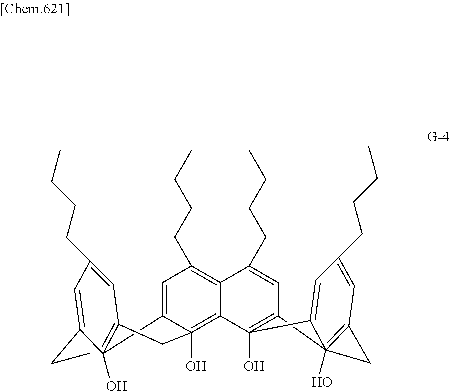

### Synthesis Example 17

The same procedure as in Synthesis Example 15 was repeated except that B-7 was used instead of B-6, to thereby obtain 78.4 g of compound G-7 represented by the following structural formula. The yield was 82.7%.

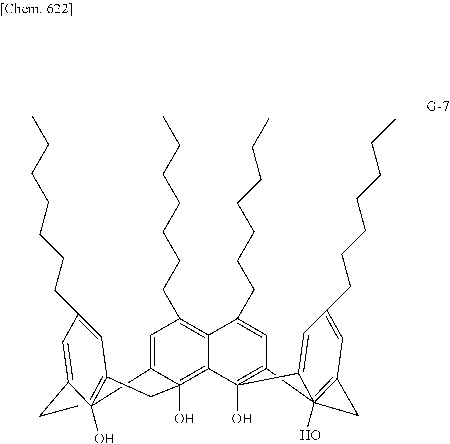

### Synthesis Example 18

The same procedure as in Synthesis Example 15 was repeated except that B-18 was used instead of B-6, to thereby obtain 37.9 g of compound G-18 represented by the following structural formula. The yield was 96.0%.

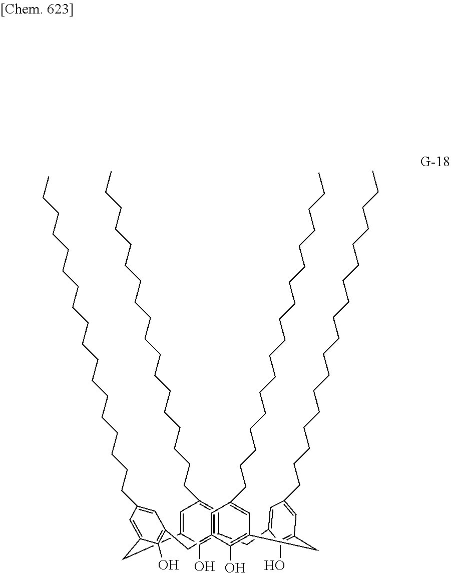 [SMILES: CCCCCCCCCCCCCCCCCCc1cc2c(O)c(c1)Cc1cc(CCCCCCCCCCCCCCCCCC)cc(c1O)Cc1cc(CCCCCCCCCCCCCCCCCC)cc(c1O)Cc1cc(CCCCCCCCCCCCCCCCCC)cc(c1O)C2]

### Synthesis Example 19

Referring to known documents (Tetrahedron Letters, 43(43), 7691-7693; 2002 and Tetrahedron Letters, 48(5), 905-12; 1992), compound G-1 represented by the following structural formula was synthesized according to the following two-step scheme (yield amount 75 g, yield 66.6%).

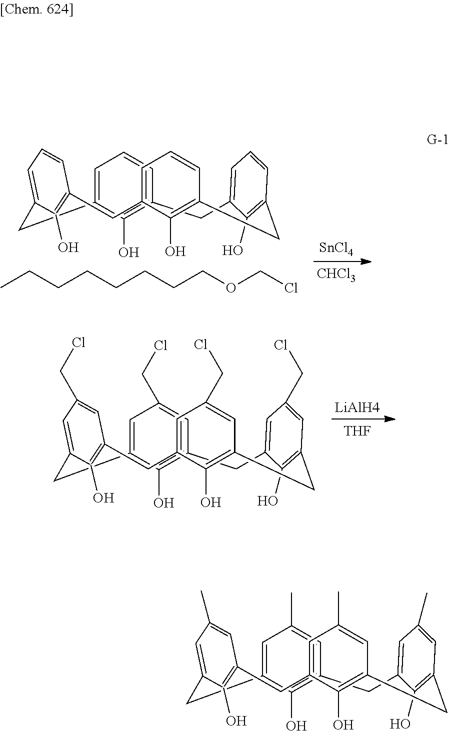

### Synthesis Example 20

A 1 L four-necked flask equipped with a stirrer, a thermometer, and a reflux condenser tube was charged with 20.00 g (26.276 mmol) of G-6, 400 g of acetonitrile, 15.29 g (105.11 mmol) of potassium carbonate, 10.511 g (10.511 mmol) of potassium iodide, and 32.158 g (210.21 mmol) of methyl 2-bromoacetate, and the mixture was allowed to react at 70° C. for 6 hours. The resulting mixture was cooled to room temperature, and then ion exchanged water and 1N hydrochloric acid were added until the pH was 6. After 500 g of chloroform was added, the reaction mixture was transferred to a separatory funnel, and the organic layer was separated. Then the aqueous layer was extracted with 100 g of chloroform three times, and the extract was combined with the organic layer. The organic layer was pre-dried over anhydrous magnesium sulfate, and the solvent was removed by evaporation using an evaporator to thereby obtain a red waxy solid. The obtained red waxy solid was vacuum dried (at 60° C. for 6 hours or longer) to thereby obtain 21.67 g of compound H-6 represented by the following structural formula. The yield was 78.6%.

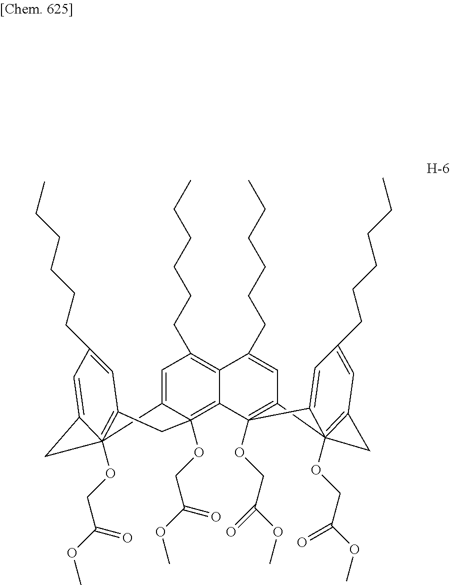

### Synthesis Example 21

The same procedure as in Synthesis Example 20 was repeated except that G-4 was used instead of G-6, to thereby obtain 21.81 g of compound H-4 represented by the following structural formula. The yield was 75.5%.

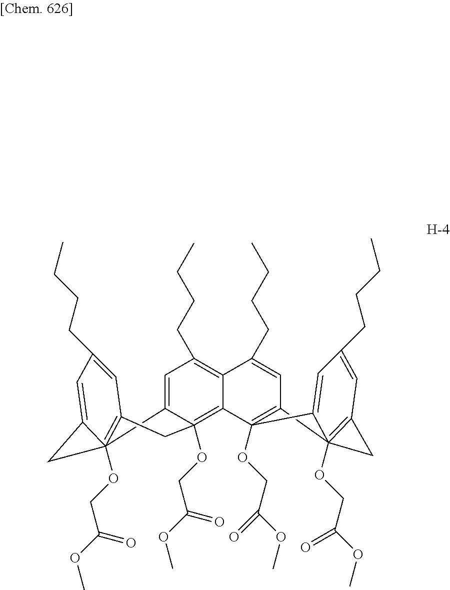

### Synthesis Example 22

The same procedure as in Synthesis Example 20 was repeated except that G-7 was used instead of G-6, to thereby obtain 20.98 g of compound H-7 represented by the following structural formula. The yield was 77.5%.

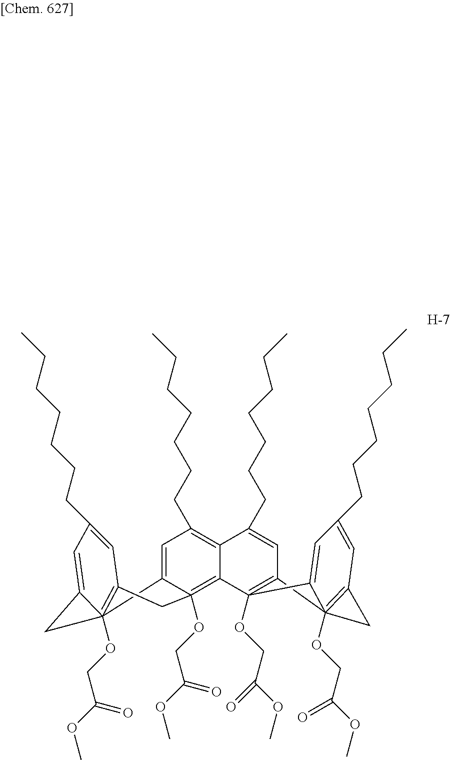

### Synthesis Example 23

The same procedure as in Synthesis Example 20 was repeated except that G-18 was used instead of G-6, to thereby obtain 19.32 g of compound H-18 represented by the following structural formula. The yield was 80.4%.

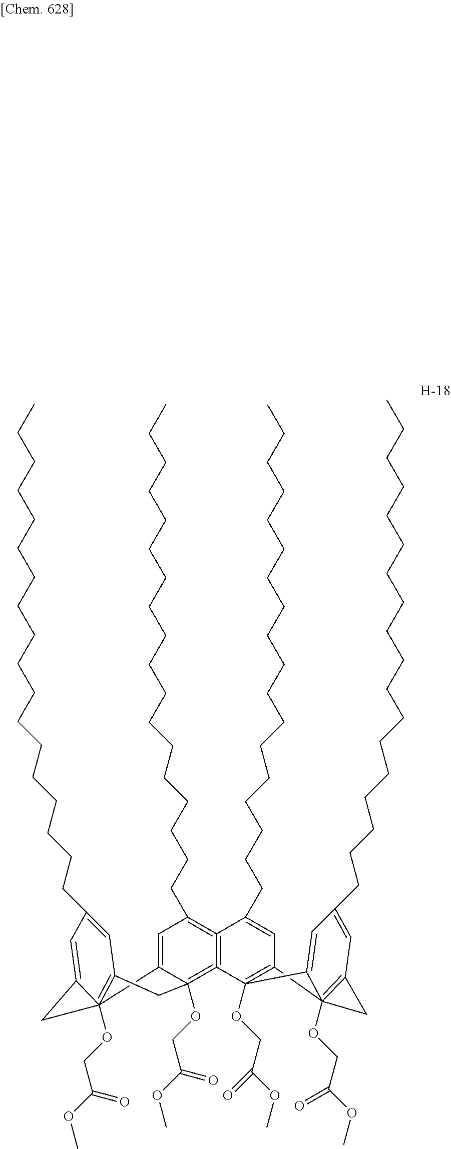

### Synthesis Example 24

The same procedure as in Synthesis Example 20 was repeated except that G-1 was used instead of G-6, to thereby obtain 18.32 g of compound H-1 represented by the following structural formula. The yield was 57.3%.

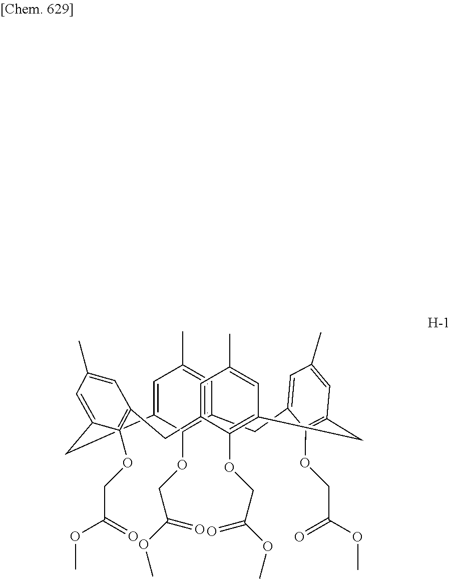 [SMILES: COC(=O)COc1c2cc(C)cc1Cc1cc(C)cc(c1OCC(=O)OC)Cc1cc(C)cc(c1OCC(=O)OC)Cc1cc(C)cc(c1OCC(=O)OC)C2]

### Synthesis Example 25

The same procedure as in Synthesis Example 10 was repeated except that H-6 was used instead of C-6, to thereby obtain 6.12 g of compound I-6 represented by the following structural formula. The yield was 68.5%.

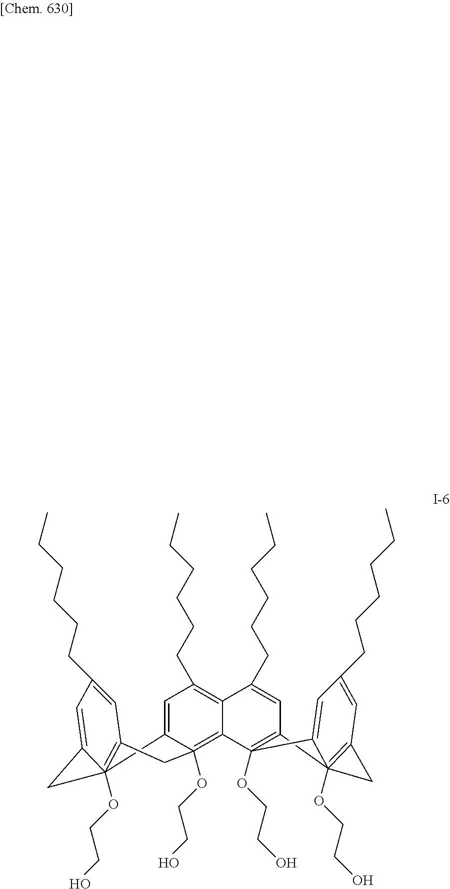

### Synthesis Example 26

The same procedure as in Synthesis Example 25 was repeated except that H-4 was used instead of H-6, to thereby obtain 4.21 g of compound I-4 represented by the following structural formula. The yield was 81.4%.

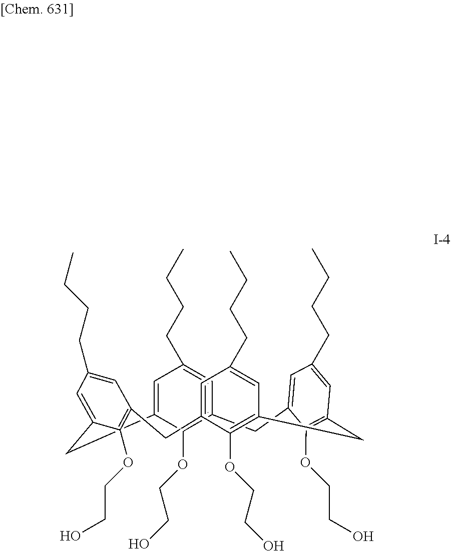 [SMILES: CCCCc1cc2c(OCCO)c(c1)Cc1cc(CCCC)cc(c1OCCO)Cc1cc(CCCC)cc(c1OCCO)Cc1cc(CCCC)cc(c1OCCO)C2]

### Synthesis Example 27

The same procedure as in Synthesis Example 25 was repeated except that H-7 was used instead of H-6, to thereby obtain 3.89 g of compound I-7 represented by the following structural formula. The yield was 84.5%.

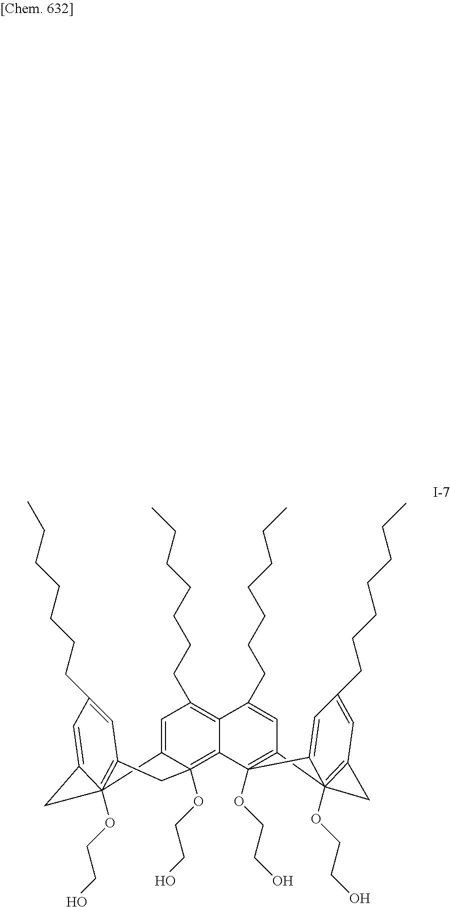

### Synthesis Example 28

The same procedure as in Synthesis Example 25 was repeated except that H-18 was used instead of H-6, to thereby obtain 4.31 g of compound I-18 represented by the following structural formula. The yield was 81.7%.

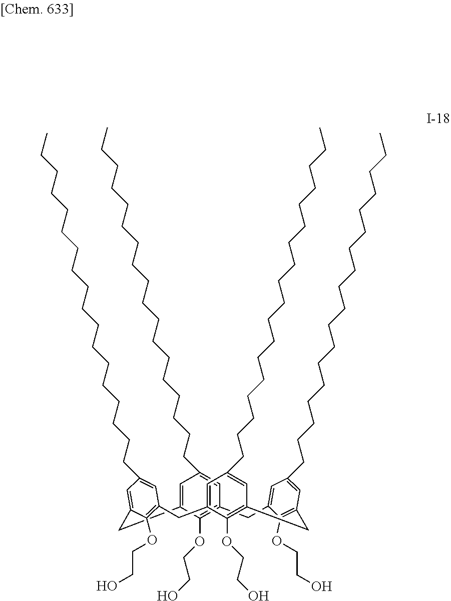 [SMILES: CCCCCCCCCCCCCCCCCCc1cc2c(OCCO)c(c1)Cc1cc(CCCCCCCCCCCCCCCCCC)cc(c1OCCO)Cc1cc(CCCCCCCCCCCCCCCCCC)cc(c1OCCO)Cc1cc(CCCCCCCCCCCCCCCCCC)cc(c1OCCO)C2]

### Synthesis Example 29

The same procedure as in Synthesis Example 25 was repeated except that H-1 was used instead of H-6, to thereby obtain 3.43 g of compound I-1 represented by the following structural formula. The yield was 85.1%.

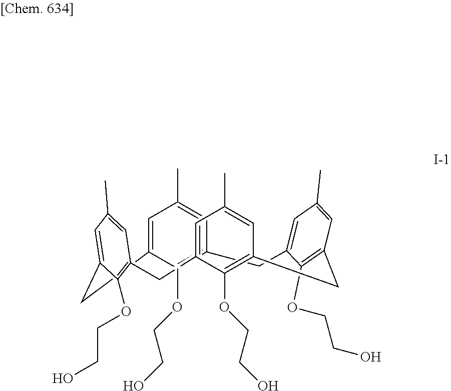 [SMILES: Cc1cc2c(OCCO)c(c1)Cc1cc(C)cc(c1OCCO)Cc1cc(C)cc(c1OCCO)Cc1cc(C)cc(c1OCCO)C2]

### Example 13

The same procedure as in Example 1 was repeated except that I-6 was used instead of D-6, to thereby obtain 0.511 g of the target compound 33-6 with a yield of 36.7%, 0.276 g of the target compound 34-6 with a yield of 20.3%, 0.221 g of the target compound 35-6 with a yield of 16.3%, and 0.114 g of the target compound 36-6 with a yield of 8.61%.

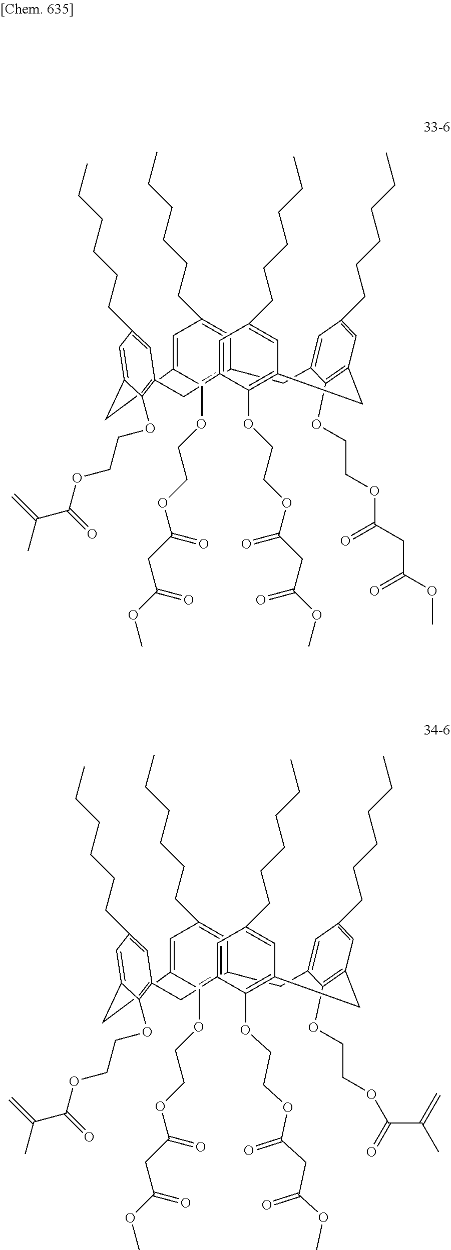

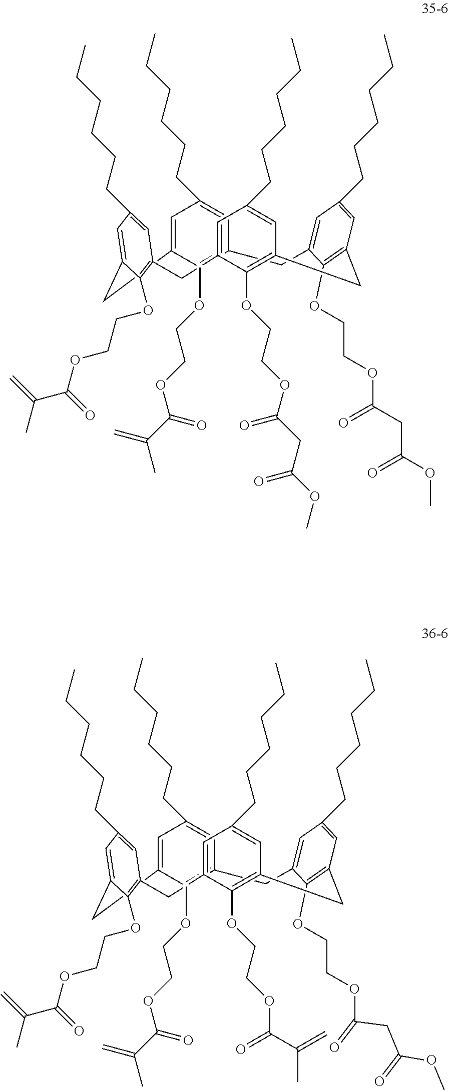

### Example 14

The same procedure as in Example 13 was repeated except that I-4 was used instead of I-6, to thereby obtain 0.506 g of the target compound 33-4 with a yield of 35.0%, 0.245 g of the target compound 34-4 with a yield of 17.4%, 0.221 g of the target compound 35-4 with a yield of 15.7%, and 0.141 g of the target compound 36-4 with a yield of 10.3%.

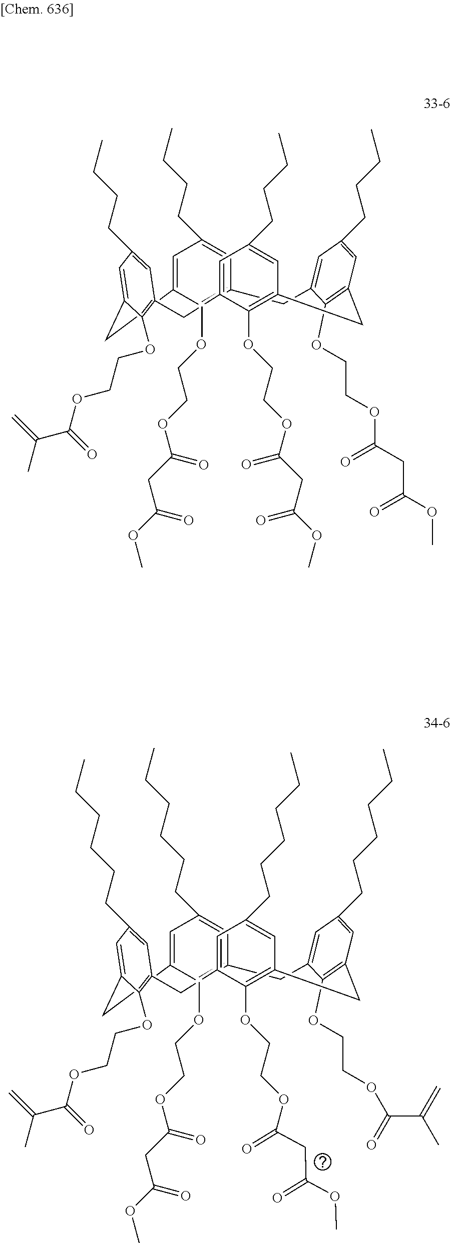

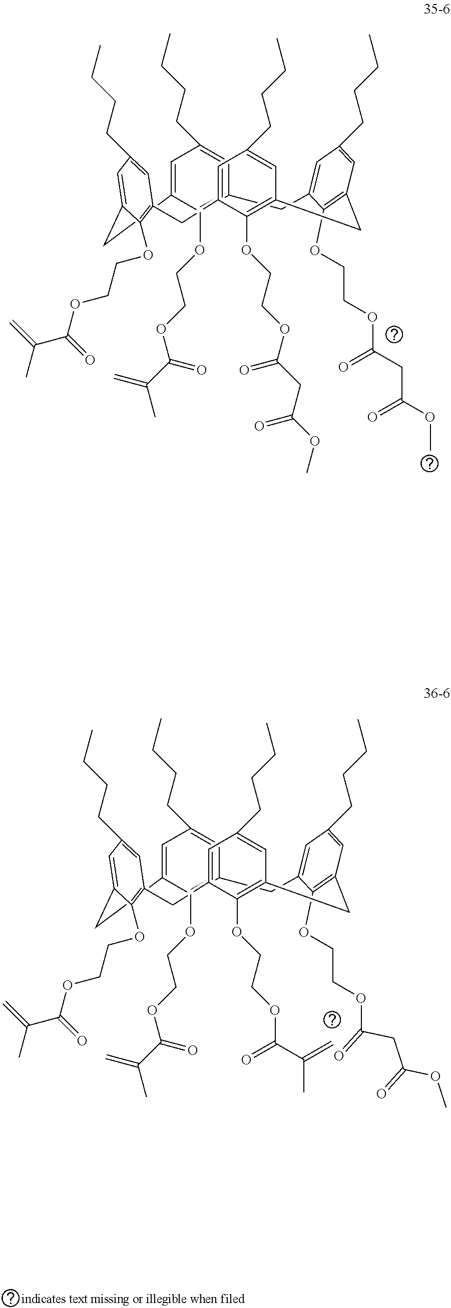

### Example 15

The same procedure as in Example 13 was repeated except that I-7 was used instead of I-6, to thereby obtain 0.528 g of the target compound 33-7 with a yield of 38.5%, 0.234 g of the target compound 34-7 with a yield of 17.5%, 0.237 g of the target compound 35-7 with a yield of 17.7%, and 0.129 g of the target compound 36-7 with a yield of 9.88%.

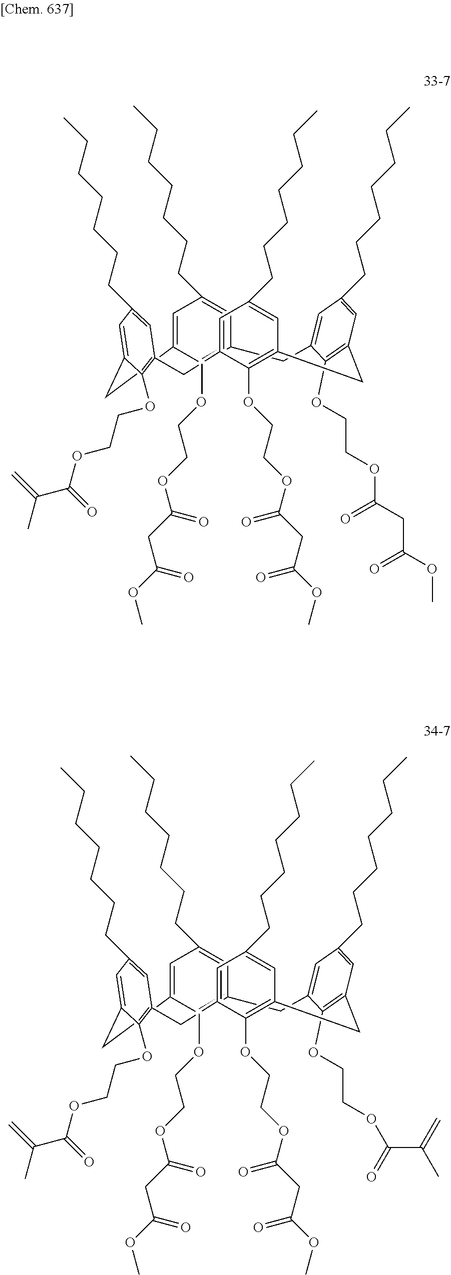

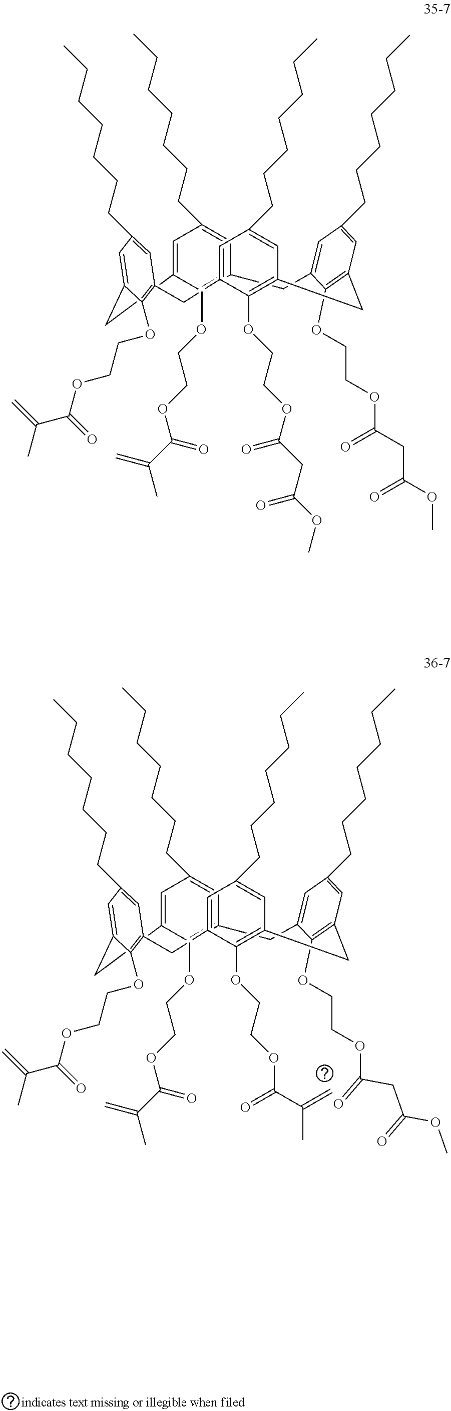

### Example 16

The same procedure as in Example 13 was repeated except that I-18 was used instead of I-6, to thereby obtain 0.513 g of the target compound 33-18 with a yield of 41.8%, 0.213 g of the target compound 34-18 with a yield of 17.6%, 0.211 g of the target compound 35-18 with a yield of 17.5%, and 0.102 g of the target compound 36-18 with a yield of 8.58%.

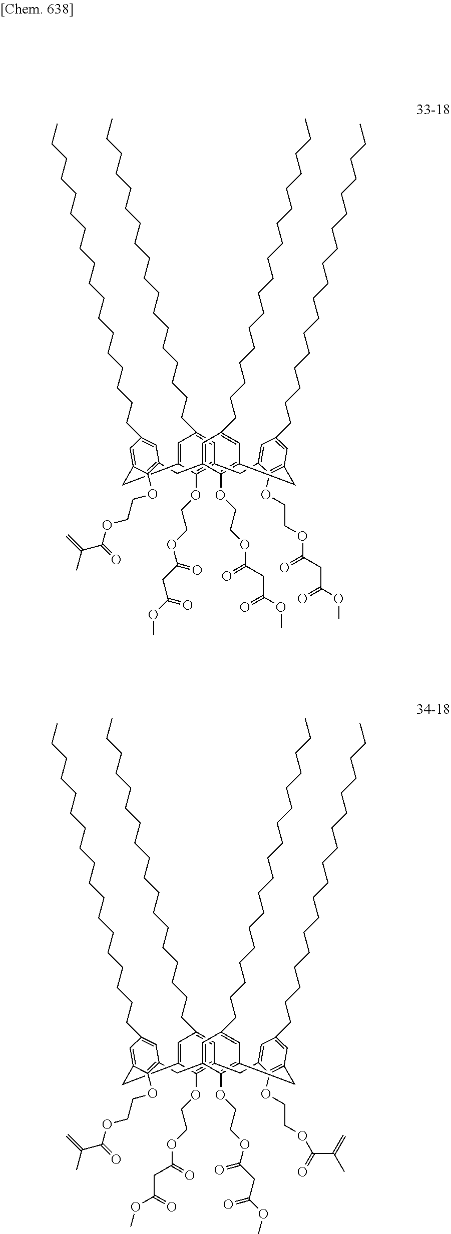 [SMILES: C=C(C)C(=O)OCCOc1c2cc(CCCCCCCCCCCCCCCCCC)cc1Cc1cc(CCCCCCCCCCCCCCCCCC)cc(c1OCCOC(=O)CC(=O)OC)Cc1cc(CCCCCCCCCCCCCCCCCC)cc(c1OCCOC(=O)CC(=O)OC)Cc1cc(CCCCCCCCCCCCCCCCCC)cc(c1OCCOC(=O)CC(=O)OC)C2 ; C=C(C)C(=O)OCCOc1c2cc(CCCCCCCCCCCCCCCCCC)cc1Cc1cc(CCCCCCCCCCCCCCCCCC)cc(c1OCCOC(=O)CC(=O)OC)Cc1cc(CCCCCCCCCCCCCCCCCC)cc(c1OCCOC(=O)C(=C)C)Cc1cc(CCCCCCCCCCCCCCCCCC)cc(c1OCCOC(=O)CC(=O)OC)C2]

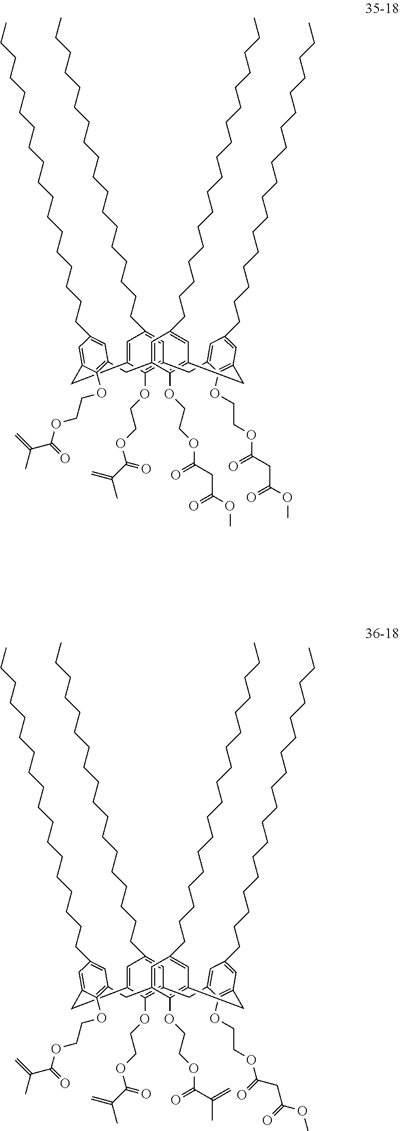 [SMILES: C=C(C)C(=O)OCCOc1c2cc(CCCCCCCCCCCCCCCCCC)cc1Cc1cc(CCCCCCCCCCCCCCCCCC)cc(c1OCCOC(=O)C(=C)C)Cc1cc(CCCCCCCCCCCCCCCCCC)cc(c1OCCOC(=O)CC(=O)OC)Cc1cc(CCCCCCCCCCCCCCCCCC)cc(c1OCCOC(=O)CC(=O)OC)C2 ; C=C(C)C(=O)OCCOc1c2cc(CCCCCCCCCCCCCCCCCC)cc1Cc1cc(CCCCCCCCCCCCCCCCCC)cc(c1OCCOC(=O)C(=C)C)Cc1cc(CCCCCCCCCCCCCCCCCC)cc(c1OCCOC(=O)C(=C)C)Cc1cc(CCCCCCCCCCCCCCCCCC)cc(c1OCCOC(=O)CC(=O)OC)C2]

### Example 17

The same procedure as in Example 13 was repeated except that I-1 was used instead of I-6, to thereby obtain 0.487 g of the target compound 33-1 with a yield of 31.27%, 0.217 g of the target compound 34-1 with a yield of 14.4%, 0.221 g of the target compound 35-1 with a yield of 14.6%, and 0.178 g of the target compound 36-1 with a yield of 12.2%.

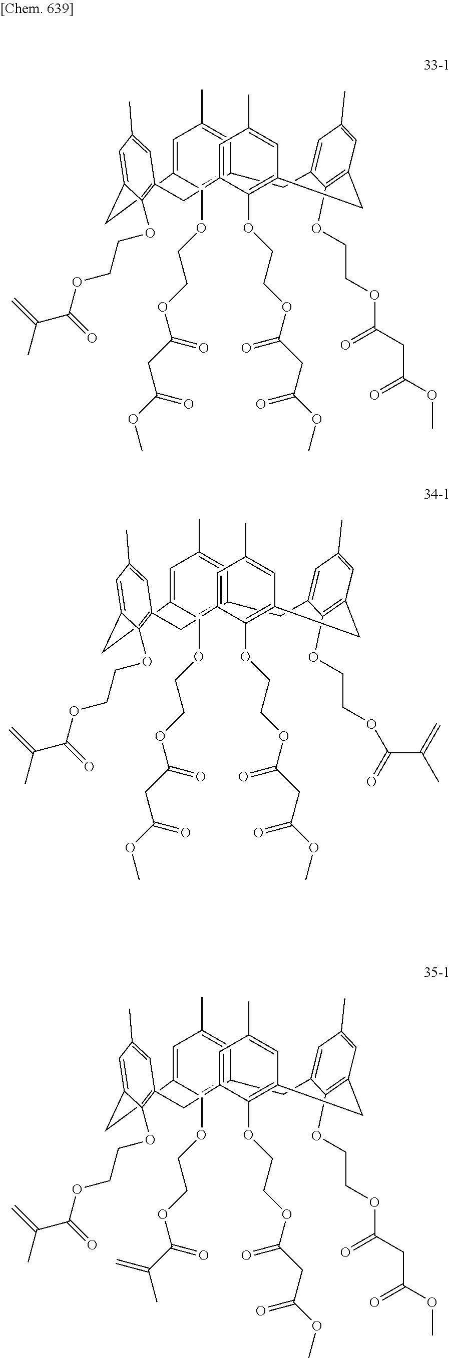

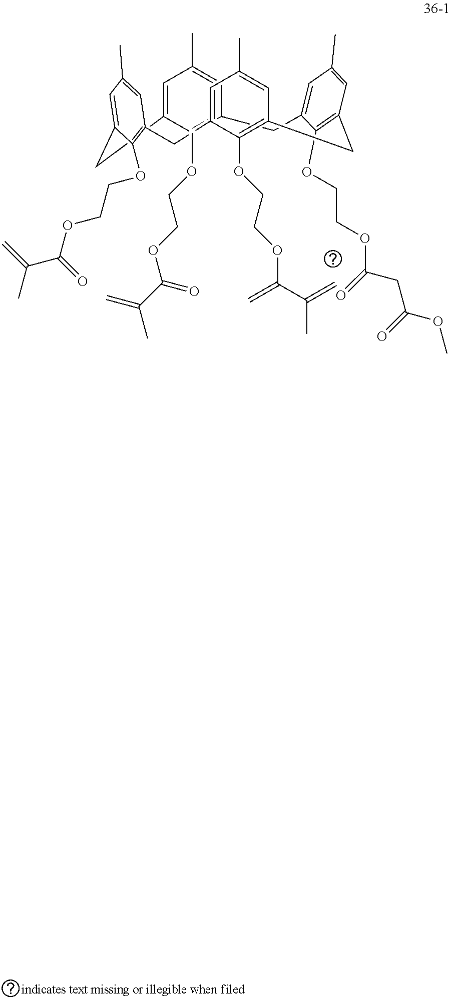 [SMILES: C=C(C)C(=C)OCCOc1c2cc(C)cc1Cc1cc(C)cc(c1OCCOC(=O)C(=C)C)Cc1cc(C)cc(c1OCCOC(=O)C(=C)C)Cc1cc(C)cc(c1OCCOC(=O)CC(=O)OC)C2]

### Example 18

The same procedure as in Example 13 was repeated except that acrylic acid was used instead of methacrylic acid, to thereby obtain 0.462 of the target compound 37-6 with a yield of 33.5%, 0.208 g of the target compound 38-6 with a yield of 15.7%, 0.198 g of the target compound 39-6 with a yield of 14.9%, and 0.135 g of the target compound 40-6 with a yield of 10.5%.

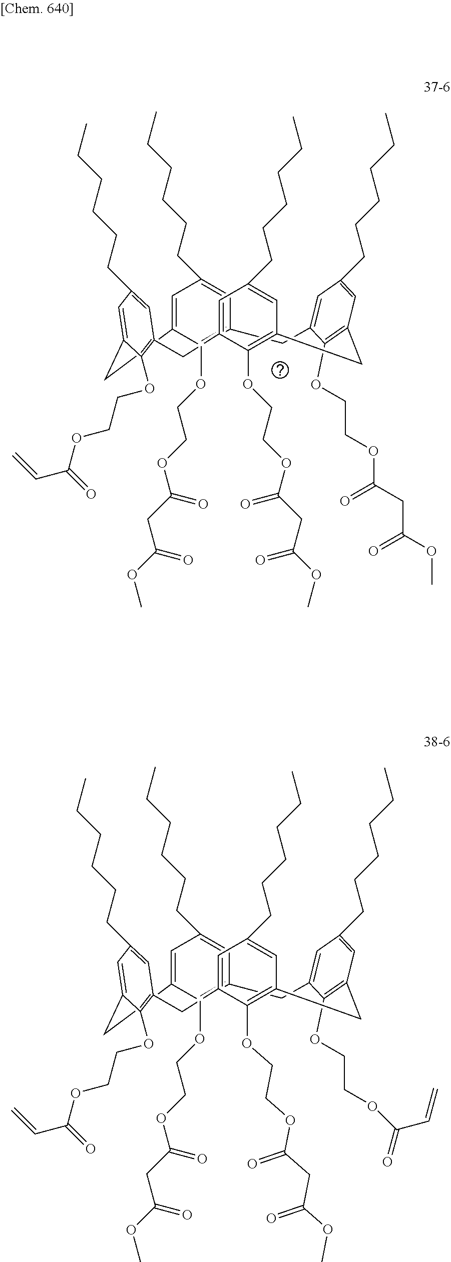

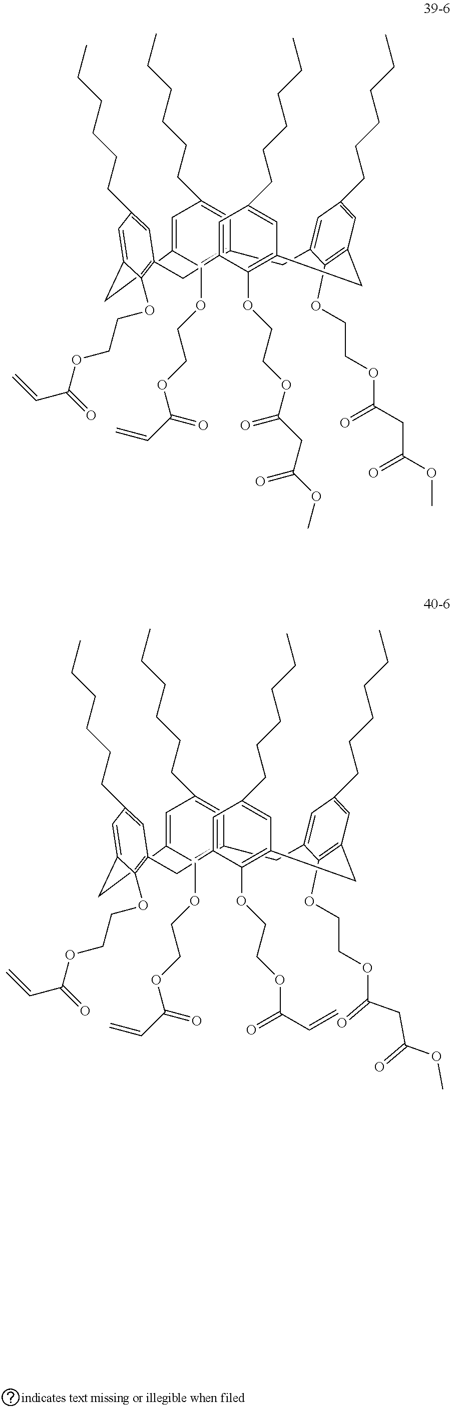

### Example 19

The same procedure as in Example 13 was repeated except that monoethyl malonate was used instead of monomethyl malonate, to thereby obtain 0.451 g of the target compound 41-6 with a yield of 31.4%, 0.228 g of the target compound 42-6 with a yield of 17.8%, 0.219 g of the target compound 43-6 with a yield of 15.8%, and 0.218 g of the target compound 44-6 with a yield of 16.3%.

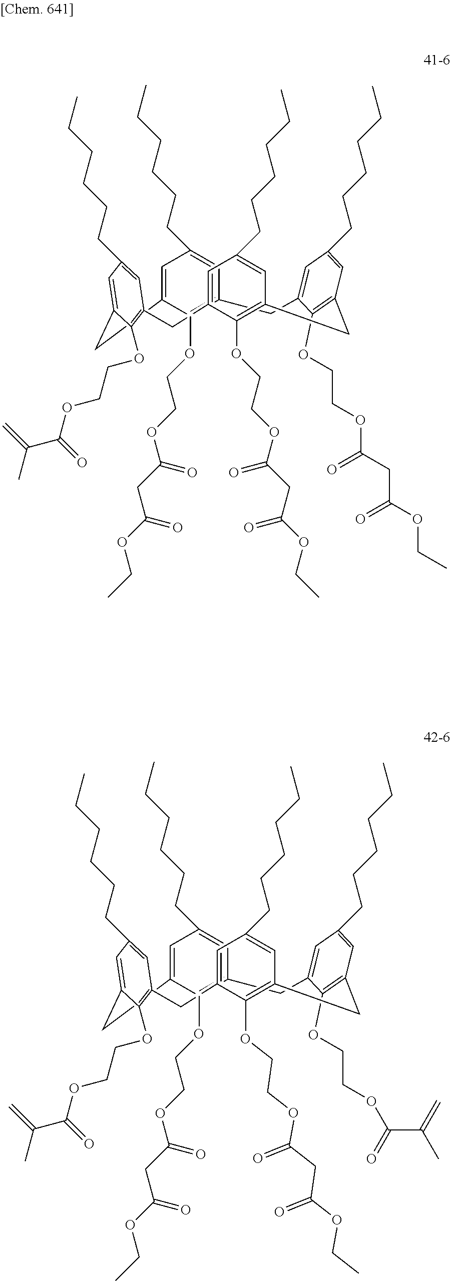

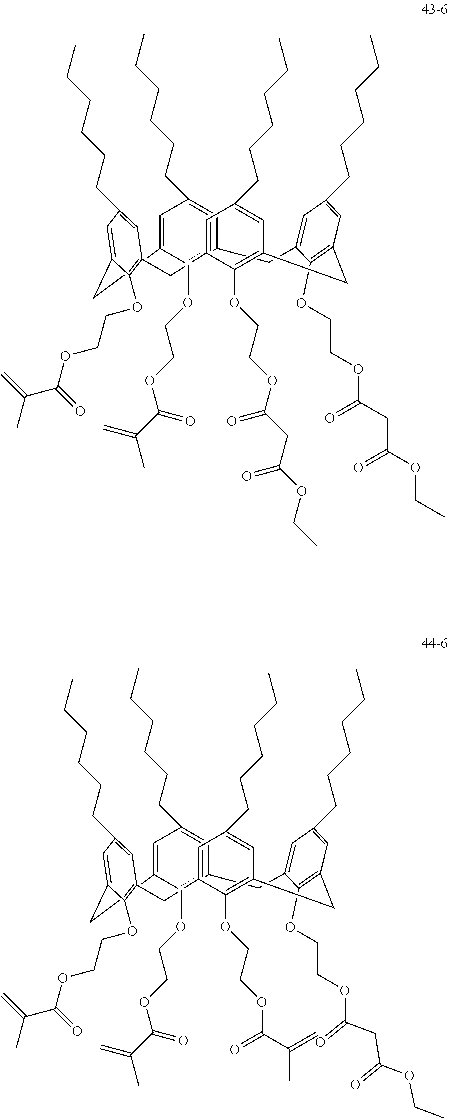

### Example 20

The same procedure as in Example 19 was repeated except that monoethyl malonate was used instead of monomethyl malonate, to thereby obtain 0.402 g of the target compound 45-6 with a yield of 28.3%, 0.218 g of the target compound 46-6 with a yield of 16.0%, 0.221 g of the target compound 47-6 with a yield of 16.3%, and 0.172 g of the target compound 48-6 with a yield of 13.3%.

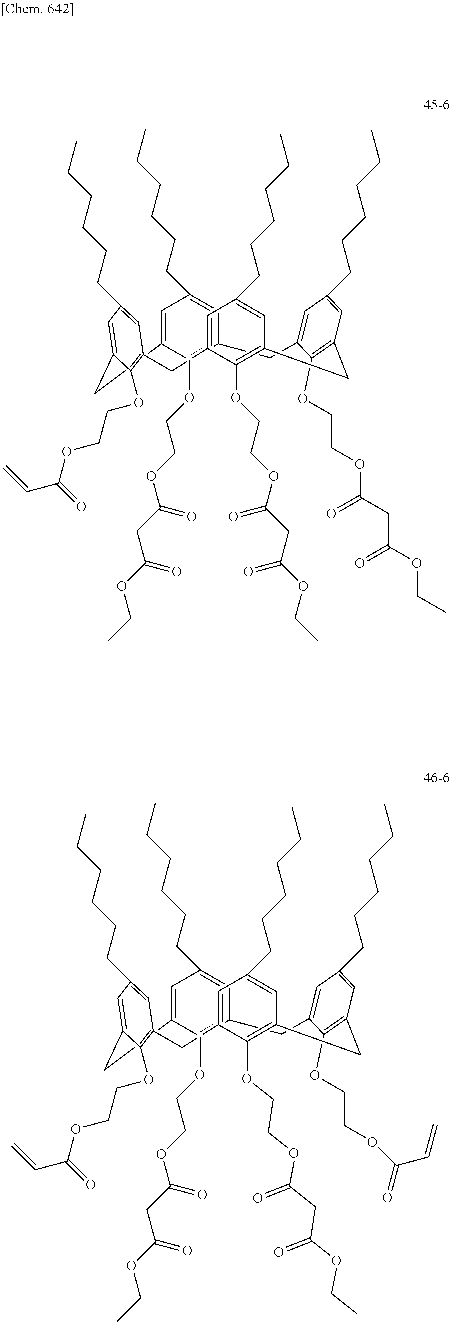

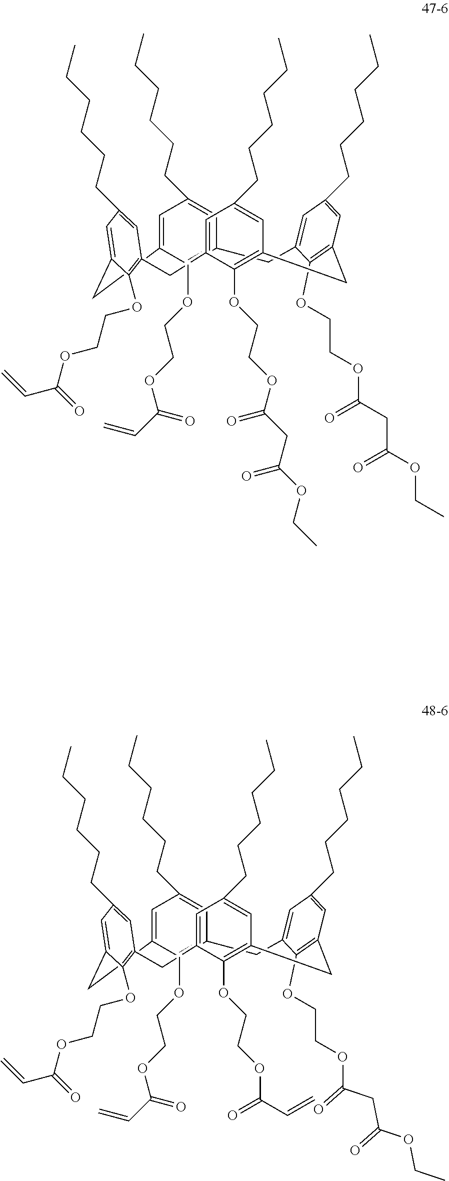

### Synthesis Example 30

The same procedure as in Synthesis Example 20 was repeated except that methyl bromopyopionate was used instead of methyl bromoacetate, to thereby obtain 4.89 g of compound J-6 represented by the following structural formula. The yield was 67.3%.

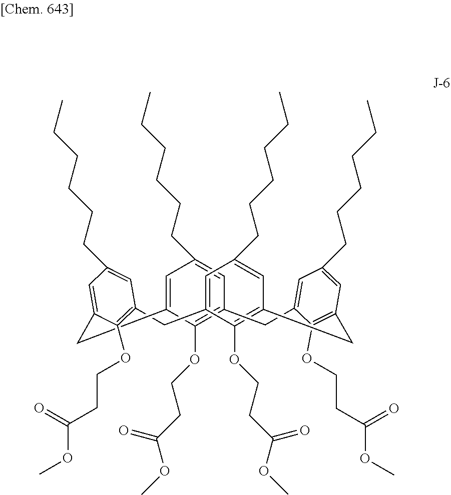 [SMILES: CCCCCCc1cc2c(OCCC(=O)OC)c(c1)Cc1cc(CCCCCC)cc(c1OCCC(=O)OC)Cc1cc(CCCCCC)cc(c1OCCC(=O)OC)Cc1cc(CCCCCC)cc(c1OCCC(=O)OC)C2]

### Synthesis Example 31

The same procedure as in Synthesis Example 10 was repeated except that J-6 was used instead of C-6, to thereby obtain 3.88 g of compound K-6 represented by the following structural formula. The yield was 88.3%.

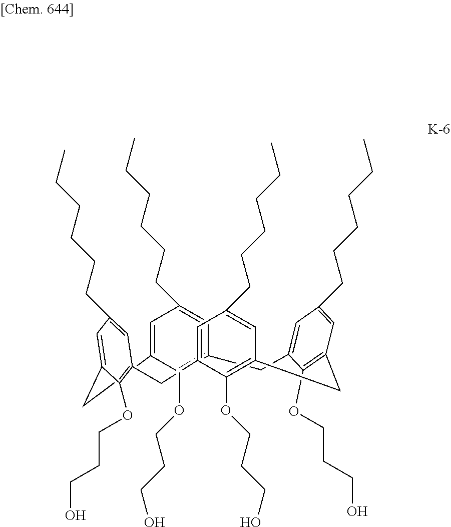 [SMILES: CCCCCCc1cc2c(OCCCO)c(c1)Cc1cc(CCCCCC)cc(c1OCCCO)Cc1cc(CCCCCC)cc(c1OCCCO)Cc1cc(CCCCCC)cc(c1OCCCO)C2]

### Example 21

The same procedure as in Example 1 was repeated except that K-6 was used instead of D-6, to thereby obtain 0.366 g of the target compound 49-6 with a yield of 26.7%, 0.207 g of the target compound 50-6 with a yield of 15.5%, 0.212 g of the target compound 51-6 with a yield of 15.8%, and 0.198 g of the target compound 52-6 with a yield of 15.2%.

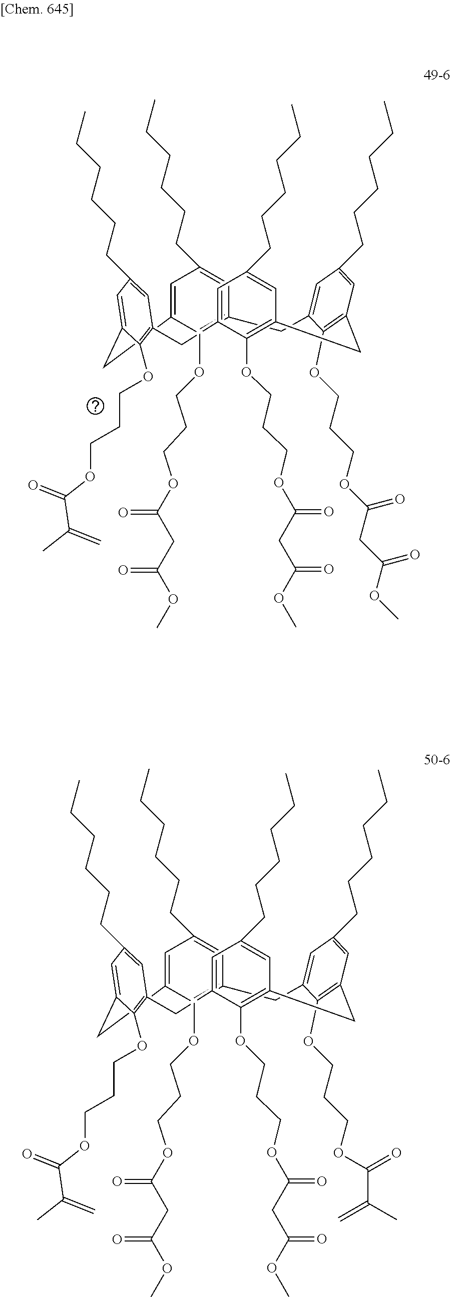 [SMILES: C=C(C)C(=O)OCCCOc1c2cc(CCCCCC)cc1Cc1cc(CCCCCC)cc(c1OCCCOC(=O)CC(=O)OC)Cc1cc(CCCCCC)cc(c1OCCCOC(=O)CC(=O)OC)Cc1cc(CCCCCC)cc(c1OCCCOC(=O)CC(=O)OC)C2 ; C=C(C)C(=O)OCCCOc1c2cc(CCCCCC)cc1Cc1cc(CCCCCC)cc(c1OCCCOC(=O)CC(=O)OC)Cc1cc(CCCCCC)cc(c1OCCCOC(=O)C(=C)C)Cc1cc(CCCCCC)cc(c1OCCCOC(=O)CC(=O)OC)C2]

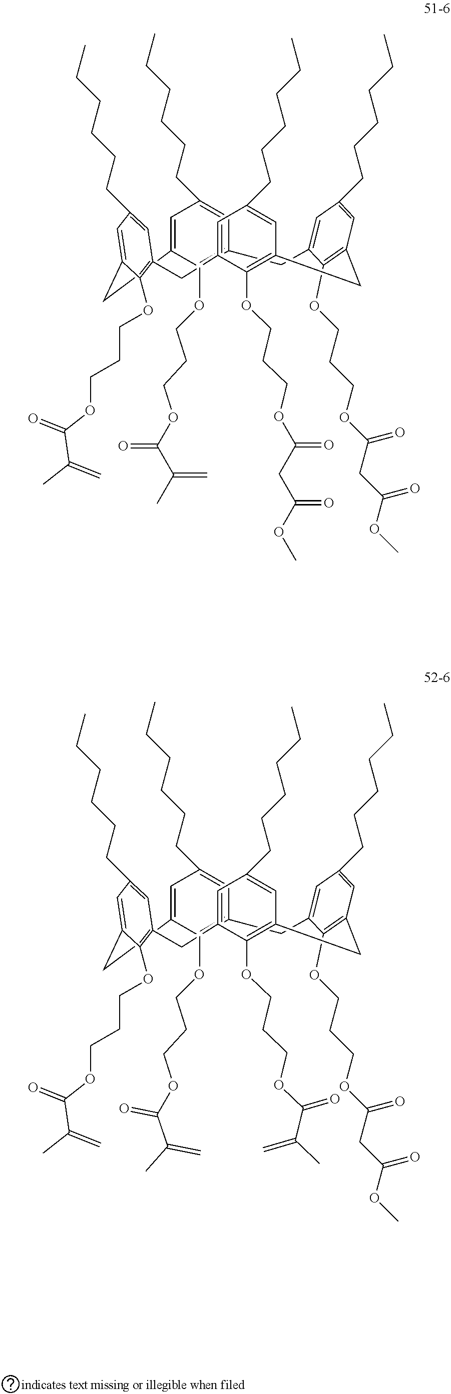 [SMILES: C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)CC(=O)OC)Cc1cc(CCCCCC)cc(c1OCCCOC(=O)CC(=O)OC)C2 ; C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)C(=C)C)Cc1cc(CCCCCC)cc(c1OCCCOC(=O)CC(=O)OC)C2]

### Example 22

The same procedure as in Example 21 was repeated except that acrylic acid was used instead of methacrylic acid, to thereby obtain 0.371 g of the target compound 53-6 with a yield of 27.3%, 0.228 g of the target compound 54-6 with a yield of 17.4%, 0.214 g of the target compound 55-6 with a yield of 16.3%, and 0.174 g of the target compound 56-6 with a yield of 13.8%.

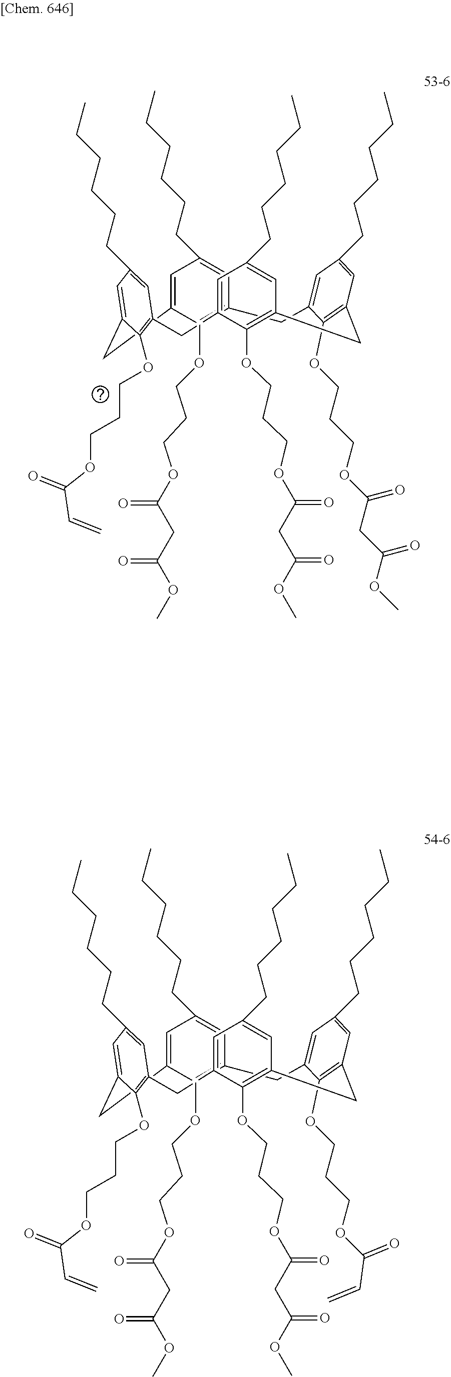 [SMILES: C=CC(=O)OCCCOc1c2cc(CCCCCC)cc1Cc1cc(CCCCCC)cc(c1OCCCOC(=O)CC(=O)OC)Cc1cc(CCCCCC)cc(c1OCCCOC(=O)CC(=O)OC)Cc1cc(CCCCCC)cc(c1OCCCOC(=O)CC(=O)OC)C2 ; C=CC(=O)OCCCOc1c2cc(CCCCCC)cc1Cc1cc(CCCCCC)cc(c1OCCCOC(=O)CC(=O)OC)Cc1cc(CCCCCC)cc(c1OCCCOC(=O)C=C)Cc1cc(CCCCCC)cc(c1OCCCOC(=O)CC(=O)OC)C2]

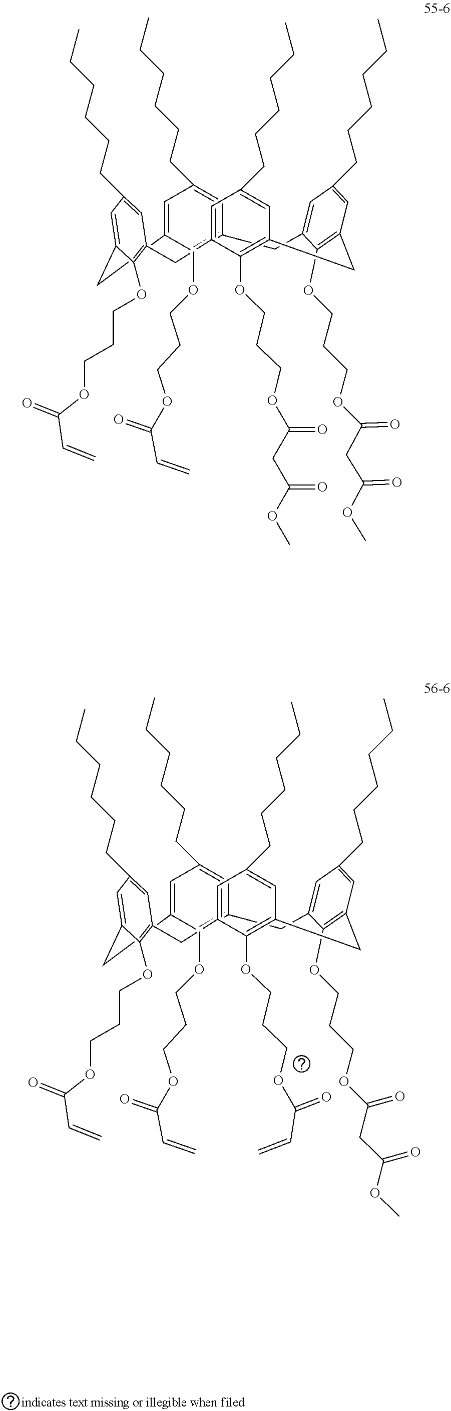 [SMILES: C=CC(=O)OCCCOc1c2cc(CCCCCC)cc1Cc1cc(CCCCCC)cc(c1OCCCOC(=O)CC(=O)OC)Cc1cc(CCCCCC)cc(c1OCCCOC(=O)CC(=O)OC)Cc1cc(CCCCCC)cc(c1OCCCOC(=O)C=C)C2 ; C=CC(=O)OCCCOc1c2cc(CCCCCC)cc1Cc1cc(CCCCCC)cc(c1OCCCOC(=O)C=C)Cc1cc(CCCCCC)cc(c1OCCCOC(=O)CC(=O)OC)Cc1cc(CCCCCC)cc(c1OCCCOC(=O)C=C)C2]

### Example 23

The same procedure as in Example 21 was repeated except that monoethyl malonate was used instead of monomethyl malonate, to thereby obtain 0.402 g of the target compound 57-6 with a yield of 28.4%, 0.234 g of the target compound 58-6 with a yield of 17.1%, 0.209 g of the target compound 59-6 with a yield of 15.3%, and 0.187 g of the target compound 60-6 with a yield of 14.2%.

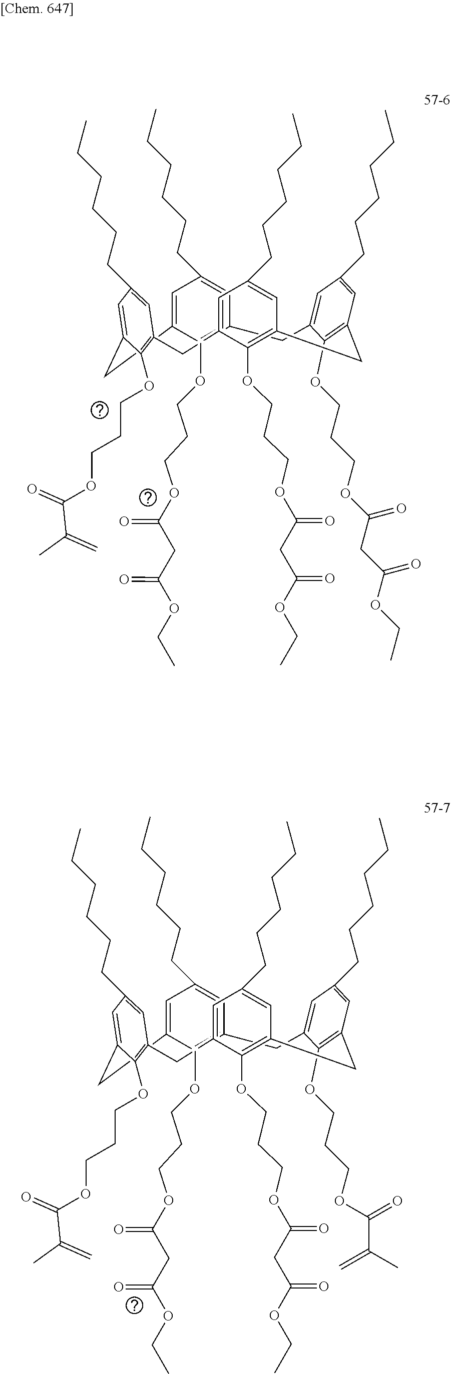 [SMILES: C=C(C)C(=O)OCCCOc1c2cc(CCCCCC)cc1Cc1cc(CCCCCC)cc(c1OCCCOC(=O)CC(=O)OCC)Cc1cc(CCCCCC)cc(c1OCCCOC(=O)CC(=O)OCC)Cc1cc(CCCCCC)cc(c1OCCCOC(=O)CC(=O)OCC)C2 ; C=C(C)C(=O)OCCCOc1c2cc(CCCCCC)cc1Cc1cc(CCCCCC)cc(c1OCCCOC(=O)CC(=O)OCC)Cc1cc(CCCCCC)cc(c1OCCCOC(=O)C(=C)C)Cc1cc(CCCCCC)cc(c1OCCCOC(=O)CC(=O)OCC)C2]

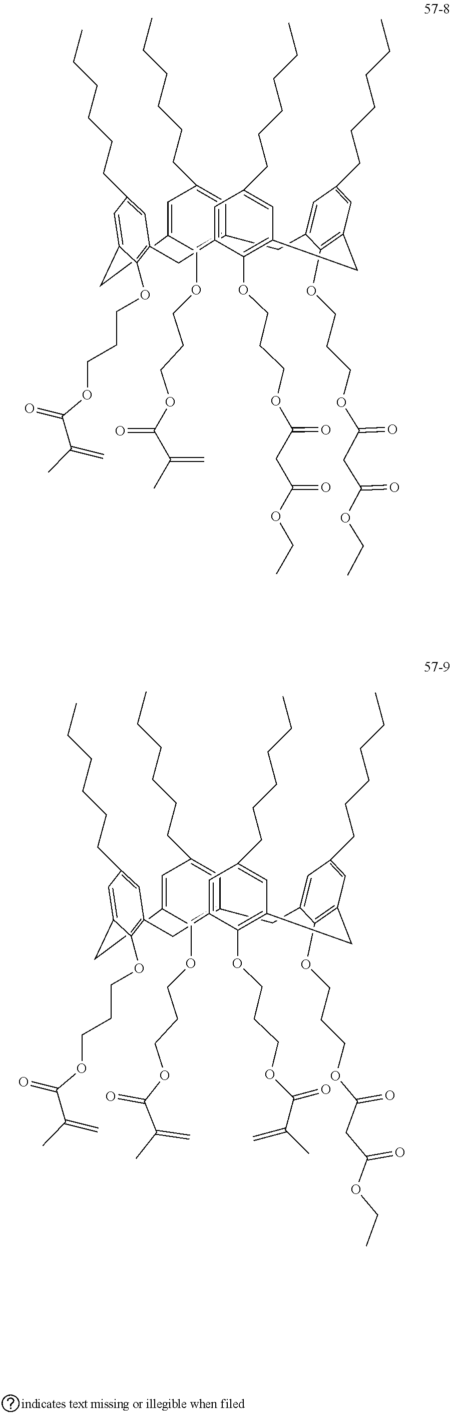 [SMILES: C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)CC(=O)OCC)Cc1cc(CCCCCC)cc(c1OCCCOC(=O)CC(=O)OCC)C2 ; C=C(C)C(=O)OCCCOc1c2cc(CCCCCC)cc1Cc1cc(CCCCCC)cc(c1OCCCOC(=O)C(=C)C)Cc1cc(CCCCCC)cc(c1OCCCOC(=O)C(=C)C)Cc1cc(CCCCCC)cc(c1OCCCOC(=O)CC(=O)OCC)C2]

### Example 24

The same procedure as in Example 23 was repeated except that acrylic acid was used instead of methacrylic acid, to thereby obtain 0.361 g of the target compound 61-6 with a yield of 25.8%, 0.279 g of the target compound 62-6 with a yield of 20.8%, 0.262 g of the target compound 63-6 with a yield of 19.6%, and 0.145 g of the target compound 64-6 with a yield of 11.3%.

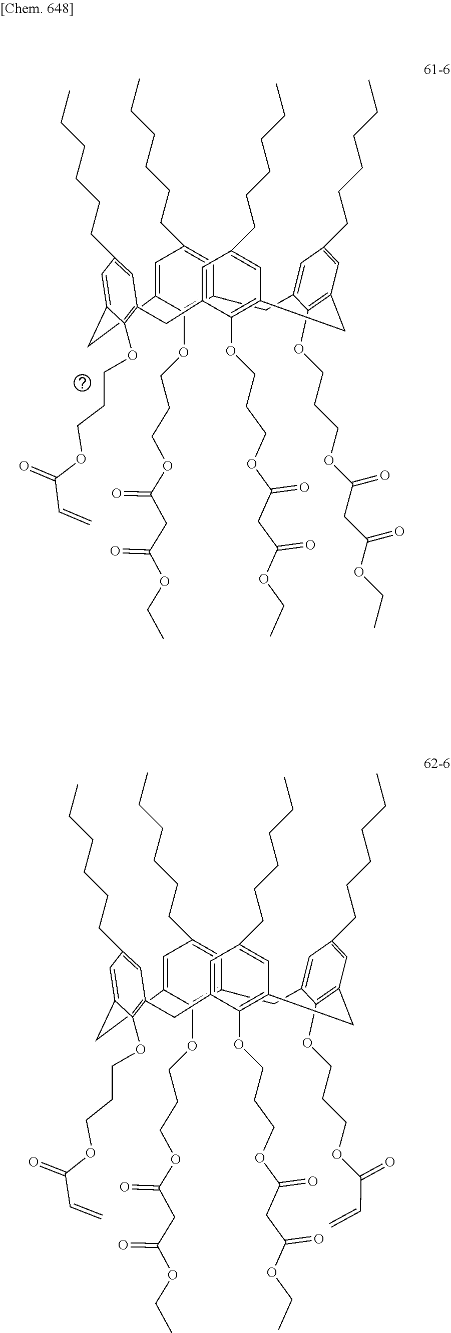 [SMILES: C=CC(=O)OCCCOc1c2cc(CCCCCC)cc1Cc1cc(CCCCCC)cc(c1OCCCOC(=O)CC(=O)OCC)Cc1cc(CCCCCC)cc(c1OCCCOC(=O)CC(=O)OCC)Cc1cc(CCCCCC)cc(c1OCCCOC(=O)CC(=O)OCC)C2 ; C=CC(=O)OCCCOc1c2cc(CCCCCC)cc1Cc1cc(CCCCCC)cc(c1OCCCOC(=O)CC(=O)OCC)Cc1cc(CCCCCC)cc(c1OCCCOC(=O)C=C)Cc1cc(CCCCCC)cc(c1OCCCOC(=O)CC(=O)OCC)C2]

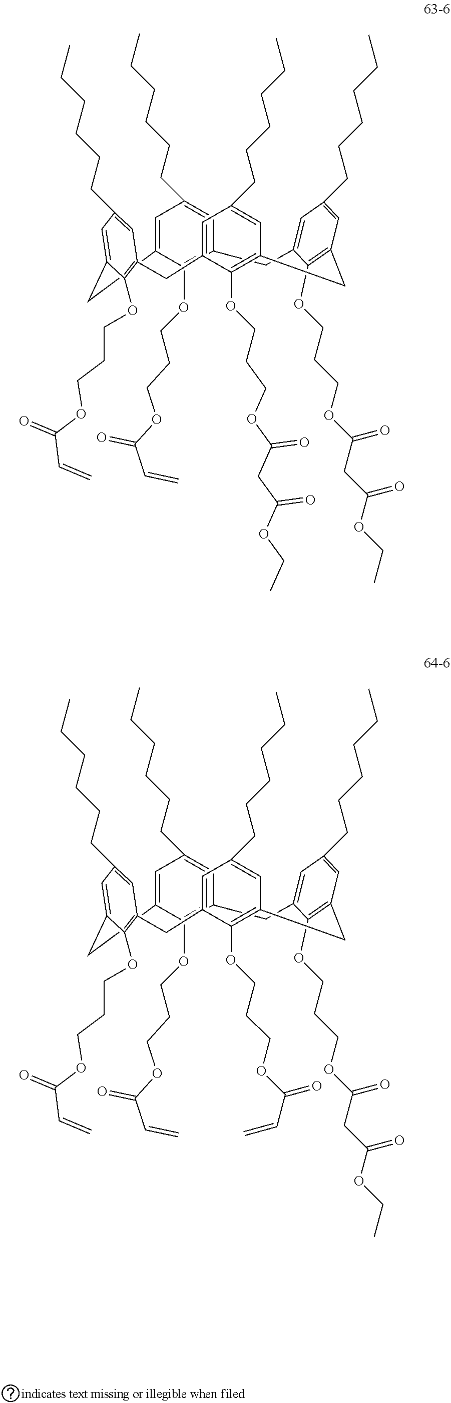 [SMILES: C=CC(=O)OCCCOc1c2cc(CCCCCC)cc1Cc1cc(CCCCCC)cc(c1OCCCOC(=O)CC(=O)OCC)Cc1cc(CCCCCC)cc(c1OCCCOC(=O)CC(=O)OCC)Cc1cc(CCCCCC)cc(c1OCCCOC(=O)C=C)C2 ; C=CC(=O)OCCCOc1c2cc(CCCCCC)cc1Cc1cc(CCCCCC)cc(c1OCCCOC(=O)C=C)Cc1cc(CCCCCC)cc(c1OCCCOC(=O)CC(=O)OCC)Cc1cc(CCCCCC)cc(c1OCCCOC(=O)C=C)C2]

### Synthesis Example 32

A 50 mL four-necked flask equipped with a stirrer, a thermometer, and a reflux condenser tube was charged with 2.00 g (2.424 mmol) of I-6, 10.00 g of tetrahydrofuran, 1.2716 g (4.848 mmol) of triphenylphosphine, and 1.024 g (4.732 mmol) of 2-[[[(1,1-dimethylethyl)dimethylsilyl]oxy]-2-propenoic acid, and the mixture was stirred. A light yellow transparent solution was thereby obtained. Then 0.9803 g (4.848 mmol) of diisopropyl azodicarboxylate was added dropwise to the mixture placed in an ice bath over 30 minutes. The light yellow transparent solution remained unchanged. The solution was stirred at room temperature for 6 hours. Hexane was added to the reaction solution to precipitate and remove by-products such as triphenylphosphine. The resulting solution was extracted with chloroform, washed with water and saturated brine, and dried over magnesium sulfate. The solvent was removed by evaporation using an evaporator, and the resulting red viscous liquid was purified by column chromatography (eluent: n-hexane:acetone=95:5) to thereby obtain a light yellow transparent liquid. The solvent was concentrated, and chloroform/methanol were added to reprecipitate crystals. The white crystals were filtered on a Kiriyama funnel, and the obtained white crystals were vacuum dried (at 60° C. for 6 hours or longer) to thereby obtain 1.891 g of compound M-6 represented by the following structural formula. The yield was 48.2%.

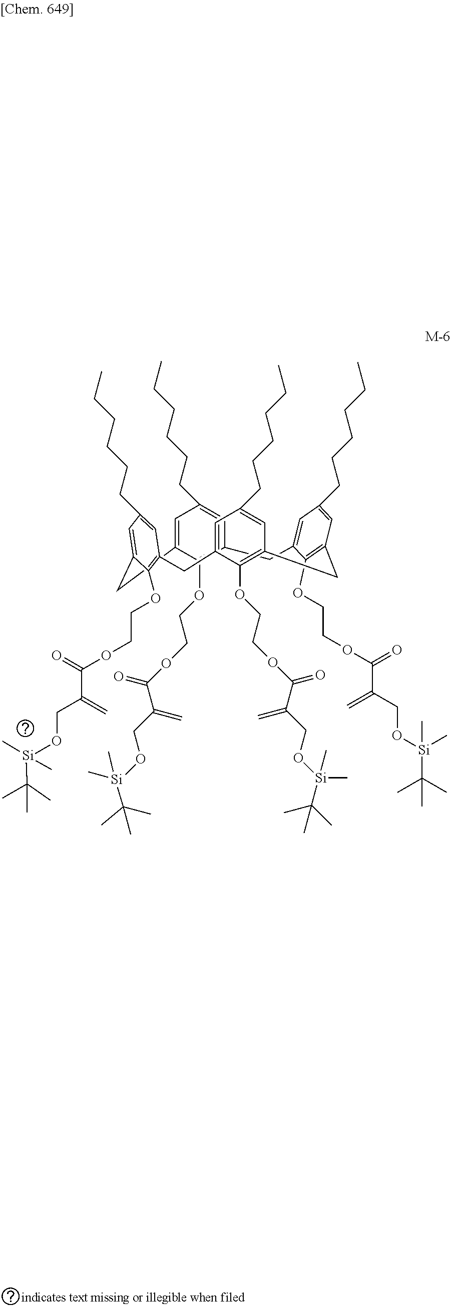 [SMILES: C=C(CO[Si](C)(C)C(C)(C)C)C(=O)OCCOc1c2cc(CCCCCC)cc1Cc1cc(CCCCCC)cc(c1OCCOC(=O)C(=C)CO[Si](C)(C)C(C)(C)C)Cc1cc(CCCCCC)cc(c1OCCOC(=O)C(=C)CO[Si](C)(C)C(C)(C)C)Cc1cc(CCCCCC)cc(c1OCCOC(=O)C(=C)CO[Si](C)(C)C(C)(C)C)C2]

### Synthesis Example 33

The same procedure as in Synthesis Example 32 was repeated except that I-4 was used instead of I-6, to thereby obtain 1.641 g of compound M-4 represented by the following structural formula. The yield was 57.3%.

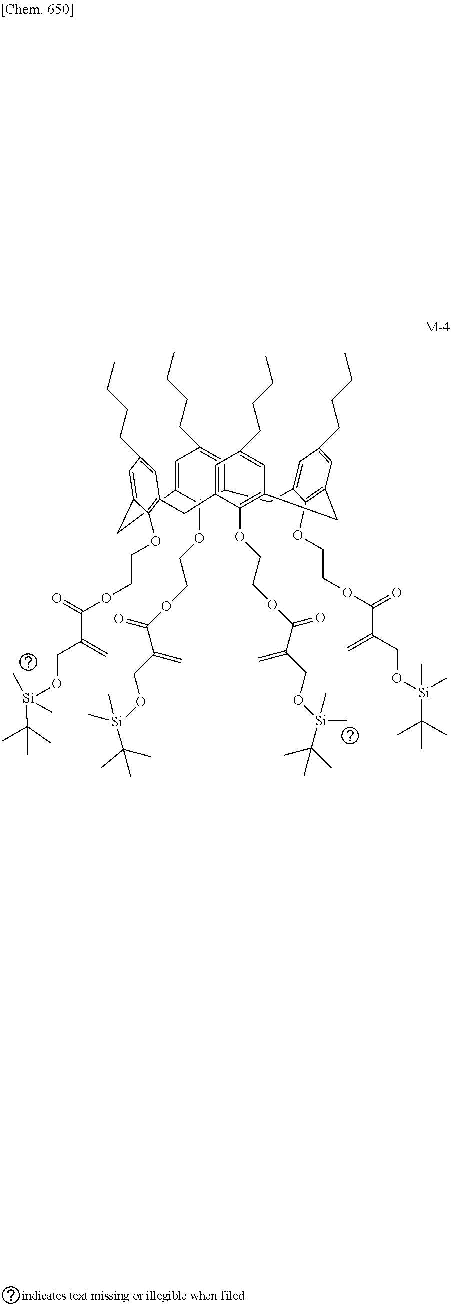 [SMILES: C=C(CO[Si](C)(C)C(C)(C)C)C(=O)OCCOc1c2cc(CCCC)cc1Cc1cc(CCCC)cc(c1OCCOC(=O)C(=C)CO[Si](C)(C)C(C)(C)C)Cc1cc(CCCC)cc(c1OCCOC(=O)C(=C)CO[Si](C)(C)C(C)(C)C)Cc1cc(CCCC)cc(c1OCCOC(=O)C(=C)CO[Si](C)(C)C(C)(C)C)C2]

### Synthesis Example 34

The same procedure as in Synthesis Example 32 was repeated except that I-7 was used instead of I-6, to thereby obtain 1.880 g of compound M-7 represented by the following structural formula. The yield was 79.0%.

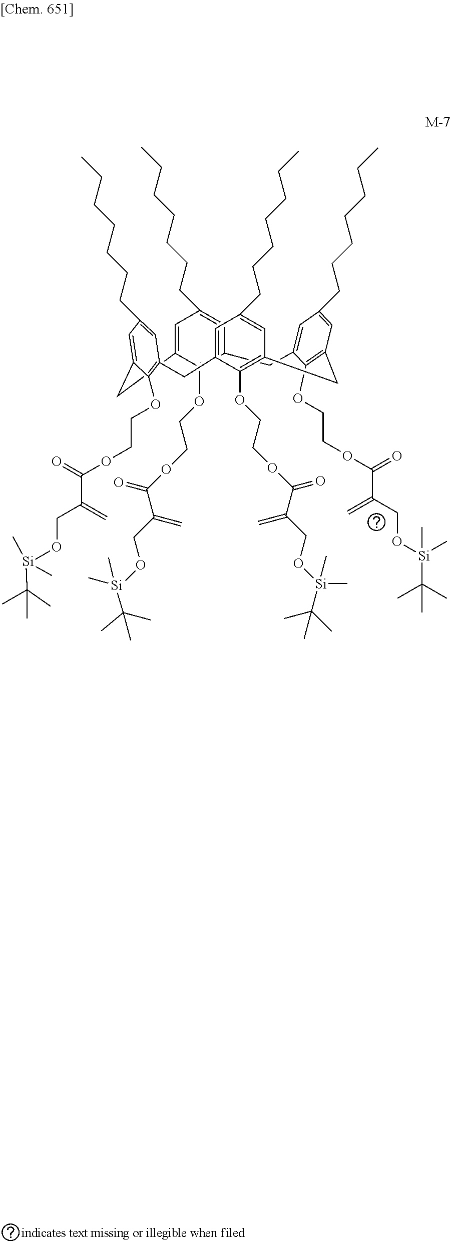 [SMILES: C=C(CO[Si](C)(C)C(C)(C)C)C(=O)OCCOc1c2cc(CCCCCCC)cc1Cc1cc(CCCCCCC)cc(c1OCCOC(=O)C(=C)CO[Si](C)(C)C(C)(C)C)Cc1cc(CCCCCCC)cc(c1OCCOC(=O)C(=C)CO[Si](C)(C)C(C)(C)C)Cc1cc(CCCCCCC)cc(c1OCCOC(=O)C(=C)CO[Si](C)(C)C(C)(C)C)C2]

### Synthesis Example 35

The same procedure as in Synthesis Example 32 was repeated except that I-18 was used instead of I-6, to thereby obtain 2.132 g of compound M-18 represented by the following structural formula. The yield was 71.4%.

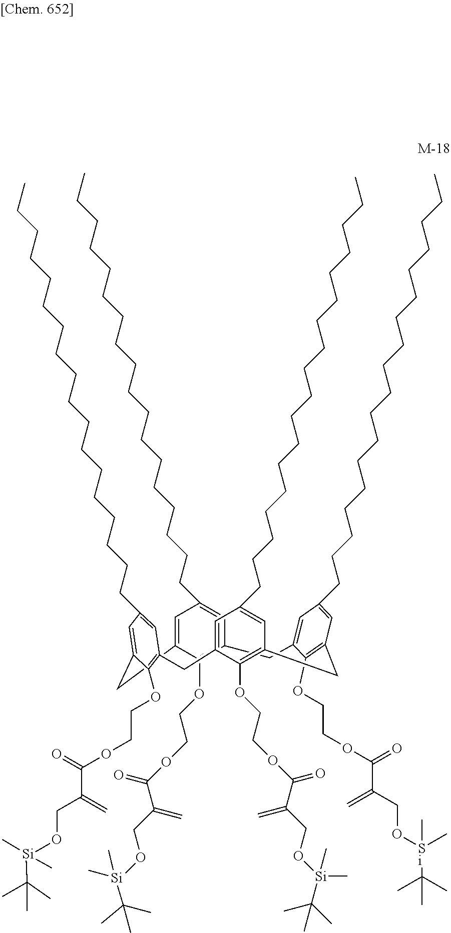 [SMILES: C=C(CO[Si](C)(C)C(C)(C)C)C(=O)OCCOc1c2cc(CCCCCCCCCCCCCCCCCC)cc1Cc1cc(CCCCCCCCCCCCCCCCCC)cc(c1OCCOC(=O)C(=C)CO[Si](C)(C)C(C)(C)C)Cc1cc(CCCCCCCCCCCCCCCCCC)cc(c1OCCOC(=O)C(=C)CO[Si](C)(C)C(C)(C)C)Cc1cc(CCCCCCCCCCCCCCCCCC)cc(c1OCCOC(=O)C(=C)CO[Si](C)(C)C(C)(C)C)C2]

### Synthesis Example 36

The same procedure as in Synthesis Example 32 was repeated except that I-1 was used instead of I-6, to thereby obtain 1.762 g of compound M-1 represented by the following structural formula. The yield was 39.9%.

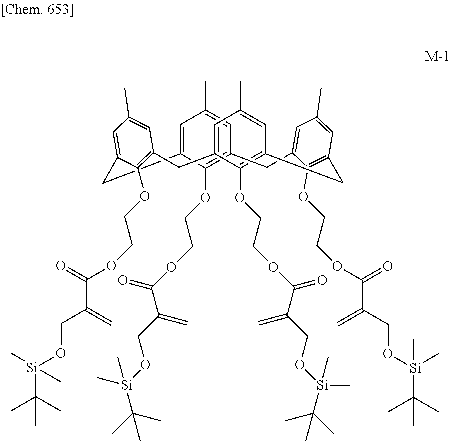 [SMILES: C=C(CO[Si](C)(C)C(C)(C)C)C(=O)OCCOc1c2cc(C)cc1Cc1cc(C)cc(c1OCCOC(=O)C(=C)CO[Si](C)(C)C(C)(C)C)Cc1cc(C)cc(c1OCCOC(=O)C(=C)CO[Si](C)(C)C(C)(C)C)Cc1cc(C)cc(c1OCCOC(=O)C(=C)CO[Si](C)(C)C(C)(C)C)C2]

### Synthesis Example 37

A 100 mL four-necked flask equipped with a stirrer, a thermometer, and a reflux condenser tube was charged with 1.891 g (1.168 mmol) of M-6, 50.00 g of tetrahydrofuran, and 0.3367 g (5.606 mmol) of acetic acid, and the mixture was stirred. Then tetrabutylammonium fluoride (an about 1 mol/L tetrahydrofuran solution; 5.61 ml (5.61 mmol)) was slowly added dropwise to the mixture placed in an ice bath. The resulting light yellow transparent reaction solution was stirred at room temperature for 6 hours. Ion exchanged water was added to the solution placed in an ice bath to stop the reaction, and 30 g of chloroform was added. Then the reaction mixture was transferred to a separatory funnel, and the organic layer was separated. Next, the aqueous layer was extracted with 30 g of chloroform three times, and the extract was combined with the organic layer. The organic layer was pre-dried over anhydrous magnesium sulfate, and the solvent was removed by evaporation using an evaporator to thereby obtain a red transparent liquid. The red transparent liquid was purified by column chromatography (eluent: n-hexane:acetone=95:5), and chloroform/methanol were added to the resulting light yellow transparent liquid to reprecipitate crystals. The white crystals were filtered on a Kiriyama funnel and vacuum dried (at 60° C. for 6 hours or longer) to thereby obtain 0.8451 g of compound N-6 represented by the following structural formula. The yield was 62.3%.

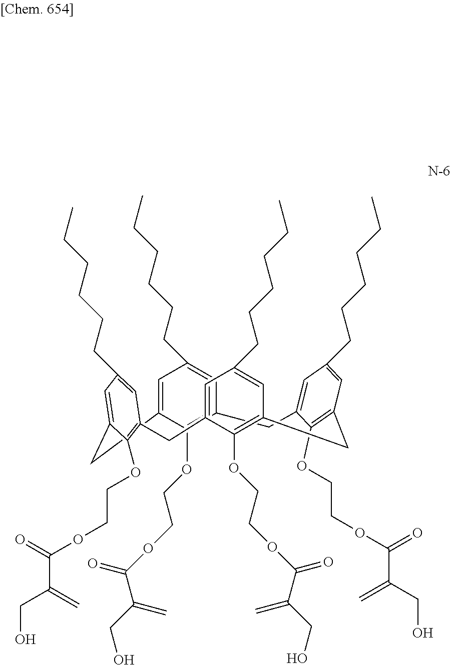 [SMILES: C=C(CO)C(=O)OCCOc1c2cc(CCCCCC)cc1Cc1cc(CCCCCC)cc(c1OCCOC(=O)C(=C)CO)Cc1cc(CCCCCC)cc(c1OCCOC(=O)C(=C)CO)Cc1cc(CCCCCC)cc(c1OCCOC(=O)C(=C)CO)C2]

### Synthesis Example 38

The same procedure as in Synthesis Example 37 was repeated except that M-4 was used instead of M-6, to thereby obtain 0.639 g of compound N-4 represented by the following structural formula. The yield was 54.3%.

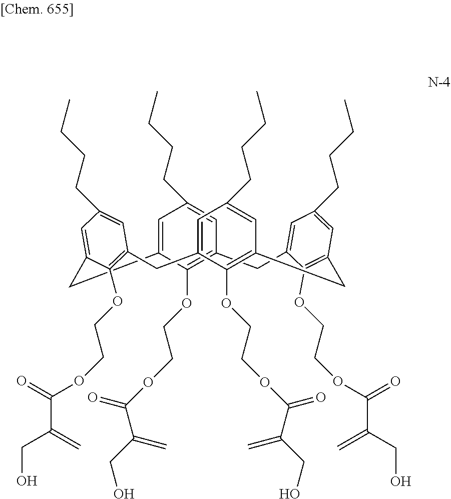 [SMILES: C=C(CO)C(=O)OCCOc1c2cc(CCCC)cc1Cc1cc(CCCC)cc(c1OCCOC(=O)C(=C)CO)Cc1cc(CCCC)cc(c1OCCOC(=O)C(=C)CO)Cc1cc(CCCC)cc(c1OCCOC(=O)C(=C)CO)C2]

### Synthesis Example 39

The same procedure as in Synthesis Example 37 was repeated except that M-7 was used instead of M-6, to thereby obtain 0.873 g of compound N-7 represented by the following structural formula. The yield was 62.4%.

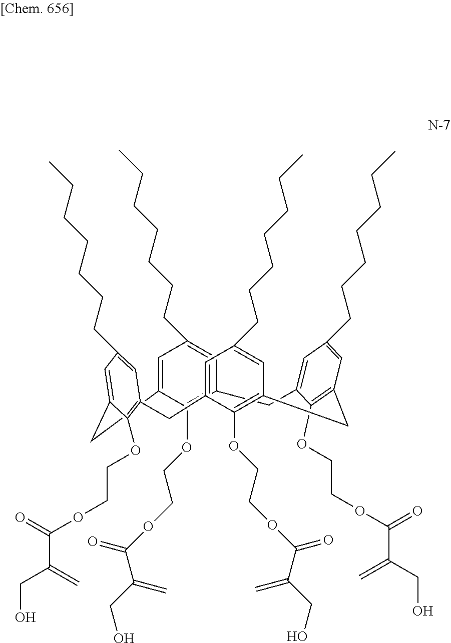 [SMILES: C=C(CO)C(=O)OCCOc1c2cc(CCCCCCC)cc1Cc1cc(CCCCCCC)cc(c1OCCOC(=O)C(=C)CO)Cc1cc(CCCCCCC)cc(c1OCCOC(=O)C(=C)CO)Cc1cc(CCCCCCC)cc(c1OCCOC(=O)C(=C)CO)C2]

### Synthesis Example 40

The same procedure as in Synthesis Example 37 was repeated except that M-18 was used instead of M-6, to thereby obtain 1.092 g of compound N-18 represented by the following structural formula. The yield was 63.2%.

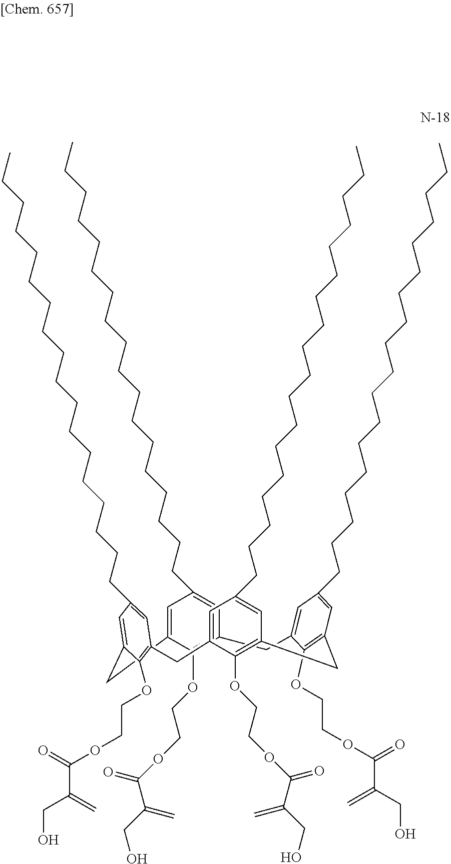 [SMILES: C=C(CO)C(=O)OCCOc1c2cc(CCCCCCCCCCCCCCCCCC)cc1Cc1cc(CCCCCCCCCCCCCCCCCC)cc(c1OCCOC(=O)C(=C)CO)Cc1cc(CCCCCCCCCCCCCCCCCC)cc(c1OCCOC(=O)C(=C)CO)Cc1cc(CCCCCCCCCCCCCCCCCC)cc(c1OCCOC(=O)C(=C)CO)C2]

### Synthesis Example 41

The same procedure as in Synthesis Example 37 was repeated except that M-1 was used instead of M-6, to thereby obtain 0.654 g of compound N-1 represented by the following structural formula. The yield was 54.2%.

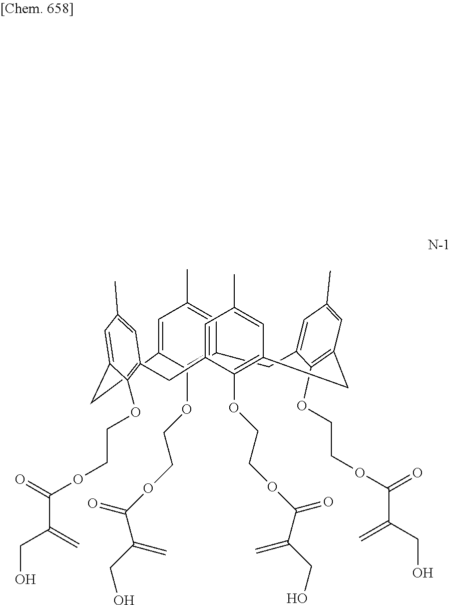 [SMILES: C=C(CO)C(=O)OCCOc1c2cc(C)cc1Cc1cc(C)cc(c1OCCOC(=O)C(=C)CO)Cc1cc(C)cc(c1OCCOC(=O)C(=C)CO)Cc1cc(C)cc(c1OCCOC(=O)C(=C)CO)C2]

### Example 25

A 30 mL four-necked flask equipped with a stirrer, a thermometer, and a reflux condenser tube was charged with 0.300 g (0.236 mmol) of N-6, 0.679 g of tetrahydrofuran, 0.494 g (1.884 mmol) of triphenylphosphine, and 0.223 g (1.884 mmol) of monomethyl malonate, and the mixture was stirred. Then 0.423 g (1.884 mmol) of diisopropyl azodicarboxylate diluted with 0.340 g of tetrahydrofuran was added dropwise to the mixture placed in an ice bath over 30 minutes, The resulting light yellow transparent reaction solution was stirred at room temperature for 6 hours. Hexane was added to the reaction solution to precipitate and remove by-products such as triphenylphosphine, and the resulting solution was extracted with chloroform. The resulting solution was washed with water and saturated brine and dried over magnesium sulfate, and the solvent was removed by evaporation using an evaporator to thereby obtain a red viscous liquid. The red viscous liquid was purified by column chromatography (eluent: n-hexane:ethyl acetate=85:15) to thereby obtain 0.311 g of the target compound 65-6. The yield was 78.9%.

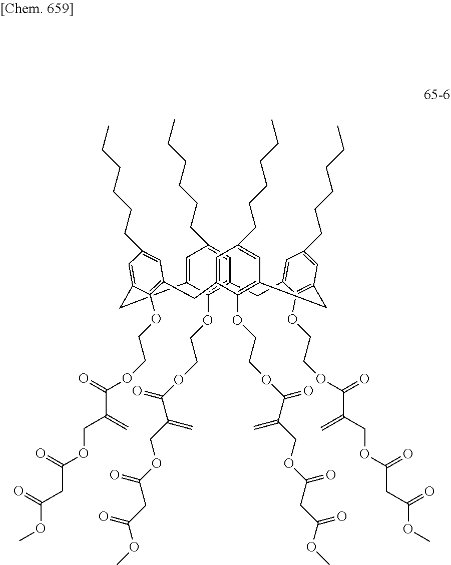 [SMILES: C=C(COC(=O)CC(=O)OC)C(=O)OCCOc1c2cc(CCCCCC)cc1Cc1cc(CCCCCC)cc(c1OCCOC(=O)C(=C)COC(=O)CC(=O)OC)Cc1cc(CCCCCC)cc(c1OCCOC(=O)C(=C)COC(=O)CC(=O)OC)Cc1cc(CCCCCC)cc(c1OCCOC(=O)C(=C)COC(=O)CC(=O)OC)C2]

### Example 26

The same procedure as in Synthesis Example 25 was repeated except that N-4 was used instead of N-6, to thereby obtain 0.301 g of the target compound 65-4. The yield was 74.6%.

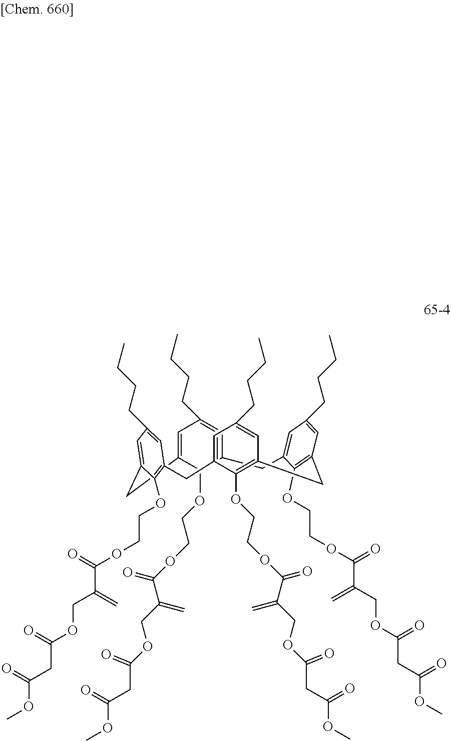 [SMILES: C=C(COC(=O)CC(=O)OC)C(=O)OCCOc1c2cc(CCCC)cc1Cc1cc(CCCC)cc(c1OCCOC(=O)C(=C)COC(=O)CC(=O)OC)Cc1cc(CCCC)cc(c1OCCOC(=O)C(=C)COC(=O)CC(=O)OC)Cc1cc(CCCC)cc(c1OCCOC(=O)C(=C)COC(=O)CC(=O)OC)C2]

### Example 27

The same procedure as in Example 25 was repeated except that N-7 was used instead of N-6, to thereby obtain 0.311 g of the target compound 65-7. The yield was 79.7%.

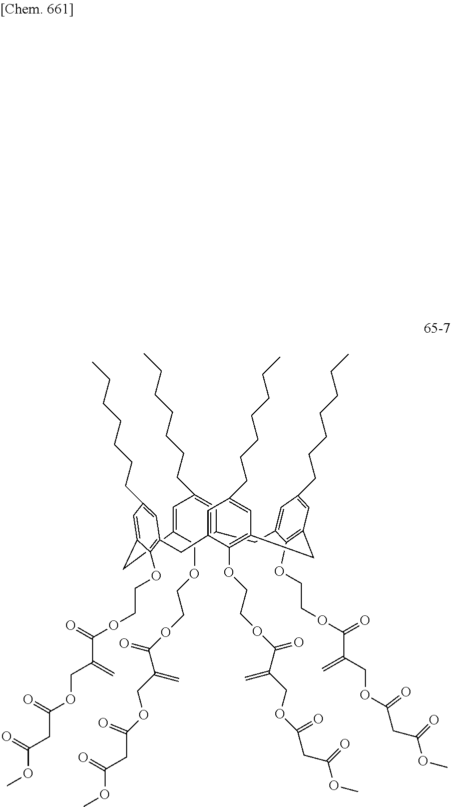

### Example 28

The same procedure as in Example 25 was repeated except that N-18 was used instead of N-6, to thereby obtain 0.303 g of the target compound 65-18. The yield was 83.8%.

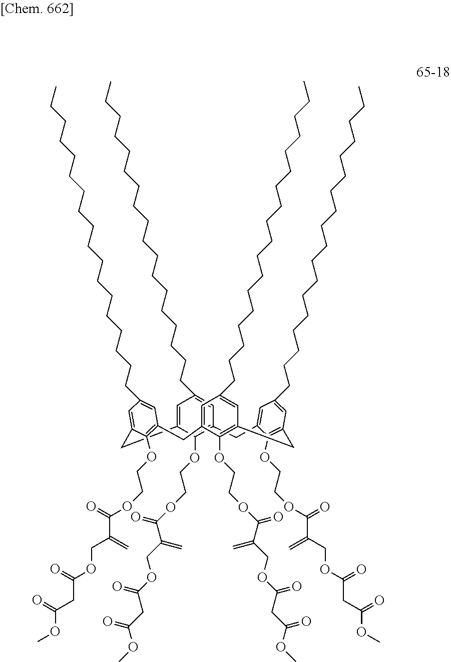 [SMILES: C=C(COC(=O)CC(=O)OC)C(=O)OCCOc1c2cc(CCCCCCCCCCCCCCCCCC)cc1Cc1cc(CCCCCCCCCCCCCCCCCC)cc(c1OCCOC(=O)C(=C)COC(=O)CC(=O)OC)Cc1cc(CCCCCCCCCCCCCCCCCC)cc(c1OCCOC(=O)C(=C)COC(=O)CC(=O)OC)Cc1cc(CCCCCCCCCCCCCCCCCC)cc(c1OCCOC(=O)C(=C)COC(=O)CC(=O)OC)C2]

### Example 29

The same procedure as in Example 25 was repeated except that N-1 was used instead of N-6, to thereby obtain 0.295 g of the target compound 65-1. The yield was 70.1%.

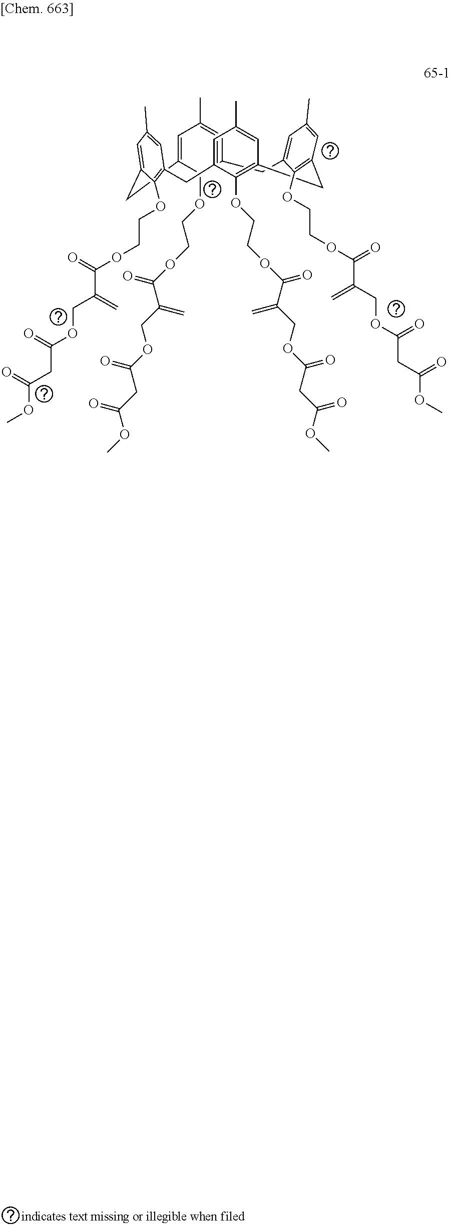 [SMILES: C=C(COC(=O)CC(=O)OC)C(=O)OCCOc1c2cc(C)cc1Cc1cc(C)cc(c1OCCOC(=O)C(=C)COC(=O)CC(=O)OC)Cc1cc(C)cc(c1OCCOC(=O)C(=C)COC(=O)CC(=O)OC)Cc1cc(C)cc(c1OCCOC(=O)C(=C)COC(=O)CC(=O)OC)C2]

### Example 30

The same procedure as in Example 25 was repeated except that monoethyl malonate was used instead of monomethyl malonate, to thereby obtain 0.338 g of the target compound 66-6. The yield was 82.9%.

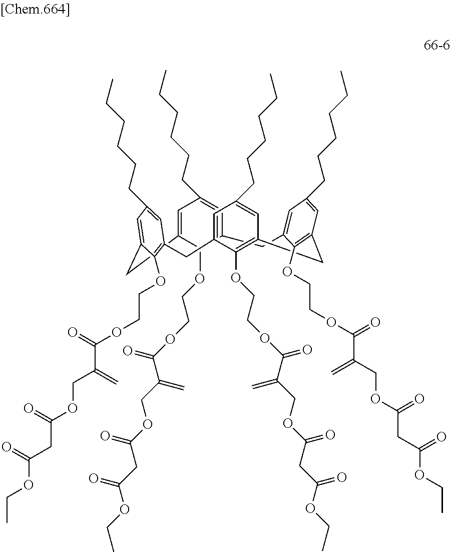 [SMILES: C=C(COC(=O)CC(=O)OCC)C(=O)OCCOc1c2cc(CCCCCC)cc1Cc1cc(CCCCCC)cc(c1OCCOC(=O)C(=C)COC(=O)CC(=O)OCC)Cc1cc(CCCCCC)cc(c1OCCOC(=O)C(=C)COC(=O)CC(=O)OCC)Cc1cc(CCCCCC)cc(c1OCCOC(=O)C(=C)COC(=O)CC(=O)OCC)C2]

### Synthesis Example 42

The same procedure as in Synthesis Example 32 was repeated except that 4-[[[(1,1-dimethylethyl)dimethylsilyl]oxy]-2-methylenebutanoic acid was used instead of 2-[[[(1,1-dimethylethyl)dimethylsilyl]oxy]2-propenoic acid, to thereby obtain 2.420 g of compound O-6 represented by the following structural formula. The yield was 72.6%.

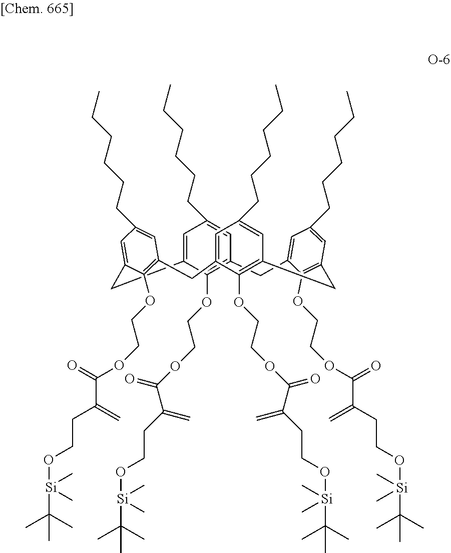 [SMILES: C=C(CCO[Si](C)(C)C(C)(C)C)C(=O)OCCOc1c2cc(CCCCCC)cc1Cc1cc(CCCCCC)cc(c1OCCOC(=O)C(=C)CCO[Si](C)(C)C(C)(C)C)Cc1cc(CCCCCC)cc(c1OCCOC(=O)C(=C)CCO[Si](C)(C)C(C)(C)C)Cc1cc(CCCCCC)cc(c1OCCOC(=O)C(=C)CCO[Si](C)(C)C(C)(C)C)C2]

### Synthesis Example 43

The same procedure as in Synthesis Example 37 was repeated except that O-6 was used instead of M-6, to thereby obtain 1.07 g of compound P-6 represented by the following structural formula. The yield was 59.4%.

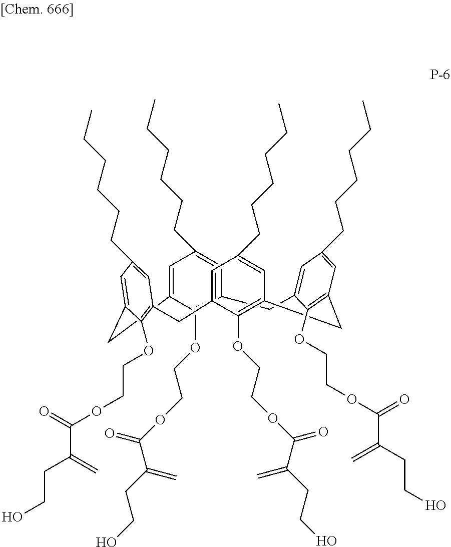 [SMILES: C=C(CCO)C(=O)OCCOc1c2cc(CCCCCC)cc1Cc1cc(CCCCCC)cc(c1OCCOC(=O)C(=C)CCO)Cc1cc(CCCCCC)cc(c1OCCOC(=O)C(=C)CCO)Cc1cc(CCCCCC)cc(c1OCCOC(=O)C(=C)CCO)C2]

### Example 31

The same procedure as in Example 25 was repeated except that P-6 was used instead of N-6, to thereby obtain 0.299 g of the target compound 67-6. The yield was 76.6%.

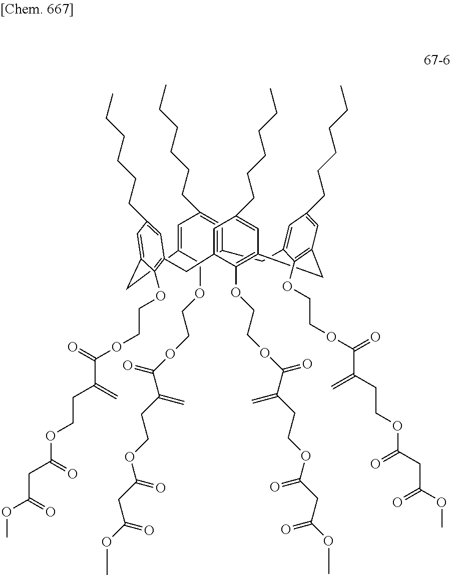 [SMILES: C=C(CCOC(=O)CC(=O)OC)C(=O)OCCOc1c2cc(CCCCCC)cc1Cc1cc(CCCCCC)cc(c1OCCOC(=O)C(=C)CCOC(=O)CC(=O)OC)Cc1cc(CCCCCC)cc(c1OCCOC(=O)C(=C)CCOC(=O)CC(=O)OC)Cc1cc(CCCCCC)cc(c1OCCOC(=O)C(=C)CCOC(=O)CC(=O)OC)C2]

### Example 32

The same procedure as in Example 31 was repeated except that monoethyl malonate was used instead of monomethyl malonate, to thereby obtain 0.317 g of the target compound 68-6. The yield was 78.6%.

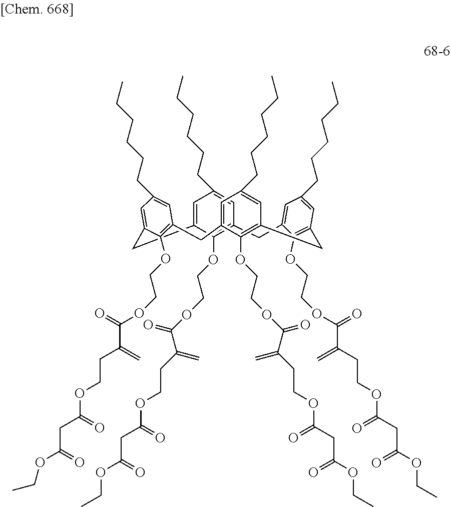 [SMILES: C=C(CCOC(=O)CC(=O)OCC)C(=O)OCCOc1c2cc(CCCCCC)cc1Cc1cc(CCCCCC)cc(c1OCCOC(=O)C(=C)CCOC(=O)CC(=O)OCC)Cc1cc(CCCCCC)cc(c1OCCOC(=O)C(=C)CCOC(=O)CC(=O)OCC)Cc1cc(CCCCCC)cc(c1OCCOC(=O)C(=C)CCOC(=O)CC(=O)OCC)C2]

### Synthesis Example 44

A 1 L four-necked flask equipped with a stirrer, a dropping funnel, a thermometer, and a reflux condenser tube was charged, in a nitrogen atmosphere, with sodium hydride (7.54 g, 188.4 mmol), and mineral oil was removed by washing with hexane. Then dry DMF (160 mL) and hexyl bromide (37.2 g, 207.4 mmol) were added, and the resulting mixture was heated to 70° C. under stirring. A solution prepared by dissolving intermediate A (10 g, 23.6 mmol) obtained in Synthesis Example 1 in dry DMF (80 mL) was added to the mixture from a dropping funnel. After completion of the addition, the resulting mixture was continuously stirred for additional 2 hours. After cooled to room temperature, the reaction mixture was poured onto ice (300 g), and concentrated hydrochloric acid was added to acidify the aqueous solution. The resulting solution was extracted with chloroform (200 mL) twice. The chloroform solution was washed with water until the pH was 5 or higher, further washed with saturated brine, and dried over anhydrous magnesium sulfate. The solvent was removed using an evaporator to thereby obtain a yellow liquid. Methanol was added to the solution under stirring to precipitate solids. The solids were collected by filtration and recrystallized with isopropyl alcohol. The obtained white crystals were vacuum dried to thereby obtain a compound represented by the following formula (11.6 g, yield 65%).

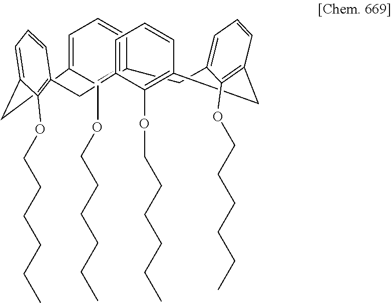 [SMILES: CCCCCCOc1c2cccc1Cc1cccc(c1OCCCCCC)Cc1cccc(c1OCCCCCC)Cc1cccc(c1OCCCCCC)C2]

### Synthesis Example 45

The same procedure as in Synthesis Example 44 was repeated except that methyl iodide was used instead of hexyl bromide and that the reaction was performed at room temperature for 24 hours, to thereby obtain a compound represented by the following formula (6.8 g, yield 60%).

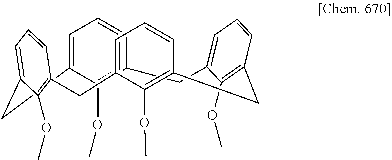 [SMILES: COc1c2cccc1Cc1cccc(c1OC)Cc1cccc(c1OC)Cc1cccc(c1OC)C2]

### Synthesis Example 46

The same procedure as in Synthesis Example 44 was repeated except that butyl bromide was used instead of hexyl bromide, to thereby obtain a compound represented by the following formula (11.0 g, yield 72%).

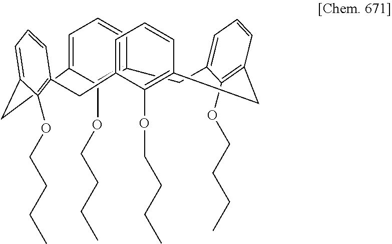 [SMILES: CCCCOc1c2cccc1Cc1cccc(c1OCCCC)Cc1cccc(c1OCCCC)Cc1cccc(c1OCCCC)C2]

### Synthesis Example 47

The same procedure as in Synthesis Example 44 was repeated except that heptyl bromide was used instead of hexyl bromide, to thereby obtain a compound represented by the following formula (14.4 g, yield 75%).

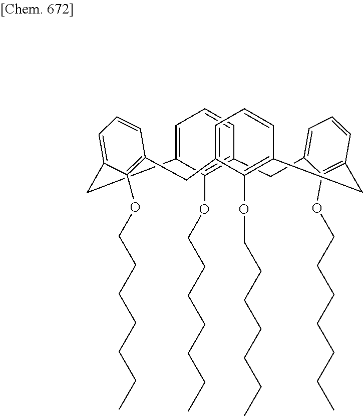 [SMILES: CCCCCCCOc1c2cccc1Cc1cccc(c1OCCCCCCC)Cc1cccc(c1OCCCCCCC)Cc1cccc(c1OCCCCCCC)C2]

### Synthesis Example 48

The same procedure as in Synthesis Example 44 was repeated except that octadecyl bromide was used instead of hexyl bromide, to thereby obtain a compound represented by the following formula (23.6 g, yield 70%).

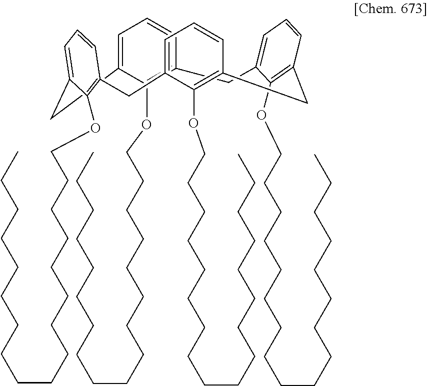 [SMILES: CCCCCCCCCCCCCCCCCCOc1c2cccc1Cc1cccc(c1OCCCCCCCCCCCCCCCCCC)Cc1cccc(c1OCCCCCCCCCCCCCCCCCC)Cc1cccc(c1OCCCCCCCCCCCCCCCCCC)C2]

### Synthesis Example 49

Referring to a known document (Organic & Biomolecular Chemistry, 13, 1708-1723; 2015), a compound represented by the following formula was synthesized in two steps using the compound obtained in Synthesis Example 44 (5.0 g, 6.57 mmol) (yield amount 3.3 g, yield 67%).

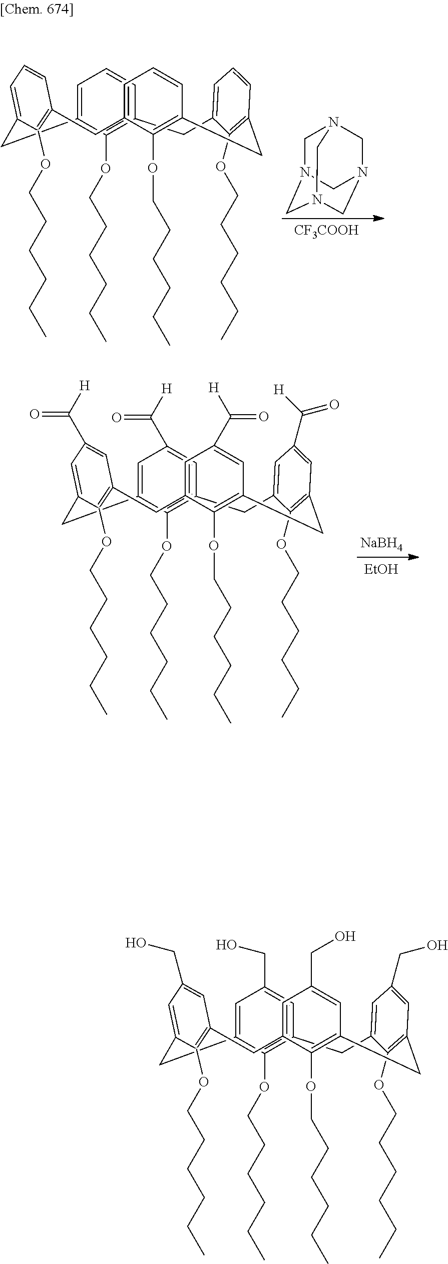

### Synthesis Example 50

The same procedure as in Synthesis Example 49 was repeated except that the compound obtained in Synthesis Example 45 (5.0 g, 10.4 mmol) was used instead of the compound obtained in Synthesis Example 44, to synthesize a compound represented by the following formula in two steps (3.75 g, yield 60%).

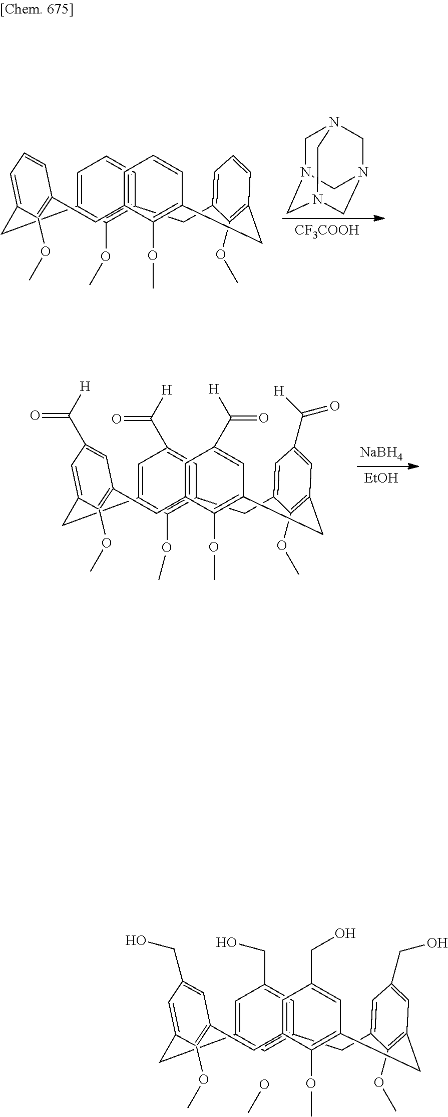

### Synthesis Example 51

The same procedure as in Synthesis Example 49 was repeated except that the compound obtained in Synthesis Example 46 (5.0 g, 7.7 mmol) was used instead of the compound obtained in Synthesis Example 44, to synthesize a compound represented by the following formula in two steps (3.73 g, yield 63%).

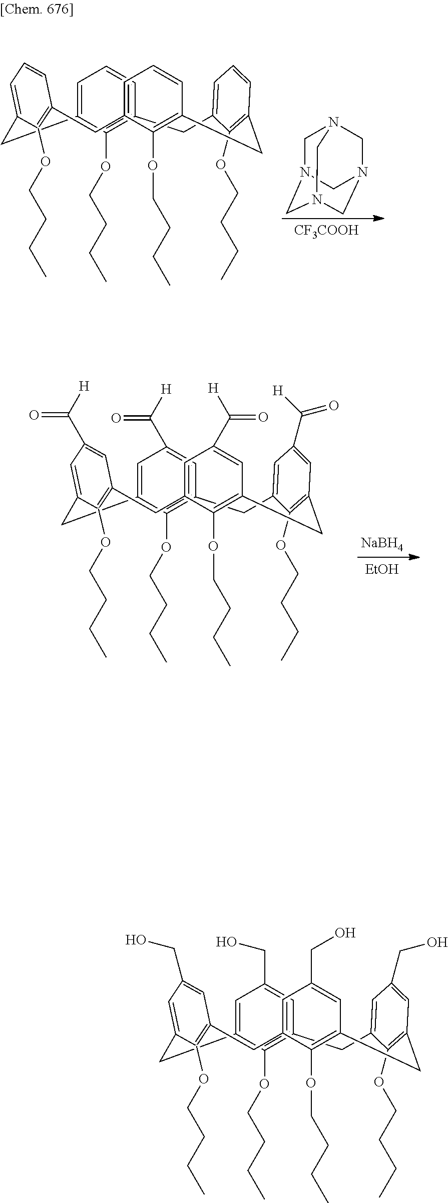

### Synthesis Example 52

The same procedure as in Synthesis Example 49 was repeated except that the compound obtained in Synthesis Example 47 (5.0 g, 6.1 mmol) was used instead of the compound obtained in Synthesis Example 4, to synthesize a compound represented by the following formula in two steps (4.01 g, yield 70%).

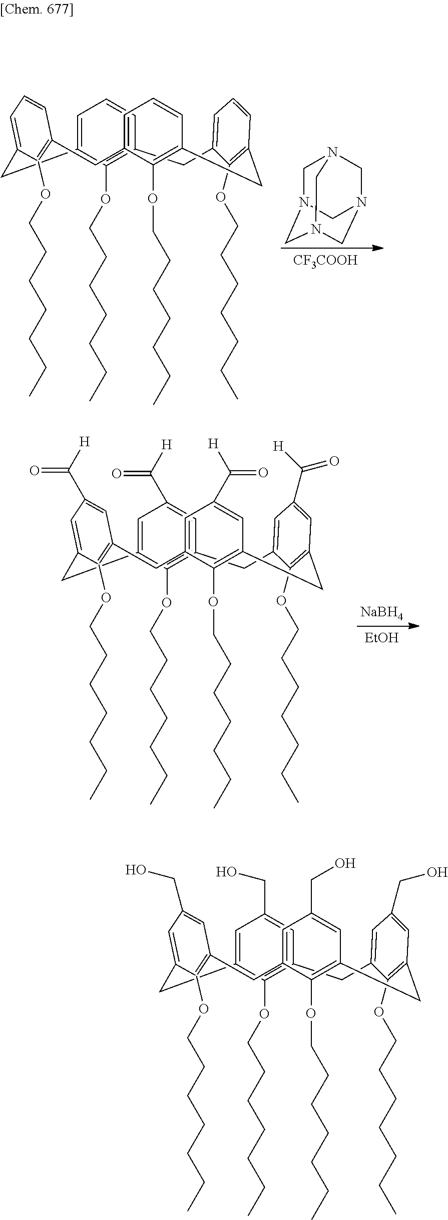

### Synthesis Example 53

The same procedure as in Synthesis Example 49 was repeated except that the compound obtained in Synthesis Example 48 (10.0 g, 7.0 mmol) was used instead of the compound obtained in Synthesis Example 44, to synthesize a compound represented by the following formula in two steps (5.96 g, yield 55%).

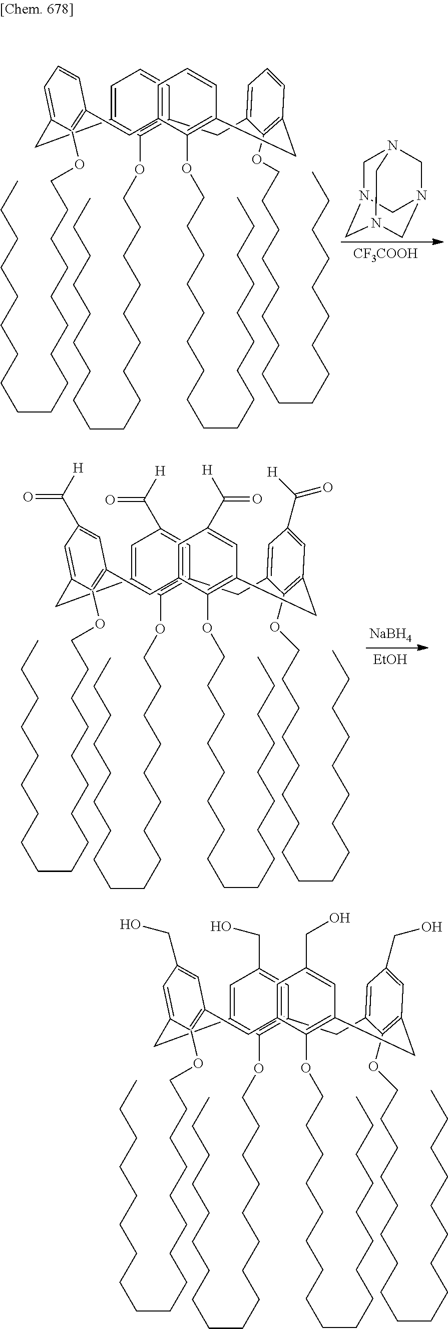

### Synthesis Example 54

A 500 mL four-necked flask equipped with a stirrer, a dropping funnel, a thermometer, and a reflux condenser tube was charged, in a nitrogen atmosphere, with sodium hydride (3.28 g, 82.1 mmol), and mineral oil was removed by washing with hexane. Then dry DMF (100 mL) and hexyl bromide (16.2 g, 90.3 mmol) were added, and the resulting mixture was heated to 70° C. under stirring. Then a solution prepared by dissolving in dry DMF (40 mL) 5,11,17,23-tetraallyl-25,26,27,28-tetrahydroxycalix[4]arene (6.0 g, 10.3 mmol) synthesized by a method described in a known document (The Journal of Organic Chemistry 50, 5802-58061; 1985) was added to the mixture from a dropping funnel. After completion of the addition, the resulting mixture was continuously stirred for additional 2 hours. The reaction mixture was cooled to room temperature and then poured onto ice (200 g). Concentrated hydrochloric acid was added to acidify the aqueous solution, and the resulting mixture was extracted with chloroform (150 mL) twice. The chloroform solution was washed with water until the pH was 5 or higher, further washed with saturated brine, and dried over anhydrous magnesium sulfate. The solvent was removed using an evaporator to thereby obtain a yellow liquid. The yellow liquid was purified by silica gel column chromatography to thereby obtain a colorless transparent solution, and then a compound represented by the following formula was obtained as a white solid by recrystallization (6.6 g, yield 70%).

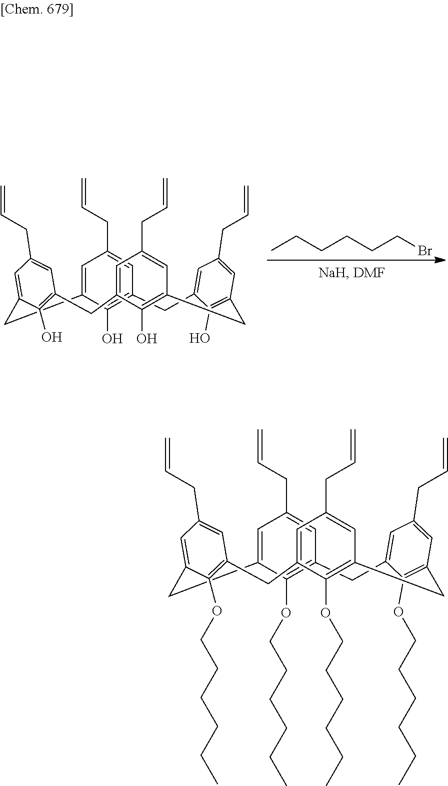

### Synthesis Example 55

The same procedure as in Synthesis Example 54 was repeated except that methyl iodide was used instead of hexyl bromide and that the reaction was performed at room temperature for 24 hours, to thereby obtain a compound represented by the following formula (4.27 g, yield 65%).

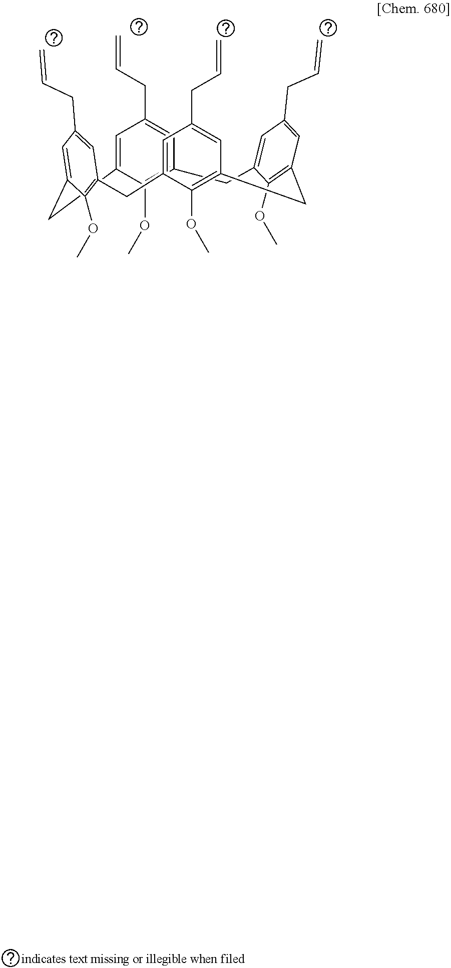 [SMILES: C=CCc1cc2c(OC)c(c1)Cc1cc(CC=C)cc(c1OC)Cc1cc(CC=C)cc(c1OC)Cc1cc(CC=C)cc(c1OC)C2]

### Synthesis Example 56

The same procedure as in Synthesis Example 54 was repeated except that butyl bromide was used instead of hexyl bromide, to thereby obtain a compound represented by the following formula (6.23 g, yield 75%).

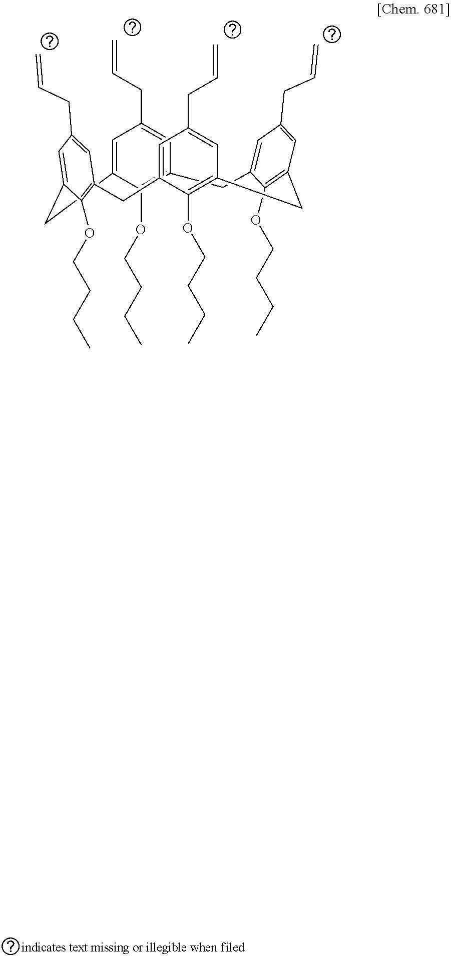 [SMILES: C=CCc1cc2c(OCCCC)c(c1)Cc1cc(CC=C)cc(c1OCCCC)Cc1cc(CC=C)cc(c1OCCCC)Cc1cc(CC=C)cc(c1OCCCC)C2]

### Synthesis Example 57

The same procedure as in Synthesis Example 54 was repeated except that heptyl bromide was used instead of hexyl bromide, to thereby obtain a compound represented by the following formula (8.02 g, yield 80%).

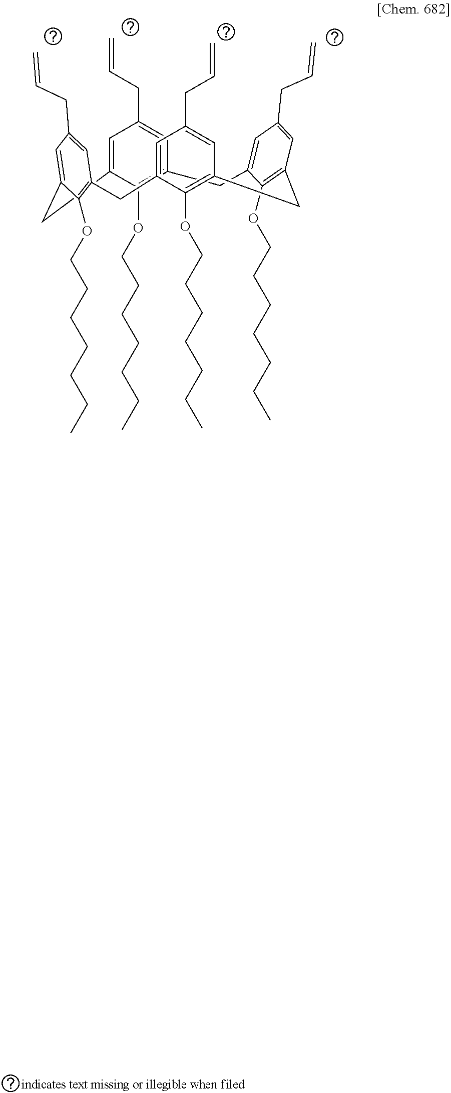 [SMILES: C=CCc1cc2c(OCCCCCCC)c(c1)Cc1cc(CC=C)cc(c1OCCCCCCC)Cc1cc(CC=C)cc(c1OCCCCCCC)Cc1cc(CC=C)cc(c1OCCCCCCC)C2]

### Synthesis Example 58

The same procedure as in Synthesis Example 54 was repeated except that octadecyl bromide was used instead of hexyl bromide, to thereby obtain a compound represented by the following formula (12.8 g, yield 75%).

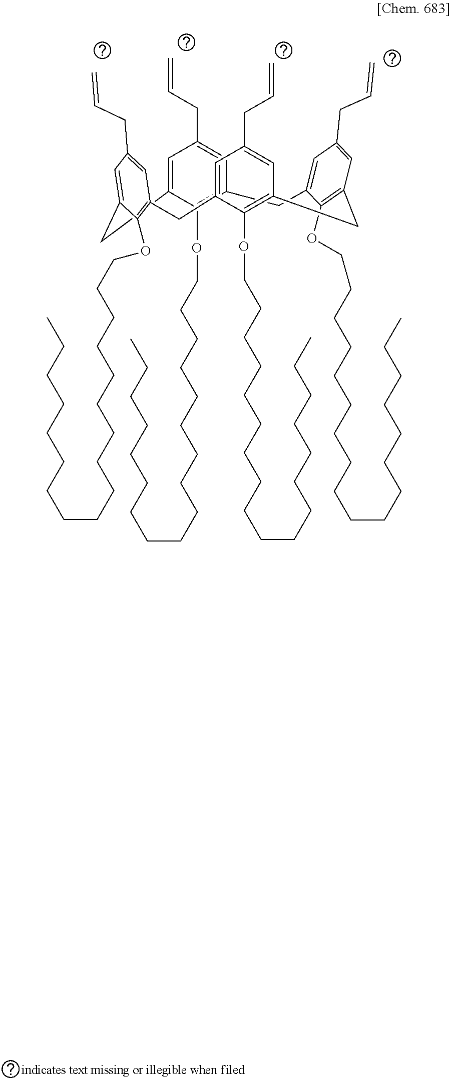 [SMILES: C=CCc1cc2c(OCCCCCCCCCCCCCCCCCC)c(c1)Cc1cc(CC=C)cc(c1OCCCCCCCCCCCCCCCCCC)Cc1cc(CC=C)cc(c1OCCCCCCCCCCCCCCCCCC)Cc1cc(CC=C)cc(c1OCCCCCCCCCCCCCCCCCC)C2]

### Synthesis Example 59

Referring to a known document (The Journal of Organic Chemistry, 67, 4722-4733; 2002), a compound represented by the following formula was synthesized using the compound obtained in Synthesis Example 54 (4 g, 4.34 mmol) (yield amount 2.93 g, yield 68%).

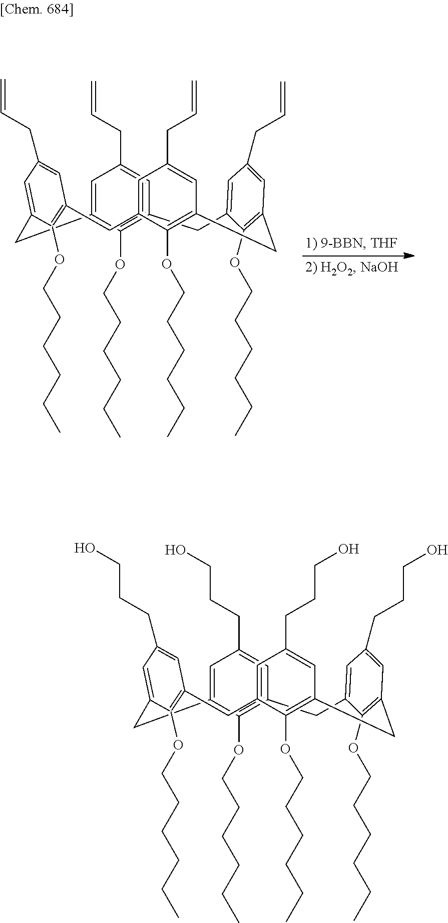

### Synthesis Example 60

The same procedure as in Synthesis Example 59 was repeated except that the compound obtained in Synthesis Example 55 (4.0 g, 6.24 mmol) was used instead of the compound obtained in Synthesis Example 54, to thereby obtain a compound represented by the following formula (4.5 g, yield 72%).

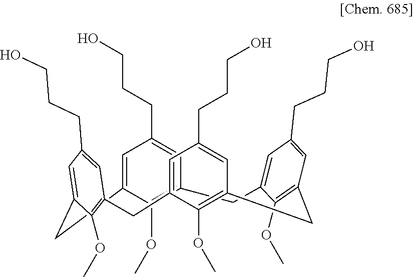 [SMILES: COc1c2cc(CCCO)cc1Cc1cc(CCCO)cc(c1OC)Cc1cc(CCCO)cc(c1OC)Cc1cc(CCCO)cc(c1OC)C2]

### Synthesis Example 61

The same procedure as in Synthesis Example 59 was repeated except that the compound obtained in Synthesis Example 56 (4.0 g, 4.94 mmol) was used instead of the compound obtained in Synthesis Example 54, to thereby obtain a compound represented by the following formula (2.59 g, yield 65%).

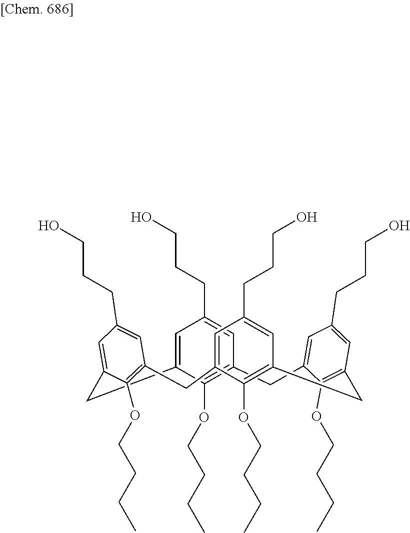 [SMILES: CCCCOc1c2cc(CCCO)cc1Cc1cc(CCCO)cc(c1OCCCC)Cc1cc(CCCO)cc(c1OCCCC)Cc1cc(CCCO)cc(c1OCCCC)C2]

### Synthesis Example 62

The same procedure as in Synthesis Example 59 was repeated except that the compound obtained in Synthesis Example 57 (4.0 g, 4.11 mmol) was used instead of the compound obtained in Synthesis Example 54, to thereby obtain a compound represented by the following formula (3.23 g, yield 75%).

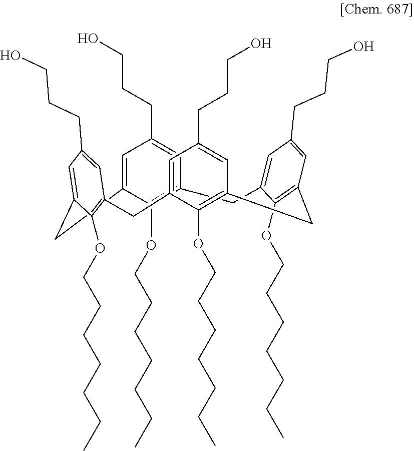 [SMILES: CCCCCCCOc1c2cc(CCCO)cc1Cc1cc(CCCO)cc(c1OCCCCCCC)Cc1cc(CCCO)cc(c1OCCCCCCC)Cc1cc(CCCO)cc(c1OCCCCCCC)C2]

### Synthesis Example 63

The same procedure as in Synthesis Example 59 was repeated except that the compound obtained in Synthesis Example 57 (8.0 g, 5.02 mmol) was used instead of the compound obtained in Synthesis Example 54, to thereby obtain a compound represented by the following formula (5.1 g, yield 61%).

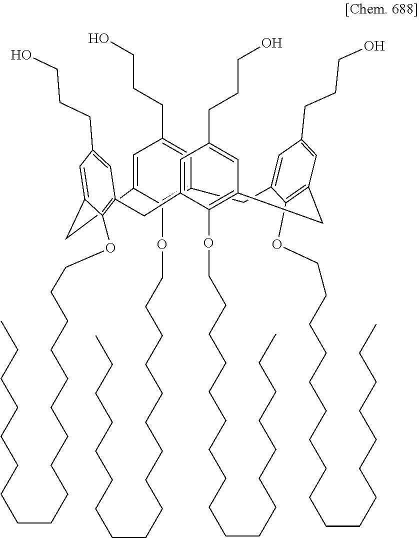 [SMILES: CCCCCCCCCCCCCCCCCCOc1c2cc(CCCO)cc1Cc1cc(CCCO)cc(c1OCCCCCCCCCCCCCCCCCC)Cc1cc(CCCO)cc(c1OCCCCCCCCCCCCCCCCCC)Cc1cc(CCCO)cc(c1OCCCCCCCCCCCCCCCCCC)C2]

### Example 33

A 100 mL four-necked flask equipped with a stirrer, a dropping funnel, and a thermometer was charged, in a nitrogen atmosphere, with the compound obtained in Synthesis Example 49 (3.0 g, 3.94 mmol), triethylamine (3.19 g, 31.52 mmol), and methylene chloride (35.5 mL), and the mixture was stirred under cooling with ice. A solution prepared by dissolving acrylic acid chloride (0.856 g, 9.46 mmol) and methyl malonyl chloride (1.291 g, 9.46 mmol) in methylene chloride (5 mL) was slowly added dropwise. After completion of the dropwise addition, the resulting mixture was stirred at room temperature for 8 hours. Water was added to the reaction mixture, and the resulting mixture was extracted with chloroform (50 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator to thereby obtain a yellow liquid. The yellow liquid was purified by silica gel column chromatography to thereby obtain the target compounds 01-6, 02-6, 03-6, and 04-6 as follows. 01-6 (0.657 g, yield 13.5%). A mixture of 02-6 and 03-6 (2.587 g, yield 55.2%). 04-6 (0.653 g, yield 14.5%).

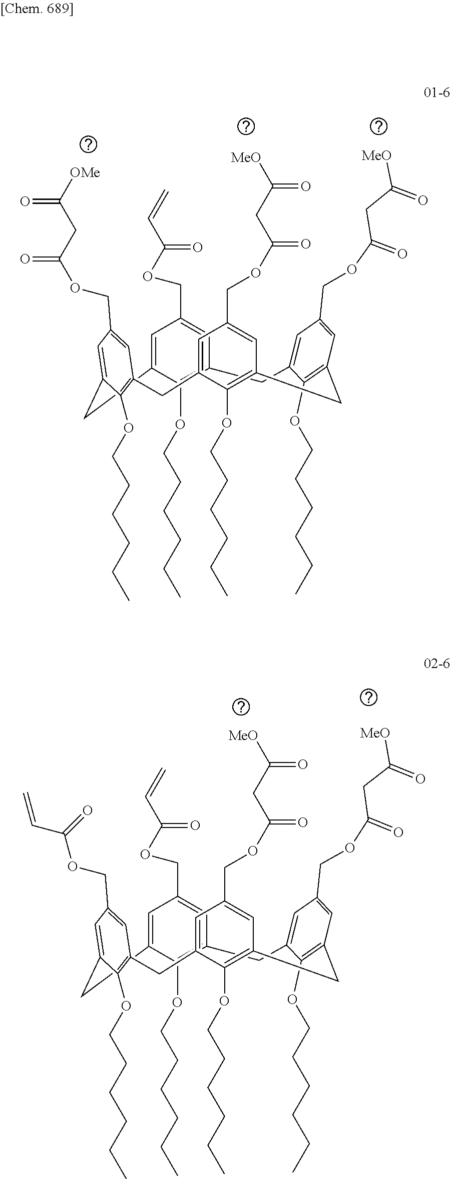 [SMILES: C=CC(=O)OCc1cc2c(OCCCCCC)c(c1)Cc1cc(COC(=O)CC(=O)OC)cc(c1OCCCCCC)Cc1cc(COC(=O)CC(=O)OC)cc(c1OCCCCCC)Cc1cc(COC(=O)CC(=O)OC)cc(c1OCCCCCC)C2 ; C=CC(=O)OCc1cc2c(OCCCCCC)c(c1)Cc1cc(COC(=O)CC(=O)OC)cc(c1OCCCCCC)Cc1cc(COC(=O)CC(=O)OC)cc(c1OCCCCCC)Cc1cc(COC(=O)C=C)cc(c1OCCCCCC)C2]

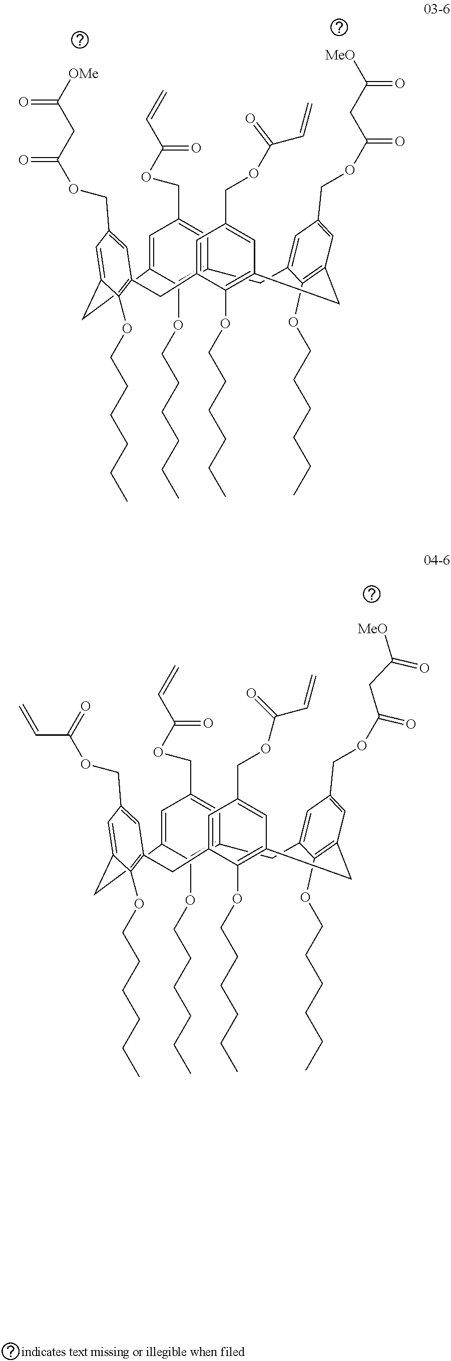 [SMILES: C=CC(=O)OCc1cc2c(OCCCCCC)c(c1)Cc1cc(COC(=O)CC(=O)OC)cc(c1OCCCCCC)Cc1cc(COC(=O)C=C)cc(c1OCCCCCC)Cc1cc(COC(=O)CC(=O)OC)cc(c1OCCCCCC)C2 ; C=CC(=O)OCc1cc2c(OCCCCCC)c(c1)Cc1cc(COC(=O)C=C)cc(c1OCCCCCC)Cc1cc(COC(=O)CC(=O)OC)cc(c1OCCCCCC)Cc1cc(COC(=O)C=C)cc(c1OCCCCCC)C2]

### Example 34

The same procedure as in Example 33 was repeated except that the compound obtained in Synthesis Example 50 (3.0 g, 4.99 mmol) was used instead of the compound obtained in Synthesis Example 49, to thereby obtain the target compounds 01-1, 02-1, 03-1, and 04-1 as follows. 01-1 (0.601 g, yield 12.6%). A mixture of 02-1 and 03-1 (2.429 g, yield 53.5%). 04-1 (0.616 g, yield 14.3%).

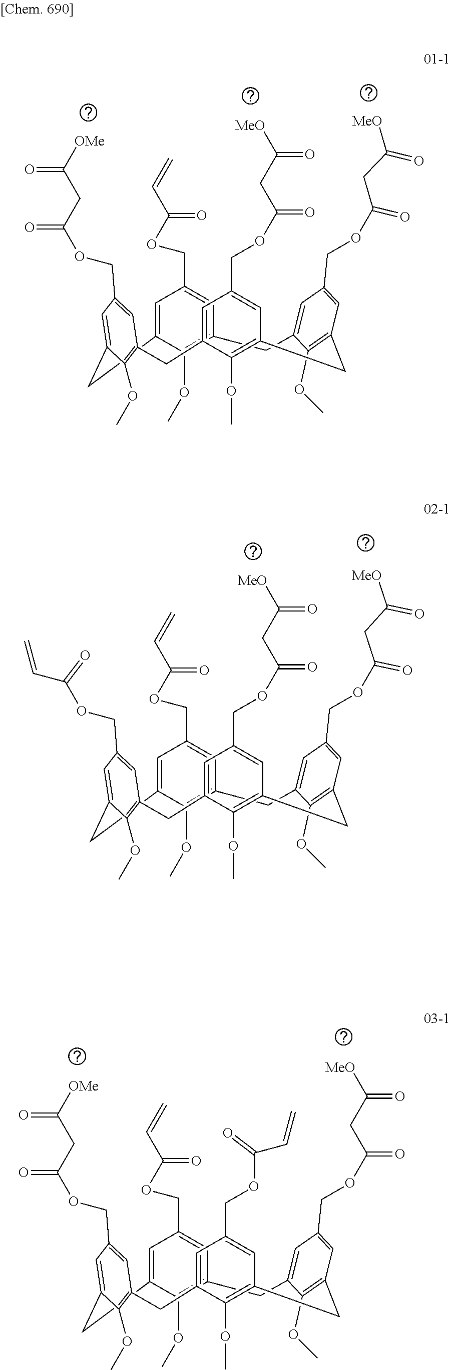 [SMILES: C=CC(=O)OCc1cc2c(OC)c(c1)Cc1cc(COC(=O)CC(=O)OC)cc(c1OC)Cc1cc(COC(=O)CC(=O)OC)cc(c1OC)Cc1cc(COC(=O)CC(=O)OC)cc(c1OC)C2 ; C=CC(=O)OCc1cc2c(OC)c(c1)Cc1cc(COC(=O)CC(=O)OC)cc(c1OC)Cc1cc(COC(=O)CC(=O)OC)cc(c1OC)Cc1cc(COC(=O)C=C)cc(c1OC)C2 ; C=CC(=O)OCc1cc2c(OC)c(c1)Cc1cc(COC(=O)CC(=O)OC)cc(c1OC)Cc1cc(COC(=O)C=C)cc(c1OC)Cc1cc(COC(=O)CC(=O)OC)cc(c1OC)C2]

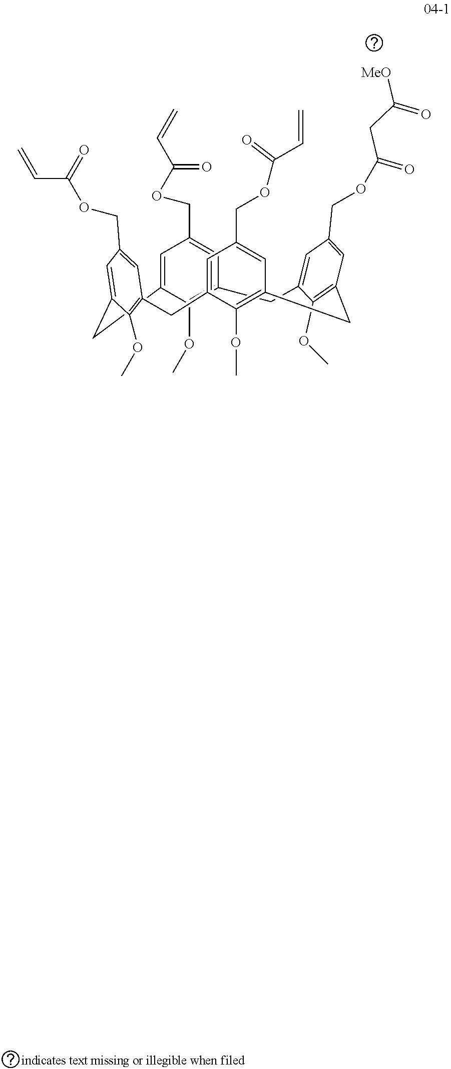 [SMILES: C=CC(=O)OCc1cc2c(OC)c(c1)Cc1cc(COC(=O)C=C)cc(c1OC)Cc1cc(COC(=O)CC(=O)OC)cc(c1OC)Cc1cc(COC(=O)C=C)cc(c1OC)C2]

### Example 35

The same procedure as in Example 33 was repeated except that the compound obtained in Synthesis Example 51 (3.0 g, 3.9 mmol) was used instead of the compound obtained in Synthesis Example 49, to thereby obtain the target compounds 01-4, 02-4, 03-4, and 04-4 as follows. 01-4 (0.640 g, yield 14.6%). A mixture of 02-4 and 03-4 (2.370 g, yield 56.4%). 04-4 (0.555 g, yield 13.8%).

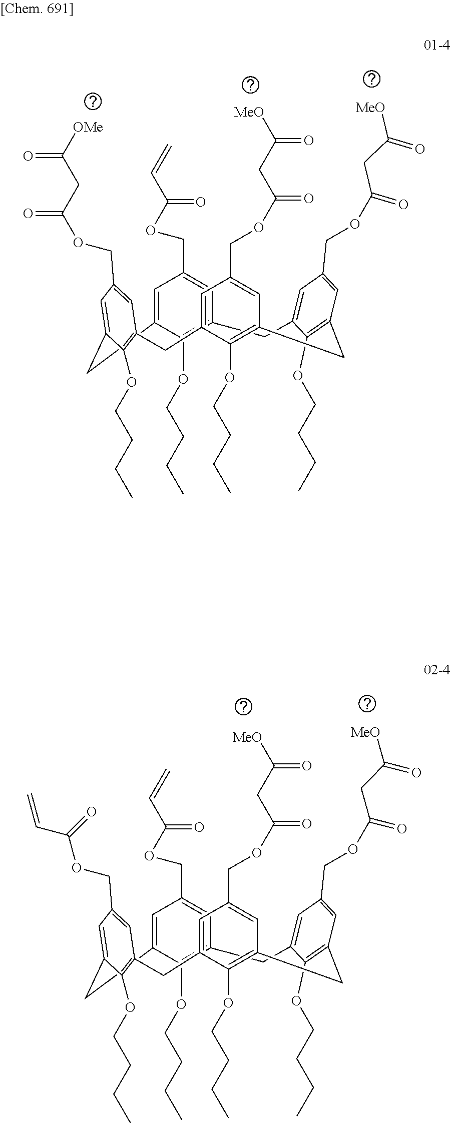 [SMILES: C=CC(=O)OCc1cc2c(OCCCC)c(c1)Cc1cc(COC(=O)CC(=O)OC)cc(c1OCCCC)Cc1cc(COC(=O)CC(=O)OC)cc(c1OCCCC)Cc1cc(COC(=O)CC(=O)OC)cc(c1OCCCC)C2 ; C=CC(=O)OCc1cc2c(OCCCC)c(c1)Cc1cc(COC(=O)CC(=O)OC)cc(c1OCCCC)Cc1cc(COC(=O)CC(=O)OC)cc(c1OCCCC)Cc1cc(COC(=O)C=C)cc(c1OCCCC)C2]

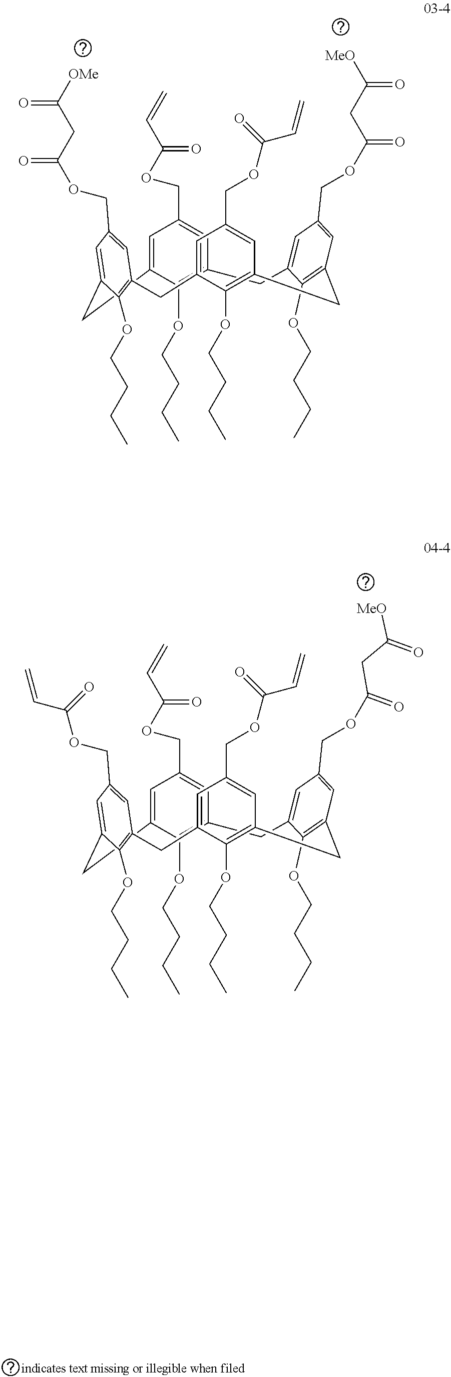 [SMILES: C=CC(=O)OCc1cc2c(OCCCC)c(c1)Cc1cc(COC(=O)CC(=O)OC)cc(c1OCCCC)Cc1cc(COC(=O)C=C)cc(c1OCCCC)Cc1cc(COC(=O)CC(=O)OC)cc(c1OCCCC)C2 ; C=CC(=O)OCc1cc2c(OCCCC)c(c1)Cc1cc(COC(=O)C=C)cc(c1OCCCC)Cc1cc(COC(=O)CC(=O)OC)cc(c1OCCCC)Cc1cc(COC(=O)C=C)cc(c1OCCCC)C2]

### Example 36

The same procedure as in Example 33 was repeated except that the compound obtained in Synthesis Example 52 (3.0 g, 3.2 mmol) was used instead of the compound obtained in Synthesis Example 49, to thereby obtain the target compounds 01-7, 02-7, 03-7, and 04-7 as follows. 01-7 (0.558 g, yield 13.5%). A mixture of 02-7 and 03-7 (2.292 g, yield 57.5%). 04-7 (0.484 g, yield 12.6%).

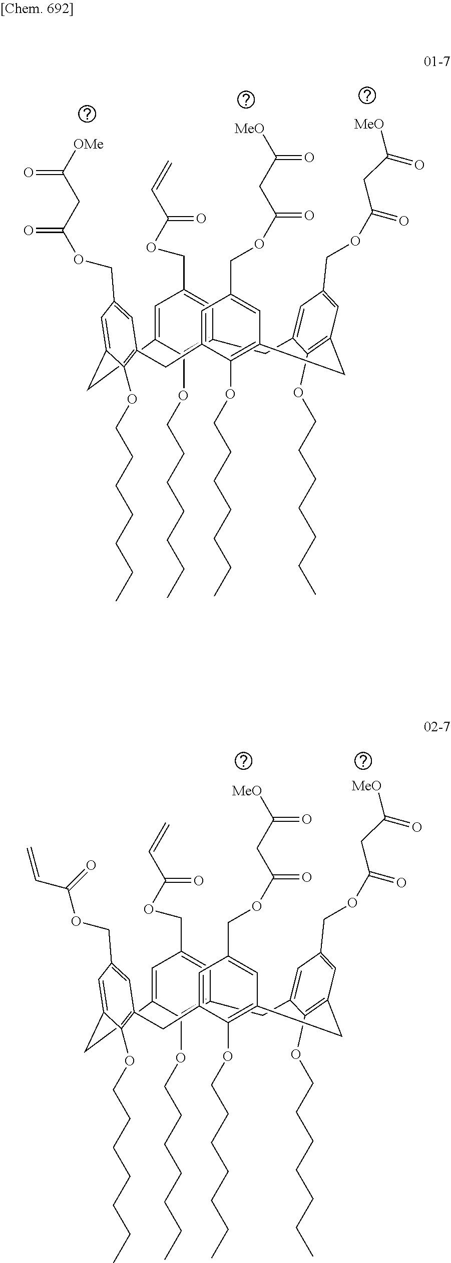 [SMILES: C=CC(=O)OCc1cc2c(OCCCCCCC)c(c1)Cc1cc(COC(=O)CC(=O)OC)cc(c1OCCCCCCC)Cc1cc(COC(=O)CC(=O)OC)cc(c1OCCCCCCC)Cc1cc(COC(=O)CC(=O)OC)cc(c1OCCCCCCC)C2 ; C=CC(=O)OCc1cc2c(OCCCCCCC)c(c1)Cc1cc(COC(=O)CC(=O)OC)cc(c1OCCCCCCC)Cc1cc(COC(=O)CC(=O)OC)cc(c1OCCCCCCC)Cc1cc(COC(=O)C=C)cc(c1OCCCCCCC)C2]

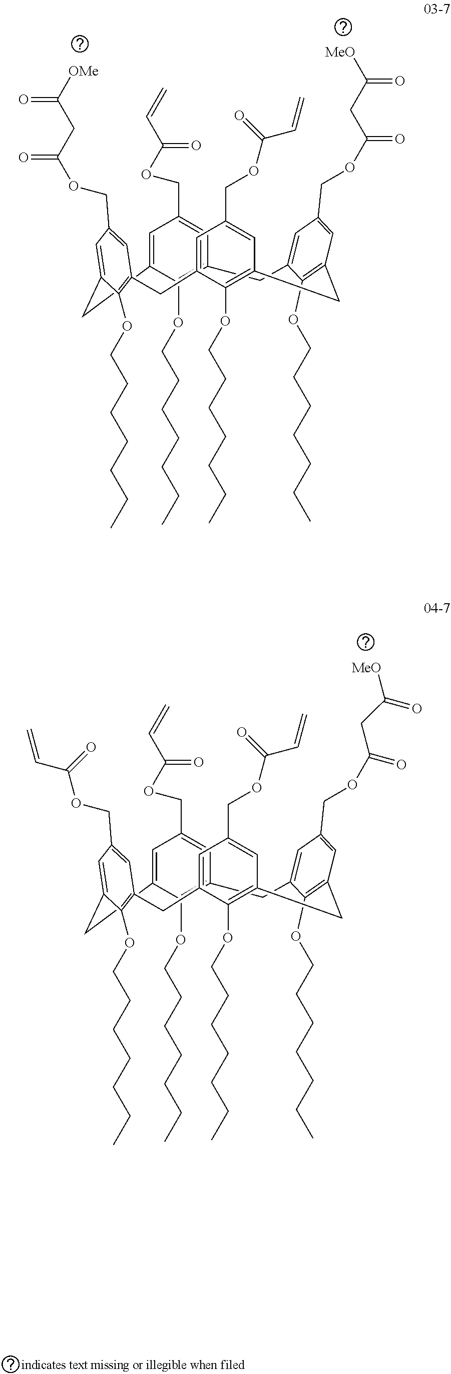 [SMILES: C=CC(=O)OCc1cc2c(OCCCCCCC)c(c1)Cc1cc(COC(=O)CC(=O)OC)cc(c1OCCCCCCC)Cc1cc(COC(=O)C=C)cc(c1OCCCCCCC)Cc1cc(COC(=O)CC(=O)OC)cc(c1OCCCCCCC)C2 ; C=CC(=O)OCc1cc2c(OCCCCCCC)c(c1)Cc1cc(COC(=O)C=C)cc(c1OCCCCCCC)Cc1cc(COC(=O)CC(=O)OC)cc(c1OCCCCCCC)Cc1cc(COC(=O)C=C)cc(c1OCCCCCCC)C2]

### Example 37

The same procedure as in Example 33 was repeated except that the compound obtained in Synthesis Example 53 (3.0 g, 1.93 mmol) was used instead of the compound obtained in Synthesis Example 49, to thereby obtain the target compounds 01-18, 02-18, 03-18, and 04-18 as follows. 01-18 (0.390 g, yield 10.6%). A mixture of 02-18 and 03-18 (1.934 g, yield 53.8%). 04-18 (0.617 g, yield 17.6%).

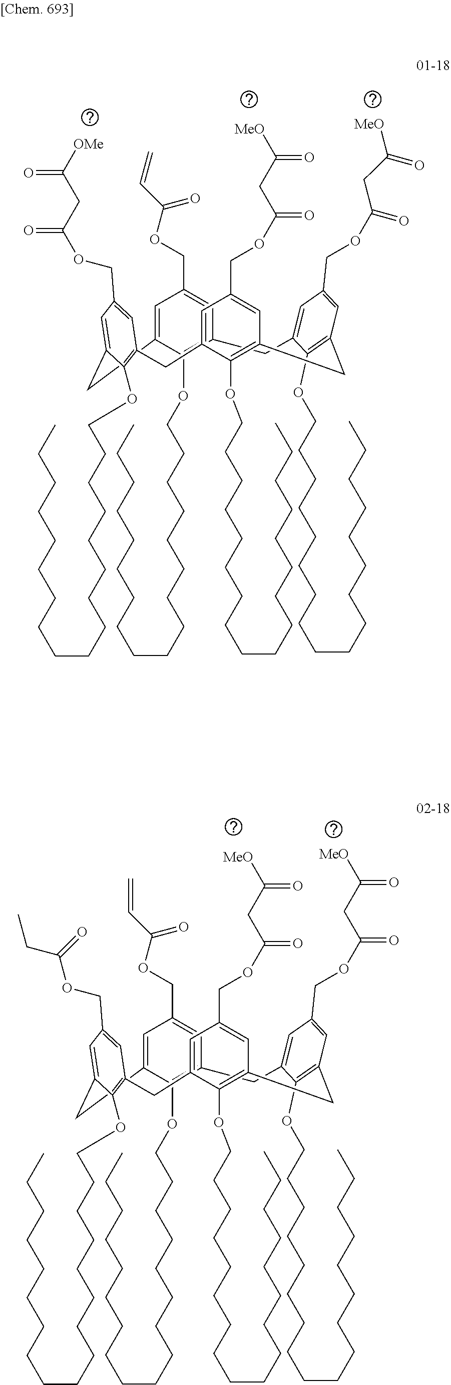 [SMILES: C=CC(=O)OCc1cc2c(OCCCCCCCCCCCCCCCCCC)c(c1)Cc1cc(COC(=O)CC(=O)OC)cc(c1OCCCCCCCCCCCCCCCCCC)Cc1cc(COC(=O)CC(=O)OC)cc(c1OCCCCCCCCCCCCCCCCCC)Cc1cc(COC(=O)CC(=O)OC)cc(c1OCCCCCCCCCCCCCCCCCC)C2 ; C=CC(=O)OCc1cc2c(OCCCCCCCCCCCCCCCCCC)c(c1)Cc1cc(COC(=O)CC(=O)OC)cc(c1OCCCCCCCCCCCCCCCCCC)Cc1cc(COC(=O)CC(=O)OC)cc(c1OCCCCCCCCCCCCCCCCCC)Cc1cc(COC(=O)CC)cc(c1OCCCCCCCCCCCCCCCCCC)C2]

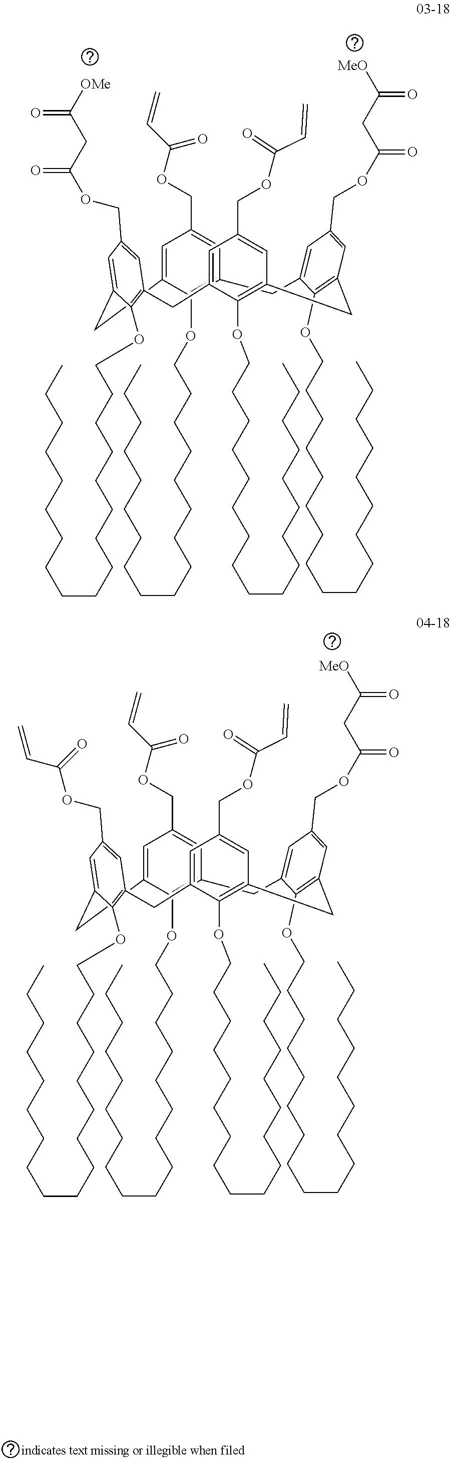 [SMILES: C=CC(=O)OCc1cc2c(OCCCCCCCCCCCCCCCCCC)c(c1)Cc1cc(COC(=O)CC(=O)OC)cc(c1OCCCCCCCCCCCCCCCCCC)Cc1cc(COC(=O)C=C)cc(c1OCCCCCCCCCCCCCCCCCC)Cc1cc(COC(=O)CC(=O)OC)cc(c1OCCCCCCCCCCCCCCCCCC)C2 ; C=CC(=O)OCc1cc2c(OCCCCCCCCCCCCCCCCCC)c(c1)Cc1cc(COC(=O)C=C)cc(c1OCCCCCCCCCCCCCCCCCC)Cc1cc(COC(=O)CC(=O)OC)cc(c1OCCCCCCCCCCCCCCCCCC)Cc1cc(COC(=O)C=C)cc(c1OCCCCCCCCCCCCCCCCCC)C2]

### Synthesis Example 64

A 100 mL four-necked flask equipped with a stirrer, a thermometer, and a reflux condenser tube was charged with 2.00 g (2.27 mmol) of the compound obtained in Synthesis Example 49, 3.57 g (13.62 mmol) of triphenylphosphine, 2.95 g (13.62 mmol) of 2-[[[(1,1-dimethylethyl)dimethylsilyl]oxy]2-propenoic acid, and 38 mL of tetrahydrofuran, and the mixture was stirred. Then 2.75 g (13.62 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 2.85 g, yield 75.0%).

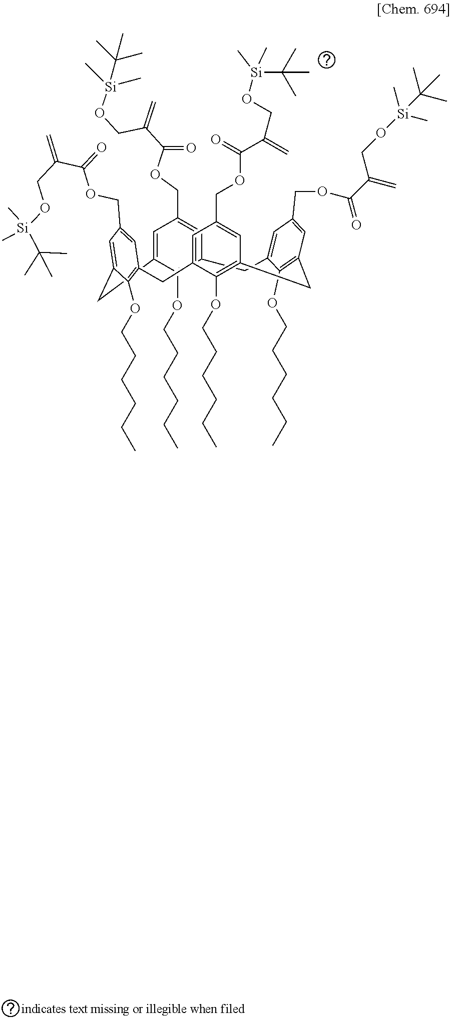 [SMILES: C=C(CO[Si](C)(C)C(C)(C)C)C(=O)OCc1cc2c(OCCCCCC)c(c1)Cc1cc(COC(=O)C(=C)CO[Si](C)(C)C(C)(C)C)cc(c1OCCCCCC)Cc1cc(COC(=O)C(=C)CO[Si](C)(C)C(C)(C)C)cc(c1OCCCCCC)Cc1cc(COC(=O)C(=C)CO[Si](C)(C)C(C)(C)C)cc(c1OCCCCCC)C2]

### Synthesis Example 65

The same procedure as in Synthesis Example 64 was repeated except that the compound obtained in Synthesis Example 50 (2.00 g, 3.33 mmol) was used instead of the compound obtained in Synthesis Example 49, to thereby obtain a compound represented by the following formula (3.26 g, yield 70.2%).

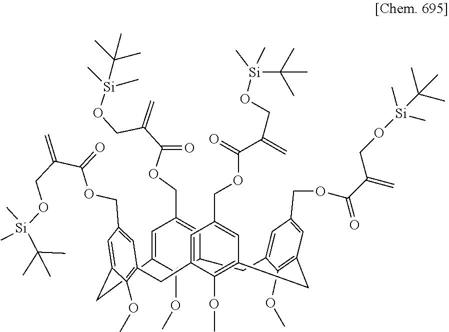 [SMILES: C=C(CO[Si](C)(C)C(C)(C)C)C(=O)OCc1cc2c(OC)c(c1)Cc1cc(COC(=O)C(=C)CO[Si](C)(C)C(C)(C)C)cc(c1OC)Cc1cc(COC(=O)C(=C)CO[Si](C)(C)C(C)(C)C)cc(c1OC)Cc1cc(COC(=O)C(=C)CO[Si](C)(C)C(C)(C)C)cc(c1OC)C2]

### Synthesis Example 66

The same procedure as in Synthesis Example 64 was repeated except that the compound obtained in Synthesis Example 51 (2.00 g, 2.60 mmol) was used instead of the compound obtained in Synthesis Example 49, to thereby obtain a compound represented by the following formula (3.12 g, yield 76.8%).

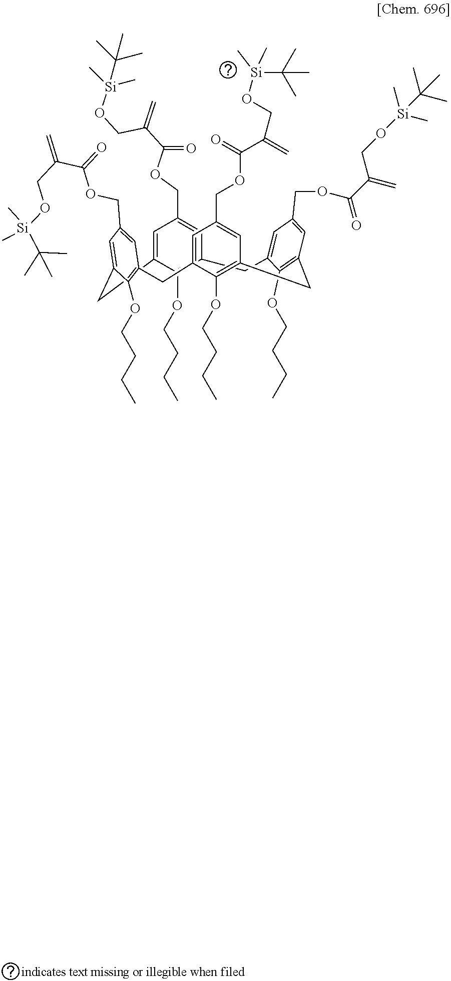 [SMILES: C=C(CO[Si](C)(C)C(C)(C)C)C(=O)OCc1cc2c(OCCCC)c(c1)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)Cc1cc(COC(=O)C(=C)CO[Si](C)(C)C(C)(C)C)cc(c1OCCCC)C2]

### Synthesis Example 67

The same procedure as in Synthesis Example 64 was repeated except that the compound obtained in Synthesis Example 52 (2.00 g, 2.13 mmol) was used instead of the compound obtained in Synthesis Example 49, to thereby obtain a compound represented by the following formula (2.74 g, yield 74.2%).

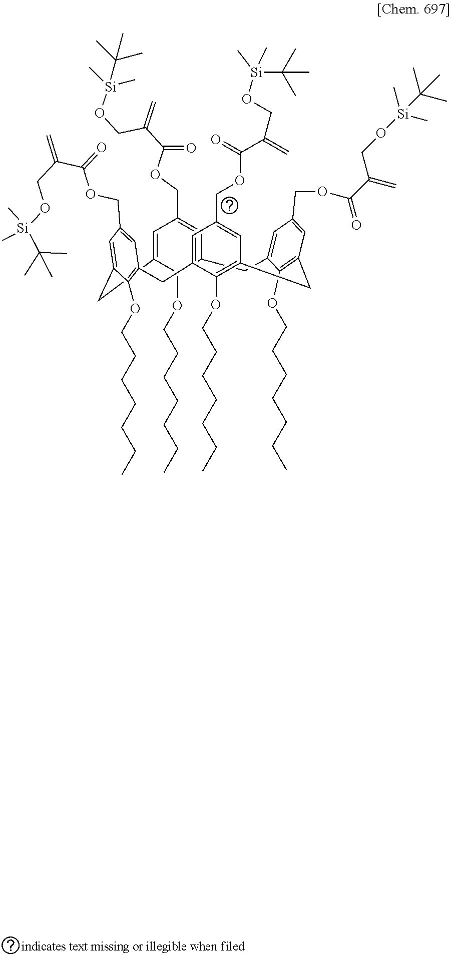 [SMILES: C=C(CO[Si](C)(C)C(C)(C)C)C(=O)OCc1cc2c(OCCCCCCC)c(c1)Cc1cc(COC(=O)C(=C)CO[Si](C)(C)C(C)(C)C)cc(c1OCCCCCCC)Cc1cc(COC(=O)C(=C)CO[Si](C)(C)C(C)(C)C)cc(c1OCCCCCCC)Cc1cc(COC(=O)C(=C)CO[Si](C)(C)C(C)(C)C)cc(c1OCCCCCCC)C2]

### Synthesis Example 68

The same procedure as in Synthesis Example 62 was repeated except that the compound obtained in Synthesis Example 53 (2.00 g, 1.29 mmol) was used instead of the compound obtained in Synthesis Example 49, to thereby obtain a compound represented by the following formula (2.58 g, yield 85.3%).

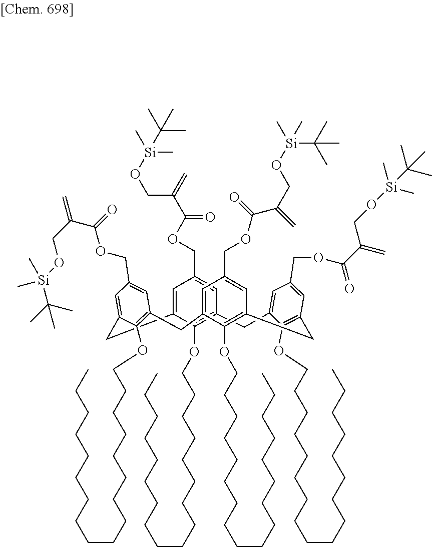 [SMILES: C=C(CO[Si](C)(C)C(C)(C)C)C(=O)OCc1cc2c(OCCCCCCCCCCCCCCCCCC)c(c1)Cc1cc(COC(=O)C(=C)CO[Si](C)(C)C(C)(C)C)cc(c1OCCCCCCCCCCCCCCCCCC)Cc1cc(COC(=O)C(=C)CO[Si](C)(C)C(C)(C)C)cc(c1OCCCCCCCCCCCCCCCCCC)Cc1cc(COC(=O)C(=C)CO[Si](C)(C)C(C)(C)C)cc(c1OCCCCCCCCCCCCCCCCCC)C2]

### Synthesis Example 69

A 100 mL four-necked flask equipped with a stirrer, a thermometer, and a reflux condenser tube was charged with 2.50 g (1.49 mmol) of the compound obtained in Synthesis Example 64, 0.538 g (8.96 mmol) of acetic acid, and 60 mL of tetrahydrofuran, and the mixture was stirred. A colorless transparent solution. Then tetrabutylammonium fluoride (an about 1 mol/L tetrahydrofuran solution 8.96 mL (8.96 mmol) was slowly added dropwise to the mixture placed in an ice bath under stirring, and the resulting mixture was further stirred at room temperature for 12 hours. A saturated aqueous ammonium chloride solution was added to the reaction mixture, and then 30 mL of chloroform was added. The reaction mixture was transferred to a separatory funnel, and the organic layer was separated. The aqueous layer was extracted with 30 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula as a white solid (yield amount 1.663 g, yield 91.5%).

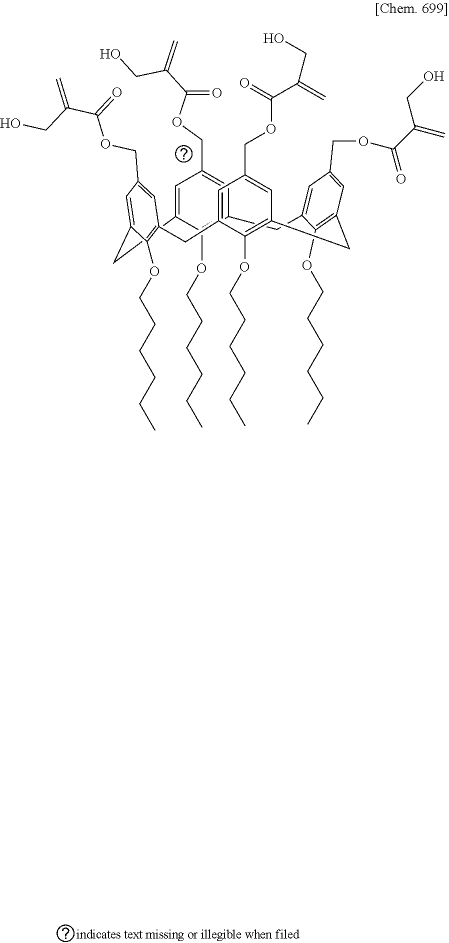 [SMILES: C=C(CO)C(=O)OCc1cc2c(OCCCCCC)c(c1)Cc1cc(COC(=O)C(=C)CO)cc(c1OCCCCCC)Cc1cc(COC(=O)C(=C)CO)cc(c1OCCCCCC)Cc1cc(COC(=O)C(=C)CO)cc(c1OCCCCCC)C2]

### Synthesis Example 70

The same procedure as in Synthesis Example 69 was repeated except that the compound obtained in Synthesis Example 65 (2.5 g, 1.79 mmol) was used instead of the compound obtained in Synthesis Example 64, to thereby obtain a compound represented by the following formula (1.551 g, yield 92.3%).

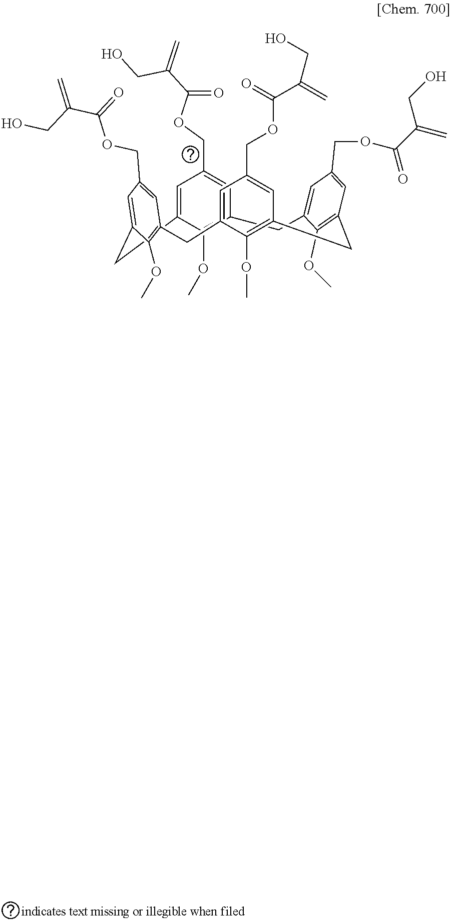 [SMILES: C=C(CO)C(=O)OCc1cc2c(OC)c(c1)Cc1cc(COC(=O)C(=C)CO)cc(c1OC)Cc1cc(COC(=O)C(=C)CO)cc(c1OC)Cc1cc(COC(=O)C(=C)CO)cc(c1OC)C2]

### Synthesis Example 71

The same procedure as in Synthesis Example 69 was repeated except that the compound obtained in Synthesis Example 66 (2.5 g, 1.60 mmol) was used instead of the compound obtained in Synthesis Example 64, to thereby obtain a compound represented by the following formula (1.671 g, yield 94.5%).

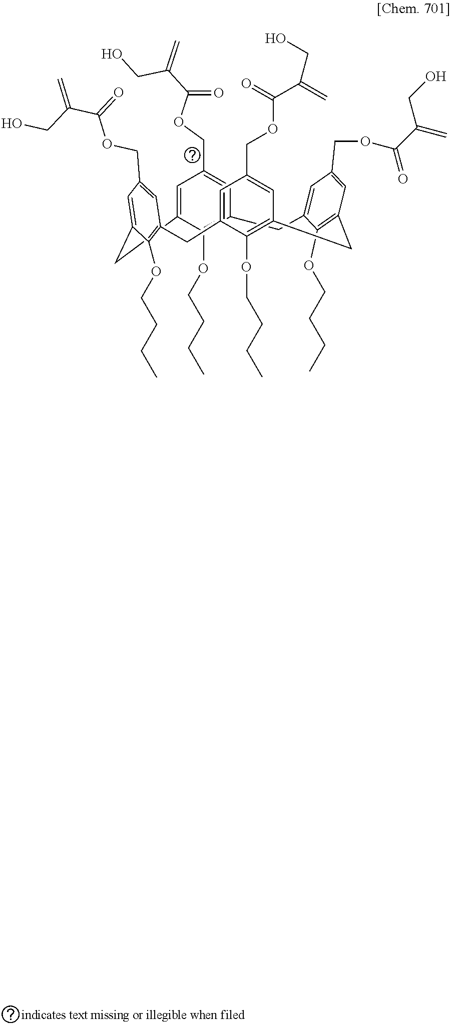 [SMILES: C=C(CO)C(=O)OCc1cc2c(OCCCC)c(c1)Cc1cc(COC(=O)C(=C)CO)cc(c1OCCCC)Cc1cc(COC(=O)C(=C)CO)cc(c1OCCCC)Cc1cc(COC(=O)C(=C)CO)cc(c1OCCCC)C2]

### Synthesis Example 72

The same procedure as in Synthesis Example 69 was repeated except that the compound obtained in Synthesis Example 67 (2.5 g, 1.44 mmol) was used instead of the compound obtained in Synthesis Example 64, to thereby obtain a compound represented by the following formula (1.759 g, yield 95.6%).

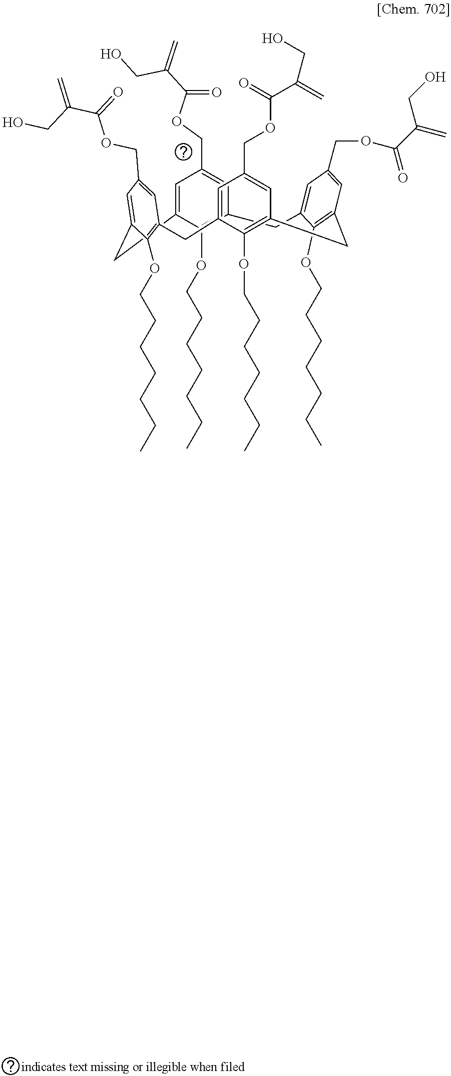 [SMILES: C=C(CO)C(=O)OCc1cc2c(OCCCCCCC)c(c1)Cc1cc(COC(=O)C(=C)CO)cc(c1OCCCCCCC)Cc1cc(COC(=O)C(=C)CO)cc(c1OCCCCCCC)Cc1cc(COC(=O)C(=C)CO)cc(c1OCCCCCCC)C2]

### Synthesis Example 73

The same procedure as in Synthesis Example 69 was repeated except that the compound obtained in Synthesis Example 68 (2.50 g, 1.06 mmol) was used instead of the compound obtained in Synthesis Example 64, to thereby obtain a compound represented by the following formula (1.90 g, yield 94.8%).

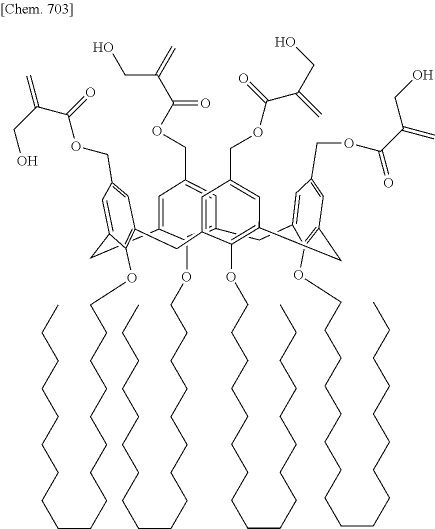 [SMILES: C=C(CO)C(=O)OCc1cc2c(OCCCCCCCCCCCCCCCCCC)c(c1)Cc1cc(COC(=O)C(=C)CO)cc(c1OCCCCCCCCCCCCCCCCCC)Cc1cc(COC(=O)C(=C)CO)cc(c1OCCCCCCCCCCCCCCCCCC)Cc1cc(COC(=O)C(=C)CO)cc(c1OCCCCCCCCCCCCCCCCCC)C2]

### Example 38

A 100 mL four-necked flask equipped with a stirrer, a dropping funnel, and a thermometer was charged, in a nitrogen atmosphere, with the compound obtained in Synthesis Example 69 (1.5 g, 1.23 mmol), triethylamine (0.997 g, 9.86 mmol), and methylene chloride (15 mL), and the mixture was stirred under cooling with ice. A solution prepared by dissolving methyl malonyl chloride (1.009 g, 7.39 mmol) in methylene chloride (3 mL) was slowly added dropwise. After completion of the dropwise addition, the resulting mixture was stirred at room temperature for 8 hours. Water was added to the reaction mixture, and the resulting mixture was extracted with chloroform (40 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator, and the obtained yellow viscous liquid was purified by silica gel column chromatography to thereby obtain the target compound 05-6 (yield amount 1.738 g, yield 87.2%).

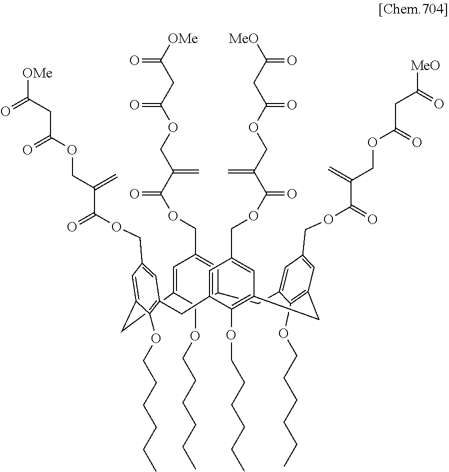 [SMILES: C=C(COC(=O)CC(=O)OC)C(=O)OCc1cc2c(OCCCCCC)c(c1)Cc1cc(COC(=O)C(=C)COC(=O)CC(=O)OC)cc(c1OCCCCCC)Cc1cc(COC(=O)C(=C)COC(=O)CC(=O)OC)cc(c1OCCCCCC)Cc1cc(COC(=O)C(=C)COC(=O)CC(=O)OC)cc(c1OCCCCCC)C2]

### Example 39

The same procedure as in Example 38 was repeated except that the compound obtained in Synthesis Example 70 (1.50 g, 1.60 mmol) was used instead of the compound obtained in Synthesis Example 69, to thereby obtain the target compound 05-1 (1.805 g, yield 84.3%).

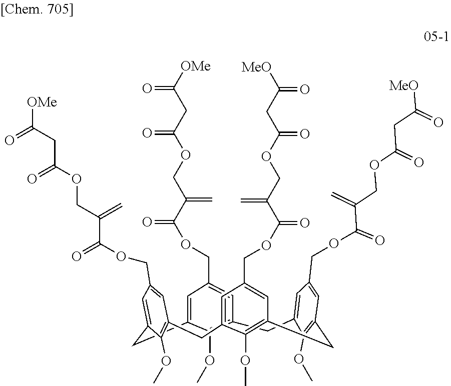 [SMILES: C=C(COC(=O)CC(=O)OC)C(=O)OCc1cc2c(OC)c(c1)Cc1cc(COC(=O)C(=C)COC(=O)CC(=O)OC)cc(c1OC)Cc1cc(COC(=O)C(=C)COC(=O)CC(=O)OC)cc(c1OC)Cc1cc(COC(=O)C(=C)COC(=O)CC(=O)OC)cc(c1OC)C2]

### Example 40

The same procedure as in Example 38 was repeated except that the compound obtained in Synthesis Example 71 (1.50 g, 1.36 mmol) was used instead of the compound obtained in Synthesis Example 69, to thereby obtain the target compound 05-4 (1.808 g, yield 88.5%).

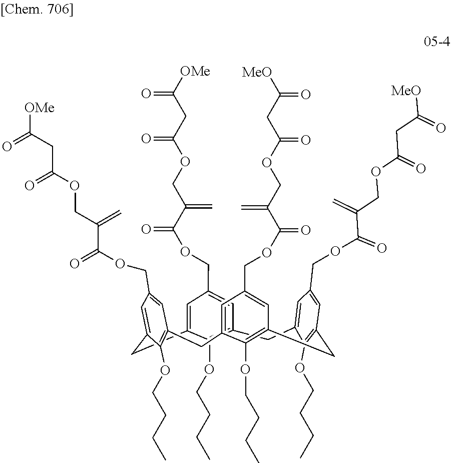 [SMILES: C=C(COC(=O)CC(=O)OC)C(=O)OCc1cc2c(OCCCC)c(c1)Cc1cc(COC(=O)C(=C)COC(=O)CC(=O)OC)cc(c1OCCCC)Cc1cc(COC(=O)C(=C)COC(=O)CC(=O)OC)cc(c1OCCCC)Cc1cc(COC(=O)C(=C)COC(=O)CC(=O)OC)cc(c1OCCCC)C2]

### Example 41

The same procedure as in Example 38 was repeated except that the compound obtained in Synthesis Example 72 (1.50 g, 1.18 mmol) was used instead of the compound obtained in Synthesis Example 69, to thereby obtain the target compound 05-7 (1.790 g, yield 90.8%).

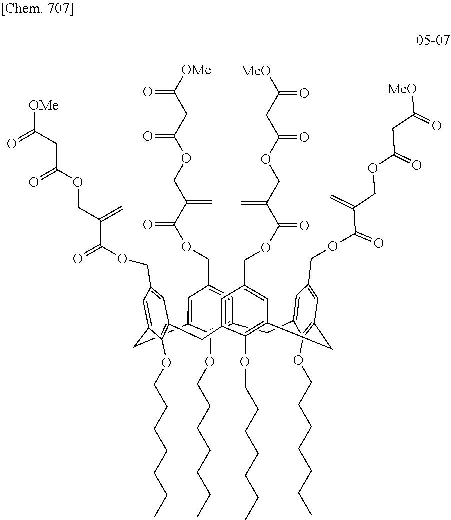 [SMILES: C=C(COC(=O)CC(=O)OC)C(=O)OCc1cc2c(OCCCCCCC)c(c1)Cc1cc(COC(=O)C(=C)COC(=O)CC(=O)OC)cc(c1OCCCCCCC)Cc1cc(COC(=O)C(=C)COC(=O)CC(=O)OC)cc(c1OCCCCCCC)Cc1cc(COC(=O)C(=C)COC(=O)CC(=O)OC)cc(c1OCCCCCCC)C2]

### Example 42

The same procedure as in Example 38 was repeated except that the compound obtained in Synthesis Example 73 (1.5 g, 0.79 mmol) was used instead of the compound obtained in Synthesis Example 69, to thereby obtain the target compound 05-18 (1.592 g, yield 87.6%).

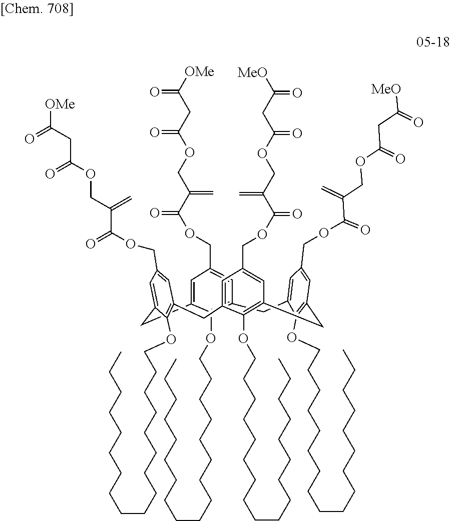 [SMILES: C=C(COC(=O)CC(=O)OC)C(=O)OCc1cc2c(OCCCCCCCCCCCCCCCCCC)c(c1)Cc1cc(COC(=O)C(=C)COC(=O)CC(=O)OC)cc(c1OCCCCCCCCCCCCCCCCCC)Cc1cc(COC(=O)C(=C)COC(=O)CC(=O)OC)cc(c1OCCCCCCCCCCCCCCCCCC)Cc1cc(COC(=O)C(=C)COC(=O)CC(=O)OC)cc(c1OCCCCCCCCCCCCCCCCCC)C2]

### Example 43

A 100 mL four-necked flask equipped with a stirrer, a dropping funnel, and a thermometer was charged, in a nitrogen atmosphere, with the compound obtained in Synthesis Example 59 (3.0 g, 3.02 mmol), triethylamine (2.445 g, 24.16 mmol), and methylene chloride (30.2 mL), and the mixture was stirred under cooling with ice. A solution prepared by dissolving acrylic acid chloride (0.656 g, 7.25 mmol) and methyl malonyl chloride (0.989 g, 7.25 mmol) in methylene chloride (5 mL) was slowly added dropwise. After completion of the dropwise addition, the resulting mixture was stirred at room temperature for 8 hours. Water was added to the reaction mixture, the resulting mixture was extracted with chloroform (50 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator to thereby obtain a yellow liquid. The yellow liquid was purified by silica gel column chromatography to thereby obtain the target compounds 06-6, 07-6, 08-6, and 09-6 as follows. 06-6 (0.501 g, yield 12.3%). A mixture of 07-6 and 08-6 (2.056 g, yield 52.3%). 09-6 (0.592 g, yield 15.6%).

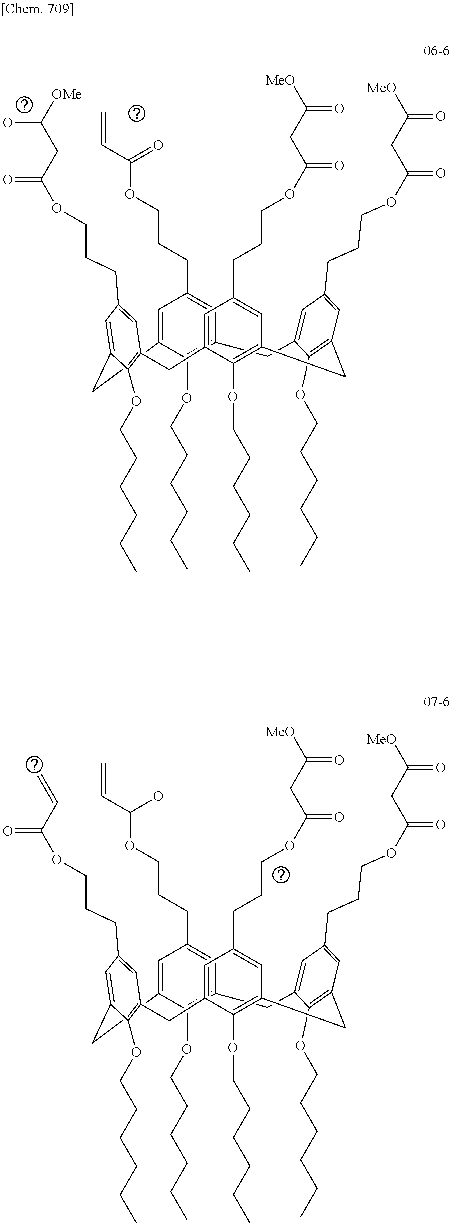 [SMILES: C=CC(=O)OCCCc1cc2c(OCCCCCC)c(c1)Cc1cc(CCCOC(=O)CC(O)OC)cc(c1OCCCCCC)Cc1cc(CCCOC(=O)CC(=O)OC)cc(c1OCCCCCC)Cc1cc(CCCOC(=O)CC(=O)OC)cc(c1OCCCCCC)C2 ; C=CC(=O)OCCCc1cc2c(OCCCCCC)c(c1)Cc1cc(CCCOC(O)C=C)cc(c1OCCCCCC)Cc1cc(CCCOC(=O)CC(=O)OC)cc(c1OCCCCCC)Cc1cc(CCCOC(=O)CC(=O)OC)cc(c1OCCCCCC)C2]

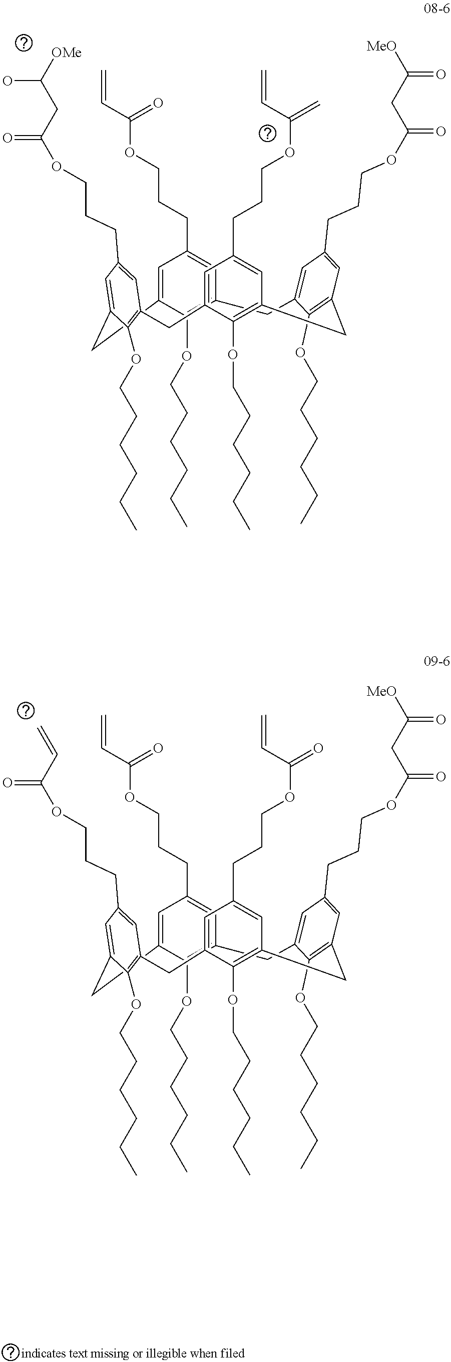 [SMILES: C=CC(=C)OCCCc1cc2c(OCCCCCC)c(c1)Cc1cc(CCCOC(=O)CC(O)OC)cc(c1OCCCCCC)Cc1cc(CCCOC(=O)C=C)cc(c1OCCCCCC)Cc1cc(CCCOC(=O)CC(=O)OC)cc(c1OCCCCCC)C2 ; C=CC(=O)OCCCc1cc2c(OCCCCCC)c(c1)Cc1cc(CCCOC(=O)C=C)cc(c1OCCCCCC)Cc1cc(CCCOC(=O)CC(=O)OC)cc(c1OCCCCCC)Cc1cc(CCCOC(=O)C=C)cc(c1OCCCCCC)C2]

### Example 44

The same procedure as in Example 43 was repeated except that the compound obtained in Synthesis Example 60 (3.00 g, 4.21 mmol) was used instead of the compound obtained in Synthesis Example 59, to thereby obtain the target compounds 06-1, 07-1, 08-1, and 09-1 as follows. 06-1 (0.530 g, yield 11.8%). A mixture of 07-1 and 08-1 (2.342 g, yield 54.5%). 09-1 (0.550 g, yield 13.4%).

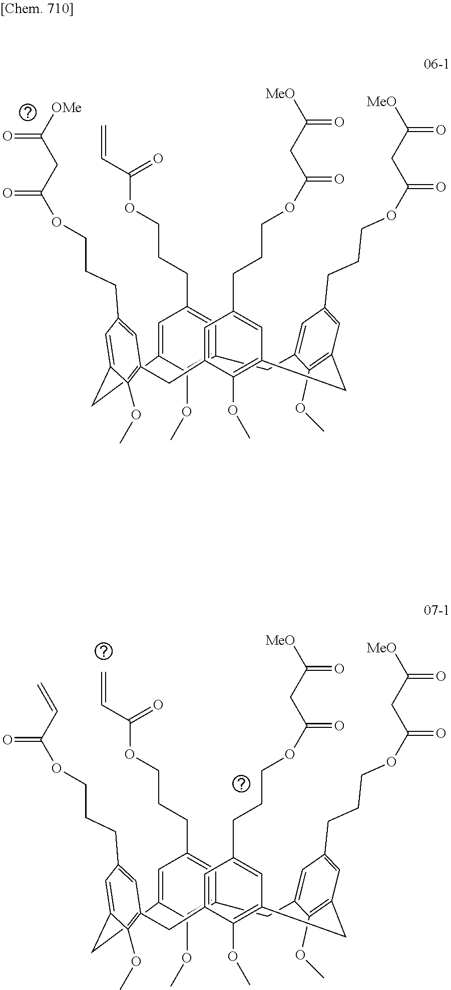 [SMILES: C=CC(=O)OCCCc1cc2c(OC)c(c1)Cc1cc(CCCOC(=O)CC(=O)OC)cc(c1OC)Cc1cc(CCCOC(=O)CC(=O)OC)cc(c1OC)Cc1cc(CCCOC(=O)CC(=O)OC)cc(c1OC)C2 ; C=CC(=O)OCCCc1cc2c(OC)c(c1)Cc1cc(CCCOC(=O)CC(=O)OC)cc(c1OC)Cc1cc(CCCOC(=O)CC(=O)OC)cc(c1OC)Cc1cc(CCCOC(=O)C=C)cc(c1OC)C2]

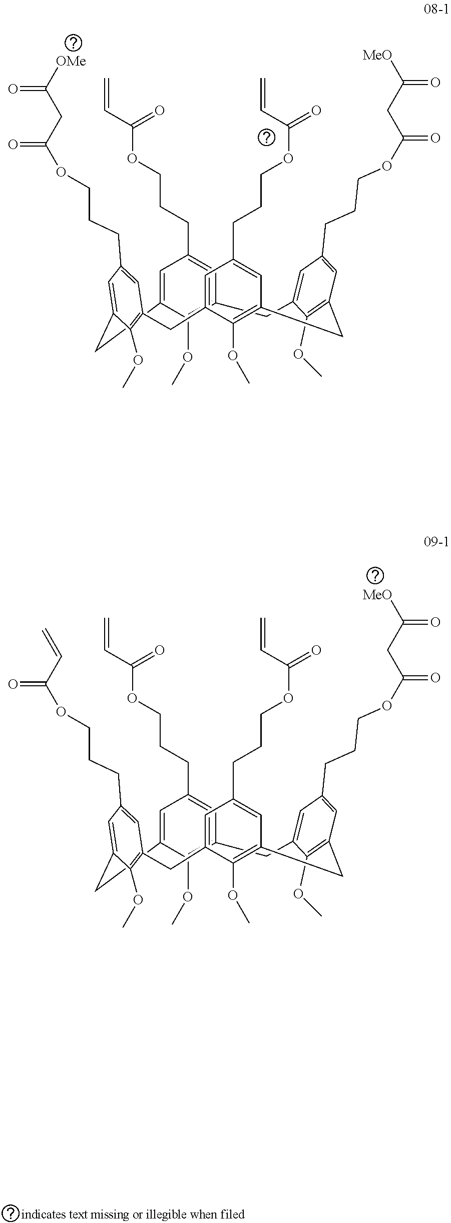 [SMILES: C=CC(=O)OCCCc1cc2c(OC)c(c1)Cc1cc(CCCOC(=O)CC(=O)OC)cc(c1OC)Cc1cc(CCCOC(=O)C=C)cc(c1OC)Cc1cc(CCCOC(=O)CC(=O)OC)cc(c1OC)C2 ; C=CC(=O)OCCCc1cc2c(OC)c(c1)Cc1cc(CCCOC(=O)C=C)cc(c1OC)Cc1cc(CCCOC(=O)CC(=O)OC)cc(c1OC)Cc1cc(CCCOC(=O)C=C)cc(c1OC)C2]

### Example 45

The same procedure as in Example 43 was repeated except that the compound obtained in Synthesis Example 61 (3.00 g, 3.40 mmol) was used instead of the compound obtained in Synthesis Example 59, to thereby obtain the target compounds 06-4, 07-4, 08-4, and 09-4 as follows. 06-4 (0.580 g, yield 13.8%). A mixture of 07-1 and 08-1 (2.211 g, yield 54.6%). 09-4 (0.564 g, yield 14.5%).

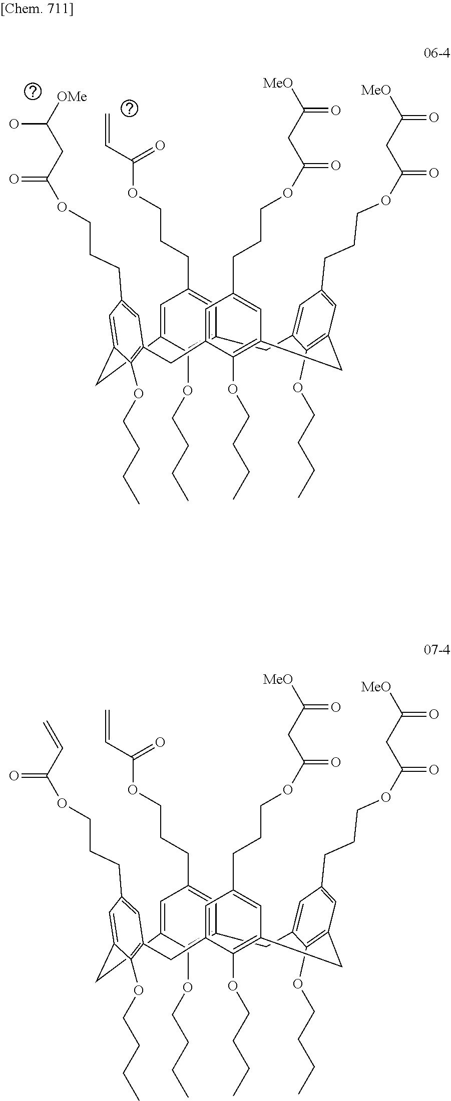 [SMILES: C=CC(=O)OCCCc1cc2c(OCCCC)c(c1)Cc1cc(CCCOC(=O)CC(O)OC)cc(c1OCCCC)Cc1cc(CCCOC(=O)CC(=O)OC)cc(c1OCCCC)Cc1cc(CCCOC(=O)CC(=O)OC)cc(c1OCCCC)C2 ; C=CC(=O)OCCCc1cc2c(OCCCC)c(c1)Cc1cc(CCCOC(=O)CC(=O)OC)cc(c1OCCCC)Cc1cc(CCCOC(=O)CC(=O)OC)cc(c1OCCCC)Cc1cc(CCCOC(=O)C=C)cc(c1OCCCC)C2]

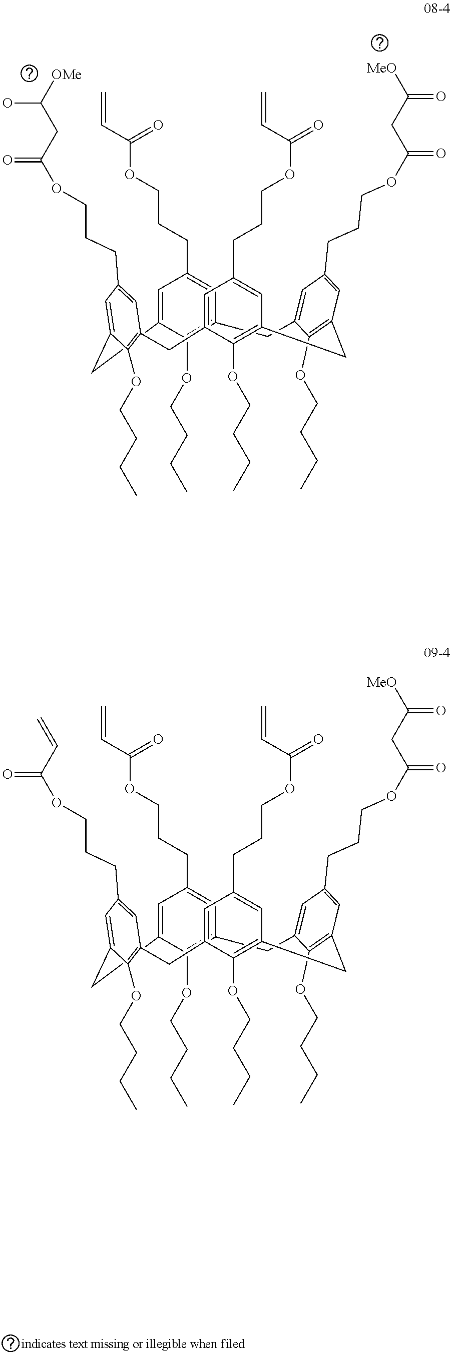 [SMILES: C=CC(=O)OCCCc1cc2c(OCCCC)c(c1)Cc1cc(CCCOC(=O)CC(O)OC)cc(c1OCCCC)Cc1cc(CCCOC(=O)C=C)cc(c1OCCCC)Cc1cc(CCCOC(=O)CC(=O)OC)cc(c1OCCCC)C2 ; C=CC(=O)OCCCc1cc2c(OCCCC)c(c1)Cc1cc(CCCOC(=O)C=C)cc(c1OCCCC)Cc1cc(CCCOC(=O)CC(=O)OC)cc(c1OCCCC)Cc1cc(CCCOC(=O)C=C)cc(c1OCCCC)C2]

### Example 46

The same procedure as in Example 43 was repeated except that the compound obtained in Synthesis Example 62 (3.00 g, 2.86 mmol) was used instead of the compound obtained in Synthesis Example 59, to thereby obtain the target compounds 06-7, 07-7, 08-7, and 09-7 as follows. 06-7 (0.510 g, yield 12.7%). A mixture of 07-7 and 08-7 (2.158 g, yield 55.6%). 09-7 (0.502 g, yield 13.4%).

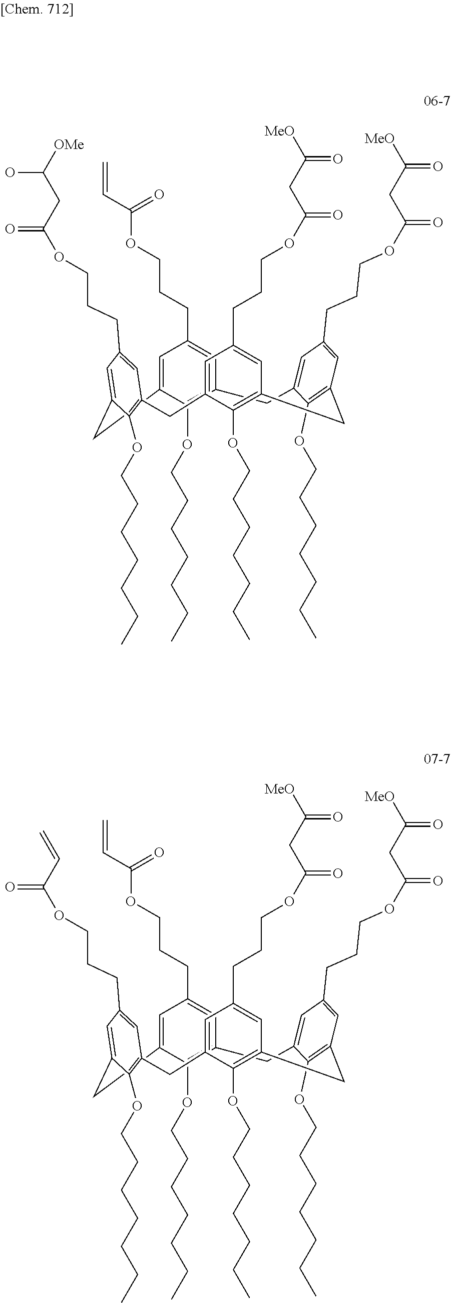 [SMILES: C=CC(=O)OCCCc1cc2c(OCCCCCCC)c(c1)Cc1cc(CCCOC(=O)CC(O)OC)cc(c1OCCCCCCC)Cc1cc(CCCOC(=O)CC(=O)OC)cc(c1OCCCCCCC)Cc1cc(CCCOC(=O)CC(=O)OC)cc(c1OCCCCCCC)C2 ; C=CC(=O)OCCCc1cc2c(OCCCCCCC)c(c1)Cc1cc(CCCOC(=O)CC(=O)OC)cc(c1OCCCCCCC)Cc1cc(CCCOC(=O)CC(=O)OC)cc(c1OCCCCCCC)Cc1cc(CCCOC(=O)C=C)cc(c1OCCCCCCC)C2]

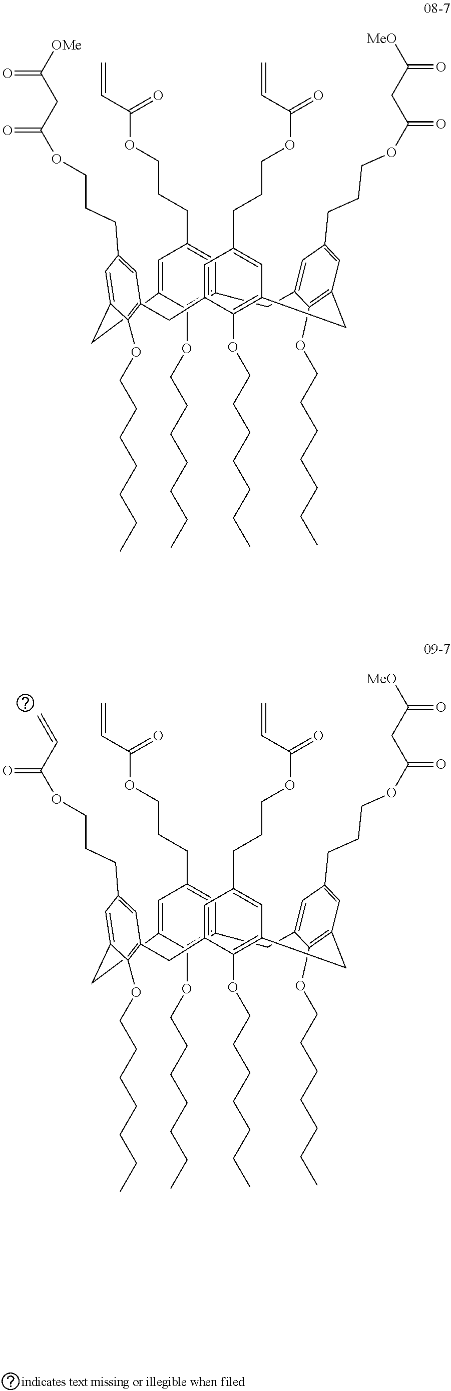 [SMILES: C=CC(=O)OCCCc1cc2c(OCCCCCCC)c(c1)Cc1cc(CCCOC(=O)CC(=O)OC)cc(c1OCCCCCCC)Cc1cc(CCCOC(=O)C=C)cc(c1OCCCCCCC)Cc1cc(CCCOC(=O)CC(=O)OC)cc(c1OCCCCCCC)C2 ; C=CC(=O)OCCCc1cc2c(OCCCCCCC)c(c1)Cc1cc(CCCOC(=O)C=C)cc(c1OCCCCCCC)Cc1cc(CCCOC(=O)CC(=O)OC)cc(c1OCCCCCCC)Cc1cc(CCCOC(=O)C=C)cc(c1OCCCCCCC)C2]

### Example 47

The same procedure as in Example 43 was repeated except that the compound obtained in Synthesis Example 63 (3.00 g, 1.80 mmol) was used instead of the compound obtained in Synthesis Example 59, to thereby obtain the target compounds 06-18, 07-18, 08-18, and 09-18 as follows. 06-18 (0.364 g, yield 10.3%). A mixture of 07-18 and 08-18 (1.187 g, yield 52.6%). 09-18 (0.566 g, yield 16.8%).

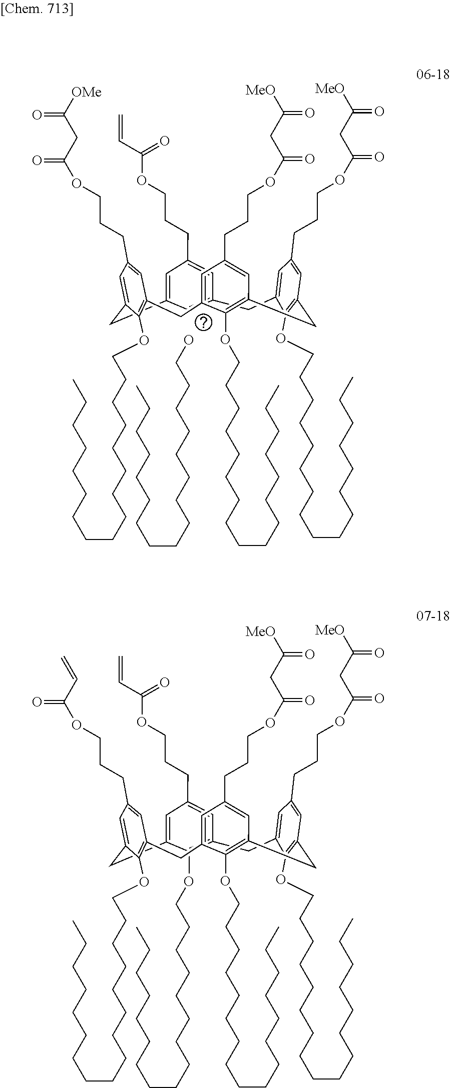

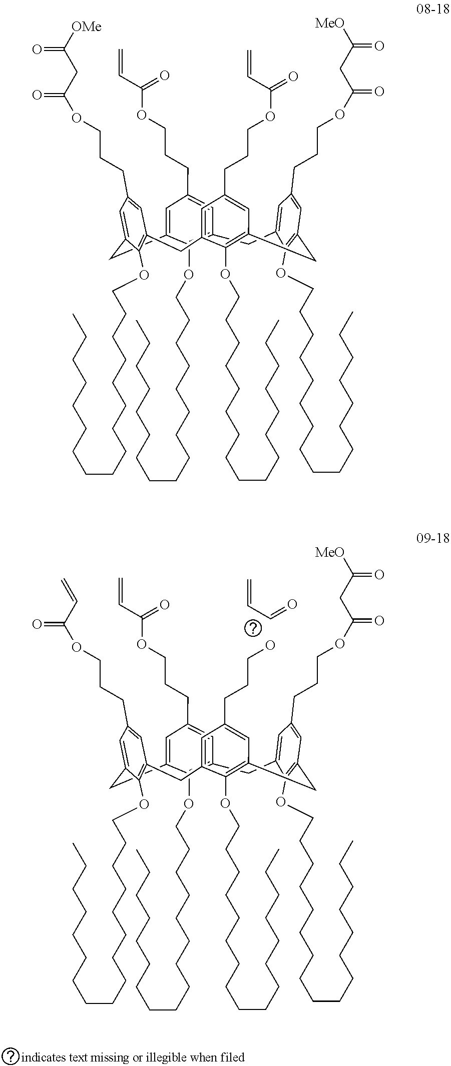

### Synthesis Example 74

A 100 mL four-necked flask equipped with a stirrer, a thermometer, and a reflux condenser tube was charged with 2.50 g (2.52 mmol) of the compound obtained in Synthesis Example 59, 3.96 g (15.10 mmol) of triphenylphosphine, 3.267 g (15.10 mmol) of 2-[[[(1,1-dimethylethyl)dimethylsilyll]oxy]2-propenoic acid, and 43 mL of tetrahydrofuran, and the mixture was stirred. Then 3.053 g (15.10 mmol) of diisopropyl azodicarboxylate was added dropwise to the mixture placed in an ice bath over 30 minutes, and the resulting mixture was further stirred at room temperature for 12 hours. The reaction solution was concentrated in an evaporator, and hexane was added to precipitate and remove by-products such as triphenylphosphine. The obtained yellow viscous liquid was purified by silica gel column chromatography to thereby obtain a compound represented by the following formula as a light yellow solid (yield amount 3.251 g, yield 72.3%).

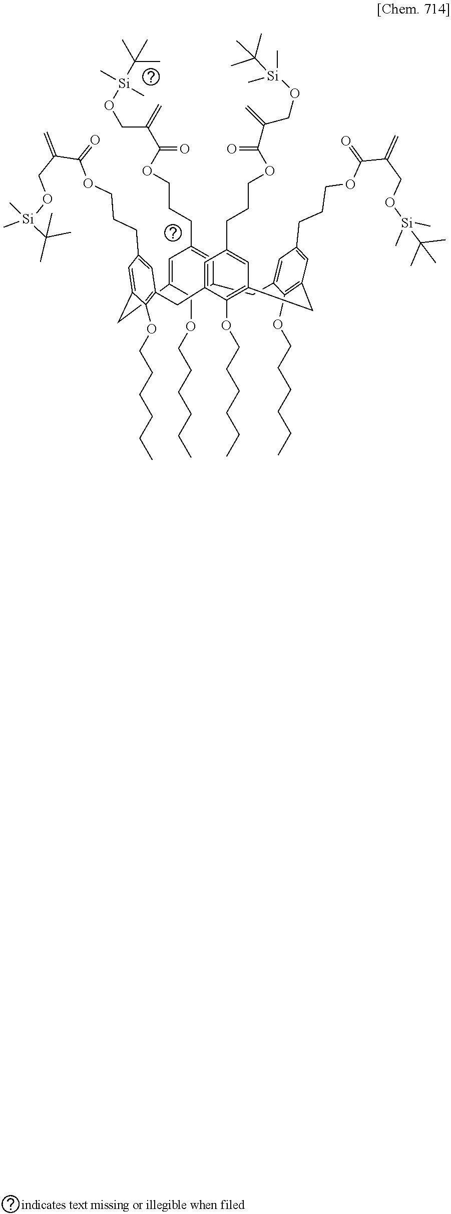 [SMILES: C=C(CO[Si](C)(C)C(C)(C)C)C(=O)OCCCc1cc2c(OCCCCCC)c(c1)Cc1cc(CCCOC(=O)C(=C)CO[Si](C)(C)C(C)(C)C)cc(c1OCCCCCC)Cc1cc(CCCOC(=O)C(=C)CO[Si](C)(C)C(C)(C)C)cc(c1OCCCCCC)Cc1cc(CCCOC(=O)C(=C)CO[Si](C)(C)C(C)(C)C)cc(c1OCCCCCC)C2]

### Synthesis Example 75

The same procedure as in Synthesis Example 74 was repeated except that the compound obtained in Synthesis Example 60 (2.50 g, 3.33 mmol) was used instead of the compound obtained in Synthesis Example 59, to thereby obtain a compound represented by the following formula (3.782 g, yield 71.6%).

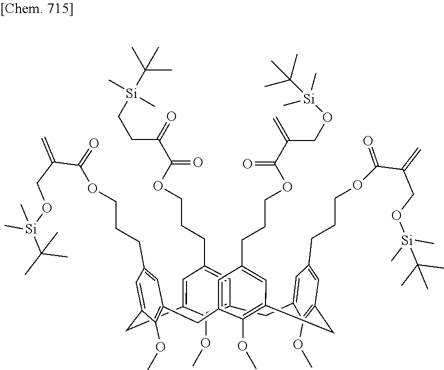 [SMILES: C=C(CO[Si](C)(C)C(C)(C)C)C(=O)OCCCc1cc2c(OC)c(c1)Cc1cc(CCCOC(=O)C(=C)CO[Si](C)(C)C(C)(C)C)cc(c1OC)Cc1cc(CCCOC(=O)C(=O)CC[Si](C)(C)C(C)(C)C)cc(c1OC)Cc1cc(CCCOC(=O)C(=C)CO[Si](C)(C)C(C)(C)C)cc(c1OC)C2]

### Synthesis Example 76

The same procedure as in Synthesis Example 74 was repeated except that the compound obtained in Synthesis Example 61 (2.50 g, 2.84 mmol) was used instead of the compound obtained in Synthesis Example 59, to thereby obtain a compound represented by the following formula (3.553 g, yield 74.8%).

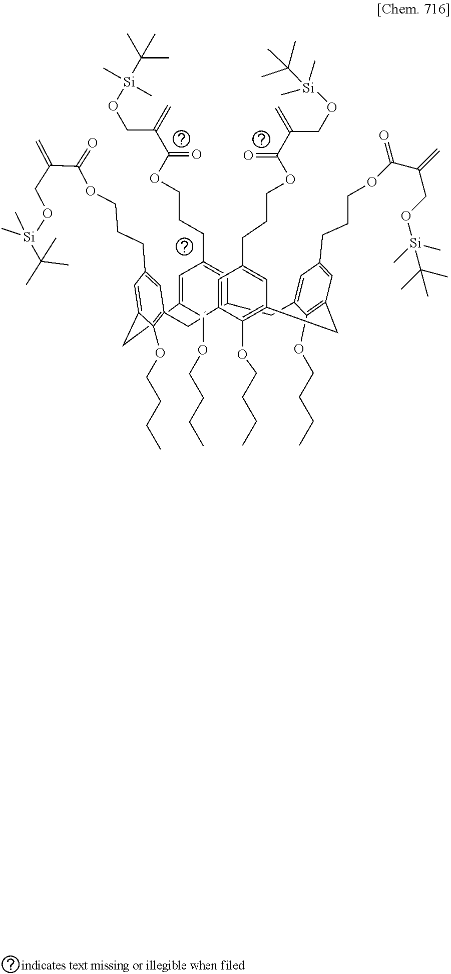 [SMILES: C=C(CO[Si](C)(C)C(C)(C)C)C(=O)OCCCc1cc2c(OCCCC)c(c1)Cc1cc(CCCOC(=O)C(=C)CO[Si](C)(C)C(C)(C)C)cc(c1OCCCC)Cc1cc(CCCOC(=O)C(=C)CO[Si](C)(C)C(C)(C)C)cc(c1OCCCC)Cc1cc(CCCOC(=O)C(=C)CO[Si](C)(C)C(C)(C)C)cc(c1OCCCC)C2]

### Synthesis Example 77

The same procedure as in Synthesis Example 74 was repeated except that the compound obtained in Synthesis Example 62 (2.50 g, 2.38 mmol) was used instead of the compound obtained in Synthesis Example 59, to thereby obtain a compound represented by the following formula (3.305 g, yield 75.3%).

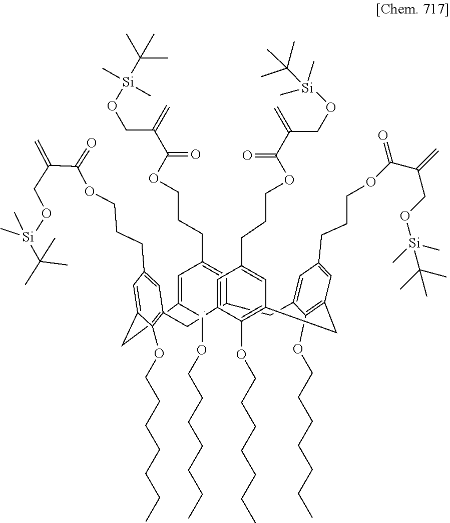 [SMILES: C=C(CO[Si](C)(C)C(C)(C)C)C(=O)OCCCc1cc2c(OCCCCCCC)c(c1)Cc1cc(CCCOC(=O)C(=C)CO[Si](C)(C)C(C)(C)C)cc(c1OCCCCCCC)Cc1cc(CCCOC(=O)C(=C)CO[Si](C)(C)C(C)(C)C)cc(c1OCCCCCCC)Cc1cc(CCCOC(=O)C(=C)CO[Si](C)(C)C(C)(C)C)cc(c1OCCCCCCC)C2]

### Synthesis Example 78

The same procedure as in Synthesis Example 74 was repeated except that the compound obtained in Synthesis Example 63 (2.50 g, 1.50 mmol) was used instead of the compound obtained in Synthesis Example 59, to thereby obtain a compound represented by the following formula (3.011 g, yield 81.6%).

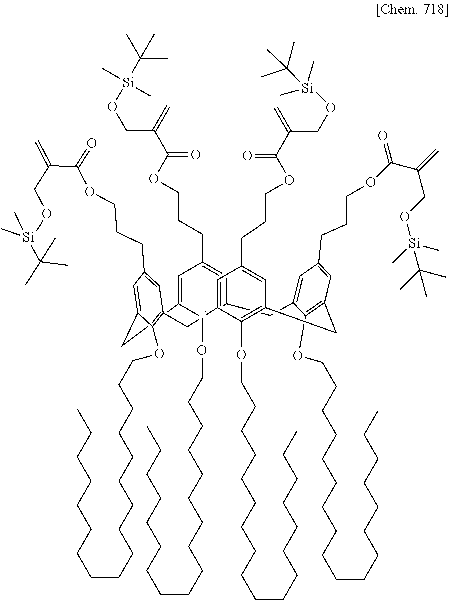 [SMILES: C=C(CO[Si](C)(C)C(C)(C)C)C(=O)OCCCc1cc2c(OCCCCCCCCCCCCCCCCCC)c(c1)Cc1cc(CCCOC(=O)C(=C)CO[Si](C)(C)C(C)(C)C)cc(c1OCCCCCCCCCCCCCCCCCC)Cc1cc(CCCOC(=O)C(=C)CO[Si](C)(C)C(C)(C)C)cc(c1OCCCCCCCCCCCCCCCCCC)Cc1cc(CCCOC(=O)C(=C)CO[Si](C)(C)C(C)(C)C)cc(c1OCCCCCCCCCCCCCCCCCC)C2]

### Synthesis Example 79

A 200 mL four-necked flask equipped with a stirrer, a thermometer, and a reflux condenser tube was charged with 3.50 g (1.96 mmol) of the compound obtained in Synthesis Example 74, 0.706 g (11.75 mmol) of acetic acid, and 78.4 mL of tetrahydrofuran, and the mixture was stirred. A colorless transparent solution. Then tetrabutylammonium fluoride (an about 1 mol/L tetrahydrofuran solution 11.75 mL (11.75 mmol) was slowly added dropwise to the mixture placed in an ice bath under stirring. The resulting mixture was stirred at room temperature for 12 hours. A saturated aqueous ammonium chloride solution was added to the reaction mixture, and then 50 mL of chloroform was added. The reaction mixture was transferred to a separatory funnel, and the organic layer was separated. Then the aqueous layer was extracted with 50 mL of chloroform twice. The combined organic layer was washed with saturated brine and dried over anhydrous magnesium sulfate. The solvent was removed by evaporation using an evaporator to thereby obtain a yellow transparent liquid. The yellow transparent liquid was purified by silica gel column column chromatography to thereby obtain a compound represented by the following formula (yield amount 2.417 g, yield 92.8%).

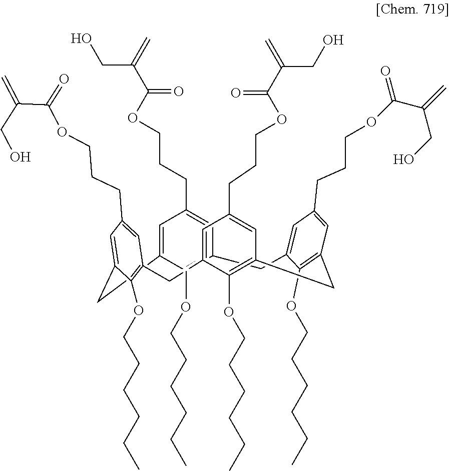 [SMILES: C=C(CO)C(=O)OCCCc1cc2c(OCCCCCC)c(c1)Cc1cc(CCCOC(=O)C(=C)CO)cc(c1OCCCCCC)Cc1cc(CCCOC(=O)C(=C)CO)cc(c1OCCCCCC)Cc1cc(CCCOC(=O)C(=C)CO)cc(c1OCCCCCC)C2]

### Synthesis Example 80

The same procedure as in Synthesis Example 79 was repeated except that the compound obtained in Synthesis Example 75 (3.50 g, 2.32 mmol) was used instead of the compound obtained in Synthesis Example 74, to thereby obtain a compound represented by the following formula (2.214 g, yield 90.8%).

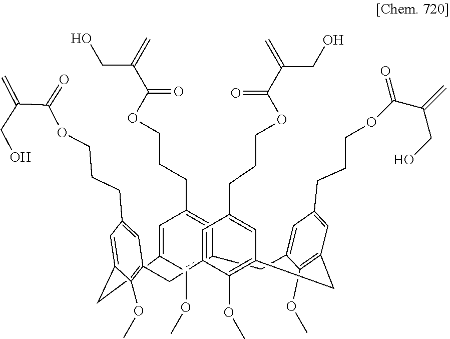 [SMILES: C=C(CO)C(=O)OCCCc1cc2c(OC)c(c1)Cc1cc(CCCOC(=O)C(=C)CO)cc(c1OC)Cc1cc(CCCOC(=O)C(=C)CO)cc(c1OC)Cc1cc(CCCOC(=O)C(=C)CO)cc(c1OC)C2]

### Synthesis Example 81

The same procedure as in Synthesis Example 79 was repeated except that the compound obtained in Synthesis Example 76 (3.50 g, 2.32 mmol) was used instead of the compound obtained in Synthesis Example 74, to thereby obtain a compound represented by the following formula (2.344 g, yield 92.1%).

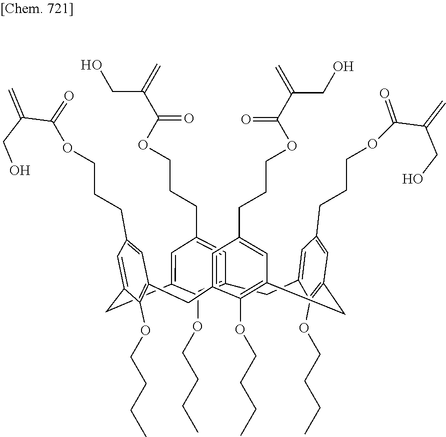 [SMILES: C=C(CO)C(=O)OCCCc1cc2c(OCCCC)c(c1)Cc1cc(CCCOC(=O)C(=C)CO)cc(c1OCCCC)Cc1cc(CCCOC(=O)C(=C)CO)cc(c1OCCCC)Cc1cc(CCCOC(=O)C(=C)CO)cc(c1OCCCC)C2]

### Synthesis Example 82

The same procedure as in Synthesis Example 79 was repeated except that the compound obtained in Synthesis Example 77 (3.50 g, 2.32 mmol) was used instead of the compound obtained in Synthesis Example 74, to thereby obtain a compound represented by the following formula (2.466 g, yield 93.7%).

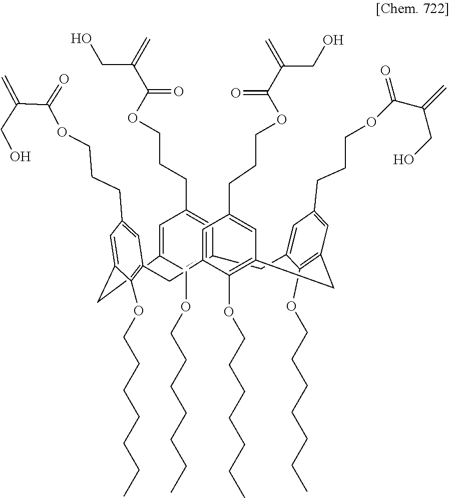 [SMILES: C=C(CO)C(=O)OCCCc1cc2c(OCCCCCCC)c(c1)Cc1cc(CCCOC(=O)C(=C)CO)cc(c1OCCCCCCC)Cc1cc(CCCOC(=O)C(=C)CO)cc(c1OCCCCCCC)Cc1cc(CCCOC(=O)C(=C)CO)cc(c1OCCCCCCC)C2]

### Synthesis Example 83

The same procedure as in Synthesis Example 79 was repeated except that the compound obtained in Synthesis Example 78 (3.50 g, 1.42 mmol) was used instead of the compound obtained in Synthesis Example 74, to thereby obtain a compound represented by the following formula (2.608 g, yield 91.5%).

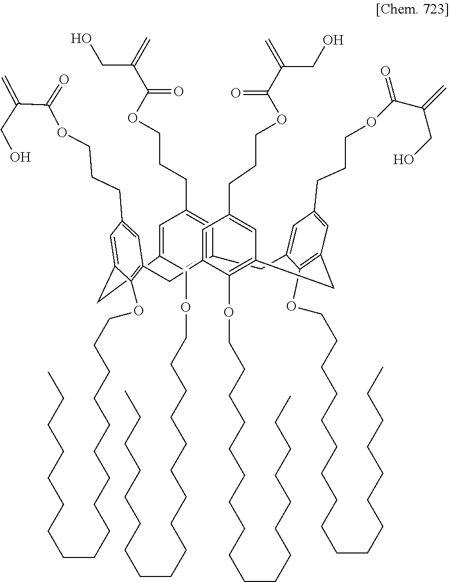 [SMILES: C=C(CO)C(=O)OCCCc1cc2c(OCCCCCCCCCCCCCCCCCC)c(c1)Cc1cc(CCCOC(=O)C(=C)CO)cc(c1OCCCCCCCCCCCCCCCCCC)Cc1cc(CCCOC(=O)C(=C)CO)cc(c1OCCCCCCCCCCCCCCCCCC)Cc1cc(CCCOC(=O)C(=C)CO)cc(c1OCCCCCCCCCCCCCCCCCC)C2]

### Example 48

A 100 mL four-necked flask equipped with a stirrer, a dropping funnel, and a thermometer was charged, in a nitrogen atmosphere, with the compound obtained in Synthesis Example 79 (2.0 g, 1.50 mmol), triethylamine (1.218 g, 12.0 mmol), and methylene chloride (19 mL), and the mixture was stirred under cooling with ice. A solution prepared by dissolving methyl malonyl chloride (1.232 g, 9.02 mmol) in methylene chloride (3 mL) was slowly added dropwise. After completion of the dropwise addition, the resulting mixture was stirred at room temperature for 8 hours. Water was added to the reaction mixture, and the resulting mixture was extracted with chloroform (40 mL) twice. The chloroform solution was washed with dilute hydrochloric acid, a saturated aqueous sodium hydrogencarbonate solution, and saturated brine and dried over anhydrous magnesium sulfate. The solvent was removed using an evaporator, and the obtained yellow viscous liquid was purified by silica gel column chromatography to thereby obtain the target compound 010-6 (yield amount 2.214 g, yield 85.1%).

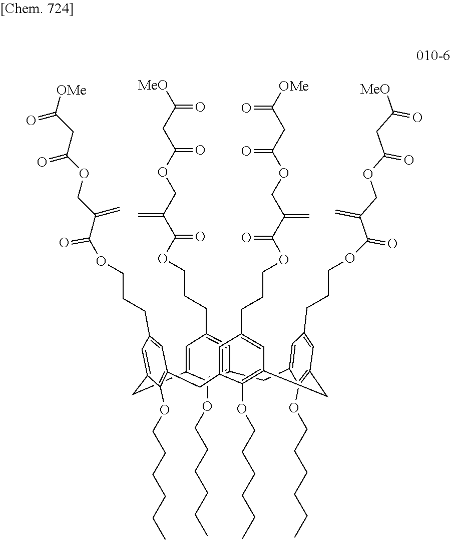 [SMILES: C=C(COC(=O)CC(=O)OC)C(=O)OCCCc1cc2c(OCCCCCC)c(c1)Cc1cc(CCCOC(=O)C(=C)COC(=O)CC(=O)OC)cc(c1OCCCCCC)Cc1cc(CCCOC(=O)C(=C)COC(=O)CC(=O)OC)cc(c1OCCCCCC)Cc1cc(CCCOC(=O)C(=C)COC(=O)CC(=O)OC)cc(c1OCCCCCC)C2]

### Example 49

The same procedure as in Example 48 was repeated except that the compound obtained in Synthesis Example 80 (2.00 g, 1.91 mmol) was used instead of the compound obtained in Synthesis Example 79, to thereby obtain the target compound 010-1 (2.304 g, yield 83.4%).

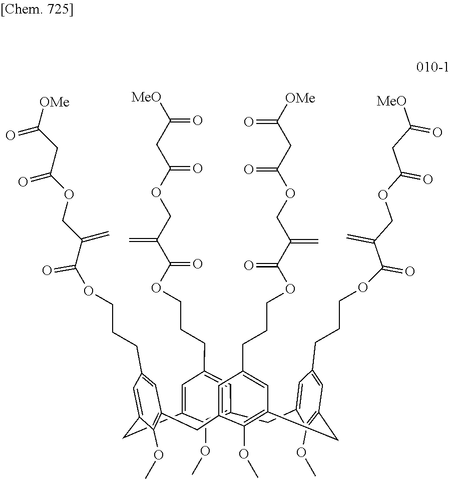 [SMILES: C=C(COC(=O)CC(=O)OC)C(=O)OCCCc1cc2c(OC)c(c1)Cc1cc(CCCOC(=O)C(=C)COC(=O)CC(=O)OC)cc(c1OC)Cc1cc(CCCOC(=O)C(=C)COC(=O)CC(=O)OC)cc(c1OC)Cc1cc(CCCOC(=O)C(=C)COC(=O)CC(=O)OC)cc(c1OC)C2]

### Example 50

The same procedure as in Example 48 was repeated except that the compound obtained in Synthesis Example 81 (2.00 g, 1.64 mmol) was used instead of the compound obtained in Synthesis Example 79, to thereby obtain the target compound 010-4 (2.299 g, yield 86.5%).

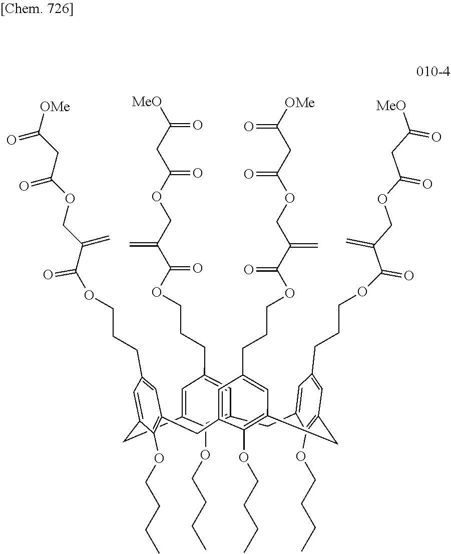 [SMILES: C=C(COC(=O)CC(=O)OC)C(=O)OCCCc1cc2c(OCCCC)c(c1)Cc1cc(CCCOC(=O)C(=C)COC(=O)CC(=O)OC)cc(c1OCCCC)Cc1cc(CCCOC(=O)C(=C)COC(=O)CC(=O)OC)cc(c1OCCCC)Cc1cc(CCCOC(=O)C(=C)COC(=O)CC(=O)OC)cc(c1OCCCC)C2]

### Example 51

The same procedure as in Example 48 was repeated except that the compound obtained in Synthesis Example 82 (2.00 g, 1.44 mmol) was used instead of the compound obtained in Synthesis Example 79, to thereby obtain the target compound 010-7 (2.286 g, yield 88.7%).

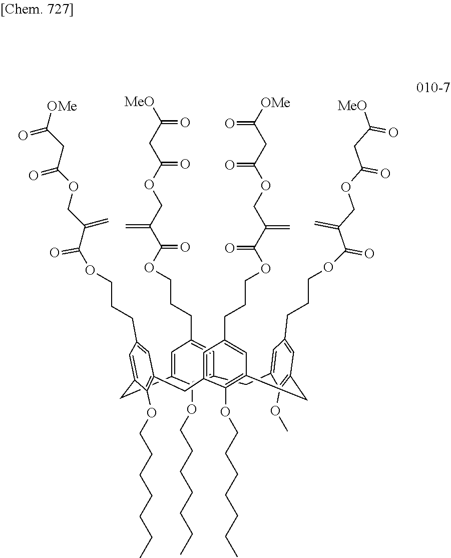 [SMILES: C=C(COC(=O)CC(=O)OC)C(=O)OCCCc1cc2c(OC)c(c1)Cc1cc(CCCOC(=O)C(=C)COC(=O)CC(=O)OC)cc(c1OCCCCCCC)Cc1cc(CCCOC(=O)C(=C)COC(=O)CC(=O)OC)cc(c1OCCCCCCC)Cc1cc(CCCOC(=O)C(=C)COC(=O)CC(=O)OC)cc(c1OCCCCCCC)C2]

### Example 52

The same procedure as in Example 48 was repeated except that the compound obtained in Synthesis Example 83 (2.00 g, 1.00 mmol) was used instead of the compound obtained in Synthesis Example 79, to thereby obtain the target compound 010-18 (1.956 g, yield 81.5%).

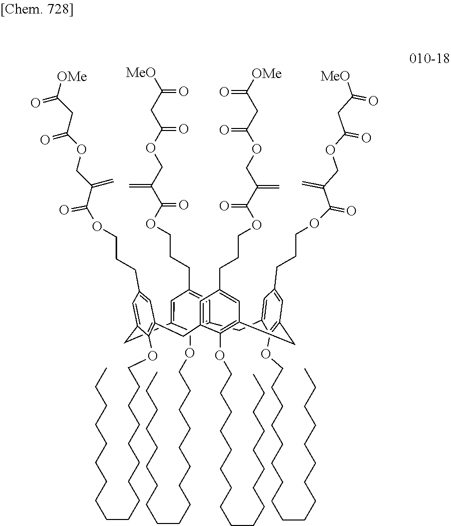 [SMILES: C=C(COC(=O)CC(=O)OC)C(=O)OCCCc1cc2c(OCCCCCCCCCCCCCCCCCC)c(c1)Cc1cc(CCCOC(=O)C(=C)COC(=O)CC(=O)OC)cc(c1OCCCCCCCCCCCCCCCCCC)Cc1cc(CCCOC(=O)C(=C)COC(=O)CC(=O)OC)cc(c1OCCCCCCCCCCCCCCCCCC)Cc1cc(CCCOC(=O)C(=C)COC(=O)CC(=O)OC)cc(c1OCCCCCCCCCCCCCCCCCC)C2]

### COMPARATIVE EXAMPLE

A 100 mL four-necked flask equipped with a stirrer, a thermometer, and a reflux condenser tube was charged with 1.00 g (1.212 mmol) of the compound obtained in Synthesis Example 20, 10.00 g (138.7 mmol) of tetrahydrofuran, 1.907 g (7.271 mmol) of triphenylphosphine, and 0.6260 g (7.271 mmol) of methacrylic acid, and the mixture was stirred. A light yellow transparent solution. Then 1.470 g (7.271 mmol) of diisopropyl azodicarboxylate was added dropwise to the mixture placed in an ice bath over 30 minutes. A light yellow transparent solution. The solution was stirred at room temperature for 6 hours. Hexane was added to the reaction solution to precipitate and remove by-products such as triphenylphosphine, and the resulting solution was extracted with chloroform, washed with water and saturated brine, and dried over magnesium sulfate. The solvent was removed by evaporation using an evaporator, and the resulting orange viscus liquid was subjected to column chromatography (eluent: n-hexane:acetone=90:10) to thereby obtain compound (1′) represented by the following formula. Compound (1′) was vacuum dried (at 60° C. for 6 hours or longer). 0.9058 g, and the yield was 68.1%.

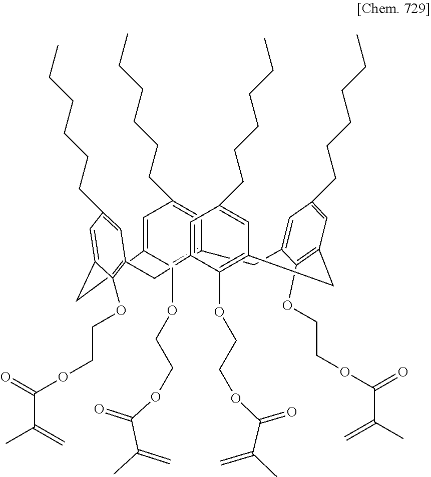 [SMILES: C=C(C)C(=O)OCCOc1c2cc(CCCCCC)cc1Cc1cc(CCCCCC)cc(c1OCCOC(=O)C(=C)C)Cc1cc(CCCCCC)cc(c1OCCOC(=O)C(=C)C)Cc1cc(CCCCCC)cc(c1OCCOC(=O)C(=C)C)C2]

<Production of Curable Compositions>

0.25 g of one of the obtained calixarene compounds, 0.25 g of dipentaerythritol hexaacrylate (“A-DPH” manufactured by Shin Nakamura Chemical Co., Ltd.), 0.005 g of a polymerization initiator (“Irgacure 369” manufactured by BASF), and 9.5 g of propylene glycol monomethyl ether acetate were mixed to obtain a curable composition.

<Production of Layered Bodies>

The curable composition was applied to substrates 1 to 4 below by a spin coating method such that the thickness of the coating after curing was about 0.5 μm and then dried on a hot plate at 100° C. for 2 minutes. A high-pressure mercury lamp was used to irradiate the curable composition with UV rays at 500 mJ/cm² in a nitrogen atmosphere to cure the curable composition, and layered bodies were thereby obtained.

Substrate 1: polymethyl methacrylate resin plate

Substrate 2: aluminum plate

Substrate 3: polyethylene terephthalate film having a SiO₂ thin layer (thickness 100 nm) (the curable composition was applied to the SiO: thin film)

<Evaluation of Adhesion>

A layered body stored in a 23° C. and 50% RH environment for 24 hours was used, and the adhesion was evaluated according to JIS K6500-5-6 (adhesive strength: a cross-cut method). A cellophane tape used was “CT-24” manufactured by Nichiban Co., Ltd. The criteria for the evaluation are as follows.

A: 80 or more out of 100 squares were not detached and remained present.

B: 50 to 79 out of 100 squares were not detached and remained present.

C: The number of squares that were not detached and remained present was 49 or less out of 100 squares.

<Evaluation of Moist Heat Resistance>

One of the curable compositions was applied to a 5 inch SiO substrate to a film thickness of about 50 μm using an applicator and dried on a hot plate at 100° C. for 2 minutes. A mask having an LS pattern with L/S=50 μm/50 μm was brought into tight contact with the coating obtained, and a high-pressure mercury lamp was used to irradiate the composition with UV rays at 1000 mJ/cm² in a nitrogen atmosphere to cure the composition. The substrate exposed to the light was developed using ethyl acetate to obtain an evaluation substrate. The obtained substrate was stored in a thermo-hygrostat at 85° C. and 85% RH for 100 hours, and a laser microscope (“VK-X200” manufactured by KEYENCE CORPORATION) was used to check the state of the pattern after a lapse of 100 hours. The criteria for the evaluation are as follows.

A: The entire pattern was well modified and maintained.

B: Cracking or chipping was observed in part of the pattern.

C: Cracking or chipping was observed in the pattern, and delamination of the pattern was also observed.

TABLE 32

| Calixarene compound | | 1-6 | 2-6 | 3-6 | 4-6 | 1-4 | 2-4 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 3-4 | 4-4 | 1-7 | 2-7 | 3-7 | 4-7 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 5-6 | 6-6 | 7-6 | 8-6 | 9-6 | 10-6 |
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 33

| Calixarene compound | | 11-6 | 12-6 | 9-4 | 10-4 | 11-4 | 12-4 |
|---|---|---|---|---|---|---|---|
| Adhesion | Substrate 1 | A | B | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 9-7 | 10-7 | 11-7 | 12-7 | 13-6 | 14-6 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | B |

| Calixarene compound | | 15-6 | 16-6 | 17-6 | 16-6 | 19-6 | 20-6 |
|---|---|---|---|---|---|---|---|
| Adhesion | Substrate 1 | A | A | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 34

| Calixarene compound | | 21-6 | 22-6 | 23-6 | 24-6 | 25-6 | 26-6 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 27-6 | 28-6 | 29-6 | 30-6 | 31-6 | 32-6 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 33-6 | 34-6 | 35-6 | 36-6 | 33-4 | 34-4 |
| Adhesion | Substrate 1 | A | B | A | B | A | B |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 35

| Calixarene compound | | 35-4 | 36-4 | 33-7 | 34-7 | 35-7 | 36-7 |
|---|---|---|---|---|---|---|---|
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 33-18 | 34-18 | 35-18 | 36-18 | 33-1 | 34-1 |
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 35-1 | 36-1 | 37-6 | 38-6 | 39-6 | 40-6 |
| Adhesion | Substrate 1 | A | B | A | A | A | B |
| | Substrate 2 | A | A | A | A | A | A |
| | Substrate 3 | A | A | A | A | A | A |

| | | | | | | |
|---|---|---|---|---|---|---|
| Moist heat resistance | A | B | A | A | A | A |

TABLE 36

| Calixarene compound | | 41-6 | 42-6 | 43-6 | 44-6 | 45-6 | 46-6 |
|---|---|---|---|---|---|---|---|
| Adhesion | Substrate 1 | A | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 47-6 | 48-6 | 49-6 | 50-6 | 51-6 | 52-6 |
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 53-6 | 54-6 | 55-6 | 56-6 | 57-6 | 58-6 |
| Adhesion | Substrate 1 | A | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 37

| Calixarene compound | | 59-6 | 60-6 | 61-6 | 62-6 | 63-6 | 64-6 |
|---|---|---|---|---|---|---|---|
| Adhesion | Substrate 1 | A | B | A | A | A | B |
| | Substrate 2 | A | A | A | A | A | A |
| | Substrate 3 | A | A | A | A | A | A |
| Moist heat resistance | | A | A | A | A | A | A |
| Calixarene compound | | 65-6 | 65-4 | 65-7 | 65-18 | 65-1 | 66-6 |
| Adhesion | Substrate 1 | A | A | A | A | A | A |
| | Substrate 2 | A | A | A | B | A | A |
| | Substrate 3 | A | A | A | A | A | A |

| | | | | | | |
|---|---|---|---|---|---|---|
| Moist heat resistance | A | A | A | A | A | A |

| Calixarene compound | | 67-6 | 68-6 | 01-6 | 02&03-6 | 04-6 | 01-1 |
|---|---|---|---|---|---|---|---|
| Adhesion | Substrate 1 | A | A | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 38

| Calixarene compound | | 02&03-1 | 04-1 | 01-4 | 02&03-4 | 04-4 | 01-7 |
|---|---|---|---|---|---|---|---|
| Adhesion | Substrate 1 | A | B | A | A | B | A |
| | Substrate 2 | A | A | A | A | A | A |
| | Substrate 3 | A | A | A | A | A | A |
| Moist heat resistance | | A | A | A | A | A | A |
| Calixarene compound | | 02&03-7 | 04-7 | 01-18 | 02&03-18 | 04-18 | 05-6 |
| Adhesion | Substrate 1 | A | B | A | A | B | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 05-1 | 05-4 | 05-7 | 05-18 | 06-6 | 07&08-6 |
|---|---|---|---|---|---|---|---|
| Adhesion | Substrate 1 | A | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 39

| Calixarene compound | | 09-6 | 06-1 | 07&08-1 | 09-1 | 06-4 | 07&08-4 |
|---|---|---|---|---|---|---|---|
| Adhesion | Substrate 1 | B | A | A | B | A | A |
| | Substrate 2 | A | A | A | A | A | A |
| | Substrate 3 | A | A | A | A | A | A |
| Moist heat resistance | | A | A | A | A | A | A |
| Calixarene compound | | 09-4 | 06-7 | 07&08-7 | 09-7 | 06-18 | 07&08-18 |
| Adhesion | Substrate 1 | B | A | A | B | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

| Calixarene compound | | 09-18 | 010-6 | 010-1 | 010-4 | 010-7 | 010-18 |
|---|---|---|---|---|---|---|---|
| Adhesion | Substrate 1 | B | A | A | A | A | A |
| | Substrate 2 | A | A | A | A | A | A |
| | Substrate 3 | A | A | A | A | A | A |

| | | | | | | |
|---|---|---|---|---|---|---|
| Moist heat resistance | A | A | A | A | A | A |

TABLE 40

| Calixarene compound | | 1′ |
|---|---|---|
| Adhesion | Substrate 1 | C |
| | Substrate 2 | C |
| | Substrate 3 | C |

| | |
|---|---|
| Moist heat resistance | B |

### INDUSTRIAL APPLICABILITY

The present invention can provide a calixarene compound having a novel structure that allows good solubility in a general purpose solvent and can provide a cured product excellent not only in properties such as heat resistance and hardness but also in properties such as adhesion to a substrate. The present invention can also provide a curable composition containing the calixarene compound and a cured product thereof. The calixarene compound of the present invention can be preferably used for various applications such as paints, printing inks, adhesives, resist materials, and interlayer dielectrics.

1. A calixarene compound represented by structural formula (1) below: 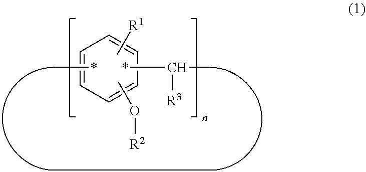
[wherein
R¹ and R² each independently represent a structural moiety (A) having a functional group (I) selected from the group consisting of a cyano group, maleate groups, an acetylacetonate group, oxalate groups, and malonate groups, a structural moiety (B) having a functional group (II) having a carbon-carbon unsaturated bond (excluding maleate groups), a structural moiety (C) having both the functional group (I) and the functional group (II), a monovalent organic group (D) that has 1 to 20 carbon atoms and is other than the structural moieties (A), (B) and (C), or a hydrogen atom (E);
R³ represents a hydrogen atom, an aliphatic hydrocarbon group optionally having a substituent, or an aryl group optionally having a substituent;
n is an integer of 2 to 10;
*s indicate the points of attachment to the aromatic ring;
a plurality of R¹s may be the same or different: a plurality of R²s may be the same or different; and a plurality of R³s may be the same or different,
provided that at least one of the plurality of R²s is the structural moiety (A), the structural moiety (B), the structural moiety (C), or the organic group (D);
when the functional group (I) is a cyano group, an acetylacetonate group, an oxalate group, or a malonate group, at least one of the plurality of R¹s and the plurality of R²s is the structural moiety (C), or at least one of the plurality of R¹s and the plurality of R²s is the structural moiety (A) and at least another one of the plurality of R¹s and the plurality of R²s is the structural moiety (B); and
when the functional group (I) is a maleate group, at least one of the plurality of R¹s and the plurality of R²s is the structural moiety (A) or the structural moiety (C)].

2. The calixarene compound according to claim 1, wherein the calixarene compound is represented by structural formula (1-1) below: 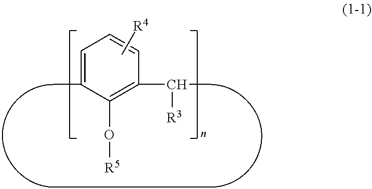
[wherein
R³ and n are the same as described above;
R⁴ represents a monovalent organic group (d1) having 1 to 20 carbon atoms and represented by —X—R (X is a direct bond or a carbonyl group, and R is a hydrogen atom or an aliphatic hydrocarbon group having 1 to 20 carbon atoms);
R⁵ represents the structural moiety (A), the structural moiety (B), the structural moiety (C), or a hydrogen atom (E);
a plurality of R³s may be the same or different; a plurality of R⁴s may be the same or different; and a plurality of R⁵s may be the same or different;
when the functional group (I) is a cyano group, an acetylacetonate group, an oxalate group, or a malonate group, at least one of the plurality of R⁵s is the structural moiety (C), or at least one of the plurality of R⁵s is the structural moiety (A) and at least another one of the plurality of R⁵s is the structural moiety (B);
when the functional group (I) is a maleate group, at least one of the plurality of R⁵s is the structural moiety (A) or the structural moiety (C)].

3. The calixarene compound according to claim 1, wherein the calixarene compound is represented by structural formula (1-2) below: 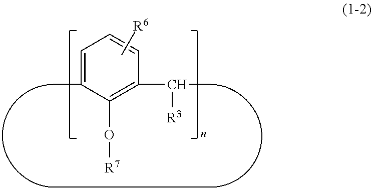
[wherein
R³ and n are the same as described above;
R⁶ represents the structural moiety (A), the structural moiety (B), or the structural moiety (C);
R⁷ represents an aliphatic hydrocarbon group (d2) having 1 to 20 carbon atoms;
a plurality of R³s may be the same or different; a plurality of R⁶s may be the same or different; a plurality of R⁷s may be the same or different;
when the functional group (I) is a cyano group, an acetylacetonate group, an oxalate group, or a malonate group, at least one of the plurality of R⁶s is the structural moiety (C), or at least one of the plurality of R⁶s is the structural moiety (A) and at least another one of the plurality of R⁶s is the structural moiety (B); and
when the functional group (I) is a maleate group, at least one of the plurality of R⁶s is the structural moiety (A) or the structural moiety (C)].

4. The calixarene compound according to claim 1, wherein the functional group (I) is a cyano group.

5. The calixarene compound according to claim 4, wherein the structural moiety (A) is a (poly)cyanoalkyl group or a group represented by structural formula (A-2) below: 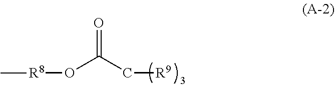
[wherein
R⁸ is an aliphatic hydrocarbon group or a direct bond, and
R⁹s are each independently a hydrogen atom, a hydroxy group, an alkyl group, or a (poly)cyanoalkyl group, at least one of R⁹s being a (poly)cyanoalkyl group].

6. The calixarene compound according to claim 4, wherein the structural moiety (C) is a group represented by structural formula (C-1) below, a group represented by structural formula (C-2) below, or a group represented by structural formula (C-3) below: 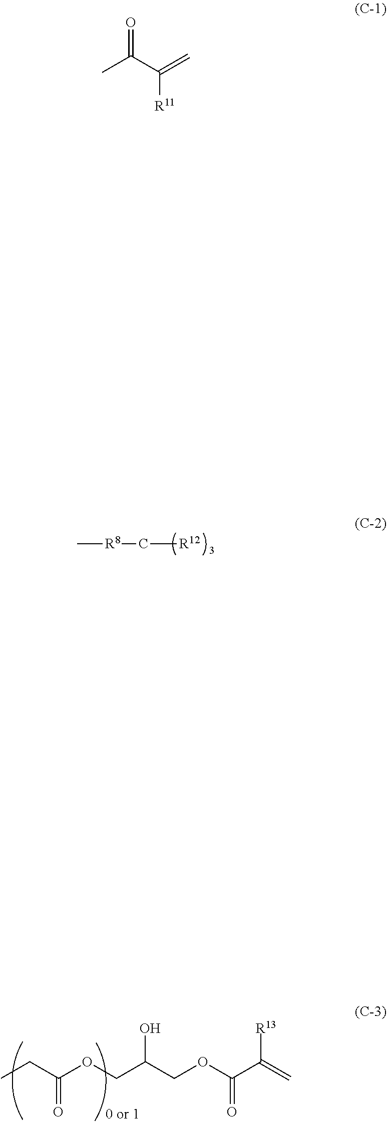
[wherein
R¹¹ is a (poly)cyanoalkyl group;
R⁸ is an aliphatic hydrocarbon group or a direct bond;
R¹²s are each independently a hydrogen atom, an alkyl group, a hydroxy group, a (poly)cyanoalkyl group, a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, a (meth)acryloylaminoalkyl group, or a group represented by structural formula (C-2-1) below: 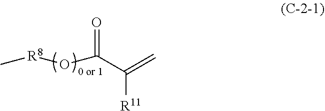
(wherein R⁸ and R¹¹ are the same as described above);
R¹³ is a (poly)cyanoalkyl group; and
at least one of the three R¹²s is a group represented by structural formula (C-2-1), or at least one of the three R¹²s is a (poly)cyanoalkyl group and at least another one of the three R¹²s is a vinyl group, a vinyloxy group, an allyl group, an allyloxy group, a propargyl group, a propargyloxy group, a (meth)acryloyl group, a (meth)acryloyloxy group, a (meth)acryloyloxyalkylene group, a (meth)acryloylamino group, or a (meth)acryloylaminoalkylene group].

7. The calixarene compound according to claim 1, wherein the functional group (I) is a maleate group.

8. The calixarene compound according to claim 7, wherein the structural moiety (A) is a group represented by structural formula (A-1) below: 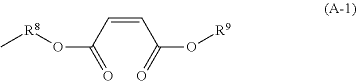
[wherein
R⁸ is an aliphatic hydrocarbon group or a direct bond, and
R⁹ is an aliphatic hydrocarbon group].

9. The calixarene compound according to claim 7, wherein the structural moiety (C) is a group represented by structural formula (C-1) below: 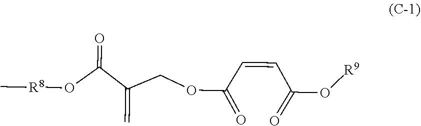
[wherein
R⁸ is an aliphatic hydrocarbon group or a direct bond, and
R⁹ is an aliphatic hydrocarbon group].

10. The calixarene compound according to claim 1, wherein the functional group (I) is an acetylacetonate group.

11. The calixarene compound according to claim 10, wherein the structural moiety (A) is a group represented by structural formula (A-1) below: 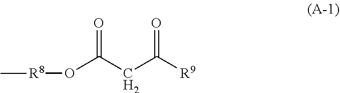
[wherein
R⁸ is an aliphatic hydrocarbon group or a direct bond, and
R⁹ is an aliphatic hydrocarbon group].

12. The calixarene compound according to claim 10, wherein the structural unit (C) is a group represented by structural formula (C-1) below: 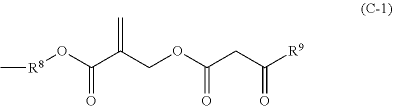
[wherein
R⁸ is an aliphatic hydrocarbon group or a direct bond, and
R⁹ is an aliphatic hydrocarbon group].

13. The calixarene compound according to claim 1, wherein the fictional group (I) is an oxalate group.

14. The calixarene compound according to claim 13, wherein the structural moiety (A) is a group represented by structural formula (A-1) below: 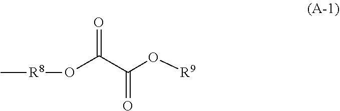
[wherein
R⁸ is an aliphatic hydrocarbon group or a direct bond, and
R⁹ is an aliphatic hydrocarbon group].

15. The calixarene compound according to claim 13, wherein the structural moiety (C) is a group represented by structural formula (C-1) below: 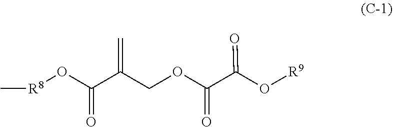
[wherein
R⁸ is an aliphatic hydrocarbon group or a direct bond, and
R⁹ is an aliphatic hydrocarbon group].

16. The calixarene compound according to claim 1, wherein the functional group (I) is a malonate group.

17. The calixarene compound according to claim 16, wherein the structural moiety (A) is a group represented by structural formula (A-1) below: 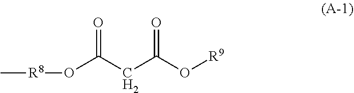
[wherein
R⁸ is an aliphatic hydrocarbon group or a direct bond, and
R⁹ is an aliphatic hydrocarbon group].

18. The calixarene compound according to claim 16, wherein the structural moiety (C) is a group represented by structural formula (C-1) below: 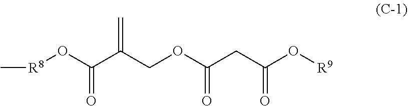
[wherein
R⁸ is an aliphatic hydrocarbon group or a direct bond, and
R⁹ is an aliphatic hydrocarbon group].

19. The calixarene compound according to claim 1, wherein the structural moiety (B) is a vinyl group, a propargyl group, a (meth)acryloyl group, a (meth)acryloylamino group, a group represented by structural formula (B-1) below, or a group represented by structural formula (B-2) below: 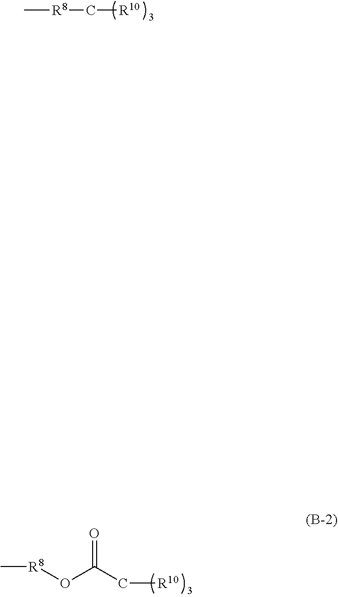
[wherein
R⁸s are each independently an aliphatic hydrocarbon group or a direct bond;
R¹⁰s are each independently a hydrogen atom, an alkyl group, a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group; and
at least one of the three R¹⁰s in each formula is a vinyl group, a vinyloxy group, a vinyloxyalkyl group, an allyl group, an allyloxy group, an allyloxyalkyl group, a propargyl group, a propargyloxy group, a propargyloxyalkyl group, a (meth)acryloyl group, a (meth)acryloyloxy group, a (meth)acryloyloxyalkyl group, a (meth)acryloylamino group, or a (meth)acryloylaminoalkyl group].

20. The calixarene compound according to claim 1, wherein n is 4.

21. A curable composition comprising the calixarene compound according to claim 1.

22. A cured product of the curable composition according to claim 21.
